(19) United States
(12) Patent Application Publication
(10) Pub. No.: US 2025/0276093 A1
(43) Pub. Date: Sep. 4, 2025

(54) THERAPEUTIC ADENO-ASSOCIATED VIRUS USING CODON OPTIMIZED NUCLEIC ACID ENCODING FACTOR VIII

(71) Applicant: ASKBIO INC., Research Triangle Park, NC (US)

(72) Inventors: Xavier ANGUELA MARTINEZ, Research Triangle Park, NC (US); Flavia SCIALPI, Research Triangle Park, NC (US); Sinclair COOPER, Research Triangle Park, NC (US); Juan Manuel IGLESIAS GONZALEZ, Research Triangle Park, NC (US); Lester SUAREZ, Research Triangle Park, NC (US)

(73) Assignee: ASKBIO INC., Research Triangle Park, NC (US)

(21) Appl. No.: 18/857,723

(22) PCT Filed: Apr. 20, 2023
(86) PCT No.: PCT/US2023/019211

§ 371 Date: Oct. 17, 2024

Related U.S. Application Data
(60) Provisional application No. 63/414,321, filed on Oct. 7, 2022.
(60) Provisional application No. 63/332,934, filed on Apr. 20, 2022.

(51) Int. Cl. A61K 48/00; A61K 9/00; A61K 38/00; A61P 7/04; C07K 14/755; C12N 15/86

(52) U.S. Cl. CPC A61K 48/0058; A61K 9/0019; A61K 48/0075; A61K 48/0083; A61P 7/04; C07K 14/755; C12N 15/86; A61K 38/00; C12N 2750/14143; C12N 2750/14152; C12N 2800/22; C12N 2830/50

(57) ABSTRACT

Disclosed herein are codon-optimized nucleic acids encoding a Factor VIII polypeptide. Also disclosed are expression cassettes and expression vectors (e.g., recombinant AAV (rAAV) vectors) that contain the codon-optimized nucleic acids in expressible form. Methods for the treatment of Hemophilia A comprising administering expression vector comprising the codon-optimized nucleic acids (e.g., a recombinant AAV (rAAV) vector) are also disclosed.

### CROSS-REFERENCE TO RELATED APPLICATIONS

This application claims benefit under 35 U.S.C. § 119(e) of U.S. Provisional Application No. 63/332,934 filed Apr. 20, 2022 and U.S. Provisional Application No. 63/414,321 filed Oct. 7, 2022, the contents of each of which are incorporated herein by reference in their entireties.

### SEQUENCE LISTING

The instant application contains a Sequence Listing as Table 3 herein, which is hereby incorporated by reference in its entirety.

### FIELD OF THE INVENTION

The present invention relates to methods to treat hemophilia A by administering adeno-associated virus (AAV) particles, virions and vectors for expression of a Factor VIII (FVIII) polypeptide, where the nucleic acid encoding FVIII is codon optimized.

### BACKGROUND

Hemophilia A and hemophilia B are X-linked bleeding disorders due to inheritable deficiencies in either coagulation factor VIII (FVIII) or factor IX (FIX), respectively (Peyvandi, et ak, Lancet (2016) 388:187-197; Konkle, et ah, Hemophilia A in GeneReviews, Adam, et ak, eds., University of Washington (1993)). The bleeding phenotype is generally related to the residual factor activity: people with severe disease (factor activity <1% normal) have frequent spontaneous bleeds; people with moderate disease (factor activity 1%-5% normal) rarely have spontaneous bleeds, but bleed with minor trauma; and people with mild disease (factor activity 5%-40% normal) bleed during invasive procedures or trauma.

To date, only 20% of patients with hemophilia A worldwide receive regular treatment with FVIII replacement therapy due its high cost. Typically, the FVIII is plasma-derived or recombinantly produced. Hemophilia A is a congenital X-linked bleeding disorder characterized by a deficiency in Factor VIII activity. Diminished Factor VIII activity inhibits a positive feedback loop in the coagulation cascade. This causes incomplete coagulation, which manifests as bleeding episodes with increased duration, extensive bruising, spontaneous oral and nasal bleeding, joint stiffness and chronic pain, and possibly internal bleeding and anemia in severe cases (Zhang et al., Clinic. Rev. Allerg. Immunol., 37:114-124 (2009)).

Conventionally, hemophilia A is treated by Factor VIII replacement therapy, which consists of administering Factor VIII protein (e.g., plasma-derived or recombinantly-produced Factor VIII) to an individual with hemophilia A. Factor VIII is administered prophylactically and/or perioperatively. However, there are several undesirable features of Factor VIII replacement therapy. Factor VIII replacement therapy does not cure the underlying Factor VIII deficiency, and continuous treatment is expensive and requires the individual to maintain strict compliance. Factor VIII has a relatively short half-life in vivo, requiring administration every second or third day. Between 15% and 30% of all individuals receiving Factor VIII replacement therapy form anti-Factor VIII inhibitor antibodies, rendering the therapy inefficient. FVIII typically loses its activity within miniutes after activatin by thrombin. WO2021/113800A1 describes a variant FVIII comprising mutatins at positions 336 and/or 562 wherein the Arg at these positions is substituted with Gln. This variant is expressed in a viral vector such as adeno-associated virus (AAV). However, there are safety limitations in using viral vectors because of mmune responses to the vector. Gene therapy to remedy the underlying condition of hemophilia A holds great promise but still faces challenges in implementaion. Improvement in expression and activity of delivered FVIII molecules will provide enhanced treastment options for hemophilia.

### SUMMARY OF THE INVENTION

The technology described herein relates generally to gene therapy constructs, methods and composition, for the treatment of Hemophilia A. More particularly, the technology relates to methods of using adeno-associated virus (AAV) particles configured for delivering a heterologous nucleic acid encoding FVIII polypeptide to a subject, and more particularly for delivering a heterologous codon optimized nucleic acid encoding FVIII polypeptide to a subject. These codon-optimized sequences reduce immunogenicity, while at the same time having high protency.

Aspects of the invention relate to a codon-optimized nucleic acid encoding a human Factor VIII (FVIII) polypeptide, wherein the encoded FVIII polypeptide lacks the B domain, and further comprises an amino acid substitution of Glutamine for Arginine at position 355 (R355Q) and of Glutamine for Arginine at position 581 (R581Q), wherein the nucleic acid comprises the nucleotide sequence set forth in SEQ ID NOs 1, 2, 4, 5, 7-9, 11-15 or 18, or a nucleic acid having at least 90%, 91%, 92%, 93%, 94%, 95%, 96%, 97%, or 98%, or 99% sequence identity thereto.

In some embodiments of the invention, nucleic acid comprises the nucleotide sequence set forth in SEQ ID NOs 4, 5, 7, 12-15 or 18, or a nucleic acid having at least 90%, 91%, 92%, 93%, 94%, 95%, 96%, 97%, or 98%, or 99% sequence identity thereto.

In some embodiments of the invention, the nucleic acid comprises the nucleotide sequence set forth in SEQ ID NOs 4, 5, 13, or 15, or a nucleic acid having at least 90%, 91%, 92%, 93%, 94%, 95%, 96%, 97%, or 98%, or 99% sequence identity thereto. In some embodiments of the invention, the nucleic acid comprises the nucleotide sequence set forth in SEQ ID NOs 4 or 5, or a nucleic acid having at least 90%, 91%, 92%, 93%, 94%, 95%, 96%, 97%, or 98%, or 99% sequence identity thereto.

In some embodiments of the invention, the human FVIII polypeptide is a functional variant of the human FVIII polypeptide having the amino acid sequence shown in SEQ ID NO: 19.

In some embodiments of the invention, the functional variant has least 60%, or 70%, or 80%, 85% or 90% or 95%, or 98%, or 99% sequence identity to the amino acid sequence shown in in SEQ ID NO: 19.

In some embodiments of the invention, the B domain of the encoded FVIII polypeptide has been replaced by a peptide linker.

In some embodiments of the invention, the encoded FVIII polypeptide lacks both the amino acid substitution of Glutamine for Arginine at position 355 (R355Q) and of Glutamine for Arginine at position 581 (R581Q).

In some embodiments of the invention, the encoded FVIII polypeptide lacks the amino acid substitution of Glutamine for Arginine at position 355 (R355Q).

In some embodiments of the invention, the encoded FVIII polypeptide lacks the amino acid substitution of Glutamine for Arginine at position 581 (R581Q).

In some embodiments of the invention, the codon-optimized nucleic acid is comprised within a nucleic acid construct that further comprises viral sequence elements that facilitate integration and expression.

Other aspects of the invention relate to an expression cassette containing the codon-optimized nucleic acid of any one of the above embodiments, operably linked to a constitutive promoter.

In some embodiments of the invention, the constitutive promoter is a TTR promoter.

In some embodiments of the invention, the TTR promoter comprises a nucleic acid sequence of SEQ ID NO: 431.

In some embodiments of the invention, the promoter is a liver-specific promoter.

In some embodiments of the invention, the liver specific promoter is selected from any of: SEQ ID NOS: 86, 88, 91-96, 146-150, 439-441, or 481-500, or a liver specific promoter having at least 80% sequence identity to SEQ ID NOs: 86, 88, 91-96, 146-150, 439-441, or 481-500.

In some embodiments of the invention, the liver specific promoter is selected from any of: SEQ ID NOS: 98 or 99, or a liver specific promoter having at least 80% sequence identity to SEQ ID NOs: 98 or 99.

In some embodiments of the invention, the liver specific promoter is SEQ ID NOS: 97, or a liver specific promoter having at least 80% sequence identity to SEQ ID NO: 97.

In some embodiments of the invention, the expression cassette further comprises one or more additional regulatory elements and/or a poly A sequence.

In some embodiments of the invention, the one or more additional regulatory elements is selected from the group consisting of an enhancer, a 5′ untranslated region (5′UTR), an intron, a reverse RNA pol II terminator sequence, and combinations thereof.

Other aspects of the invention relate to an expression cassette containing any of the codon-optimized nucleic acids descibred herein operably linked to a liver-specific promoter, wherein the liver specific promoter is selected from any of: SEQ ID NOS: 481-500, or a liver specific promoter having at least 80% sequence identity to SEQ ID NOs: 481-500.

Other aspects of the invention relate to an expression cassette containing any of the codon-optimized nucleic acids descibred herein operably linked to a liver-specific promoter, wherein the liver specific promoter is selected from any of: SEQ ID NOS: 481-483, or a liver specific promoter having at least 80% sequence identity to SEQ ID NOs: 481-483.

Other aspects of the invention relate to a recombinant adeno-associated virus (rAAV) vector comprising in its genome an expression cassettes described in any one of the above embodiments.

Other aspects of the invention relate to a recombinant adeno-associated virus (rAAV) vector comprising in its genome: 5′ and 3′ AAV inverted terminal repeats (ITR) sequences; and located between the 5′ and 3′ ITRs, the expression cassette specified in any one of the above embodiments.

In some embodiments of the invention, AAV genome further comprises at least one of: a 5′ ITR; an 5′ UTR sequence; an intron; a poly A sequence; a reverse RNA pol II terminator sequence; and a 3′ ITR.

In some embodiments of the invention, the AAV genome comprises, in the 5′ to 3′ direction: a 5′ ITR; a liver-specific promoter; a 5′ UTR sequence; an intron; a codon-optimized nucleic acid specified in any one of the above described embodiments; a poly A sequence; a reverse RNA pol II terminator sequence; and a 3′ ITR.

In some embodiments of the invention, the 5′ UTR sequence comprises SEQ ID NO: 41, or a nucleic acid having at least 90% sequence identity to SEQ ID NO: 41.

In some embodiments of the invention, the 5′ UTR sequence comprises SEQ ID NO: 40, or a nucleic acid having at least 90% sequence identity to SEQ ID NO: 40.

In some embodiments of the invention, the intron is selected from the group consisting of a MVM sequence, a HBB2 sequence, an CMVIE intron sequence, a UBC intron sequence, and a SV40 sequence.

In some embodiments of the invention, the 3′ UTR sequence is located 3′ of the codon-optimized nucleic acid and 5′ of the 3′ ITR sequence, or is located between the codon-optimized nucleic acid and the poly A sequence.

In some embodiments of the invention, the heterologous, codon-optimized nucleic acid sequence further comprises a 3′ intron sequence, wherein the 3′ intron sequence is located 3′ of the nucleic acid encoding the FVIII polypeptide and 5′ of the 3′ ITR sequence, or is located between the nucleic acid encoding the FVIII polypeptide and the poly A sequence.

In some embodiments of the invention, at least one of the 5′ ITR or 3′ITR comprises an insertion, deletion or substitution.

In some embodiments of the invention, one or more CpG islands in the ITR are removed.

In some embodiments of the invention, the poly A sequence is a full length HGF poly A sequence. In some embodiments, the poly A sequence is a 49 bp polyA as described in Levitt et al 1989 (doi: 10.1101/gad.3.7.1019), which is incorporated herein by reference in entirety (SEQ ID NO: 514). AATAAAAGATCTTTATTTTCATTAGATCTGTGTGTTGGTTTTTTGTGTG (SEQ ID NO: 514.

In some embodiments of the invention, poly A sequence is selected from SEQ ID NO: 42-44 or 514, or a nucleic acid sequence at least 80% sequence identity to SEQ ID NOS: 42-44 or 514.

In some embodiments of the invention, the reverse RNA pol II terminator sequence is SEQ ID NO: 45, or a nucleic acid sequence at least 80% sequence identity to SEQ ID NOS: 45.

In some embodiments of the invention, the rAAV vector is a chimeric AAV vector, haploid AAV vector, a hybrid AAV vector or polyploid AAV vector.

In some embodiments of the invention, the rAAV vector is a rational haploid vector, a mosaic AAV vector, a chemically modified AAV vector, or a AAV vector from any AAV serotypes.

In some embodiments of the invention, the rAAV vector is selected from the group consisting of: a AAVXL32 vector, a AAVXL32.1 vector, a AAV8 vector, or a haploid or, rational polyploid AAV8 vector comprising at least one AAV8 VP1, VP2, or, VP3 capsid protein.

In some embodiments of the invention, the rAAV vector has a capsid comprising capsid proteins from a serotype shown in Table 3 or a chimera thereof.

In some embodiments of the invention, the capsid proteins are serotype AAV3b.

In some embodiments of the invention, the AAV3b serotype capsid protein comprises one or more mutations selected from any of: 265D, 549A, Q263Y.

In some embodiments of the invention, the AAV3b serotype is selected from any of: AAV3b265D, AAV3b265D549A, AAV3b549A or AAV3bQ263Y, or AAV3bSASTG.

Other aspects of the invention relate to a pharmaceutical composition comprising the rAAV vector of any one of the above-described embodiments formulated in a pharmaceutically acceptable carrier.

Other aspects of the invention relate to a method for treating a subject in need of FVIII, the method comprising administering any one of the rAAV vectors, or the pharmaceutical compositions, or the expression cassettes, or the codon-optimized nucleic acids in any of the above-described embodiments. to the subject.

Other aspects of the invention relate methods for treating hemophilia A, the methods comprising administering any one of the rAAV vectors, or the pharmaceutical compositions, or the expression cassettes, or the codon-optimized nucleic acids in any of the above-described embodiments. to the subject.

In some embodiments of the methods described herein, the AAV vector is manufactured from the plasmid of SEQ ID NO: 27.

In some embodiments of the methods described herein, the encoded FVIII polypeptide is secreted from the subject's liver.

In some embodiments of the methods described herein, administering to the subject is by systemic administration.

In some embodiments of the methods described herein, the systemic administration is by intravenous administration.

In some embodiments of the methods described herein, administering to the subject is by local administration.

In some embodiments of the methods described herein, the local administration is by injection to the liver.

In some embodiments of the methods described herein, the rAAV vector is administered at a dosage range of between 1.0E9 vg/kg to 5.0E12 vg/kg.

Other aspects of the invention relate to the use of a rAAV vector in the preparation of a medicament for treating subject in need of FVIII, the medicament comprising the rAAV vector specified any one of the above-described embodiments.

Other aspects of the invention relate to the use of a rAAV vector in the preparation of a medicament for treating hemophilia A, the medicament comprising the rAAV vector specified any one of the above-described embodiments.

### BRIEF DESCRIPTION OF THE DRAWINGS

FIG. 1 is a graph of experimental results indicating in vitro levels of expression of human FVIII polypeptide in HepG2 and Huh7 cells from plasmids containing the 18 different nucleic acids generated in the codon optimization process, as compared to the benchmark nucleic acid. Shown are hFVIII activity levels in supernatant as measured by coatest. Values are shown relative to the average value resulting from transfection of the FVIII-QQ00 plasmid, a known codon-optimized FVIII based upon the SQ back-bone (which lacks the R to Q substitutions at positions 355 and 581) which is a B-domain delted FVIII. indicated by the dotted line at 1.0. In each group, different biological replicates, i.e., different transfection studies, are indicate by different symbols (circles vs triangles); the same symbol indicates technical replicates (N=3 per transfection).

FIG. 2 is a bar graph of experimental results indicating circulating hFVIII levels in mice that have been administered the plasmids containing the indicated FVIII nucleic acids, by hydrodynamic tail vein injection. Results are shown as hFVIII levels (% of normal) in mice that received a successful hydrodynamic injection. Values are represented as Mean±standard deviation. Values below the limit of quantification of 1.56% of normal hFVIII are shown as 1.56 for illustration purposes. The vertical dotted line separates experimental round 1 and 2 on the graph. * p<0.05 vs QQ00 Round 2, one-way ANOVA.

FIG. 3 is a bar graph of experimental results indicating in vivo levels of expression of human FVIII polypeptide in mice that have been administered rAAV particles containing the 18 different nucleic acids generated in the codon optimization process, as compared to the rAAV particles containin the benchmark nucleic acid. Shown are circulating hFVIII levels (% of normal) two- and four-weeks following administration of 1×10¹¹ vg/mouse of AAV8-FVIII-QQ. Values are represented as Mean±standard deviation. Values below the limit of quantification of 1.56% of normal hFVIII are shown as 1.56 for illustration purposes. * p<0.05 vs QQ00 at Day 28, one-way ANOVA. The solid line (red) indicates the expression level of the QQ00 benchmark, the dotted line indicates 2-fold increase over the QQ00 benchmark.

FIG. 4 is a bar graph showing circulating hFVIII levels as percent of normal (where 100% is equal to the amount in a healthy adult) at week 2 (d14) and week 4 (d28) following administration of 1×10¹⁰ vg/mouse of AAV8-FVIII expressed with the indicated promoter. HLP is a control promoter.

FIG. 5 is a bar graph showing the vector copies normalized (VCN) per diploid genome in liver at week 4 after the administration of 1×10¹⁰ vg/mouse of AAV8-FVIII expressed with the indicated promoter. Closed circles represent individual mice. Bars and lines represent mean and SD, respectively. HLP is a control promoter.

FIG. 6 is a bar graph showing circulating hFVIII levels normalized per VCN in liver at week 4 after the administration of 1×10¹⁰ vg/mouse of AAV8-FVIII expressed with the indicated promoter. HLP is a control promoter.

FIG. 7 presentes bar graphs showing relative expression levels of AAV8-FVIII expressed with the indicated promoter in the indicated organs at terminal sacrifice after administration. The graph shows the fold change relative to vector-derived FVIII expression in the liver in each animal. HLP is a control promoter. (N=5 mice per group)

FIG. 8 is a bar graph showing the expression of hFVIII at weeks 2 and 4 post administration of 1×10¹⁰ vg/mouse of AAV8-FVIII expressed with the indicated promoter. The first bar of each indicated promoter is 2 weeks post administration. The second bar of each indicated promoter is 4 weeks post administration. HLP is a control promoter.

FIG. 9 is a bar graph showing ELISA expression of hFVIII normalized for VCN at week 4 post administration of 1×10¹⁰ vg/mouse of AAV8-FVIII expressed with the indicated promoter. All promoters show stronger hFVIII expression than benchmark promoter (HLP).

FIG. 10 is a bar graph showing circulating hFVIII levels as percent of normal (where 100% is equal to the amount in a healthy adult) at week 2 (d14) and week 4 (d28) following administration of 1×10¹⁰ vg/mouse of AAV8-FVIII expressed with the indicated promoter. HLP is a control promoter.

FIG. 11 is a bar graph showing the vector copies normalized (VCN) per diploid genome in liver at week 4 after the administration of 1×10¹⁰ vg/mouse of AAV8-FVIII expressed with the indicated promoter. Closed circles represent individual mice. Bars and lines represent mean and SD, respectively. HLP is a control promoter.

FIG. 12 is a bar graph showing circulating hFVIII levels normalized per VCN in liver at week 4 after the administration of 1×10¹⁰ vg/mouse of AAV8-FVIII expressed with the indicated promoter. HLP is a control promoter.

FIG. 13A are bar graphs showing circulating hFVIII levels as percent of normal (where 100% is equal to the amount in a healthy adult) at 14 (top) and 28 (bottom) days following administration of 5e9 vg/mouse AAV8-FVIII expressed with the indicated promoter. TTR refers to a TTR-SQ reference construct used as a control. FVIII level is significantly more than the benchmark control FVIII expressed by TTR-SQ construct.

FIG. 13B are bar graphs showing the vector copy numbers (VCN) in liver at 28 days after the administration of 5e9 vg/mouse AAV8-FVIII expressed with the indicated promoter. TTR refers to a TTR-SQ reference construct used as a control. FVIII levels normalized over VCN is significantly more than the benchmark control FVIII expressed by TTR-SQ construct normalized over VCN.

FIG. 14A are bar graphs showing circulating hFVIII levels as percent of normal (where 100% is equal to the amount in a healthy adult) at 14 (top) and 28 (bottom) days following administration of 1.68e9 vg/mouse (a suboptimal dilution) AAV8-FVIII expressed with the indicated promoter. TTR refers to a TTR-SQ reference construct used as a control. FVIII level is significantly more than the benchmark control FVIII expressed by TTR-SQ construct.

FIG. 14B are bar graphs showing the vector copy numbers (VCN) in liver at 28 days after the administration of 1.68e9 vg/mouse (a suboptimal dilution) AAV8-FVIII expressed with the indicated promoter. TTR refers to a TTR-SQ reference construct used as a control. FVIII levels normalized over VCN is significantly more than the benchmark control FVIII expressed by TTR-SQ construct normalized over VCN.

### DETAILED DESCRIPTION

Aspects of the invention described herein arise from the identification of codon-optimized nucleic acids that encode a human Factor VIII (FVIII) polypeptide. These codon-optimized nucleic acids can be used to produce vectors for gene therapy (e.g., AAV based gene therapy) to treat disorders related to aberrant FVIII in a subject, e.g., Hemophilia A. Recombinant vectors (e.g., AAV) vectors and expression cassetts that contain the codon-optimized nucleic acid are used to deliver the FVIII coding sequence in expressible form, to the subject. The nucleic acid encoding the FVIII polypeptide described herein is codon optimized for enhanced expression in human subjects. That is, the rAAV vectors described herein for delivering a FVIII polypeptide to a subject comprise improvements, such as but not limited to, a codon optimized nucleic acid sequence encoding a FVIII polypeptide, where the codon optimized nucleic acid sequence encoding the FVIII polypeptide is modified to include features for example, to reduce CpG islands and/or minimize alternative open reading frames and/or maximize sequence diversity.

Furthermore, recombinant AAV (rAAV) vector and constructs described herein for delivering the FVIII polypeptide to a subject comprise improvements such as, e.g., incorporation of a 5′ UTR located between the nucleic acid expressing the FVIII polypeptide and the promoter, and use of specific terminator sequences 3′ nucleic acid expressing the FVIII polypeptide, such as, e.g., specific poly A sequences and/or terminator sequences.

In particular, described herein are viral vectors, e.g., using rAAV vectors as a non-limiting example, that comprise a nucleotide sequence containing inverted terminal repeats (ITRs), a promoter (e.g., a TTR promoter or liver specific promoter), a heterologous gene, a poly-A tail and potentially other regulator elements for use to treat a disease associated with aberrant FVIII expression (e.g., hemophelia A), where the heterologous gene is codon-optimized nucleic acid encoding a human FVIII polypeptide. In some embodients, the vector, e.g., rAAV, can be administered to a patient in a therapeutically effective dose that is delivered to the appropriate tissue and/or organ for expression of the heterologous gene and treatment of the disease, e.g., Hemophilia A.

One aspect of the invention relates to codon-optimized nucleic acids that encode a human FVIII polypeptide. In some embodiments, the encoded FVIII polypeptide lacks the B domain and further comprises an amino acid substitution of Glutamine for Arginine at position 355 (R355Q) and of Glutamine for Arginine at position 581 (AR581Q). In some embodiments, the nucleic acid has the nucleotide sequence set forth in SEQ ID NOs 1-18, or a nucleic acid having at least 90%, 91%, 92%, 93%, 94%, 95%, 96%, 97%, or 98%, or 99% sequence identity thereto. In some embodiments, the encoded FVIII polypeptide has the amino acid sequence set forth in SEQ ID NO: 19. In some embodiments, the encoded FVIII polypeptide is a functional variant of a polypeptide with the amino acid sequence set forth in SEQ ID NO: 19. In some embodiments, the encoded FVIII polypeptide lacks both the amino acid substitution of Glutamine for Arginine at position 355 (R355Q) and of Glutamine for Arginine at position 581 (R581Q). In some embodiments, the encoded FVIII polypeptide lacks one of the amino acid substitution of Glutamine for Arginine (either at position 355 (R355Q) or position 581 (R581Q)). In some embodiments, the nucleic acid has the nucleotide sequence set forth in one of SEQ ID NOs 1, 2, 4, 5, 7-9, 11-15 or 18, or a nucleic acid having at least 90%, 91%, 92%, 93%, 94%, 95%, 96%, 97%, or 98%, or 99% sequence identity thereto or any points in between. In some embodiments, the nucleic acid has the nucleotide sequence set forth in one of SEQ ID NOs 4, 5, 7, 12-15 or 18, or a nucleic acid having at least 90%, 91%, 92%, 93%, 94%, 95%, 96%, 97%, or 98%, or 99% sequence identity thereto. In some embodiments, the nucleic acid has the nucleotide sequence set forth in one of SEQ ID NOs 4, 5, 13, or 15, or a nucleic acid having at least 90%, 91%, 92%, 93%, 94%, 95%, 96%, 97%, or 98%, or 99% sequence identity thereto. In some embodiments, the nucleic acid has the nucleotide sequence set forth in one of SEQ ID NOs 4 or 5, or a nucleic acid having at least 90%, 91%, 92%, 93%, 94%, 95%, 96%, 97%, or 98%, or 99% sequence identity thereto.

In some embodiments, the codon-optimized nucleic acid described herein is operatively linked to a promoter to thereby generate an expression cassette. In some embodiments, the codon-optimized nucleic acid is included in an expression vector in expressible form (e.g., a viral based expression vector). Such expression vectors include nucleic acid constructs in the form of plasmids that comprise viral sequence elements (e.g., that facilitate integration and expression) and also recombinant viral particles.

### Recombinant AAV Expressing FVIII Polypeptide

As disclosed herein, one aspect of the technology relates to the use of the codon-optimized nucleic acid encoding the human FVIII polypeptide, described herein, in the treatment of disease (e.g, Hemophilia A). Aspects of the invention relate to an rAAV vector that contains and expresses the codon-optimized nucleic acid. The rAAV vector comprises a capsid, and within its capsid, a nucleotide sequence referred to as the “rAAV vector genome”. The rAAV vector genome (also referred to as “rAAV genome”) typically includes multiple elements required for expression of a heterologous gene contained therein, including, but not limited to two inverted terminal repeats (ITRs, e.g., the 5′-ITR and the 3′-ITR), and located between the ITRs are additional elements, including a promoter, the heterologous gene and a poly-A tail (e.g., SEQ ID NO: 21). The heterologous gene for use in the methods comprises the codon-optimized nucleic acid encoding a human FVIII polypeptide, described herein.

In one aspect, the invention relates to a rAAV vector comprising in its genome the codon-optimized nucleic acid encoding a human Factor VIII (FVIII) polypeptide, described herein. The encoded FVIII polypeptide lacks the B domain and further comprises an amino acid substitution of Glutamine for Arginine at position 355 (R355Q) and of Glutamine for Arginine at position 581 (R581Q). The nucleic acid has the nucleotide sequence set forth in one of SEQ ID NOs 1-18, or a nucleic acid having at least 60%, or 70%, or 80%, 85% or 90% or 95%, or 98%, or 99% sequence identity thereto.

In some embodiments, the rAAV vector has in its genome: (a) 5′ and 3′ AAV inverted terminal repeats (ITR) sequences, and (b) the codon-optimized nucleic acid encoding a human FVIII polypeptide that lacks the B domain and further has an amino acid substitution of Glutamine for Arginine at position 355 (R355Q) and of Glutamine for Arginine at position 581 (R581Q). The heterologous nucleic acid comprises the nucleotide sequence set forth in one of SEQ ID NOs 1-18, or a nucleic acid having at least 60%, or 70%, or 80%, 85% or 90% or 95%, or 98%, or 99% sequence identity thereto, and is located between the 5′ and 3′ ITRs in expressible form (e.g., the heterologous nucleic acid is operatively linked to a promoter as disclosed herein).

In some emboidments, the AAV genome further contains at least one of a 5′ ITR, a promoter sequence, a 5′ UTR sequence, a poly A sequence, a reverse RNA pol II terminator sequence and a 3′ ITR. In some embodidments, the AAV genome comprises, in the 5′ to 3′ direction, the 5′ ITR, the promoter sequence, e.g., a liver-specific promoter, the 5′ UTR sequence, the codon optimized nucleic acid, the poly A sequence, the reverse RNA pol II terminator sequence, and the 3′ ITR. In some embodiments of any aspect herein, the encoded human FVIII polypeptide further contains a peptide linker in the location of the omitted B domain. In some embodiments, the promoter is a liver specific promoter (e.g., has the sequence set forth in SEQ ID NOS: 97, 98 or 99, or a liver specific promoter having at least 80% sequence identity thereto). In some emboidments, the encoded human FVIII polypeptide has the amino acid sequence set forth in SEQ ID NO: 19, or is a functional variant thereof.

In certain embodiments, the liver specific promoter expresses the hFVIII polypeptide preferentially in the liver. In some embodiments, the AAV vector comprises at least one capsid protein that targets the liver.

In one embodiment of any aspect herein, the human FVIII polypeptide encoded by the codon-optimized nucleic acid has the amino acid sequence shown in SEQ ID NO: 19. In one embodiment, the human FVIII polypeptide is a functional variant of the human FVIII polypeptide having the sequence of SEQ ID NO: 19, as defined herein.

In some embodiments, the rAAV genome disclosed herein comprises a 5′ ITR and 3′ ITR sequence, and located between the 5′ITR and the 3′ ITR, a promoter, e.g., a TTR or liver-specific promoter, which is operatively linked to a heterologous nucleic acid encoding a Factor FVIII (FVIII) polypeptide, where the heterologous nucleic acid is codon optimized as disclosed herein, and where there is a 5′ untranslated region (5′ UTR) located between the nucleic acid encoding a FVIII polypeptide and the promoter sequence. In one embodiment, the heterologous nucleic acid sequence can optionally further comprise one or more of the following elements: an intron sequence, a poly A sequence, and a terminator sequence. In some embodiments, the 5′ UTR sequence comprises SEQ ID NO: 41, or comprises SEQ ID NO: 40, or a sequence having at least 85%, or at least 90% or more sequence identity to SEQ ID NOs: 40 or 41. In some embodiments, the poly A sequence is a full length HGH poly A sequence comprising SEQ ID NO: 42, or a sequence having at least 85%, or at least 90% or more sequence identity to SEQ ID NO: 42. In some embodiments, the terminator sequence is a reverse RNA pol II terminator sequence. In some embodiments, a reverse RNA pol II terminator sequence comprises sequence SEQ ID NO: 45, or a sequence having at least 85%, or at least 90% or more sequence identity to SEQ ID NO: 45.

### Coagulation Factor VIII (FVIII)

Factor VIII, as it is found in nature, is central for coagulation activity and mutations in the FVIII gene result in hemophilia A, the most common form of hemophilia. Full-length FVIII is a large, 280-kDa protein primarily expressed in liver sinusoidal endothelial cells (LSECs), as well as extra-hepatic endothelial cells (Fahs, et al., Blood (2014) 123:3706-3713; Everett, et al., Blood (2014) 123:3697-3705). Native FVIII predominantly circulates as a heterodimer of a heavy chain and a light chain bound through noncovalent metal-dependent interactions (Lenting, et al., Blood (1998) 92:3983-3996). Native Factor VIII comprises several domains and is 2332 amino acids in length (mature without signal peptide). Generally, the domains are referred to as A1-A2-B-A3-C1-C2. The FVIII gene is translated into a single-peptide chain with the domain structure of A1-a1-A2-a2-B-a3-A3-C1-C2. Proteolytic cleavage of FVIII at R-1313 and/or R-1648 by the trans-Golgi protease furin results in heterodimer formation. The FVIII heavy chain (A1-a1-A2-a2-B) and light chain (a3-A3-C1-C2) remain associated through non-covalent metal-ion-dependent interactions occurring between the A1 and A3 domains. Initially, FVIII is in an inactive form bound to von Willebrand factor (vWF). FVIII is activated by cleavage by thrombin (Factor IIa) and release of the B domain. The activated form of FVIII (FVIIIa) separates from vWF and interacts with coagulation factor Factor IXa-leading to the formation of a blood clot via a coagulation cascade. During coagulation, FVIII single chain or heterodimer is activated to its heterotrimeric cofactor form by cleavage by thrombin at R-372, R-740, and R-1689. A2 remains associated with A1-a1 via non-covalent interactions. Inactivation of FVIIIa occurs via spontaneous A2 dissociation and/or proteolytic cleavage, primarily by activated protein C, at R-336 and R-562.

Specific changes in the amino acid sequence of native FVIII are known to be associated with enhanced activity (e.g., via protein resistance to proteolytic inactivation). It has been found that the FVIII B domain is dispensable for procoagulant activity. Consequently, FVIII constructs in which the B domain is deleted are typically used for gene transfer purposes since their smaller size is more easily incorporated into vectors. Furthermore, it has been shown that deletion of the B domain leads to a 17-fold increase in mRNA and primary translation product. FVIII wherein the B domain is deleted and replaced (e.g., by a short amino acid linker, such as a 14 a.a. linker) are currently used clinically for protein replacement therapy.

Similarly, The FVIII polypetides encoded in gene therapy are typically engineered to be single chain polypeptides. Single-chain Factor VIII polypeptides have had the natural cleavage sites removed, and optionally have omitted, truncated B domains, or the B domains have been replaced with an alternative sequence. As such, they are not matured by cleavage (other than cleavage of a signal and/or leader peptide), and are active as a single chain. Non-limiting examples of single-chain Factor VIII polypeptides are described in Zollner et al. (Thromb Res, 134(1):125-31 (2014)) and Donath et al. (Biochem J., 312(1):49-55 (1995)), the contents of which are hereby incorporated by reference. Gene therapy using AAV vectors can only use shortened FVIII molecules such as a BDD-FVIII due to the limited packaging capacity of the AAV (4.7 Kb) and other vector systems (Lind, et al. (1995) Eur. J. Biochem., 232(1): 19-27).

The B domain comprises 40% of the native protein (908 amino acids) and is not required for the protein procoagulant activity (Brinkhous, et al., Proc. Natl. Acad. Sci. (1985) 82:8752-8756). Functional variants of the human FVIII polypeptide described herein include various iterations of B domain deletions, optionally including replacement with a linker. The most common B-domain deleted (BDD) FVIII comprises 14 original amino acid residues (SFSQNPPVLKRHQR (SEQ ID NO: 23) as a linker (Lind, et al. (1995) Eur. J. Biochem., 232(1): 19-27). This BDD FVIII is typically referred to as BDD-SQ or hFVIII-SQ. Short peptide linkers (e.g., 25 or fewer amino acids, 20 or fewer amino acids, 15 or fewer amino acids, or 10 or fewer amino acids) substituted for the B-domain can also be used in FVIII polypeptide variants (Lind, et al. (1995) Eur. J. Biochem., 232(1): 19-27; Pittman, et al., Blood (1993) 81:2925-2935; Toole, et al., Proc. Natl. Acad. Sci. (1986) 83:5939-5942). In some variants, the peptide linker comprises a basic amino acid (e.g., Arg, His, or Lys) at position −1 and −4 to Glu1649. This BDD FVIII form is commonly used to produce recombinant BDD-FVIII (˜4.4 Kb) as well for gene therapy (Bemtorp, E., Semin. Hematol. (2001) 38(2 Suppl 4): 1-3; Gouw, et al., N. Engl. J. Med. (2013) 368:231-239; Xi, et al., J. Thromb. Haemost. (2013) 11:1655-1662; Recht, et al., Haemophilia (2009) 15:869-880; Sabatino, et al., Mol. Ther. (2011) 19:442-449; Scallan, et al., Blood (2003) 102:2031-2037). U.S. Pat. No. 8,816,054, incorporated by reference herein, also provides BDD FVIII molecules with linkers of different lengths and sequences.

In some embodiments, the human FVIII polypeptide is deleted for the B domain (also referred to as B domain deleted FVIII, also referred to as BDD FVIII or FVIIIAB or FVIIIdeltaB herein). The term “B domain deleted FVIII” encompasses for example, but without limitation, FVIII polypeptides wherein whole or a part of the B domain is deleted and FVIII mutants wherein the B domain is replaced by a linker. Non-limiting examples of B domain deleted FVIII are described in Ward et al. (2011) and WO 2011/005968, which are specifically incorporated by reference herein.

In preferred embodiments, the FVIII polypeptide is B domain deleted and there is no further replacement of the domain.

Amino acid substitutions have been introduced at the two known FVIII APC cleavages sites, Arg355 and Arg581 (amino acid numbering refers to sequence that includes the signal peptide), generating a FVIII polypeptide that is resistant to APC cleavage. The specific amino amino acid substitutions of Q for R at these sites (FVIII-R355Q/R581Q [FVIII-QQ]) are reflected in the FVIII polypeptide sequence of SEQ ID NO: 19. Consistent with APC having a significant in vivo role in FVIIIa regulation, the FVIII-QQ demonstrates superior hemostatic efficacy relative to wild-type FVIII in an APC-dependent manner. Functional variants of the human FVIII polypeptide described herein include those resulting from amino acid substitutions of the SEQ ID NO: 19 amino acid sequence. It is expected that different amino acids can be substituted at the position 355 and/or 581 positions to generate functional variants of the human FVIII polypeptides described herein. This includes substitution of Arginine to revert back to the wild type sequence at one or more of these sites. In one embodiment, the FVIII comprises a substitution at position 355 that is not with Gln (Q). In one embodiment, the amino acid at position 355 is substituted with Lys (K), Asp (D), Glu (E), or Asn (N). In one embodiment, the amino acid at position 355 is substituted with Asn (N).

In one embodiment, the FVIII comprises a substitution at position 581 that is not with Gln (Q). In one embodiment, the amino acid at position 581 is substituted with Lys (K), Asp (D), Glu (E), or Asn (N). In one embodiment, the amino acid at position 581 is substituted with Asn (N).

It has further been shown that the FVIII polypeptide that has only one of the R355Q or the R581Q substitutions reflected in SEQ ID NO: 19 also exhibit superior hemostatic efficacy. As such, functional variants of the human FVIII with a single one of the R355Q or the R581Q substitutions, or substituted with Lys (K), Asp (D), Glu (E), or Asn (N), are further envisioned.

### Human Factor VIII Gene

The native human factor VIII (FVIII) gene has been characterized (Gene ID: 2157; Ensembl:ENSG00000185010 MIM:300841; AllianceGenome:HGNC:3546; UniProtKB—P00451). GenBank Accession Nos. NM_000132.3 and NP_000123.1 provide examples of the nucleotide and amino acid sequences of wild-type native human FVIII. The Factor VIII gene produces alternatively spliced transcripts. Transcript variant 1 encodes the large glycoprotein (sometimes referred to as isoform a), which is synthesized as a single chain polypeptide of 2351 amino acids. A 19-amino acid signal peptide is cleaved by a protease shortly after synthesis so that circulating plasma factor VIII is a heterodimer. This circulates in plasma and associates with von Willebrand factor in a noncovalent complex. This is considered the canonical isoform. One example of the protein sequence of isoform a is shown in Table 3 as SEQ ID NO: 24. This protein undergoes multiple cleavage events. The other transcript variants encode a smaller protein, one example of which is isoform b, which consists primarily of the phospholipid binding domain of factor VIIIc. This binding domain is essential for coagulant activity. One example of the protein sequence of isoform b is shown in Table 3 as SEQ ID NO: 25.

### Nucleic Acid Encoding Human FVIII Polypeptide

The 18 specific identified codon optimized nucleic acids encoding human FVIII polypeptide are referred to herein as F8QQ1-F8QQ18, as shown in Table 3 (SEQ ID NO: 1-18). Surprisingly there were variations in activity among the 18 codon-optimized sequences (see FIG. 2). In one embodiment, the codon optimized FVIII sequence has the nucleotide sequence set forth in one of SEQ ID NO: 1-18. In one embodiment, the codon-optimized FVIII sequence has the nucleotide sequence set forth in one of SEQ ID NOs 1, 2, 4, 5, 7-9, 11-15 or 18. In one embodiment, the codon-optimized FVIII sequence has the nucleotide sequence set forth in one of SEQ ID NOs 4, 5, 7, 12-15 or 18. In one embodiment, the codon-optimized FVIII sequence has the nucleotide sequence set forth in one Of SEQ ID NOs 4, 5, 13, or 15. In one embodiment, the codon-optimized FVIII sequence has the nucleotide sequence set forth in one of SEQ ID NOs 4 or 5. Minor changes to the nucleotide sequence are not expected to appreciably alter the activity of the identified nucleic acids. Such sequence changes may be silent changes (not resulting in amino acid changes in the encoded protein) or alterntively may lead to amino acid substitutions and as such code for variants of the human FVIII polypeptide of SEQ ID NO: 19. Non-limiting examples of such polypeptide variants are described herein.

In some embodiments of the compositions and methods disclosed herein the codon optimized nucleic acid has a nucleotide sequence that is at least 90%, at least 91%, at least 92%, at least 93%, at least 94%, at least 95%, at least 96%, at least 97%, at least 98%, at least 99% or more similar to the sequence set forth in one of SEQ ID NO: 1-18.

In some embodiments of the compositions and methods described herein, the human FVIII polypeptide has the amino acid sequence set forth below (SEQ ID NO: 19), or is at least 90%, at least 91%, at least 92%, at least 93%, at least 94%, at least 95%, at least 96%, at least 97%, at least 98%, at least 99% or more identical thereto.

(SEQ ID NO: 19)
MQIELSTCFFLCLLRFCFSATRRYYLGAVELSWDYMQSDLGELPVDARFP
PRVPKSFPENTSVVYKKTLFVEFTDHLFNIAKPRPPWMGLLGPTIQAEVY
DTVVITLKNMASHPVSLHAVGVSYWKASEGAEYDDQTSQREKEDDKVFPG
GSHTYVWQVLKENGPMASDPLCLTYSYLSHVDLVKDLNSGLIGALLVCRE
GSLAKEKTQTLHKFILLFAVEDEGKSWHSETKNSLMQDRDAASARAWPKM
HTVNGYVNRSLPGLIGCHRKSVYWHVIGMGTTPEVHSIFLEGHTFLVRNH
RQASLEISPITFLTAQTLLMDLGQFLLFCHISSHQHDGMEAYVKVDSCPE
EPQLQMKNNEEAEDYDDDLTDSEMDVVRFDDDNSPSFIQIRSVAKKHPKT
WVHYIAAEEEDWDYAPLVLAPDDRSYKSQYLNNGPORIGRKYKKVREMAY
TDETFKTREAIQHESGILGPLLYGEVGDTLLIIFKNQASRPYNIYPHGIT
DVRPLYSRRLPKGVKHLKDFPILPGEIFKYKWTVTVEDGPTKSDPRCLTR
YYSSFVNMERDLASGLIGPLLICYKESVDQQGNQIMSDKRNVILESVEDE
NRSWYLTENIQRFLPNPAGVOLEDPEFQASNIMHSINGYVFDSLOLSVCL
HEVAYWYILSIGAQTDFLSVFFSGYTFKHKMVYEDTLTLFPFSGETVFMS
MENPGLWILGCHNSDFRNRGMTALLKVSSCDKNTGDYYEDSYEDISAYLL
SKNNAIEPRSFSQNPPVLKRHOREITRTTLOSDQEEIDYDDTISVEMKKE
DEDIYDEDENQSPRSFQKKTRHYFIAAVERLWDYGMSSSPHVLRNRAQSG
SVPQFKKVVFQEFTDGSFTQPLYRGELNEHLGLLGPYIRAEVEDNIMVTF
RNQASRPYSFYSSLISYEEDOROGAEPRKNFVKPNETKTYFWKVQHHMAP
TKDEFDCKAWAYFSDVDLEKDVHSGLIGPLLVCHTNTLNPAHGRQVTVQE
FALFFTIFDETKSWYFTENMERNCRAPCNIQMEDPTFKENYRFHAINGYI
MDTLPGLVMAQDQRIRWYLLSMGSNENIHSIHFSGHVFTVRKKEEYKMAL
YNLYPGVFETVEMLPSKAGIWRVECLIGEHLHAGMSTLFLVYSNKCQTPL
GMASGHIRDFQITASGQYGQWAPKLARLHYSGSINAWSTKEPFSWIKVDL
LAPMIIHGIKTQGARQKFSSLYISQFIIMYSLDGKKWOTYRGNSTGTLMV
FFGNVDSSGIKHNIENPPIIARYIRLHPTHYSIRSTLRMELMGCDLNSCS
MPLGMESKAISDAQITASSYFTNMFATWSPSKARLHLOGRSNAWRPQVNN
PKEWLQVDFQKTMKVTGVTTQGVKSLLTSMYVKEFLISSSQDGHQWTLFF
QNGKVKVFQGNQDSFTPVVNSLDPPLLTRYLRIHPQSWVHQIALRMEVLG
CEAQDLY

In some embodiments, the first 19 amino acids of the human FVIII polypeptide (e.g., shown underlined above in SEQ ID NO: 19) is an N-terminal secretory signal sequence (otherwise referred to as the signal sequence, signal peptide) with the amino acid sequence MQIELSTCFFLCLLRFCFS (SEQ ID NO: 20). In one embodiment, the FVIII polypeptide sequence does not contain the N-terminal signal sequence. In one embodiment, the FVIII polypeptide has a different secretory signal sequence. For example, one or more amino acids are modified (substituted, deleted, or inserted) to create a functional variant, or the entire sequence is replaced by a different amino acid that serve as a secretory sequence. In one embodiment, the FVIII polypeptide entirely lacks a signal sequence. In such embodiments, the codon-optimized nuleic acid will have the nucleotide sequence of one of SEQ ID NO: 1-18, further lacking the first 19 codons (the 5′ most 57 nucleotides) that encode the N-terminal signal sequence. In one embodiment, the human FVIII polypeptide further contains a heterologous signal sequence that promotes secretion from the liver, in place of the native signal sequence. In one embodiment, the heterologous secretory signal peptide is a signal peptide with the amino acid sequence set forth in Table 3, or a functional variant thereof. Non-limiting examples of heterologous signal peptides are disclosed herein, including, but not limited to, signal peptides comprising the amino acid sequence of any of SEQ ID NO: 60-71 and 77-78, or any signal sequence shown in Table 3, or any signal sequence disclosed in U.S. Pat. Nos. 9,873,868, and 7,071,172; which are incorporated herein by reference. In one embodiment, the heterologous signal sequence is BM40 as described in Holden et al., 2005, VOLUME 280, ISSUE 17, P17172-17179, which is incorporated by reference in entirety. Nucleotide coding sequences for such signal peptides are shown in Table 3. In such embodiments, the codon optimized nucleic acid will lack the 5′ most 57 nucleotides that encode the native N-terminal signal sequence, and instead have a nucleotide sequence that encodes the heterologous signal sequence. Nucleotide sequences coding for such signal peptides are shown in Table 3 and further discussed herein.

The administration of the expression vector containing the codon-optimized nucleic acid described herein leads to increased expression of the FVIII polypeptide in a subject, as compared to the expression resulting from administration of an otherwise identical expression vector containing a non-codon optimized (native) nucleic acid encoding the same FVIII polypeptide. Such expression can be measured by the amount of the expressed polypeptide or by the activity of the polypeptide. In some embodiments, increased expression refers to at least 25% greater exogenous FVIII polypeptide or activity in the blood of an animal administered the codon-optimized FVIII nucleic acid, as compared to the level resulting from the native FVIII nucleic acid sequence. In some embodiments, increased expression refers to at least 50% greater, at least 75% greater, at least 100% greater, at least 3-fold greater, at least 4-fold greater, at least 5-fold greater, at least 6-fold greater, at least 7-fold greater, at least 8-fold greater, at least 9-fold greater, at least 10-fold greater, at least 15-fold greater, at least 20-fold greater, at least 25-fold greater, at least 30-fold greater, at least 40-fold greater, at least 50-fold greater, at least 60-fold greater, at least 70-fold greater, at least 80-fold greater, at least 90-fold greater, at least 100-fold greater, at least 125-fold greater, at least 150-fold greater, at least 175-fold greater, at least 200-fold greater, at least 225-fold greater, or at least 250-fold greater exogenous FVIII polypeptide or activity in the blood of an animal administered the codon-optimized nucleic acid encoding FVIII polypeptide, as compared to the level of exogenous Factor VIII polypeptide or activity in the blood of an animal administered the native FVIII encoding nucleic acid.

Optimized rAAV Vector Genome

In some embodiments of the methods and compositions as disclosed herein, an optimized rAAV vector genome is created from any of the elements disclosed herein and in any combination, including nucleic acid sequences encoding a promoter, an ITR, a poly-A tail, elements capable of increasing or decreasing expression of a heterologous gene, and in one embodiment, a nucleic acid sequence that is codon optimized for expression of FVIII protein in vivo (i.e., coFVIII or codon optimized FVIII) and optionally, one or more element to reduce immunogenicity. Such an optimized rAAV vector genome can be used with any AAV capsid that has tropism for the tissue and cells in which the rAAV vector genome is to be transduced and expressed.

In some embodiments, rAAV genome lacks the AAV P5 promoter or, a fragment thereof, which is normally located upstream of the promoter (e.g., liver-specific promoter) as disclosed herein. Normally, the P5 promoter controls expression of the AAV rep/cap proteins during AAV replication. In some embodiments, this P5 promoter fragment is present in the rAAV vector as disclosed herein which contains predicted transcription factor binding sites, e.g., cyclic AMP-responsive element-binding protein 3 (CREB3), which can be activated by endoplasmic reticulum (ER)/Golgi stress (Sampieri 2019), activating transcription factor 2 (ATF2), which is also involved in stress response (Watson 2017), Nuclear Receptor Subfamily 1 Group I Member 2 (NR1I2) (also known as Pregnane X receptor [PXR]) is known to be enriched in liver, and is activated by pregnane steroids, rifampin and other molecules including dexamethasone (NR1I2_HGNC) (Xing 2020). Accordingly, in some embodiments, a fragment of the AAV P5 promoter in the rAAV genome is removed without affecting the intended performance of the FVIII cassette. In some embodiments, the rAAV vector also comprises an RNA polymerase II termination sequence located between the polyA signal and the 3′ ITR. An exemplary terminal sequence is SEQ ID NO: 45, or SEQ ID NO:465, the later of which introduces two termination codons and one restriction site (e.g., XhoI) replaces TAG, and is located immediately downstream of the last coding amino acids of hFVIII, and immediately located upstream of the 3′ UTR.

### Liver Specific Promoters (LSP)

In some embodiments, of the nucleic acid sequences, AAV vectors, constructs and methods to treat Hemophilia A described herein, to achieve appropriate levels of FVIII expression, the codon-optimized nucleic acid is operatively linked to a liver specific promoter (LSP). A LSP enables expression of the operatively linked gene in the liver, and can in some embodiments, be an inducible LSP. In an embodiment, a LSP is located upstream 5′ and is operatively linked to the heterologous nucleic acid sequence encoding the FVIII polypeptide. Exemplary liver-specific promoters are disclosed herein.

In some embodiments of the nucleic acid sequences, AAV vectors, constructs and methods to treat Hemophilia A described herein, the liver-specific promoter is any liver-specific promoter disclosed WO2020102645 and WO2021102107, where the LSP has been improved. For example, a liver specific promoter useful in the rAAV vectors as disclosed herein is any LSP disclosed International Patent Application numbers WO2020102645 and WO2021102107 which has been modified to replace the the sequence of SEQ ID NO: 450 (corresponding to as SEQ ID NO: 126 in WO2021102107 or referred to as CRE0052 or LVR_CRE_0052_G6PC sequence) in any of the LSP sequences in WO2021102107 with a sequence selected from SEQ ID NO: 40 or 41, or a sequence having at least 85%, 90%, 95%, 96%, 97%, 98% or 99% nucleotide sequence identity thereto. Using SP131A1 (or LVR131_A1) promoter as an exemplary promoter, which is disclosed as SEQ ID NO: 94 in WO2021102107, in the current application the promoter has been modified to replace SEQ ID NO 450 (corresponding to SEQ ID NO: 126 in WO2021102107) with SEQ ID NO: 40 or 41, or a sequence having at least 85%, 90%, 95%, 96%, 97%, 98% or 99% nucleotide sequence identity thereto. Any promoter disclosed in WO2021102107 is encompased for use herein, wherein if the promoter comprises SEQ ID NO 450 (corresponding to SEQ ID NO: 126 in WO2021102107), it can be replaced with SEQ ID NO: 40 or 41, or a sequence having at least 85%, 90%, 95%, 96%, 97%, 98% or 99% nucleotide sequence identity thereto.

In some embodiments of the nucleic acid sequences, AAV vectors, constructs and methods to treat Hemophilia A described herein, the promoter is a LP1 promoter (SEQ ID NO: 432), or a variant having at least sequence at least 85%, 90%, 95%, 96%, 97%, 98% or 99% nucleotide sequence identity thereto.

In some embodiments of the nucleic acid sequences, AAV vectors, constructs and methods to treat Hemophilia A described herein, the promoter is an inducible promoter or, a variant thereof as described in International Application No. PCT/GB2020/050107, which is incorporated herein by reference.

In some embodiments of the nucleic acid sequences, AAV vectors, constructs and methods to treat Hemophilia A described herein, a synthetic liver-specific promoter useful in the AAV vector is any LSP promoter selected from SEQ ID NOS: 86, 88, 91-96, 106, 146-150, 439-441, or 481-500 as disclosed herein, or any LSP selected from SEQ ID NO: 270-341 or 342-430 as disclosed herein, or a synthetic liver-specific promoter thereof which is able to promote liver-specific transgene expression and has an activity in liver cells which is at least 15%, 20%, 30%, 40%, 50%, 60%, 70%, 80%, 90%, 100%, 125%, 150%, 175%, 200%, 250%, 300%, 350% or 400% of the activity of the TTR promoter comprising SEQ ID NO: 431 as International Application WO2021102107, or a synthetic promoter which is disclosed in Table 4 of International Application WO2021102107, which is incorporated herein in its entirety by reference.

In some embodiments of the nucleic acid sequences, AAV vectors, constructs and methods to treat Hemophilia A described herein, a synthetic liver specific promoter is selected from any of: SEQ ID NOS: 86, 88, 91-96, 106, 146-150, or 270-430 as disclosed herein, or nucleic acid sequence that is at least 80%, or at least 90% or 95% identical thereto or to the source regulatory nucleic acid sequence.

In some embodiments of the nucleic acid sequences, AAV vectors, constructs and methods to treat Hemophilia A disclosed herein, a liver-specific promoter (LSP) in a AAV expressing a FVIII polypeptide as disclosed herein and useful in the methods to treat Hemophilia A as disclosed herein comprises a nucleic acid sequence selected from any promoter listed from SEQ ID NOS: 86 (CRM 0412), SEQ ID NO: 91 (SP0412) or SEQ ID NO: 92 (SP0422), SEQ ID NOS: 93 (SP0239), SEQ ID NO: 94 (SP0265), SEQ ID NO: 95 (SP0240) or SEQ ID NO: 96 (SP0246), SEQ ID NO: 106 (HSP) or SEQ ID NO: 146 (SP0265-UTR), SEQ ID NO: 147 (SP0239-UTR), SEQ ID NO: 148 (SP0240-UTR), SEQ ID NO: 149 (SP0246-UTR) or SEQ ID NO: 150 (SP0131-A1-UTR), SEQ ID NO: 439 (LVR 0243); SEQ ID NO: 440 (LVR 0412) and SEQ ID NO: 441 (A1 Promoter), as disclosed herein, or a functional fragment or variant of any LSP selected from SEQ ID NO: 270-341 or 342-430, or a functional fragment or variant thereof of SEQ ID NOS: 86, 88, 91-96, or 146-150, 439-441, 270-430, or 481-500, as disclosed herein. In some embodiments, the synthetic liver-specific promoter is able to promote liver-specific transgene expression and has an activity in liver cells which is at least 15%, 20%, 30%, 40%, 50%, 60%, 70%, 80%, 90%, 100%, 125%, 150%, 175%, 200%, 250%, 300%, 350% or 400% of the activity of the TBG promoter of SEQ ID NO: 435.

In some embodiments of the nucleic acid sequences, AAV vectors, constructs and methods to treat Hemophilia A disclosed herein, a synthetic liver-specific promoter is used, such as selected from any or any LSP promoter selected from any of SEQ ID NO: 97, SEQ ID NO: 98 or SEQ ID NO: 99, or a variant having at least sequence at least 85%, 90%, 95%, 96%, 97%, 98% or 99% nucleotide sequence identity thereto. In some embodiments, a synthetic liver-specific promoter is selected from any of SEQ ID NO: 97, SEQ ID NO: 98 or SEQ ID NO: 99, or a functional variant having at least sequence at least 85%, 90%, 95%, 96%, 97%, 98% or 99% nucleotide sequence identity thereto, where the synthetic liver-specific promoter is able to promote liver-specific transgene expression and has an activity in liver cells which is at least 15%, 20%, 30%, 40%, 50%, 60%, 70%, 80%, 90%, 100%, 125%, 150%, 175%, 200%, 250%, 300%, 350% or 400% of the activity of the TBG promoter of SEQ ID NO: 435. In some embodiments, the liver specific promoter comprising a sequence of SEQ ID NO: 97, 98 or 99, or a functional variant thereof have at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more identity to SEQ ID NO: 99 is used.

In some embodiments of the nucleic acid sequences, AAV vectors, constructs and methods to treat Hemophilia A disclosed herein, a synthetic liver-specific promoter is used, such as selected from a liver-specific promoter selected from any of SEQ ID NO: 481-500 (for example, promoters SP0246, SP0412, SP0472, SP0380, SP0381, SP0409, and SP0411), or a variant having a sequence of at least 85%, 90%, 95%, 96%, 97%, 98% or 99% nucleotide sequence identity thereto. In some embodiments, a synthetic liver-specific promoter is selected from any of SEQ ID NO: 481-500, or a functional variant having at least sequence at least 85%, 90%, 95%, 96%, 97%, 98% or 99% nucleotide sequence identity thereto, where the synthetic liver-specific promoter is able to promote liver-specific transgene expression and has an activity in liver cells which is at least 15%, 20%, 30%, 40%, 50%, 60%, 70%, 80%, 90%, 100%, 125%, 150%, 175%, 200%, 250%, 300%, 350% or 400% of the activity of the TBG promoter of SEQ ID NO: 435. Synthetic liver-specific promoters SP0246 and SP0412 are further described in International Patent Application number WO2021/102107, the contents of which are incorporated herein by reference.

In one embodiment, the liver-specific promoter is any of the liver-specific promoters disclosed in WO2021/102107, WO 2020/102645, or WO2020/102667, the contents of each of which are incorporated herein by reference.

In one embodiment, the liver-specific promoter is a liver specific promoter having 80% sequence identity to any of the liver-specific promoters disclosed in WO2021/102107, WO 2020/102645, or WO2020/102667, the contents of each of which are incorporated herein by reference.

Any of the liver-specific promoters disclosed in WO2021/102107, WO 2020/102645, or WO2020/102667 can be operatively linked to a codon-optimized FVIII transgene described herein to promote its expression, e.g., in the liver.

In one embodiment, the synthetic liver-specific promoters comprise or consist of CRE0042, or a functional variant thereof, operably linked to CRE0073, or a functional variant thereof.

CRE0042 is a cis-regulatory element. It functions in combination with a promoter element to modulate, typically enhance, liver-specific transcription from the promoter in which they are comprised.

CRE0073 is a promoter element (minimal or proximal promoter). It functions in combination with one or more CREs to provide liver-specific transcription from the promoter in which they are comprised.

Promoter element CRE0073 can be contiguous (i.e., positioned immediately adjacent to one another) with the adjacent cis-regulatory element CRE0042, or it can be separated by a spacer or other sequence. CRE0042 operably linked to CRE0073 has been found to provide high level of liver-specific expression.

Additional CRE elements are further described in International Patent Application number WO2021/102107, the contents of which are incorporated herein by reference.

In some embodiments of the present invention, the synthetic liver-specific promoter comprises or consists of promoter SP0472 (SEQ ID NO: 483), or a functional variant thereof. Suitably the functional variant of the synthetic liver-specific promoter comprises a sequence that is at least 70% identical to the reference synthetic liver-specific promoter, more preferably at least 80%, 90%, 91%, 92%, 93%, 94%, 95%, 96%, 97%, 98% or 99% identical to the reference synthetic liver-specific promoter.

In some embodiments of the present invention the synthetic liver-specific promoter has a length of 350 or less nucleotides, preferably 340 or less nucleotides, more preferably 330 or less nucleotides, most preferably 320 or less nucleotides. In some embodiments of the present invention the synthetic liver-specific promoter has a length of 310 or less nucleotides, preferably 300 or less nucleotides, more preferably 290 or less nucleotides, most preferably 280 or less nucleotides. In some preferred embodiments of the present invention the synthetic liver-specific promoter has a length of 270 or less nucleotides.

In a further aspect of the invention, there is provided an expression cassette comprising SP0472, or a functional variant thereof, operably linked to a sequence encoding an expression product, suitably a gene, e.g., a transgene.

In a further aspect of the invention, there is provided an expression cassette containing any of the codon-optimized nucleic acid described herein, operably linked to a liver-specific promoter.

The liver-specific promoter can be any promoter. In one embodiment, the promoter can be any of the above described promoters.

In one embodiment, the codon-optimized nucleic acid, which can be part of an expression vector, can be operably linked to a liver-specific promoter, wherein the liver specific promoter is selected from any of: SEQ ID NOS: 481-500 or a functional fragment therein, or a liver specific promoter having at least 80% sequence identity to SEQ ID NOs: 481-500.

In a further aspect of the invention, there is provided an expression cassette containing any of the codon-optimized nucleic acid described herein, operably linked to a liver-specific promoter, wherein the liver specific promoter is selected from any of: SEQ ID NOS: 481-483 or a functional fragment thereof, or a liver specific promoter having at least 80% sequence identity to SEQ ID NOs: 481-483.

In some embodiments, the expression product is a blot-clotting protein, such as FVIII or FIX or variant thereof. In some embodiments, the expression product is FVIII protein. In some embodiments, the transgene in a codon optimized FVIII gene. In some embodiments, a codon optimized FVIII gene is a codon optimized FVII-QQ gene.

In a further aspect, there is provided a vector comprising a synthetic liver-specific promoter or an expression cassette according to the present invention. In some embodiments the vector is an expression vector. In some embodiments the vector is a viral vector. In some embodiments the vector is a gene therapy vector, suitably an AAV vector, an adenoviral vector, a retroviral vector, or a lentiviral vector. AAV vectors are of particular interest.

In a further aspect, there is provided a virion (viral particle) comprising a vector, suitably a viral vector, according to the present invention.

In a further aspect, there is provided a pharmaceutical composition comprising a synthetic liver-specific promoter, expression cassette, vector, or virion according to the present invention.

In a further aspect, there is provided a synthetic liver-specific promoter, expression cassette, vector, virion or pharmaceutical composition according to the present invention for use in therapy, i.e., the prevention or treatment of a medical condition or disease. Suitably the condition or disease is associated with aberrant gene expression, optionally aberrant gene expression in the liver. Suitably the use is for gene therapy, preferably for use in treatment of a disease involving aberrant gene expression.

In some embodiments, the disease is Pompe disease or hemophilia.

In a further aspect, there is provided a cell comprising a synthetic liver-specific promoter, expression cassette, vector, or virion as described herein. In some embodiments the cell is a eukaryotic cell, optionally a mammalian cell, optionally a human cell. Suitably the cell can be a liver cell, optionally wherein the cell is a human liver cell. The synthetic liver-specific promoter or expression cassette can be in a vector or can be in the genome of the cell.

In a further aspect, there is provided a synthetic liver-specific promoter, expression cassette, vector, virion or pharmaceutical composition as described herein for use in the manufacture of a pharmaceutical composition for the treatment of a medical condition or disease as discussed herein. In some embodiments, the disease is Pompe disease. In some preferred embodiments, the synthetic liver-specific promoter, expression cassette, vector, virion, or pharmaceutical composition as described herein are for use in the manufacture of a pharmaceutical composition for the treatment of haemophilia A.

In a further aspect, there is provided a method for producing an expression product, the method comprising providing a synthetic liver-specific expression cassette of the present invention in a liver cell and expressing the gene present in the synthetic liver-specific expression cassette. The method can be in vitro or ex vivo, or it can be in vivo. In some embodiments the method is a bioprocessing method. In some preferred embodiments, the expression product is Factor VIII protein.

In a further aspect, there is provided a method of expressing a therapeutic transgene in a liver cell, the method comprising introducing into the liver cell a synthetic liver-specific expression cassette, vector or virion as described herein. In some preferred embodiments, the therapeutic transgene is the Factor VIII gene.

In some embodiments, a liver-specific promoter described herein, e.g., SP0472, is operatively linked to a transgene that encodes a therapeutic expression product, preferably a therapeutic polypeptide suitable for use in treating a disease or condition associated with aberrant gene expression in the liver. The therapeutic expression product can be a protein, e.g., a secretable protein such as, e.g., a clotting factor (e.g., factor IX or factor VIII), a cytokine, a growth factor, an antibody or nanobody, a chemokine, a plasma factor, insulin, erythropoietin, lipoprotein lipase, or a toxic protein. In some embodiments, the protein is a secretable protein. In some embodiments, the secretable protein may act on the liver. In some embodiments, the secretable protein may act on tissues other than liver (e.g., muscle, CNS, kidney, etc.). In some embodiments the secretable protein may act on the liver and tissues other than liver. Alternatively, the therapeutic expression product may be RNA, such as an siRNA or miRNA. A non-exhaustive list of therapeutic expression products (and sequences encoding them) envisaged for use in the present invention includes: factor VIII, factor IX, factor VII, factor X, von Willebrand factor, erythropoietin (EPO), interferon-a, interferon-B, interferon-y, interleukin 1 (IL-1), interleukin 2 (IL-2), interleukin 3 (IL-3), interleukin 4 (IL-4), interleukin 5 (IL-5), interleukin 6 (IL-6), interleukin 7 (IL-7), interleukin 8 (IL-8), interleukin 9 (IL-9), interleukin 10 (IL-10), interleukin 11 (IL-11), interleukin 12 (IL-12), chemokine (C-X-C motif), ligand 5 (CXCL5), granulocyte-colony stimulating factor (G-CSF), granulocyte-macrophage colony stimulating factor (GM-CSF), macrophage colony stimulating factor (M-CSF), stem cell factor (SCF), keratinocyte growth factor (KGF), monocyte chemoattractant protein-1 (MCP-1), tumour necrosis factor (TNF), afamin (AFM), acid alfa glucosidase (GAA), a1-antitrypsin, a-galactosidase A, α-L-iduronidase, ATP7b, ornithine transcarbamoylase, phenylalanine hydroxylase, lipoprotein lipase, aromatic amino acid decarboxylase (AADC), ATPase Sarcoplasmic/Endoplasmic Reticulum Ca2+ Transporting 2 (ATP2A2), cystic fibrosis transmembrane conductance regulator (CTFR), glutamic acid decarboxylase 65 kDa protein (GAD65), glutamic acid decarboxylase 67 kDa protein (GAD67), lipoprotein lipase (LPL), nerve growth factor (NGF), neurturin (NTN), porphobilinogen deaminase (PBGD), sarcoglycan alpha (SGCA), soluble fins-like tyrosine kinase-1 (sFLT-1), apoliproteins, low-density lipoprotein receptor (LDL-R), albumin, glucose-6-phosphatase, antibodies, nanobodies, aptamers, anti-viral dominant-negative proteins, and functional fragments, subunits or mutants thereof.

In a further aspect, there is provided a method of therapy of a subject, preferably a human, in need thereof, the method comprising:
administering to the subject an expression cassette, vector, virion, or pharmaceutical composition as described herein, which comprises a sequence encoding a therapeutic product operably linked to a promoter according to the present invention; and
expressing a therapeutic amount of the therapeutic product in the liver of said subject.

In some preferred embodiments, the therapeutic product is Factor VIII.

In some embodiments the method further comprises introducing into the liver of the subject an expression cassette, vector, virion, or pharmaceutical composition as described herein, which comprises a gene encoding a therapeutic product. In some preferred embodiments the vector is a viral gene therapy vector, preferably an AAV vector.

In some embodiments of the invention, the functional variant of CRE0042 comprises the sequence: GTTCAAACATG-Na-CTAATACTCTG-Nb-TGCAAGGGTCAT-Nc-TTACTCAACA (SEQ ID NO: 504) or a sequence that is at least 70%, 80%, 90%, 95% or 99% identical thereto, wherein Na, Nb and Nc represent optional spacer sequences. When present, Na optionally has a length of from 1 to 10 nucleotides, preferably from 1 to 5 nucleotides, and more preferably 2 nucleotides. When present, Nb optionally has a length of from 1 to 10 nucleotides, preferably from 2 to 6 nucleotides, and more preferably 4 nucleotides. When present, Nc optionally has a length of from 8 to 23 nucleotides, preferably from 10 to 20 nucleotides, and more preferably 15 nucleotides.

Activity in a functional variant can be assessed by comparing expression of a suitable reporter under the control of the reference synthetic liver-specific promoter with the putative functional variant under equivalent conditions. Suitable assays for assessing liver-specific promoter activity are disclosed herein, e.g., in Examples 2 and 3.

Functional variants of a given synthetic liver-specific promoter can comprise functional variants of one or more CREs and/or functional variants of the promoter element present in the reference synthetic liver-specific promoter. Functional variants of SP0472 can comprise functional variants of CRE0042 and/or functional variants of CRE0073.

Functional variants of a given synthetic liver-specific promoter can comprise one or more additional CREs to those present in a reference synthetic liver-specific promoter. The additional CREs can be CREs disclosed herein, or they can be other CREs.

Functional variants of a given synthetic liver-specific promoter can comprise one or more additional regulatory elements compared to a reference synthetic liver-specific promoter. For example, they may comprise an inducible elements, an intronic element, a boundary control element, an insulator, a locus control region, a response element, a binding site, a segment of a terminal repeat, a responsive site, a stabilizing element, a de-stabilizing element, and a splicing element, etc., provided that they do not render the promoter substantially non-functional. Functional variants can also include a 5′ UTR sequence.

In one embodiment of the nucleic acid sequences, AAV vectors, constructs and methods to treat Hemophilia A disclosed herein, the liver promoter is a promoter that has some expression in the liver. In one embodiment, the promoter that has some expression in the liver is the M2 liver promoter comprising a sequence of SEQ ID NO: 98, or a functional variant have at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more identity to SEQ ID NO: 98.

In some embodiments of the nucleic acid sequences, AAV vectors, constructs and methods to treat Hemophilia A disclosed herein, the synthetic liver specific promoter comprises SEQ ID NO: 99, or nucleic acid sequence that is at least 50%, preferably 60%, 70%, 80%, 90% or 95% identical to the source regulatory nucleic acid sequence. In some embodiments, a synthetic liver specific promoter comprises SEQ ID NO: 99, or nucleic acid sequence that is at least 80%, or at least 90% or 95% identical to nucleotides 1-26 of SEQ ID NO: 99.

In some embodiments of the nucleic acid sequences, AAV vectors, constructs and methods to treat Hemophilia A disclosed herein, a synthetic liver specific promoter that is at least 50%, 60%, 70%, 80%, 90% or 95% identical to SEQ ID NO: 99 comprises a nucleic acid sequence where 2% or 1% or fewer of the nucleotides of SEQ ID NO: 99 are altered. In some embodiments, a synthetic liver-specific promoter useful in the methods and compositions as disclosed herein is the same length, or not substantially altered, or 1, 2, 3, 4, 5, or 6 nucleotides longer or 1, 2, 3, 4, 5, or 6 shorter than the length of SEQ ID NO: 99.

In some embodiments of the nucleic acid sequences, AAV vectors, constructs and methods to treat Hemophilia A disclosed herein, no nucleotides have been deleted when compared to SEQ ID NO: 99. In some embodiments, no nucleotides are inserted when compared to SEQ ID NO: 99. In some embodiments, all modifications made to SEQ ID NO: 99 are nucleotide substitutions.

In some embodiments of the nucleic acid sequences, AAV vectors, constructs and methods to treat Hemophilia A disclosed herein, a synthetic liver specific promoter that is at least 50%, 60%, 70%, 80%, 90% or 95% identical to SEQ ID NO: 99 comprises a source regulatory nucleic acid sequence which is active in liver, and the second type of cell or tissue is muscle; or a source regulatory nucleic acid sequence which is active in liver, and the second type of cell or tissue is CNS; or a source regulatory nucleic acid sequence which is active in muscle, and the second type of cell or tissue is liver; or a source regulatory nucleic acid sequence which is active in muscle, and the second type of cell or tissue is CNS.

In some embodiments, a liver-specific promoter which is a functional variant of a given promoter element preferably retains at least 80% of its activity, more preferably at least 90% of its activity, more preferably at least 95% of its activity, and yet more preferably 100% of its activity (compared to the reference promoter comprising the unmodified promoter element). Suitable assays for assessing liver-specific promoter activity are disclosed in Examples 12 and 13 of International Application WO2021102107 which is incorporated herein in its entirety by reference.

In some embodiments of the nucleic acid sequences, AAV vectors, constructs and methods to treat Hemophilia A disclosed herein, liver specific promoters include, but are not limited to, transthyretin promoter (TTR), LSP promoter (LSP), a synthetic liver specific promoter. For example, in some embodiments of the methods and compositions as disclosed herein, the promoter is a liver specific promoter (LSP), and can be selected from any liver specific promoters including, but not limited to, a transthyretin promoter (TTR), a Liver specific promoter (LSP), for example, as disclosed in U.S. Pat. No. 5,863,541 (TTR promoter), or LSP promoter (PNAS; 96: 3906-3910, 1999. See e.g., p. 3906, Materials and Methods, rAAV construction), a synthetic liver promoter, the references which are incorporated herein in their entireties by reference. Other liver promoters can be used, for example, synthetic liver promoters.

In some embodiments, the TTR promoter is a truncated TTR promoter, e.g., comprising SEQ ID NO: 431, or SEQ ID NO: 12 as disclosed in International WO 2020102645, which is incorporated herein in its entirety by reference, or a functional variant having at least sequence at least 85%, 90%, 95%, 96%, 97%, 98% or 99% nucleotide sequence identity thereto. In some embodiments, the LSP is a TBG promoter, e.g., comprising SEQ ID NO: 435, or a functional variant having at least sequence at least 85%, 90%, 95%, 96%, 97%, 98% or 99% nucleotide sequence identity thereto.

Other liver specific promoters include, but are not limited to promoters for the LDL receptor, Factor VIII, Factor IX, phenylalanine hydroxylase (PAH), ornithine transcarbamylase (OTC), and a 1-antitrypsin (hAAT), and HCB promoter. Other liver specific promoters include the AFP (alpha fetal protein) gene promoter and the albumin gene promoter, as disclosed in EP Patent Publication 0 415 731, the a-1 antitrypsin gene promoter, as disclosed in Rettenger, Proc. Natl. Acad. Sci. 91 (1994) 1460-1464, the fibrinogen gene promoter, the APO-A1 (Apolipoprotein A1) gene promoter, and the promoter genes for liver transference enzymes such as, for example, SGOT, SGPT and g-glutamyle transferase. See also 2001/0051611 and PCT Patent Publications WO 90/07936 and WO 91/02805, which are incorporated herein in their entirety by reference. In some embodiments, the liver specific promoter is a recombinant liver specific promoter, e.g., as disclosed in US20170326256A1, which is incorporated herein in its entirety by reference.

In some embodiments, a liver specific promoter is the hepatitis B X-gene promoter and the hepatitis B core protein promoter. In some embodiments, liver specific promoters can be used with their respective enhancers. The enhancer element can be linked at either the 5′ or the 3′ end of the nucleic acid encoding the FVIII polypeptide. The hepatitis B X gene promoter and its enhancer can be obtained from the viral genome as a 332 base pair EcoRV-NcoI DNA fragment employing the methods described in Twu, J Virol. 61 (1987) 3448-3453. The hepatitis B core protein promoter can be obtained from the viral genome as a 584 base pair BamHI-Bglll DNA fragment employing the methods described in Gerlach, Virol 189 (1992) 59-66. It may be necessary to remove the negative regulatory sequence in the BamHI-Bglll fragment prior to inserting it.

### UTRs, Regulatory Sequences, and Intron Sequences

In some embodiments of the nucleic acid sequences, AAV vectors, constructs and methods to treat Hemophilia A disclosed herein, the promoter, i.e., the liver-specific promoter, as set out above is operably linked to one or more additional regulatory sequences. An additional regulatory sequence can, for example, enhance expression compared to the promoter which is not operably linked the additional regulatory sequence. Generally, it is preferred that the additional regulatory sequence does not substantively reduce the specificity of the liver-specific promoter.

For example, the promoter can be operably linked to a sequence encoding a UTR (e.g., a 5′ and/or 3′ UTR), an intron, or such. In some embodiments, the promoter is operably linked to sequence encoding a UTR, e.g., a 5′ UTR. A 5′ UTR can contain various elements that can regulate gene expression. The 5′ UTR in a natural gene begins at the transcription start site and ends one nucleotide before the start codon of the coding region. It should be noted that 5′ UTRs as referred to herein may be an entire naturally occurring 5′ UTR or it may be a portion of a naturally occurring 5′ UTR. The 5′ UTR can also be partially or entirely synthetic. In eukaryotes, 5′ UTRs have a median length of approximately 150 nt, but in some cases they can be considerably longer. Regulatory sequences that can be found in 5′ UTRs are disclosed in International Application WO2021102107 which is incorporated herein in its entirety by reference.

In some embodiments, a 5-UTR sequence is located 3′ of a promoter as disclosed herein, and 5′ of the heterologous nucleic acid sequence (e.g., encoding FVIII polypeptide).

In one embodiment, an exemplary 5-UTR sequence comprises, for example, a 24 bp sequence of SEQ ID NO: 41, or a functional variant have at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more of SEQ ID NO: 41.

In one embodiment, an exemplary 5-UTR sequence comprising SEQ ID NO: 41 is the sequence of SEQ ID NO: 40, or a functional variant have at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more of SEQ ID NO: 40.

In some embodiments, the 5-UTR sequence comprises SEQ ID NO: 41 or SEQ ID NO: 40, or nucleic acid sequence that is at least 50%, preferably 60%, 70%, 80%, 90% or 95% identical to the source regulatory nucleic acid sequence. In some embodiments, a 5-UTR sequence comprises SEQ ID NO: 41 or SEQ ID NO: 40 or nucleic acid sequence that is at least 80%, or at least 90% or 95% identical to nucleotides of SEQ ID NO: 41 or SEQ ID NO: 40.

In some embodiments, a 5-UTR that is at least 50%, 60%, 70%, 80%, 90% or 95% identical to SEQ ID NO: 41 or SEQ ID NO: 40 comprises a nucleic acid sequence where 2% or 1% or fewer of the nucleotides of SEQ ID NO: 41 or SEQ ID NO: 40 are altered. In some embodiments, a 5-UTR sequence useful in the methods and compositions as disclosed herein is the same length, or not substantially altered, or 1, 2, 3, 4, 5, or 6 nucleotides longer or 1, 2, 3, 4, 5, or 6 shorter than the length of SEQ ID NO: 41 or SEQ ID NO: 40.

Introns within 5′ UTRs have been linked to regulation of gene expression and mRNA export. In some embodiments, a liver-specific promoter as set out above is operably linked to a sequence encoding a 5′ UTR derived from the CMV major immediate gene (CMV-IE gene). For example, the 5′ UTR from the CMV-IE gene suitably comprises the CMV-IE gene exon 1 and the CMV-IE gene exon 1, or portions thereof. In some cases, the promoter element may be modified in view of the linkage to the 5′ UTR, for example sequences downstream of the transcription start site (TSS) in the promoter element can be removed (e.g., replaced with the 5′ UTR).

The CMV-IE 5′UTR is described in Simari, et al, Molecular Medicine 4: 700-706, 1998 “Requirements for Enhanced Transgene Expression by Untranslated Sequences from the Human Cytomegalovirus Immediate-Early Gene”, which is incorporated herein by reference. Variants of the CMV-IE 5′ UTR sequences discussed in Simari, et al. are also set out in WO2002/031137, incorporated by reference, and the regulatory sequences disclosed therein can also be used. Other UTRs that can be used in combination with a promoter are known in the art, e.g., in Leppek, K., Das, R. & Bama, M. “Functional 5′ UTR mRNA structures in eukaryotic translation regulation and how to find them”. Nat Rev Mol Cell Biol 19, 158-174 (2018), incorporated by reference.

In some embodiments the sequence encoding the 5′ UTR comprises SEQ ID NO: 145 as disclosed herein, or a functional variant thereof. In some embodiments, functional variants may have a sequence that is at least 60%, 65%, 70%, 75%, 80%, 85%, 90%, 95%, 96%, 97%, 98%, or 99% identical thereto. SEQ ID NO: 145 as disclosed herein encodes a CMV-IE 5′ UTR.

In some embodiments the sequence encoding the 5′ UTR comprises SEQ ID NO: 446 as disclosed herein, or a functional variant thereof. In some embodiments, functional variants may have a sequence that is at least 60%, 65%, 70%, 75%, 80%, 85%, 90%, 95%, 96%, 97%, 98%, or 99% identical thereto. SEQ ID NO: 446 as disclosed herein, which is a modified CMV-IE intron sequence.

In some embodiments the 5′ UTR comprises a nucleic acid motif that functions as the protein translation initiation site, e.g., sequences that define a Kozak sequence in the mRNA produced. For example, in some embodiments, the sequence encoding the 5′ UTR comprises the sequence motif GCCACC at or near its 3′ end. Other Kozak sequences or other protein translation initiation sites can be used, as is known in the art (e.g., Marilyn Kozak, “Point Mutations Define a Sequence Flanking the AUG Initiator Codon That Modulates Translation by Eukaryotic Ribosomes” Cell, Vol. 44, 283-292, Jan. 31, 1986; Marilyn Kozak “At Least Six Nucleotides Preceding the AUG Initiator Codon Enhance Translation in Mammalian Cells” J. Mol. Rid. (1987) 196, 947-950; Marilyn Kozak “An analysis of 5′-noncoding sequences from 699 vertebrate messenger RNAs” Nucleic Acids Research. Vol. 15 (20) 1987, all of which are incorporated herein by reference). The protein translation initiation site (e.g., Kozak sequence) is preferably positioned immediately adjacent to the start codon.

In some embodiments, a sequence encoding a 5′ UTR comprises SEQ ID NO: 438 as disclosed herein, or a functional variant thereof. In some embodiments, functional variants may have a sequence that is at least 60%, 65%, 70%, 75%, 80%, 85%, 90%, 95%, 96%, 97%, 98%, or 99% identical thereto. This 5′ UTR comprises six nucleotides of GCCACC, which define a Kozak sequence at the 3′ end of the CMV-IE 5′ UTR.

In some embodiments, the rAAV expressing the codon-optimized nucleic acid encoding human FVIII polypeptides for use in the methods to treat hemophila A as disclosed herein comprises an intron sequence located 3′ of the promoter sequence and 5′ of the heterologous nucleic acid (i.e., 5′ of the nucleic acid encoding the FVIII polypeptide). Intron sequences serve to increase one or more of: mRNA stability, mRNA transport out of nucleus and/or expression and/or regulation of the expressed FVIII polypeptide. In alternative embodiments, a rAAV genotype does not comprise an intron sequence.

A synthetic promoter, e.g., a synthetic liver-specific promoter, according to the present invention can be operably linked to a sequence encoding a UTR (e.g., a 5′ and/or 3′ UTR), and/or an intron, or suchlike. In some embodiments, a synthetic promoter as set herein, is operably linked to a sequence encoding a 5′ UTR and an intron. In some embodiments, the 5′ UTR and intron is derived from the CMV major immediate gene (CMV-IE gene). The CMV-IE 5′UTR and intron is described in Simari, et al., Molecular Medicine 4: 700-706, 1998 “Requirements for Enhanced Transgene Expression by Untranslated Sequences from the Human Cytomegalovirus Immediate-Early Gene”, which is incorporated herein by reference. Variants of the CMV-IE 5′ UTR and intron sequences discussed in Simari, et al. are also set out in WO2002/031137, incorporated by reference, and the regulatory sequences disclosed therein can also be used. In some embodiments the 5′ UTR or the 5′ UTR and intron suitably comprises a nucleic acid motif that functions as the protein translation initiation site, e.g., sequences that define a Kozak sequence in the mRNA produced. For example, in some embodiments, the sequence encoding the 5′ UTR comprises the sequence motif GCCACC at or near its 3′ end. Other Kozak sequences or other protein translation initiation sites can be used, as is known in the art (e.g., Marilyn Kozak, “Point Mutations Define a Sequence Flanking the AUG Initiator Codon That Modulates Translation by Eukaryotic Ribosomes” Cell, Vol. 44, 283-292, Jan. 31, 1986; Marilyn Kozak “At Least Six Nucleotides Preceding the AUG Initiator Codon Enhance Translation in Mammalian Cells” J. Mol. Rid. (1987) 196, 947-950; Marilyn Kozak “An analysis of 5″-noncoding sequences from 699 vertebrate messenger RNAs” Nucleic Acids Research. Vol. 15 (20) 1987, all of which are incorporated herein by reference). The protein translation initiation site (e.g., Kozak sequence) is preferably positioned immediately adjacent to the start codon.

In some embodiments, any one of the promoters described herein, or variants thereof, is linked to a sequence encoding a 5′ UTR and/or a 5′UTR and an intron to provide a composite promoter. Herein, such composite promoter may be referred to simply as “composite promoters”, or in some cases simply “promoters” for brevity.

In some embodiments, the intron sequence is a MVM intron sequence, for example, but not limited to intron sequence of SEQ ID NO: 442, or nucleic acid sequence having at least 80%, 85%, 90%, 95%, 96%, 97%, 98% or 99% nucleotide sequence identity thereto.

In some embodiments, the intron sequence is a HBB2 intron sequence, for example, but not limited to and intron sequence of SEQ ID NO: 443 or SEQ ID NO: 444 or nucleic acid sequence having at least 80%, 85%, 90%, 95%, 96%, 97%, 98% or 99% nucleotide sequence identity thereto.

In some embodiments, the intron sequence is an ubiquitin C (UBC) intron sequence, e.g., intron 1 from the UBC gene, or a portion thereof, e.g., as disclosed in Bianchi et al, 2009, Gene, 448 (1); 88-101, where the intron 1 sequence of the UBC gene is 812 bp and starts at chromosomal location 124,914,586, and ends at 124,913,775. In some embodiments, the intron sequence is a UBC intron, for example, but not limited to intron sequence of SEQ ID NO: 445, or nucleic acid sequence having at least 80%, 85%, 90%, 95%, 96%, 97%, 98% or 99% nucleotide sequence identity to SEQ ID NO: 445.

In some embodiments, the rAAV genotype comprises an intron sequence selected in the group consisting of a human beta globin b2 (or HBB2) intron, a FIX intron, a chicken beta-globin intron, a CMVIE intron, a UBC intron, a HBB intron sequence, a MVM sequeocne and a SV40 intron. In some embodiments, the intron is optionally a modified intron such as a modified HBB2 intron (see, e.g., SEQ ID NO: 17 in of WO2018046774A1): a modified FIX intron (see., e.g., SEQ ID NO: 19 in WO2018046774A1), or a modified chicken beta-globin intron (e.g., see SEQ ID NO: 21 in WO2018046774A1), or modified HBB2 or FIX introns disclosed in WO2015/162302, which are incorporated herein in their entirety by reference.

### Poly a Sequences and Terminator Sequences

In some embodiments, an rAAV vector genome includes at least one poly A tail that is located 3′ and downstream from the heterologous nucleic acid gene encoding the FVIII polypeptide. Any polyA sequence can be used, including but not limited to hGH poly A, synpA polyA and the like. In some embodiments, the polyA is a synthetic polyA sequence. In some embodiments, the rAAV vector genome comprises two polyA tails, e.g., a hGH poly A sequence and another polyA sequence, where a spacer nucleic acid sequence is located between the two poly A sequences.

In some embodiments of the nucleic acid sequences, AAV vectors, constructs and methods to treat Hemophilia A disclosed herein, the polyA signal is 3′ of the heterologous nucleic acid sequence encoding the FVIII polypeptide. In some embodiments, the rAAV genome comprises 3′ of the nucleic acid encoding the FVIII polypeptide, a first polyA sequence and a reverse RNA polymerase II terminator sequence (rev RNA PolII terminator sequence), and the 3′ ITR.

In some embodiments, the rAAV genome comprises 3′ of the nucleic acid encoding the FVIII polypeptide, a first polyA sequence, a spacer nucleic acid sequence (e.g., of between 100-400 bp, or about 100-250 bp, or about 250-400 bp), a second poly A sequence, a spacer nucleic acid sequence, and the 3′ ITR.

In some embodiments, the first and/or second poly A sequence is a hGH poly A sequence, and in some embodiments, the first and second poly A sequences are a synthetic poly A sequence. In some embodiments, the first poly A sequence is a hGH poly A sequence and the second poly A sequence is a synthetic sequence, or vice versa—that is, in alternative embodiments, the first poly A sequence is a synthetic poly A sequence and the second poly A sequence is a hGH polyA sequence. As a non-limiting example, first poly A is a 49 bp poly A as described in Levitt et al., and second poly A is Reverse poly A or, Reverse RNA Pol II terminator sequence. In some embodiments, only one poly A sequence is used.

In some embodiments, the poly A sequence is selected from any of: SEQ ID NO: 42, SEQ ID NO: 43, SEQ ID NO: 44 or SEQ ID NO: 514, where SEQ ID NO: 44 comprises the signal AATAAA, or a poly A nucleic acid sequence having at least 80%, 85%, 90%, 95%, 96%, 97%, 98% or 99% nucleotide sequence identity to any of SEQ ID NOS: 42-44 or 514.

In some embodiments, the poly A sequence is selected from any of: SEQ ID NO: 46 or SEQ ID NO: 47, or a poly A nucleic acid sequence having at least 80%, 85%, 90%, 95%, 96%, 97%, 98% or 99% nucleotide sequence identity to any of SEQ ID NOS: 46 or 47.

In some embodiments, the poly A sequence is, for example, SEQ ID NO: 15 as disclosed in International WO2021102107 (hGH poly A sequence), or a poly A nucleic acid sequence having at least 80%, 85%, 90%, 95%, 96%, 97%, 98% or 99% nucleotide sequence identity to SEQ ID NO: 15 as disclosed in International Application WO2021102107. In some embodiments, the hGHpoly sequence encompassed for use is described in Anderson et al. J. Biol. Chem 264(14); 8222-8229, 1989 (See, e.g., p. 8223, 2nd column, first paragraph) which is incorporated herein in its entirety by reference.

In one embodiment, the recombinant AAV disclosed herein comprises in its genome a transcriptional terminator signal sequence or a transcriptional pause signal sequence in the reverse orientation between polyA, e.g., first poly A and 3′ITR. In one embodiment, the recombinant AAV disclosed herein comprises in its genome a reverse RNAPolII transcriptional terminator signal sequence or a transcriptional pause signal sequence that is in the 3′-5′ orientation between polyA and 3′ITR.

A “reverse RNA Polymerase II terminator sequence” alternatively called a “dsRNA terminator sequence or termination element”, or, “reverse poly A”, is an element that inhibits transcription of double stranded RNA, e.g., from the 3′ ITR.

In 3′ to 5′ orientation, the termination element does not allow the transcription from 3′ITR and hence double stranded RNA is not transcribed from 3′ITR. Any termination element can be used including e.g., inverted natural polyA sequences from any species or synthetic polyA signals, or, fragments thereof; or other nucleic acid structure terminators known in the art. Exemplary polyA signal and/or, transcription terminators include, but are not limited to polyA signals of BGH, SV40, HGH, Betaglobin, RNA polymerase II transcriptional pause signal from alpha 2 globin gene, transcription termination signal for pol III, fragments thereof and any combination thereof.

A ‘reverse poly A’ is a polyA signal sequence placed in a 3′-5′ orientation downstream of the FVIII transgene and upstream of 3′ITR. Any natural or synthetic poly A in 3′-5′ orientation can be used as reverse poly A. In some embodiments, the reverse poly A is the poly A (pA) as described in International Publication No. WO2019143950 and US Application Publication No. US20200340013, which are incorporated herein by reference in entirety. In several embodiments, the ‘reverse poly A’ and ‘the double stranded RNA termination element’ and ‘reverse RNA Polymerase II terminator sequence’ are used interchangeably.

In some embodiments, the poly A signal is a double stranded RNA termination element and/or, a reverse poly A. In some embodiments, the reverse poly A or, double stranded RNA terminator is located after the homologous or, heterologous poly A signal sequence.

In some embodiments, a transcriptional terminator signal sequence is a reverse RNA polymerase II terminator sequence which is, in a 5′ to 3′ orientation SEQ ID NO: 45, or a rev RNA PolII terminator sequence having at least 80%, 85%, 90%, 95%, 96%, 97%, 98% or 99% nucleotide sequence identity to any of SEQ ID NOS: 45, where SEQ ID NO: 45 orientated in a 5′ to 3′ direction is located between the 3′ of the poly A sequence and 5′ of the right ITR sequence (or 3′ ITR).

In some embodiments, a poly-A tail can be engineered to stabilize the RNA transcript that is transcribed from an rAAV vector genome, including a transcript for a heterologous gene, which in one embodiment is a FVIII, and in alternative embodiments, the poly-A tail can be engineered to include elements that are destabilizing.

In some embodiments of the methods to treat hemophilia A as disclosed herein, a recombinant AAV vector comprises at least one polyA sequence located 3′ of the nucleic acid encoding the FVIII gene and 5′ of the 3′ ITR sequence. In some embodiments, the poly A is a full length poly A (fl-polyA) sequence. In some embodiments, the polyA is a truncated polyA sequence as disclosed in International WO2021102107, which is incorporated herein in its entirety.

In an embodiment, a poly-A tail can be engineered to become a destabilizing element by altering the length of the poly-A tail. In an embodiment, the poly-A tail can be lengthened or shortened.

In some embodiments, there is a 3′ untranslated regions (3′UTRs) located between the heterologous gene encoding the FVIII polypeptide and the poly-A tail. In some embodiments, there is a 3′ UTR located 3′ of the nucleic acid sequence encoding the FVIII polypeptide. In some embodiments, a 3′ untranslated region (3′UTR) comprises the nucleotide sequence set forth in 3′ UTR (SEQ ID NO: 110) or a 3′ UTR (SEQ ID NO: 49) as disclosed herein.

In all aspects of the methods for treating Hemophilia A as disclosed herein, the rAAV genome may also comprise a Stuffer DNA nucleic sequence. An exemplary stuffer DNA sequence is SEQ ID NO: 71 as disclosed in International Application WO2021102107, or a nucleic acid sequence having at least 80%, 85%, 90%, 95%, 96%, 97%, 98% or 99% nucleotide sequence identity thereto. In some embodiments, the stuffer sequence is located 3′ of the poly A tail, for example, and is located 5‘ of the’3 ITR sequence. In some embodiments, the stuffer DNA sequence comprises a synthetic polyadenylation signal in the reverse orientation.

In some embodiments, a stuffer nucleic acid sequence (also referred to as a “spacer” nucleic acid fragment) can be located between the poly A sequence and the 3′ ITR (i.e., a stuffer nucleic acid sequence is located 3′ of the polyA sequence and 5′ of the 3′ ITR). Such a stuffer nucleic acid sequence can be about 30 bp, 50 pb, 75 bp, 100 bp, 150 bp, 200 bp, 250 bp, 300 bp or longer than 300 bp. In some embodiments of the methods and compositions as disclosed herein, a stuffer nucleic acid fragment is between 20-50 bp, 50-100 bp, 100-200 bp, 200-300 bp, 300-500 bp, or any integer between 20-500 bp. Exemplary stuffer (or spacer) nucleic acid sequence can be selected from any of: SEQ ID NO: 16, SEQ ID NO: 71 or SEQ ID NO: 78 as disclosed in International Application WO2021102107, or a nucleic acid sequence at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, 99%, identical to SEQ ID NO: 16 or SEQ ID NO: 71 or SEQ ID NO: 78 as disclosed in International Application WO2021102107.

### AAV ITRs

The rAAV vector or genome as disclosed herein for use in the methods to treat Hemophilia A can comprise AAV ITRs that have desirable characteristics and can be designed to modulate the activities of, and cellular responses to vectors that incorporate the ITRs. In another embodiment, the AAV ITRs are synthetic AAV ITRs that has desirable characteristics and can be designed to manipulate the activities of and cellular responses to vectors comprising one or two synthetic ITRs, including, as set forth in U.S. Pat. No. 9,447,433, which is incorporated herein by reference.

In another embodiment, an ITR exhibits modified transcription activity relative to a naturally occurring ITR, e.g., ITR2 from AAV2. It is known that the ITR2 sequence inherently has promoter activity. It also inherently has termination activity, similar to a poly(A) sequence. The minimal functional ITR of the present invention exhibits transcription activity as shown in the examples, although at a diminished level relative to ITR2. Thus, in some embodiments, the ITR is functional for transcription. In other embodiments, the ITR is defective for transcription. In certain embodiments, the ITR can act as a transcription insulator, e.g., preventing transcription of a transgenic cassette present in the vector when the vector is integrated into a host chromosome.

One aspect of the invention relates to an rAAV vector genome comprising at least one synthetic AAV ITR, wherein the nucleotide sequence of one or more transcription factor binding sites in the ITR is deleted and/or substituted, relative to the sequence of a naturally occurring AAV ITR such as ITR2. In some embodiments, it is the minimal functional ITR in which one or more transcription factor binding sites are deleted and/or substituted. In some embodiments at least 1 transcription factor binding site is deleted and/or substituted, e.g., at least 5 or more or 10 or more transcription factor binding sites, e.g., at least 1, 2, 3, 4, 5, 6, 7, 8, 9, 10, 11, 12, 13, 14, 15, 16, 17, 18, 19, 20, or 21 transcription factor binding sites.

Another embodiment, a rAAV vector, including an rAAV vector genome as described herein comprises a polynucleotide comprising at least one synthetic AAV ITR, wherein one or more CpG islands (a cytosine base followed immediately by a guanine base (a CpG) in which the cytosines in such arrangement tend to be methylated) that typically occur at, or near the transcription start site in an ITR are deleted and/or substituted. In an embodiment, deletion, or reduction in the number of CpG islands can reduce the immunogenicity of the rAAV vector. This results from a reduction or complete inhibition in TLR-9 binding to the rAAV vector DNA sequence, which occurs at CpG islands. It is also well known that methylation of CpG motifs results in transcriptional silencing. Removal of CpG motifs in the ITR is expected to result in decreased TLR-9 recognition and/or decreased methylation and therefore decreased transgene silencing. In some embodiments, it is the minimal functional ITR in which one or more CpG islands are deleted and/or substituted. In an embodiment, AAV ITR2 is known to contain 16 CpG islands of which one or more, or all 16 can be deleted.

In some embodiments, at least 1 CpG motif is deleted and/or substituted, e.g., at least 4 or more or 8 or more CpG motifs, e.g., at least 1, 2, 3, 4, 5, 6, 7, 8, 9, 10, 11, 12, 13, 14, 15, or 16 CpG motifs.

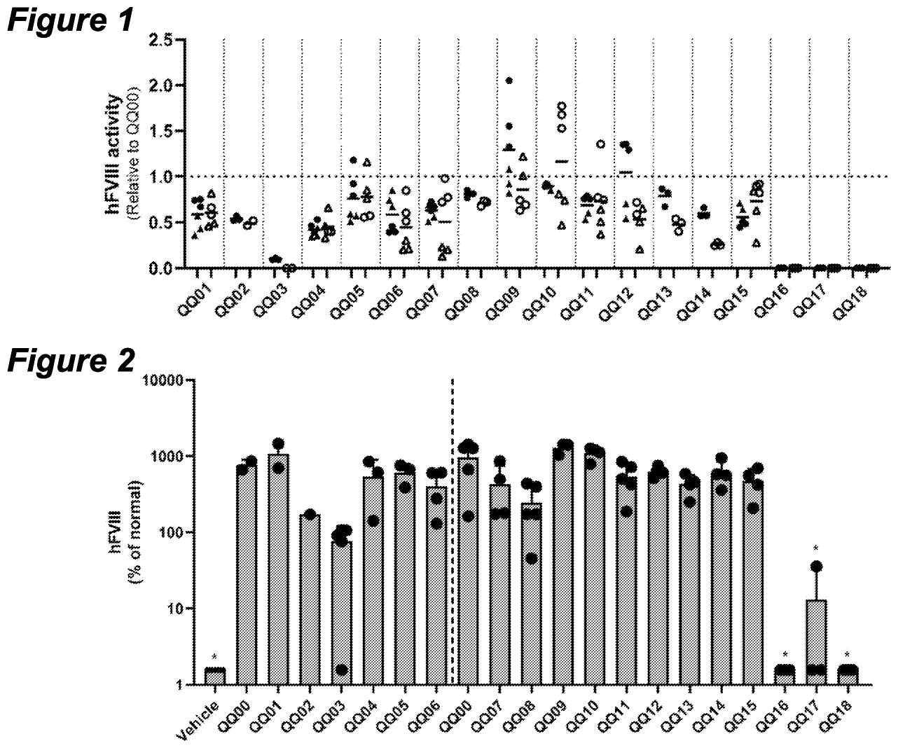

In another embodiment, the synthetic ITR comprises, consists essentially of, or consists of one of the nucleotide sequences listed in Table 1. In other embodiments, the synthetic ITR comprises, consist essentially of, or consist of a nucleotide sequence that is at least 80% identical, e.g., at least 85%, 90%, 95%, 96%, 97%, 98%, or 99% identical to any one of the nucleotide sequences listed in Table 1. In some embodiments, the ITR is a sequence is disclosed in FIG. 1 of Samulski et al., 1983, Cell, 33; 135-143 (referred to “Samulski et al, 1983” as which is incorporated herein in its entirety by reference), which discloses modified ITR sequences in FIG. 1. In some embodiments, the ITR sequence comprises, or consists of a nucleotide sequence that is at least 80% identical, e.g., at least 85%, 90%, 95%, 96%, 97%, 98%, or 99% identical to one of the ITR sequences in FIG. 1 as disclosed in Samulski et al, 1993. In some embodiments, the ITR comprises, or consists of a nucleotide sequence that is at least 80% identical, e.g., at least 85%, 90%, 95%, 96%, 97%, 98%, or 99% or 99.5% identical to the ITR sequence of pSM 609 right disclosed in the middle panel of FIG. 1 (that lacks the 9 bp) disclosed in Samulski et al, 1983. In some embodiments, the ITR comprises a nucleotide sequence that is at least 80% identical, e.g., at least 85%, 90%, 95%, 96%, 97%, 98%, or 99% or 99.5% identical to the ITR sequence of any of SEQ ID NOs: 79-84 and 450-451.

In some embodiments, the ITR sequence, e.g., Right ITR (or 3′ ITR) is SEQ ID NO: 80 or SEQ ID NO: 82 or a nucleotide sequence that is at least 80% identical, e.g., at least 85%, 90%, 95%, 96%, 97%, 98%, or 99% or 99.5% identical to SEQ ID NO: 80 or SEQ ID NO: 82. In some embodiments, the ITR sequence, e.g., left ITR (or 5′ ITR) is SEQ ID NO: 79 or SEQ ID NO: 81 or a nucleotide sequence that is at least 80% identical, e.g., at least 85%, 90%, 95%, 96%, 97%, 98%, or 99% or 99.5% identical to SEQ ID NO: 79 or SEQ ID NO: 81.

In some embodiments of any aspect of the invention, one or both of the ITR sequences is a wt ITR or an ITR sequence disclosed herein, having an insertion, deletion or substitution. In one embodiment of any aspect of this invention, any one or, both of 5′ITR and 3′ITR is 145 bp long or, smaller than 145 bp in length e.g, 142 bp, 141 bp, 140 bp, 135 bp, 130 bp, 128 bp, 120 bp, 117 bp, 115 bp, or, smaller than 115 bp in length. In one embodiment, the 5′ITR or, 3′ITR is 130 bp long. In one embodiment, both 5′ITR and 3′ITR are 130 bp long.

TABLE 1
Exemplary synthetic ITR sequences

| | |
|---|---|
| L-ITR | CTGCGCGCTCGCTCGCTCACTGAGGCCGCCCGGGCAAAGCCCGGGCGTC |
| | GGGCGACCTTTGGTCGCCCGGCCTCAGTGAGCGAGCGAGCGCGCAGAGA |
| | GGGAGTGGCCAACTCCATCACTAGGGGTTCCT (SEQ ID NO: 79) |
| R-ITR | AGGAACCCCTAGTGATGGAGTTGGCCACTCCCTCTCTGCGCGCTCGCTC |
| | GCTCACTGAGGCCGGGCGACCAAAGGTCGCCCGACGCCCGGGCTTTGCC |
| | CGGGCGGCCTCAGTGAGCGAGCGAGCGCGCAG (SEQ ID NO: 80) |
| L-ITR | CTGCGCGCTCGCTCGCTCACTGAGGCCGCCCGGGCAAAGCCCGGGCGTC |
| | GGGCGACCTTTGGTCGCCCGGCCTCAGTGAGCGAGCGAGCGCGCAGAGA |
| | GGGAGTGGCCAACTCCATCACTAGGG (SEQ ID NO: 81) |
| R-ITR | AGGAACCCCTAGTGATGGAGTTGGCCACTCCCTCTCTGCGCGCTCGCTC |
| | GCTCACTGAGGCCGGGCGACCAAAGGTCGCCCGACGCCCGGGCTTTGCC |
| | CGGGCGGCCTCAGTGAGCGAGCGAGCGCGCAG (SEQ ID NO: 82) |
| MH-257 | AGGAACCCCTAGTGATGGAGTTGGCCACTCCCTCTCTGCGCGCTCGCTC |
| | GCTCACTGAGGCAATTTGATAAAAATCGTCAAATTATAAACAGGCTTTG |
| | CCTGTTTAGCCTCAGTGAGCGAGCGAGCGCGCAGAGAGGGAGTGGCCAA |
| | CTCCATCACTAGGGGTTCCT (SEQ ID NO: 83) |
| MH-258 | AGGAACCCCTAGTGATGGAGTTGGCCACTCCCTCTCTGCGCGCTCGCTC |
| | GCTCACTGAGGGATAAAAATCCAGGCTTTGCCTGCCTCAGTGAGCGAGC |
| | GAGCGCGCAGAGAGGGAGTGGCCAACTCCATCACTAGGGGTTCCT |
| | (SEQ ID NO: 84) |
| MH Delta | AGGAACCCCTAGTGATGGAGTTGGCCACTCCCTCTCTGCGCGCTCGCTC |
| 258 | GCTCACTGAGGGATAAAAATCCAGGCTTTGCCTGCCTCAGTGAGCGAGC |
| | GAGCGCGCAGAGAGGGAGTGGCCAACTCCATCACTAGGGGTTCCT |
| | (SEQ ID NO: 85) |
| MH Telomere- | AGGAACCCCTAGTGATGGAGTTGGCCACTCCCTCTCTGGGATTGGGATT |
| 1 ITR | GCGCGCTCGCTCGCGGGATTGGGATTGGGATTGGGATTGGGATTGGGAT |
| | TGATAAAAATCAATCCCAATCCCAATCCCAATCCCAATCCCAATCCCGC |
| | GAGCGAGCGCGCAATCCCAATCCCAGAGAGGGAGTGGCCAACTCCATCA |
| | CTAGGGGTTCCT (SEQ ID NO: 86) |
| MH Telomere- | AGGAACCCCTAGTGATGGAGTTGGCCACTCCCTCTCTGCGCGCTCGCTC |
| 2 ITR | GCTCGGGATTGGGATTGGGATTGGGATTGGGATTGGGATTGATAAAAAT |
| | CAATCCCAATCCCAATCCCAATCCCAATCCCAATCCCGCGAGCGAGCGC |
| | GCAGGAGAGGGAGTGGCCAACTCCATCACTAGGGGTTCCTAAGCTTATT |
| | ATA (SEQ ID NO: 87) |
| MH PolII 258 | AGGAACCCCTAGTGATGGAGTTGGCCACTCCCTCTCTGCGCGCTCGCTC |
| ITR | GCTCACTGAGGGCGCCTATAAAGATAAAAATCCAGGCTTTGCCTGCCTC |
| | AGTTAGCGAGCGAGCGCGCAGAGAGGGAGTGGCCAACTCCATCACTAGG |
| | GGTTCCT (SEQ ID NO: 88) |
| MH 258 | CTAGTGATGGAGTTGGCCACTCCCTCTCTGCGCGCTCGCTCGCTCACTG |
| Delta D | AGGGATAAAAATCCAGGCTTTGCCTGCCTCAGTGAGCGAGCGAGCGCGC |
| conservative | AGAGAGGGAGTGGCCAACTCCATCACTAG (SEQ ID NO: 89) |
| 5′ ITR | TTGGCCACTCCCTCTCTGCGCGCTCGCTCGCTCACTGAGGCCGGGCGAC |
| | CAAAGGTCGCCCGACGCCCGGGCTTTGCCCGGGCGGCCTCAGTGAGCGA |
| | GCGAGCGCGCAGAGAGGGAGTGGCCAACTCCATCACTAGGGGTTCCT |
| | (SEQ ID NO: 104) |
| 3′ ITR | AGGAACCCCTAGTGATGGAGTTGGCCACTCCCTCTCTGCGCGCTCGCTC |
| | GCTCACTGAGGCCGGGCGACCAAAGGTCGCCCGACGCCCGGGCTTTGCC |
| | CGGGCGGCCTCAGTGAGCGAGCGAGCGCGCAGAGAGGGAGTGGCCAA |
| | (SEQ ID NO: 105) |
| ITR (145bp) | AGGAACCCCTAGTGATGGAGTTGGCCACTCCCTCTCTGCGCGCTCGCTC |
| | GCTCACTGAGGCCGGGCGACCAAAGGTCGCCCGACGCCCGGGCTTTGCC |
| | CGGGCGGCCTCAGTGAGCGAGCGAGCGCGCAGAGAGGGAGTGGCCAA |
| | (SEQ ID NO: 448) |
| ITR | AGGAACCCCTAGTGATGGAGTTGGCCACTCCCTCTCTGCGCGCTCGCTC |
| (145bp-1983, | GCTCACTGAGGCCGGGCGACCAAAGGTCGCCCGACGCCCGGGCTTTGCC |
| lacking 9bp) | CGGGCGGCCTCAGTGAGCGAGCGAGCGCGCAGAGAGGG (SEQ ID |
| | NO: 449) |

### Vectors and Virions

In one embodiment, the rAAV vector (also referred to as a rAAV virion) as disclosed herein comprises a capsid protein, and a rAAV genome in the capsid protein. A rAAV capsid of the rAAV virion used to treat Hemophilia A is any of those listed in Table 2 herein, or in Table 1 as disclosed in International Applications WO2020/102645, and WO2020/102667, each of which are incorporated herein in their entirety. In one embodiment, a rAAV capsid of the rAAV virion used to treat Hemophilia A is an AAV8 capsid. In one embodiment, a rAAV vector is an rAAV8 vector. As a further embodiment, the rAAV of the invention described herein comprises AAV capsid proteins that can be polyploid (also referred to as haploid, or rational haploid, or rational polyploid), i.e., they can comprise VP1, VP2, and VP3 capsid proteins from more than one AAV serotypes in a single AAV virion as described in International Application Nos PCT/US2018/022725, PCT/US2018/044632, and U.S. Pat. No. 10,550,405; all of which are incorporated here by reference. In some embodiments, rAAV comprises at least one capsid protein of VP1, VP2, and VP3 selected from AAV serotypes listed in Table 2.

TABLE 2
Table 2: AAV Serotypes and exemplary Published corresponding capsid sequence

| | Serotype and where capsid sequence is |
|---|---|
| Serotype and where capsid sequence is published | published |
| AAV3.3b (See SEQ ID NO: 72 in US20030138772) | AAV3-3 (See SEQ ID NO: 200 |
| | US20150315612) |
| AAV3-3 (See SEQ ID NO: 217 US20150315612) | AAV3a ((See SEQ ID NO: 5 in U.S. Pat. No. 6,156,303) |
| AAV3a (See SEQ ID NO: 9 in U.S. Pat. No. 6,156,303) | AAV3b (See SEQ ID NO: 6 in U.S. Pat. No. 6,156,303) |
| AAV3b (See SEQ ID NO: 10 in U.S. Pat. No. 6,156,303) | AAV3b (See SEQ ID NO: 1 in U.S. Pat. No. 6,156,303) |
| AAV4 (See SEQ ID NO: 17 US20140348794) | AAV4 ((See SEQ ID NO: 5 in US20140348794) |
| AAV4 (See SEQ ID NO: 3 in US20140348794) | AAV4 (See SEQ ID NO: 14 in |
| | US20140348794) |
| AAV4 (See SEQ ID NO: 15 in US20140348794) | AAV4 (See SEQ ID NO: 19 in |
| | US20140348794) |
| AAV4 (See SEQ ID NO: 12 in US20140348794) | AAV4 (See SEQ ID NO: 13 in |
| | US20140348794) |
| AAV4 (See SEQ ID NO: 7 in US20140348794) | AAV4 (See SEQ ID NO: 8 in |
| | US20140348794) |
| AAV4 (See SEQ ID NO: 9 in US20140348794) | AAV4 (See SEQ ID NO: 2 in |
| | US20140348794) |
| AAV4 (See SEQ ID NO: 10 in US20140348794) | AAV4 (See SEQ ID NO: 11 in |
| | US20140348794) |
| AAV4 (See SEQ ID NO: 18 in US20140348794) | AAV4 (See SEQ ID NO: 63 in |
| | US20030138772) and US20160017295 SEQ |
| ID NO: (See SEQ ID NO: 4 in US20140348794) | AAV4 (See SEQ ID NO: 16 in |
| | US20140348794) |
| AAV4 (See SEQ ID NO: 20 in US20140348794) | AAV4 (See SEQ ID NO: 6 in |
| | US20140348794) |
| AAV4 (See SEQ ID NO: 1 in US20140348794) | AAV42.2 (See SEQ ID NO: 9 in |
| | US20030138772) |
| AAV42.2 (See SEQ ID NO: 102 in | AAV42.3b (See SEQ ID NO: 36 in |
| US20030138772) | US20030138772) |
| AAV42.3B (See SEQ ID NO: 107 in | AAV42.4 (See SEQ ID NO: 33 in |
| US20030138772) | US20030138772) |
| AAV42.4 (See SEQ ID NO: 88 in | AAV42.8 (See SEQ ID NO: 27 in |
| US20030138772) | US20030138772) |
| AAV42.8 (See SEQ ID NO: 85 in | AAV43.1 (See SEQ ID NO: 39 in |
| US20030138772) | US20030138772) |
| AAV43.1 (See SEQ ID NO: 92 in | AAV43.12 (See SEQ ID NO: 41 in |
| US20030138772) | US20030138772) |
| AAV43.12 (See SEQ ID NO: 93 in | AAV8 (See SEQ ID NO: 15 in |
| US20030138772) | US20150159173) |
| AAV8 (See SEQ ID NO: 7 in US20150376240) | AAV8 (See SEQ ID NO: 4 in |
| | US20030138772; US20150315612 SEQ |
| ID NO: 182 | AAV8 (See SEQ ID NO: 95 in |
| | US20030138772), US20140359799 SEQ |
| AAV8 (See SEQ ID NO: 31 in US20150159173) | AAV8 (See, e.g., SEQ ID NO: 8 in |
| | US20160017295, or SEQ ID NO: 7 in |
| | U.S. Pat. No. 7,198,951, or SEQ ID NO: 223 in |
| | US20150315612) |
| AAV8 (See SEQ ID NO: 8 in US20150376240) | AAV8 (See SEQ ID NO: 214 in |
| | US20150315612) |
| AAV-8b (See SEQ ID NO: 5 in US20150376240) | AAV-8b (See SEQ ID NO: 3 in |
| | US20150376240) |
| AAV-8h (See SEQ ID NO: 6 in US20150376240) | AAV-8h (See SEQ ID NO: 4 in |
| | US20150376240) |
| AAV9 (See SEQ ID NO: 5 in US20030138772) | AAV9 (See SEQ ID NO: 1 in U.S. Pat. No. 7,198,951) |
| AAV9 (See SEQ ID NO: 9 in US20160017295) | AAV9 (See SEQ ID NO: 100 in |
| | US20030138772), U.S. Pat. No. 7,198,951 SEQ ID NO: 2 |
| | AAV9 (See SEQ ID NO: 3 in U.S. Pat. No. 7,198,951) |
| AAV9 (AAVhu.14) (See SEQ ID NO: 3 in | AAV9 (AAVhu.14) (See SEQ ID NO: 123 in |
| US20150315612) | US20150315612) |
| AAVA3.1 (See SEQ ID NO: 120 in | AAVA3.3 (See SEQ ID NO: 57 in |
| US20030138772) | US20030138772) |
| AAVA3.3 (See SEQ ID NO: 66 in | AAVA3.4 (See SEQ ID NO: 54 in |
| US20030138772) | US20030138772) |
| AAVA3.4 (See SEQ ID NO: 68 in | AAVA3.5 (See SEQ ID NO: 55 in |
| US20030138772) | US20030138772) |
| AAVA3.5 (See SEQ ID NO: 69 in | AAVA3.7 (See SEQ ID NO: 56 in |
| US20030138772) | US20030138772) |
| AAVA3.7 (See SEQ ID NO: 67 in | AAV29. (See SEQ ID NO: 11 in (AAVbb.l) |
| US20030138772) | 161 US20030138772) |
| AAVC2 (See SEQ ID NO: 61 in US20030138772) | AAVCh.5 (See SEQ ID NO: 46 in |
| | US20150159173); US20150315612 SEQ |
| ID NO: 234 | AAVcy.2 (AAV13.3) (See SEQ ID NO: 15 in |
| | US20030138772) |
| AAV24.1 (See SEQ ID NO: 101 in | AAVcy.3 (AAV24.1) (See SEQ ID NO: 16 in |
| US20030138772) | US20030138772) |
| AAV27.3 (See SEQ ID NO: 104 in | AAVcy.4 (AAV27.3) (See SEQ ID NO: 17 in |
| US20030138772) | US20030138772) |
| AAVcy.5 (See SEQ ID NO: 227 in | AAV7.2 (See SEQ ID NO: 103 in |
| US20150315612) | US20030138772) |
| AAVcy.5 (AAV7.2) (See SEQ ID NO: 18 in | AAV16.3 (See SEQ ID NO: 105 in |
| US20030138772) | US20030138772) |
| AAVcy.6 (AAV16.3) (See SEQ ID NO: 10 in | AAVcy.5 (See SEQ ID NO: 8 in |
| US20030138772) | US20150159173) |
| AAVcy.5 (See SEQ ID NO: 24 in | AAVCy.5Rl (See SEQ ID NO: in |
| US20150159173) | US20150159173 |
| AAVCy.5R2 (See SEQ ID NO: in | AAVCy.5R3 (See SEQ ID NO: in |
| US20150159173) | US20150159173 |
| AAVCy.5R4 (See SEQ ID NO: in | AAVDJ (See SEQ ID NO: 3 in |
| US20150159173) | US20140359799) and SEQ ID NO: 2 in |
| | U.S. Pat. No. 7,588,772) |
| | AAVDJ (See SEQ ID NO: 2 in |
| | US20140359799; and SEQ ID NO: 1 in |
| | U.S. Pat. No. 7,588,772) |
| | AAVDJ-8 (See SEQ ID NO: in U.S. Pat. No. 7,588,772; |
| | Grimm et al 2008 |
| AAVDJ-8 (See SEQ ID NO: in U.S. Pat. No. 7,588,772; | AAVF5 (See SEQ ID NO: 110 in |
| Grimm et al 2008 | US20030138772) |
| AAVH2 (See SEQ ID NO: 26 in US20030138772) | AAVH6 (See SEQ ID NO: 25 in |
| | US20030138772) |
| AAVhEl.1 (See SEQ ID NO: 44 in U.S. Pat. No. 9,233,131) | AAVhErl.14 (See SEQ ID NO: 46 in |
| | U.S. Pat. No. 9,233,131) |
| AAVhErl.16 (See SEQ ID NO: 48 in U.S. Pat. No. 9,233,131) | AAVhErl.18 (See SEQ ID NO: 49 in |
| | U.S. Pat. No. 9,233,131) |
| AAVhErl.23 (AAVhEr2.29) (See SEQ ID NO: 53 | AAVhErl.35 (See SEQ ID NO: 50 in |
| in U.S. Pat. No. 9,233,131) | U.S. Pat. No. 9,233,131) |
| AAVhErl.36 (See SEQ ID NO: 52 in U.S. Pat. No. 9,233,131) | AAVhErl.5 (See SEQ ID NO: 45 in |
| | U.S. Pat. No. 9,233,131) |
| AAVhErl.7 (See SEQ ID NO: 51 in U.S. Pat. No. 9,233,131) | AAVhErl.8 (See SEQ ID NO: 47 in |
| | U.S. Pat. No. 9,233,131) |
| AAVhEr2.16 (See SEQ ID NO: 55 in U.S. Pat. No. 9,233,131) | AAVhEr2.30 (See SEQ ID NO: 56 in |
| | U.S. Pat. No. 9,233,131) |
| AAVhEr2.31 (See SEQ ID NO: 58 in U.S. Pat. No. 9,233,131) | AAVhEr2.36 (See SEQ ID NO: 57 in |
| | U.S. Pat. No. 9,233,131) |
| AAVhEr2.4 (See SEQ ID NO: 54 in U.S. Pat. No. 9,233,131) | AAVhEr3.1 (See SEQ ID NO: 59 in |
| | U.S. Pat. No. 9,233,131) |
| AAVhu.l (See SEQ ID NO: 46 in US20150315612) | AAVhu.l (See SEQ ID NO: 144 in |
| | US20150315612) |
| AAVhu.lO (AAV16.8) (See SEQ ID NO: 56 in | AAVhu.lO (AAV16.8) (See SEQ ID NO: 156 |
| US20150315612) | in US20150315612) |
| AAVhu.ll (AAV16.12) (See SEQ ID NO: 57 in | AAVhu.ll (AAV16.12) (See SEQ ID NO: 153 |
| US20150315612) | in US20150315612) |
| AAVhu.12 (See SEQ ID NO: 59 in | AAVhu.12 (See SEQ ID NO: 154 in |
| US20150315612) | US20150315612) |
| AAVhu.13 (See SEQ ID NO: 16 in | |
| US2015015917 and ID NO: 71 in US20150315612) | |
| AAVhu.13 (See SEQ ID NO: 32 in | |
| US20150159173 and ID NO: 129 US20150315612) | |
| AAVhu.136.1 (See SEQ ID NO: 165 in | AAVhu.140.1 (See SEQ ID NO: 166 in |
| US20150315612) | US20150315612) |
| AAVhu.140.2 (See SEQ ID NO: 167 in | AAVhu.145.6 (See SEQ ID NO: 178 in |
| US20150315612) | US20150315612) |
| AAVhu.15 (See SEQ ID NO: 147 in | AAVhu.15 (AAV33.4) (See SEQ ID NO: 50 in |
| US20150315612) | US20150315612) |
| AAVhu.156.1 (See SEQ ID NO: 179 in | AAVhu.16 (See SEQ ID NO: 148 in |
| US20150315612) | US20150315612) |
| AAVhu.l6 (AAV33.8) (See SEQ ID NO: 51 in | AAVhu.17 (See SEQ ID NO: 83 in |
| US20150315612) | US20150315612) |
| AAVhu.l7 (AAV33.12) (See SEQ ID NO: 4 in | AAVhu.172.1 (See SEQ ID NO: 171 in |
| US20150315612) | US20150315612) |
| AAVhu.172.2 (See SEQ ID NO: 172 in | AAVhu.173.4 (See SEQ ID NO: 173 in |
| US20150315612) | US20150315612) |
| AAVhu.173.8 (See SEQ ID NO: 175 in | AAVhu.18 (See SEQ ID NO: 52 in |
| US20150315612) | US20150315612) |
| AAVhu.18 (See SEQ ID NO: 149 in | AAVhu.19 (See SEQ ID NO: 62 in |
| US20150315612) | US20150315612) |
| AAVhu.19 (See SEQ ID NO: 133 in | AAVhu.2 (See SEQ ID NO: 48 in |
| US20150315612) | US20150315612) |
| AAVhu.2 (See SEQ ID NO: 143 in | AAVhu.20 (See SEQ ID NO: 63 in |
| US20150315612) | US20150315612) |
| AAVhu.20 (See SEQ ID NO: 134 in | AAVhu.21 (See SEQ ID NO: 65 in |
| US20150315612) | US20150315612) |
| AAVhu.21 (See SEQ ID NO: 135 in | AAVhu.22 (See SEQ ID NO: 67 in |
| US20150315612) | US20150315612) |
| AAVhu.22 239 (See SEQ ID NO: 138 in | AAVhu.23 (See SEQ ID NO: 60 in |
| US20150315612) | US20150315612) |
| AAVhu.23.2 (See SEQ ID NO: 137 in | AAVhu.24 (See SEQ ID NO: 66 in |
| US20150315612) | US20150315612) |
| AAVhu.24 (See SEQ ID NO: 136 in | AAVhu.25 (See SEQ ID NO: 49 in |
| US20150315612) | US20150315612) |
| AAVhu.25 (See SEQ ID NO: 146 in | AAVhu.26 (See SEQ ID NO: 17 in |
| US20150315612) | US20150159173 and SEQ ID NO: 61 in |
| | US20150315612) |
| | AAVhu.26 (See SEQ ID NO: 33 in |
| | US20150159173), US20150315612 SEQ |
| | AAVhu.27 (See SEQ ID NO: 64 in |
| | US20150315612) |
| AAVhu.27 (See SEQ ID NO: 140 in | AAVhu.28 (See SEQ ID NO: 68 in |
| US20150315612) | US20150315612) |
| AAVhu.28 (See SEQ ID NO: 130 in | AAVhu.29 (See SEQ ID NO: 69 in |
| US20150315612) | US20150315612) |
| AAVhu.29 (See SEQ ID NO: 42 in | |
| US20150159173 and SEQ ID NO: 132 in | |
| US20150315612) | |
| AAVhu.29 (See SEQ ID NO: 225 in | AAVhu.29R (See SEQ ID NO: in |
| US20150315612) | US20150159173 |
| AAVhu.3 (See SEQ ID NO: 44 in | AAVhu.3 (See SEQ ID NO: 145 in |
| US20150315612) | US20150315612) |
| AAVhu.30 (See SEQ ID NO: 70 in | AAVhu.30 (See SEQ ID NO: 131 in |
| US20150315612) | US20150315612) |
| AAVhu.31 (See SEQ ID NO: 1 in | AAVhu.31 (See SEQ ID NO: 121 in |
| US20150315612) | US20150315612) |
| AAVhu.32 (See SEQ ID NO: 2 in | AAVhu.32 (See SEQ ID NO: 122 in |
| US20150315612) | US20150315612) |
| AAVhu.33 (See SEQ ID NO: 75 in | AAVhu.33 (See SEQ ID NO: 124 in |
| US20150315612) | US20150315612) |
| AAVhu.34 (See SEQ ID NO: 72 in | AAVhu.34 (See SEQ ID NO: 125 in |
| US20150315612) | US20150315612) |
| AAVhu.35 (See SEQ ID NO: 73 in | AAVhu.35 (See SEQ ID NO: 164 in |
| US20150315612) | US20150315612) |
| AAVhu.36 (See SEQ ID NO: 74 in | AAVhu.36 (See SEQ ID NO: 126 in |
| US20150315612) | US20150315612) |
| AAVhu.37 (See SEQ ID NO: 34 in | |
| US20150159173 and SEQ ID NO: 88 in | |
| US20150315612) | |
| AAVhu.37 (AAV106.1) (See SEQ ID NO: 10 in | |
| US20150315612 and SEQ ID NO: 18 in | |
| US20150159173) | |
| AAVhu.38 (See SEQ ID NO: 161 in | AAVhu.39 (See SEQ ID NO: 102 in |
| US20150315612) | US20150315612) |
| AAVhu.39 (AAVLG-9) (See SEQ ID NO: 24 in | AAVhu.4 (See SEQ ID NO: 47 in |
| US20150315612) | US20150315612) |
| AAVhu.4 (See SEQ ID NO: 141 in | AAVhu.40 (See SEQ ID NO: 87 in |
| US20150315612) | US20150315612) |
| AAVhu.40 (AAV114.3) (See SEQ ID NO: 11 in | AAVhu.41 (See SEQ ID NO: 91 in |
| US20150315612) | US20150315612) |
| AAVhu.41 (AAV127.2) (See SEQ ID NO: 6 in | AAVhu.42 (See SEQ ID NO: 85 in |
| US20150315612) | US20150315612) |
| AAVhu.42 (AAV127.5) (See SEQ ID NO: 8 in | AAVhu.43 (See SEQ ID NO: 160 in |
| US20150315612) | US20150315612) |
| AAVhu.43 (See SEQ ID NO: 236 in | AAVhu.43 (AAV128.1) (See SEQ ID NO: 80 |
| US20150315612) | in US20150315612) |
| AAVhu.44 (See SEQ ID NO: 45 in | |
| US20150159173 and SEQ ID NO: 158 in | |
| US20150315612) | |
| AAVhu.44 (AAV128.3) (See SEQ ID NO: 81 in | AAVhu.44Rl (See SEQ ID NO: in |
| US20150315612) | US20150159173 |
| AAVhu.44R2 (See SEQ ID NO: in | AAVhu.44R3 (See SEQ ID NO: in |
| US20150159173 | US20150159173 |
| AAVhu.45 (See SEQ ID NO: 76 in | AAVhu.45 (See SEQ ID NO: 127 in |
| US20150315612) | US20150315612) |
| AAVhu.46 (See SEQ ID NO: 82 in | AAVhu.46 (See SEQ ID NO: 159 in |
| US20150315612) | US20150315612) |
| AAVhu.46 (See SEQ ID NO: 224 in | AAVhu.47 (See SEQ ID NO: 77 in |
| US20150315612) | US20150315612) |
| AAVhu.47 (See SEQ ID NO: 128 in | AAVhu.48 (See SEQ ID NO: 38 in |
| US20150315612) | US20150159173) |
| AAVhu.48 (See SEQ ID NO: 157 in | AAVhu.48 (AAV130.4) (See SEQ ID NO: 78 |
| US20150315612) | in US20150315612) |
| AAVhu.48Rl (See SEQ ID NO: in | AAVhu.48R2 (See SEQ ID NO: in |
| US20150159173 | US20150159173 |
| AAVhu.48R3 (See SEQ ID NO: in | AAVhu.49 (See SEQ ID NO: 209 in |
| US20150159173 | US20150315612) |
| AAVhu.49 (See SEQ ID NO: 189 in | AAVhu.5 (See SEQ ID NO: 45 in |
| US20150315612) | US20150315612) |
| AAVhu.5 (See SEQ ID NO: 142 in | AAVhu.51 (See SEQ ID NO: 208 in |
| US20150315612) | US20150315612) |
| AAVhu.51 (See SEQ ID NO: 190 in | AAVhu.52 (See SEQ ID NO: 210 in |
| US20150315612) | US20150315612) |
| AAVhu.52 (See SEQ ID NO: 191 in | AAVhu.53 (See SEQ ID NO: 19 in |
| US20150315612) | US20150159173) |
| AAVhu.53 (See SEQ ID NO: 35 in | AAVhu.53 (AAV145.1) (See SEQ ID NO: 176 |
| US20150159173) | in US20150315612) |
| AAVhu.54 (See SEQ ID NO: 188 in | AAVhu.54 (AAV145.5) (See SEQ ID NO: 177 |
| US20150315612) | in US20150315612) |
| AAVhu.55 (See SEQ ID NO: 187 in | AAVhu.56 (See SEQ ID NO: 205 in |
| US20150315612) | US20150315612) |
| AAVhu.56 (AAV145.6) (See SEQ ID NO: 168 in | AAVhu.56 (AAV145.6) (See SEQ ID NO: 192 |
| US20150315612) | in US20150315612) |
| AAVhu.57 (See SEQ ID NO: 206 in | AAVhu.57 (See SEQ ID NO: 169 in |
| US20150315612) | US20150315612) |
| AAVhu.57 (See SEQ ID NO: 193 in | AAVhu.58 (See SEQ ID NO: 207 in |
| US20150315612) | US20150315612) |
| AAVhu.58 (See SEQ ID NO: 194 in | AAVhu.6 (AAV3.1) (See SEQ ID NO: 5 in |
| US20150315612) | US20150315612) |
| AAVhu.6 (AAV3.1) (See SEQ ID NO: 84 in | AAVhu.60 (See SEQ ID NO: 184 in |
| US20150315612) | US20150315612) |
| AAVhu.60 (AAV161.10) (See SEQ ID NO: 170 in | AAVhu.61 (See SEQ ID NO: 185 in |
| US20150315612) | US20150315612) |
| AAVhu.61 (AAV161.6) (See SEQ ID NO: 174 in | AAVhu.63 (See SEQ ID NO: 204 in |
| US20150315612) | US20150315612) |
| AAVhu.63 (See SEQ ID NO: 195 in | AAVhu.64 (See SEQ ID NO: 212 in |
| US20150315612) | US20150315612) |
| AAVhu.64 (See SEQ ID NO: 196 in | AAVhu.66 (See SEQ ID NO: 197 in |
| US20150315612) | US20150315612) |
| AAVhu.67 (See SEQ ID NO: 215 in | AAVhu.67 (See SEQ ID NO: 198 in |
| US20150315612) | US20150315612) |
| AAVhu.7 (See SEQ ID NO: 226 in | AAVhu.7 (See SEQ ID NO: 150 in |
| US20150315612) | US20150315612) |
| AAVhu.7 (AAV7.3) (See SEQ ID NO: 55 in | AAVhu.71 (See SEQ ID NO: 79 in |
| US20150315612) | US20150315612) |
| AAVhu.8 (See SEQ ID NO: 53 in | AAVhu.8 (See SEQ ID NO: 12 in |
| US20150315612) | US20150315612) |
| AAVhu.8 (See SEQ ID NO: 151 in | AAVhu.9 (AAV3.1) (See SEQ ID NO: 58 in |
| US20150315612) | US20150315612) |
| AAVhu.9 (AAV3.1) (See SEQ ID NO: 155 in | AAV-LK01 (See SEQ ID NO: 2 in |
| US20150315612) | US20150376607) |
| AAV-LK01 (See SEQ ID NO: 29 in | AAV-LK02 (See SEQ ID NO: 3 in |
| US20150376607) | US20150376607) |
| AAV-LK02 (See SEQ ID NO: 30 in | AAV-LK03 (See SEQ ID NO: 4 in |
| US20150376607) | US20150376607) |
| AAV-LK03 (See SEQ ID NO: 12 in | |
| WO2015121501 and SEQ ID NO: 31 in | |
| US20150376607) | |
| AAV-LK04 (See SEQ ID NO: 5 in | AAV-LK04 (See SEQ ID NO: 32 in |
| US20150376607) | US20150376607) |
| AAV-LK05 (See SEQ ID NO: 6 in | AAV-LK05 (See SEQ ID NO: 33 in |
| US20150376607) | US20150376607) |
| AAV-LK06 (See SEQ ID NO: 7 in | AAV-LK06 (See SEQ ID NO: 34 in |
| US20150376607) | US20150376607) |
| AAV-LK07 (See SEQ ID NO: 8 in | AAV-LK07 (See SEQ ID NO: 35 in |
| US20150376607) | US20150376607) |
| AAV-LK08 (See SEQ ID NO: 9 in | AAV-LK08 (See SEQ ID NO: 36 in |
| US20150376607) | US20150376607) |
| AAV-LK09 (See SEQ ID NO: 10 in | AAV-LK09 (See SEQ ID NO: 37 in |
| US20150376607) | US20150376607) |
| AAV-LK10 (See SEQ ID NO: 11 in | AAV-LK10 (See SEQ ID NO: 38 in |
| US20150376607) | US20150376607) |
| AAV-LK11 (See SEQ ID NO: 12 in | AAV-LK11 (See SEQ ID NO: 39 in |
| US20150376607) | US20150376607) |
| AAV-LK12 (See SEQ ID NO: 13 in | AAV-LK12 (See SEQ ID NO: 40 in |
| US20150376607) | US20150376607) |
| AAV-LK13 (See SEQ ID NO: 14 in | AAV-LK13 (See SEQ ID NO: 41 in |
| US20150376607) | US20150376607) |
| AAV-LK14 (See SEQ ID NO: 15 in | AAV-LK14 (See SEQ ID NO: 42 in |
| US20150376607) | US20150376607) |
| AAV-LK15 (See SEQ ID NO: 16 in | AAV-LK15 (See SEQ ID NO: 43 in |
| US20150376607) | US20150376607) |
| AAV-LK16 (See SEQ ID NO: 17 in | AAV-LK16 (See SEQ ID NO: 44 in |
| US20150376607) | US20150376607) |
| AAV-LK17 (See SEQ ID NO: 18 in | AAV-LK17 (See SEQ ID NO: 45 in |
| US20150376607) | US20150376607) |
| AAV-LK18 (See SEQ ID NO: 19 in | AAV-LK18 (See SEQ ID NO: 46 in |
| US20150376607) | US20150376607) |
| AAV-LK19 (See SEQ ID NO: 20 in | AAV-LK19 (See SEQ ID NO: 47 in |
| US20150376607) | US20150376607) |
| AAV-PAEC (See SEQ ID NO: 1 in | AAV-PAEC (See SEQ ID NO: 48 in |
| US20150376607) | US20150376607) |
| AAV-PAEC11 (See SEQ ID NO: 26 in | AAV-PAEC11 (See SEQ ID NO: 54 in |
| US20150376607) | US20150376607) |
| AAV-PAEC 12 (See SEQ ID NO: 27 in | AAV-PAEC 12 (See SEQ ID NO: 51 in |
| US20150376607) | US20150376607) |
| AAV-PAEC 13 (See SEQ ID NO: 28 in | AAV-PAEC 13 (See SEQ ID NO: 49 in |
| US20150376607) | US20150376607) |
| AAV-PAEC2 (See SEQ ID NO: 21 in | AAV-PAEC2 (See SEQ ID NO: 56 in |
| US20150376607) | US20150376607) |
| AAV-PAEC4 (See SEQ ID NO: 22 in | AAV-PAEC4 (See SEQ ID NO: 55 in |
| US20150376607) | US20150376607) |
| AAV-PAEC6 (See SEQ ID NO: 23 in | AAV-PAEC6 (See SEQ ID NO: 52 in |
| US20150376607) | US20150376607) |
| AAV-PAEC7 (See SEQ ID NO: 24 in | AAV-PAEC7 (See SEQ ID NO: 53 in |
| US20150376607) | US20150376607) |
| AAV-PAEC8 (See SEQ ID NO: 25 in | AAV-PAEC8 (See SEQ ID NO: 50 in |
| US20150376607) | US20150376607) |
| AAVpi.l (See SEQ ID NO: 28 in US20150315612) | AAVpi.l (See SEQ ID NO: 93 in |
| | US20150315612; AAVpi.2 408, see SEQ ID |
| | NO: 30 in US20150315612) |
| AAVpi.2 (See SEQ ID NO: 95 in | AAVpi.3 (See SEQ ID NO: 29 in |
| US20150315612) | US20150315612) |
| AAVpi.3 (See SEQ ID NO: 94 in | AAVrh.10 (See SEQ ID NO: 9 in |
| US20150315612) | US20150159173) |
| AAVrh.10 (See SEQ ID NO: 25 in | AAV44.2 (See SEQ ID NO: 59 in |
| US20150159173) | US20030138772) |
| AAVrh.10 (AAV44.2) (See SEQ ID NO: 81 in | AAV42.1B (See SEQ ID NO: 90 in |
| US20030138772) | US20030138772) |
| AAVrh.l2 (AAV42.1b) (See SEQ ID NO: 30 in | AAVrh.13 (See SEQ ID NO: 10 in |
| US20030138772) | US20150159173) |
| AAVrh.13 (See SEQ ID NO: 26 in | AAVrh.13 (See SEQ ID NO: 228 in |
| US20150159173) | US20150315612) |
| AAVrh.l3R (See SEQ ID NO: in US20150159173 | AAV42.3A (See SEQ ID NO: 87 in |
| | US20030138772) |
| AAVrh.l4 (AAV42.3a) (See SEQ ID NO: 32 in | AAV42.5A (See SEQ ID NO: 89 in |
| US20030138772) | US20030138772) |
| AAVrh.l7 (AAV42.5a) (See SEQ ID NO: 34 in | AAV42.5B (See SEQ ID NO: 91 in |
| US20030138772) | US20030138772) |
| AAVrh.l8 (AAV42.5b) (See SEQ ID NO: 29 in | AAV42.6B (See SEQ ID NO: 112 in |
| US20030138772) | US20030138772) |
| AAVrh.l9 (AAV42.6b) (See SEQ ID NO: 38 in | AAVrh.2 (See SEQ ID NO: 39 in |
| US20030138772) | US20150159173) |
| AAVrh.2 (See SEQ ID NO: 231 in | AAVrh.20 (See SEQ ID NO: 1 in |
| US20150315612) | US20150159173) |
| AAV42.10 (See SEQ ID NO: 106 in | AAVrh.21 (AAV42.10) (See SEQ ID NO: 35 |
| US20030138772) | in US20030138772) |
| AAV42.11 (See SEQ ID NO: 108 in | AAVrh.22 (AAV42.11) (See SEQ ID NO: 37 |
| US20030138772) | in US20030138772) |
| AAV42.12 (See SEQ ID NO: 113 in | AAVrh.23 (AAV42.12) (See SEQ ID NO: 58 |
| US20030138772) | in US20030138772) |
| AAV42.13 (See SEQ ID NO: 86 in | AAVrh.24 (AAV42.13) (See SEQ ID NO: 31 |
| US20030138772) | in US20030138772) |
| AAV42.15 (See SEQ ID NO: 84 in | AAVrh.25 (AAV42.15) (See SEQ ID NO: 28 |
| US20030138772) | in US20030138772) |
| AAVrh.2R (See SEQ ID NO: in US20150159173 | AAVrh.31 (AAV223.1) (See SEQ ID NO: 48 |
| | in US20030138772) |
| AAVC1 (See SEQ ID NO: 60 in US20030138772) | AAVrh.32 (AAVC1) (See SEQ ID NO: 19 in |
| | 446 US20030138772) |
| AAVrh.32/33 (See SEQ ID NO: 2 in | AAVrh.51 (AAV2-5) (See SEQ ID NO: 104 in |
| US20150159173) | US20150315612) |
| AAVrh.52 (AAV3-9) (See SEQ ID NO: 18 in | AAVrh.52 (AAV3-9) (See SEQ ID NO: 96 in |
| US20150315612) | US20150315612) |
| AAVrh.53 (See SEQ ID NO: in US20150315612) | AAVrh.53 (AAV3-11) (See SEQ ID NO: 17 in |
| | US20150315612) |
| AAVrh.53 (AAV3-11) (See SEQ ID NO: 186 in | AAVrh.54 (See SEQ ID NO: 40 in |
| US20150315612) | US20150315612) |
| AAVrh.54 (See SEQ ID NO: 49 in | |
| US20150159173 and SEQ ID NO: 116 in | |
| US20150315612) | |
| AAVrh.55 (See SEQ ID NO: 37 in | AAVrh.55 (AAV4-19) (See SEQ ID NO: 117 |
| US20150315612) | in US20150315612) |
| AAVrh.56 (See SEQ ID NO: 54 in | AAVrh.56 (See SEQ ID NO: 152 in |
| US20150315612) | US20150315612) |
| AAVrh.57 (See SEQ ID NO: in 497 | AAVrh.57 (See SEQ ID NO: 105 in |
| US20150315612 SEQ ID NO: 26 | US20150315612) |
| AAVrh.58 (See SEQ ID NO: 27 in | AAVrh.58 (See SEQ ID NO: 48 in |
| US20150315612) | US20150159173 and SEQ ID NO: 106 in |
| | US20150315612) |
| | AAVrh.58 (See SEQ ID NO: 232 in |
| | US20150315612) |
| AAVrh.59 (See SEQ ID NO: 42 in | AAVrh.59 (See SEQ ID NO: 110 in |
| US20150315612) | US20150315612) |
| AAVrh.60 (See SEQ ID NO: 31 in | AAVrh.60 (See SEQ ID NO: 120 in |
| US20150315612) | US20150315612) |
| AAVrh.61 (See SEQ ID NO: 107 in | AAVrh.61 (AAV2-3) (See SEQ ID NO: 21 in |
| US20150315612) | US20150315612) |
| AAVrh.62 (AAV2-15) (See SEQ ID NO: 33 in | AAVrh.62 (AAV2-15) (See SEQ ID NO: 114 |
| US20150315612) | in US20150315612) |
| AAVrh.64 (See SEQ ID NO: 15 in | AAVrh.64 (See SEQ ID NO: 43 in |
| US20150315612) | US20150159173 and SEQ ID NO: 99 in |
| | US20150315612) |
| | AAVrh.64 (See SEQ ID NO: 233 in |
| | US20150315612) |
| AAVRh.64Rl (See SEQ ID NO: in | AAVRh.64R2 (See SEQ ID NO: in |
| US20150159173 | US20150159173 |
| AAVrh.65 (See SEQ ID NO: 35 in | AAVrh.65 (See SEQ ID NO: 112 in |
| US20150315612) | US20150315612) |
| AAVrh.67 (See SEQ ID NO: 36 in | AAVrh.67 (See SEQ ID NO: 230 in |
| US20150315612) | US20150315612) |
| AAVrh.67 (See SEQ ID NO: 47 in | |
| US20150159173 and SEQ ID NO: 47 in | |
| US20150315612) | |
| AAVrh.68 (See SEQ ID NO: 16 in | AAVrh.68 (See SEQ ID NO: 100 in |
| US20150315612) | US20150315612) |
| AAVrh.69 (See SEQ ID NO: 39 in | AAVrh.69 (See SEQ ID NO: 119 in |
| US20150315612) | US20150315612) |
| AAVrh.70 (See SEQ ID NO: 20 in | AAVrh.70 (See SEQ ID NO: 98 in |
| US20150315612) | US20150315612) |
| AAVrh.71 (See SEQ ID NO: 162 in | AAVrh.72 (See SEQ ID NO: 9 in |
| US20150315612) | US20150315612) |
| AAVrh.73 (See SEQ ID NO: 5 in | AAVrh.74 (See SEQ ID NO: 6 in |
| US20150159173) | US20150159173) |
| AAVrh.8 (See SEQ ID NO: 41 in | AAVrh.8 (See SEQ ID NO: 235 in |
| US20150159173) | US20150315612) |
| AAVrh.8R (See SEQ ID NO: 9 in | AAVrh.8R A586R mutant (See SEQ ID NO: 10 |
| US20150159173, WO2015168666) | in WO2015168666) |
| AAVrh.8R R533A mutant (See SEQ ID NO: 11 in | BAAV (bovine AAV) (See SEQ ID NO: 8 in |
| WO2015168666) | U.S. Pat. No. 9,193,769) |
| BAAV (bovine AAV) (See SEQ ID NO: 10 in | BAAV (bovine AAV) (See SEQ ID NO: 4 in |
| U.S. Pat. No. 9,193,769) | U.S. Pat. No. 9,193,769) |
| BAAV (bovine AAV) (See SEQ ID NO: 2 in | BAAV (bovine AAV) (See SEQ ID NO: 6 in |
| U.S. Pat. No. 9,193,769) | U.S. Pat. No. 9,193,769) |
| BAAV (bovine AAV) (See SEQ ID NO: 1 in | BAAV (bovine AAV) (See SEQ ID NO: 5 in |
| U.S. Pat. No. 9,193,769) | U.S. Pat. No. 9,193,769) |
| BAAV (bovine AAV) (See SEQ ID NO: 3 in | BAAV (bovine AAV) (See SEQ ID NO: 11 in |
| U.S. Pat. No. 9,193,769) | U.S. Pat. No. 9,193,769) |
| BAAV (bovine AAV) (See SEQ ID NO: 5 in | BAAV (bovine AAV) (See SEQ ID NO: 6 in |
| U.S. Pat. No. 7,427,396) | U.S. Pat. No. 7,427,396) |
| BAAV (bovine AAV) (See SEQ ID NO: 7 in | BAAV (bovine AAV) (See SEQ ID NO: 9 in |
| U.S. Pat. No. 9,193,769) | U.S. Pat. No. 9,193,769) |
| BNP61 AAV (See SEQ ID NO: 1 in | BNP61 AAV (See SEQ ID NO: 2 in |
| US20150238550) | US20150238550) |
| BNP62 AAV (See SEQ ID NO: 3 in | BNP63 AAV (See SEQ ID NO: 4 in |
| US20150238550) | US20150238550) |
| caprine AAV (See SEQ ID NO: 3 in U.S. Pat. No. 7,427,396) | caprine AAV (See SEQ ID NO: 4 in |
| | U.S. Pat. No. 7,427,396) |
| true type AAV (ttAAV) (See SEQ ID NO: 2 in | AAAV (Avian AAV) (See SEQ ID NO: 12 in |
| WO2015121501) | U.S. Pat. No. 9,238,800) |
| AAAV (Avian AAV) (See SEQ ID NO: 2 in | AAAV (Avian AAV) (See SEQ ID NO: 6 in |
| U.S. Pat. No. 9,238,800) | U.S. Pat. No. 9,238,800) |
| AAAV (Avian AAV) (See SEQ ID NO: 4 in | AAAV (Avian AAV) (See SEQ ID NO: 8 in |
| U.S. Pat. No. 9,238,800) | U.S. Pat. No. 9,238,800) |
| AAAV (Avian AAV) (See SEQ ID NO: 14 in | AAAV (Avian AAV) (See SEQ ID NO: 10 in |
| U.S. Pat. No. 9,238,800) | U.S. Pat. No. 9,238,800) |
| AAAV (Avian AAV) (See SEQ ID NO: 15 in | AAAV (Avian AAV) (See SEQ ID NO: 5 in |
| U.S. Pat. No. 9,238,800) | U.S. Pat. No. 9,238,800) |
| AAAV (Avian AAV) (See SEQ ID NO: 9 in | AAAV (Avian AAV) (See SEQ ID NO: 3 in |
| U.S. Pat. No. 9,238,800) | U.S. Pat. No. 9,238,800) |
| AAAV (Avian AAV) (See SEQ ID NO: 7 in | AAAV (Avian AAV) (See SEQ ID NO: 11 in |
| U.S. Pat. No. 9,238,800) | U.S. Pat. No. 9,238,800) |
| AAAV (Avian AAV) (See SEQ ID NO: in | AAAV (Avian AAV) (See SEQ ID NO: 1 in |
| U.S. Pat. No. 9,238,800) | U.S. Pat. No. 9,238,800) |
| AAV Shuffle 100-1 (See SEQ ID NO: 23 in | AAV Shuffle 100-1 (See SEQ ID NO: 11 in |
| US20160017295) | US20160017295) |
| AAV Shuffle 100-2 (See SEQ ID NO: 37 in | AAV Shuffle 100-2 (See SEQ ID NO: 29 in |
| US20160017295) | US20160017295) |
| AAV Shuffle 100-3 (See SEQ ID NO: 24 in | AAV Shuffle 100-3 (See SEQ ID NO: 12 in |
| US20160017295) | US20160017295) |
| AAV Shuffle 100-7 (See SEQ ID NO: 25 in | AAV Shuffle 100-7 (See SEQ ID NO: 13 in |
| US20160017295) | US20160017295) |
| AAV Shuffle 10-2 (See SEQ ID NO: 34 in | AAV Shuffle 10-2 (See SEQ ID NO: 26 in |
| US20160017295) | US20160017295) |
| AAV Shuffle 10-6 (See SEQ ID NO: 35 in | AAV Shuffle 10-6 (See SEQ ID NO: 27 in |
| US20160017295) | US20160017295) |
| AAV Shuffle 10-8 (See SEQ ID NO: 36 in | AAV Shuffle 10-8 (See SEQ ID NO: 28 in |
| US20160017295) | US20160017295) |
| AAV SM 100-10 (See SEQ ID NO: 41 in | AAV SM 100-10 (See SEQ ID NO: 33 in |
| US20160017295) | US20160017295) |
| AAV SM 100-3 (See SEQ ID NO: 40 in | AAV SM 100-3 (See SEQ ID NO: 32 in |
| US20160017295) | US20160017295) |
| AAV SM 10-1 (See SEQ ID NO: 38 in | AAV SM 10-1 (See SEQ ID NO: 30 in |
| US20160017295) | US20160017295) |
| AAV SM 10-2 (See SEQ ID NO: 10 in | AAV SM 10-2 (See SEQ ID NO: 22 in |
| US20160017295) | US20160017295) |
| AAV SM 10-8 (See SEQ ID NO: 39 in | AAV SM 10-8 (See SEQ ID NO: 31 in |
| US20160017295) | US20160017295) |
| AAV CBr-7.1 (See SEQ ID NO: 4 in | AAV CBr-7.1 (See SEQ ID NO: 54 in |
| WO2016065001) | WO2016065001) |
| AAV CBr-7.10 (See SEQ ID NO: 11 in | AAV CBr-7.10 (See SEQ ID NO: 61 in |
| WO2016065001) | WO2016065001) |
| AAV CBr-7.2 (See SEQ ID NO: 5 in | AAV CBr-7.2 (See SEQ ID NO: 55 in |
| WO2016065001) | WO2016065001) |
| AAV CBr-7.3 (See SEQ ID NO: 6 in | AAV CBr-7.3 (See SEQ ID NO: 56 in |
| WO2016065001) | WO2016065001) |
| AAV CBr-7.4 (See SEQ ID NO: 7 in | AAV CBr-7.4 (See SEQ ID NO: 57 in |
| WO2016065001) | WO2016065001) |
| AAV CBr-7.5 (See SEQ ID NO: 8 in | AAV CHt-6.6 (See SEQ ID NO: 35 in |
| WO2016065001) | WO2016065001) |
| AAV CHt-6.6 (See SEQ ID NO: 85 in | AAV CHt-6.7 (See SEQ ID NO: 36 in |
| WO2016065001) | WO2016065001) |
| AAV CHt-6.7 (See SEQ ID NO: 86 in | AAV CHt-6.8 (See SEQ ID NO: 37 in |
| WO2016065001) | WO2016065001) |
| AAV CHt-6.8 (See SEQ ID NO: 87 in | AAV CHt-Pl (See SEQ ID NO: 29 in |
| WO2016065001) | WO2016065001) |
| AAV CHt-Pl (See SEQ ID NO: 79 in | AAV CHt-P2 (See SEQ ID NO: 1 in |
| WO2016065001) | WO2016065001) |
| AAV CHt-P2 (See SEQ ID NO: 51 in | AAV CHt-P5 (See SEQ ID NO: 2 in |
| WO2016065001) | WO2016065001) |
| AAV CHt-P5 (See SEQ ID NO: 52 in | AAV CHt-P6 (See SEQ ID NO: 30 in |
| WO2016065001) | WO2016065001) |
| AAV CHt-P6 (See SEQ ID NO: 80 in | AAV CHt-P8 (See SEQ ID NO: 31 in |
| WO2016065001) | WO2016065001) |
| AAV CHt-P8 (See SEQ ID NO: 81 in | AAV CHt-P9 (See SEQ ID NO: 3 in |
| WO2016065001) | WO2016065001) |
| AAV CHt-P9 (See SEQ ID NO: 53 in | AAV CKd-1 (See SEQ ID NO: 57 in |
| WO2016065001) | U.S. Pat. No. 8,734,809) |
| AAV CKd-1 (See SEQ ID NO: 131 in | AAV CKd-10 (See SEQ ID NO: 58 in |
| U.S. Pat. No. 8,734,809) | U.S. Pat. No. 8,734,809) |
| AAV CKd-10 (See SEQ ID NO: 132 in | AAV CKd-2 (See SEQ ID NO: 59 in |
| U.S. Pat. No. 8,734,809) | U.S. Pat. No. 8,734,809) |
| AAV CKd-2 (See SEQ ID NO: 133 in | AAV CKd-3 (See SEQ ID NO: 60 in |
| U.S. Pat. No. 8,734,809) | U.S. Pat. No. 8,734,809) |
| AAV CKd-3 (See SEQ ID NO: 134 in | AAV CKd-4 (See SEQ ID NO: 61 in |
| U.S. Pat. No. 8,734,809) | U.S. Pat. No. 8,734,809) |
| AAV CKd-4 (See SEQ ID NO: 135 in | AAV CKd-6 (See SEQ ID NO: 62 in |
| U.S. Pat. No. 8,734,809) | U.S. Pat. No. 8,734,809) |
| AAV CKd-6 (See SEQ ID NO: 136 in | AAV CKd-7 (See SEQ ID NO: 63 in |
| U.S. Pat. No. 8,734,809) | U.S. Pat. No. 8,734,809) |
| AAV CKd-7 (See SEQ ID NO: 137 in | AAV CKd-8 (See SEQ ID NO: 64 in |
| U.S. Pat. No. 8,734,809) | U.S. Pat. No. 8,734,809) |
| AAV CKd-8 (See SEQ ID NO: 138 in | AAV CKd-B 1 (See SEQ ID NO: 73 in |
| U.S. Pat. No. 8,734,809) | U.S. Pat. No. 8,734,809) |
| AAV CKd-B 1 (See SEQ ID NO: 147 in | AAV CKd-B2 (See SEQ ID NO: 74 in |
| U.S. Pat. No. 8,734,809) | U.S. Pat. No. 8,734,809) |
| AAV CKd-B2 (See SEQ ID NO: 148 in | AAV CKd-B3 (See SEQ ID NO: 75 in |
| U.S. Pat. No. 8,734,809) | U.S. Pat. No. 8,734,809) |
| AAV CKd-B3 (See SEQ ID NO: in U.S. Pat. No. 8,734,809 | AAV CKd-B3 (See SEQ ID NO: 149 in |
| | U.S. Pat. No. 8,734,809) |
| AAV CLv-1 (See SEQ ID NO: 65 in U.S. Pat. No. 8,734,809) | AAV CLv-1 (See SEQ ID NO: 139 in |
| | U.S. Pat. No. 8,734,809) |
| AAV CLvl-1 (See SEQ ID NO: 171 in | AAV Civ 1-10 (See SEQ ID NO: 178 in |
| U.S. Pat. No. 8,734,809) | U.S. Pat. No. 8,734,809) |
| AAV CLvl-2 (See SEQ ID NO: 172 in | AAV CLv-12 (See SEQ ID NO: 66 in |
| U.S. Pat. No. 8,734,809) | U.S. Pat. No. 8,734,809) |
| AAV CLv-12 (See SEQ ID NO: 140 in | AAV CLvl-3 (See SEQ ID NO: 173 in |
| U.S. Pat. No. 8,734,809) | U.S. Pat. No. 8,734,809) |
| AAV CLv-13 (See SEQ ID NO: 67 in | AAV CLv-13 (See SEQ ID NO: 141 in |
| U.S. Pat. No. 8,734,809) | U.S. Pat. No. 8,734,809) |
| AAV CLvl-4 (See SEQ ID NO: 174 in | AAV Civ 1-7 (See SEQ ID NO: 175 in |
| U.S. Pat. No. 8,734,809) | U.S. Pat. No. 8,734,809) |
| AAV Civ 1-8 (See SEQ ID NO: 176 in | AAV Civ 1-9 (See SEQ ID NO: 177 in |
| U.S. Pat. No. 8,734,809) | U.S. Pat. No. 8,734,809) |
| AAV CLv-2 (See SEQ ID NO: 68 in U.S. Pat. No. 8,734,809) | AAV CLv-2 (See SEQ ID NO: 142 in |
| | U.S. Pat. No. 8,734,809) |
| AAV CLv-3 (See SEQ ID NO: 69 in U.S. Pat. No. 8,734,809) | AAV CLv-3 (See SEQ ID NO: 143 in |
| | U.S. Pat. No. 8,734,809) |
| AAV CLv-4 (See SEQ ID NO: 70 in U.S. Pat. No. 8,734,809) | AAV CLv-4 (See SEQ ID NO: 144 in |
| | U.S. Pat. No. 8,734,809) |
| AAV CLv-6 (See SEQ ID NO: 71 in U.S. Pat. No. 8,734,809) | AAV CLv-6 (See SEQ ID NO: 145 in |
| | U.S. Pat. No. 8,734,809) |
| AAV CLv-8 (See SEQ ID NO: 72 in U.S. Pat. No. 8,734,809) | AAV CLv-8 (See SEQ ID NO: 146 in |
| | U.S. Pat. No. 8,734,809) |
| AAV CLv-Dl (See SEQ ID NO: 22 in | AAV CLv-Dl (See SEQ ID NO: 96 in |
| U.S. Pat. No. 8,734,809) | U.S. Pat. No. 8,734,809) |
| AAV CLv-D2 (See SEQ ID NO: 23 in | AAV CLv-D2 (See SEQ ID NO: 97 in |
| U.S. Pat. No. 8,734,809) | U.S. Pat. No. 8,734,809) |
| AAV CLv-D3 (See SEQ ID NO: 24 in | AAV CLv-D3 (See SEQ ID NO: 98 in |
| U.S. Pat. No. 8,734,809) | U.S. Pat. No. 8,734,809) |
| AAV CLv-D4 (See SEQ ID NO: 25 in | AAV CLv-D4 (See SEQ ID NO: 99 in |
| U.S. Pat. No. 8,734,809) | U.S. Pat. No. 8,734,809) |
| AAV CLv-D5 (See SEQ ID NO: 26 in | AAV CLv-D5 (See SEQ ID NO: 100 in |
| U.S. Pat. No. 8,734,809) | U.S. Pat. No. 8,734,809) |
| AAV CLv-D6 (See SEQ ID NO: 27 in | AAV CLv-D6 (See SEQ ID NO: 101 in |
| U.S. Pat. No. 8,734,809) | U.S. Pat. No. 8,734,809) |
| AAV CLv-D7 (See SEQ ID NO: 28 in | AAV CLv-D7 (See SEQ ID NO: 102 in |
| U.S. Pat. No. 8,734,809) | U.S. Pat. No. 8,734,809) |
| AAV CLv-D8 (See SEQ ID NO: 29 in | AAV CLv-D8 (See SEQ ID NO: 103 in |
| U.S. Pat. No. 8,734,809) | U.S. Pat. No. 8,734,809); AAV CLv-Kl 762, see SEQ ID |
| | NO: 18 in WO2016065001) |
| AAV CLv-Kl (See SEQ ID NO: 68 in | AAV CLv-K3 (See SEQ ID NO: 19 in |
| WO2016065001) | WO2016065001) |
| AAV CLv-K3 (See SEQ ID NO: 69 in | AAV CLv-K6 (See SEQ ID NO: 20 in |
| WO2016065001) | WO2016065001) |
| AAV CLv-K6 (See SEQ ID NO: 70 in | AAV CLv-L4 (See SEQ ID NO: 15 in |
| WO2016065001) | WO2016065001) |
| AAV CLv-L4 (See SEQ ID NO: 65 in | AAV CLv-L5 (See SEQ ID NO: 16 in |
| WO2016065001) | WO2016065001) |
| AAV CLv-L5 (See SEQ ID NO: 66 in | AAV CLv-L6 (See SEQ ID NO: 17 in |
| WO2016065001) | WO2016065001) |
| AAV CLv-L6 (See SEQ ID NO: 67 in | AAV CLv-Ml (See SEQ ID NO: 21 in |
| WO2016065001) | WO2016065001) |
| AAV CLv-Ml (See SEQ ID NO: 71 in | AAV CLv-Mll (See SEQ ID NO: 22 in |
| WO2016065001) | WO2016065001) |
| AAV CLv-Ml 1 (See SEQ ID NO: 72 in | AAV CLv-M2 (See SEQ ID NO: 23 in |
| WO2016065001) | WO2016065001) |
| AAV CLv-M2 (See SEQ ID NO: 73 in | AAV CLv-M5 (See SEQ ID NO: 24 in |
| WO2016065001) | WO2016065001) |
| AAV CLv-M5 (See SEQ ID NO: 74 in | AAV CLv-M6 (See SEQ ID NO: 25 in |
| WO2016065001) | WO2016065001) |
| AAV CLv-M6 (See SEQ ID NO: 75 in | AAV CLv-M7 (See SEQ ID NO: 26 in |
| WO2016065001) | WO2016065001) |
| AAV CLv-M7 (See SEQ ID NO: 76 in | AAV CLv-M8 (See SEQ ID NO: 27 in |
| WO2016065001) | WO2016065001) |
| AAV CLv-M8 (See SEQ ID NO: 77 in | AAV CLv-M9 (See SEQ ID NO: 28 in |
| WO2016065001) | WO2016065001) |
| AAV CLv-M9 (See SEQ ID NO: 78 in | AAV CLv-Rl (See SEQ ID NO: 30 in |
| WO2016065001) | U.S. Pat. No. 8,734,809) |
| AAV CLv-Rl (See SEQ ID NO: 104 in | AAV CLv-R2 (See SEQ ID NO: 31 in |
| U.S. Pat. No. 8,734,809) | U.S. Pat. No. 8,734,809) |
| AAV CLv-R2 (See SEQ ID NO: 105 in | AAV CLv-R3 (See SEQ ID NO: 32 in |
| U.S. Pat. No. 8,734,809) | U.S. Pat. No. 8,734,809) |
| AAV CLv-R3 (See SEQ ID NO: 106 in | AAV CLv-R4 (See SEQ ID NO: 33 in |
| U.S. Pat. No. 8,734,809) | U.S. Pat. No. 8,734,809) |
| AAV CLv-R4 (See SEQ ID NO: 107 in | AAV CLv-R5 (See SEQ ID NO: 34 in |
| U.S. Pat. No. 8,734,809) | U.S. Pat. No. 8,734,809) |
| AAV CLv-R5 (See SEQ ID NO: 108 in | AAV CLv-R6 (See SEQ ID NO: 35 in |
| U.S. Pat. No. 8,734,809) | U.S. Pat. No. 8,734,809) |
| AAV CLv-R6 (See SEQ ID NO: 109 in | AAV CLv-R7 (See SEQ ID NO: 110 in |
| U.S. Pat. No. 8,734,809); AAV CLv-R7 802 (see SEQ ID NO: | U.S. Pat. No. 8,734,809) |
| 36 in U.S. Pat. No. 8,734,809) | |
| AAV CLv-R8 (See SEQ ID NO: 37 in | AAV CLv-R8 (See SEQ ID NO: 111 in |
| U.S. Pat. No. 8,734,809) | U.S. Pat. No. 8,734,809) |
| AAV CLv-R9 (See SEQ ID NO: 38 in | AAV CLv-R9 (See SEQ ID NO: 112 in |
| U.S. Pat. No. 8,734,809) | U.S. Pat. No. 8,734,809) |
| AAV CSp-1 (See SEQ ID NO: 45 in U.S. Pat. No. 8,734,809) | AAV CSp-1 (See SEQ ID NO: 119 in |
| | U.S. Pat. No. 8,734,809) |
| AAV CSp-10 (See SEQ ID NO: 46 in U.S. Pat. No. 8,734,809) | AAV CSp-10 (See SEQ ID NO: 120 in |
| | U.S. Pat. No. 8,734,809) |
| AAV CSp-11 (See SEQ ID NO: 47 in U.S. Pat. No. 8,734,809) | AAV CSp-11 (See SEQ ID NO: 121 in |
| | U.S. Pat. No. 8,734,809) |
| AAV CSp-2 (See SEQ ID NO: 48 in U.S. Pat. No. 8,734,809) | AAV CSp-2 (See SEQ ID NO: 122 in |
| | U.S. Pat. No. 8,734,809) |
| AAV CSp-3 (See SEQ ID NO: 49 in U.S. Pat. No. 8,734,809) | AAV CSp-3 (See SEQ ID NO: 123 in |
| | U.S. Pat. No. 8,734,809) |
| AAV CSp-4 (See SEQ ID NO: 50 in U.S. Pat. No. 8,734,809) | AAV CSp-4 (See SEQ ID NO: 124 in |
| | U.S. Pat. No. 8,734,809) |
| AAV CSp-6 (See SEQ ID NO: 51 in U.S. Pat. No. 8,734,809) | AAV CSp-6 (See SEQ ID NO: 125 in |
| | U.S. Pat. No. 8,734,809) |
| AAV CSp-7 (See SEQ ID NO: 52 in U.S. Pat. No. 8,734,809) | AAV CSp-7 (See SEQ ID NO: 126 in |
| | U.S. Pat. No. 8,734,809) |
| AAV CSp-8 (See SEQ ID NO: 53 in U.S. Pat. No. 8,734,809) | AAV CSp-8 (See SEQ ID NO: 127 in |
| | U.S. Pat. No. 8,734,809) |
| AAV CSp-8.10 (See SEQ ID NO: 38 in | AAV CSp-8.10 (See SEQ ID NO: 88 in |
| WO2016065001) | WO2016065001) |
| AAV CSp-8.2 (See SEQ ID NO: 39 in | AAV CSp-8.2 (See SEQ ID NO: 89 in |
| WO2016065001) | WO2016065001) |
| AAV CSp-8.4 (See SEQ ID NO: 40 in | AAV CSp-8.4 (See SEQ ID NO: 90 in |
| WO2016065001) | WO2016065001) |
| AAV CSp-8.5 (See SEQ ID NO: 41 in | AAV CSp-8.5 (See SEQ ID NO: 91 in |
| WO2016065001) | WO2016065001) |
| AAV CSp-8.6 (See SEQ ID NO: 42 in | AAV CSp-8.6 (See SEQ ID NO: 92 in |
| WO2016065001) | WO2016065001) |
| AAV CSp-8.7 (See SEQ ID NO: 43 in | AAV CSp-8.7 (See SEQ ID NO: 93 in |
| WO2016065001) | WO2016065001) |
| AAV CSp-8.8 (See SEQ ID NO: 44 in | AAV CSp-8.8 (See SEQ ID NO: 94 in |
| WO2016065001) | WO2016065001) |
| AAV CSp-8.9 (See SEQ ID NO: 45 in | AAV CSp-8.9 (See SEQ ID NO: 95 in |
| WO2016065001) | WO2016065001) |
| AAV CSp-9 842 (See SEQ ID NO: 54 in | AAV CSp-9 (See SEQ ID NO: 128 in |
| U.S. Pat. No. 8,734,809) | U.S. Pat. No. 8,734,809) |
| AAV.hu.48R3 (See SEQ ID NO: 183 in | AAV.VR-355 (See SEQ ID NO: 181 in |
| U.S. Pat. No. 8,734,809) | U.S. Pat. No. 8,734,809) |
| AAV3B (See SEQ ID NO: 48 in WO2016065001) | AAV3B (See SEQ ID NO: 98 in |
| | WO2016065001) |
| AAV4 (See SEQ ID NO: 49 in WO2016065001) | AAV4 (See SEQ ID NO: 99 in |
| | WO2016065001) |
| AAV5 (See SEQ ID NO: 50 in WO2016065001) | AAV5 (See SEQ ID NO: 100 in |
| | WO2016065001) |
| AAVF1/HSC1 (See SEQ ID NO: 20 in | AAVF1/HSC1 (See SEQ ID NO: 2 in |
| WO2016049230) | WO2016049230) |
| AAVF11/HSC11 (See SEQ ID NO: 26 in | AAVF11/HSC11 (See SEQ ID NO: 4 in |
| WO2016049230) | WO2016049230) |
| AAVF12/HSC12 (See SEQ ID NO: 30 in | AAVF12/HSC12 (See SEQ ID NO: 12 in |
| WO2016049230) | WO2016049230) |
| AAVF13/HSC13 (See SEQ ID NO: 31 in | AAVF13/HSC13 (See SEQ ID NO: 14 in |
| WO2016049230) | WO2016049230) |
| AAVF14/HSC14 (See SEQ ID NO: 32 in | AAVF14/HSC14 (See SEQ ID NO: 15 in |
| WO2016049230) | WO2016049230) |
| AAVF15/HSC15 (See SEQ ID NO: 33 in | AAVF15/HSC15 (See SEQ ID NO: 16 in |
| WO2016049230) | WO2016049230) |
| AAVF16/HSC16 (See SEQ ID NO: 34 in | AAVF16/HSC16 (See SEQ ID NO: 17 in |
| WO2016049230) | WO2016049230) |
| AAVF17/HSC17 (See SEQ ID NO: 35 in | AAVF17/HSC17 (See SEQ ID NO: 13 in |
| WO2016049230) | WO2016049230) |
| AAVF2/HSC2 (See SEQ ID NO: 21 in | AAVF2/HSC2 (See SEQ ID NO: 3 in |
| WO2016049230) | WO2016049230) |
| AAVF3/HSC3 (See SEQ ID NO: 22 in | AAVF3/HSC3 (See SEQ ID NO: 5 in |
| WO2016049230) | WO2016049230) |
| AAVF4/HSC4 (See SEQ ID NO: 23 in | AAVF4/HSC4 (See SEQ ID NO: 6 in |
| WO2016049230) | WO2016049230) |
| AAVF5/HSC5 (See SEQ ID NO: 25 in | AAVF5/HSC5 (See SEQ ID NO: 11 in |
| WO2016049230) | WO2016049230) |
| AAVF6/HSC6 (See SEQ ID NO: 24 in | AAVF6/HSC6 (See SEQ ID NO: 7 in |
| WO2016049230) | WO2016049230) |
| AAVF7/HSC7 (See SEQ ID NO: 27 in | AAVF7/HSC7 (See SEQ ID NO: 8 in |
| WO2016049230) | WO2016049230) |
| AAVF8/HSC8 (See SEQ ID NO: 28 in | AAVF8/HSC8 (See SEQ ID NO: 9 in |
| WO2016049230) | WO2016049230) |
| AAVF9/HSC9 (See SEQ ID NO: 10 in | AAVF9/HSC9 882 (see SEQ ID NO: 29 in |
| WO2016049230) | WO2016049230) |

In one embodiment, the AAV vector (also referred to as a rAAV virion) as disclosed herein comprises a capsid protein from any of those disclosed in WO2019/241324, which is specifically incorporated herein in its entirety by reference. In some embodiments, the rAAV vector comprises a liver specific capsid, e.g., a liver specific capsid selected from XL32 and XL32.1, as disclosed in WO2019/241324, which is incorporated herein in its entirety by reference. In some embodiments, the rAAV vector is a AAVXL32 or AAVXL32.1 as disclosed in WO2019/241324, which is incorporated herein in its entirety by reference.

Exemplary chimeric or variant capsid proteins that can be used as the AAV capsid in the rAAV vector described herein can be selected from Table 2 from U.S. provisional application 62,937,556, filed on Nov. 19, 2019 (PCT/US20/61223, filed on Nov. 19, 2020; WO 2021/102107), which is specifically incorporated herein by reference or can be used with any combination with wild type capsid proteins and/or other chimeric or variant capsid proteins now known or later identified and each is incorporated herein. In some embodiments, the rAAV vector encompassed for use is a chimeric vector, e.g., as disclosed in 9,012,224 and U.S. Pat. No. 7,892,809, which are incorporated herein in their entirety by reference.

In some embodiments, the rAAV vector is a haploid rAAV vector, as disclosed in US application US2018/0371496 and PCT/US18/22725, or polyploid rAAV vector, e.g., as disclosed in PCT/US2018/044632 filed on Jul. 31, 2018 and in U.S. application Ser. No. 16/151,110, each of which are incorporated herein in their entirety by reference. In some embodiments, the rAAV vector is a rAAV3 vector, as disclosed in 9,012,224 and WO 2017/106236 which are incorporated herein in their entirety by reference.

In a particular embodiment, the rAAV is a AAVXL32 or AAVXL32.1 AAV vector as disclosed in WO2019/241324, which is incorporated herein in its entirety by reference. In some embodiments, the rAAV vector comprises a capsid disclosed in WO2019241324A1, or International Patent application PCT/US2019/036676, which are incorporated herein in their entirety by reference. In some embodiments, the AAV vector is a AAV8 vector or a rational haploid comprising an AAV8 capsid protein. In some embodiments, the recombinant AAV vector is a chimeric AAV vector, haploid AAV vector, a hybrid AAV vector or polyploid AAV vector. In some embodiments, the recombinant AAV vector is a rational haploid vector, a mosaic AAV vector, a chemically modified AAV vector, or a AAV vector from any AAV serotypes, for example, from any AAV serotype disclosed in Table 1 as disclosed in International Applications WO2020/102645, and WO2020/102667, each of which are incorporated herein in their entirety.

In an embodiment, an rAAV vector useful in the treatment of Hemophilia A as disclosed herein is an AAV3b capsid. AAV3b capsids encompassed for use are described in 2017/106236, and 9,012,224 and 7,892,809, and International application PCT/US19/61653, filed Nov. 15, 2019, and International Applications WO2020/102645, and WO2020/102667, each of which are incorporated herein in their entirety. In addition, AAV3b capsids of the AAV vector for use according to the methods as disclosed herein are disclosed in International Patent Applications WO 2020/102645 and WO2021102107, which are incorporated herein in its entirety by reference herein.

In some embodiments, the AAV3b capsid comprises SEQ ID NO: 44 as disclosed in International Patent Applications WO 2020/102645 and WO2021102107. In an embodiment, the AAV capsid used in the treatment of Hemophilia A can be a modified AAV capsid that is derived in whole or in part from the AAV capsid set forth in SEQ ID NO: 44. In some embodiments, the amino acids from an AAV3b capsid as set forth in SEQ ID NO: 44 can be, or are substituted with amino acids from another capsid of a different AAV serotype, wherein the substituted and/or inserted amino acids can be from any AAV serotype, and can include either naturally occurring or partially or completely synthetic amino acids.

In another embodiment, an AAV capsid used in the treatment of Hemophilia A is an AAV3b265D capsid. In this particular embodiment, an AAV3b265D capsid comprises a modification in the amino acid sequence of the two-fold axis loop of an AAV3b capsid via replacement of amino acid G265 of the AAV3b capsid with D265. In some embodiments, an AAV3b265D capsid comprises SEQ ID NO: 46. However, the modified virus capsids of the invention are not limited to AAV capsids set forth in SEQ ID NO: 46 as set forth in International Patent Applications WO 2020/102645 and WO2021102107. In some embodiments, the amino acids from AAV3b265D as set forth in SEQ ID NO. 46 can be, or are substituted with amino acids from a capsid from an AAV of a different serotype, wherein the substituted and/or inserted amino acids can be from any AAV serotype, and can include either naturally occurring or partially or completely synthetic amino acids.

In another embodiment an rAAV vector useful in the treatment of Hemophilia A as disclosed herein is an AAV3b265D549A capsid. In this particular embodiment, an AAV3b265D549A capsid comprises a modification in the amino acid sequence of the two-fold axis loop of an AAV3b capsid via replacement of amino acid G265 of the AAV3b capsid with D265 and replacement of amino acid T549 of the AAV3b capsid with A549. In some embodiments, an AAV3b265D549A capsid comprises SEQ ID NO: 50 as disclosed herein International Patent Applications WO 2020/102645 and WO2021102107. However, the modified virus capsids of the invention are not limited to AAV capsids set forth in SEQ ID NO: 50. In some embodiments, the amino acids from AAV3b265D549A as set forth in SEQ ID NO: 50 can be, or are substituted with amino acids from a capsid from an AAV of a different serotype, wherein the substituted and/or inserted amino acids can be from any AAV serotype, and can include either naturally occurring or partially or completely synthetic amino acids. In some embodiments, the amino acids from AAV3bSASTG (i.e., a AAV3b capsid comprising Q263A/T265 mutations) can be, or are substituted with amino acids from a capsid from an AAV of a different serotype, wherein the substituted and/or inserted amino acids can be from any AAV serotype, and can include either naturally occurring or partially or completely synthetic amino acids.

In another embodiment, an rAAV vector useful in the treatment of Hemophilia A as disclosed herein is an AAV3b549A capsid. In this particular embodiment, an AAV3b549A capsid comprises a modification in the amino acid sequence of the two-fold axis loop of an AAV3b capsid via replacement of amino acid T549 of the AAV3b capsid with A549. In some embodiments, an AAV3b549A capsid comprises SEQ ID NO: 52 as disclosed herein International Patent Applications WO 2020/102645 and WO2021102107. However, the modified virus capsids of the invention are not limited to AAV capsids set forth in SEQ ID NO: 52. In some embodiments, the amino acids from AAV3b549A as set forth in SEQ ID NO: 52 can be, or are substituted with amino acids from a capsid from an AAV of a different serotype, wherein the substituted and/or inserted amino acids can be from any AAV serotype, and can include either naturally occurring or partially or completely synthetic amino acids.

In another embodiment, an rAAV vector useful in the treatment of Hemophilia A as disclosed herein is an AAV3bQ263Y capsid. In this particular embodiment, an AAV3bQ263Y capsid comprises a modification in the amino acid sequence of the two-fold axis loop of an AAV3b capsid via replacement of amino acid Q263 of the AAV3b capsid with Y263. In some embodiments, an AAV3b549A capsid comprises SEQ ID NO: 54 as disclosed herein International Patent Applications WO 2020/102645 and WO2021102107. However, the modified virus capsids of the invention are not limited to AAV capsids set forth in SEQ ID NO: 54. In some embodiments, the amino acids from AAV3bQ263Y as set forth in SEQ ID NO: 54 can be, or are substituted with amino acids from a capsid from an AAV of a different serotype, wherein the substituted and/or inserted amino acids can be from any AAV serotype, and can include either naturally occurring or partially or completely synthetic amino acids.

In another embodiment, an rAAV vector useful in the treatment of Hemophilia A as disclosed herein is AAV3bSASTG serotype or comprises a AAV3bSASTG capsid. In this particular embodiment, an AAV3bSASTG capsid comprises a modification in the amino acid sequence to comprise a SASTG mutation, in particular, the AAV3b capsid was modified to resemble AAV2 Q263A/T265 subvariant by introducing these modifications at similar positions in the AAV3b capsid (as disclosed in Messina E L, et al., Adeno-associated viral vectors based on serotype 3b use components of the fibroblast growth factor receptor signaling complex for efficient transduction. Hum. Gene Ther. 2012 October: 23(10):1031-4, Piacentino III, Valentino, et al. “X-linked inhibitor of apoptosis protein-mediated attenuation of apoptosis, using a novel cardiac-enhanced adeno-associated viral vector.” Human gene therapy 23.6 (2012): 635-646. which are both incorporated herein in their entirety by reference). Accordingly, in some embodiments, an rAAV vector useful in the treatment of Hemophilia A as disclosed herein is AAV3bSASTG serotype or comprises a AAV3bSASTG capsid comprising a AAV3b Q263A/T265 capsid. In some embodiments, the amino acids from AAV3bSASTG can be, or are substituted with amino acids from a capsid from an AAV of a different serotype, wherein the substituted and/or inserted amino acids can be from any AAV serotype, and can include either naturally occurring or partially or completely synthetic amino acids.

One can target desired tissues using the appropriate capsids. For example, the central nervous system using AAV9 or a rhesus capsid or a rational haploid using at least one of a AAV9 or Rhesus viral protein. One can target the muscle using myo AAV, see, e.g., WO2019/2071323 and WO2022/020616, which are incorporated herein in their entirety by reference.

In order to facilitate their introduction into a cell, an rAAV vector genome useful in the invention are recombinant nucleic acid constructs that include (1) a heterologous sequence to be expressed (in one embodiment, a polynucleotide encoding a FVIII polypeptide) and (2) viral sequence elements that facilitate integration and expression of the heterologous genes. The viral sequence elements may include those sequences of an AAV vector genome that are required in cis for replication and packaging (e.g., functional ITRs) of the DNA into an AAV capsid. In an embodiment, the heterologous gene encodes FVIII, which is useful for correcting a FVIII-deficiency in a patient suffering from Hemophilia A. In an embodiment, such an rAAV vector genome may also contain marker or reporter genes. In an embodiment, an rAAV vector genome can have one or more of the AAV3b wild-type (WT) cis genes replaced or deleted in whole or in part, but retain functional flanking ITR sequences.

### Methods of Treatment

The rAAV vectors, codon-optimized nucleic acids encoding FVIII protein, and expression cassettes described herein can be used in methods to treat Hemophilia A. Aspects of the invention relate to the treatment of disease (e.g., Hemophilia A) by administration of the rAAV vectors, codon-optimized nucleic acids or expression cassettes (e.g., contained within in a pharmaceutical composition) disclosed herein, to a subject in need thereof for therapeutic expression of the codon-optimized nucleic acids encoding a FVIII polypeptide in the subject.

In any embodiment of the methods as disclosed herein, a FVIII polypeptide suitable for use in the therapeutic method includes those proteins encoded by the codon optimized FVIII nucleic acids described herein. In some embodiments of the methods and compositions as disclosed herein, the FVIII polypeptide is encoded by a codon optimized FVIII nucleic acid sequence. In some embodiments of the methods and compositions as disclosed herein, the FVIII polypeptide is encoded by a codon optimized FVIII nucleic sequence, for example, a nucleic acid with the sequence set forth in any of: SEQ ID NO: 1-18 (or a subset thereof such as SEQ ID NOs 1, 2, 4, 5, 7-9, 11-15 or 18; SEQ ID NOs 4, 5, 7, 12-15 or 18; SEQ ID NOs 4, 5, 13, or 15; or SEQ ID NOs 4 or 5), or a nucleic acid sequence having at least 90%, 91%, 92%, 93%, 94%, 95%, 96%, 97%, or 98%, or 99% sequence identity thereto, which encode a FVIII polypeptide, e.g., which lacks a B domain and, where amino acid at position 355 is Q (355Q) and amino acid at position 581 is Q (581Q), as compared to the wild type FVIII protein).

In one embodiment, the R355Q and R581Q point mutations in FVIII correspond to R336Q and R562Q if the FVIII is lacking its endogenous N-terminal signal peptide. Accordingly, in one embodiment, the rAAV comprises nucleic acid sequences of the invention, or fragment thereof that encodes FVIII polypeptide that is devoid of B domain and N terminal signal peptide, and that has Q at amino acid position 336 and position 562.

In some embodiments of the methods and compositions as disclosed herein, a rAAV vector as described herein transduces the liver of a subject and secretes the FVIII polypeptide into the blood.

In some embodiments, upon administration, the AAV vector selectively expresses and secretes FVIII from transduced hepatocytes.

In any embodiment of the methods as disclosed herein, administration of a AAV vector expressing the FVIII polypeptide can be by any suitable method including by systemic administration (e.g., intravenous administration, intra-arterial administration, and/or intra-peritoneal administration) and local administration (e.g, to the liver). Exemplary modes of administration include oral, rectal, transmucosal, intranasal, inhalation (e.g., via an aerosol), buccal (e.g., sublingual), vaginal, intrathecal, intraocular, transdermal, in utero (or in ovo), parenteral (e.g., intravenous, subcutaneous, intradermal, intramuscular, intradermal, intrapleural, intracerebral, and intraarticular), topical (e.g., to both skin and mucosal surfaces, including airway surfaces, and transdermal administration), intralymphatic, and the like, as well as direct tissue or organ injection (e.g., to liver, skeletal muscle, cardiac muscle, diaphragm muscle or brain). In some embodiments, administration is directly to the liver. The most suitable route in any given case will depend on the nature and severity of the condition being treated and/or prevented and on the nature of the particular vector that is being used.

In any embodiment of the methods as disclosed herein, the rAAV vectors and/or rAAV genome are administered to the skeletal muscle, liver, diaphragm, costal, and/or cardiac muscle cells of a subject. For example, a conventional syringe and needle can be used to inject a rAAV virion suspension into a subject. Parenteral administration of a the rAAV vectors and/or rAAV genome, by injection can be performed, for example, by bolus injection or continuous infusion. Formulations for injection may be presented in unit dosage form, for example, in ampoules or in multi-dose containers, with an added preservative. The compositions may take such forms as suspensions, solutions or emulsions in oily or aqueous vehicles, and may contain agents for a pharmaceutical formulation, such as suspending, stabilizing and/or dispersing agents. Alternatively, the rAAV vectors and/or rAAV genome as disclosed herein can be in powder form (e.g., lyophilized) for constitution with a suitable vehicle, for example, sterile pyrogen-free water, before use.

In particular embodiments, more than one administration (e.g., two, three, four, five, six, seven, eight, nine, 10, etc., or more administrations) may be employed to achieve the desired level of FVIII expression over a period of various intervals, e.g., hourly, daily, weekly, monthly, yearly, etc. Dosing can be single dosage or cumulative (serial dosing), and can be readily determined by one skilled in the art. As disclosed herein, it is envisioned that treatment of a subject (e.g., for Hemophilia A) according to the methods as disclosed herein comprises a one-time administration of an effective dose of a pharmaceutical composition comprising a AAV vector comprising the heterologous codon-optimized nucleic acid encoding a human FVIII polypeptide.

However, in alternative embodiments, treatment of a subject with Hemophilia A may comprise multiple administrations of a pharmaceutical composition comprising a AAV vector comprising the heterologous codon-optimized nucleic acid encoding a human FVIII polypeptide, described herein, wherein the multiple administrations can be carried out over a range of time periods, such as, e.g., once yearly, or every 6-months, or about every 2-years, or about every 3-years, or about every 4 years, or about every 5-years or longer than 5-year intervals. The timing of administration can vary from individual to individual, depending upon such factors as the severity of an individual's symptoms. For example, in some embodiments, an effective dose of a AAV vector as disclosed herein can be administered to an individual once every year, or once every two years, or every six months for an indefinite period of time, or until the individual no longer requires any additional anti-Hemophilia A therapy. A person of ordinary skill in the art will recognize that the condition of the individual can be monitored throughout the course of treatment and that the effective amount of a AAV vector as disclosed herein that is administered can be adjusted accordingly.

Injectables comprising a AAV vector comprising the heterologous codon-optimized nucleic acid encoding a human FVIII polypeptide, as disclosed herein, can be prepared in conventional forms, either as liquid solutions or suspensions, solid forms suitable for solution or suspension in liquid prior to injection, or as emulsions. Alternatively, one may administer the AAV vector in a local rather than systemic manner, for example, in a depot or sustained-release formulation. Further, the virus vector and/or virus capsid can be delivered adhered to a surgically implantable matrix (e.g., as described in U.S. Patent Publication No. US-2004-0013645-A1). In some embodiments, the AAV vector can be administered to the lungs of a subject by any suitable means, optionally by administering an aerosol suspension of respirable particles comprised of the virus vectors and/or virus capsids, which the subject inhales. The respirable particles can be liquid or solid. Aerosols of liquid particles comprising the virus vectors and/or virus capsids may be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the virus vectors and/or capsids may likewise be produced with any solid particulate medicament aerosol generator, by techniques known in the pharmaceutical art.

In some embodiments, a AAV vector comprising the heterologous codon-optimized nucleic acid encoding a human FVIII polypeptide, as disclosed herein can be formulated in a solvent, emulsion or other diluent in an amount sufficient to dissolve an rAAV vector. In other aspects of this embodiment, the rAAV vectors and/or rAAV genome encoding FVIII polypeptide as disclosed herein can herein may be formulated in a solvent, emulsion or a diluent in an amount of, e.g., less than about 90% (v/v), less than about 80% (v/v), less than about 70% (v/v), less than about 65% (v/v), less than about 60% (v/v), less than about 55% (v/v), less than about 50% (v/v), less than about 45% (v/v), less than about 40% (v/v), less than about 35% (v/v), less than about 30% (v/v), less than about 25% (v/v), less than about 20% (v/v), less than about 15% (v/v), less than about 10% (v/v), less than about 5% (v/v), or less than about 1% (v/v). In other aspects, the rAAV vectors and/or rAAV genome encoding a FVIII polypeptide as disclosed herein can disclosed herein may comprise a solvent, emulsion or other diluent in an amount in a range of, e.g., about 1% (v/v) to 90% (v/v), about 1% (v/v) to 70% (v/v), about 1% (v/v) to 60% (v/v), about 1% (v/v) to 50% (v/v), about 1% (v/v) to 40% (v/v), about 1% (v/v) to 30% (v/v), about 1% (v/v) to 20% (v/v), about 1% (v/v) to 10% (v/v), about 2% (v/v) to 50% (v/v), about 2% (v/v) to 40% (v/v), about 2% (v/v) to 30% (v/v), about 2% (v/v) to 20% (v/v), about 2% (v/v) to 10% (v/v), about 4% (v/v) to 50% (v/v), about 4% (v/v) to 40% (v/v), about 4% (v/v) to 30% (v/v), about 4% (v/v) to 20% (v/v), about 4% (v/v) to 10% (v/v), about 6% (v/v) to 50% (v/v), about 6% (v/v) to 40% (v/v), about 6% (v/v) to 30% (v/v), about 6% (v/v) to 20% (v/v), about 6% (v/v) to 10% (v/v), about 8% (v/v) to 50% (v/v), about 8% (v/v) to 40% (v/v), about 8% (v/v) to 30% (v/v), about 8% (v/v) to 20% (v/v), about 8% (v/v) to 15% (v/v), or about 8% (v/v) to 12% (v/v).

In any embodiment of the methods as disclosed herein, a AAV vector comprising the heterologous codon-optimized nucleic acid encoding a human FVIII polypeptide, as disclosed herein, can be an AAV of any serotype, including but not limited to encapsulated by any AAV8 capsid, or any AAV3b capsid selected from: AAV3b capsid (SEQ ID NO: 452); AAV3b265D capsid (SEQ ID NO: 454), AAV3b ST (S663V+T492V) capsid (SEQ ID NO: 456), AAV3b265D549A capsid (SEQ ID NO: 458); AAV3b549A capsid (SEQ ID NO: 460); AAV3bQ263Y capsid (SEQ ID NO: 462) or AAV3bSASTG capsid (i.e., a AAV3b capsid comprising Q263A/T265 mutations), and those disclosed in Table 2 above.

To facilitate delivery of a AAV vector comprising the heterologous codon-optimized nucleic acid encoding a human FVIII polypeptide, as disclosed herein, it can be mixed with a carrier or excipient. Carriers and excipients that might be used include saline (especially sterilized, pyrogen-free saline) saline buffers (for example, citrate buffer, phosphate buffer, acetate buffer, and bicarbonate buffer), amino acids, urea, alcohols, ascorbic acid, phospholipids, proteins (for example, serum albumin), EDTA, sodium chloride, liposomes, mannitol, sorbitol, and glycerol. USP grade carriers and excipients are particularly useful for delivery of virions to human subjects.

In addition to the formulations described previously, a AAV vector comprising the heterologous codon-optimized nucleic acid encoding a human FVIII polypeptide, as disclosed herein can also be formulated as a depot preparation. Such long-acting formulations may be administered by implantation (for example subcutaneously or intramuscularly) or by IM injection. Thus, for example, a rAAV vector and/or rAAV genome as disclosed herein may be formulated with suitable polymeric or hydrophobic materials (for example as an emulsion in an acceptable oil) or ion exchange resins, or as sparingly soluble derivatives.

In any embodiment of the methods as disclosed herein, the method is directed to treating a disease or disorder, e.g., Hemophilia A, that results from a deficiency of FVIII in a subject, wherein a AAV vector comprising the heterologous codon-optimized nucleic acid encoding a human FVIII polypeptide, as disclosed herein is administered to a patient suffering from Hemophilia A, and following administration, FVIII polypeptide is secreted from cells in the liver and there is uptake of the secreted FVIII polypeptide. In some embodiments, the AAV vector is encapsulated in a capsid, e.g., encapsulated by any AAV3b capsid selected from: AAV3b capsid (SEQ ID NO: 452); AAV3b265D capsid (SEQ ID NO: 454), AAV3b ST (S663V+T492V) capsid (SEQ ID NO: 456), AAV3b265D549A capsid (SEQ ID NO: 458); AAV3b549A capsid (SEQ ID NO: 460); AAV3bQ263Y capsid (SEQ ID NO: 462) or AAV3bSASTG capsid (i.e., a AAV3b capsid comprising Q263A/T265 mutations).

In a particular embodiment, at least about 1.6×10¹² to about 4.0×10¹² vg/kg will be administered per dose in a pharmaceutically acceptable carrier. In some embodiments, at least about 1.0×10¹⁰ to about 1.0×10¹³ vg/kg will be administered per dose in a pharmaceutically acceptable carrier. In a further embodiment, dosages of the virus vector and/or capsid to be administered to a subject depend upon the mode of administration, the individual subject's condition, age and gender, and the particular virus vector or capsid, the nucleic acid encoding FVIII polypeptide to be delivered, and the like, and can be determined in a routine manner.

Exemplary doses for achieving therapeutic effects are titers of at least about 1.5×10¹⁰ vg/kg, at least about 1.5×10¹¹ vg/kg, or at least about 1.5×10¹² vg/kg, or at least about 4.0×10¹² vg/kg. It is encompassed that the dose for achieving therapeutic effects as disclosed herein may also be determined by the strength of the promoter operatively linked to the nucleic acid encoding the FVIII polypeptide. In contrast, the dose of the AAV herein can be lower than about 1.6×10¹² when the promoter, for example, is stronger than the liver specific promoter (SEQ ID NO: 97), however, the dose of AAV should be titrated and determined based on the level of FVIII polypeptide expressed in the cell, as determined by transduction efficiency of the AAV capsid and the LSP, and the ability of the cell to secrete the expressed FVIII polypeptide in order to avoid FVIII polypeptide accumulation in the transfected cell and any associated cell toxicity.

In another aspect, disclosed herein is a method of treating Hemophilia A by administering a codon-optimized nucleic acid encoding a human FVIII polypeptide in expressible form to a cell, of a patient, comprising contacting the cell with a rAAV vector and/or rAAV genome as disclosed herein, under conditions for the nucleic acid to be introduced into the cell and expressed to produce the FVIII polypeptide. In some embodiments, the cell is a cell in vivo. In some embodiments, the cell is a mammalian cell in vivo.

In any embodiment of the methods as disclosed herein, a AAV vector encoding a FVIII polypeptide as disclosed herein is useful in methods to decrease symptoms a mammal caused by Hemophilia A and/or insufficient FVIII levels.

In an embodiment, a rAAV capsid of the rAAV virion used to treat Hemophilia A is any of those listed in Table 1 as disclosed in International Applications WO2020/102645, and WO2020/102667, each of which are incorporated herein in their entirety, and includes any of AAV8 or AAV3, or AAV3b (including but not limited to AAV3b serotypes AAV3b265D, AAV3b265D549A, AAV3b549A, AAV3bQ263Y, AAV3bSASTG (i.e., a AAV3b capsid comprising Q263A/T265 mutations) serotypes). In some embodiments, treatment with the rAAV virion comprising the codon-optimized nucleic acid encoding the human FVIII, as disclosed herein, is capable of reducing any one or more of Hemophilia-caused bleeding, frequency or severity of acute bleeding episodes, blood clotting time, activated thromboplastin time assay, in a patient suffering from Hemophilia A by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment. In other aspects of this embodiment, an AAV containing encoding FVIII of any serotype is capable of reducing any one or more of the systems of Hemophilia-caused bleeding, frequency or severity of acute bleeding episodes, blood clotting time, activated thromboplastin time assay, in a patient suffering from Hemophilia A by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to a patient not receiving the same treatment.

In any embodiment of the methods and compositions as disclosed herein, at least one symptom associated with Hemophilia A, or at least one adverse side effect associated with Hemophilia A are reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, and the severity of at least one symptom associated with Hemophilia A, or at least one adverse side effect is reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In another embodiment, at least one symptom associated with Hemophilia A, or at least one adverse side effect associated with Hemophilia A is reduced by about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

### Methods of Administration

Accordingly, in one embodiment, the technology relates to a method of treating Hemophilia A in a subject, comprising administering to the subject a pharmaceutical composition comprising a recombinant adeno-associated virus (AAV) vector comprising in its genome, a heterologous codon-optimized nucleic acid sequence encoding an FVIII polypeptide in expressible form wherein the heterologous nucleic acid is operatively linked to a promoter (e.g., a liver specific promoter), in the absence or presence of administration of an additional anti-Hemophilia A therapy. In some embodiments, the dosage of the recombinant AAV ranges from 1.0E⁹ vg/kg to 5.0E¹² vg/kg, and in some embodiments, the FVIII is expressed to a level that the subject obtains a blood serum level of FVIII expressed by the AAV at a pharmaceutical activity range from at least 25% to about 150% of normal, or at least 50% to about 150% of normal, e.g., at least within two weeks of administration.

In some embodiments, the FVIII is expressed to a level that the subject obtains a blood serum level of FVIII expressed by the AAV at a pharmaceutical activity range from at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 101%, 102%, 103%, 104%, 105%, 106%, 107%, 108%, 109%, 110%, 111%, 112%, 113%, 114%, 115%, 116%, 117%, 118%, 119%, 120%, 121%, 122%, 123%, 124%, 125%, 126%, 127%, 128%, 129%, 130%, 131%, 132%, 133%, 134%, 135%, 136%, 137%, 138%, 139%, 140%, 141%, 142%, 143%, 144%, 145%, 146%, 147%, 148%, or 149% of normal, e.g., at least within two weeks of administration. In one embodiment, the FVIII is expressed to a level that the subject obtains a blood serum level of FVIII expressed by the AAV at a pharmaceutical activity no more than 150%, e.g., at least within two weeks of administration.

In some embodiments, the FVIII is expressed to a level that the subject obtains a blood serum level of FVIII expressed by the AAV at a pharmaceutical activity range from at least 15-140%, 55-150%, 60-150%, 65-150%, 70-150%, 75-150%, 80-150%, 85-150%, 90-150%, 95-150%, 100-150%, 105-150%, 110-150%, 115-150%, 120-150%, 125-150%, 130-150%, 135-150%, 140-150%, 145-150%, 50-145%, 50-140%, 50-135%, 50-130%, 50-125%, 50-120%, 50-115%, 50-110%, 50-105%, 50-100%, 50-95%, 50-90%, 50-85%, 50-80%, 50-75%, 50-70%, 50-65%, 50-60%, 50-55%, 60-140%, 70-130%, 80-120%, 90-110%, 100-110% of normal activity, e.g., at least within two weeks of administration.

In some embodiments, the FVIII is expressed to a level that the subject obtains a blood serum level of FVIII expressed by the AAV at a pharmaceutical activity range from at least 25 IU/dL for normal activity, or at least 50 IU/dL to about 150 IU/dL for normal activity, e.g., at least within two weeks of administration.

In some embodiments, the FVIII is expressed to a level that the subject obtains a blood serum level of FVIII expressed by the AAV at a pharmaceutical activity range from at least 5 IU/dL, 6 IU/dL, 7 IU/dL, 8 IU/dL, 9 IU/dL, 10 IU/dL, 11 IU/dL, 12 IU/dL, 13 IU/dL, 14 IU/dL, 15 IU/dL, 16 IU/dL, 17 IU/dL, 18 IU/dL, 19 IU/dL, 20 IU/dL, 21 IU/dL, 22 IU/dL, 23 IU/dL, 24 IU/dL, 25 IU/dL, 26 IU/dL, 27 IU/dL, 28 IU/dL, 29 IU/dL, 30 IU/dL, 31 IU/dL, 32 IU/dL, 33 IU/dL, 34 IU/dL, 35 IU/dL, 36 IU/dL, 37 IU/dL, 38 IU/dL, 39 IU/dL, 40 IU/dL, 41 IU/dL, 42 IU/dL, 43 IU/dL, 44 IU/dL, 45 IU/dL, 46 IU/dL, 47 IU/dL, 48 IU/dL, 49 IU/dL, 50 IU/dL, 51 IU/dL, 52 IU/dL, 53 IU/dL, 54 IU/dL, 55 IU/dL, 56 IU/dL, 57 IU/dL, 58 IU/dL, 59 IU/dL, 60 IU/dL, 61 IU/dL, 62 IU/dL, 63 IU/dL, 64 IU/dL, 65 IU/dL, 66 IU/dL, 67 IU/dL, 68 IU/dL, 69 IU/dL, 70 IU/dL, 71 IU/dL, 72 IU/dL, 73 IU/dL, 74 IU/dL, 75 IU/dL, 76 IU/dL, 77 IU/dL, 78 IU/dL, 79 IU/dL, 80 IU/dL, 81 IU/dL, 82 IU/dL, 83 IU/dL, 84 IU/dL, 85 IU/dL, 86 IU/dL, 87 IU/dL, 88 IU/dL, 89 IU/dL, 90 IU/dL, 91 IU/dL, 92 IU/dL, 93 IU/dL, 94 IU/dL, 95 IU/dL, 96 IU/dL, 97 IU/dL, 98 IU/dL, 99 IU/dL, 100 IU/dL, 101 IU/dL, 102 IU/dL, 103 IU/dL, 104 IU/dL, 105 IU/dL, 106 IU/dL, 107 IU/dL, 108 IU/dL, 109 IU/dL, 110 IU/dL, 111 IU/dL, 112 IU/dL, 113 IU/dL, 114 IU/dL, 115 IU/dL, 116 IU/dL, 117 IU/dL, 118 IU/dL, 119 IU/dL, 120 IU/dL, 121 IU/dL, 122 IU/dL, 123 IU/dL, 124 IU/dL, 125 IU/dL, 126 IU/dL, 127 IU/dL, 128 IU/dL, 129 IU/dL, 130 IU/dL, 131 IU/dL, 132 IU/dL, 133 IU/dL, 134 IU/dL, 135 IU/dL, 136 IU/dL, 137 IU/dL, 138 IU/dL, 139 IU/dL, 140 IU/dL, 141 IU/dL, 142 IU/dL, 143 IU/dL, 144 IU/dL, 145 IU/dL, 146 IU/dL, 147 IU/dL, 148 IU/dL, or 149 IU/dL for normal activity, e.g., at least within two weeks of administration. In one embodiment, the FVIII is expressed to a level that the subject obtains a blood serum level of FVIII expressed by the AAV at a pharmaceutical activity no more than 150 IU/dL, e.g., at least within two weeks of administration.

In some embodiments, the FVIII is expressed to a level that the subject obtains a blood serum level of FVIII expressed by the AAV at a pharmaceutical activity range from at least 15-140 IU/dL, 55-150 IU/dL, 60-150 IU/dL, 65-150 IU/dL, 70-150 IU/dL, 75-150 IU/dL, 80-150 IU/dL, 85-150 IU/dL, 90-150 IU/dL, 95-150 IU/dL, 100-150 IU/dL, 105-150 IU/dL, 110-150 IU/dL, 115-150 IU/dL, 120-150 IU/dL, 125-150 IU/dL, 130-150 IU/dL, 135-150 IU/dL, 140-150 IU/dL, 145-150 IU/dL, 50-145 IU/dL, 50-140 IU/dL, 50-135 IU/dL, 50-130 IU/dL, 50-125 IU/dL, 50-120 IU/dL, 50-115 IU/dL, 50-110 IU/dL, 50-105 IU/dL, 50-100 IU/dL, 50-95 IU/dL, 50-90 IU/dL, 50-85 IU/dL, 50-80 IU/dL, 50-75 IU/dL, 50-70 IU/dL, 50-65 IU/dL, 50-60 IU/dL, 50-55 IU/dL, 60-140 IU/dL, 70-130 IU/dL, 80-120 IU/dL, 90-110 IU/dL, or 100-110 IU/dL for normal activity, e.g., at least within two weeks of administration.

In some embodiments, the dosage of the AAV ranges from 1.0E⁹ vg/kg to 5.0E¹⁷ vg/kg, and in some embodiments, is no more than 4.0E¹³ vg/kg, and in some embodiments, the FVIII is expressed to a level that the subject obtains a blood serum level of FVIII expressed by the AAV at a pharmaceutical activity range from 189 to ≤2,260 nmol/mL/hr of at least within two weeks of administration. In some embodiments, the dosage of the AAV is no more than 4.0E¹³ vg/kg, and in some embodiments, the FVIII is expressed to a level that the subject obtains a blood serum level of FVIII expressed by the AAV at a pharmaceutical activity range from 189 to ≤2,260 nmol/mL/hr of at least within two weeks of administration.

In some embodiments, the dosage of AAV containing the codon-optimized nucleic acid encoding the FVIII polypeptide is no more than 5.0E¹⁷ vg/kg. In some embodiments, the dosages range from 1.0E⁹ vg/kg to 5.0E¹⁷ vg/kg.

In particular, the technology described herein relates to the discovery that a single infusion of a rAAV vector containing the codon-optimized nucleic acid encoding the FVIII polypeptide can be a stand-alone therapeutic. In one embodiment, a one-time administration of the AAV leads to long-term transduction of the FVIII polypeptide into hepatocytes and continuous constitutive expression of FVIII polypeptide in the systemic circulation.

In one embodiment, described herein is a method of treating Hemophilia A in a subject in need thereof by administering the subject a composition comprising a AAV vector containing the codon-optimized nucleic acid encoding the FVIII polypeptide, where the subject is not being concurrently administered any additional anti-Hemophilia A therapies. In some embodiments, the technology relates to a method of administering the AAV where the subject has not been administrered any additional anti-Hemophilia A therapies for an extended period of time, e.g., at least 3 months, at least 4 months, at least 5 months, at least 1 year, at least 1½ years and points in between 6 months or longer. In some embodiments, the subject has not been administered an additional anti-Hemophilia A therapy on the day of, or shortly before administration of the AAV.

In one embodiment, described herein is a method of treating Hemophilia A in a subject in need thereof by administering the subject a composition comprising a AAV vector comprising the codon-optimized nucleic acid encoding the FVIII polypeptide, where the subject is concurrently administered at least one additional anti-Hemophilia A therapies. In some embodiments, the technology relates to a method of administering the AAV where the subject has been administrered at least one additional anti-Hemophilia A therapies for an extended period of time, e.g., at least 3 months, at least 4 months, at least 5 months, at least 1 year, at least 1½ years and points in between 6 months or longer. In some embodiments, the subject has been administered at least one additional anti-Hemophilia A therapy on the day of, or shortly before administration of the AAV.

Subjects administered a AAV encoding the codon-optimized nucleic acid encoding the FVIII polypeptide according to the methods and dose ranges as disclosed herein, can exhibit a minimal immune response to the FVIII protein expressed by the AAV. According, in some embodiments, there is minimal, or no need for immune modulation or administration of immune suppressants at the time of, or before, or after the administration of the AAV to the subject, and therefore normal immune suppressants protocols which are typically administered when a subject is administered a viral vector, or undergoing gene therapy are not required.

In certain aspects, the AAV that comprise a nucleotide sequence containing inverted terminal repeats (ITRs), a promoter, a heterologous gene, a poly-A tail and potentially other regulator elements for use to treat a at least one, wherein the heterologous gene is the codon-optimized nucleic acid encoding the FVIII polypeptide, and wherein the vector, e.g., rAAV can be administered to a patient in a therapeutically effective dose that is delivered to the appropriate tissue and/or organ for expression of the FVIII polypeptide and treatment of the disease, e.g., at least one.

In some embodiments, the method to treat at least one with rAAV comprising the heterologous codon-optimized nucleic acid encoding a human FVIII polypeptide, as disclosed herein comprises administration of a therapeutically effective amount of a rAAV disclosed herein to result in a serum level of the expressed FVIII polypeptide within a pharmacological activity range of between 189 to 410 nmol/mL/hr, or 410 to ≤2,260 nmol/mL/hr.

### AAV-FVIII Dosages

In some embodiments, the methods disclosed herein relate to human subjects can be administered a rAAV containing the codon-optimized nucleic acid encoding the FVIII polypeptide as disclosed herein at a dose in the range of 1.0E⁹ vg/kg to 5.0E¹⁷ vg/kg. In some embodiments, there can be a therapeutic correction of disease pathophysiology with administration of the rAAV, as disclosed herein and also protection against immune response to the expressed FVIII polypeptide e.g, as measured by the antibodies against the expressed hFVIII polypeptide.

In some embodiments the dose of the a rAAV vector or rAAV genome to be administered to the subject according to the method to treat Hemophilia A as disclosed herein depends upon the mode of administration, the promoter used, the severity of the disease or other condition to be treated and/or prevented, the individual subject's condition, the particular virus vector or capsid, the promoter being used and the nucleic acid to be delivered, including but not limited to, nucleic acid encoding the signal peptide attached to the 5′ of the nucleic acid encoding expressible FVIII polypeptide, and the like, and can be determined in a routine manner.

Native FVIII levels in normal humans range from about 150-200 ng/ml plasma, but may be less (e.g., range of about 100-150 ng/ml) or greater (e.g., range of about 200-300 ng/ml) and still considered normal due to functioning clotting as determined, for example, by an activated partial thromboplastin time (aPTT) one-stage clotting assay. Thus, a therapeutic effect can be achieved by expression of the FVIII polypeptide such that the total amount of FVIII polypeptide in the subject/human is greater than 1% of the native FVIII present in normal subjects/humans, e.g., 1% of 100-300 ng/ml.

In some embodiments, the dose of the rAAV vector comprising the codon-optimized nucleic acid encoding a human FVIII polypeptide, is a therapeutically effective amount to increase the blood or plasma level of FVIII polypeptide levels in the subject to therapeutic levels. In some embodiments, the dose of the rAAV vector is a therapeutically effective amount to increase FVIII polypeptide blood or plasma content in the subject to within 40%, or within 30%, or within 20%, or within 10%, or within 9%, 8%, 7%, 6%, 5%, 4%, 3%, 2% or 1% of the native active form of FVIII blood or plasma content, where the blood or plasma content of the native active form of FVIII naturally present in a subject without Hemophilia A is used as a reference level. In some embodiments, the dose of the rAAV vector is a therapeutically effective amount to increase blood or plasma FVIII polypeptide content in the subject more than 2-fold, or 3-fold, or 4-fold, or 5-fold, or 6-fold, or 7-fold, or 8-fold, or 9-fold, or 10-fold, or more than 10-fold of the level of FVIII blood or plasma content in the subject with Hemophilia A. In some embodiments, the dose of the rAAV vector is a therapeutically effective amount to increase blood or plasma FVIII polypeptide content in the subject to about 50%, or, about 40%, or about 30%, or about 20%, or about 10%, or about 9%, about 8%, about 7%, about 6%, about 5%, about 4%, about 3%, about 2% or about 1% of the level of FVIII blood or plasma content in the subject with Hemophilia A. In some embodiments, the FVIII polypeptide activity in plasma is at least 1.5 fold, at least 2 fold, at least 3 fold, at least 5 fold, at least 8 fold, or at least 10 fold than the level prior to AAV administration.

In some embodiments, the dose of the rAAV vector comprising the codon-optimized nucleic acid encoding a human FVIII polypeptide is a therapeutically effective amount of rAAV vector to exhibit an improvement in the therapeutic index of 3- to 5-fold. In some embodiments, the dose of the rAAV vector is a therapeutically effective amount to result in the subject having clinically stable levels of hFVIII polypeptide at 10-weeks, or at least 20 weeks, or 30 weeks post rAAV administration.

In an embodiment, as used herein, without limitation, the term “effective amount” is synonymous with “therapeutically effective amount”, “effective dose”, or “therapeutically effective dose.” In an embodiment, the effectiveness of a therapeutic compound disclosed herein to treat Hemophilia A can be determined, without limitation, by observing an improvement in an individual based upon one or more clinical symptoms, and/or physiological indicators associated with Hemophilia A. In an embodiment, an improvement in the symptoms associated with Hemophilia A can be indicated by a reduced need for a concurrent therapy for example, less frequent or reduced dose or elimination of administration of a recombinant clotting factor protein to supplement for the deficient or defective (abnormal or mutant) endogenous clotting factor in the subject.

For hemophilia therapy, efficacy of the treatment can, for example, be measured by assessing the hemophilia-caused bleeding in the subject. An effective amount would be an amount that reduces frequency or severity of acute bleeding episodes in a subject, for example, or an amount that reduces clotting time as measured by a clotting assay, for example. In vitro tests such as, but not limited to the in vitro activated partial thromboplastin time assay (APPT), test factor IX chromogenic activity assays, blood clotting times, factor IX or human factor VIII-specific ELISAs are also available. Other tests for assessing the efficacy of the treatment known in the art can also be used.

In some embodiments, exemplary doses for achieving therapeutic effects of a rAAV comprising the codon-optimized nucleic acid encoding a human FVIII polypeptide as disclosed herein is within the range of 1.0E⁹ vg/kg to 5.0E¹⁷ vg/kg. In some embodiments, the dose administerered to a subject is at least about 1.0E⁹ vg/kg, at least about 1.0E¹⁰ vg/kg, at least about 1.0E11 vg/kg, at least about 1.0E12 vg/kg, about 1.1E12 vg/kg, about 1.2E12 vg/kg, about 1.3E12 vg/kg, about 1.4E12 vg/kg, about 1.5E12 vg/kg, about 1.6E12 vg/kg, about 1.7E12 vg/kg, about 1.8E12 vg/kg, about 1.9E12 vg/kg, about 2.0E12 vg/kg, about 3.0E12 vg/kg, about 4.0E12 vg/kg, about 5.0E12 vg/kg, about 6.0E12 vg/kg, about 7.0E12 vg/kg, about 8.0E12 vg/kg, about 9.0E12 vg/kg, about 1.0E13 vg/kg, about 1.2E13 vg/kg, about 1.2E13 vg/kg, about 1.2E13 vg/kg, about 1.3E13 vg/kg, about 1.4E13 vg/kg, about 1.5E13 vg/kg, about 1.6E13 vg/kg, about 1.7E13 vg/kg, about 1.8E13 vg/kg, about 1.9E13 vg/kg, about 2.0E13 vg/kg, about 3.0E13 vg/kg, about 4.0E13 vg/kg, about 5.0E13 vg/kg.

In preferred embodiments, exemplary doses for achieving therapeutic effects according to the methods as disclosed herein are titers of at between 1.2E12 and 4.0E12 vg/kg, for example, least about 1.0E12 vg/kg, about 1.1E12 vg/kg, about 1.2E12 vg/kg, about 1.3E12 vg/kg, about 1.4E12 vg/kg, about 1.5E12 vg/kg, about 1.6E12 vg/kg, about 1.7E12 vg/kg, about 1.8E12 vg/kg, about 1.9E12 vg/kg, about 2.0E12 vg/kg, about 2.1E12 vg/kg, about 2.2E12 vg/kg, about 2.3E12 vg/kg, about 2.4E12 vg/kg, about 2.5E12 vg/kg, about 2.6E12 vg/kg, about 2.7E12 vg/kg, about 2.8E12 vg/kg, about 2.9E12 vg/kg, about 3.0E12 vg/kg, about 3.1E12 vg/kg, about 3.2E12 vg/kg, about 3.3E12 vg/kg, about 3.4E12 vg/kg, about 3.5E12 vg/kg, about 3.6E12 vg/kg, about 3.7E12 vg/kg, about 3.8E12 vg/kg, about 3.9E12 vg/kg, about 4.0E12 vg/kg.

In some embodiments, a rAAV vector comprising the codon-optimized nucleic acid encoding a human FVIII polypeptide as disclosed herein useful for the methods to treat Hemophilia A, exemplary doses for achieving therapeutic effects are titers of at least about 1.0E12 to 4.0E12 vg/kg, or about 1.2E12 to 3.0E12 vg/kg, or about 1.2E12 to 2.5E12 vg/kg, or about 2.5E12 to 4.0E12 vg/kg.

In some embodiments, the dosage may be modified by a person of ordinary skill in the art, e.g., the dose administered can be lower than 1.0E12 vg/kg, or lower than about 5.0E11 vg/kg where a stronger promoter is operatively linked to the nucleic acid encoding FVIII polypeptide. In contrast, in alternative embodiments, the dosage may be modified by a person of ordinary skill in the art, e.g., the dose of the rAAV vector administered can be higher than about 1.6E12 vg/kg, or higher than about 5.0E12 vg/kg when a weaker promoter used in the vector is operatively linked to the nucleic acid encoding the FVIII polypeptide. Exemplary doses for achieving therapeutic effects are titers of at least about 1.0E⁵, 1.0E⁶, 1.0E⁷, 1.0E⁸, 1.0E⁹, 1.0E¹⁰, 1.0E¹¹, 1.0E¹² vg/kg, optionally about 1.0E¹⁰ to about 1.0E¹² transducing units (vg/kg), and optionally does not exceed about 4.0E¹² vg/kg or optionally is about 3.0E¹² transducing units (vg/kg).

In a further embodiment, administration of rAAV vector or rAAV genome according to the methods as disclosed herein to treat a subject with Hemophilia A can result in production of a FVIII polypeptide with a circulatory half-life of 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 1 week, 2 weeks, 3 weeks, 4 weeks, one month, two months, three months, four months or more.

In some embodiments, the methods for treatment of Hemophilia A as disclosed herein relate to a single dose of a rAAV comprising the codon-optimized nucleic acid encoding a human FVIII polypeptide is used to treat a subject in a single administration. However, in some embodiments, the dose of rAAV to be administered can be given to the subject in multiple administrations, e.g., a dose of rAAV can be divided into sub-doses and administered in multiple administrations.

In some embodiments, it is envisioned that the methods for treatment of Hemophilia A as disclosed herein can comprise multiple administrations of a single dose of a rAAV comprising the codon-optimized nucleic acid encoding a human FVIII polypeptide, that is, the subject can be treated with a booster administration (i.e., a second, third, fourth, etc.) of the rAAV after a defined period of time after the initial or first administration. The dose of a booster administration (i.e., 2^{nd}, 3^{rd}, 4^{th}, or 5^{th} etc.) can be the same dose (amount) of rAAV administered in the first administration, or can be a higher dose, or a lower dose, depending on the factors above, including, but not limited to, a therapeutically effective dose to achieve any one or more of (i) serum FVIII polypeptide levels indicating steady state of FVIII polypeptide expression and (ii) substantial reduction in one or more Hemophilia A symptoms, including, without limitation, Hemophilia-caused bleeding, frequency or severity of acute bleeding episodes, blood clotting time, activated thromboplastin time assay, to within clinically stable levels. As disclosed herein, a steady state of FVIII polypeptide expression by the rAAV as disclosed herein is a serum level of FVIII polypeptide tha provides a therapeutic effect. Stability of one or more symptoms of Hemophilia A can be determined by the clinical stability parameters as disclosed herein.

In an embodiment, the time period of between administration of a first dose, and a subsequent dose (i.e., a booster dose) of a rAAV vector according to the methods for treatment of Hemophilia A as disclosed herein is selected from any of the following: about 4 months, about 6 months, about 7 months, about 8 months, about 9 months, about 12 months, about 18 months, about 24 months, or about 3 years, about 4 years, about 5 years, or more than 5 years.

### Immune Suppression

In another aspect, the technology relates to methods to treat Hemophilia A by administering a rAAV vector containing a codon-optimised nucleic acid encoding a human FVIII polypeptide as disclosed herein, where the administration of a composition comprising a AAV vector is administered to the subject without ongoing immune suppression. That is, in some embodiments, immune suppression is not administered to the subject long term.

In some embodiments, an immune suppressant or immune modulator is administered to the subject intermittently, or for a transient period, e.g., as an immune prophylaxis to the subject to prevent or reduce any immune response to the administered AAV vector, therefore allowing, if necessary, a subsequent or booster administration of the AAV vector according to the methods as disclosed herein.

In some embodiments, an immune modulator is administered for an initial period at, or around the time the rAAV vector containing a codon-optimised nucleic acid encoding a human FVIII polypeptide as disclosed herein, is administered to the subject. For example, an immune modulator is administered starting at about 24 hrs before the rAAV vector is administered to the subject. In some embodiments, an immune modulator is administered starting at about 24 hrs before the rAAV administration and is administered for at least 1 day, or at least 2 days, or at least 3 days or at least 4 days, or at least 5 days, or at least 6 days, or for about 1 week, or for longer than 1 week after administration of the rAAV vector. In some embodiments, an immune modulator is administered starting at, or about 24 hrs before rAAV administration and is administered for no more than 1 day, or 2 days, 3 days, or 4 days, or 5 days, or 6 days, or for 1 week, or for 2 weeks, or for 3 weeks or for 1 month after administration of the rAAV.

In some embodiments, an immune modulator is administered to the subject at tapering lower doses, e.g., at a first dose for a first period of time, at a second lower dose for a second period of time, and third dose that is lower than the second dose—for a third period of time, and so forth until no immune response to the AAV or the FVIII polypeptide is produced. For example, in some embodiments, the first dose of an immune modulator is started at, or about 24 hrs before rAAV administration and is administered for at least 1 day, or at least 2 days, or at least 3 days or at least 4 days, or at least 5 days, or at least 6 days, or for about 1 week, or about 2 weeks, or about 3 weeks, or about 4 weeks, after which the immune modulator is reduced to a third dose (which is lower than the second dose) for a third period of time (e.g., for at least 1 day, or at least 2 days, or at least 3 days or at least 4 days, or at least 5 days, or at least 6 days, or for about 1 week).

For exemplary purposes only, in some embodiments, the methods to treat Hemophilia A as disclosed herein comprise administering prednisone as an immune suppressant, i.e., immune prophylaxis, at a first dose of 60 milligrams (given orally) starting 24 hours prior to rAAV vector administration. In some embodiments, prednisone is continued at 60 mg/day po through the completion of week four after vector administration, after which, at the beginning of week 5 the prednisone dose is tapered to a second dose level of 55 mg/day po and maintained for 7 days. In some embodiments, at the beginning of week 6 the dose is tapered to a third dose level of 50 mg/day po and maintained for 7 days etc., so that the dose of the immune suppressant (i.e., prednisone) is tapered on a weekly basis by 5 mg/day, after an initial immune suppressant dose for 4 weeks.

The use of prednisone is exemplified herein as an immune suppressant for immune prophylaxis according to the methods as disclosed herein. However, it is envisioned that prednisone can be readily substituted with a different immune modulator and administration regimen known by a person of ordinary skill in the art.

In some embodiments, normal immune prophylaxis for preventing immune reactivity to the rAAV or the expressed FVIII polypeptide is stopped, or withdrawn on day 1, or shortly before or after administration of the rAAV according to the methods as disclosed herein.

### Immune Modulation and Immunosuppression:

As disclosed herein, in some embodiments, the methods to treat Hemophilia A by administering a rAAV containing a codon-optimized nucleic acid encoding a human FVIII polypeptide as disclosed herein, to the subject without ongoing immune suppression. That is, in some embodiments, immune suppression is not administered to the subject long term, and is only administered for a short and pre-defined period, including an initial period (with an initial dose) and a tapering period (with incremental tapering doses) after the administration of the AAV vector to the subject. Accordingly, in some embodiments, the immune suppression is administered for between 4 weeks to up to about 15 weeks after the administration of the AAV vector to the subject, and can be administered in an initial and tapering doses as disclosed herein.

Accordingly, in some embodiments, the methods and compositions using the AAV vectors and AAV genomes as described herein, for treating Hemophilia A, further comprises administering an immune modulator for an initial period followed by a tapering period. In some embodiments, the immune modulator can be administered at the time of rAAV vector administration, before rAAV vector administration or, after the rAAV vector administration.

In any embodiment of the methods and compositions as disclosed herein, a subject being administered a rAAV vector or rAAV genome as disclosed herein is also administered an immunosuppressive agent. Various methods are known to result in the immunosuppression of an immune response of a patient being administered AAV. Methods known in the art include administering to the patient an immunosuppressive agent, such as a proteasome inhibitor. One such proteasome inhibitor known in the art, for instance as disclosed in U.S. Pat. No. 9,169,492 and U.S. patent application Ser. No. 15/796,137, both of which are incorporated herein by reference, is bortezomib. In another embodiment, an immunosuppressive agent can be an antibody, including polyclonal, monoclonal, scfv or other antibody derived molecule that is capable of suppressing the immune response, for instance, through the elimination or suppression of antibody producing cells. In a further embodiment, the immunosuppressive element can be a short hairpin RNA (shRNA). In such an embodiment, the coding region of the shRNA is included in the rAAV cassette and is generally located downstream, 3′ of the poly-A tail. The shRNA can be targeted to reduce or eliminate expression of immunostimulatory agents, such as cytokines, growth factors (including transforming growth factors β1 and β2, TNF and others that are publicly known).

In some embodiments, the immune modulator is an immunoglobulin degrading enzyme such as IdeS, IdeZ, IdeS/Z, Endo S, or, their functional variant. Non-limiting examples of references of such immunoglobulin degrading enzymes and their uses as described in U.S. Pat. Nos. 7,666,582, 8,133,483, US 20180037962, US 20180023070, US 20170209550, U.S. Pat. No. 8,889,128, WO2010/057626, U.S. Pat. Nos. 9,707,279, 8,323,908, US 20190345533, US 20190262434, and WO2020/016318, each of which are incorporated in their entirety by reference.

In some embodiments, the immune modulator or immunosuppressive agent is a proteasome inhibitor. In certain aspects, the proteasome inhibitor is Bortezomib. In some aspects of the embodiment, the immune modulator comprises bortezomib and anti CD20 antibody, Rituximab. In other aspects of the embodiment, the immune modulator comprises bortezomib, Rituximab, methotrexate, and intravenous gamma globulin. Non-limiting examples of such references, disclosing proteasome inhibitors and their combination with Rituximab, methotrexate and intravenous gamma globulin, as described in U.S. Pat. Nos. 10,028,993, 9,592,247, and 8,809,282, each of which are incorporated in their entirety by reference. One such proteasome inhibitor known in the art, for instance as disclosed in U.S. Pat. No. 9,169,492 and U.S. patent application Ser. No. 15/796,137, both of which are incorporated herein by reference, is bortezomib.

In another embodiment, an immunosuppressive agent can be an antibody, including polyclonal, monoclonal, scfv or other antibody derived molecule that is capable of suppressing the immune response, for instance, through the elimination or suppression of antibody producing cells. In a further embodiment, the immunosuppressive element can be a short hairpin RNA (shRNA). In such an embodiment, the coding region of the shRNA is included in the rAAV cassette and is generally located downstream, 3′ of the poly-A tail. The shRNA can be targeted to reduce or eliminate expression of immunostimulatory agents, such as cytokines, growth factors (including transforming growth factors β1 and β2, TNF and others that are publicly known).

In alternative embodiments, the immune modulator is an inhibitor of the NF-kB pathway. In certain aspects of the embodiment, the immune modulator is Rapamycin, or a functional variant. Non-limiting examples of references disclosing rapamycin and its use described in U.S. Pat. No. 10,071,114, US 20160067228, US 20160074531, US 20160074532, US 20190076458, U.S. Pat. No. 10,046,064, are incorporated in their entirety. In other aspects of the embodiment, the immune modulator is synthetic nanocarriers comprising an immunosuppressant. Non limiting examples of references of immunosuppresants, immunosuppressants coupled to synthetic nanocarriers, synthetic nanocarriers comprising rapamycin, and/or, toloregenic synthetic nanocarriers, their doses, administration and use as described in US20150320728, US 20180193482, US 20190142974, US 20150328333, US20160243253, U.S. Pat. No. 10,039,822, US 20190076522, US 20160022650, U.S. Pat. Nos. 10,441,651, 10,420,835, US 20150320870, US 2014035636, U.S. Pat. Nos. 10,434,088, 10,335,395, US 20200069659, U.S. Pat. No. 10,357,483, US 20140335186, U.S. Pat. Nos. 10,668,053, 10,357,482, US 20160128986, US 20160128987, US 20200038462, US 20200038463, each of which are incorporated in their entirety by reference.

In some embodiments, the immune modulator is synthetic nanocarriers comprising rapamycin (ImmTOR™ nanoparticles) (Kishimoto, et al., 2016, Nat Nanotechnol, 11(10): 890-899; Maldonado, et al., 2015, PNAS, 112(2): E156-165), as disclosed in US20200038463, U.S. Pat. No. 9,006,254 each of which is incorporated herein in its entirety. In some embodiments, the immune modulator is an engineered cell, e.g., an immune cell that has been modified using SQZ technology as disclosed in WO2017192786, which is incorporated herein in its entirety by reference.

In some embodiments, the immune modulator is selected from the group consisting of poly-ICLC, 1018 ISS, aluminum salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, JuvImmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PEPTEL, vector system, PLGA microparticles, resiquimod, SRL172, Virosomes and other Virus-like particles, YF-17D, VEGF trap, R848, beta-glucan, Pam3Cys, and Aquila's QS21 stimulon. In another further embodiment, the immunomodulator or adjuvant is poly-ICLC.

In some embodiments, the immune modulator is a small molecule that inhibit the innate immune response in cells, such as chloroquine (a TLR signaling inhibitor) and 2-aminopurine (a PKR inhibitor), can also be administered in combination with the composition comprising at least one rAAV as disclosed herein. Some non-limiting examples of commercially available TLR-signaling inhibitors include BX795, chloroquine, CLI-095, OxPAPC, polymyxin B, and rapamycin (all available for purchase from INVIVOGEN™). In addition, inhibitors of pattern recognition receptors (PRR) (which are involved in innate immunity signaling) such as 2-aminopurine, BX795, chloroquine, and H-89, can also be used in the compositions and methods comprising at least one rAAV vector as disclosed herein for in vivo protein expression as disclosed herein.

In some embodiments, a rAAV vector can also encode a negative regulators of innate immunity such as NLRX1. Accordingly, in some embodiments, a rAAV vector can also optionally encode one or more, or any combination of NLRX1, NS1, NS3/4A, or A46R. Additionally, in some embodiments, a composition comprising at least one rAAV vector as disclosed herein can also comprise a synthetic, modified-RNA encoding inhibitors of the innate immune system to avoid the innate immune response generated by the tissue or the subject.

In some embodiments, an immune modulator for use in the administration methods as disclosed herein is an immunosuppressive agent. As used herein, the term “immunosuppressive drug or agent” is intended to include pharmaceutical agents which inhibit or interfere with normal immune function. Examples of immunosuppressive agents suitable with the methods disclosed herein include agents that inhibit T-cell/B-cell costimulation pathways, such as agents that interfere with the coupling of T-cells and B-cells via the CTLA4 and B7 pathways, as disclosed in U.S. Patent Pub. No 2002/0182211. In one embodiment, an immunosuppressive agent is cyclosporine A. Other examples include myophenylate mofetil, rapamicin, and anti-thymocyte globulin. In one embodiment, the immunosuppressive drug is administered in a composition comprising at least one rAAV vector as disclosed herein, or can be administered in a separate composition but simultaneously with, or before or after administration of a composition comprising at least one rAAV vector according to the methods of administration as disclosed herein. An immunosuppressive drug is administered in a formulation which is compatible with the route of administration and is administered to a subject at a dosage sufficient to achieve the desired therapeutic effect. In some embodiments, the immunosuppressive drug is administered transiently for a sufficient time to induce tolerance to the rAAV vector as disclosed herein.

Various methods are known to result in the immunosuppression of an immune response of a patient being administered rAAV. Methods known in the art include administering to the patient an immunosuppressive agent, such as a proteasome inhibitor. One such proteasome inhibitor known in the art, for instance as disclosed in U.S. Pat. No. 9,169,492 and U.S. patent application Ser. No. 15/796,137, both of which are incorporated herein by reference, is bortezomib. In some embodiments, an immunosuppressive agent can be an antibody, including polyclonal, monoclonal, scfv or other antibody derived molecule that is capable of suppressing the immune response, for instance, through the elimination or suppression of antibody producing cells. In a further embodiment, the immunosuppressive element can be a short hairpin RNA (shRNA). In such an embodiment, the coding region of the shRNA is included in the rAAV cassette and is generally located downstream, 3′ of the poly-A tail. The shRNA can be targeted to reduce or eliminate expression of immunostimulatory agents, such as cytokines, growth factors (including transforming growth factors β1 and β2, TNF and others that are publicly known).

The use of such immune modulating agents facilitates the ability to for one to use multiple dosing (e.g., multiple administration) over numerous months and/or years. This permits for using multiple agents as discussed below, e.g., a rAAV vector encoding multiple genes, or multiple administrations to the subject.

Manufacturing of the rAAV of the Invention:

In some aspects of the invention, the recombinant AAV comprising a nucleic acid encoding FVIII is produced by the triple transfection method that uses close ended linear duplexed DNA molecules that lack bacterial backbone sequences, for example, as described in International Patent Application No. PCT/US2021/013689, published as WO/2021/146591, which is incorporated herein by reference in its entirety. In some embodiments, the rAAV of the invention is manufactured where one or more, or all of nucleic acids, e.g., AAV rep-cap, Adenovirus helper, and transgene, used as starting material are plasmid. In some embodiments, the rAAV of the invention is manufactured where one or more, or all of nucleic acids, e.g., AAV rep-cap, Adenovirus helper, and transgene, used as starting materials are close ended linear duplexed DNA. One example of close ended linear duplexed DNA is dumbbell shaped DNA. Another example of close ended linear duplexed DNA is doggy bone DNA. Non-limiting examples of methods describing cell free in vitro synthesis of dumbbell-shaped DNA and doggy bone DNA are described in U.S. Pat. No. 6,451,563; Efficient production of superior dumbbell-shaped DNA minimal vectors for small hairpin RNA expression-Nucleic Acids Res. 2015 Oct. 15; 43(18): e120; High-Purity Preparation of a Large DNA Dumbbell-Antisense & nucleic acid drug development 11:149-153 (2001); U.S. Pat. Nos. 9,109,250; 9,499,847; 10,501,782; and WO 2018033730 A1; all of which are herein incorporated by reference in their entireties. The DNA from cell free in vitro synthesis is devoid of any prokaryotic DNA modifications (e.g., is substantially free of bacterial DNA).

In some aspects of the invention, the recombinant AAV comprising a nucleic acid encoding FVIII is produced by the method as described in PCT/US2022/013279, published as WO/2022/159679, which is incorporated herein by reference in its entirety.

### Pharmaceutical Compositions

The rAAV vectors containing a codon-optimized nucleic acid encoding a human FVIII polypeptide as disclosed herein, for use in the methods of administration as disclosed herein can be formulated in a pharmaceutical composition with a pharmaceutically acceptable excipient, i.e., one or more pharmaceutically acceptable carrier substances and/or additives, e.g., buffers, carriers, excipients, stabilizers, etc. The pharmaceutical composition may be provided in the form of a kit. Pharmaceutical compositions comprising the rAAV vectors as disclosed herein for use in the methods of administration as disclosed herein and uses thereof are known in the art.

Accordingly, a further aspect of the invention provides a pharmaceutical composition comprising a rAAV vector containing a codon-optimized nucleic acid encoding a human FVIII polypeptide as disclosed herein, for use in the methods of administration as disclosed herein. Relative amounts of the active ingredient (e.g., a rAAV vectors as disclosed herein), a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1 percent and 99 percent (w/w) of the active ingredient. By way of example, the composition may comprise between 0.1 percent and 100 percent, e.g., between 0.5 and 50 percent, between 1-30 percent, between 5-80 percent, at least 80 percent (w/w) active ingredient.

The pharmaceutical compositions can be formulated using one or more excipients or diluents to (1) increase stability; (2) increase cell transfection or transduction; (3) permit the sustained or delayed release of the payload; (4) alter the biodistribution (e.g., target the viral particle to specific tissues or cell types); (5) increase the translation of encoded protein; (6) alter the release profile of encoded protein and/or (7) allow for regulatable expression of the payload of the invention. In some embodiments, a pharmaceutically acceptable excipient may be at least 95 percent, at least 96 percent, at least 97 percent, at least 98 percent, at least 99 percent, or 100 percent pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia. Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21 st Edition, A. R. Gennaro, Lippincott, Williams and Wilkins, Baltimore, MD, 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

### Compositions/Formulations

The rAAV vectors containing a codon-optimized nucleic acid encoding a human FVIII polypeptide as disclosed herein, can be formulated in a composition. For example, the rAAV vectors as disclosed herein can be formulated in a pharmaceutical composition with a pharmaceutically acceptable excipient, i.e., one or more pharmaceutically acceptable carrier substances and/or additives, e.g., buffers, carriers, excipients, stabilisers, etc. The composition, e.g., the pharmaceutical composition may be provided in the form of a kit. It is noted the terms “composition” and “formulation” are used interchangeably here.

Accordingly, in one aspect, provided herein is a composition comprising the recombinant AAV vector particles described herein. Generally, the composition comprises the recombinant AAV vector particles described herein at a concentration from about 1e⁹ vg/ml to about 1e¹⁵ vg/ml. In some embodiments, the composition comprises the recombinant AAV vector particles described herein at a concentration from about 1e¹⁰ vg/ml to about 1e¹⁴ vg/ml. In some embodiments, the composition comprises the recombinant AAV vector particles described herein at a concentration from about 1e¹² vg/ml to about 1e¹⁴ vg/ml. In some embodiments, the composition comprises the recombinant AAV vector particles described herein at a concentration from about 1e¹² vg/ml to about 1e¹⁵ vg/ml. For example, the composition comprises the recombinant AAV vector particles described herein at a concentration from about 3e¹² vg/ml to about 3e¹³ vg/ml, from about 2.5e¹² vg/ml to about 1e¹⁴ vg/ml, from about 3e¹³ vg/ml to about 1e¹⁴ vg/ml, or from 1e¹³ vg/ml to about 1e¹⁴ vg/ml.

In some embodiments, the composition comprises the recombinant AAV vector particles described herein at a concentration of about 1e¹² vg/ml, or about 1.5e¹² vg/ml, or about 2e¹² vg/ml, or about 2.5e¹² vg/ml, or about 3e¹² vg/ml, or about 3.5e¹² vg/ml, or about 4e¹² vg/ml, or about 4.5e¹² vg/ml, or about 5e¹² vg/ml, or about 5.5e¹² vg/ml, or about 6e¹² vg/ml, or about 6.5e¹² vg/ml, or about 7e¹² vg/ml, or about 7.5e¹² vg/ml, or about 8e¹² vg/ml, or about 8.5e¹² vg/ml, or about 9e¹² vg/ml, or about 9.5e¹³ vg/ml, or about 1e¹³ vg/ml, or about 1.5e¹³ vg/ml, or about 2e¹³ vg/ml, or about 2.5e¹³ vg/ml, or about 3e¹³ vg/ml, or about 3.5e¹³ vg/ml, or about 4e¹³ vg/ml, or about 4.5e¹³ vg/ml, or about 5e¹³ vg/ml, or about 5.5e¹³ vg/ml, or about 6e¹³ vg/ml, or about 6.5e¹³ vg/ml, or about 7e¹³ vg/ml, or about 7.5e¹³ vg/ml, or about 8e¹³ vg/ml, or about 8.5e¹³ vg/ml, or about 9e¹³ vg/ml, or about 9.5e¹³ vg/ml, or about 1e¹⁴ vg/ml.

The pharmaceutical composition comprises the population of purified recombinant adeno-associated virus (rAAV) described herein. The pharmaceutical composition comprising the rAAV, comprises a buffer of pH about 6.5 to about 8.0. In some embodiments, the pH is about 6.5 to about 7.5. For example, the pH is from about 6.5, about 6.6, about 6.7, about 6.8, about 6.9, about 7.0, about 7.1, about 7.2, about 7.3, about 7.4 or about 7.5. In some preferred embodiments, the pH is less than about 7.5. For example, the pH is less than about 7.4, less than about 7.3, less than about 7.2, less than about 7.1, less than about 7.0, less than about 6.9, less than about 6.8, less than about 6.7, or less than about 6.6. In some embodiments, the pharmaceutical composition comprises one or, more excipients, comprising one or, more multivalent ions and/or, salts thereof. In some embodiments, the multivalent ions can be selected or, optionally selected from the group consisting of citrate, sulfate, magnesium and phosphate. In some embodiments, the pharmaceutical composition comprises one or, more excipients, comprising one or, more ions selected or, optionally selected from the group consisting of, sodium, potassium, chroride, ammonium, carbonate, nitrate, chlorate, chlorite, and calcium. In some embodiments, the pharmaceutical composition comprising the rAAV, further comprises a non-ionic surfactant. In some embodiments, the non-ionic surfactant is selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof. In some embodiments, non-ionic surfactant is selected from the group consisting of TWEEN 60 nonionic detergent, PPG-PEG-PPG Pluronic 10R5, Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P338 IGEPAL CO-720, IGEPAL CO-630, IGEPAL CA-720, Brij S20, BrijS10, Brij 010, Brij C10, BRIJ 020, ECOSURF EH-9, ECOSURF EH-14, TERGITOL 15-S-7, PF-68, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof. In some embodiments, the pharmaceutical composition further comprises polyol, or, sugar, or similar. See, e.g., International Patent No. WO2022/159679, which is incorporated herein by reference in its entirety.

In some embodiments, the composition comprises a buffer. It is noted that any physiological buffer can be used. Non-limiting examples of buffers include, but are not limited to, PBS, Tris.HCl, phosphate, citric acid, histidine, tromethamine, succinic acid, malic acid, α-ketoglutaric acid, carbonate (bicarbonate-carbonic acid buffer), and protein buffers. In some embodiments, the buffer is PBS. In some embodiments, the buffer comprises Tris. In some embodiments, buffer is Tris.HCl. In some embodiments, the buffer is histidine buffer.

Generally, the buffer has a salt concentration of from about 50 mM to about 750 mM. For example, the buffer has a salt concentration from about 75 mM to about 700 mM, from about 100 mM to about 650 mM, from about 120 mM to about 600 mM, or from about 140 mM to about 550 mM. In some embodiments, the buffer has a salt concentration from about 150 mM to about 400 mM. In some embodiments, the buffer has a salt concentration of about 150 mM, about 175 mM, about 200 mM, about 225 mM, about 250 mM, about 275 mM, about 300 mM, about 325 mM, about 350 mM, about 375 mM, about 400 mM, about 425 mM, about 450 mM, or about 475 mM. In some preferred embodiments, the buffer has a salt concentration of about 150 mM, about 200 mM or about 365 mM.

In some embodiments, the ionic strength of the composition is at least about 100 mM. For example, the ionic strength of the composition is from about 125 mM to about 750 mM, or from about 150 mM to about 500 mM, or from about 175 mM to about 700 mM, from about 200 mM to about 600 mM, or from about 225 mM to about 550 mM, or from about 250 mM to about 500 mM, or from about 275 mM to about 450 mM, or from about 300 mM to about 400 mM. In some embodiments, the ionic strength of the composition is at least about 125 mM, at least about 150 mM, at least about 175 mM, at least about 200 mM, at least about 225 mM, at least about 250 mM, at least about 275 mM, at least about 300 mM, at least about 325 mM, at least about 350 mM, at least about 375 mM, at least about 400 mM, at least about 425 mM, at least about 450 mM, at least about 475 mM or at least about 500 mM. In some embodiments, the ionic strength of the composition is less than 100 mM, for example about 95 mM, about 90 mM, about 85 mM, about 80 mM, about 75 mM, about 70 mM, about 65 mM, about 60 mM, about 55 mM, about 50 mM, or even less.

The osmolarity of the composition is maintained at near isotonic levels. For example, the osmolarity of the composition can be from about 100 mOsm to about 600 mOsm, such as from about 125 mOsm to about 500 mOsm, or, from about 130 mOsm to about 350 mOsm, or, from about 140 mOsm to about 400 mOsm, or, from about 140 mOsm to about 350 mOsm, or from about 200 mOsm to about 400 mOsm, or from about 500 mOsm to about 600 mOsm, or from about 200 mOsm to about 600 mOsm, or from about 300 mOsm to about 600 mOsm, or from about 200 mOsm to about 500 mOsm, or from about 300 mOsm to about 400 mOsm, or from about 150 mOsm to about 350 mOsm, or from about 175 mOsm to about 300 mOsm, or from about 300 mOsm to about 375 mOsm, or from about 200 mOsm to about 350 mOsm, or from about 225 mOsm to about 325 mOs, or from about 525 mOsm to about 590 mOsm. In some embodiments, the composition comprises an isotonic solution.

Generally, the composition has a pH of about 6.5 to about 8.0. For example, the composition has a pH of about 6.5 to about 7.5. In some embodiments, the composition has a pH of from about 7 to about 8. For example, the composition has a pH of from about 7.3 to about 7.9. In some other non-limiting example, the composition has a pH of from about 7.4 to about 7.8 or from about 7.4 to about 7.7. In some embodiments, the composition has a pH of from about 7.3 to about 7.6, e.g., from about 7.3 to about 7.55. In some preferred embodiments, the composition has a pH less than about 7.5. For example, the composition has a pH about 7.4 or lower, about 7.3 or lower, about 7.2 or lower, about 7.1 or lower, about 7.0 or lower, about 6.9 or lower, about 6.8 or lower, about 6.7 or lower, about 6.6 or lower, or about 6.5 or lower.

Generally, the composition has a pH of about 6.5 to about 8.0. For example, the composition has a pH of about 6.5 to about 7.5. In some embodiments, the composition has a pH of from about 7 to about 8. For example, the composition has a pH of from about 7.3 to about 7.9. In some other non-limiting example, the composition has a pH of from about 7.4 to about 7.8 or from about 7.4 to about 7.7. In some embodiments, the composition has a pH of from about 7.3 to about 7.6, e.g., from about 7.3 to about 7.55. In some preferred embodiments, the composition has a pH less than about 7.5. For example, the composition has a pH about 7.4 or lower, about 7.3 or lower, about 7.2 or lower, about 7.1 or lower, about 7.0 or lower, about 6.9 or lower, about 6.8 or lower, about 6.7 or lower, about 6.6 or lower, or about 6.5 or lower.

The composition can comprise one or more ions and/or salts thereof. Exemplary ions include, but are not limited to sodium, potassium, chloride, magnesium ammonium, carbonate, nitrate, chlorate, chlorite, and calcium. The ions can be provided as a salt, such as a halide (F, Cl, Br, I) salt of sodium, potassium, magnesium, and/or calcium, non-limiting examples of which include NaCl, KCl, MgCl₂, CaCl₂, and combinations thereof. Additional exemplary salts that can be used include, but are not limited to, carboxylic acid salts, such as acetates, propionates, pyrrol idonecarboxylates (or pidolates) or sorbates; poly hydroxylated carboxylic acid salts, such as gluconates, heptagluconates, ketogluconates, lactate gluconates, ascorbates or pantothenates; mono- or polycarboxyl hydroxy acid salts, such as citrates or lactates; amino acid salts, such as aspartates or glutamates; and fulvate salts. The salts are individually included at a concentration of from about 500 μM to about 500 mM.

In some embodiments, the composition comprises one or more multivalent ions and/or salts thereof. Exemplary multivalent ions include, but are not limited to, calcium, citrate, sulfate, magnesium, and phosphate. Multivalent ions and/or salts thereof can be individually included in the composition at a concentration of from about 500 μM to about 500 mM, for example, at a concentration of about 500 μM, about 750 μM, about 1 mM, about 1.3 mM, about 1.5 mM, about 1.7 mM, about 2.3 mM, about 2.5 mM, about 2.7 mM, about 3.3 mM, about 3.5 mM, about 3.7 mM, about 4.3 mM, about 4.5 mM, about 4.7 mM, about 5 mM, about 10 mM, about 25 mM, about 50 mM, about 75 mM, about 80 mM, about 85 mM, about 90 mM, about 95 mM, about 100 mM, about 125 mM, about 150 mM, about 175 mM, about 200 mM, about 225 mM, about 250 mM, about 275 mM, about 300 mM, about 325 mM, about 350 mM, about 375 mM, about 400 mM, about 425 mM, about 450 mM, about 475 mM, or about 500 mM. Non limiting examples of salts are NaCl, KCl, CaCl₂, CaSO₄, MgSO₄, Na₃PO₄, CaCO₃, NaNO₃, Al₂(SO₄)₃.

In some embodiments, the composition comprises NaCl. When present, NaCl can be at a concentration from about 100 mM to about 500 mM, or from about 125 mM to about 450 mM, or from about 100 mM to about 200 mM, or from about 150 mM to about 200 mM. For example, the composition can comprise NaCl at a concentration from about 150 mM to about 425 mM, from about 175 mM to about 400 mM, or from about 175 mM to about 375 mM, or from about 200 mM to about 375 mM.

In some embodiments, the composition comprises KCl. When present, KCl can be at a concentration from about 1 mM to about 10 mM. For example, the composition can comprise KCl at a concentration from about 1.5 mM to about 7.5 mM.

In some embodiments, the composition comprises CaCl₂. When present, CaCl₂ can be at a concentration from about 0.1 mM to about 2 mM. For example, the composition can comprise CaCl₂ at a concentration from about 0.5 mM to about 1.5 mM. In some embodiments, the composition comprises CaCl₂ at a concentration from about 0.75 mM to about 1.25 mM.

In some embodiments, the composition comprises MgCl₂. When present, MgCl₂ can be at a concentration from about 0.1 mM to about 1.5 mM. For example, the composition can comprise MgCl₂ at a concentration from about 0.25 mM to about 1 mM or from about 0.25 mM to about 0.75 mM.

In some embodiments, the composition comprises MgSO₄. When present, MgSO₄ can be at a concentration from about 5 mM to about 150 mM. For example, the composition can comprise MgSO₄ at a concentration from about 10 mM to about 120 mM, or from about 10 mM to about 50 mM, or from about 15 mM to about 45 mM, or about 75 mM to about 125 mM, or from about 80 mM to about 100 mM, or from about 85 mM to about 95 mM, or from about 15 mM to about 100 mM.

In some embodiments, the composition comprises phosphate, e.g., mono basic or dibasic phosphate or a salt thereof. When present, the phosphate, e.g., mono basic or dibasic phosphate or a salt thereof can be at a concentration from about 5 mM to about 30 mM. For example, the composition can comprise phosphate, e.g., mono basic or dibasic phosphate or a salt thereof at a concentration from about 7.5 mM to about 25 mM. In some embodiments, the composition comprises phosphate, e.g., mono basic or dibasic phosphate or a salt thereof at a concentration from about 10 mM to about 20 mM.

In some embodiments, the composition comprises a mono basic phosphate or a salt thereof at a concentration from about 0.25 mM to about 3 mM. For example, the composition comprises a mono basic phosphate or a salt thereof at a concentration from about 0.5 mM to about 2.75 mM, or from about 0.75 mM to about 2.5 mM or from about 1 mM to about 2.25 mM. In some embodiments, the mono basic phosphate or salt thereof is potassium phosphate monobasic.

In some embodiments, the composition comprises a dibasic phosphate or a salt thereof at a concentration from about 5 mM to about 15 mM. For example, the composition comprises a dibasic phosphate or a salt thereof at a concentration from about 7.5 mM to about 12.5 mM or from about 8 mM to about 10 mM. In some embodiments, the dibasic phosphate or a salt thereof is sodium phosphate dibasic. In some embodiments, the composition is substantially free of dibasic phosphate, e.g., sodium phosphate dibasic.

In some embodiments, the composition comprises Tris (e.g., Tris.HCl) or a salt thereof at a concentration from about 1 mM to about 50 mM. For example, the composition comprises Tris (e.g., Tris.HCl) or a salt thereof at a concentration of from about 5 mM to about 40 mM, or from about 7.5 mM to about 35 mM, or from about 10 mM to about 30 mM or from about 15 mM to about 25 mM.

In some embodiments, the composition comprises histidine or a salt thereof at a concentration from about 1 mM to about 50 mM. For example, the composition comprises histidine or a salt thereof at a concentration of from about 5 mM to about 40 mM, or from about 7.5 mM to about 35 mM, or from about 10 mM to about 30 mM or from about 15 mM to about 25 mM.

The composition can also comprise a bulking agent. Exemplary bulking agents include, but are not limited to sugars, polyols and (PVP K24). Exemplary polyols include, but are not limited to, polyhydroxy hydrocarbons, monosaccharides, disaccharides, and trisaccharides. Some exemplary polyols include but are not limited to, sorbitol, mannitol, glycerol, propylene glycol, polyethylene glycol, dulcitol, sucrose, lactose, maltose, trehalose and dextran. In some embodiments, polyol is sorbitol, sucrose or mannitol. In some embodiments, the bulking agent is sorbitol. In some embodiments, the bulking agent is sucrose. In some embodiments, the bulking agent is mannitol. In some embodiments, the bulking agent is trehalose, e.g., trehalose dehydrate. In some embodiments, the bulking agent is a dextran, e.g., Dextran T40 and/or Dextran T10.

When present, the bulking agent can be present at a concentration of from about 0.5% (w/v) to about 10% (w/v). For example, the composition can comprise a bulking agent, e.g., a polyol or providone (PVP K24) at a concentration from about from about 1% (w/v) to about 7.5% (w/v), e.g., from about 1% (w/v) to about 4% (w/v) or from about 4% (w/v) to about 6% (w/v).

In some embodiments, the composition comprises glycerol, sorbitol, sucrose, or mannitol at a concentration from about 1% (w/v) to about 10% (w/v). In some embodiments, the composition comprises glycerol, sorbitol, sucrose, or mannitol at a concentration from about 1% (w/v) to about 10% (w/v). In some embodiments, the composition comprises sorbitol at concentration from about 3% (w/v) to about 6% (w/v). In some embodiments, the composition comprises sorbitol at concentration of about 1% (w/v), about 2% (w/v), about 3% (w/v), about 4% (w/v), about 5% (w/v), about 6% (w/v), about 7% (w/v), about 8% (w/v), about 9% (w/v), or about 10% (w/v). In some embodiments, the composition comprises sucrose at concentration from about 3% (w/v) to about 6% (w/v). In some embodiments, the composition comprises sucrose at concentration of about 1% (w/v), about 2% (w/v), about 3% (w/v), about 4% (w/v), about 5% (w/v), about 6% (w/v), about 7% (w/v), about 8% (w/v), about 9% (w/v), or about 10% (w/v). In some embodiments, the composition comprises mannitol at concentration from about 3% (w/v) to about 6% (w/v). In some embodiments, the composition comprises mannitol at concentration of about 1% (w/v), about 2% (w/v), about 3% (w/v), about 4% (w/v), about 5% (w/v), about 6% (w/v), about 7% (w/v), about 8% (w/v), about 9% (w/v), or about 10% (w/v).

The composition can also comprise a non-ionic surfactant. The non-ionic surfactant can be selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof. Non-limiting examples of suitable non-ionic surfactants include polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and combinaitons thereof. In some embodiments, the non-ionic surfactant is selected from the group consisting of TWEEN 60 nonionic detergent, PPG-PEG-PPG Pluronic 10R5, Pluronic F-68 (PF 68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, BrijS10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof. In some embodiments, the non-ionic surfactant is Poloxamer P 188, Poloxamer P407, Pluronic F-68, Ecosurf SA-15, Brij S20, Tergitol NP-10, IGEPAL CA 720 or Tween 80. In some embodiments, the composition is substantially free of a non-ionic surfactant. In some embodiments, the non-ionic surfactant is not a polysorbate, e.g., Tween 80 (also referred to as polysorbate 80 or PS80).

When present, the non-ionic surfactant can be present at a concentration from about 0.0001% (w/v) to about 0.01% (w/v). For example, the composition can comprise a non-ionic surfactant at a concentration from about 0.0005% (w/v) to about 0.0015% (w/v). In some embodiments, the composition can comprise a non-ionic surfactant at a concentration of about 0.0001% (w/v), about 0.0002% (w/v), about 0.0003% (w/v), about 0.0004% (w/v), about 0.0005% (w/v), about 0.0006% (w/v), about 0.0007% (w/v), about 0.0008% (w/v), about 0.0009% (w/v), about 0.001% (w/v), about 0.002% (w/v), about 0.003% (w/v), about 0.004% (w/v), about 0.005% (w/v), about 0.006% (w/v), about 0.007% (w/v), about 0.008% (w/v), about 0.009% (w/v), or about 0.01%. (w/v). In some preferred embodiments, the composition comprises a non-ionic surfactant at a concentration of about 0.0005% (w/v) or about 0.001% (w/v).

In some embodiments, the composition comprises, in addition to the rAAV, a buffer (e.g., PBS, Tris.HCl, phosphate, citric acid, histidine, tromethamine, succinic acid, malic acid, α-ketoglutaric acid, carbonate buffer), a bulking agent (e.g., a polyol such as sorbitol, mannitol, glycerol, propylene glycol, polyethylene glycol, dulcitol, sucrose, lactose, maltose, trehalose and dextran) and a non-ionic surfactant (e.g., Poloxamer P 188, Poloxamer P407, Pluronic F-68, Ecosurf SA-15, Brij S20, Tergitol NP-10, IGEPAL CA 720 or Tween 80).

In some embodiments, the composition comprises, in addition to the rAAV, a buffer (e.g., PBS, Tris.HCl, phosphate, citric acid, histidine, tromethamine, succinic acid, malic acid, α-ketoglutaric acid, carbonate buffer), a bulking agent (e.g., a polyol such as sorbitol, mannitol, glycerol, propylene glycol, polyethylene glycol, dulcitol, sucrose, lactose, maltose, trehalose and dextran), a non-ionic surfactant (e.g., Poloxamer P 188, Poloxamer P407, Pluronic F-68, Ecosurf SA-15, Brij S20, Tergitol NP-10, IGEPAL CA 720 or Tween 80), and a multivalent ion (e.g., a multivalent ion selected from the group consisting of calcium, citrate, sulfate, and magnesium).

In some embodiments, the composition comprises, in addition to the rAAV, a buffer (e.g., PBS, Tris.HCl, phosphate, citric acid, histidine, tromethamine, succinic acid, malic acid, α-ketoglutaric acid, carbonate buffer), a bulking agent (e.g., a polyol such as sorbitol, mannitol, glycerol, propylene glycol, polyethylene glycol, dulcitol, sucrose, lactose, maltose, trehalose and dextran), and a multivalent ion (e.g., a multivalent ion selected from the group consisting of calcium, citrate, sulfate, and magnesium).

It is noted that any one of the specific buffers or group of buffers listed in the description of the compositions can be used with any one of the specific bulking agents or group of bulking agents listed in the description of the compositions and with any of the specific non-ionic surfactants or group of surfactants listed in the description of the compositions and with any of the specific multivalent ions and multivalent ion group listed in the description of the compositions. Similarly, any one of the specific bulking agents or group of bulking agents listed in the description of the compositions can be used with any one of the specific buffers or group of buffers listed in the description of the compositions and with any of the specific non-ionic surfactants or group of surfactants listed in the description of the compositions and with any of the specific multivalent ions and multivalent ion group listed in the description of the compositions. Likewise, any of the specific non-ionic surfactants or group of surfactants listed in the description of the compositions can be used with any one of the specific buffers or group of buffers listed in the description of the compositions and with any one of the specific bulking agents or group of bulking agents listed in the description of the compositions and with any of the specific multivalent ions and multivalent ion group listed in the description of the compositions. As well, any of the specific multivalent ions and multivalent ion group listed in the description of the compositions can be used with any one of the specific buffers or group of buffers listed in the description of the compositions and with any one of the specific bulking agents or group of bulking agents listed in the description of the compositions and with any of the specific non-ionic surfactants or group of surfactants listed in the description of the compositions. In other words, all individual specific combinations of buffers, buffer group, bulking agents, bulking agent groups, non-ionic surfactants, non-ionic surfactant groups, multivalent ions and multivalent ion groups listed in the description of the compositions are specifically contemplated and claimed.

In yet other embodiments of the present invention, the formulation comprises sodium phosphate, dibasic at a concentration of from about 0.1 mg/ml to about 3 mg/ml, sodium phosphate monobasic monohydrate at a concentration of from about 0.1 mg/ml to about 3 mg/ml, sodium chloride at a concentration of from about 1 mg/ml to about 20 mg/ml, mannitol at a concentration of from about 5 mg/ml to about 40 mg/ml, and poloxamer 188 at a concentration of from about 0.1 mg/ml to about 4 mg/ml. In another embodiment, the formulation of the present invention comprises sodium phosphate, dibasic at a concentration of about 1.42 mg/mL, sodium phosphate monobasic monohydrate at a concentration of about 1.38 mg/ml, sodium chloride at a concentration of about 8.18 mg/ml, mannitol at a concentration of about 20 mg/ml, and poloxamer 188 at a concentration of about 2 mg/ml. The formulations of the present invention may be in liquid form and may comprise the AAV FVIII virus particle at a concentration of from about 1E12 vg/ml to about 2E14 vg/mL, or at a concentration of about 2E13 vg/ml.

In other aspects, the AAV FVIII formulation of the invention comprises one or more pharmaceutically acceptable excipients to provide the formulation with advantageous properties for storage and/or administration to subjects for the treatment of hemophilia A. In certain embodiments, the formulations of the present invention are capable of being stored at ≤65° C. for a period of at least 2 weeks, at least 4 weeks, at least 6 weeks and at least about 8 weeks, without detectable change in stability. In this regard, the term “stable” means that the recombinant AAV FVIII virus present in the formulation essentially retains its physical stability, chemical stability and/or biological activity during storage. In certain embodiments of the present invention, the recombinant AAV FVIII virus present in the formulation retains at least about 80% of its biological activity in a human patient during storage for a determined period of time at −65° C., or at least about 85%, 90%, 95%, 98% or 99% of its biological activity in a human patient.

In certain aspects, the formulation comprising recombinant AAV FVIII virions further comprises one or more buffering agents. For example, in various aspects, the formulation of the present invention comprises sodium phosphate dibasic at a concentration of about 0.1 mg/ml to about 3 mg/ml, about 0.5 mg/ml to about 2.5 mg/ml, about 1 mg/ml to about 2 mg/ml, or about 1.4 mg/ml to about 1.6 mg/ml. In another embodiment, the AAV FVIII formulation of the present invention comprises about 1.42 mg/ml of sodium phosphate, dibasic (dried). Another buffering agent that may find use in the recombinant AAV FVIII formulations of the present invention is sodium phosphate, monobasic monohydrate which, in some embodiments, finds use at a concentration of from about 0.1 mg/ml to about 3 mg/m, about 0.5 mg/ml to about 2.5 mg/ml, about 1 mg/ml to about 2 mg/ml, or about 1.3 mg/ml to about 1.5 mg/ml. In one embodiment, the AAV FVIII formulation of the present invention comprises about 1.38 mg/ml of sodium phosphate, monobasic monohydrate. In another embodiment of the present invention, the recombinant AAV FVIII formulation of the present invention comprises about 1.42 mg/ml of sodium phosphate, dibasic and about 1.38 mg/ml of sodium phosphate, monobasic monohydrate.

In another aspect, the recombinant AAV FVIII formulation of the present invention may comprise one or more isotonicity agents, such as sodium chloride, at a concentration of about 1 mg/ml to about 20 mg/ml, for example, about 1 mg/ml to about 10 mg/ml, about 5 mg/ml to about 15 mg/m, or about 8 mg/ml to about 20 mg/ml. In one embodiment, the formulation of the present invention comprises about 8.18 mg/ml sodium chloride. Other buffering agents and isotonicity agents, known in the art are suitable and may be routinely employed for use in the formulations of the present disclosure.

In another aspect, the recombinant AAV FVIII formulations of the present invention may comprise one or more bulking agents. Exemplary bulking agents include without limitation mannitol, sucrose, dextran, lactose, trehalose, and povidone (PVP K24). In some embodiments, the formulations of the present invention comprise mannitol, which may be present in an amount from about 5 mg/ml to about 40 mg/ml, or from about 10 mg/ml to about 30 mg/mil, or from about 15 mg/ml to about 25 mg/ml. In one embodiment, mannitol is present at a concentration of about 20 mg/ml.

In yet another aspect, the recombinant AAV FVIII formulations of the present invention may comprise one or more surfactants, which may be non-ionic surfactants. Exemplary surfactants include ionic surfactants, non-ionic surfactants, and combinations thereof. For example, the surfactant can be, without limitation, TWEEN 80 (also known as polysorbate 80, or its chemical name polyoxyethylene sorbitan monooleate), sodium dodecylsulfate, sodium stearate, ammonium lauryl sulfate, TRITON AG 98 (Rhone-Poulenc), poloxamer 407, poloxamer 188 and the like, and combinations thereof. In one embodiment, the formulation of the present invention comprises poloxamer 188, which may be present at a concentration of from about 0.1 mg/ml to about 4 mg/ml, or from about 0.5 mg/ml to about 3 mg/ml, from about 1 mg/ml to about 3 mg/ml, about 1.5 mg/ml to about 2.5 mg/ml, or from about 1.8 mg/ml to about 2.2 mg/ml. In one embodiment, poloxamer 188 is present at a concentration of about 2.0 mg/ml.

The recombinant AAV FVIII virus-containing formulations of the present disclosure are stable and can be stored for extended periods of time without an unacceptable change in quality, potency, or purity. In one aspect, the formulation is stable at a temperature of about 5° C. (e.g., 2° C. to 8° C.) for at least 1 month, for example, at least 1 month, at least 3 months, at least 6 months, at least 12 months, at least 18 months, at least 24 months, or more. In another aspect, the formulation is stable at a temperature of less than or equal to about −20° C. for at least 6 months, for example, at least 6 months, at least 12 months, at least 18 months, at least 24 months, at least 36 months, or more. In another aspect, the formulation is stable at a temperature of less than or equal to about −40° C. for at least 6 months, for example, at least 6 months, at least 12 months, at least 18 months, at least 24 months, at least 36 months, or more. In another aspect, the formulation is stable at a temperature of less than or equal to about −60° C. for at least 6 months, for example, at least 6 months, at least 12 months, at least 18 months, at least 24 months, at least 36 months, or more.

### Exemplary Compositions

In some embodiments, the composition, e.g., the pharmaceutical composition comprises, in addition to the rAAV, about 10 mM Phosphate pH 7.4, about 200 mM NaCl, about 5 mM KCl, about 1% (w/v) mannitol, and about 0.0005% (w/v) IGEPAL CA 720.

In some embodiments, the composition, e.g., the pharmaceutical composition comprises, in addition to the rAAV, about 20 mM Phosphate pH 7.4, about 300 mM NaCl, about 3 mM KCl, about 3% (w/v) mannitol, and about 0.001% (w/v) Brij S20.

In some embodiments, the composition, e.g., the pharmaceutical composition comprises, in addition to the rAAV, about 20 mM Phosphate pH 7.4, about 300 mM NaCl, about 3 mM KCl, about 3% (w/v) sorbitol, and about 0.001% (w/v) Ecosurf SA-15.

In some embodiments, the composition, e.g., the pharmaceutical composition comprises, in addition to the rAAV, about 10 mM Phosphate pH 7.4, about 350 mM NaCl, about 2.7 mM KCl, about 5% (w/v) sorbitol, and about 0.001% (w/v) poloxamer 188.

In some embodiments, the composition, e.g., the pharmaceutical composition comprises, in addition to the rAAV, about 10 mM Phosphate pH 6.95-7.2, about 137 mM NaCl, about 2.7 mM KCl, about 0.9 mM CaCl₂, about 0.5 mM MgCl₂, and about 0.001% (w/v) Pluronic F-68.

In some embodiments, the composition, e.g., the pharmaceutical composition comprises, in addition to the rAAV, about 10 mM Phosphate pH 7.3, about 180 mM NaCl, about 2.7 mM KCl, about 5% (w/v) sorbitol, and about 0.001% (w/v) Poloxamer 188.

In some embodiments, the composition, e.g., the pharmaceutical composition comprises, in addition to the rAAV, about 15 mM Phosphate pH 7.4, about 375 mM NaCl, about 3.5 mM KCl, about 5% (w/v) sorbitol, and about 0.0005% (w/v) Tergitol NP-10.

In some embodiments, the c composition, e.g., the pharmaceutical composition comprises, in addition to the rAAV, about 15 mM Phosphate pH 7.4, about 375 mM NaCl, about 3.5 mM KCl, about 3% (w/v) glycerol, and about 0.0005% (w/v) Tween 80.

In some embodiments, the composition, e.g., the pharmaceutical composition comprises, in addition to the rAAV, about 9.0 mM Na2HPO4·7H2O, about 1.0 mM KH2PO4 pH 7.4, about 350 mM NaCl, about 2.7 mM KCl, about 5% (w/v) sorbitol, and about 0.001% (w/v) poloxamer 188.

In some embodiments, the composition, e.g., the pharmaceutical composition comprises, in addition to the rAAV, about 10 mM Phosphate pH 7.6, about 137 mM NaCl, about 2.7 mM KCl, about 5% (w/v) sorbitol, and about 0.01% Pluronic F-68.

In some embodiments, the composition, e.g., the pharmaceutical composition comprises, in addition to the rAAV, about 10 mM Phosphate pH 7.4, about 137 mM NaCl, about 2.7 mM KCl, about 5% (w/v) sorbitol, about 0.01% Pluronic F-68, and about 20 mM MgSO₄.

In some embodiments, the composition, e.g., the pharmaceutical composition comprises, in addition to the rAAV, about 10 mM Phosphate pH 7.6, about 137 mM NaCl, about 2.7 mM KCl, about 5% (w/v) mannitol, and about 0.01% Pluronic F-68.

In some embodiments, the composition, e.g., the pharmaceutical composition comprises, in addition to the rAAV, about 10 mM Phosphate pH 7.3, about 137 mM NaCl, about 2.7 mM KCl, about 5% (w/v) mannitol, about 0.01% Pluronic F-68, and about 20 mM MgSO₄.

In some embodiments, the composition, e.g., the pharmaceutical composition comprises, in addition to the rAAV, about 10 mM Phosphate pH 7.4, about 137 mM NaCl, about 2.7 mM KCl, about 5% (w/v) sorbitol, and about 20 mM MgSO₄.

In some embodiments, the composition, e.g., the pharmaceutical composition comprises, in addition to the rAAV, about 10 mM Phosphate pH 7.4, about 137 mM NaCl, about 2.7 mM KCl, about 5% (w/v) mannitol, and about 20 mM MgSO₄.

In some embodiments, the composition, e.g., the pharmaceutical composition comprises recombinant AAV vector (rAAV), in 10 mM Phosphate pH 7.4, 200 mM NaCl, 5 mM KCl, 1% (w/v) mannitol, 0.0005% (w/v) IGEPAL CA 720 to a fill volume of 5 ml. In some embodiments, the fill volume is 1 ml, 2 ml, 3 ml, 4 ml, 5 ml, 6 ml, 7 ml, 8 ml, 9 ml, or 10 ml.

In some embodiments, the composition, e.g., the pharmaceutical composition comprises recombinant AAV vector (rAAV), in 20 mM Phosphate pH 7.4, 300 mM NaCl, 3 mM KCl, 3% (w/v) mannitol, 0.001% (w/v) Brij S20 to a fill volume of 5 ml. In some embodiments, the fill volume is 1 ml, 2 ml, 3 ml, 4 ml, 5 ml, 6 ml, 7 ml, 8 ml, 9 ml, or 10 ml.

In some embodiments, the composition, e.g., the pharmaceutical composition comprises recombinant AAV vector (rAAV), in 20 mM Phosphate pH 7.4, 300 mM NaCl, 3 mM KCl, 3% (w/v) sorbitol, 0.001% (w/v) Ecosurf SA-15 to a fill volume of 5 ml. In some embodiments, the fill volume is 1 ml, 2 ml, 3 ml, 4 ml, 5 ml, 6 ml, 7 ml, 8 ml, 9 ml, or 10 ml.

In some embodiments, the composition, e.g., the pharmaceutical composition comprises recombinant AAV vector (rAAV), in 10 mM Phosphate pH 7.4, 350 mM NaCl, 2.7 mM KCl, 5% (w/v) sorbitol, 0.001% (w/v) poloxamer 188 to a fill volume of 5 ml. In some embodiments, the fill volume is 1 ml, 2 ml, 3 ml, 4 ml, 5 ml, 6 ml, 7 ml, 8 ml, 9 ml, or 10 ml.

In some embodiments, the composition, e.g., the pharmaceutical composition comprises recombinant AAV vector (rAAV), in 15 mM Phosphate pH 7.4, 375 mM NaCl, 3.5 mM KCl, 5% (w/v) sorbitol, 0.0005% (w/v) Tergitol NP-10 to a fill volume of 5 ml. In some embodiments, the fill volume is 1 ml, 2 ml, 3 ml, 4 ml, 5 ml, 6 ml, 7 ml, 8 ml, 9 ml, or 10 ml.

In some embodiments, the composition, e.g., the pharmaceutical composition comprises recombinant AAV vector (rAAV), in 15 mM Phosphate pH 7.4, 375 mM NaCl, 3.5 mM KCl, 3% (w/v) glycerol, 0.0005% (w/v) Tween 80 to a fill volume of 5 ml. In some embodiments, the fill volume is 1 ml, 2 ml, 3 ml, 4 ml, 5 ml, 6 ml, 7 ml, 8 ml, 9 ml, or 10 ml.

In one embodiment, the AAV vector described herein is formulated to a concentration of ≥5.0×10¹² genome copies (CC) AAV/mL in a solution of 20 mM Tris, 1 mM magnesium chloride (MgCl₂)·6H₂O, 200 mM sodium chloride (NaCl), containing 0.01% (Weight to volume) Pluronic® F-68 poloxamer, pH 8.0+/−02, In one embodiment, the formulation is stored as a frozen liquid in a 2 mL 13 mm Type I clear glass vial at ≤−60° C. In one embodiment, the AAV is administered with a diluent, if necessary to obtain the desired therapeutic dose.

In another embodiment, an AAV described herein is formulated to a concentration of 2.0×10¹³ vg/mL in 20 mM Tris pH 8.0, 1 mM MgCl₂, 200 mM NaCl, and 0.005% poloxamer 188.

In one embodiment, the AAV vector is formulated at a concentration from about 1×10¹² vg/ml to 1×10¹³ vg/ml; from about 10 mM to 30 mM Tris; from about 150 mM to 300 mM NaCl; from about 0.5 mM to 3.0 mM MgCl₂·6H₂O; and from about 0.002% (w/v) to 0.02% (w/v) poloxamer 188, such as Pluronic® F-68, wherein the formulation has a pH of from 7.8 to 8:2

In one embodiment, the AAV vector is formulated at a concentration of from 5.0×10¹² to 1×10¹³ GC/ml; about 20 mM Tris; about 200 mM NaCl; about 1.0 mM MgCl₂·6H₂O; and about 0.01% (w/v) poloxamer 188, such as Pluronic® F-68; wherein the formulation has a pH of about 8.0.

In another embodiment, the AAV vector described herein is formulated to comprise a recombinant AAV FVIII-encoding virus, a buffering agent, an isotonicity agent, a bulking agent and a surfactant. In some embodiments, the formulations of the present invention comprises any of the AAV-FVIII-QQ viruses described herein, p-100 ATGB or any of the other herein described vectors and/or are stable during storage at ≤65° C. for at least 2 weeks.

In a one embodiment of the present invention, the formulation of the present invention comprises any of the AAV-FVIII-QQ described herein formulated in a liquid solution that comprises about 1.42 mg/m of sodium phosphate, dibasic, about 1.38 mg/ml of sodium phosphate, monobasic monohydrate, about 8.18 mg/ml sodium chloride, about 20 mg/ml mannitol and about 2 mg/ml poloxamer 188. In one embodiment, the pH of the formulation is 7.4, In one embodiment, the concentration of recombinant AAV virus in the above described formulation was 2E13 vg/ml.

In one embodiment, the concentration of recombinant AAV virus in the above described formulation was 2E13 vg/ml.

Additional exemplary compositions/compositions comprising rAAV are described in International Patent Application No. PCT/US2022/0137279, U.S. patent application Ser. No. 17/725,086, and U.S. Pat. No. 10,512,675 the content of which is incorporated herein by reference in its entirety.

The rAAV vectors containing a codon-optimized nucleic acid encoding a human FVIII polypeptide as disclosed herein, for use in the methods of administration as disclosed herein may be used in combination with one or more other therapeutic, prophylactic, research or diagnostic agents. By “in combination with,” it is not intended to imply that the agents must be administered at the same time and/or formulated for delivery together, although these methods of delivery are within the scope of the present invention. Compositions can be administered concurrently with, prior to, or subsequent to, one or more other desired therapeutics or medical procedures. In some embodiments, the delivery of one treatment (e.g., gene therapy vectors) is still occurring when the delivery of the second (e.g., one or more therapeutic) begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. The composition described herein and the at least one additional therapy can be administered simultaneously, in the same or in separate compositions, or sequentially. For sequential administration, the gene therapy vectors described herein can be administered first, and the one or more therapeutic can be administered second, or the order of administration can be reversed. The gene therapy vectors and the one or more therapeutic can be administered during periods of active disorder, or during a period of remission or less active disease. The gene therapy vectors can be administered before another treatment, concurrently with the treatment, post-treatment, or during remission of the disorder.

When administered in combination, the rAAV vectors as disclosed herein for use in the methods of administration as disclosed herein and the one or more therapeutic (e.g., second or third therapeutic), or all, can be administered in an amount or dose that is higher, lower or the same as the amount or dosage of each used individually, e.g., as a monotherapy. In certain embodiments, the administered amount or dosage of a rAAV vector as disclosed herein for use in the methods of administration as disclosed herein and the one or more therapeutic (e.g., second or third agent), or all, is lower (e.g., at least 20%, at least 30%, at least 40%, or at least 50%) than the amount or dosage of each used individually. In other embodiments, the amount or dosage of the rAAV vector as disclosed herein for use in the methods of administration as disclosed herein and the one or more therapeutic (e.g., second or third agent), or all, that results in a desired effect (e.g., treatment of a cardiovascular disease or heart disease) is lower (e.g., at least 20%, at least 30%, at least 40%, or at least 50% lower) than the amount or dosage of each individually required to achieve the same therapeutic effect.

In some embodiments, the methods of administration of a rAAV vector as disclosed herein can deliver a rAVV vector disclosed herein alone, or in combination with an additional agent, for example, an immune modulator as disclosed herein.

### Definitions

The following terms are used in the description herein and the appended claims:

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Furthermore, the term “about,” as used herein when referring to a measurable value such as an amount of the length of a polynucleotide or polypeptide sequence, dose, time, temperature, and the like, is meant to encompass variations off ±20%, ±10%, ±5%, ±1%, ±0.5%, or even ±0.1% of the specified amount.

Also as used herein, “and/or” refers to and encompasses any and all possible combinations of one or more of the associated listed items, as well as the lack of combinations when interpreted in the alternative (“or”).

As used herein, the transitional phrase “consisting essentially of’ means that the scope of a claim is to be interpreted to encompass the specified materials or steps recited in the claim, “and those that do not materially affect the basic and novel characteristic(s)” of the claimed invention. See, In re Herz, 537 F.2d 549, 551-52, 190 USPQ 461,463 (CCPA 1976) (emphasis in the original); see also MPEP § 2111.03. Thus, the term “consisting essentially of’ when used in a claim of this invention is not intended to be interpreted to be equivalent to “comprising.” Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

To illustrate further, if, for example, the specification indicates that a particular amino acid can be selected from A, G, I, L and/or V, this language also indicates that the amino acid can be selected from any subset of these amino acid(s) for example A, G, I or L; A, G, I or V; A or G; only L; etc. as if each such subcombination is expressly set forth herein. Moreover, such language also indicates that one or more of the specified amino acids can be disclaimed (e.g., by negative proviso). For example, in particular embodiments the amino acid is not A, G or I; is not A; is not G or V; etc. as if each such possible disclaimer is expressly set forth herein.

The term “parvovirus” as used herein encompasses the family Parvoviridae, including autonomously replicating parvoviruses and dependoviruses. The autonomous parvoviruses include members of the genera Parvovirus, Erythrovirus, Densovirus, Iteravirus, and Contravirus. Exemplary autonomous parvoviruses include, but are not limited to, minute virus of mouse, bovine parvovirus, canine parvovirus, chicken parvovirus, feline panleukopenia virus, feline parvovirus, goose parvovirus, H1 parvovirus, Muscovy duck parvovirus, B19 virus, and any other autonomous parvovirus now known or later discovered. Other autonomous parvoviruses are known to those skilled in the art. See, e.g., BERNARD N. FIELDS et al., VIROLOGY, volume 2, chapter 69 (4th ed., Lippincott-Raven Publishers).

As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, avian AAV, bovine AAV, canine AAV, equine AAV, ovine AAV, any AAV disclosed in Table 2 herein, and any other AAV now known or later discovered. See, e.g., BERNARD N. FIELDS et al., VIROLOGY, volume 2, chapter 69 (4th ed., Lippincott-Raven Publishers). A number of relatively new AAV serotypes and clades have been identified (see, e.g., Gao et al., (2004) J. Virology 78:6381-6388; Moris et al., (2004) Virology 33-:375-383); and also Table 1 as disclosed in U.S. Provisional Application 62,937,556, filed on Nov. 19, 2019 and Table 1 in International Applications WO2020/102645, and WO2020/102667, each of which is incorporated herein in their entirety.

The genomic sequences of various serotypes of AAV and the autonomous parvoviruses, as well as the sequences of the native inverted terminal repeats (ITRs), Rep proteins, and capsid subunits are known in the art. Such sequences may be found in the literature or in public databases such as GenBank. See, e.g., GenBank Accession Numbers NC_002077, NC_001401, NC_001729, NC_001863, NC_001829, NC_001862, NC_000883, NC_001701, NC_001510, NC_006152, NC_006261, AF063497, U89790, AF043303, AF028705, AF028704, J02275, J01901, J02275, X01457, AF288061, AH009962, AY028226, AY028223, NC_001358, NC_001540, AF513851, AF513852, AY530579; the disclosures of which are incorporated by reference herein for teaching parvovirus and AAV nucleic acid and amino acid sequences. See also, e.g., Srivistava et al., (1983) J Virology 45:555; Chiarini et al., (1998) J. Virology 71:6823; Chiarini et al., (1999) J. Virology 73:1309; Bantel-Schaal et al., (1999) J. Virology 73:939; Xiao et al., (1999) J. Virology 73:3994; Muramatsu et al., (1996) Virology 221:208; Shade et al., (1986) J. Viral. 58:921; Gao et al., (2002) Proc. Nat. Acad. Sci. USA 99:11854; Morris et al., (2004) Virology 33-:375-383; international patent publications WO 00/28061, WO 99/61601, WO 98/11244; and U.S. Pat. No. 6,156,303; the disclosures of which are incorporated by reference herein for teaching parvovirus and AAV nucleic acid and amino acid sequences. See also Table 1 and Table 5 disclosed in 62/937,556, filed on Nov. 19, 2019 or Table 1 as disclosed in International Applications WO2020/102645, and WO2020/102667, each of which is incorporated herein in their entirety. The capsid structures of autonomous parvoviruses and AAV are described in more detail in BERNARD N. FIELDS et al., VIROLOGY, volume 2, chapters 69 & 70 (4th ed., Lippincott-Raven Publishers). See also, description of the crystal structure of AAV2 (Xie et al., (2002) Proc. Nat. Acad. Sci. 99:10405-10), AAV4 (Padron et al., (2005) J. Viral. 79: 5047-58), AAV5 (Walters et al., (2004) J. Viral. 78: 3361-71) and CPV (Xie et al., (1996) J. Mal. Biol. 6:497-520 and Tsao et al., (1991) Science 251: 1456-64).

The term “tropism” as used herein refers to preferential entry of the virus into certain cells or tissues, optionally followed by expression (e.g., transcription and, optionally, translation) of a sequence(s) carried by the viral genome in the cell, e.g., for a recombinant virus, expression of a heterologous nucleic acid(s) of interest.

As used here, “systemic tropism” and “systemic transduction” (and equivalent terms) indicate that the virus capsid or virus vector of the invention exhibits tropism for and/or transduces tissues throughout the body (e.g., brain, lung, skeletal muscle, heart, liver, kidney and/or pancreas).

As used herein, “selective tropism” or “specific tropism” means delivery of virus vectors to and/or specific transduction of certain target cells and/or certain tissues.

Unless indicated otherwise, “efficient transduction” or “efficient tropism,” or similar terms, can be determined by reference to a suitable control (e.g., at least about 50%, 60%, 70%, 80%, 85%, 90%, 95%, 100%, 125%, 150%, 175%, 200%, 250%, 300%, 350%, 400%, 500% or more of the transduction or tropism, respectively, of the control). In particular embodiments, the virus vector efficiently transduces or has efficient tropism for liver cells and muscle cells. Suitable controls will depend on a variety of factors including the desired tropism and/or transduction profile.

Similarly, it can be determined if a virus “does not efficiently transduce” or “does not have efficient tropism” for a target tissue, or similar terms, by reference to a suitable control. In particular embodiments, the virus vector does not efficiently transduce (i.e., has does not have efficient tropism) for kidney, gonads and/or germ cells. In particular embodiments, transduction (e.g., undesirable transduction) of tissue(s) (e.g., kidney) is 20% or less, 10% or less, 5% or less, 1% or less, 0.1% or less of the level of transduction of the desired target tissue(s) (e.g., liver, skeletal muscle, diaphragm muscle, cardiac muscle and/or cells of the central nervous system).

As used herein, the term “polypeptide” encompasses both peptides and proteins, unless indicated otherwise.

A “polynucleotide” is a sequence of nucleotide bases, and may be RNA, DNA or DNA-RNA hybrid sequences (including both naturally occurring and non-naturally occurring nucleotides), but in representative embodiments are either single or double stranded DNA sequences.

The terms “heterologous nucleotide sequence” and “heterologous nucleic acid molecule” are used interchangeably herein and refer to a nucleic acid sequence that is not naturally occurring in the virus. Generally, the heterologous nucleic acid molecule or heterologous nucleotide sequence comprises an open reading frame that encodes a polypeptide and/or nontranslated RNA of interest (e.g., for delivery to a cell and/or subject).

A “chimeric nucleic acid” comprises two or more nucleic acid sequences covalently linked together to encode a fusion polypeptide. The nucleic acids may be DNA, RNA, or a hybrid thereof.

The term “fusion polypeptide” comprises two or more polypeptides covalently linked together, typically by peptide bonding.

As used herein, an “isolated” polynucleotide (e.g., an “isolated DNA” or an “isolated RNA”) means a polynucleotide at least partially separated from at least some of the other components of the naturally occurring organism or virus, for example; the cell or viral structural components or other polypeptides or nucleic acids commonly found associated with the polynucleotide. In representative embodiments an “isolated” nucleotide is enriched by at least about 10-fold, 100′-fold, 1000-fold, 10,000-fold or more as compared with the starting material.

Likewise, an “isolated” polypeptide means a polypeptide that is at least partially separated from at least some of the other components of the naturally occurring organism or virus, for example, the cell or viral structural components or other polypeptides or nucleic acids commonly found associated with the polypeptide. In representative embodiments an “isolated” polypeptide is enriched by at least about 10-fold, 100-fold, 1000-fold, 10,000-fold or more as compared with the starting material.

An “isolated cell” refers to a cell that is separated from other components with which it is normally associated in its natural state. For example, an isolated cell can be a cell in culture medium and/or a cell in a pharmaceutically acceptable carrier of this invention. Thus, an isolated cell can be delivered to and/or introduced into a subject. In some embodiments, an isolated cell can be a cell that is removed from a subject and manipulated as described herein ex vivo and then returned to the subject.

A population of virions can be generated by any of the methods described herein. In one embodiment, the population is at least 101 virions. In one embodiment, the population is at least 102 virions, at least 103, virions, at least 104 virions, at least 105 virions, at least 106 virions, at least 107 virions, at least 108 virions, at least 109 virions, at least 1010 virions, at least 1011 virions, at least 1012 virions, at least 1013 virions, at least 1014 virions, at least 1015 virions, at least 1016 virions, or at least 1017 virions. A population of virions can be heterogeneous or can be homogeneous (e.g., substantially homogeneous or completely homogeneous).

A “substantially homogeneous population” as the term is used herein, refers to a population of virions that are mostly identical, with few to no contaminant virions (those that are not identical) therein. A substantially homogeneous population is at least 90% of identical virions (e.g., the desired virion), and can be at least 91%, at least 92%, at least 93%, at least 94%, at least 95%, at least 96%, at least 97%, at least 98%, at least 99%, at least 99.5%, at least 99.9% of identical virions.

A population of virions that is completely homogeneous contains only identical virions.

As used herein, by “isolate” or “purify” (or grammatical equivalents) a virus vector or virus particle or population of virus particles, it is meant that the virus vector or virus particle or population of virus particles is at least partially separated from at least some of the other components in the starting material. In representative embodiments an “isolated” or “purified” virus vector or virus particle or population of virus particles is enriched by at least about 10-fold, 100-fold, 1000-fold, 10,000-fold or more as compared with the starting material.

Unless indicated otherwise, “efficient transduction” or “efficient tropism,” or similar terms, can be determined by reference to a suitable control (e.g., at least about 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 250%, 300%, 350%, 400%, 500% or more of the transduction or tropism, respectively, of the control). In particular embodiments, the virus vector efficiently transduces or has efficient tropism for neuronal cells and cardiomyocytes. Suitable controls will depend on a variety of factors including the desired tropism and/or transduction profile.

A “therapeutic polypeptide” is a polypeptide that can alleviate, reduce, prevent, delay and/or stabilize symptoms that result from an absence or defect in a protein in a cell or subject and/or is a polypeptide that otherwise confers a benefit to a subject, e.g., enzyme replacement to reduce or eliminate symptoms of a disease, or improvement in transplant survivability or induction of an immune response.

The terms “heterologous nucleotide sequence” and “heterologous nucleic acid molecule” are used interchangeably herein and refer to a nucleic acid sequence that is not naturally occurring in the virus. Generally, the heterologous nucleic acid molecule or heterologous nucleotide sequence comprises an open reading frame that encodes a polypeptide and/or nontranslated RNA of interest (e.g., for delivery to a cell and/or subject), for example the FVIII polypeptide.

As used herein, the terms “virus vector,” “vector” or “gene delivery vector” refer to a virus (e.g., AAV) particle that functions as a nucleic acid delivery vehicle, and which comprises the vector genome (e.g., viral DNA [vDNA]) packaged within a virion. Alternatively, in some contexts, the term “vector” may be used to refer to the vector genome/vDNA alone.

An “rAAV vector genome” or “rAAV genome” is an AAV genome (i.e., vDNA) that comprises one or more heterologous nucleic acid sequences. rAAV vectors generally require only the inverted terminal repeat(s) (TR(s)) in cis to generate virus. All other viral sequences are dispensable and may be supplied in trans (Muzyczka, (1992) Curr. Topics Microbial. Immunol. 158:97). Typically, the rAAV vector genome will only retain the one or more TR sequence so as to maximize the size of the transgene that can be efficiently packaged by the vector. The structural and non-structural protein coding sequences may be provided in trans (e.g., from a vector, such as a plasmid, or by stably integrating the sequences into a packaging cell). In embodiments of the invention the rAAV vector genome comprises at least one ITR sequence (e.g., AAV TR sequence), optionally two ITRs (e.g., two AAV TRs), which typically will be at the 5′ and 3′ ends of the vector genome and flank the heterologous nucleic acid, but need not be contiguous thereto. The TRs can be the same or different from each other.

The term “terminal repeat” or “TR” includes any viral terminal repeat or synthetic sequence that forms a hairpin structure and functions as an inverted terminal repeat (i.e., an ITR that mediates the desired functions such as replication, virus packaging, integration and/or provirus rescue, and the like). The TR can be an AAV TR or a non-AAV TR. For example, a non-AAV TR sequence such as those of other parvoviruses (e.g., canine parvovirus (CPV), mouse parvovirus (MVM), human parvovirus B-19) or any other suitable virus sequence (e.g., the SV40 hairpin that serves as the origin of SV40 replication) can be used as a TR, which can further be modified by truncation, substitution, deletion, insertion and/or addition. Further, the TR can be partially or completely synthetic, such as the “double-D sequence” as described in U.S. Pat. No. 5,478,745 to Samulski et al.

An “AAV terminal repeat” or “AAV TR,” including an “AAV inverted terminal repeat” or “AAV ITR” may be from any AAV, including but not limited to serotypes 1, 2, 3, 4, 5, 6, 7, 8, 9, 10, 11 or 12 or any other AAV now known or later discovered. An AAV terminal repeat need not have the native terminal repeat sequence (e.g., a native AAV TR or AAV ITR sequence may be altered by insertion, deletion, truncation and/or missense mutations), as long as the terminal repeat mediates the desired functions, e.g., replication, virus packaging, integration, and/or provirus rescue, and the like.

AAV proteins VP1, VP2 and VP3 are capsid proteins that interact together to form an AAV capsid of an icosahedral symmetry. VP1.5 is an AAV capsid protein described in US Publication No. 2014/0037585.

The virus vectors of the invention can further be “targeted” virus vectors (e.g., having a directed tropism) and/or a “hybrid” parvovirus (i.e., in which the viral TRs and viral capsid are from different parvoviruses) as described in international patent publication WO 00/28004 and Chao et al., (2000) Molecular Therapy 2:619.

The virus vectors of the invention can further be duplexed parvovirus particles as described in international patent publication WO 01/92551 (the disclosure of which is incorporated herein by reference in its entirety). Thus, in some embodiments, double stranded (duplex) genomes can be packaged into the virus capsids of the invention.

Further, the viral capsid or genomic elements can contain other modifications, including insertions, deletions and/or substitutions.

A “chimeric’ capsid protein as used herein means an AAV capsid protein (e.g., any one or more of VP1, VP2 or VP3) that has been modified by substitutions in one or more (e.g., 2, 3, 4, 5, 6, 7, 8, 9, 10, etc.) amino acid residues in the amino acid sequence of the capsid protein relative to wild type, as well as insertions and/or deletions of one or more (e.g., 2, 3, 4, 5, 6, 7, 8, 9, 10, etc.) amino acid residues in the amino acid sequence relative to wild type. In some embodiments, complete or partial domains, functional regions, epitopes, etc., from one AAV serotype can replace the corresponding wild type domain, functional region, epitope, etc. of a different AAV serotype, in any combination, to produce a chimeric capsid protein of this invention. Production of a chimeric capsid protein can be carried out according to protocols well known in the art and a significant number of chimeric capsid proteins are described in the literature as well as herein that can be included in the capsid of this invention.

As used herein, the term “haploid AAV” shall mean that AAV as described in International Application WO2018/170310, or US Application US2018/037149, which are incorporated herein in their entirety by reference. In some embodiments, a population of virions is a haploid AAV population where a virion particle can be constructed wherein at least one viral protein from the group consisting of AAV capsid proteins, VP1, VP2 and VP3, is different from at least one of the other viral proteins, required to form the virion particle capable of encapsulating an AAV genome. For each viral protein present (VP1, VP2, and/or VP3), that protein is the same type (e.g., all AAV2 VP1). In one instance, at least one of the viral proteins is a chimeric viral protein and at least one of the other two viral proteins is not a chimeric. In one embodiment VP1 and VP2 are chimeric and only VP3 is non-chimeric. For example, only the viral particle composed of VP1/VP2 from the chimeric AAV2/8 (the N-terminus of AAV2 and the C-terminus of AAV8) paired with only VP3 from AAV2; or only the chimeric VP1/VP2 28m-2P3 (the N-terminal from AAV8 and the C-terminal from AAV2 without mutation of VP3 start codon) paired with only VP3 from AAV2. In another embodiment only VP3 is chimeric and VP1 and VP2 are non-chimeric. In another embodiment at least one of the viral proteins is from a completely different serotype. For example, only the chimeric VP1/VP2 28m-2P3 paired with VP3 from only AAV3. In another example, no chimeric is present.

The term a “hybrid” AAV vector or parvovirus refers to a rAAV vector where the viral TRs or ITRs and viral capsid are from different parvoviruses. Hybrid vectors are described in international patent publication WO 00/28004 and Chao et al., (2000) Molecular Therapy 2:619. For example, a hybrid AAV vector typically comprises the adenovirus 5′ and 3′ cis ITR sequences sufficient for adenovirus replication and packaging (i.e., the adenovirus terminal repeats and PAC sequence).

The term “polyploid AAV” refers to a AAV vector which is composed of capsids from two or more AAV serotypes, e.g., and can take advantages from individual serotypes for higher transduction but not in certain embodiments eliminate the tropism from the parents.

As used herein, the term “amino acid” encompasses any naturally occurring amino acid, modified forms thereof, and synthetic amino acids. Naturally occurring, levorotatory (L-) amino acids are disclosed in Table 2 of US Publication 2018/0371496, which is incorporated herein in its entirety. Alternatively, the amino acid can be a modified amino acid residue (nonlimiting examples are shown in Table 4 of US Publication of US Publication 2018/0371496) and/or can be an amino acid that is modified by post-translation modification (e.g., acetylation, amidation, formylation, hydroxylation, methylation, phosphorylation or sulfatation). Further, the non-naturally occurring amino acid can be an “unnatural” amino acid as described by Wang et al., Annu Rev Biophys Biomol Struct. 35:225-49 (2006). These unnatural amino acids can advantageously be used to chemically link molecules of interest to the AAV capsid protein.

To illustrate further, if, for example, the specification indicates that a particular amino acid can be selected from A, G, I, L and/or V, this language also indicates that the amino acid can be selected from any subset of these amino acid(s) for example A, G, I or L; A, G, I or V; A or G; only L; etc. as if each such subcombination is expressly set forth herein. Moreover, such language also indicates that one or more of the specified amino acids can be disclaimed (e.g., by negative proviso). For example, in particular embodiments the amino acid is not A, G or I; is not A; is not G or V; etc. as if each such possible disclaimer is expressly set forth herein.

As used herein, the phrase “promoter” refers to a region of DNA that generally is located upstream of a nucleic acid sequence to be transcribed that is needed for transcription to occur, i.e., which initiates transcription. Promoters permit the proper activation or repression of transcription of a coding sequence under their control. A promoter typically contains specific sequences that are recognized and bound by plurality of TFs. TFs bind to the promoter sequences and result in the recruitment of RNA polymerase, an enzyme that synthesizes RNA from the coding region of the gene. A great many promoters are known in the art.

The term “synthetic promoter” as used herein relates to a promoter that does not occur in nature. Parts of the synthetic promoter may be naturally occurring (e.g., the minimal promoter), but the synthetic promoter as a complete entity is not naturally occurring.

As used herein, “minimal promoter” (also known as the “core promoter”) refers to a short DNA segment which is inactive or largely inactive by itself, but can mediate transcription when combined with other transcription regulatory elements. Minimum promoter sequence can be derived from various different sources, including prokaryotic and eukaryotic genes. Examples of minimal promoters are discussed above, and include the dopamine beta-hydroxylase gene minimum promoter, cytomegalovirus (CMV) immediate early gene minimum promoter (CMV-MP), and the herpes thymidine kinase minimal promoter (MinTK). A minimal promoter typically comprises the transcription start site (TSS) and elements directly upstream, a binding site for RNA polymerase II, and general transcription factor binding sites (often a TATA box).

As used herein, “proximal promoter” relates to the minimal promoter plus the proximal sequence upstream of the gene that tends to contain primary regulatory elements. It often extends approximately 250 base pairs upstream of the TSS, and includes specific TFBS. The proximal promoter can be a naturally occurring liver-specific proximal promoter. However, the proximal promoter can be synthetic.

A “functional variant” of a promoter or other nucleic acid sequence in the context of the present invention is a variant of a reference sequence that retains the ability to function in the same way as the reference sequence, e.g., as a liver-specific promoter. Alternative terms for such functional variants include “biological equivalents” or “equivalents”. As the term “functional variant” is used herein in reference to a polypeptide, refers to a polypeptide resulting from one or more amino acid substitution, deletion or insertions, which retains a substantial amount of one or more biological activities (e.g., activity involved in treating hemophelia A) of the reference polypeptide, e.g., by at least about 75%, 80%, 85%, 90%, 91%, 92%, 93%, 94%, 95%, 96%, 97%, 98%, 99%, 100% or more, as determined by various available in vitro and/or in vivo assays. One example of the activity of the Factor VIII polypeptide described herein is its activity as a Factor IXa cofactor.

The term “CpG island” refers to a region within a polynucleotide having a statistically elevated density of CpG dinucleotides. For information on methods for identifying CpG islands, see Gardiner-Garden M. et al., J Mol Biol., 196(2):261-82 (1987), the contents of which are incorporated herein by reference in its entirety.

An “exogenous” molecule is a molecule that is introduced into a subject (e.g., by introducing into cells of the subject) by one or more genetic, biochemical or other methods. An exogenous molecule can comprise, for example, a functioning version of an absent or malfunctioning endogenous molecule. By contrast, an “endogenous” molecule is one that is present naturally in subject or a cell.

The term “expression cassette” as used herein, refers minimally to a nucleic acid that encodes a polypeptide operatively linked to a promoter. In the expression cassette, the coding region may further be operatively linked to other elements such as a polyA sequence and other regulatory elements such as 5′ UTR, enhancers, etc.

The terms “liver-specific” or “liver-specific expression” when in reference to a promoter refers to the ability of promoter to enhance or drive expression of a gene in the liver (or in liver-derived cells) in a preferential or predominant manner as compared to other tissues (e.g., spleen, muscle, heart, lung, and brain). Expression of the gene can be in the form of mRNA or protein. In some embodiments, liver-specific expression is such that there is negligible expression in other (i.e., non-liver) tissues or cells, i.e., expression is highly liver-specific. In some embodiments, while a liver-specific promoter drives expression preferentially in the liver, it can also drive expression of the gene in another tissue of interest at a lower level, e.g., muscle.

The skilled person can thus easily determine whether any variant of the liver-specific promoter recited above remains functional (i.e., it is a functional variant as defined above). For example, any given promoter to be assessed can be operably linked to a minimal promoter (e.g., positioned upstream of CMV-MP) and the ability of the promoter to drive liver-specific expression of a gene (typically a reporter gene) is measured. Similarly, the ability of a promoter to drive liver-specific expression can be readily assessed by the skilled person (e.g., as described in the examples below). Expression levels of a gene driven by a variant of a reference promoter can be compared to the expression levels driven by the reference sequence. In some embodiments, where liver-specific expression levels driven by a variant promoter are at least 50%, at least 60%, at least 70%, at least 80%, at least 90%, or at least 100% of the expression levels driven by the reference promoter, it can be said that the variant remains functional. Suitable nucleic acid constructs and reporter assays to assess liver-specific expression enhancement can easily be constructed, and the examples set out below give suitable methodologies.

Liver-specificity can be identified wherein the expression of a gene (e.g., a therapeutic or reporter gene) occurs preferentially or predominantly in liver-derived cells. Preferential or predominant expression can be defined, for example, where the level of expression is significantly greater in liver-derived cells than in other types of cells (i.e., non-liver-derived cells). For example, expression in liver-derived cells is suitably at least 5-fold higher than non-liver cells, preferably at least 10-fold higher than non-liver cells, and it may be 50-fold higher or more in some cases. For convenience, liver-specific expression can suitably be demonstrated via a comparison of expression levels in a hepatic cell line (e.g., liver-derived cell line such as Huh7 and/or HepG2 cells) or liver primary cells, compared with expression levels in a kidney-derived cell line (e.g., HEK-293), a cervical tissue-derived cell line (e.g., HeLa) and/or a lung-derived cell line (e.g., A549).

The synthetic liver-specific promoters of the present invention are preferably suitable for promoting expression in the liver of a subject, e.g., driving liver-specific expression of a transgene, preferably a therapeutic transgene.

Preferred synthetic liver-specific promoters of the present invention are suitable for promoting liver-specific transgene expression and have an activity in liver cells which is at least 15%, 20%, 30%, 40%, 50%, 60%, 70%, 80%, 90%, 100%, 125%, 150%, 175%, 200%, 250%, 300%, 350% or 400% of the activity of the TBG promoter (see, e.g., SEQ ID NO: 435 as disclosed in International Application WO2021102107).

The synthetic liver-specific promoters of the present invention are preferably suitable for promoting liver-specific expression at a level at least 1.5-fold greater than a CMV-IE promoter (see, e.g., SEQ ID NO: 433 as disclosed in International Application WO2021102107) in liver-derived cells, preferably at least 2-fold greater than a CMV promoter in liver-derived cells (e.g., HEK-293, HeLa, and/or A549 cells).

The terms “identity” and “identical” and the like refer to the sequence similarity between two polymeric molecules, e.g., between two nucleic acid molecules, such as between two DNA molecules. Sequence alignments and determination of sequence identity can be done, e.g., using the Basic Local Alignment Search Tool (BLAST) originally described by Altschul et al. 1990 (J Mol Biol 215: 403-10), such as the “Blast 2 sequences” algorithm described by Tatusova and Madden 1999 (FEMS Microbiol Lett 174: 247-250).

Methods for aligning sequences for comparison are well-known in the art. Various programs and alignment algorithms are described in, for example: Smith and Waterman (1981) Adv. Appl. Math. 2:482; Needleman and Wunsch (1970) J. Mol. Biol. 48:443; Pearson and Lipman (1988) Proc. Natl. Acad. Sci. U.S.A. 85:2444; Higgins and Sharp (1988) Gene 73:237-44; Higgins and Sharp (1989) CABIOS 5:151-3; Corpet et al. (1988) Nucleic Acids Res. 16:10881-90; Huang et al. (1992) Comp. Appl. Biosci. 8:155-65; Pearson et al. (1994) Methods Mol. Biol. 24:307-31; Tatiana et al. (1999) FEMS Microbiol. Lett. 174:247-50. A detailed consideration of sequence alignment methods and homology calculations can be found in, e.g., Altschul et al. (1990) J. Mol. Biol. 215:403-10.

The National Center for Biotechnology Information (NCBI) Basic Local Alignment Search Tool (BLAST™; Altschul et al. (1990)) is available from several sources, including the National Center for Biotechnology Information (Bethesda, MD), and on the internet, for use in connection with several sequence analysis programs. A description of how to determine sequence identity using this program is available on the internet under the “help” section for BLAST™. For comparisons of nucleic acid sequences, the “Blast 2 sequences” function of the BLAST™ (Blastn; Align Sequence Nucleotide BLAST) program may be employed using the default parameters. Nucleic acid sequences with even greater similarity to the reference sequences will show increasing percentage identity when assessed by this method. Typically, the percentage sequence identity is calculated over the entire length of the sequence.

For example, a global optimal alignment is suitably found by the Needleman-Wunsch algorithm with the following scoring parameters: Match score: +2, Mismatch score: −3; Gap penalties: gap open 5, gap extension 2. The percentage identity of the resulting optimal global alignment is suitably calculated by the ratio of the number of aligned bases to the total length of the alignment, where the alignment length includes both matches and mismatches, multiplied by 100.

The term “synthetic” as used herein means a nucleic acid molecule that does not occur in nature. Synthetic nucleic acid expression constructs of the present invention are produced artificially, typically by recombinant technologies. Such synthetic nucleic acids may contain naturally occurring sequences (e.g., promoter, enhancer, intron, and other such regulatory sequences), but these are present in a non-naturally occurring context. For example, a synthetic gene (or portion of a gene) typically contains one or more nucleic acid sequences that are not contiguous in nature (chimeric sequences), and/or may encompass substitutions, insertions, and deletions and combinations thereof.

A “spacer sequence” or “spacer” as used herein is a nucleic acid sequence that separates two functional nucleic acid sequences. It can have essentially any sequence, provided it does not prevent the functional nucleic acid sequence (e.g., cis-regulatory element) from functioning as desired (e.g., this could happen if it includes a silencer sequence, prevents binding of the desired transcription factor, or suchlike). Typically, it is non-functional, as in it is present only to space adjacent functional nucleic acid sequences from one another.

The term “pharmaceutically acceptable” as used herein is consistent with the art and means compatible with the other ingredients of the pharmaceutical composition and not deleterious to the recipient thereof.

By the terms “treat,” “treating” or “treatment of’ (and grammatical variations thereof) it is meant that the severity of the subject's condition is reduced, at least partially improved or stabilized and/or that some alleviation, mitigation, decrease or stabilization in at least one clinical symptom is achieved and/or there is a delay in the progression of the disease or disorder.

The terms “prevent,” “preventing” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset is substantially less than what would occur in the absence of the present invention.

A “treatment effective” amount as used herein is an amount that is sufficient to provide some improvement or benefit to the subject. Alternatively stated, a “treatment effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some preventative benefit is provided to the subject.

The phrase a “therapeutically effective amount” and like phrases mean a dose or plasma concentration in a subject that provides the desired specific pharmacological effect, e.g., to express a therapeutic gene in the liver, and secretion into the plasma. It is emphasized that a therapeutically effective amount may not always be effective in treating the conditions described herein, even though such dosage is deemed to be a therapeutically effective amount by those of skill in the art. The therapeutically effective amount may vary based on the route of administration and dosage form, the age and weight of the subject, and/or the disease or condition being treated.

The terms “individual,” “subject,” and “patient” are used interchangeably, and refer to any individual subject with a disease or condition in need of treatment. For the purposes of the present disclosure, the subject may be a primate, preferably a human, or another mammal, such as a dog, cat, horse, pig, goat, or bovine, and the like.

Additional patents incorporated for reference herein that are related to, disclose or describe an AAV or an aspect of an AAV, including the DNA vector that includes the gene of interest to be expressed are: U.S. Pat. Nos. 6,491,907; 7,229,823; 7,790,154; 7,201,898; 7,071,172; 7,892,809; 7,867,484; 8,889,641; 9,169,494; 9,169,492; 9,441,206; 9,409,953; and, 9,447,433; 9,592,247; and, 9,737,618.

The present invention can further be described in any one of the following numbered paragraphs:
1. A codon-optimized nucleic acid encoding a human Factor VIII (FVIII) polypeptide, wherein the encoded FVIII polypeptide lacks the B domain, and further comprises an amino acid substitution of Glutamine for Arginine at position 355 (R355Q) and of Glutamine for Arginine at position 581 (R581Q), wherein the nucleic acid comprises the nucleotide sequence set forth in any one of SEQ ID NOs 1, 2, 4, 5, 7-9, 11-15 or 18, or a nucleic acid having at least 90%, 91%, 92%, 93%, 94%, 95%, 96%, 97%, or 98%, or 99% sequence identity thereto.
2. The codon-optimized nucleic acid of paragraph 1, wherein the nucleic acid comprises the nucleotide sequence set forth in any one of SEQ ID NOs 4, 5, 7, 12-15 or 18, or a nucleic acid having at least 90%, 91%, 92%, 93%, 94%, 95%, 96%, 97%, or 98%, or 99% sequence identity thereto.
3. The codon-optimized nucleic acid of any of the proceeding paragraphs, wherein the nucleic acid comprises the nucleotide sequence set forth in any one of SEQ ID NOs 4, 5, 13, or 15, or a nucleic acid having at least 90%, 91%, 92%, 93%, 94%, 95%, 96%, 97%, or 98%, or 99% sequence identity thereto.
4. The codon-optimized nucleic acid of any of the proceeding paragraphs, wherein the nucleic acid comprises the nucleotide sequence set forth in any one of SEQ ID NOs 4 or 5, or a nucleic acid having at least 90%, 91%, 92%, 93%, 94%, 95%, 96%, 97%, or 98%, or 99% sequence identity thereto.
5. The codon-optimized nucleic acid of any of the proceeding paragraphs, wherein the human FVIII polypeptide is a functional variant of the human FVIII polypeptide with the amino acid sequence shown in SEQ ID NO: 19.
6. The codon-optimized nucleic acid of any of the proceeding paragraphs, wherein the functional variant has at least 60%, or 70%, or 80%, 85% or 90% or 95%, or 98%, or 99% sequence identity to the amino acid sequence shown in SEQ ID NO: 19.
7. The codon-optimized nucleic acid of any of the proceeding paragraphs, wherein the B domain of the encoded FVIII polypeptide has been replaced by a peptide linker.
8. The codon-optimized nucleic acid of any of the proceeding paragraphs, wherein the encoded FVIII polypeptide lacks both the amino acid substitution of Glutamine for Arginine at position 355 (R355Q) and of Glutamine for Arginine at position 581 (R581Q).
9. The codon-optimized nucleic acid of any of the proceeding paragraphs, wherein the encoded FVIII polypeptide lacks the amino acid substitution of Glutamine for Arginine at position 355 (R355Q).
10. The codon-optimized nucleic acid of any of the proceeding paragraphs, wherein the encoded FVIII polypeptide lacks the amino acid substitution of Glutamine for Arginine at position 581 (R581Q).
11. The codon-optimized nucleic acid of any of the proceeding paragraphs, that is comprised within a nucleic acid construct that further comprises viral sequence elements that facilitate integration and expression.
12. An expression cassette containing the codon-optimized nucleic acid of any of the proceeding paragraphs, operably linked to a constitutive promoter.
13. The expression cassette of any of the proceeding paragraphs, wherein the constitutive promoter is a TTR promoter.
14. The expression cassette of any of the proceeding paragraphs, wherein the TTR promoter comprises a nucleic acid sequence of SEQ ID NO: 431.
15. The expression cassette of any of the proceeding paragraphs, wherein the promoter is a liver-specific promoter.
16. The expression cassette of any of the proceeding paragraphs, wherein the liver specific promoter is selected from any of: SEQ ID NOS: 86, 88, 91-96, 146-150, 439-441, or 481-500, or a liver specific promoter having at least 80% sequence identity to SEQ ID NOs: 86, 88, 91-96, 146-150, 439-441, or 481-500.
17. The expression cassette of any of the proceeding paragraphs, wherein the liver specific promoter is selected from any of: SEQ ID NOS: 98 or 99, or a liver specific promoter having at least 80% sequence identity to SEQ ID NOs: 98 or 99.
18. The expression cassette of any of the proceeding paragraphs, wherein the liver specific promoter is SEQ ID NOS: 97, or a liver specific promoter having at least 80% sequence identity to SEQ ID NO: 97.
19. The expression cassette of any of the proceeding paragraphs, further comprising one or more additional regulatory elements and/or a poly A sequence.
20. The expression cassette of any of the proceeding paragraphs, wherein the one or more additional regulatory elements is selected from the group consisting of an enhancer, a 5′ untranslated region (5′UTR), an intron, a reverse RNA pol II terminator sequence, and combinations thereof.
21. A recombinant adeno-associated virus (rAAV) vector comprising in its genome the expression cassette of any of the proceeding paragraphs.
22. A recombinant adeno-associated virus (rAAV) vector comprising in its genome:
   a) 5′ and 3′ AAV inverted terminal repeats (ITR) sequences; and
   b) located between the 5′ and 3′ ITRs, the expression cassette specified in any of the proceeding paragraphs.
23. The rAAV vector of any of the proceeding paragraphs, wherein the AAV genome further comprises at least one of:
   a) a 5′ ITR;
   b) an 5′ UTR sequence;
   c) an intron;
   d) a poly A sequence;
   e) a reverse RNA pol II terminator sequence; and
   f) a 3′ ITR.
24. The rAAV vector of any of the proceeding paragraphs, wherein the AAV genome comprises, in the 5′ to 3′ direction:
   a) a 5′ ITR;
   b) a liver-specific promoter;
   c) a 5′ UTR sequence;
   d) an intron;
   e) a codon-optimized nucleic acid specified in any of the proceeding paragraphs;
   f) a poly A sequence;
   g) a reverse RNA pol II terminator sequence; and
   h) a 3′ ITR.
25. The rAAV vector of any of the proceeding paragraphs, wherein the 5′ UTR sequence comprises SEQ ID NO: 41, or a nucleic acid having at least 90% sequence identity to SEQ ID NO: 41.
26. The rAAV vector of any of the proceeding paragraphs, wherein the 5′ UTR sequence comprises SEQ ID NO: 40, or a nucleic acid having at least 90% sequence identity to SEQ ID NO: 40.
27. The rAAV vector of any of the proceeding paragraphs, wherein the intron is selected from the group consisting of a MVM sequence, a HBB2 sequence, an CMVIE intron sequence, a UBC intron sequence, and a SV40 sequence.
28. The rAAV vector of any of the proceeding paragraphs, wherein the 3′ UTR sequence is located 3′ of the codon-optimized nucleic acid and 5′ of the 3′ ITR sequence, or is located between the codon-optimized nucleic acid and the poly A sequence.
29. The rAAV vector of any of the proceeding paragraphs, wherein the heterologous, codon-optimized nucleic acid sequence further comprises a 3′ intron sequence, wherein the 3′ intron sequence is located 3′ of the nucleic acid encoding the FVIII polypeptide and 5′ of the 3′ ITR sequence, or is located between the nucleic acid encoding the FVIII polypeptide and the poly A sequence.
30. The rAAV vector of any of the proceeding paragraphs, wherein at least one of the 5′ ITR or 3′ITR comprises an insertion, deletion or substitution.
31. The rAAV vector of any of the proceeding paragraphs, wherein one or more CpG islands in the ITR are removed.
32. The rAAV vector of any of the proceeding paragraphs, wherein the poly A sequence is a full length HGF poly A sequence.
33. The rAAV vector of any of the proceeding paragraphs, wherein poly A sequence is selected from SEQ ID NO: 42-44 or 514, or a nucleic acid sequence at least 80% sequence identity to SEQ ID NOS: 42-44 or 514.
34. The rAAV vector of any of the proceeding paragraphs, wherein the reverse RNA pol II terminator sequence is SEQ ID NO: 45, or a nucleic acid sequence at least 80% sequence identity to SEQ ID NOS: 45.
35. The rAAV vector of any of the proceeding paragraphs, wherein the rAAV vector is a chimeric AAV vector, haploid AAV vector, a hybrid AAV vector or polyploid AAV vector.
36. The rAAV vector of any of the proceeding paragraphs, wherein the rAAV vector is a rational haploid vector, a mosaic AAV vector, a chemically modified AAV vector, or a AAV vector from any AAV serotypes.
37. The rAAV vector of any of the proceeding paragraphs, wherein the rAAV vector is selected from the group consisting of: a AAVXL32 vector, a AAVXL32.1 vector, a AAV8 vector, or a haploid AAV8 vector comprising at least one AAV8 capsid protein.
38. The rAAV vector of any of the proceeding paragraphs, that has a capsid comprising capsid proteins from a serotype shown in Table 3 or a chimera thereof.
39. The rAAV vector of any of the proceeding paragraphs, wherein the capsid proteins are serotype AAV3b.
40. The rAAV vector of any of the proceeding paragraphs, wherein the AAV3b serotype capsid protein comprises one or more mutations selected from any of: 265D, 549A, Q263Y
41. The rAAV vector of any of the proceeding paragraphs, wherein the AAV3b serotype is selected from any of: AAV3b265D, AAV3b265D549A, AAV3b549A or AAV3bQ263Y, or AAV3bSASTG.
42. A pharmaceutical composition comprising the rAAV vector of any of the proceeding paragraphs in a pharmaceutically acceptable carrier.
43. A method for treating a subject in need of FVIII, the method comprising administering the rAAV vectors of any of the proceeding paragraphs or the pharmaceutical composition of any of the proceeding paragraphs, or the expression cassette of any of the proceeding paragraphs or the codon-optimized nucleic acid of any of the proceeding paragraphs, to the subject.
44. A method for treating hemophilia A, the method comprising administering the rAAV vectors of any one any of the proceeding paragraphs or the pharmaceutical composition of any of the proceeding paragraphs, or the expression cassette of any of the proceeding paragraphs or the codon-optimized nucleic acid of any of the proceeding paragraphs, to the subject.
45. The method of any of the proceeding paragraphs, wherein the AAV vector is manufactured from the plasmid of SEQ ID NO: 27.
46. The method of any of the proceeding paragraphs, wherein the encoded FVIII polypeptide is secreted from the subject's liver.
47. The method of any of the proceeding paragraphs, wherein administering to the subject is by systemic administration.
48. The method of any of the proceeding paragraphs, wherein the systemic administration is by intravenous administration.
49. The method of any of the proceeding paragraphs, wherein administering to the subject is by local administration.
50. The method of any of the proceeding paragraphs, wherein the local administration is by injection to the liver.
51. The method of any of the proceeding paragraphs, where the rAAV vector is administered at a dosage range of between 1.0E9 vg/kg to 5.0E12 vg/kg.
52. Use of a rAAV vector in the preparation of a medicament for treating subject in need of FVIII, the medicament comprising the rAAV vector specified in of any of the proceeding paragraphs.
53. Use of a rAAV vector in the preparation of a medicament for treating hemophilia A, the medicament comprising the rAAV vector specified in of any of the proceeding paragraphs.
54. An expression cassette containing the codon-optimized nucleic acid of any of the proceeding paragraphs, operably linked to a liver-specific promoter, wherein the liver specific promoter is selected from any of: SEQ ID NOS: 481-500, or a liver specific promoter having at least 80% sequence identity to SEQ ID NOs: 481-500.
55. An expression cassette containing the codon-optimized nucleic acid of any of the proceeding paragraphs, operably linked to a liver-specific promoter, wherein the liver specific promoter is selected from any of: SEQ ID NOS: 481-483, or a liver specific promoter having at least 80% sequence identity to SEQ ID NOs: 481-483.
56. A recombinant adeno-associated virus (rAAV) vector comprising in its genome the expression cassette of any of the proceeding paragraphs.

### EXAMPLES

### Example 1

### Introduction

In an effort to improve in vivo expression of therapeutic human Factor III (FVIII) delivered by an AAV vector, codon optimization of a nucleic acid encoding a functional variant of native human FVIII was pursued. The functional variant was a B-domain deleted APC resistant variant (FVIII-R355Q/R581Q) that has demonstrated approximately 5-fold increased procoagulant function relative to wild type B-domain deleted FVIII in injury models in hemophilia A (HA) mice (Wilhelm et al., 2021). This polypeptide is referred to as BDD-FVIII-QQ, or FVIII-QQ for simplicity. The amino acid sequence of the polypeptide is set forth in SEQ ID NO: 19. A series of nucleic acids encoding FVIII-QQ was generated from the codon-optimization process. The codon optimization process involved removal of all CpG's, minimization of alternative open reading frames, maximization of sequence diversity. 18 coding sequences encoding FVIII-QQ (also referred to herein as the human FVIII polypeptide) and assessed for potency with in vitro and in vivo assays. These nucleic acids were then tested for expression of the encoded protein, for their potential use in the setting of AAV-mediated gene therapy.

### Materials and Methods

Test articles. The nucleic acid sequence that encodes a B domain deleted variant of FVIII was adapted by modifying two codons (355 and 581) to encode Q rather than R (R355Q/R581Q) so that the nucleic acid sequence now encoded the B domain deleted FVIII-QQ variant. This nucleic acid sequence (referred to as FVIII-QQ00) served as a benchmark for the codon optimization process. Eighteen coding sequences encoding FVIII-QQ were generated using a proprietary pipeline. These sequences are referred to consecutively from FVIII-QQ01 to QQ18. The nucleotide sequences are shown in Table 3 and correspond to sequences set forth in SEQ ID NOS: 1-18, respectively. Nucleic acids with the respective sequences and the baseline sequence were synthesized by GeneWiz and placed into an AAV based expression plasmid under the control of a liver-specific promoter (McIntosh, 2013), thereby generating a total of 19 different plasmids. These plasmids were identical except for the inserted FVIII-QQ encoding nucleic acids.

Plasmids. AAV based plasmids were generated from the 18 new sequences and the benchmark sequence. The nucleic acid sequences were operatively linked to a liver specific promoter (McIntosh et al.,), SEQ ID NO: 106, and a polyA signal (Levitt et al.). SEQ ID NO: 21 provides the plasmid sequence containing an exemplary codon-optimized sequence.

AAV production. The plasmids used in the in vitro and the in vivo hydrodynamic tail vein injection experiments were used to manufacture rAAV2/8 AAV vector particles. rAAV vector particles were produced by triple transfection in high density Pro10 cells, helper plasmid xx680 and repcap plasmid GSK2/8 were used. The cells were harvested on day three and lysed by sonication. The lysates were purified by iodixanol gradient and concentrated on amicon filtration units. The produced AAVs were quantified by sybr green ITR qPCR and their purity was confirmed by silver stain.

In vitro transfection. For assessment of expression of the FVIII-QQ from each of the different plasmid in cell culture, HepG2 and Huh7 cells were transfected with each of the 19 (18 novel+1 benchmark) different plamids. Two days after transfection, FVIII activity was analyzed using a chromogenic assay kit for the in vitro diagnostic photometric determination of FVIII activity in citrated plasma (Chromogenix Coatest® SP4 Factor VIII, Diapharma®), following the manufacturer's instructions. The cells were also co-transfected with a plasmid encoding secreted nanoluciferase. Luciferase expression levels was used both as a positive control for transfection and to normalize FVIII expression across the different plasmids. Studies were run in duplicate for each cell type.

Huh7 cells were cultured in DMEM medium supplemented with 10% Fetal Bovine Serum, 1% Penicillin/streptomycin (complete DMEM). On Day 1, 6×10⁵ Huh7 cells/well were seeded in 2 ml of complete DMEM in 6-well plates. Similarly, HepG2 cells were cultured in MEM medium supplemented with 10% Fetal Bovine Serum, 1% Penicillin/streptomycin, 1× non-essential amino acid and 1×GlutaMAX. On Day 1, 1×10⁶ HepG2 cells/well were seeded in 2 ml of complete MEM in 6-well plates. On Day 2, cells were transfected with different plasmids encoding FVIII and a plasmid encoding secreted nLuc using Lipofectamine™ 3000 Transfection Reagent (L3000001, ThermoFisher). For FVIII, 2250 ng of plasmid per well were used. For nanoluc, 250 ng of plasmid per well were used. Finally, on Day 4 (2 days after DNA transfection), supernatants were collected. FVIII activity was assessed as described in the section below.

hFVIII activity measurement. FVIII activity was determined using a COATEST SP4 FVIII following the manufacturer's instructions. In brief, a standard curve was generated using calibrated human plasma. Reconstituted normal plasma was used to generate the highest point in the curve and serial dilutions were used to generate the rest of the standards. FVIII-deficient plasma with undetectable FVIII activity was used as negative control and normal plasma was used as positive control (104% of normal FVIII activity). Samples, standards and quality controls were diluted 1/80 in working buffer and maintained on ice. For FVIII activity determination, 50 μl of phospholipids:FIXa+FX mix were added to each well of a previously tempered (37° C.) 96-well plate. Then, 25 μl of sample, standard or controls were added, mixed by pipetting, and incubated 5 minutes at 37° C. Next, 25 μl of CaCl₂ (provided in the kit) were added to each well, mixed by pipetting and incubated exactly 10 minutes at 37° C. Afterwards, 50 μl of S-2765+I-2581 (provided in the kit) were added per well, mixed and incubated exactly 10 minutes at 37° C. Finally, 25 μl of acetic acid 20% (v/v) were added and mixed. Finally, plates were read at A405 nm and A490 nm within 4 hours after stopping the reaction. For the analysis, the A490 to A405 (A405 nm-A490 nm) values were subtracted. Two different standard curves were generated covering two ranges of FVIII activity, from 108% to 20% and from 20% to 1.2% of normal, as per the manufacturer's instructions. Samples were interpolated using the appropriate curve.

Hydrodynamic tail vein injection of Plasmid DNA (HTVi administration). 8-week-old B6N-Tyrc-Brd/BrdCrCrl male mice (N=5 per group) were used in hydrodynamic injection experiments. Before plasmid administration, animals were weighted to determine the exact volume for the injection. The volume administered was proportional to the weight of the animal, adjusting it to 100 ml/kg. E.g., for a 20 g mouse the injected volume was 2 ml. The dose of plasmid administered was 2 mg/kg, adjusting it to a final concentration of 20 μg/ml of the solution. For maximal transduction efficacy, the reagent was injected as rapidly as possible (5-7 seconds).

AAV administration. rAAV vectors were administered to 8- to 12-week-old male C57BL/6J mice (n=5 mice/vector) in a volume of 200 μL of phosphate-buffered saline/0.001% Pluronic via tail vein injection. The mice were administered 1×10¹¹ vg/animal of either one of the different AAV8 FVIII-QQ vectors via tail vein injection. Circulating FVIII antigen levels were measured two- and four-weeks following vector administration.

Sample collection. Blood samples were obtained from the AAV injected mice by venesection of the lateral vein at 2 weeks post AAV injection. At 4 weeks post injections blood was collected via terminal cardiac bleeding. This was performed under anesthesia using isoflurane. For HTVi, blood was collected retro-orbitally 24 hours post plasmid administration. 3.8% sodium citrate was added to sterile, low protein binding 1.5 mL tubes to a 1/10 final volume. Blood was then added, gently mixed, and kept at 4° C. until processing. Samples were immediately centrifuged at 10,000 G for 10 minutes at 4° C. and thereafter plasma was collected and stored at −80° C. for further analysis. For tissue collection, the liver was cut into 2 small pieces of −10 mg and the remaining liver collected in a separate tube. Samples were snap frozen in liquid nitrogen and stored at −80° C. for further analysis.

Human coagulation factor VIII ELISA. Quantification of hFVIII was based on an ELISA assay using the commercial kit Anti-hFVIII (F8C-EIA, Affinity Biologicals). The ELISA plate (442404, Thermo Fisher Scientific) was coated with the capture antibody diluted 1/100 in carbonate buffer and incubated at room temperature for 2 hours. Blocking was not required under the conditions described. The capture antibody was removed by washing three times with wash buffer (PBS tween; 0.1% v/v). Standards and samples were diluted in green sample diluent supplied by the manufacturer and placed in the appropriate wells. The plate was incubated at room temperature for 2 hours. After incubation, plates were washed 3 times with wash buffer and the pre-diluted detection antibody was added to each well. Plates were incubated for 60 minutes at room temperature. After the incubation time, plates were washed 3 times with wash buffer and TMB (A:51-2606KC; B: 51-2607KC; BD) was added to develop the plates. Following incubation for 20 minutes at room temperature, the reaction was stopped with H₂SO₄ 1M. Absorbance was measured at a wavelength of 450 nm in a microplate reader.

The hFVIII concentration of the samples was determined by interpolating their optical density (OD) on the curve generated by a simple nonlinear regression analysis (sigmoidal, 4PL, X is concentration) relating the OD of the standard dilutions to their concentration.

DNA extraction. The Maxwell® RSC Tissue DNA Kit was used for DNA extraction, following manufacturer's instructions. Briefly, a piece of liver (20 mg approximately) was put in a 1.5 mL tube, 80 μL of TE buffer were added and the sample was disrupted and homogenized using a pestle. The sample was added to the 1^{st} position of the cartridge and a plunger was placed on well 8. An empty elution tube was placed into the elution tube position and 100 μl of Elution Buffer were added to the bottom of each elution tube. The tissue DNA method was run and once the extraction process was finished, the elution tube containing DNA was stored at −20° C. until the presence of plasmid DNA in the sample was analyzed.

Quantification of plasmid DNA in tissues. Presence of plasmid DNA in liver was assessed by real-time quantitative polymerase chain reaction (qPCR). The primers and probes used were specific to the HLP promoter, common to all the plasmids. Primers and probes are shown below in Table 4.

TABLE 4
Specific probe and primers for HLP

| ID | Sequence |
|---|---|
| Probe_HLP | GGGGGCGACTCAGATCCCAGCC- |
| | FAM (SEQ ID NO: 107) |
| Forward_HLP | AGGACGCTGTGGTTTCTGAG |
| | (SEQ ID NO: 108) |
| Reverse_HLP | AGGTCACCCCAGTTATCGGA |
| | (SEQ ID NO: 109) |

Statistical analysis. Analysis was performed using GraphPad Prism version 9.3.1. Data are presented as mean±SD unless otherwise indicated.

### Results

Eighteen modified coding sequences encoding FVIII-QQ were generated using a proprietary pipeline. These sequences, referred to consecutively as FVIII-QQ01 to QQ18, were each inserted into an AAV based expression plasmid under the control of a liver specific promoter. The respective plasmids were then used to analyze the sequences for expression in in vitro and in vivo systems.

### In Vitro Analysis of Expression of FVIII-QQ

HepG2 and Huh7 cells were transfected with AAV based expression plasmids containing the different modified FVIII nucleic acids. The supernatant of the cells was then assessed for FVIII expression levels. The results are shown in FIG. 1 as fold-change compared with results from the reference plasmid FVIII-QQ00.

### In Vivo Analysis of Expression of FVIII-QQ—Hydrodynamic Tail Vein Injection of Plasmids

Mice (N=5 per group) were administered the different FVIII-QQ plasmids or an empty plasmid via hydrodynamic tail vein injection. Twenty-four hours after administration, citrate plasma samples were collected to analyze for the presence of circulating hFVIII as measured by ELISA. Preliminary analysis of the results indicated unexpected variability in detected levels within each plasmid group. This prompted us to further examine the mice for appropriate delivery of the plasmid.

Most of the hFVIII expressing plasmids resulted in plasma hVIII levels above background, although QQ16 and QQ18 resulted in minimally detectable levels.

The level of gene expression has been shown to decrease significantly with increasing injection time (Liu, Song, & Liu, 1999). Thus, it was hypothesized that the intra-group variability observed for the circulating hFVIII levels could be due to some animals having received a suboptimal hydrodynamic tail vein injection. To ensure the mice had been successfully dosed, DNA was isolated from livers following terminal sacrifice, and the amount of hFVIII plasmid in the livers was quantified by qPCR. The results showed that some of the mice had levels of plasmid DNA in the liver that suggested mis-injection. These mice had levels similar to that of the vehicle-injected animals and orders of magnitude lower than anticipated. The mice with a plasmid copy number <10000/liver sample were considered as having been mis-injected and were excluded from subsequent hFVIII analysis.

Results are presented in FIG. 2. The animals injected with the empty plasmid (vehicle) showed no evidence of human FVIII in the circulation. Most of the mice that received the plasmids exhibited hFVIII levels above background, although when comparing circulating hFVIII levels to the benchmark QQ00 plasmid group, no statistically significant differences were observed for groups QQ01 through QQ15 (FIG. 2). Of note, cassettes QQ16, QQ17, and QQ18 elicited extremely low levels of hFVIII which cannot be attributed to failed plasmid injection. For all other animals that received a successful administration of the plasmid, circulating hFVIII levels above background were observed.

In summary, except for mice in groups QQ16, QQ17, and QQ18, all animals that were successfully administered FVIII-encoding plasmids via hydrodynamic tail vein injection had circulating levels of human FVIII above background levels.

### In Vivo Analysis of Expression of FVIII-QQ—Injection of AAV Particles

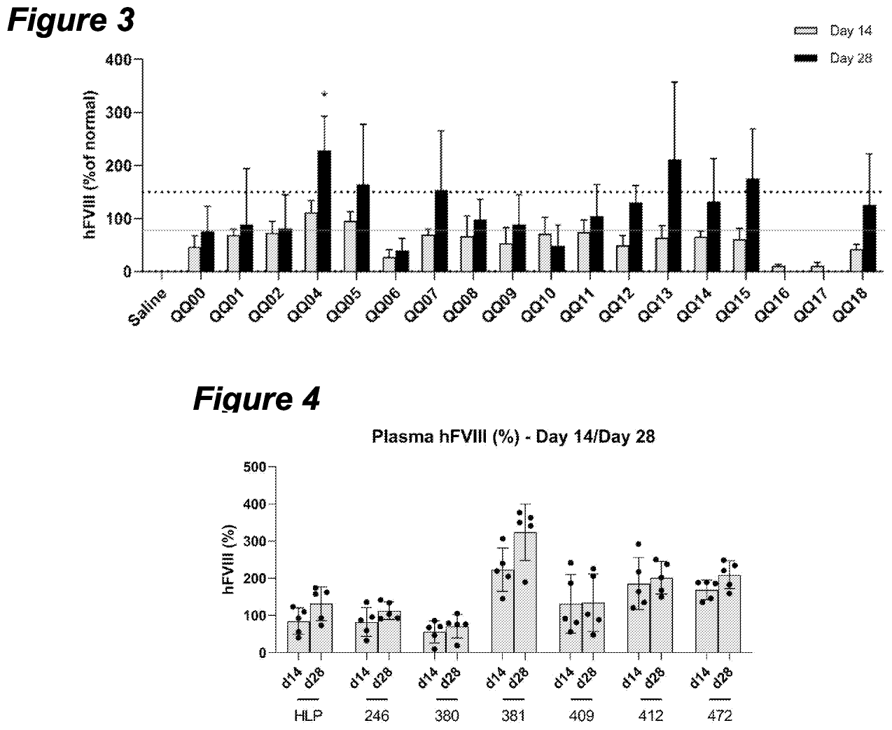

rAAV8 vector particles were generated using all 18 FVIII-QQ sequences and the FVIII-QQ00 benchmark. C57/B16 mice were administered the different AAV8 FVIII-QQ vectors via tail vein injection. Circulating FVIII antigen levels were measured two- (day 14) and four-weeks (day 28) following vector administration. Results are shown in FIG. 3. As expected, the vehicle-administered animals did not show measurable levels of human FVIII polypeptide in the circulation. Consistent with the in vitro plasmid transfection and the hydrodynamic injection studies shown above, vectors encoding the QQ16 and QQ17 elicited poor FVIII expression, i.e., below the limit of quantification at week 4. All other vectors, including the cassette encoding FVIII-QQ18 which had demonstrated subpar expression in the plasmid DNA experiments described above, elicited circulating human FVIII levels clearly above the baseline, although QQ6 and QQ10 expression was less than the benchmark QQ00.

At 4 weeks, expression levels of QQ1, QQ2, QQ4, QQ5, QQ7, QQ8, QQ9, QQ11, QQ12, QQ13, QQ14, QQ15, and QQ18 met or exceeded the expression levels of the benchmark QQ00, indicating the successful optimization of these nucleic acids. Further experimentation in other systems and/or with other regulatory elements may indicate even higher optimization than established here, especially for QQ1, QQ2, QQ8, QQ9, and QQ11. Alternatively, QQ4, QQ5, QQ7, QQ12, QQ13, QQ14, QQ15 and QQ18 exhibited fairly strong expression levels, as determined by the observation that they approached or exceeded a level that was 2-fold that of the benchmark QQ00 at 4 weeks. This indicates successful optimization of these nucleic acids to the point of superior expression. Similarly, QQ4, QQ5, QQ13 and QQ15 each exhibited more than 2-fold higher expression than the benchmark QQ00, which indicates successful optimization of these nucleic acids to the point of strongly superior expression. Only the FVIII-QQ04 showed circulating levels of hFVIII at week 4 statistically significantly above the FVIII-QQ00 group, with FVIII transgene levels of 228±64 and 76±47 percent of normal for FVIII-QQ04 and FVIII-QQ00, respectively. Other coding sequences such as QQ05, QQ13 and QQ15 also appeared to elicit FVIII levels numerically greater than those by the QQ00 benchmark but the difference did not reach statistical significance.

In summary, the preclinical studies presented here showed that the majority of the nucleic acids generated were capable of expressing active human FVIII.

### REFERENCES FOR EXAMPLE 1

Liu, F., Song, Y., & Liu, D. (1999). Hydrodynamics-based transfection in animals by systemic administration of plasmid DNA. Gene Ther, 6(7), 1258-1266. doi:10.1038/sj.gt.330094
Levitt et al., (1989). Genes Dev. 1989 July; 3(7):1019-25
McIntosh, J. (2013). Therapeutic levels of FVIII following a single peripheral vein administration of rAAV vector encoding a novel human factor VIII variant. Blood, 121, 3335-3344.
Wilhelm, A. R., Parsons, N. A., Samelson-Jones, B. J., Davidson, R. J., Esmon, C. T., Camire, R. M., & George, L. A. (2021). Activated protein C has a regulatory role in factor VIII function. Blood, 137(18), 2532-2543. doi:10.1182/blood.2020007562

### Example 2

Seven promoters and a state-of-the-art benchmark promoter (HLP) were placed upstream of a coding sequence encoding a FVIII sequence (e.g., F8-SQ00) and packaged within an AAV8 capsid; their potency was analyzed following intravenous injection of the AAV vectors to C57BL/6J mice over the course of 4 weeks. Table 5 summarizes the constructs described herein

TABLE 5
Constructs

| | | | Dose | |
|---|---|---|---|---|
| Vector ID | Promoter | CDS | (vg/kg) | Main readouts |
| Vector 1 | HLP | F8-SQ00 | 1 × 10¹⁰ | Blood collection at weeks 2 and 4 |
| Vector 1 Low dose | HLP | F8-SQ00 | 1.2 × 10⁹ | for measurement of human FVIII |
| Vector 5 | 246 | F8-SQ00 | 1 × 10¹⁰ | antigen levels by ELISA. |
| Vector 7 | 380 | F8-SQ00 | 1 × 10¹⁰ | Tissue collection at sacrifice for |
| Vector 8 | 381 | F8-SQ00 | 1 × 10¹⁰ | measurement of liver vector copy |
| Vector 12 | 409 | F8-SQ00 | 1 × 10¹⁰ | number. |
| Vector 14 | 412 | F8-SQ00 | 1 × 10¹⁰ | |
| Vector 17 | 472 | F8-SQ00 | 1 × 10¹⁰ | |
| Vector 24 | 412-UTR | F8-SQ00 | 1 × 10¹⁰ | |

### Methods

### AAV Production

AAV vectors described in this Example were produced by triple transfection in high density Pro10 cells. Helper plasmid xx680 and RepCap plasmid GSK2/8 were used. Cells were harvested on day three post-transfection and lysed via sonication. Lysates were purified by iodixanol gradient and concentrated via Amicon filtration units. The produced AAVs were quantified by PCR-based methods as described herein below and purity of each was confirmed by silver stain.

### AAV Titration

AAV vectors described herein in this Example were titrated by ITR droplet digital PCR (ddPCR). To extract the DNA from the AAV preparations, vectors were treated first with DNase and then with proteinase K. Next, the AAV DNA was quantified by ddPCR targeting the ITR region of the vector (Forward primer: 5′-GGAACCCCTAGTGATGGAGTT-3′ (SEQ ID NO: 466); Reverse primer: 5′-CGGCCTCAGTGAGCGA-3′ (SEQ ID NO: 467); Probe: 5′-FAM-CACTCCCTCTCTGCGCGCTCG-BHQ1-3′ (SEQ ID NO: 468)) and using an appropriate dilution.

### AAV Administration

AAV vectors described herein in this Example were administered to 8 to 12-week-old male C57BL/6JOlaHsd mice (n=5 mice/vector) in a volume of 200 uL of phosphate-buffered saline/0.001% Pluronic via tail vein injection. Dosage is indicated in Table 5.

### Sample Collection

Blood samples were obtained by venesection of the lateral vein at 2 weeks post AAV injection. At 4 weeks post injections, blood was collected via terminal cardiac bleeding performed under anesthesia using isoflurane. 3.8% sodium citrate was added to sterile, low protein binding 1.5 mL tubes to a 1/10 final volume. Blood was then added, gently mixed, and kept at 4° C. until processing. Samples were immediately centrifuged at 10,000 G for 10 minutes at 4° C.; plasma was collected and stored at −80° C. for further analysis. Tissue samples were harvested after sacrifice at week 4 from liver, heart, kidney, lung, brain, spleen, inguinal lymph nodes, adrenal glands, large intestine, testicles, quadriceps and gallbladder. For tissue collection, each tissue was divided in two, keeping one piece in a tube containing RNAlater® while the other was directly kept in an empty 1.5 mL tube. Both tubes were frozen in liquid nitrogen and stored at −80° C. for further analysis.

### Human Coagulation Factor VIII ELISA

Quantification of hFVIII levels was based on an ELISA assay using the commercial kit Anti-hFVIII (F8C-EIA, Affinity). The ELISA plate (442404, ThermoFisher) was coated with the capture antibody diluted 1/100 in carbonate buffer and incubated at room temperature for 2 hours. Blocking was not required under the conditions described. The capture antibody was removed by washing three times with wash buffer (PBS tween; 0.1% v/v). Standards and samples were diluted in green sample diluent supplied by the manufacturer and placed in the appropriate wells. The plate was incubated at room temperature for 2 hours. After incubation, plates were washed 3 times with wash buffer and the pre-diluted detection antibody was added to each well. Plates were incubated for 60 minutes at room temperature. After the incubation time, plates were washed 3 times with wash buffer and TMB (A:51-2606KC; B: 51-2607KC; BD) was added to develop the plates. Following incubation for 20 minutes at room temperature, the reaction was stopped with H2SO4 1M. Absorbance was measured at a wavelength of 450 nm in a microplate reader.

The hFVIII concentration of the samples was determined by interpolating their optical density (OD) on the curve generated by a simple nonlinear regression analysis (sigmoidal, 4PL, X is concentration) relating the OD of the standard dilutions to their concentration.

### DNA Extraction

Maxwell® RSC Tissue DNA Kit (Promega AS1610) was used for DNA extraction, following manufacturer's instructions. Briefly, a piece of liver (20 mg approximately) was put in a 1.5 mL tube, 80 mL of TE buffer were added and the sample was disrupted and homogenized using a pestle. The sample was added to the 1st position of the cartridge and a plunger was placed on well 8. An empty elution tube was placed into the elution tube position and 100 μl of Elution Buffer were added to the bottom of each elution tube. The tissue DNA method was run and once the extraction process was finished, the elution tube containing DNA was stored at −20° C. until the presence of vector DNA in the sample was analyzed.

### Hepatic Vector Copy Number Quantification (VCN)

VCN was determined by qPCR using the hF8co as target (GAPDH was used for normalization). The concentration of plasmid DNA was obtained by interpolating their Ct value on the standard curve generated by simple linear regression.

The primers and probes used for VCN determination are listed in Table 6.

TABLE 6
Specific probe and primers for hF8co sequence
used for VCN analysis

| ID | Sequence | |
|---|---|---|
| hF8co_Probe | CCCCTGGGCATGGAGAGCAA | SEQ ID NO: 469 |
| | GGCC-FAM | |
| hF8co_Forward | GATGGGCTGTGACCTGAACA | SEQ ID NO: 470 |
| hF8co_Reverse | CCAGGTGGCAAACATGTTGG | SEQ ID NO: 471 |
| Mouse GAPDH_ | 5HEX/TTGTCATCAACGGGA | SEQ ID NO: 472 |
| Probe | AGCCCATCA | |
| Mouse | ACGGCAAATTCAACGGCAC | SEQ ID NO: 473 |
| GAPDH_Forward | | |
| Mouse | TAGTGGGGTCTCGCTCCTGG | SEQ ID NO: 474 |
| GAPDH_Reverse | | |

### RNA Extraction

The Maxwell® RSC Tissue RNA Kit (#AS1340, Promega) was used for tissue RNA extraction, following manufacturer's instructions. Briefly, a piece of tissue was placed in a 1.5 mL tube, 200 mL of chilled 1-Thioglycerol/Homogenization solution were added and the sample was disrupted and homogenized using the TissueLyser II (Qiagen). 200 mL of Lysis buffer were added to the homogenate and vortexed for 15 seconds. The lysate was transferred to the cartridge, 10 mL of DNase I were added and the RNA was purified following the Maxwell® RSC simplyRNA method. The RNA was eluted in 50 μL of nuclease free water (NFW) and stored at −80° C. until the sample was quantified and analyzed.

In tissues with low RNA extraction yield, such as the muscle, TRIzol (#15596026, ThermoFisher) was used. Briefly, a piece of tissue was homogenized in 500 mL of TRIzol using the TissueLyser. After adding 500 mL of TRIzol, samples were incubated during 5 min at room temperature following a 10 min centrifugation at 4° C. and 12000 g. The supernatant was transferred to a new Eppendorf and incubated during 2 min at room temperature after adding 200 mL of chloroform. The non-coloured aqueous phase was mixed with 500 mL isopropanol, incubated during 10 min at room temperature and centrifuged for 10 min at 4° C. and 12000 g for RNA precipitation. Finally, the pellet was washed with 75% EtOH, dried at room temperature and resuspended in 50 mL NFW, following a final incubation of 2 min at 56° C.

### Reverse Transcription

1 μg RNA was reverse transcribed to its complementary DNA (cDNA) using a High Capacity cDNA Reverse Transcription Kit (#4368813, ThermoFisher) following the instructions indicated by the manufacturer.

### Gene Expression Determination

For gene expression determination, a quantitative polymerase chain reaction (qPCR) was performed using the TaqMan gene expression assay on the Applied Biosystems™ QuantStudio™ 5 system. The primers and probes used were specific to the hFVIII-SQ sequence, common to all the plasmids, and are listed in Table 7.

TABLE 7
Specific probe and primers

| ID | Sequence | |
|---|---|---|
| SQ_Probe | CCCCTGGGCATGGAGAGCAA | SEQ ID NO: 475 |
| | GGCC-FAM | |
| SQ_Forward | GATGGGCTGTGACCTGAACA | SEQ ID NO: 476 |
| SQ_Reverse | CCAGGTGGCAAACATGTTGG | SEQ ID NO: 477 |
| Rplp0_Probe | 5HEX/TGGAGGGTG/ZEN/ | SEQ ID NO: 478 |
| | TCCGCAACGTGGCC | |
| Rplp0_Forward | CAGGTGTTTGACAACGGCAG | SEQ ID NO: 479 |
| Rplp0_Reverse | CCGATCTGCAGACACACACT | SEQ ID NO: 480 |

The mRNA levels of the gene of interest were normalized with Rplp0 mRNA levels as endogenous control obtaining the relative expression by the ΔΔCt method (2-ΔΔCt) using the liver of each animal as reference sample to determine the fold decrease versus the liver. Samples were analyzed in triplicates and for each one no retrotranscribed RNA was used as negative control.

Statistical analysis was performed using GraphPad Prism version 9.3.1. Data are presented as mean±SD unless otherwise indicated.

### Results

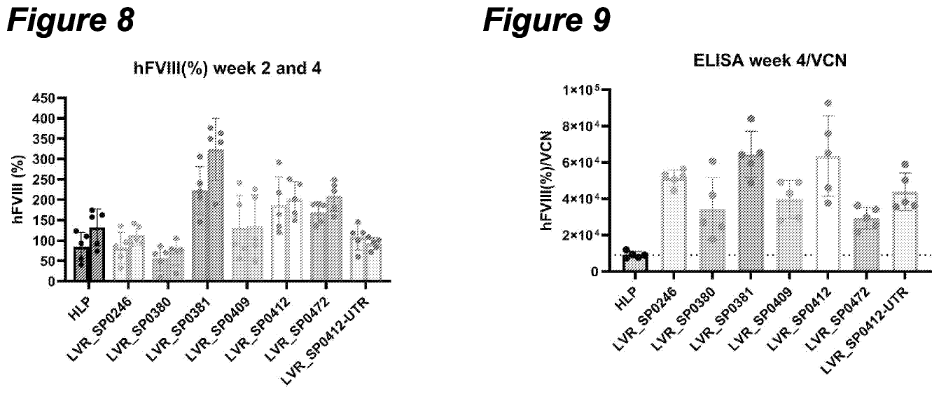

Seven liver-specific promoters were generated and placed upstream of a sequence encoding FVIII. The potency of these promoters at driving FVIII expression was benchmarked against HLP, a previously published promoter (McIntosh et al., 2013) and assessed following IV administration of AAV8 vectors. In summary, all vectors elicited circulating human FVIII levels above the baseline (see, e.g., FIGS. 4-9).

Two different doses of the vector containing the benchmark promoter (HLP) were used, resulting in the same FVIII/VCN ratio. These data show that VCN can effectively be used as a normalizer at the doses used. For an in vivo assessment of promoter potency, mice (N=5 per group) were intravenously administered either one of the different vectors. Two and four weeks post-administration, citrate plasma samples were collected to analyse for the presence of circulating hFVIII as measured by ELISA (FIG. 4). All vectors elicited circulating human FVIII levels above the baseline.

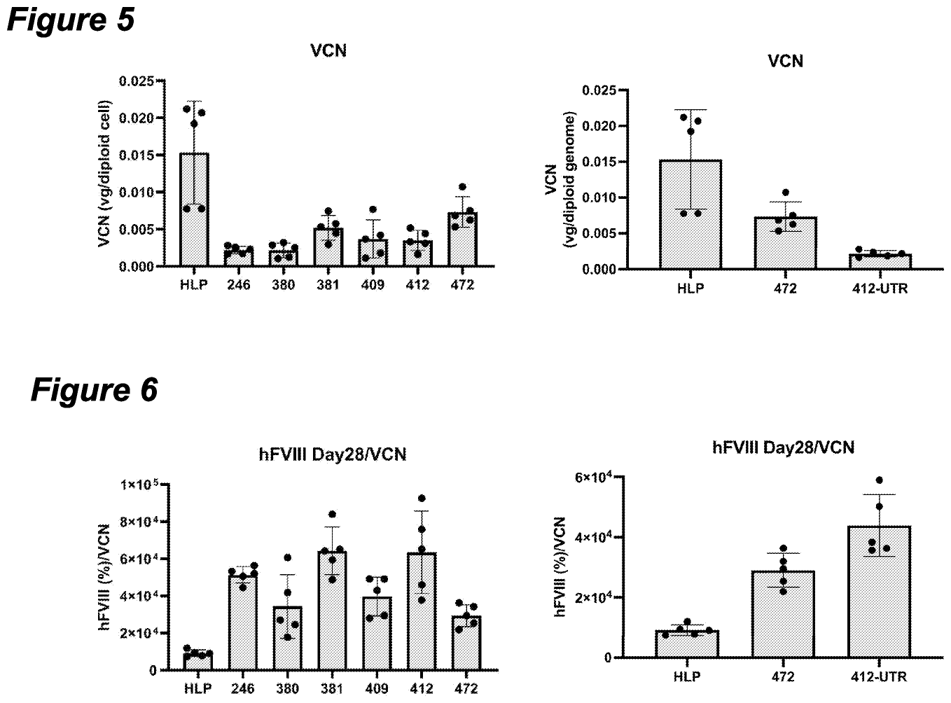

Following sacrifice at week 4 post-AAV administration, tissues were collected for further analysis. Firstly, vector copy number was assessed in the liver and is shown in FIG. 5.

To rule out that some of the apparent differences across promoters might be due to technical artifacts such as mis-titering of the viral preparation by qPCR, suboptimal dilutions of the test article or to slightly different injection volumes, we used the hepatic vector copy number to normalize the potency of each promoter (FIG. 6).

Of note, following normalization to vector copy number in each individual animal, both HLP groups showed the same expression levels regardless of vector dose, suggesting that at the doses used normalization to VCN is a useful tool to normalize expression to vector load. As shown in FIG. 6 above and summarized in Table 8 below, all the promoters tested showed increased potency vs the HLP benchmark.

TABLE 8
VCN-normalized hFVIII expression to the HLP group

| | FVIII Ag/VCN |
|---|---|
| | (relative to HLP) |
| HLP low dose | 1.1 |
| 246-hF8co | 5.6 |
| 380-hF8co | 3.8 |
| 381-hF8co | 7.0 |
| 409-hF8co | 4.3 |
| 412-hF8co | 6.9 |
| 472-hF8co | 3.2 |
| 412UTR-hF8co | 4.8 |

### Hepato-Specificity of the Promoters

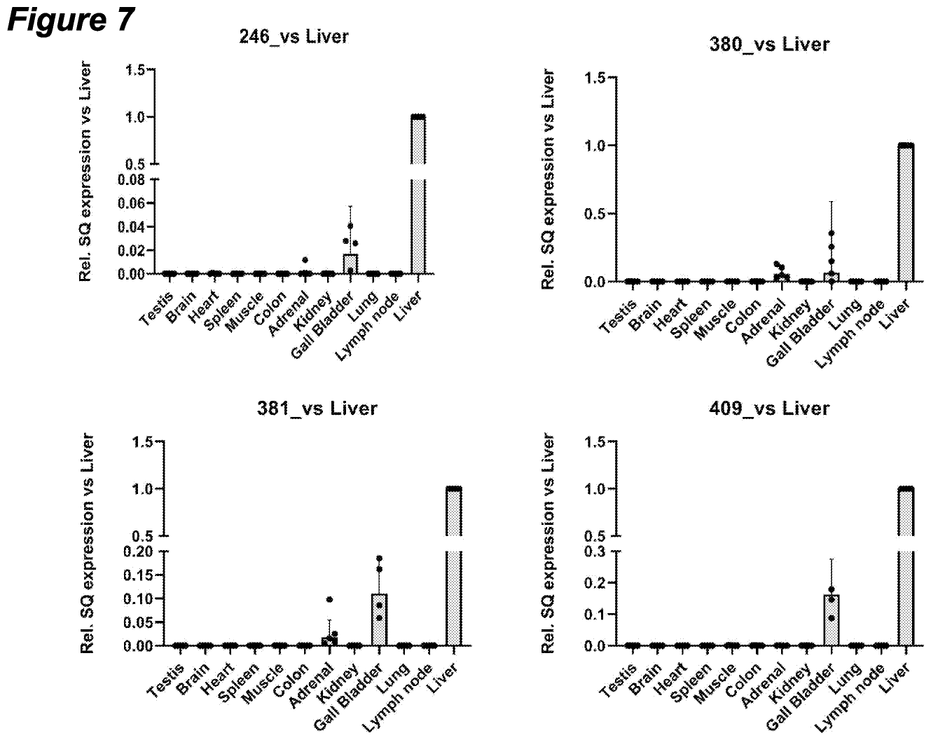

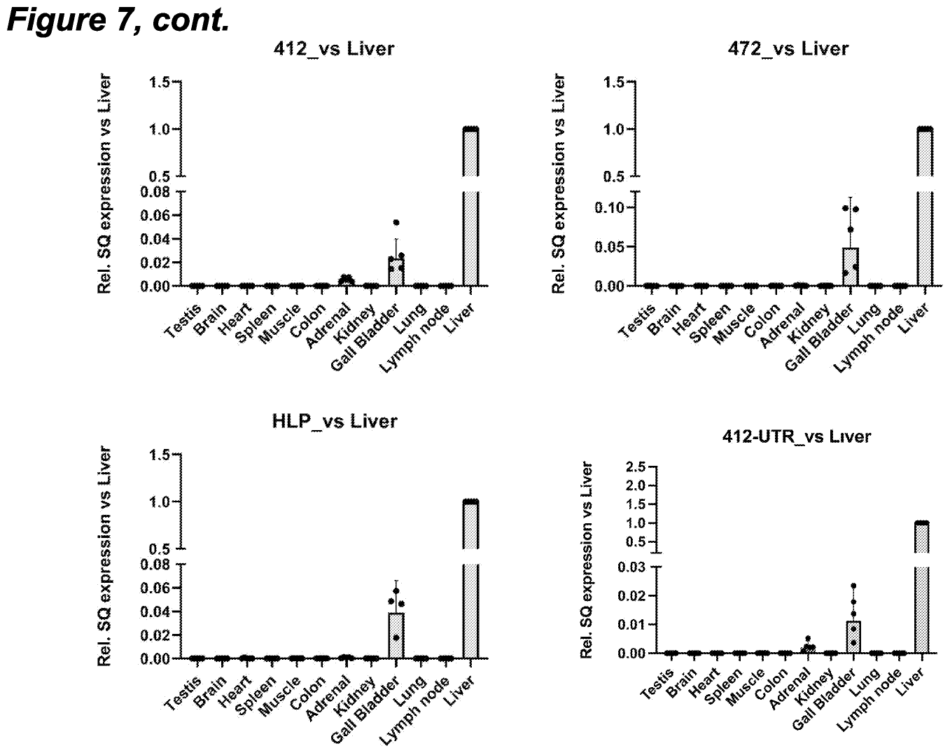

To assess the liver specificity of the promoters in this study, we evaluated F8 mRNA expression in liver and 11 non-hepatic tissues obtained at week 4 after sacrifice. PCR-based expression analysis was conducted to measure vector-derived FVIII mRNA relative to the expression of an endogenous reference gene. As expected, the liver was the organ with the highest levels of F8 mRNA followed by the gall bladder and the adrenal glands, with levels approximately 100-fold and 1000-fold lower depending on the promoter, respectively (FIG. 7). All other tissues had FVIII mRNA levels <10-5-fold compared to the liver. Importantly, vector-derived expression in brain and testis was over a million times lower vs liver. These data suggest that all the promoter assessed in the study are fundamentally hepato-specific.

In summary, the preclinical studies presented here showed that we have developed liver-specific promoters, some of which are substantially more potent at driving FVIII expression in mice that a benchmark promoter.

### REFERENCES FOR EXAMPLE 2

McIntosh, J., Lenting, P. J., Rosales, C., Lee, D., Rabbanian, S., Raj, D., . . . Nathwani, A. C. (2013). Therapeutic levels of FVIII following a single peripheral vein administration of rAAV vector encoding a novel human factor VIII variant. Blood, 121(17), 3335-3344.
Ward, N. J., Buckley, S. M. K., Waddington, S. N., Vabdendriessche T., Chuah, M. K. L., Nathwani, A. C., McIntosh, J., Tuddenham, E. G. D., Kinnon, C., Thrasher, A. J., McVey, J. H. (2011). Codon optimization of human factor VIII cDNAs leads to high-level expression. Blood, 117(3), 798-807.

### Example 3

Table 9 summarizes an additional study design described herein in Example 3. This example describes the generation of the various contructs that include a liver-specific promoter for driving expression of a codon-opitmized FVIII described herein for the assessment of their potency in an in vivo study in mice.

TABLE 9
Study design

| Promoter | CDS |
|---|---|
| TTR | F8-SQ |
| HLP | F8-SQ00 |
| 246 | F8-QQ04 |
| 246 | F8-QQ05 |
| 412 | F8-QQ04 |
| 412 | F8-QQ05 |
| 472 | F8-QQ04 |

### Methods

Methods for production of AAV expressing the constructs described in Table 9 and administration of the AAV were completed as described in Example 2.

AAV vectors described herein in this Example were administered to 8 to 12-week-old male C57BL/6JOlaHsd mice (n=5 mice/vector) in a volume of 200 μL of phosphate-buffered saline/0.001% Pluronic via tail vein injection. Dosage was 3e10 VG/mouse for all AAVs listed in Table 9. Two identical groups of mice were administered the contructs; one group was sacrificed at 4 weeks and the other was sacrificed at 8 weeks.

Sample collections were performed as described in Example 2. Additionally, all diagnostic assays performed, e.g., Human coagulation factor VIII ELISA, DNA extraction, Hepatic vector copy number quantification (VCN), RNA extraction, Reverse transcription, and Gene expression determination, were performed as described in Example 2. The primers and probes used for VCN determination are listed in Table 6. For gene expression determination, the primers and probes used were specific to the hFVIII-SQ sequence, common to all the plasmids, and are listed in Table 7.

Statistical analysis was performed using GraphPad Prism version 9.3.1. Data are presented as mean±SD unless otherwise indicated.

### Results

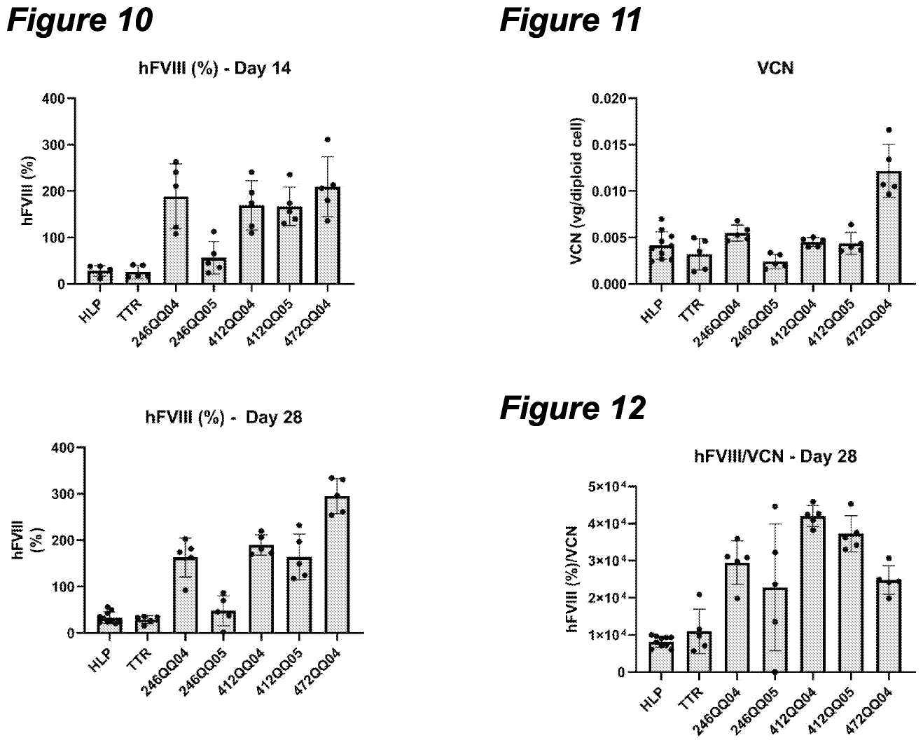

Liver-specific promoters SP0246 (SEQ ID NO: 481), SP0412 (SEQ ID NO: 482), and SP0472 (SEQ ID NO: 483) were placed upstream of a nucleic acid sequence encoding codon-opitmized FVIII F8-QQ04 or F8-QQ05. The potency of these promoters at driving codon-optimized FVIII expression was benchmarked against HLP as described above and assessed following IV administration of AAV8 vectors. In summary, all vectors elicited circulating human FVIII levels above the baseline (see, e.g., FIGS. 10-12).

For an in vivo assessment of promoter potency, mice (N=5 per group) were intravenously administered either one of the different vectors. Two and four weeks post-administration, citrate plasma samples were collected to analyse for the presence of circulating hFVIII as measured by ELISA (FIG. 10). FVIII levels in serum are expressed as a percentage of normal. The circulating levels of hFVIII (%) are calculated by interpolating the OD values to a standard curve (made using commercial FVIII) with experimental values, and using 1 IU/ml of the standard as equivalent to 100% of normal, see, e.g., McIntosh, J., et al. Gene therapy, Apr. 25, 2013, which is incorporated herein by reference in its entirety.

Following sacrifice at week 4 and 8 post-AAV administration, tissues were collected for further analysis. Firstly, vector copy number was assessed in the liver and is shown in FIG. 11. Similar results were found in each of the groups, i.e., the week 4 and 8 post-AAV administration.

To rule out that some of the apparent differences across promoters might be due to technical artifacts such as mis-titering of the viral preparation by qPCR, suboptimal dilutions of the test article or to slightly different injection volumes, we used the hepatic vector copy number to normalize the potency of each promoter (FIG. 12).

### Example 4

Table 10 summarizes an additional study design described herein in Example 4. This example describes the generation of the various contructs that include a liver-specific promoter for driving expression of a codon-opitmized FVIII described herein for the assessment of their potency in an in

TABLE 10
Study design

| Promoter | CDS |
|---|---|
| TTR | F8-SQ |
| 246 | F8-QQ04 |
| 412 | F8-QQ04 |
| 412 | F8-QQ05 |
| 472 | F8-QQ04 |

### Methods

Methods for production of AAV expressing the constructs described in Table 10 and administration of the AAV were completed as described in Example 2.

AAV vectors described herein in this Example were administered to 8 to 12-week-old male C57BL/6JOlaHsd mice (n=5 mice/vector) in a volume of 200 μL of phosphate-buffered saline/0.001% Pluronic via tail vein injection. Mice were injected with two different doses: one group was injected with 5e9 vg/mouse and 1.68e9 vg/mouse. Mice were sacrificed at 4 weeks.

AAV vectors described herein in this Example were titrated by ITR droplet digital PCR (ddPCR). To extract the DNA from the AAV preparations, vectors were treated first with DNase and then with proteinase K. Next, the AAV DNA was quantified by ddPCR targeting the ITR region of the vector (Forward primer: 5′-GGAACCCCTAGTGATGGAGTT-3′ (SEQ ID NO: 466); Reverse primer: 5′-CGGCCTCAGTGAGCGA-3′ (SEQ ID NO: 467); Probe: 5′-FAM-CACTCCCTCTCTGCGCGCTCG-BHQ1-3′ (SEQ ID NO: 468)) and using an appropriate dilution.

Sample collections were performed as described in Example 2. Additionally, all diagnostic assays performed, e.g., Human coagulation factor VIII ELISA, DNA extraction, Hepatic vector copy number quantification (VCN) were performed as described in Example 2. The primers and probes used for VCN determination are listed in Table 11 below.

Statistical analysis was performed using GraphPad Prism version 9.3.1. Data are presented as mean±SD unless otherwise indicated.

### Results

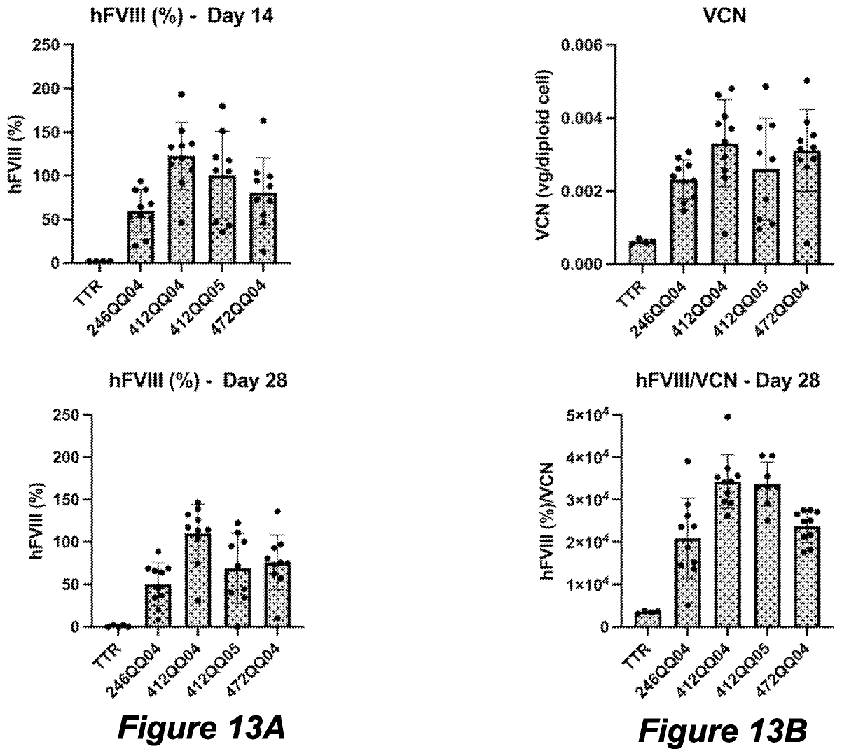

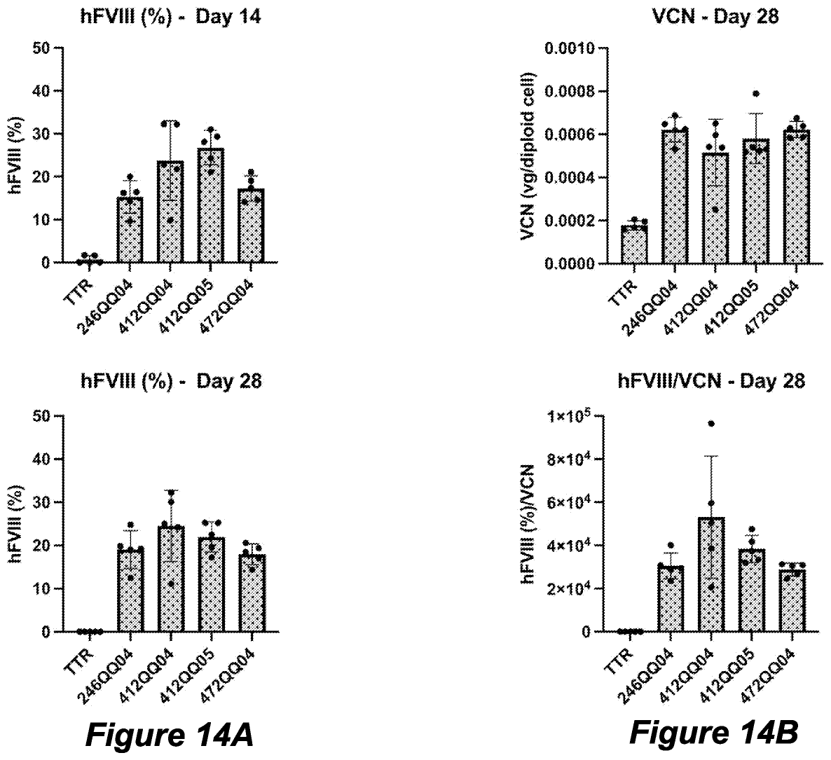

Liver-specific promoters SP0246 (SEQ ID NO: 481), SP0412 (SEQ ID NO: 482), and SP0472 (SEQ ID NO: 483) were placed upstream of a nucleic acid sequence encoding codon-optimized FVIII F8-QQ04 or F8-QQ05. The potency of these promoters at driving codon-optimized FVIII expression was benchmarked against TTR-SQ construct, a reference construct known in the prior art, as described above and assessed following IV administration of AAV8 vectors. In summary, all candidate vectors elicited circulating human FVIII levels above the baseline (see, e.g., FIGS. 13-14), and significantly more than the FVIII level expressed by the benchmark TTR-SQ construct.

Two different doses of the vectors were used, resulting in similar FVIII/VCN ratios. For an in vivo assessment of vector potency, mice (N=5 per group) were intravenously administered either one of the different vectors. Two and four weeks post-administration, citrate plasma samples were collected to analyse for the presence of circulating hFVIII as measured by ELISA (FIG. 13).

Following sacrifice at week 4 post-AAV administration, vector copy number was assessed in the liver and is shown in FIG. 13.

To rule out that some of the apparent differences across vectors might be due to technical artifacts such as mis-titering of the viral preparation by ddPCR, suboptimal dilutions of the test article or to slightly different injection volumes, we used the hepatic vector copy number to normalize the potency of each candidate (FIG. 14).

TABLE 11
Specific probe and primers for hF8co sequence
used for VCN analysis

| ID | Sequence | |
|---|---|---|
| hF8co_Probe | CCCCTGGGCATGGAGAGCAA | SEQ ID NO: 469 |
| | GGCC-FAM | |
| hF8co_Forward | GATGGGCTGTGACCTGAACA | SEQ ID NO: 470 |
| hF8co_Reverse | CCAGGTGGCAAACATGTTGG | SEQ ID NO: 471 |
| Mouse GAPDH_ | 5HEX/TTGTCATCAACGGGA | SEQ ID NO: 472 |
| Probe | AGCCCATCA | |
| Mouse | ACGGCAAATTCAACGGCAC | SEQ ID NO: 473 |
| GAPDH_Forward | | |
| Mouse | TAGTGGGGTCTCGCTCCTGG | SEQ ID NO: 474 |
| GAPDH_Reverse | | |
| hF8SQ_Probe | GTTTGAAACTGTGGAGATGC | SEQ ID NO: 501 |
| | TGCCCT-FAM | |
| hF8SQ_Forward | CCACTTCTCTGGGCATGTGT | SEQ ID NO: 502 |
| hF8SQ_Reverse | CCTCCAGATGCCAGCCTTAG | SEQ ID NO: 503 |

### Example 5

Nucleic acid sequences for novel, liver-specific promoters used in Example 2.

SP0246 (SEQ ID NO: 481)
(SEQ ID NO: 481)
AGGTTAATTTTTAAAAAGCAGTCAAAAGTCCAAGTGGCCCTTGGCAGCATTTACTCTCTCTGTTTGCT
CTGGTTAATAATCTCAGGAGCACAAACATTCCGGCCCGGGAGGCGCCCTTTGGACCTTTTGCAATCCT
GGCGCACTGAACCCTTGACCCCTGCCCTGCAGCCCCCGCAGCTTGCTGTTTGCCCACTCTATTTGCCC
AGCCCCAGGGGCATATAAAACAGGGGCAAGGCACAGACTCATAGCAGAGCAATCACCACCAAGCCTGG
AATAACTGCA
SP0412 (SEQ ID NO: 482)
(SEQ ID NO: 482)
AGGTTAATTTTTAAAAAGCAGTCAAAAGTCCAAGTGGCCCTTGGCAGCATTTACTCTCTCTGTTTGCT
CTGGTTAATAATCTCAGGAGCACAAACATTCCCCCTGTTCAAACATGTCCTAATACTCTGTCTCTGCA
AGGGTCATCAGTAGTTTTCCATCTTACTCAACATCCTCCCAGTGAGTCATATGTTTGCTCACTGAAGG
TTACTAGTTAACAGGCATCCCTTAAACAGGATATAAAAGGACTTCAGCAGGACTGCTCGAAACATCCC
ACTCA
SP0472 (SEQ ID NO: 483)
(SEQ ID NO: 483)
CCCTGTTCAAACATGTCCTAATACTCTGTCTCTGCAAGGGTCATCAGTAGTTTTCCATCTTACTCAAC
ATCCTCCCAGTGCGGGCGACTCAGATCCCAGCCAGTGGACTTAGCCCCTGTTTGCTCCTCCGATAACT
GGGGTGACCTTGGTTAATATTCACCAGCAGCCTCCCCCGTTGCCCCTCTGGATCCACTGCTTAAATAC
GGACGAGGACAGGGCCCTGTCTCCTCAGCTTCAGGCACCACCACTGACCTGGGACAGTGAATCG
SP0380 (SEQ ID NO: 484)
(SEQ ID NO: 484)
CCGCCCCCACTGAACCCTTGACCCCTGCCCTGCAGCCCCCGCAGCTTGCTGTTTGCCCACTCTATTTG
CCCAGCCCCAGCCCTGTTCAAACATGTCCTAATACTCTGTCTCTGCAAGGGTCATCAGTAGTTTTCCA
TCTTACTCAACATCCTCCCAGTGAGTCATATGTTTGCTCACTGAAGGTTACTAGTTAACAGGCATCCC
TTAAACAGGATATAAAAGGACTTCAGCAGGACTGCTCGAAACATCCCACT
SP0381 (SEQ ID NO: 485)
(SEQ ID NO: 485)
AAGCTTTCTGAACAGCCAAACAGAGATTCCAAAGTTCAGGCACCAAAGTTCAGACCCTAACAGTTATT
TACAAGGGTCAGTTAACCCCTGTTCAAACATGTCCTAATACTCTGTCTCTGCAAGGGTCATCAGTAGT
TTTCCATCTTACTCAACATCCTCCCAGTGAGTCATATGTTTGCTCACTGAAGGTTACTAGTTAACAGG
CATCCCTTAAACAGGATATAAAAGGACTTCAGCAGGACTGCTCGAAACATCCCACT
SP0409 (SEQ ID NO: 486)
(SEQ ID NO: 486)
AGGTTAATTTTTAAAAAGCAGTCAAAAGTCCAAGTGGCCCTTGGCAGCATTTACTCTCTCTGTTTGCT
CTGGTTAATAATCTCAGGAGCACAAACATTCCCCCTGTTCAAACATGTCCTAATACTCTGTCTCTGCA
AGGGTCATCAGTAGTTTTCCATCTTACTCAACATCCTCCCAGTGGGGCATATAAAACAGGGGCAAGGC
ACAGACTCATAGCAGAGCAATCACCACCAAGCCTGGAATAACTGCA
SP0411 (SEQ ID NO: 487)
(SEQ ID NO: 487)
CCCTGTTCAAACATGTCCTAATACTCTGTCTCTGCAAGGGTCATCAGTAGTTTTCCATCTTACTCAAC
ATCCTCCCAGTGAGTCATATGTTTGCTCACTGAAGGTTACTAGTTAACAGGCATCCCTTAAACAGGAT
ATAAAAGGACTTCAGCAGGACTGCTCGAAACATCCCACT

### Example 6

Additional nucleic acid sequences for novel, liver-specific promoters.

| | | SEQ ID | | CpG |
|---|---|---|---|---|
| NAME | SEQUENCE | NO: | Length | Content |
| SP0412_v2 | aggttaatttttaaaaagcagtcaaaagtccaagtggcccttggcagca | 488 | 318 | 2 |
| | tttactctctctgtttgctctggttaataatctcaggagcacaaacatt | | | |
| | ccccctgttcaaacatgtcctaatactctgtctctgcaagggtcatcag | | | |
| | tagttttccatcttactcaacatcctcccagtgagtcatatgtttgctc | | | |
| | actgaaggttactagttaacaggcatcccttaaacaggatataaaagga | | | |
| | cttcagcaggactgctcgaaacatcccactcaggtggattcttgggcat | | | |
| | ttgcttaagcttggtaccgccacc | | | |
| SP0382 | aggttaatttttaaaaagcagtcaaaagtccaagtggcccttggcagca | 489 | 229 | 4 |
| | tttactctctctgtttgctctggttaataatctcaggagcacaaacatt | | | |
| | ccggcccgggaggcgccctttggaccttttgcaatcctggcgtctagag | | | |
| | ggtatataatgggggccactagtgccagcagcagcctgaccacatctca | | | |
| | tcctcaatcaccaccaagcctggaataactgca | | | |
| SP0383 | aggttaatttttaaaaagcagtcaaaagtccaagtggcccttggcagca | 490 | 214 | 8 |
| | tttactctctctgtttgctctggttaataatctcaggagcacaaacatt | | | |
| | ccggcccgggaggcgccctttggaccttttgcaatcctggcgtctagag | | | |
| | ggtatataatgggggccagccgctggcgtttccgttgcttggatcagtc | | | |
| | taggtgcagctgccggat | | | |
| SP0471 | ccctgttcaaacatgtcctaatactctgtctctgcaagggtcatcagta | 491 | 226 | 6 |
| | gttttccatcttactcaacatcctcccagtgcgggcgactcagatccca | | | |
| | gccagtggacttagcccctgtttgctcctccgataactggggtgacctt | | | |
| | ggttaatattcaccagcagcctcccccgttgcccctctggatccactgc | | | |
| | ttaaatacggacgaggacagggccctgtct | | | |
| SP0472 | ccctgttcaaacatgtcctaatactctgtctctgcaagggtcatcagta | 483 | 268 | 7 |
| | gttttccatcttactcaacatcctcccagtgcgggcgactcagatccca | | | |
| | gccagtggacttagcccctgtttgctcctccgataactggggtgacctt | | | |
| | ggttaatattcaccagcagcctcccccgttgcccctctggatccactgc | | | |
| | ttaaatacggacgaggacagggccctgtctcctcagcttcaggcaccac | | | |
| | cactgacctgggacagtgaatcg | | | |
| SP0473 | aggttaatttttaaaaagcagtcaaaagtccaagtggcccttggcagca | 492 | 246 | 6 |
| | tttactctctctgtttgctctggttaataatctcaggagcacaaacatt | | | |
| | cccgggcgactcagatcccagccagtggacttagcccctgtttgctcct | | | |
| | ccgataactggggtgaccttggttaatattcaccagcagcctcccccgt | | | |
| | tgcccctctggatccactgcttaaatacggacgaggacagggccctgtc | | | |
| | t | | | |
| SP0474 | aggttaatttttaaaaagcagtcaaaagtccaagtggcccttggcagca | 493 | 288 | 7 |
| | tttactctctctgtttgctctggttaataatctcaggagcacaaacatt | | | |
| | cccgggcgactcagatcccagccagtggacttagcccctgtttgctcct | | | |
| | ccgataactggggtgaccttggttaatattcaccagcagcctcccccgt | | | |
| | tgcccctctggatccactgcttaaatacggacgaggacagggccctgtc | | | |
| | tcctcagcttcaggcaccaccactgacctgggacagtgaatcg | | | |
| SP0475 | aggttaatttttaaaaagcagtcaaaagtccaagtggcccttggcagca | 494 | 290 | 9 |
| | tttactctctctgtttgctctggttaataatctcaggagcacaaacatt | | | |
| | cctgtaccggcccgggaggcgccctttggaccttttgcaatcctggcgg | | | |
| | gcgactcagatcccagccagtggacttagcccctgtttgctcctccgat | | | |
| | aactggggtgaccttggttaatattcaccagcagcctcccccgttgccc | | | |
| | ctctggatccactgcttaaatacggacgaggacagggccctgtct | | | |
| SP0476 | cctgaagtccaaaggtagaggaaccgagtgtttaaaaattactgtggct | 495 | 247 | 2 |
| | gtggagtcaacatgatgtaaaaaaacaaacatttggataacaccaagaa | | | |
| | gccagatatggttgaaatgttgactggttgacaaaaataatttgggttg | | | |
| | cttaatggtgcacaaaggtaatgcgggcatataaaacaggggcaaggca | | | |
| | cagactcatagcagagcaatcaccaccaagcctggaataactgcagcca | | | |
| | cc | | | |
| SP0477 | ccgcccccactgaacccttgacccctgccctgcagcccccgcagcttgc | 496 | 326 | 4 |
| | tgtttgcccactctatttgcccagccccagcctgaagtccaaaggtaga | | | |
| | ggaaccgagtgtttaaaaattactgtggctgtggagtcaacatgatgta | | | |
| | aaaaaacaaacatttggataacaccaagaagccagatatggttgaaatg | | | |
| | ttgactggttgacaaaaataatttgggttgcttaatggtgcacaaaggt | | | |
| | aatgcgggcatataaaacaggggcaaggcacagactcatagcagagcaa | | | |
| | tcaccaccaagcctggaataactgcagccacc | | | |
| SP0478 | ccgcccccactgaacccttgacccctgccctgcagcccccgcagcttgc | 497 | 345 | 4 |
| | tgtttgcccactctatttgcccagccccagcctgaagtccaaaggtaga | | | |
| | ggaaccgagtgtttaaaaattactgtggctgtggagtcaacatgatgta | | | |
| | aaaaaacaaacatttggataacaccaagaagccagatatggttgaaatg | | | |
| | ttgactggttgacaaaaataatttgggttgcttaatggtgcacaaaggt | | | |
| | aatgcagtcatatgtttgctcactgaaggttactagttaacaggcatcc | | | |
| | cttaaacaggatataaaaggacttcagcaggactgctcgaaacatccca | | | |
| | ct | | | |
| SP0479 | tgctctctgacaaagatacggtgggtcccactgatgaactgtgctgcca | 498 | 316 | 8 |
| | cagtaaatgtagccactatgcctatctccattctgaagatgtgccgccc | | | |
| | ccactgaacccttgacccctgccctgcagcccccgcagcttgctgtttg | | | |
| | cccactctatttgcccagccccaggggcgactcagatcccagccagtgg | | | |
| | acttagcccctgtttgctcctccgataactggggtgaccttggttaata | | | |
| | ttcaccagcagcctcccccgttgcccctctggatccactgcttaaatac | | | |
| | ggacgaggacagggccctgtct | | | |
| SP0480 | tgctctctgacaaagatacggtgggtcccactgatgaactgtgctgcca | 499 | 247 | 3 |
| | cagtaaatgtagccactatgcctatctccattctgaagatgtgccgccc | | | |
| | ccactgaacccttgacccctgccctgcagcccccgcagcttgctgtttg | | | |
| | cccactctatttgcccagccccaggggcatataaaacaggggcaaggca | | | |
| | cagactcatagcagagcaatcaccaccaagcctggaataactgcagcca | | | |
| | cc | | | |
| SP0481 | tgctctctgacaaagatacggtgggtcccactgatgaactgtgctgcca | 500 | 266 | 4 |
| | cagtaaatgtagccactatgcctatctccattctgaagatgtgccgccc | | | |
| | ccactgaacccttgacccctgccctgcagcccccgcagcttgctgtttg | | | |
| | cccactctatttgcccagccccagagtcatatgtttgctcactgaaggt | | | |
| | tactagttaacaggcatcccttaaacaggatataaaaggacttcagcag | | | |
| | gactgctcgaaacatcccact | | | |

### Example 7—AAV Production

Derivation of suspension HEK293 cells from an adherent HEK293 Qualified Master Cell Bank. The derivation of the suspension cell line from the parental HEK293 Master Cell Bank (MCB), is performed in a Class 10,000 clean room facility. The derivation of the suspension cell line is carried out in a two phase process that involved first weaning the cells off of media containing bovine serum and then adapting the cells to serum free suspension media compatible with HEK293 cells. The suspension cell line is created as follows. First, a vial of qualified Master Cell Bank (MCB) is thawed and placed into culture in DMEM media containing 10% fetal bovine serum (FBS) and cultured for several days to allow the cells to recover from the freeze/thaw cycle. The MCB cells are cultured and passaged over a 4 week period while the amount of FBS in the tissue culture media is gradually reduced from 10% to 2.5%. The cells are then transferred from DMEM 2.5% FBS into serum free suspension media and grown in shaker flasks. The cells are then cultured in the serum-free media for another 3 weeks while their growth rate and viability is monitored. The adapted cells are then expanded and frozen down. A number of vials from this cell bank are subsequently thawed and used during process development studies to create a scalable manufacturing process using shaker flasks and wave bioreactor systems to generate rAAV vectors. Suspension HEK293 cells are grown in serum-free suspension media that supports both growth and high transfection efficiency in shaker flasks and wave bioreactor bags. Multitron Shaker Incubators (ATR) are used for maintenance of the cells and generation of rAAV vectors at specific rpm shaking speeds (based on cell culture volumes), 80% humidity, and 5% CO₂.

Transfection of suspension HEK293 cells. On the day of transfection, the cells are counted using a ViCell XR Viability Analyzer (Beckman Coulter) and diluted for transfection. To mix the transfection cocktail the following reagents are added to a conical tube in this order: plasmid DNA, OPTIMEM® I (Gibco) or OptiPro SFM (Gibco), or other serum free compatible transfection media, and then the transfection reagent at a specific ratio to plasmid DNA or, close ended linear duplexed DNA. The plasmid DNA or, close ended linear duplexed DNA of the transgene construct has a sequence comprising a heterologous nucleic acid sequence of any of the codon-optimized FVIII-QQ transgene as described herein (i.e., any of SEQ ID NOs 1, 2, 4, 5, 7-9, 11-15 or 18) operatively linked to any of the liver-specific promoters described herein (i.e., any of SEQ ID NOS: 86, 88, 91-96, 146-150, 439-441, or 481-500). The cocktail further comprises a Packaging plasmid encoding Rep2 and serotype-specific Cap2: AAV-Rep/Cap, and the Ad-Helper plasmid (XX680: encoding adenoviral helper sequences). The cocktail is inverted to mix prior to being incubated at room temperature. The transfection cocktail is then pipetted into the flasks and placed back in the shaker/incubator. All optimization studies are carried out at 30 mL culture volumes followed by validation at larger culture volumes. Cells are harvested 48 hours post-transfection.

Production of rAAV using wave bioreactor systems. Wave bags are seeded 2 days prior to transfection. Two days post-seeding the wave bag, cell culture counts are taken and the cell culture is then expanded/diluted before transfection. The wave bioreactor cell culture is then transfected. Cell culture is harvested from the wave bioreactor bag at least 48 hours post-induction.

Harvesting suspension cells from shaker flasks and wave bioreactor bags. 48 hours post-induction, cell cultures are collected into 500 mL polypropylene conical tubes (Corning) either by pouring from shaker flasks or pumping from wave bioreactor bags. The cell culture is then centrifuged at 655×g for 10 min using a Sorvall RC3C plus centrifuge and H6000A rotor. The supernatant is discarded, and the cells are resuspended in 1×PBS, transferred to a 50 mL conical tube, and centrifuged at 655×g for 10 min. At this point, the pellet could either be stored in NLT-60° C. or continued through purification.

Titering rAAV from cell lysate using qPCR. 10 mL of cell culture is removed and centrifuged at 655×g for 10 min using a Sorvall RC3C plus centrifuge and H6000A rotor. The supernatant is decanted from the cell pellet. The cell pellet is then resuspended in 5 mL of DNase buffer (5 mM CaCl₂, 5 mM MgCl₂, 50 mM Tris-HCl pH 8.0) followed by sonication to lyse the cells efficiently. 300 μl is then removed and placed into a 1.5 mL microfuge tube. 140 units of DNase I is then added to each sample and incubated at 37° C. for 1 hour. To determine the effectiveness of the DNase digestion, 4-5 ug of plasmid DNA is spiked into a non-transfected cell lysate with and without the addition of DNase. 50 μl of EDTA/Sarkosyl solution (6.3% sarkosyl, 62.5 mM EDTA pH 8.0) is then added to each tube and incubated at 70° C. for 20 minutes. 50 μl of Proteinase K (10 mg/mL) is then added and incubated at 55° C. for at least 2 hours. Samples are then boiled for 15 minutes to inactivate the Proteinase K. An aliquot is removed from each sample to be analyzed by qPCR. Two qPCR reactions are carried out in order to effectively determine how much rAAV vector is generated per cell.

Purification of rAAV from crude lysate. Each cell pellet is adjusted to a final volume of 10 mL. The pellets are vortexed briefly and sonicated for 4 minutes at 30% yield in one second on, one second off bursts. After sonication, 550 U of DNase is added and incubated at 37° C. for 45 minutes. The pellets are then centrifuged at 9400×g using the Sorvall RCSB centrifuge and HS-4 rotor to pellet the cell debris and the clarified lysate is transferred to a Type70Ti centrifuge tube (Beckman 361625). In regard to harvesting and lysing the suspension HEK293 cells for isolation of rAAV, one skilled in the art could use mechanical methods such as microfluidization or chemical methods such as detergents, etc., followed by a clarification step using depth filtration or Tangential Flow Filtration (TFF).

AAV vector purification. Clarified AAV lysate is purified by column chromatography methods as one skilled in the art would be aware of and described in the following manuscripts (Allay et al., Davidoff et al., Kaludov et al., Zolotukhin et al., Zolotukin et al, etc).

Titering rAAV using dot blot. 100 ul of DNase buffer (140 units DNase, 5 mM CaCl₂, 5 mM MgCl₂, 50 mM Tris-HCl pH 8.0) is added to each well of a 96-well microtiter plate. 1-3 μl or serial dilutions of virus is added to each well and incubated at 37° C. for 30 min. The samples are then supplemented with 15 ul Sarkosyl/EDTA solution (6.3% sarkosyl, 62.5 mM EDTA pH 8.0) and placed at 70° C. for 20 min. Next, 15 μl of Proteinase K (10 mg/mL) is added and incubated at 50° C. for at least 2 hours. 125 μl of NaOH buffer (80 mM NaOH, 4 mM EDTA pH 8.0) is added to each well. A series of transgene specific standards are created through a dilution series. NaOH buffer is then added and incubated. Nylon membrane is incubated at RT in 0.4 M Tris-HCl, pH 7.5 and then set up on dot blot apparatus. After a 10-15 minute incubation in NaOH buffer, the samples and standards are loaded into the dot blot apparatus onto the GeneScreen PlusR hybridization transfer membrane (PerkinElmer). The sample is then applied to the membrane using a vacuum. The nylon membrane is soaked in 0.4 M Tris-HCl, pH 7.5 and then cross linked using UV strata linker 1800 (Stratagene) at 600 ujouls×100. The membrane is then pre-hybridized in CHURCH buffer (1% BSA, 7% SDS, 1 mM EDTA, 0.5 M Na₃PO₄, pH 7.5). After pre-hybridization, the membrane is hybridized overnight with a ³²P-CTP labeled transgene probe (Roche Random Prime DNA labeling kit). The following day, the membrane is washed with low stringency SSC buffer (1×SSC, 0.1% SDS) and high stringency (0.1×SSC, 0.1% SDS). It is then exposed on a phosphorimager screen and analyzed for densitometry using a STORM840 scanner (GE).

Analyzing rAAV vector purity using silver stain method. Samples from purified vector are loaded onto NuPage 10% Bis-Tris gels (Invitrogen) and run using 1× NuPage running buffer. Typically, 1×10¹⁰ particles are loaded per well. The gels are treated with SilverXpress Silver staining kit #LC6100 (Invitrogen).

Transduction Assays. HeLaRC-32 cells (Chadeuf et al., J Gene Med. 2:260 (2000)) are plated at 2×10⁵ cells/well of a 24 well plate and incubated at 37° C. overnight. The cells are observed for 90-100% confluence. 50 mL of DMEM with 2% FBS, 1% Pen/Strep is pre-warmed, and adenovirus (d1309) is added at a MOI of 10. The d1309 containing media is aliquoted in 900 μl fractions and used to dilute the rAAV in a series of ten-fold dilutions. The rAAV is then plated at 400 μl and allowed to incubate for 48 hours at 37° C.

Concentration Assays. The starting vector stock is sampled and loaded onto a vivaspin column and centrifuged at 470×g (Sorvall H1000B) in 10 minute intervals. Once the desired volume/concentration had been achieved, both sides of the membrane are rinsed with the retentate, which is then harvested. Samples of the pre-concentrated and concentrated rAAV are taken to determine physical titers and transducing units.

Transmission electron microscopy (TEM) of negatively stained rAAV particles. Electron microscopy allows a direct visualization of the viral particles. Purified dialyzed rAAV vectors are placed on a 400-mesh glow-discharged carbon grid by inversion of the grid on a 20 μl drop of virus. The grid is then washed 2 times by inversion on a 20 μl drop of ddH₂O followed by inversion of the grid onto a 20 μl drop of 2% uranyl acetate for 30 seconds. The grids are blotted dry by gently touching Whatman paper to the edges of the grids. Each vector is visualized using a Zeiss EM 910 electron microscope.

### Example 8—HLP-F8SQ

The HLP-F8SQ reference construct used throughout this specification has a sequence of SEQ ID NO: 505.

(SEQ ID NO: 505)
TCTAGAGCTAGCATATGGATCCATCGATTTAGGGATAACAGGGTAATTATCAGCACACAATTGCCCATTATACGC
GCGTATAATGGACTATTGTGTGCTGATATCTGTACACTTAAGGGCTAGATCTTAGCTTACGTCACTAGAGGGTCC
ACGTTTAGTTTTTAAGATCCATTGATCTCCTAAACGCTGCAAGATTCGCAACCTGGTATACTTAGCCTAGGCGCT
AGGTCCTAGTGCAGCGGGACTTTTTTTCTAAAGTCGTTGAGAGGAGGAGTCGTCAGACCAGATAGCTTTGATGTC
CTGATCGGAAGGATCGTTGGCCCCCCTGCAGGCAGCTGTTAATTAACTGCGCGCTCGCTCGCTCACTGAGGCCGC
CCGGGCAAAGCCCGGGCGTCGGGCGACCTTTGGTCGCCCGGCCTCAGTGAGCGAGCGAGCGCGCAGAGAGGGAGT
GGCCAACTCCATCACTAGGGGTTCCTGTTTGCTGCTTGCAATGTTTGCCCATTTTAGGGTGGACACAGGACGCTG
TGGTTTCTGAGCCAGGGGGCGACTCAGATCCCAGCCAGTGGACTTAGCCCCTGTTTGCTCCTCCGATAACTGGGG
TGACCTTGGTTAATATTCACCAGCAGCCTCCCCCGTTGCCCCTCTGGATCCACTGCTTAAATACGGACGAGGACA
GGGCCCTGTCTCCTCAGCTTCAGGCACCACCACTGACCTGGGACAGTGAATCGCCACCATGCAGATTGAGCTGAG
CACCTGCTTCTTCCTGTGCCTGCTGAGGTTCTGCTTCTCTGCCACCAGGAGATACTACCTGGGGGCTGTGGAGCT
GAGCTGGGACTACATGCAGTCTGACCTGGGGGAGCTGCCTGTGGATGCCAGGTTCCCCCCCAGAGTGCCCAAGAG
CTTCCCCTTCAACACCTCTGTGGTGTACAAGAAGACCCTGTTTGTGGAGTTCACTGACCACCTGTTCAACATTGC
CAAGCCCAGGCCCCCCTGGATGGGCCTGCTGGGCCCCACCATCCAGGCTGAGGTGTATGACACTGTGGTGATCAC
CCTGAAGAACATGGCCAGCCACCCTGTGAGCCTGCATGCTGTGGGGGTGAGCTACTGGAAGGCCTCTGAGGGGGC
TGAGTATGATGACCAGACCAGCCAGAGGGAGAAGGAGGATGACAAGGTGTTCCCTGGGGGCAGCCACACCTATGT
GTGGCAGGTGCTGAAGGAGAATGGCCCCATGGCCTCTGACCCCCTGTGCCTGACCTACAGCTACCTGAGCCATGT
GGACCTGGTGAAGGACCTGAACTCTGGCCTGATTGGGGCCCTGCTGGTGTGCAGGGAGGGCAGCCTGGCCAAGGA
GAAGACCCAGACCCTGCACAAGTTCATCCTGCTGTTTGCTGTGTTTGATGAGGGCAAGAGCTGGCACTCTGAAAC
CAAGAACAGCCTGATGCAGGACAGGGATGCTGCCTCTGCCAGGGCCTGGCCCAAGATGCACACTGTGAATGGCTA
TGTGAACAGGAGCCTGCCTGGCCTGATTGGCTGCCACAGGAAGTCTGTGTACTGGCATGTGATTGGCATGGGCAC
CACCCCTGAGGTGCACAGCATCTTCCTGGAGGGCCACACCTTCCTGGTCAGGAACCACAGGCAGGCCAGCCTGGA
GATCAGCCCCATCACCTTCCTGACTGCCCAGACCCTGCTGATGGACCTGGGCCAGTTCCTGCTGTTCTGCCACAT
CAGCAGCCACCAGCATGATGGCATGGAGGCCTATGTGAAGGTGGACAGCTGCCCTGAGGAGCCCCAGCTGAGGAT
GAAGAACAATGAGGAGGCTGAGGACTATGATGATGACCTGACTGACTCTGAGATGGATGTGGTGAGGTTTGATGA
TGACAACAGCCCCAGCTTCATCCAGATCAGGTCTGTGGCCAAGAAGCACCCCAAGACCTGGGTGCACTACATTGC
TGCTGAGGAGGAGGACTGGGACTATGCCCCCCTGGTGCTGGCCCCTGATGACAGGAGCTACAAGAGCCAGTAACT
GAACAATGGCCCCCAGAGGATTGGCAGGAAGTACAAGAAGGTCAGGTTCATGGCCTACACTGATGAAACCTTCAA
GACCAGGGAGGCCATCCAGCATGAGTCTGGCATCCTGGGCCCCCTGCTGTATGGGGAGGTGGGGGACACCCTGCT
GATCATCTTCAAGAACCAGGCCAGCAGGCCCTACAACATCTACCCCCATGGCATCACTGATGTGAGGCCCCTGTA
CAGCAGGAGGCTGCCCAAGGGGGTGAAGCACCTGAAGGACTTCCCCATCCTGCCTGGGGAGATCTTCAAGTACAA
GTGGACTGTGACTGTGGAGGATGGCCCCACCAAGTCTGACCCCAGGTGCCTGACCAGATACTACAGCAGCTTTGT
GAACATGGAGAGGGACCTGGCCTCTGGCCTGATTGGCCCCCTGCTGATCTGCTACAAGGAGTCTGTGGACCAGAG
GGGCAACCAGATCATGTCTGACAAGAGGAATGTGATCCTGTTCTCTGTGTTTGATGAGAACAGGAGCTGGTACCT
GACTGAGAACATCCAGAGGTTCCTGCCCAACCCTGCTGGGGTGCAGCTGGAGGACCCTGAGTTCCAGGCCAGCAA
CATCATGCACAGCATCAATGGCTATGTGTTTGACAGCCTGCAGCTGTCTGTGTGCCTGCATGAGGTGGCCTACTG
GTACATCCTGAGCATTGGGGCCCAGACTGACTTCCTGTCTGTGTTCTTCTCTGGCTACACCTTCAAGCACAAGAT
GGTGTATGAGGACACCCTGACCCTGTTCCCCTTCTCTGGGGAGACTGTGTTCATGAGCATGGAGAACCCTGGCCT
GTGGATTCTGGGCTGCCACAACTCTGACTTCAGGAACAGGGGCATGACTGCCCTGCTGAAAGTCTCCAGCTGTGA
CAAGAACACTGGGGACTACTATGAGGACAGCTATGAGGACATCTCTGCCTACCTGCTGAGCAAGAACAATGCCAT
TGAGCCCAGGAGCTTCAGCCAGAACCCCCCAGTGCTGAAGAGGCACCAGAGGGAGATCACCAGGACCACCCTGCA
GTCTGACCAGGAGGAGATTGACTATGATGACACCATCTCTGTGGAGATGAAGAAGGAGGACTTTGACATCTACGA
CGAGGACGAGAACCAGAGCCCCAGGAGCTTCCAGAAGAAGACCAGGCACTACTTCATTGCTGCTGTGGAGAGGCT
GTGGGACTATGGCATGAGCAGCAGCCCCCATGTGCTGAGGAACAGGGCCCAGTCTGGCTCTGTGCCCCAGTTCAA
GAAGGTGGTGTTCCAGGAGTTCACTGATGGCAGCTTCACCCAGCCCCTGTACAGAGGGGAGCTGAATGAGCACCT
GGGCCTGCTGGGCCCCTACATCAGGGCTGAGGTGGAGGACAACATCATGGTGACCTTCAGGAACCAGGCCAGCAG
GCCCTACAGCTTCTACAGCAGCCTGATCAGCTATGAGGAGGACCAGAGGCAGGGGGCTGAGCCCAGGAAGAACTT
TGTGAAGCCCAATGAAACCAAGACCTACTTCTGGAAGGTGCAGCACCACATGGCCCCCACCAAGGATGAGTTTGA
CTGCAAGGCCTGGGCCTACTTCTCTGATGTGGACCTGGAGAAGGATGTGCACTCTGGCCTGATTGGCCCCCTGCT
GGTGTGCCACACCAACACCCTGAACCCTGCCCATGGCAGGCAGGTGACTGTGCAGGAGTTTGCCCTGTTCTTCAC
CATCTTTGATGAAACCAAGAGCTGGTACTTCACTGAGAACATGGAGAGGAACTGCAGGGCCCCCTGCAACATCCA
GATGGAGGACCCCACCTTCAAGGAGAACTACAGGTTCCATGCCATCAATGGCTACATCATGGACACCCTGCCTGG
CCTGGTGATGGCCCAGGACCAGAGGATCAGGTGGTACCTGCTGAGCATGGGCAGCAATGAGAACATCCACAGCAT
CCACTTCTCTGGCCATGTGTTCACTGTGAGGAAGAAGGAGGAGTACAAGATGGCCCTGTACAACCTGTACCCTGG
GGTGTTTGAGACTGTGGAGATGCTGCCCAGCAAGGCTGGCATCTGGAGGGTGGAGTGCCTGATTGGGGAGCACCT
GCATGCTGGCATGAGCACCCTGTTCCTGGTGTACAGCAACAAGTGCCAGACCCCCCTGGGCATGGCCTCTGGCCA
CATCAGGGACTTCCAGATCACTGCCTCTGGCCAGTATGGCCAGTGGGCCCCCAAGCTGGCCAGGCTGCACTACTC
TGGCAGCATCAATGCCTGGAGCACCAAGGAGCCCTTCAGCTGGATCAAGGTGGACCTGCTGGCCCCCATGATCAT
CCATGGCATCAAGACCCAGGGGGCCAGGCAGAAGTTCAGCAGCCTGTACATCAGCCAGTTCATCATCATGTACAG
CCTGGATGGCAAGAAGTGGCAGACCTACAGGGGCAACAGCACTGGCACCCTGATGGTGTTCTTTGGCAATGTGGA
CAGCTCTGGCATCAAGCACAACATCTTCAACCCCCCCATCATTGCCAGATACATCAGGCTGCACCCCACCCACTA
CAGCATCAGGAGCACCCTGAGGATGGAGCTGATGGGCTGTGACCTGAACAGCTGCAGCATGCCCCTGGGCATGGA
GAGCAAGGCCATCTCTGATGCCCAGATCACTGCCAGCAGCTACTTCACCAACATGTTTGCCACCTGGAGCCCCAG
CAAGGCCAGGCTGCACCTGCAGGGCAGGAGCAATGCCTGGAGGCCCCAGGTCAACAACCCCAAGGAGTGGCTGCA
GGTGGACTTCCAGAAGACCATGAAGGTGACTGGGGTGACCACCCAGGGGGTGAAGAGCCTGCTGACCAGCATGTA
TGTGAAGGAGTTCCTGATCAGCAGCAGCCAGAATGGCCACCAGTGGACCCTGTTCTTCCAGAATGGCAAGGTGAA
GGTGTTCCAGGGCAACCAGGACAGCTTCACCCCTGTGGTGAACAGCCTGGACCCCCCCCTGCTGACCAGATACCT
GAGGATTCACCCCCAGAGCTGGGTGCACCAGATTGCCCTGAGGATGGAGGTGCTGGGCTGTGAGGCCCAGGACCT
GTACTGAAATAAAAAGATCTTTATTTTCATTAGATCTGTGTGTTGGTTTTTTGTGTGAGGAACCCCTAGTGATGGA
GTTGGCCACTCCCTCTCTGCGCGCTCGCTCGCTCACTGAGGCCGGGCGACCAAAGGTCGCCCGACGCCCGGGCTT
TGCCCGGGCGGCCTCAGTGAGCGAGCGAGCGCGCAGTTAATTAAGGCGCCCTAGGCCGACCCTTAGACTCTGTAC
TCAGTTCTATAAACGAGCCATTGGATACGAGATCCGTAGATTGATAAGGGACACGGAATATCCCCGGACGCAATA
GACACCGGTGGACAGCTTGGTATCCTGAGCACAGTCGCGCGTCCGAATCTAGCTCTACTTTAGAGGCCCCGGATT
CTGATGGTCGTAGACCGCAGAACCGATTGGGGGGATGAGATCTACTAGTTATCAGCACACAATTGCCCATTATAC
GCGCGTATAATGGACTATTGTGTGCTGATATAGGGATAACAGGGTAATTCTAGAGCTAGCATATGGATCCATCGA

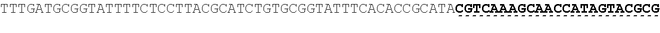

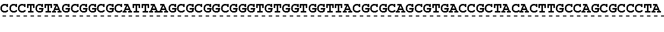

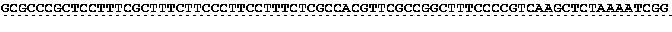

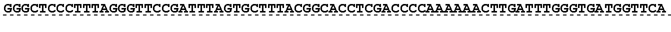

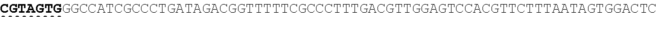

TTGTTCCAAACTGGAACAACACTCAACTCTATCTCGGGCTATTCTTTTGATTTATAAGGGATTTTGCCGATTTCG
GTCTATTGGTTAAAAAATGAGCTGATTTAACAAAAATTTAACGCGAATTTTAACAAAATATTAACGTTTACAATT
TTATGGTGCACTCTCAGTACAATCTGCTCTGATGCCGCATAGTTAAGCCAGCCCCGACACCCGCCAACACCCGCT
GACGCGCCCTGACGGGCTTGTCTGCTCCCGGCATCCGCTTACAGACAAGCTGTGACCGTCTCCGGGAGCTGCATG
TGTCAGAGGTTTTCACCGTCATCACCGAAACGCGCGAGACGAAAGGGCCTCGTGATACGCCTATTTTTATAGGTT
AATGTCATGATAATAATGGTTTCTTAGACGTCAGGTGGCACTTTTCGGGGAAATGTGCGCGGAACCCCTATTTGT
TTATTTTTCTAAATACATTCAAATATGTATCCGCTCATGAGACAATAACCCTGATAAATGCTTCAATAATATTGA

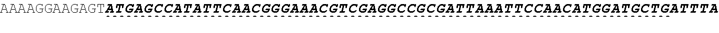

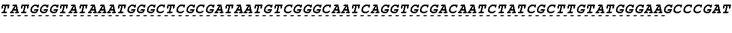

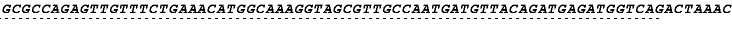

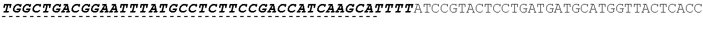

ACTGCGATCCCCGGAAAAACAGCATTCCAGGTATTAGAAGAATATCCTGATTCAGGTGAAAATATTGTTGATGCG
CTGGCAGTGTTCCTGCGCCGGTTGCATTCGATTCCTGTTTGTAATTGTCCTTTTAACAGCGATCGCGTATTTCGT
CTCGCTCAGGCGCAATCACGAATGAATAACGGTTTGGTTGATGCGAGTGATTTTGATGACGAGCGTAATGGCTGG
CCTGTTGAACAAGTCTGGAAAGAAATGCATAAACTTTTGCCATTCTCACCGGATTCAGTCGTCACTCATGGTGAT
TTCTCACTTGATAACCTTATTTTTGACGAGGGGAAATTAATAGGTTGTATTGATGTTGGACGAGTCGGAATCGCA
GACCGATACCAGGATCTTGCCATCCTATGGAACTGCCTCGGTGAGTTTTCTCCTTCATTACAGAAACGGCTTTTT
CAAAAATATGGTATTGATAATCCTGATATGAATAAATTGCAGTTTCATTTGATGCTCGATGAGTTTTTCTAAGCG
TATAATGGTCTAGAGCTAGCATATGGATCCATCGATTCCATTATACGCCTGTCAGACCAAGTTTACTCATATATA
CTTTAGATTGATTTAAAACTTCATTTTTAATTTAAAAGGATCTAGGTGAAGATCCTTTTTGATAATCTCATGACC

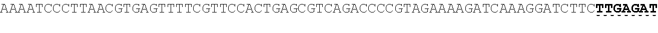

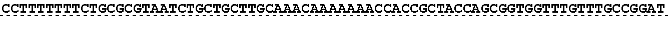

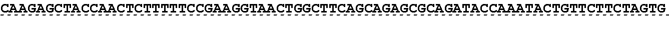

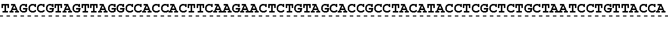

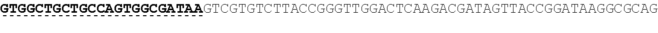

CGGTCGGGCTGAACGGGGGGTTCGTGCACACAGCCCAGCTTGGAGCGAACGACCTACACCGAACTGAGATACCTA
CAGCGTGAGCTATGAGAAAGCGCCACGCTTCCCGAAGGGAGAAAGGCGGACAGGTATCCGGTAAGCGGCAGGGTC
GGAACAGGAGAGCGCACGAGGGAGCTTCCAGGGGGAAACGCCTGGTATCTTTATAGTCCTGTCGGGTTTCGCCAC
CTCTGACTTGAGCGTCGATTTTTGTGATGCTCGTCAGGGGGGCGGAGCCTATGGAAAAACGCCAGCAACGCGGCC
TTTTTACGGTTCCTGGCCTTTTGCTGGCCTTTTGCTCACATGT

Table 12 describes various elements within the sequence of SEQ ID NO: 505.

In one embodiment, a construct described herein comprises the Kozak sequence, polyA sequence, left and right ITRs, origins of replication and kanamicin resistance sequences as described in Table 12.

In one embodiment, the only elements of within SEQ ID NO: 505 that are changed in the constructs described herein are the promoter element (e.g., HLP) and/or the open reading frame of the transgene (e.g., FVIII-SQ).

For example, a FVIII-QQ construct as described herein can be made by changing the the open reading frame of the transgene, e.g., FVIII-SQ, of SEQ ID NO: 505. The open reading frame of the transgene can be seleted from any of the codon-optimized FVIII-QQ transgenes described herein, e.g., FVIII-QQ-01 to FVIII-QQ-18 (e.g., any of SEQ ID NOs 1, 2, 4, 5, 7-9, 11-15 or 1), where FVIII-SQ or FVIII-QQ-00 is used as a reference construct. Additionally, a FVIII-QQ construct as described herein can be made by changing the liver-specific promoter, e.g., HLP, of SEQ ID NO: 505. The liver-specific promoter can be seleted from any of the liver-specific promoters described herein, e.g., any of SEQ ID NOS: 86, 88, 91-96, 146-150, 439-441, or 481-500.

TABLE 12

| SEQ ID | | | |
|---|---|---|---|
| NO: | ELEMENT | LOCATION IN SEQ ID NO: 505 | TEXT WITHIN SEQ ID NO: 505 |
| 506 | HLP | 477-727 | italicized |
| 507 | Kozak sequence | 728-733 | bolded |
| 508 | F8SQ | 734-5,107 | double underlined |
| 509 | polyA | 5,108-5,156 | double underlined, bolded |
| 510 | L AAV2 ITRs | 347-476 | bolded, italicized |
| 511 | R AAV2 ITRs | 5,157-5,286 | bolded, italicized, underlined |
| 512 | origins of | 5,678-5,932 | dotted underlined, bolded |
| | replication | 7,569-7,823 | |
| 513 | Kanamicin | 6,538-6,792 | dotted underlined, bolded, |
| | resistance | | italicized |

While the present inventions have been described and illustrated in conjunction with a number of specific embodiments, those skilled in the art will appreciate that variations and modifications may be made without departing from the principles of the inventions as herein illustrated, as described and claimed. The present inventions may be embodied in other specific forms without departing from their spirit or essential characteristics. The described embodiments are considered in all respects to be illustrative and not restrictive.

In closing, regarding the exemplary embodiments of the present invention as shown and described herein, it will be appreciated that a genomic construct, comprising an AAV (adeno-associated virus) viral virion is disclosed and configured for delivery of AAV vectors. Because the principles of the invention may be practiced in a number of configurations beyond those shown and described, it is to be understood that the invention is not in any way limited by the exemplary embodiments, but is generally directed to a genomic construct, comprising an AAV (adeno-associated virus) viral virion apparatus and is able to take numerous forms to do so without departing from the spirit and scope of the invention.

Certain embodiments of the present invention are described herein, including the best mode known to the inventor(s) for carrying out the invention. Of course, variations on these described embodiments will become apparent to those of ordinary skill in the art upon reading the foregoing description. The inventor(s) expect skilled artisans to employ such variations as appropriate, and the inventor(s) intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein. Similarly, as used herein, unless indicated to the contrary, the term “substantially” is a term of degree intended to indicate an approximation of the characteristic, item, quantity, parameter, property, or term so qualified, encompassing a range that can be understood and construed by those of ordinary skill in the art.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

When used in the claims, whether as filed or added per amendment, the open-ended transitional term “comprising” (along with equivalent open-ended transitional phrases thereof such as “including,” “containing” and “having”) encompasses all the expressly recited elements, limitations, steps and/or features alone or in combination with un-recited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amendment for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim, whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (along with equivalent open-ended transitional phrases thereof) includes within its meaning, as a limiting case, claimed subject matter specified by the closed-ended transitional phrases “consisting of” or “consisting essentially of.” As such, embodiments described herein or so claimed with the phrase “comprising” are expressly or inherently unambiguously described, enabled and supported herein for the phrases “consisting essentially of” and “consisting of.”

While aspects of the invention have been described with reference to at least one exemplary embodiment, it is to be clearly understood by those skilled in the art that the invention is not limited thereto. Rather, the scope of the invention is to be interpreted only in conjunction with the appended claims and it is made clear, here, that the inventor(s) believe that the claimed subject matter is the invention.

The references disclosed in the specification and Examples, including but not limited to patents and patent applications, and international patent applications are all incorporated herein in their entirety by reference.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

TABLE 3
SEQUENCES

| SEQ | |
|---|---|
| ID | |
| NO: | SEQUENCE |
| 515 | FVIII-QQ00 |
| | ATGCAGATTGAGCTGAGCACCTGCTTCTTCCTGTGCCTGCTGAGGTTCTGCTTCTCTGCCACCAGGAGATACTACCTGGGGGCTGTGGAGCTGAGCTGGGACTACATGCAGTCTGACCTGGGGGAGCTGCCTGTGGATGCCAGGT |
| | TCCCCCCCAGAGTGCCCAAGAGCTTCCCCTTCAACACCTCTGTGGTGTACAAGAAGACCCTGTTTGTGGAGTTCACTGACCACCTGTTCAACATTGCCAAGCCCAGGCCCCCCTGGATGGGCCTGCTGGGCCCCACCATCCAGGC |
| | TGAGGTGTATGACACTGTGGTGATCACCCTGAAGAACATGGCCAGCCACCCTGTGAGCCTGCATGCTGTGGGGGTGAGCTACTGGAAGGCCTCTGAGGGGGCTGAGTATGATGACCAGACCAGCCAGAGGGAGAAGGAGGATGAC |
| | AAGGTGTTCCCTGGGGGCAGCCACACCTATGTGTGGCAGGTGCTGAAGGAGAATGGCCCCATGGCCTCTGACCCCCTGTGCCTGACCTACAGCTACCTGAGCCATGTGGACCTGGTGAAGGACCTGAACTCTGGCCTGATTGGGG |
| | CCCTGCTGGTGTGCAGGGAGGGCAGCCTGGCCAAGGAGAAGACCCAGACCCTGCACAAGTTCATCCTGCTGTTTGCTGTGTTTGATGAGGGCAAGAGCTGGCACTCTGAAACCAAGAACAGCCTGATGCAGGACAGGGATGCTGC |
| | CTCTGCCAGGGCCTGGCCCAAGATGCACACTGTGAATGGCTATGTGAACAGGAGCCTGCCTGGCCTGATTGGCTGCCACAGGAAGTCTGTGTACTGGCATGTGATTGGCATGGGCACCACCCCTGAGGTGCACAGCATCTTCCTG |
| | GAGGGCCACACCTTCCTGGTCAGGAACCACAGGCAGGCCAGCCTGGAGATCAGCCCCATCACCTTCCTGACTGCCCAGACCCTGCTGATGGACCTGGGCCAGTTCCTGCTGTTCTGCCACATCAGCAGCCACCAGCATGATGGCA |
| | TGGAGGCCTATGTGAAGGTGGACAGCTGCCCTGAGGAGCCCCAGCTGCAGATGAAGAACAATGAGGAGGCTGAGGACTATGATGATGACCTGACTGACTCTGAGATGGATGTGGTGAGGTTTGATGATGACAACAGCCCCAGCTT |
| | CATCCAGATCAGGTCTGTGGCCAAGAAGCACCCCAAGACCTGGGTGCACTACATTGCTGCTGAGGAGGAGGACTGGGACTATGCCCCCCTGGTGCTGGCCCCTGATGACAGGAGCTACAAGAGCCAGTACCTGAACAATGGCCCC |
| | CAGAGGATTGGCAGGAAGTACAAGAAGGTCAGGTTCATGGCCTACACTGATGAAACCTTCAAGACCAGGGAGGCCATCCAGCATGAGTCTGGCATCCTGGGCCCCCTGCTGTATGGGGAGGTGGGGGACACCCTGCTGATCATCT |
| | TCAAGAACCAGGCCAGCAGGCCCTACAACATCTACCCCCATGGCATCACTGATGTGAGGCCCCTGTACAGCAGGAGGCTGCCCAAGGGGGTGAAGCACCTGAAGGACTTCCCCATCCTGCCTGGGGAGATCTTCAAGTACAAGTG |
| | GACTGTGACTGTGGAGGATGGCCCCACCAAGTCTGACCCCAGGTGCCTGACCAGATACTACAGCAGCTTTGTGAACATGGAGAGGGACCTGGCCTCTGGCCTGATTGGCCCCCTGCTGATCTGCTACAAGGAGTCTGTGGACCAG |
| | CAGGGCAACCAGATCATGTCTGACAAGAGGAATGTGATCCTGTTCTCTGTGTTTGATGAGAACAGGAGCTGGTACCTGACTGAGAACATCCAGAGGTTCCTGCCCAACCCTGCTGGGGTGCAGCTGGAGGACCCTGAGTTCCAGG |
| | CCAGCAACATCATGCACAGCATCAATGGCTATGTGTTTGACAGCCTGCAGCTGTCTGTGTGCCTGCATGAGGTGGCCTACTGGTACATCCTGAGCATTGGGGCCCAGACTGACTTCCTGTCTGTGTTCTTCTCTGGCTACACCTT |
| | CAAGCACAAGATGGTGTATGAGGACACCCTGACCCTGTTCCCCTTCTCTGGGGAGACTGTGTTCATGAGCATGGAGAACCCTGGCCTGTGGATTCTGGGCTGCCACAACTCTGACTTCAGGAACAGGGGCATGACTGCCCTGCTG |
| | AAAGTCTCCAGCTGTGACAAGAACACTGGGGACTACTATGAGGACAGCTATGAGGACATCTCTGCCTACCTGCTGAGCAAGAACAATGCCATTGAGCCCAGGAGCTTCAGCCAGAACCCCCCAGTGCTGAAGAGGCACCAGAGGG |
| | AGATCACCAGGACCACCCTGCAGTCTGACCAGGAGGAGATTGACTATGATGACACCATCTCTGTGGAGATGAAGAAGGAGGACTTTGACATCTACGACGAGGACGAGAACCAGAGCCCCAGGAGCTTCCAGAAGAAGACCAGGCA |
| | CTACTTCATTGCTGCTGTGGAGAGGCTGTGGGACTATGGCATGAGCAGCAGCCCCCATGTGCTGAGGAACAGGGCCCAGTCTGGCTCTGTGCCCCAGTTCAAGAAGGTGGTGTTCCAGGAGTTCACTGATGGCAGCTTCACCCAG |
| | CCCCTGTACAGAGGGGAGCTGAATGAGCACCTGGGCCTGCTGGGCCCCTACATCAGGGCTGAGGTGGAGGACAACATCATGGTGACCTTCAGGAACCAGGCCAGCAGGCCCTACAGCTTCTACAGCAGCCTGATCAGCTATGAGG |
| | AGGACCAGAGGCAGGGGGCTGAGCCCAGGAAGAACTTTGTGAAGCCCAATGAAACCAAGACCTACTTCTGGAAGGTGCAGCACCACATGGCCCCCACCAAGGATGAGTTTGACTGCAAGGCCTGGGCCTACTTCTCTGATGTGGA |
| | CCTGGAGAAGGATGTGCACTCTGGCCTGATTGGCCCCCTGCTGGTGTGCCACACCAACACCCTGAACCCTGCCCATGGCAGGCAGGTGACTGTGCAGGAGTTTGCCCTGTTCTTCACCATCTTTGATGAAACCAAGAGCTGGTAC |
| | TTCACTGAGAACATGGAGAGGAACTGCAGGGCCCCCTGCAACATCCAGATGGAGGACCCCACCTTCAAGGAGAACTACAGGTTCCATGCCATCAATGGCTACATCATGGACACCCTGCCTGGCCTGGTGATGGCCCAGGACCAGA |
| | GGATCAGGTGGTACCTGCTGAGCATGGGCAGCAATGAGAACATCCACAGCATCCACTTCTCTGGCCATGTGTTCACTGTGAGGAAGAAGGAGGAGTACAAGATGGCCCTGTACAACCTGTACCCTGGGGTGTTTGAGACTGTGGA |
| | GATGCTGCCCAGCAAGGCTGGCATCTGGAGGGTGGAGTGCCTGATTGGGGAGCACCTGCATGCTGGCATGAGCACCCTGTTCCTGGTGTACAGCAACAAGTGCCAGACCCCCCTGGGCATGGCCTCTGGCCACATCAGGGACTTC |
| | CAGATCACTGCCTCTGGCCAGTATGGCCAGTGGGCCCCCAAGCTGGCCAGGCTGCACTACTCTGGCAGCATCAATGCCTGGAGCACCAAGGAGCCCTTCAGCTGGATCAAGGTGGACCTGCTGGCCCCCATGATCATCCATGGCA |
| | TCAAGACCCAGGGGGCCAGGCAGAAGTTCAGCAGCCTGTACATCAGCCAGTTCATCATCATGTACAGCCTGGATGGCAAGAAGTGGCAGACCTACAGGGGCAACAGCACTGGCACCCTGATGGTGTTCTTTGGCAATGTGGACAG |
| | CTCTGGCATCAAGCACAACATCTTCAACCCCCCCATCATTGCCAGATACATCAGGCTGCACCCCACCCACTACAGCATCAGGAGCACCCTGAGGATGGAGCTGATGGGCTGTGACCTGAACAGCTGCAGCATGCCCCTGGGCATG |
| | GAGAGCAAGGCCATCTCTGATGCCCAGATCACTGCCAGCAGCTACTTCACCAACATGTTTGCCACCTGGAGCCCCAGCAAGGCCAGGCTGCACCTGCAGGGCAGGAGCAATGCCTGGAGGCCCCAGGTCAACAACCCCAAGGAGT |
| | GGCTGCAGGTGGACTTCCAGAAGACCATGAAGGTGACTGGGGTGACCACCCAGGGGGTGAAGAGCCTGCTGACCAGCATGTATGTGAAGGAGTTCCTGATCAGCAGCAGCCAGGATGGCCACCAGTGGACCCTGTTCTTCCAGAA |
| | TGGCAAGGTGAAGGTGTTCCAGGGCAACCAGGACAGCTTCACCCCTGTGGTGAACAGCCTGGACCCCCCCCTGCTGACCAGATACCTGAGGATTCACCCCCAGAGCTGGGTGCACCAGATTGCCCTGAGGATGGAGGTGCTGGGC |
| | TGTGAGGCCCAGGACCTGTACTGA |
| 1 | FVIII-QQ01 |
| | ATGCAGATTGAGCTGAGCACCTGCTTCTTCCTGTGCCTGCTGAGATTCTGCTTCTCTGCCACCAGAAGATACTACCTGGGTGCAGTGGAGCTGAGCTGGGACTACATGCAGAGTGACCTGGGTGAGCTGCCTGTTGATGCCAGAT |
| | TCCCCCCCAGAGTGCCCAAGAGCTTCCCCTTCAACACCTCTGTTGTGTACAAGAAGACCCTGTTTGTTGAGAATTCACTGACCACCTGTTCAACATTGCCAAGCCCAGACCCCCCTGGATGGGCCTGCTGGGCCCCACCATCCAG |
| | GCAGAGGTGTATGATACTGTGGTGATCACCCTGAAGAACATGGCCAGCCACCCAGTTAGCCTGCATGCAGTGGGGGTCAGCTACTGGAAGGCCTCAGAAGGGGCTGAGTATGATGATCAGACCAGCCAGAGAGAGAAGGAGGATG |
| | ACAAGGTGTTCCCAGGGGGTAGCCACACCTATGTATGGCAGGTGCTGAAGGAGAATGGCCCCATGGCCTCTGACCCCCTGTGCCTGACCTACAGCTACCTGAGCCATGTGGACCTGGTGAAGGACCTGAACAGTGGACTGATTGG |
| | TGCCCTGCTGGTGTGCAGAGAGGGCAGCCTGGCCAAGGAGAAGACCCAGACCCTGCACAAGTTCATCCTGCTGTTTGCTGTGTTTGATGAAGGCAAGAGCTGGCACAGTGAGACCAAGAACAGCCTGATGCAGGACAGAGATGCT |
| | GCATCAGCTAGAGCCTGGCCCAAGATGCACACTGTCAATGGTTATGTAAACAGAAGCCTGCCTGGCCTGATTGGCTGCCACAGAAAGTCAGTATACTGGCATGTTATAGGCATGGGCACCACCCCAGAGGTGCACAGCATCTTCC |
| | TGGAGGGCCACACCTTCCTGGTGAGAAACCACAGACAGGCCAGCCTGGAGATCAGCCCCATCACCTTCCTGACAGCCCAGACCCTGCTGATGGACCTGGGCCAGTTCCTGCTGTTCTGCCACATCAGCAGCCACCAGCATGATGG |
| | GATGGAGGCCTATGTAAAGGTGGACAGCTGCCCAGAGGAGCCCCAGCTGCAGATGAAGAACAATGAAGAGGCTGAAGACTATGATGATGATCTGACTGATTCAGAGATGGATGTTGTGAGATTTGATGATGACAACAGCCCCAGC |
| | TTCATCCAGATCAGATCAGTGGCCAAGAAGCACCCCAAGACCTGGGTGCACTACATAGCTGCAGAGGAGGAGGACTGGGACTATGCCCCCCTGGTGCTGGCCCCAGATGATAGAAGCTACAAGAGCCAGTACCTGAACAATGGCC |
| | CCCAGAGAATAGGGAGAAAGTACAAGAAGGTGAGATTCATGGCCTACACAGATGAGACCTTCAAGACCAGAGAGGCCATCCAGCATGAGTCAGGTATCCTGGGCCCCCTGCTGTATGGGGAGGTGGGTGACACCCTGCTGATCAT |
| | CTTCAAGAACCAGGCCAGCAGACCCTACAACATCTACCCCCATGGGATCACAGATGTCAGACCCCTGTACAGCAGAAGACTGCCCAAGGGAGTCAAGCACCTGAAGGACTTCCCCATCCTGCCTGGAGAAATCTTCAAGTACAAG |
| | TGGACAGTGACTGTGGAGGATGGTCCCACCAAGAGTGACCCCAGATGCCTGACCAGATACTACAGCAGCTTTGTCAACATGGAGAGAGACCTGGCCAGTGGCCTGATAGGCCCCCTGCTGATCTGCTACAAGGAGTCTGTGGACC |
| | AGCAGGGCAACCAGATCATGTCTGACAAGAGAAATGTTATCCTGTTCAGTGTATTTGATGAGAACAGAAGCTGGTACCTGACTGAGAACATCCAGAGATTCCTGCCCAACCCAGCTGGGGTCCAGCTGGAGGACCCTGAGTTCCA |
| | GGCCAGCAACATCATGCACAGCATCAATGGCTATGTGTTTGACAGCCTGCAGCTGTCTGTCTGCCTGCATGAAGTGGCCTACTGGTACATCCTGAGCATAGGAGCCCAGACAGACTTCCTGAGTGTATTCTTCTCTGGCTACACC |
| | TTCAAGCACAAGATGGTGTATGAGGACACCCTGACCCTGTTCCCCTTCTCTGGAGAGACTGTATTCATGAGCATGGAGAACCCTGGCCTGTGGATCCTGGGCTGCCACAACTCTGACTTCAGAAACAGAGGCATGACTGCCCTGC |
| | TGAAGGTGAGCAGCTGTGATAAGAACACAGGGGACTACTATGAGGACAGCTATGAGGACATCTCAGCCTACCTGCTGAGCAAGAACAATGCCATTGAGCCCAGAAGCTTCAGCCAGAACCCCCCTGTGCTGAAGAGACACCAGAG |
| | AGAGATCACCAGAACCACCCTGCAGAGTGACCAGGAGGAGATTGACTATGATGACACCATCTCTGTTGAGATGAAGAAGGAGGACTTTGACATCTATGATGAGGATGAGAACCAGAGCCCCAGAAGCTTCCAGAAGAAGACCAGA |
| | CACTACTTCATTGCAGCTGTGGAGAGACTGTGGGACTATGGCATGAGCAGCAGCCCCCATGTGCTGAGAAACAGAGCCCAGTCAGGCTCTGTGCCCCAGTTCAAGAAGGTGGTGTTCCAGGAGTTCACAGATGGGAGCTTCACCC |
| | AGCCCCTGTACAGAGGTGAACTGAATGAGCACCTGGGCCTGCTGGGCCCCTACATCAGAGCTGAAGTGGAGGACAACATCATGGTGACCTTCAGAAACCAGGCCAGCAGACCCTACAGCTTCTACAGCAGCCTGATCAGCTATGA |
| | AGAGGACCAGAGACAGGGTGCTGAGCCCAGAAAGAACTTTGTGAAGCCCAATGAAACCAAGACCTACTTCTGGAAGGTGCAGCACCACATGGCCCCCACCAAGGATGAATTTGACTGCAAGGCCTGGGCCTACTTCTCAGATGTA |
| | GACCTGGAGAAGGATGTTCACTCAGGCCTGATTGGGCCCCTGCTGGTGTGCCACACCAACACCCTGAACCCAGCCCATGGTAGACAGGTGACAGTGCAGGAGTTTGCCCTGTTCTTCACCATCTTTGATGAGACCAAGAGCTGGT |
| | ACTTCACTGAGAACATGGAGAGAAACTGCAGAGCCCCCTGCAACATCCAGATGGAGGACCCCACCTTCAAGGAGAACTACAGATTCCATGCCATCAATGGGTACATCATGGACACCCTGCCAGGCCTGGTGATGGCCCAGGACCA |
| | GAGAATCAGATGGTACCTGCTGAGCATGGGCAGCAATGAGAACATCCACAGCATCCACTTCAGTGGTCATGTCTTCACAGTGAGAAAGAAGGAGGAGTACAAGATGGCCCTGTACAACCTGTACCCAGGGGTGTTTGAAACTGTA |
| | GAGATGCTGCCCAGCAAGGCAGGCATCTGGAGAGTGGAGTGCCTGATAGGAGAACACCTGCATGCAGGAATGAGCACCCTGTTCCTGGTGTACAGCAACAAGTGCCAGACCCCCCTGGGCATGGCCTCTGGGCACATCAGAGACT |
| | TCCAGATCACTGCCTCTGGCCAGTATGGGCAGTGGGCCCCCAAGCTGGCCAGACTGCACTACTCAGGTAGCATCAATGCCTGGAGCACCAAGGAGCCCTTCAGCTGGATCAAGGTGGACCTGCTGGCCCCCATGATCATCCATGG |
| | TATCAAGACCCAGGGTGCCAGACAGAAGTTCAGCAGCCTGTACATCAGCCAGTTCATCATCATGTACAGCCTGGATGGCAAGAAGTGGCAGACCTACAGAGGCAACAGCACTGGCACCCTGATGGTGTTCTTTGGCAATGTGGAC |
| | AGCTCTGGCATCAAGCACAACATCTTCAACCCCCCCATCATAGCCAGATACATCAGACTGCACCCCACCCACTACAGCATCAGAAGCACCCTGAGAATGGAGCTGATGGGCTGTGATCTGAACAGCTGCAGCATGCCCCTGGGCA |
| | TGGAGAGCAAGGCCATCTCTGATGCTCAGATCACTGCAAGCAGCTACTTCACCAACATGTTTGCCACCTGGAGCCCCAGCAAGGCCAGACTGCACCTGCAGGGCAGAAGCAATGCCTGGAGACCCCAGGTGAACAACCCCAAGGA |
| | GTGGCTGCAGGTGGACTTCCAGAAGACCATGAAGGTGACAGGTGTCACCACCCAGGGGGTGAAGAGCCTGCTGACCAGCATGTATGTGAAGGAGTTCCTGATCAGCAGCAGCCAGGATGGCCACCAGTGGACCCTGTTCTTCCAG |
| | AATGGCAAGGTGAAGGTGTTCCAGGGCAACCAGGACAGCTTCACCCCTGTGGTGAACAGCCTGGACCCCCCCCTGCTGACCAGATACCTGAGAATCCACCCCCAGAGCTGGGTGCACCAGATTGCCCTGAGAATGGAGGTGCTGG |
| | GCTGTGAGGCCCAGGACCTGTACTGA |
| 2 | FVIII-QQ02 |
| | ATGCAGATTGAGCTGAGCACCTGCTTCTTCCTGTGCCTGCTGAGATTCTGCTTCAGTGCCACCAGAAGATACTACCTGGGAGCAGTGGAGCTGAGCTGGGACTACATGCAGAGTGATCTGGGAGAGCTGCCAGTAGATGCCAGAT |
| | TCCCCCCCAGAGTGCCCAAGAGCTTCCCCTTCAACACCTCAGTGGTGTACAAGAAGACCCTGTTTGTTGAGTTCACTGACCACCTGTTCAACATTGCAAAGCCCAGACCCCCCTGGATGGGCCTGCTGGGCCCCACCATCCAGGC |
| | AGAAGTGTATGACACAGTAGTGATCACCCTGAAGAACATGGCCAGCCACCCTGTCAGCCTGCATGCAGTTGGTGTAAGCTACTGGAAGGCCTCTGAGGGTGCTGAATATGATGACCAGACCAGCCAGAGAGAGAAGGAGGATGAT |
| | AAGGTGTTCCCAGGGGGCAGCCACACCTATGTTTGGCAGGTGCTGAAGGAGAATGGGCCCATGGCCTCTGATCCCCTGTGCCTGACCTACAGCTACCTGAGCCATGTGGACCTGGTGAAGGACCTGAACTCTGGGCTGATAGGAG |
| | CCCTGCTGGTGTGCAGAGAGGGCAGCCTGGCCAAGGAGAAGACCCAGACCCTGCACAAGTTCATCCTGCTGTTTGCTGTGTTTGATGAAGGCAAGAGCTGGCACAGTGAAACCAAGAACAGCCTGATGCAGGACAGAGATGCAGC |
| | TAGTGCCAGAGCCTGGCCCAAGATGCACACAGTAAATGGTTATGTGAACAGAAGCCTGCCTGGCCTGATAGGCTGCCACAGAAAGTCTGTGTACTGGCATGTTATAGGTATGGGCACCACCCCTGAGGTGCACAGCATCTTCCTG |
| | GAGGGCCACACCTTCCTGGTGAGAAACCACAGACAGGCCAGCCTGGAGATCAGCCCCATCACCTTCCTGACTGCCCAGACCCTGCTGATGGACCTGGGCCAGTTCCTGCTGTTCTGCCACATCAGCAGCCACCAGCATGATGGAA |
| | TGGAGGCCTATGTCAAGGTGGACAGCTGCCCAGAGGAGCCCCAGCTGCAGATGAAGAACAATGAGGAGGCAGAGGACTATGATGATGACCTGACTGACAGTGAAATGGATGTGGTCAGATTTGATGATGATAACAGCCCCAGCTT |
| | CATCCAGATCAGATCAGTGGCCAAGAAGCACCCCAAGACCTGGGTGCACTACATTGCAGCTGAGGAGGAGGACTGGGACTATGCACCCCTGGTGCTGGCCCCAGATGATAGAAGCTACAAGAGCCAGTACCTGAACAATGGACCC |
| | CAGAGAATTGGCAGAAAGTACAAGAAGGTGAGATTCATGGCCTACACTGATGAGACCTTCAAGACCAGAGAGGCCATCCAGCATGAATCAGGCATCCTGGGCCCCCTGCTGTATGGGGAGGTGGGAGACACCCTGCTGATCATCT |
| | TCAAGAACCAGGCCAGCAGACCCTACAACATCTACCCCCATGGAATCACAGATGTTAGACCCCTGTACAGCAGAAGACTGCCCAAGGGGGTGAAGCACCTGAAGGACTTCCCCATCCTGCCTGGGGAGATCTTCAAGTACAAGTG |
| | GACAGTTACAGTAGAGGATGGCCCCACCAAGAGTGACCCCAGATGCCTGACCAGATACTACAGCAGCTTTGTAAACATGGAGAGAGACCTGGCCTCTGGCCTGATAGGCCCCCTGCTGATCTGCTACAAGGAGTCAGTTGACCAG |
| | CAGGGCAACCAGATCATGAGTGATAAGAGAAATGTCATCCTGTTCAGTGTGTTTGATGAGAACAGAAGCTGGTACCTGACAGAGAACATCCAGAGATTCCTGCCCAACCCTGCTGGAGTGCAGCTGGAGGACCCAGAATTCCAGG |
| | CCAGCAACATCATGCACAGCATCAATGGTTATGTCTTTGATAGCCTGCAGCTGTCAGTTTGCCTGCATGAGGTGGCCTACTGGTACATCCTGAGCATTGGGGCCCAGACTGACTTCCTGAGTGTATTCTTCAGTGGCTACACCTT |
| | CAAGCACAAGATGGTGTATGAGGACACCCTGACCCTGTTCCCCTTCTCTGGAGAGACTGTGTTCATGAGCATGGAGAACCCTGGGCTGTGGATCCTGGGCTGCCACAACTCAGACTTCAGAAACAGAGGCATGACAGCACTGCTG |
| | AAGGTGAGCAGCTGTGATAAGAACACTGGTGACTACTATGAGGACAGCTATGAAGACATCTCAGCCTACCTGCTGAGCAAGAACAATGCCATAGAGCCCAGAAGCTTCAGCCAGAACCCCCCAGTTCTGAAGAGACACCAGAGAG |
| | AGATCACCAGAACCACCCTGCAGAGTGACCAGGAGGAGATAGATTATGATGATACCATCTCAGTTGAGATGAAGAAGGAGGACTTTGATATCTATGATGAGGATGAGAACCAGAGCCCCAGAAGCTTCCAGAAGAAGACCAGACA |
| | CTACTTCATAGCAGCTGTGGAGAGACTGTGGGACTATGGCATGAGCAGCAGCCCCCATGTTCTGAGAAACAGAGCCCAGAGTGGATCAGTACCCCAGTTCAAGAAGGTGGTGTTCCAGGAGTTCACTGATGGGAGCTTCACCCAG |
| | CCCCTGTACAGAGGTGAACTGAATGAGCACCTGGGCCTGCTGGGCCCCTACATCAGAGCTGAAGTGGAGGACAACATCATGGTGACCTTCAGAAACCAGGCCAGCAGACCCTACAGCTTCTACAGCAGCCTGATCAGCTATGAGG |
| | AGGACCAGAGACAGGGAGCTGAGCCCAGAAAGAACTTTGTCAAGCCCAATGAAACCAAGACCTACTTCTGGAAGGTGCAGCACCACATGGCCCCCACCAAGGATGAGTTTGACTGCAAGGCCTGGGCCTACTTCAGTGATGTAGA |
| | CCTGGAGAAGGATGTCCACTCAGGCCTGATTGGCCCCCTGCTGGTGTGCCACACCAACACCCTGAACCCAGCACATGGGAGACAGGTGACTGTACAGGAGTTTGCTCTGTTCTTCACCATCTTTGATGAGACCAAGAGCTGGTAC |
| | TTCACAGAAAACATGGAGAGAAACTGCAGAGCCCCCTGCAACATCCAGATGGAGGACCCCACCTTCAAGGAGAACTACAGATTCCATGCCATCAATGGTTACATCATGGACACCCTGCCTGGCCTGGTGATGGCCCAGGACCAGA |
| | GAATCAGATGGTACCTGCTGAGCATGGGCAGCAATGAAAACATCCACAGCATCCACTTCTCAGGGCATGTGTTCACTGTGAGAAAGAAGGAGGAGTACAAGATGGCCCTGTACAACCTGTACCCAGGTGTGTTTGAAACTGTGGA |
| | GATGCTGCCCAGCAAGGCAGGCATCTGGAGAGTGGAGTGCCTGATAGGGGAGCACCTGCATGCAGGGATGAGCACCCTGTTCCTGGTGTACAGCAACAAGTGCCAGACCCCCCTGGGCATGGCCTCAGGCCACATCAGAGACTTC |
| | CAGATCACAGCTTCTGGACAGTATGGCCAGTGGGCCCCCAAGCTGGCCAGACTGCACTACAGTGGCAGCATCAATGCCTGGAGCACCAAGGAGCCCTTCAGCTGGATCAAGGTGGACCTGCTGGCCCCCATGATCATCCATGGCA |
| | TCAAGACCCAGGGAGCCAGACAGAAGTTCAGCAGCCTGTACATCAGCCAGTTCATCATCATGTACAGCCTGGATGGCAAGAAGTGGCAGACCTACAGAGGCAACAGCACAGGCACCCTGATGGTGTTCTTTGGGAATGTGGACAG |
| | CAGTGGAATCAAGCACAACATCTTCAACCCCCCCATCATTGCAAGATACATCAGACTGCACCCCACCCACTACAGCATCAGAAGCACCCTGAGAATGGAGCTGATGGGCTGTGACCTGAACAGCTGCAGCATGCCCCTGGGCATG |
| | GAGAGCAAGGCCATCTCTGATGCCCAGATCACAGCTAGCAGCTACTTCACCAACATGTTTGCCACCTGGAGCCCCAGCAAGGCCAGACTGCACCTGCAGGGCAGAAGCAATGCCTGGAGACCCCAGGTGAACAACCCCAAGGAGT |
| | GGCTGCAGGTGGACTTCCAGAAGACCATGAAGGTGACTGGTGTTACCACCCAGGGGGTCAAGAGCCTGCTGACCAGCATGTATGTGAAGGAGTTCCTGATCAGCAGCAGCCAGGATGGCCACCAGTGGACCCTGTTCTTCCAGAA |
| | TGGCAAGGTGAAGGTGTTCCAGGGCAACCAGGACAGCTTCACCCCTGTGGTGAACAGCCTGGACCCCCCCCTGCTGACCAGATACCTGAGAATCCACCCCCAGAGCTGGGTGCACCAGATAGCACTGAGAATGGAGGTGCTGGGC |
| | TGTGAGGCCCAGGACCTGTACTGA |
| 3 | FVIII-QQ03 |
| | ATGCAAATTGAGTTGTCCACCTGCTTCTTCTTGTGCTTGTTGAGGTTCTGCTTCAGTGCCACCAGGAGGTACTACTTGGGGGCTGTGGAGTTGTCCTGGGACTACATGCAGTCAGACTTGGGGGAGTTGCCTGTGGATGCCAGGT |
| | TCCCCCCCAGGGTCCCCAAGTCCTTCCCCTTCAACACCAGTGTGGTCTACAAGAAGACCTTGTTTGTGGAGTTCACTGACCACTTGTTCAACATTGCCAAGCCCAGGCCCCCCTGGATGGGCTTGTTGGGCCCCACCATCCAGGC |
| | TGAGGTCTATGACACAGTGGTCATCACCTTGAAGAACATGGCCTCCCACCCTGTCTCCTTGCATGCAGTGGGGGTCTCCTACTGGAAGGCCAGTGAGGGGGCTGAGTATGATGACCAGACCTCCCAGAGGGAGAAGGAGGATGAC |
| | AAGGTCTTCCCTGGGGGCTCCCACACCTATGTCTGGCAGGTCTTGAAGGAGAATGGCCCCATGGCCTCTGACCCCTTGTGCTTGACCTACTCCTACTTGTCCCATGTGGACTTGGTCAAGGACTTGAACTCAGGCTTGATTGGGG |
| | CCTTGTTGGTCTGCAGGGAGGGCTCCTTGGCCAAGGAGAAGACCCAGACCTTGCACAAGTTCATCTTGTTGTTTGCAGTCTTTGATGAGGGCAAGTCCTGGCACAGTGAGACCAAGAACTCCTTGATGCAGGACAGGGATGCTGC |
| | CAGTGCCAGGGCCTGGCCCAAGATGCACACTGTCAATGGCTATGTCAACAGGTCCTTGCCTGGCTTGATTGGCTGCCACAGGAAGTCAGTCTACTGGCATGTCATTGGCATGGGCACCACCCCTGAGGTCCACTCCATCTTCTTG |
| | GAGGGCCACACCTTCTTGGTCAGGAACCACAGGCAGGCCTCCTTGGAGATCTCCCCCATCACCTTCTTGACAGCCCAGACCTTGTTGATGGACTTGGGCCAGTTCTTGTTGTTCTGCCACATCTCCTCCCACCAGCATGATGGCA |
| | TGGAGGCCTATGTCAAGGTGGACTCCTGCCCTGAGGAGCCCCAGTTGCAGATGAAGAACAATGAGGAGGCAGAGGACTATGATGATGACTTGACTGACTCTGAGATGGATGTGGTCAGGTTTGATGATGACAACTCCCCCTCCTT |
| | CATCCAGATCAGGAGTGTGGCCAAGAAGCACCCCAAGACCTGGGTCCACTACATAGCAGCAGAGGAGGAGGACTGGGACTATGCCCCCTTGGTCTTGGCCCCTGATGACAGGTCCTACAAGTCCCAGTACTTGAACAATGGCCCC |
| | CAGAGGATTGGCAGGAAGTACAAGAAGGTCAGGTTCATGGCCTACACAGATGAGACCTTCAAGACCAGGGAGGCCATCCAGCATGAGTCAGGCATCTTGGGCCCCTTGTTGTATGGGGAGGTGGGGGACACCTTGTTGATCATCT |
| | TCAAGAACCAGGCCTCCAGGCCCTACAACATCTACCCCCATGGCATCACTGATGTCAGGCCCTTGTACTCCAGGAGGTTGCCCAAGGGGGTCAAGCACTTGAAGGACTTCCCCATCTTGCCAGGGGAGATCTTCAAGTACAAGTG |
| | GACAGTCACTGTGGAGGATGGCCCCACCAAGTCAGACCCCAGGTGCTTGACCAGGTACTACTCCTCCTTTGTCAACATGGAGAGGGACTTGGCCAGTGGCTTGATTGGCCCCTTGTTGATCTGCTACAAGGAGTCTGTGGACCAG |
| | CAGGGCAACCAGATCATGTCAGACAAGAGGAATGTCATCTTGTTCAGTGTCTTTGATGAGAACAGGTCCTGGTACTTGACAGAGAACATCCAGAGGTTCTTGCCCAACCCAGCTGGGGTCCAGTTGGAGGACCCTGAGTTCCAGG |
| | CCTCCAACATCATGCACTCCATCAATGGCTATGTCTTTGACTCCTTGCAGTTGTCAGTCTGCTTGCATGAGGTGGCCTACTGGTACATCTTGTCCATTGGGGCCCAGACTGACTTCTTGTCTGTCTTCTTCAGTGGCTACACCTT |
| | CAAGCACAAGATGGTCTATGAGGACACCTTGACCTTGTTCCCCTTCAGTGGGGAGACTGTCTTCATGTCCATGGAGAACCCAGGCTTGTGGATCTTGGGCTGCCACAACTCAGACTTCAGGAACAGGGGCATGACAGCCTTGTTG |
| | AAGGTCTCCTCCTGTGACAAGAACACAGGGGACTACTATGAGGACTCCTATGAGGACATCTCAGCCTACTTGTTGTCCAAGAACAATGCCATAGAGCCCAGGTCCTTCTCCCAGAACCCCCCTGTCTTGAAGAGGCACCAGAGGG |
| | AGATCACCAGGACCACCTTGCAGTCAGACCAGGAGGAGATAGACTATGATGACACCATCTCAGTGGAGATGAAGAAGGAGGACTTTGACATCTATGATGAGGATGAGAACCAGTCCCCCAGGTCCTTCCAGAAGAAGACCAGGCA |
| | CTACTTCATTGCAGCAGTGGAGAGGTTGTGGGACTATGGCATGTCCTCCTCCCCCCATGTCTTGAGGAACAGGGCCCAGTCAGGCAGTGTCCCCCAGTTCAAGAAGGTGGTCTTCCAGGAGTTCACTGATGGCTCCTTCACCCAG |
| | CCCTTGTACAGGGGGGAGTTGAATGAGCACTTGGGCTTGTTGGGCCCCTACATCAGGGCTGAGGTGGAGGACAACATCATGGTCACCTTCAGGAACCAGGCCTCCAGGCCCTACTCCTTCTACTCCTCCTTGATCTCCTATGAGG |
| | AGGACCAGAGGCAGGGGGCAGAGCCCAGGAAGAACTTTGTCAAGCCCAATGAGACCAAGACCTACTTCTGGAAGGTCCAGCACCACATGGCCCCCACCAAGGATGAGTTTGACTGCAAGGCCTGGGCCTACTTCTCTGATGTGGA |
| | CTTGGAGAAGGATGTCCACTCTGGCTTGATTGGCCCCTTGTTGGTCTGCCACACCAACACCTTGAACCCTGCCCATGGCAGGCAGGTCACTGTCCAGGAGTTTGCCTTGTTCTTCACCATCTTTGATGAGACCAAGTCCTGGTAC |
| | TTCACAGAGAACATGGAGAGGAACTGCAGGGCCCCCTGCAACATCCAGATGGAGGACCCCACCTTCAAGGAGAACTACAGGTTCCATGCCATCAATGGCTACATCATGGACACCTTGCCAGGCTTGGTCATGGCCCAGGACCAGA |
| | GGATCAGGTGGTACTTGTTGTCCATGGGCTCCAATGAGAACATCCACTCCATCCACTTCAGTGGCCATGTCTTCACTGTCAGGAAGAAGGAGGAGTACAAGATGGCCTTGTACAACTTGTACCCAGGGGTCTTTGAGACAGTGGA |
| | GATGTTGCCCTCCAAGGCAGGCATCTGGAGGGTGGAGTGCTTGATTGGGGAGCACTTGCATGCTGGCATGTCCACCTTGTTCTTGGTCTACTCCAACAAGTGCCAGACCCCCTTGGGCATGGCCTCTGGCCACATCAGGGACTTC |
| | CAGATCACTGCCTCAGGCCAGTATGGCCAGTGGGCCCCCAAGTTGGCCAGGTTGCACTACAGTGGCTCCATCAATGCCTGGTCCACCAAGGAGCCCTTCTCCTGGATCAAGGTGGACTTGTTGGCCCCCATGATCATCCATGGCA |
| | TCAAGACCCAGGGGGCCAGGCAGAAGTTCTCCTCCTTGTACATCTCCCAGTTCATCATCATGTACTCCTTGGATGGCAAGAAGTGGCAGACCTACAGGGGCAACTCCACAGGCACCTTGATGGTCTTCTTTGGCAATGTGGACTC |
| | CTCTGGCATCAAGCACAACATCTTCAACCCCCCCATCATTGCCAGGTACATCAGGTTGCACCCCACCCACTACTCCATCAGGTCCACCTTGAGGATGGAGTTGATGGGCTGTGACTTGAACTCCTGCTCCATGCCCTTGGGCATG |
| | GAGTCCAAGGCCATCTCAGATGCCCAGATCACTGCCTCCTCCTACTTCACCAACATGTTTGCCACCTGGTCCCCCTCCAAGGCCAGGTTGCACTTGCAGGGCAGGTCCAATGCCTGGAGGCCCCAGGTCAACAACCCCAAGGAGT |
| | GGTTGCAGGTGGACTTCCAGAAGACCATGAAGGTCACAGGGGTCACCACCCAGGGGGTCAAGTCCTTGTTGACCTCCATGTATGTCAAGGAGTTCTTGATCTCCTCCTCCCAGGATGGCCACCAGTGGACCTTGTTCTTCCAGAA |
| | TGGCAAGGTCAAGGTCTTCCAGGGCAACCAGGACTCCTTCACCCCTGTGGTCAACTCCTTGGACCCCCCCTTGTTGACCAGGTACTTGAGGATCCACCCCCAGTCCTGGGTCCACCAGATTGCCTTGAGGATGGAGGTCTTGGGC |
| | TGTGAGGCCCAGGACTTGTACTGA |
| 4 | FVIII-QQ04 |
| | ATGCAGATAGAGCTGAGCACCTGCTTCTTCCTGTGCCTGCTGAGATTCTGCTTCTCAGCCACCAGAAGATACTACCTGGGGGCTGTAGAGCTGAGCTGGGACTACATGCAGTCAGACCTGGGTGAGCTGCCAGTGGATGCCAGAT |
| | TCCCCCCCAGAGTGCCCAAGAGCTTCCCCTTCAACACCTCTGTGGTGTACAAGAAGACCCTGTTTGTGGAGTTCACTGATCACCTGTTCAACATAGCTAAGCCCAGACCCCCCTGGATGGGCCTGCTGGGCCCCACCATCCAGGC |
| | TGAGGTGTATGATACAGTGGTGATCACCCTGAAGAACATGGCCAGCCACCCTGTGAGCCTGCATGCAGTGGGTGTGAGCTACTGGAAGGCCTCTGAGGGTGCTGAATATGATGACCAGACCAGCCAGAGAGAGAAGGAGGATGAT |
| | AAGGTGTTCCCTGGGGGCAGCCACACCTATGTGTGGCAGGTGCTGAAGGAGAATGGACCCATGGCCAGTGACCCCCTGTGCCTGACCTACAGCTACCTGAGCCATGTAGACCTGGTGAAGGACCTGAACTCTGGACTGATTGGGG |
| | CACTGCTGGTGTGCAGAGAGGGCAGCCTGGCCAAGGAGAAGACCCAGACCCTGCACAAGTTCATCCTGCTGTTTGCTGTGTTTGATGAGGGCAAGAGCTGGCACTCTGAAACCAAGAACAGCCTGATGCAGGACAGAGATGCTGC |
| | CTCAGCAAGAGCCTGGCCCAAGATGCACACTGTGAATGGCTATGTTAACAGAAGCCTGCCTGGGCTGATTGGGTGCCACAGAAAGAGTGTGTACTGGCATGTAATAGGAATGGGCACCACCCCAGAGGTGCACAGCATCTTCCTG |
| | GAGGGCCACACCTTCCTGGTGAGAAACCACAGACAGGCCAGCCTGGAGATCAGCCCCATCACCTTCCTGACTGCTCAGACCCTGCTGATGGACCTGGGCCAGTTCCTGCTGTTCTGCCACATCAGCAGCCACCAGCATGATGGAA |
| | TGGAGGCCTATGTAAAGGTGGACAGCTGCCCTGAGGAGCCCCAGCTGCAGATGAAGAACAATGAGGAGGCAGAGGACTATGATGATGACCTGACTGACAGTGAGATGGATGTGGTGAGATTTGATGATGATAACAGCCCCAGCTT |
| | CATCCAGATCAGATCTGTAGCCAAGAAGCACCCCAAGACCTGGGTGCACTACATAGCTGCAGAAGAGGAGGACTGGGACTATGCCCCCCTGGTGCTGGCCCCTGATGATAGAAGCTACAAGAGCCAGTACCTGAACAATGGTCCC |
| | CAGAGAATAGGGAGAAAGTACAAGAAGGTGAGATTCATGGCCTACACTGATGAGACCTTCAAGACCAGAGAGGCCATCCAGCATGAGTCAGGCATCCTGGGCCCCCTGCTGTATGGAGAAGTGGGTGACACCCTGCTGATCATCT |
| | TCAAGAACCAGGCCAGCAGACCCTACAACATCTACCCCCATGGCATCACTGATGTCAGACCCCTGTACAGCAGAAGACTGCCCAAGGGTGTGAAGCACCTGAAGGACTTCCCCATCCTGCCAGGTGAGATCTTCAAGTACAAGTG |
| | GACAGTGACTGTAGAGGATGGGCCCACCAAGTCAGACCCCAGATGCCTGACCAGATACTACAGCAGCTTTGTGAACATGGAGAGAGACCTGGCCAGTGGCCTGATTGGCCCCCTGCTGATCTGCTACAAGGAGTCAGTTGACCAG |
| | CAGGGCAACCAGATCATGTCTGACAAGAGAAATGTCATCCTGTTCAGTGTGTTTGATGAGAACAGAAGCTGGTACCTGACTGAGAACATCCAGAGATTCCTGCCCAACCCAGCAGGAGTTCAGCTGGAGGACCCTGAGTTCCAGG |
| | CCAGCAACATCATGCACAGCATCAATGGCTATGTTTTTGACAGCCTGCAGCTGTCAGTGTGCCTGCATGAAGTGGCCTACTGGTACATCCTGAGCATTGGTGCCCAGACAGATTTCCTGAGTGTCTTCTTCTCAGGCTACACCTT |
| | CAAGCACAAGATGGTGTATGAGGACACCCTGACCCTGTTCCCCTTCTCTGGGGAAACAGTGTTCATGAGCATGGAGAACCCTGGTCTGTGGATCCTGGGCTGCCACAACTCAGACTTCAGAAACAGAGGCATGACAGCACTGCTG |
| | AAGGTGAGCAGCTGTGACAAGAACACAGGTGACTACTATGAAGACAGCTATGAGGACATCTCAGCCTACCTGCTGAGCAAGAACAATGCCATTGAGCCCAGAAGCTTCAGCCAGAACCCCCCTGTGCTGAAGAGACACCAGAGAG |
| | AGATCACCAGAACCACCCTGCAGTCAGACCAGGAGGAGATTGACTATGATGACACCATCAGTGTAGAAATGAAGAAGGAGGACTTTGACATCTATGATGAGGATGAAAACCAGAGCCCCAGAAGCTTCCAGAAGAAGACCAGACA |
| | CTACTTCATAGCTGCAGTTGAGAGACTGTGGGACTATGGAATGAGCAGCAGCCCCCATGTGCTGAGAAACAGAGCCCAGTCTGGCTCAGTTCCCCAGTTCAAGAAGGTGGTGTTCCAGGAGTTCACAGATGGGAGCTTCACCCAG |
| | CCCCTGTACAGAGGGGAGCTGAATGAGCACCTGGGCCTGCTGGGCCCCTACATCAGAGCAGAGGTGGAGGACAACATCATGGTGACCTTCAGAAACCAGGCCAGCAGACCCTACAGCTTCTACAGCAGCCTGATCAGCTATGAAG |
| | AGGACCAGAGACAGGGAGCAGAACCCAGAAAGAACTTTGTTAAGCCCAATGAAACCAAGACCTACTTCTGGAAGGTGCAGCACCACATGGCCCCCACCAAGGATGAATTTGACTGCAAGGCCTGGGCCTACTTCTCTGATGTTGA |
| | CCTGGAGAAGGATGTGCACTCAGGCCTGATAGGCCCCCTGCTGGTGTGCCACACCAACACCCTGAACCCAGCTCATGGGAGACAGGTGACTGTTCAGGAGTTTGCCCTGTTCTTCACCATCTTTGATGAGACCAAGAGCTGGTAC |
| | TTCACAGAGAACATGGAGAGAAACTGCAGAGCCCCCTGCAACATCCAGATGGAGGACCCCACCTTCAAGGAGAACTACAGATTCCATGCCATCAATGGTTACATCATGGACACCCTGCCTGGGCTGGTGATGGCCCAGGACCAGA |
| | GAATCAGATGGTACCTGCTGAGCATGGGCAGCAATGAAAACATCCACAGCATCCACTTCTCTGGCCATGTGTTCACTGTCAGAAAGAAGGAGGAGTACAAGATGGCCCTGTACAACCTGTACCCTGGAGTATTTGAGACTGTGGA |
| | GATGCTGCCCAGCAAGGCAGGTATCTGGAGAGTGGAGTGCCTGATTGGGGAGCACCTGCATGCAGGTATGAGCACCCTGTTCCTGGTGTACAGCAACAAGTGCCAGACCCCCCTGGGCATGGCCTCTGGACACATCAGAGACTTC |
| | CAGATCACTGCCAGTGGCCAGTATGGCCAGTGGGCCCCCAAGCTGGCCAGACTGCACTACTCAGGGAGCATCAATGCCTGGAGCACCAAGGAGCCCTTCAGCTGGATCAAGGTGGACCTGCTGGCCCCCATGATCATCCATGGTA |
| | TCAAGACCCAGGGTGCCAGACAGAAGTTCAGCAGCCTGTACATCAGCCAGTTCATCATCATGTACAGCCTGGATGGCAAGAAGTGGCAGACCTACAGAGGCAACAGCACAGGCACCCTGATGGTGTTCTTTGGCAATGTAGACAG |
| | CTCTGGCATCAAGCACAACATCTTCAACCCCCCCATCATAGCTAGATACATCAGACTGCACCCCACCCACTACAGCATCAGAAGCACCCTGAGAATGGAGCTGATGGGCTGTGACCTGAACAGCTGCAGCATGCCCCTGGGCATG |
| | GAGAGCAAGGCCATCTCAGATGCTCAGATCACTGCCAGCAGCTACTTCACCAACATGTTTGCCACCTGGAGCCCCAGCAAGGCCAGACTGCACCTGCAGGGCAGAAGCAATGCCTGGAGACCCCAGGTGAACAACCCCAAGGAGT |
| | GGCTGCAGGTGGACTTCCAGAAGACCATGAAGGTGACAGGAGTGACCACCCAGGGGGTGAAGAGCCTGCTGACCAGCATGTATGTGAAGGAGTTCCTGATCAGCAGCAGCCAGGATGGACACCAGTGGACCCTGTTCTTCCAGAA |
| | TGGAAAGGTGAAGGTGTTCCAGGGCAACCAGGACAGCTTCACCCCTGTGGTGAACAGCCTGGACCCCCCCCTGCTGACCAGATACCTGAGAATCCACCCCCAGAGCTGGGTGCACCAGATTGCCCTGAGAATGGAGGTGCTGGGC |
| | TGTGAGGCCCAGGACCTGTACTGA |
| 5 | FVIII-QQ05 |
| | ATGCAGATTGAGCTGAGCACCTGCTTCTTCCTGTGCCTGCTGAGATTCTGCTTCTCTGCTACCAGAAGATACTACCTGGGAGCTGTGGAGCTGAGCTGGGACTACATGCAGTCAGACCTGGGTGAACTGCCTGTGGATGCCAGAT |
| | TCCCCCCCAGAGTGCCCAAGAGCTTCCCCTTCAACACCAGTGTTGTCTACAAGAAGACCCTGTTTGTGGAGTTCACAGACCACCTGTTCAACATTGCCAAGCCCAGACCCCCCTGGATGGGCCTGCTGGGCCCCACCATCCAGGC |
| | TGAAGTGTATGATACAGTGGTGATCACCCTGAAGAACATGGCCAGCCACCCAGTGAGCCTGCATGCAGTGGGTGTGAGCTACTGGAAGGCCTCTGAAGGTGCAGAGTATGATGACCAGACCAGCCAGAGAGAGAAGGAGGATGAC |
| | AAGGTGTTCCCTGGGGGCAGCCACACCTATGTGTGGCAGGTGCTGAAGGAGAATGGCCCCATGGCCAGTGATCCCCTGTGCCTGACCTACAGCTACCTGAGCCATGTTGACCTGGTGAAGGACCTGAACTCAGGTCTGATTGGAG |
| | CCCTGCTGGTGTGCAGAGAGGGCAGCCTGGCCAAGGAGAAGACCCAGACCCTGCACAAGTTCATCCTGCTGTTTGCTGTCTTTGATGAAGGCAAGAGCTGGCACAGTGAGACCAAGAACAGCCTGATGCAGGACAGAGATGCAGC |
| | CTCTGCCAGAGCCTGGCCCAAGATGCACACAGTAAATGGCTATGTGAACAGAAGCCTGCCAGGGCTGATTGGCTGCCACAGAAAGTCTGTTTACTGGCATGTGATTGGGATGGGCACCACCCCTGAAGTGCACAGCATCTTCCTG |
| | GAGGGCCACACCTTCCTGGTGAGAAACCACAGACAGGCCAGCCTGGAGATCAGCCCCATCACCTTCCTGACAGCACAGACCCTGCTGATGGACCTGGGCCAGTTCCTGCTGTTCTGCCACATCAGCAGCCACCAGCATGATGGGA |
| | TGGAGGCCTATGTGAAGGTGGACAGCTGCCCAGAAGAGCCCCAGCTGCAGATGAAGAACAATGAGGAGGCAGAGGACTATGATGATGACCTGACTGACTCTGAGATGGATGTTGTGAGATTTGATGATGACAACAGCCCCAGCTT |
| | CATCCAGATCAGAAGTGTTGCCAAGAAGCACCCCAAGACCTGGGTGCACTACATTGCAGCTGAGGAGGAGGACTGGGACTATGCCCCCCTGGTGCTGGCCCCAGATGACAGAAGCTACAAGAGCCAGTACCTGAACAATGGCCCC |
| | CAGAGAATTGGTAGAAAGTACAAGAAGGTGAGATTCATGGCCTACACAGATGAGACCTTCAAGACCAGAGAGGCCATCCAGCATGAGTCAGGAATCCTGGGCCCCCTGCTGTATGGGGAGGTGGGGGATACCCTGCTGATCATCT |
| | TCAAGAACCAGGCCAGCAGACCCTACAACATCTACCCCCATGGCATCACAGATGTGAGACCCCTGTACAGCAGAAGACTGCCCAAGGGAGTGAAGCACCTGAAGGACTTCCCCATCCTGCCTGGTGAGATCTTCAAGTACAAGTG |
| | GACAGTTACTGTGGAAGATGGTCCCACCAAGTCAGACCCCAGATGCCTGACCAGATACTACAGCAGCTTTGTGAACATGGAGAGAGACCTGGCCTCTGGGCTGATTGGGCCCCTGCTGATCTGCTACAAGGAGAGTGTGGACCAG |
| | CAGGGCAACCAGATCATGTCTGATAAGAGAAATGTGATCCTGTTCTCTGTATTTGATGAAAACAGAAGCTGGTACCTGACTGAGAACATCCAGAGATTCCTGCCCAACCCAGCTGGAGTCCAGCTGGAGGACCCAGAGTTCCAGG |
| | CCAGCAACATCATGCACAGCATCAATGGATATGTGTTTGACAGCCTGCAGCTGTCTGTATGCCTGCATGAGGTGGCCTACTGGTACATCCTGAGCATTGGGGCCCAGACTGACTTCCTGTCTGTGTTCTTCTCAGGATACACCTT |
| | CAAGCACAAGATGGTGTATGAGGACACCCTGACCCTGTTCCCCTTCTCAGGGGAAACTGTGTTCATGAGCATGGAGAACCCTGGCCTGTGGATCCTGGGCTGCCACAACTCTGACTTCAGAAACAGAGGCATGACTGCCCTGCTG |
| | AAGGTGAGCAGCTGTGACAAGAACACTGGGGATTACTATGAGGACAGCTATGAGGACATCTCAGCCTACCTGCTGAGCAAGAACAATGCCATTGAACCCAGAAGCTTCAGCCAGAACCCCCCTGTCCTGAAGAGACACCAGAGAG |
| | AGATCACCAGAACCACCCTGCAGTCTGATCAGGAGGAGATTGACTATGATGATACCATCTCAGTGGAGATGAAGAAGGAGGACTTTGACATCTATGATGAAGATGAAAACCAGAGCCCCAGAAGCTTCCAGAAGAAGACCAGACA |
| | CTACTTCATTGCTGCTGTGGAGAGACTGTGGGACTATGGGATGAGCAGCAGCCCCCATGTGCTGAGAAACAGAGCCCAGTCAGGCAGTGTGCCCCAGTTCAAGAAGGTGGTGTTCCAGGAGTTCACAGATGGAAGCTTCACCCAG |
| | CCCCTGTACAGAGGTGAGCTGAATGAACACCTGGGCCTGCTGGGCCCCTACATCAGAGCTGAAGTGGAGGACAACATCATGGTGACCTTCAGAAACCAGGCCAGCAGACCCTACAGCTTCTACAGCAGCCTGATCAGCTATGAGG |
| | AGGACCAGAGACAGGGAGCAGAGCCCAGAAAGAACTTTGTCAAGCCCAATGAAACCAAGACCTACTTCTGGAAGGTGCAGCACCACATGGCCCCCACCAAGGATGAATTTGACTGCAAGGCCTGGGCCTACTTCTCTGATGTAGA |
| | CCTGGAGAAGGATGTGCACTCTGGGCTGATTGGCCCCCTGCTGGTGTGCCACACCAACACCCTGAACCCAGCCCATGGCAGACAGGTGACTGTGCAGGAGTTTGCCCTGTTCTTCACCATCTTTGATGAAACCAAGAGCTGGTAC |
| | TTCACTGAGAACATGGAGAGAAACTGCAGAGCCCCCTGCAACATCCAGATGGAGGACCCCACCTTCAAGGAGAACTACAGATTCCATGCTATCAATGGATACATCATGGACACCCTGCCTGGGCTGGTGATGGCCCAGGACCAGA |
| | GAATCAGATGGTACCTGCTGAGCATGGGCAGCAATGAGAACATCCACAGCATCCACTTCTCAGGACATGTCTTCACAGTAAGAAAGAAGGAGGAGTACAAGATGGCCCTGTACAACCTGTACCCAGGGGTCTTTGAAACTGTAGA |
| | GATGCTGCCCAGCAAGGCAGGCATCTGGAGAGTGGAGTGCCTGATTGGAGAGCACCTGCATGCTGGTATGAGCACCCTGTTCCTGGTGTACAGCAACAAGTGCCAGACCCCCCTGGGCATGGCCTCTGGCCACATCAGAGACTTC |
| | CAGATCACAGCCAGTGGCCAGTATGGGCAGTGGGCCCCCAAGCTGGCCAGACTGCACTACTCTGGCAGCATCAATGCCTGGAGCACCAAGGAGCCCTTCAGCTGGATCAAGGTGGACCTGCTGGCCCCCATGATCATCCATGGGA |
| | TCAAGACCCAGGGTGCCAGACAGAAGTTCAGCAGCCTGTACATCAGCCAGTTCATCATCATGTACAGCCTGGATGGCAAGAAGTGGCAGACCTACAGAGGCAACAGCACAGGTACCCTGATGGTGTTCTTTGGCAATGTGGACAG |
| | CTCAGGCATCAAGCACAACATCTTCAACCCCCCCATCATTGCCAGATACATCAGACTGCACCCCACCCACTACAGCATCAGAAGCACCCTGAGAATGGAGCTGATGGGCTGTGACCTGAACAGCTGCAGCATGCCCCTGGGCATG |
| | GAGAGCAAGGCCATCTCTGATGCCCAGATCACAGCTAGCAGCTACTTCACCAACATGTTTGCCACCTGGAGCCCCAGCAAGGCCAGACTGCACCTGCAGGGCAGAAGCAATGCTTGGAGACCCCAGGTGAACAACCCCAAGGAGT |
| | GGCTGCAGGTGGACTTCCAGAAGACCATGAAGGTGACAGGTGTGACCACCCAGGGGGTTAAGAGCCTGCTGACCAGCATGTATGTGAAGGAGTTCCTGATCAGCAGCAGCCAGGATGGCCACCAGTGGACCCTGTTCTTCCAGAA |
| | TGGCAAGGTGAAGGTGTTCCAGGGCAACCAGGACAGCTTCACCCCTGTTGTGAACAGCCTGGACCCCCCCCTGCTGACCAGATACCTGAGAATCCACCCCCAGAGCTGGGTGCACCAGATTGCCCTGAGAATGGAGGTGCTGGGC |
| | TGTGAGGCCCAGGACCTGTACTGA |
| 6 | FVIII-QQ06 |
| | ATGCAGATAGAACTGAGCACCTGCTTCTTCCTGTGCCTGCTGAGATTCTGCTTCTCAGCCACCAGAAGATACTACCTGGGGGCAGTTGAGCTGAGCTGGGACTACATGCAGAGTGATCTGGGGGAGCTGCCAGTGGATGCAAGAT |
| | TCCCCCCCAGAGTGCCCAAGAGCTTCCCCTTCAACACCAGTGTGGTCTACAAGAAGACCCTGTTTGTTGAGTTCACAGACCACCTGTTCAACATTGCTAAGCCCAGACCCCCCTGGATGGGCCTGCTGGGCCCCACCATCCAGGC |
| | AGAGGTGTATGATACAGTTGTGATCACCCTGAAGAACATGGCCAGCCACCCAGTTAGCCTGCATGCAGTTGGAGTGAGCTACTGGAAGGCCTCAGAGGGAGCTGAGTATGATGACCAGACCAGCCAGAGAGAGAAGGAGGATGAT |
| | AAGGTGTTCCCAGGGGGGAGCCACACCTATGTGTGGCAGGTGCTGAAGGAGAATGGACCCATGGCCAGTGACCCCCTGTGCCTGACCTACAGCTACCTGAGCCATGTGGACCTGGTGAAGGACCTGAACTCAGGACTGATAGGTG |
| | CTCTGCTGGTGTGCAGAGAGGGCAGCCTGGCCAAGGAGAAGACCCAGACCCTGCACAAGTTCATCCTGCTGTTTGCAGTTTTTGATGAAGGCAAGAGCTGGCACTCAGAGACCAAGAACAGCCTGATGCAGGACAGAGATGCAGC |
| | TTCTGCTAGAGCCTGGCCCAAGATGCACACTGTCAATGGCTATGTAAACAGAAGCCTGCCTGGCCTGATTGGCTGCCACAGAAAGTCTGTCTACTGGCATGTTATAGGTATGGGCACCACCCCTGAGGTGCACAGCATCTTCCTG |
| | GAGGGCCACACCTTCCTGGTGAGAAACCACAGACAGGCCAGCCTGGAGATCAGCCCCATCACCTTCCTGACTGCCCAGACCCTGCTGATGGACCTGGGCCAGTTCCTGCTGTTCTGCCACATCAGCAGCCACCAGCATGATGGCA |
| | TGGAGGCCTATGTCAAGGTGGACAGCTGCCCAGAAGAGCCCCAGCTGCAGATGAAGAACAATGAGGAGGCTGAGGACTATGATGATGACCTGACTGACAGTGAGATGGATGTAGTGAGATTTGATGATGACAACAGCCCCAGCTT |
| | CATCCAGATCAGAAGTGTAGCCAAGAAGCACCCCAAGACCTGGGTGCACTACATTGCTGCAGAGGAGGAGGACTGGGACTATGCCCCCCTGGTGCTGGCCCCAGATGACAGAAGCTACAAGAGCCAGTACCTGAACAATGGTCCC |
| | CAGAGAATTGGCAGAAAGTACAAGAAGGTGAGATTCATGGCCTACACAGATGAGACCTTCAAGACCAGAGAGGCCATCCAGCATGAGTCAGGCATCCTGGGCCCCCTGCTGTATGGAGAGGTGGGGGACACCCTGCTGATCATCT |
| | TCAAGAACCAGGCCAGCAGACCCTACAACATCTACCCCCATGGAATCACAGATGTGAGACCCCTGTACAGCAGAAGACTGCCCAAGGGGGTGAAGCACCTGAAGGACTTCCCCATCCTGCCTGGAGAGATCTTCAAGTACAAGTG |
| | GACTGTGACAGTAGAGGATGGACCCACCAAGTCAGACCCCAGATGCCTGACCAGATACTACAGCAGCTTTGTAAACATGGAGAGAGACCTGGCCAGTGGCCTGATAGGCCCCCTGCTGATCTGCTACAAGGAGTCAGTGGACCAG |
| | CAGGGCAACCAGATCATGTCAGACAAGAGAAATGTGATCCTGTTCTCTGTATTTGATGAGAACAGAAGCTGGTACCTGACTGAAAACATCCAGAGATTCCTGCCCAACCCTGCTGGAGTACAGCTGGAGGACCCTGAGTTCCAGG |
| | CCAGCAACATCATGCACAGCATCAATGGTTATGTGTTTGACAGCCTGCAGCTGTCTGTGTGCCTGCATGAAGTGGCCTACTGGTACATCCTGAGCATAGGTGCCCAGACTGACTTCCTGTCTGTGTTCTTCAGTGGCTACACCTT |
| | CAAGCACAAGATGGTGTATGAAGACACCCTGACCCTGTTCCCCTTCTCTGGGGAGACAGTATTCATGAGCATGGAGAACCCAGGACTGTGGATCCTGGGCTGCCACAACAGTGACTTCAGAAACAGAGGCATGACTGCTCTGCTG |
| | AAGGTGAGCAGCTGTGATAAGAACACAGGTGACTACTATGAGGACAGCTATGAAGACATCAGTGCTTACCTGCTGAGCAAGAACAATGCTATAGAACCCAGAAGCTTCAGCCAGAACCCCCCAGTTCTGAAGAGACACCAGAGAG |
| | AGATCACCAGAACCACCCTGCAGTCAGACCAGGAGGAGATAGACTATGATGATACCATCTCAGTAGAGATGAAGAAGGAGGACTTTGACATCTATGATGAGGATGAAAACCAGAGCCCCAGAAGCTTCCAGAAGAAGACCAGACA |
| | CTACTTCATAGCTGCTGTAGAGAGACTGTGGGACTATGGAATGAGCAGCAGCCCCCATGTGCTGAGAAACAGAGCCCAGTCTGGGAGTGTGCCCCAGTTCAAGAAGGTGGTGTTCCAGGAGTTCACAGATGGGAGCTTCACCCAG |
| | CCCCTGTACAGAGGGGAGCTGAATGAACACCTGGGCCTGCTGGGCCCCTACATCAGAGCAGAGGTGGAGGACAACATCATGGTGACCTTCAGAAACCAGGCCAGCAGACCCTACAGCTTCTACAGCAGCCTGATCAGCTATGAGG |
| | AGGACCAGAGACAGGGAGCTGAGCCCAGAAAGAACTTTGTTAAGCCCAATGAAACCAAGACCTACTTCTGGAAGGTGCAGCACCACATGGCCCCCACCAAGGATGAGTTTGACTGCAAGGCCTGGGCCTACTTCTCTGATGTTGA |
| | CCTGGAGAAGGATGTGCACTCTGGACTGATTGGGCCCCTGCTGGTGTGCCACACCAACACCCTGAACCCTGCCCATGGGAGACAGGTGACAGTACAGGAGTTTGCTCTGTTCTTCACCATCTTTGATGAGACCAAGAGCTGGTAC |
| | TTCACAGAAAACATGGAGAGAAACTGCAGAGCCCCCTGCAACATCCAGATGGAGGACCCCACCTTCAAGGAGAACTACAGATTCCATGCTATCAATGGCTACATCATGGACACCCTGCCTGGCCTGGTGATGGCCCAGGACCAGA |
| | GAATCAGATGGTACCTGCTGAGCATGGGCAGCAATGAAAACATCCACAGCATCCACTTCTCTGGTCATGTTTTCACAGTTAGAAAGAAGGAGGAGTACAAGATGGCCCTGTACAACCTGTACCCTGGGGTGTTTGAGACTGTGGA |
| | GATGCTGCCCAGCAAGGCTGGAATCTGGAGAGTGGAGTGCCTGATTGGAGAACACCTGCATGCAGGGATGAGCACCCTGTTCCTGGTGTACAGCAACAAGTGCCAGACCCCCCTGGGCATGGCCAGTGGTCACATCAGAGACTTC |
| | CAGATCACAGCCTCAGGCCAGTATGGACAGTGGGCCCCCAAGCTGGCCAGACTGCACTACAGTGGTAGCATCAATGCCTGGAGCACCAAGGAGCCCTTCAGCTGGATCAAGGTGGACCTGCTGGCCCCCATGATCATCCATGGAA |
| | TCAAGACCCAGGGGGCCAGACAGAAGTTCAGCAGCCTGTACATCAGCCAGTTCATCATCATGTACAGCCTGGATGGCAAGAAGTGGCAGACCTACAGAGGCAACAGCACTGGTACCCTGATGGTGTTCTTTGGCAATGTGGACAG |
| | CTCTGGGATCAAGCACAACATCTTCAACCCCCCCATCATAGCCAGATACATCAGACTGCACCCCACCCACTACAGCATCAGAAGCACCCTGAGAATGGAGCTGATGGGCTGTGACCTGAACAGCTGCAGCATGCCCCTGGGCATG |
| | GAGAGCAAGGCCATCAGTGATGCTCAGATCACAGCAAGCAGCTACTTCACCAACATGTTTGCCACCTGGAGCCCCAGCAAGGCCAGACTGCACCTGCAGGGCAGAAGCAATGCTTGGAGACCCCAGGTGAACAACCCCAAGGAGT |
| | GGCTGCAGGTGGACTTCCAGAAGACCATGAAGGTGACTGGGGTGACCACCCAGGGGGTGAAGAGCCTGCTGACCAGCATGTATGTCAAGGAGTTCCTGATCAGCAGCAGCCAGGATGGGCACCAGTGGACCCTGTTCTTCCAGAA |
| | TGGGAAGGTGAAGGTGTTCCAGGGCAACCAGGACAGCTTCACCCCAGTAGTGAACAGCCTGGACCCCCCCCTGCTGACCAGATACCTGAGAATCCACCCCCAGAGCTGGGTGCACCAGATAGCCCTGAGAATGGAGGTGCTGGGC |
| | TGTGAGGCCCAGGACCTGTACTGA |
| 7 | FVIII-0007 |
| | ATGCAGATAGAGCTGAGCACCTGCTTCTTCCTGTGCCTGCTGAGATTCTGCTTCTCAGCCACCAGAAGATACTACCTGGGTGCAGTGGAGCTGAGCTGGGACTACATGCAGAGTGACCTGGGGGAACTGCCTGTGGATGCCAGAT |
| | TCCCCCCCAGAGTGCCCAAGAGCTTCCCCTTCAACACCTCTGTTGTTTACAAGAAGACCCTGTTTGTAGAGTTCACTGACCACCTGTTCAACATAGCCAAGCCCAGACCCCCCTGGATGGGCCTGCTGGGCCCCACCATCCAGGC |
| | AGAGGTGTATGATACAGTGGTGATCACCCTGAAGAACATGGCCAGCCACCCTGTTAGCCTGCATGCAGTGGGGGTGAGCTACTGGAAGGCCAGTGAGGGAGCAGAGTATGATGATCAGACCAGCCAGAGAGAGAAGGAGGATGAC |
| | AAGGTGTTCCCTGGGGGCAGCCACACCTATGTATGGCAGGTGCTGAAGGAGAATGGCCCCATGGCCTCAGATCCCCTGTGCCTGACCTACAGCTACCTGAGCCATGTGGACCTGGTGAAGGACCTGAACAGTGGTCTGATTGGGG |
| | CCCTGCTGGTGTGCAGAGAGGGCAGCCTGGCCAAGGAGAAGACCCAGACCCTGCACAAGTTCATCCTGCTGTTTGCTGTCTTTGATGAGGGCAAGAGCTGGCACTCAGAGACCAAGAACAGCCTGATGCAGGACAGAGATGCTGC |
| | CTCTGCCAGAGCCTGGCCCAAGATGCACACTGTGAATGGGTATGTGAACAGAAGCCTGCCAGGTCTGATAGGCTGCCACAGAAAGTCAGTATACTGGCATGTGATTGGCATGGGCACCACCCCAGAGGTGCACAGCATCTTCCTG |
| | GAGGGCCACACCTTCCTGGTGAGAAACCACAGACAGGCCAGCCTGGAGATCAGCCCCATCACCTTCCTGACTGCCCAGACCCTGCTGATGGACCTGGGCCAGTTCCTGCTGTTCTGCCACATCAGCAGCCACCAGCATGATGGCA |
| | TGGAGGCCTATGTTAAGGTGGACAGCTGCCCAGAGGAGCCCCAGCTGCAGATGAAGAACAATGAAGAGGCTGAGGACTATGATGATGATCTGACAGACTCAGAAATGGATGTAGTCAGATTTGATGATGATAACAGCCCCAGCTT |
| | CATCCAGATCAGATCTGTAGCCAAGAAGCACCCCAAGACCTGGGTGCACTACATAGCAGCAGAGGAGGAGGACTGGGACTATGCACCCCTGGTGCTGGCCCCTGATGACAGAAGCTACAAGAGCCAGTACCTGAACAATGGCCCC |
| | CAGAGAATAGGCAGAAAGTACAAGAAGGTGAGATTCATGGCCTACACTGATGAGACCTTCAAGACCAGAGAGGCCATCCAGCATGAGTCTGGAATCCTGGGCCCCCTGCTGTATGGTGAGGTGGGTGACACCCTGCTGATCATCT |
| | TCAAGAACCAGGCCAGCAGACCCTACAACATCTACCCCCATGGCATCACTGATGTGAGACCCCTGTACAGCAGAAGACTGCCCAAGGGGGTGAAGCACCTGAAGGACTTCCCCATCCTGCCAGGTGAGATCTTCAAGTACAAGTG |
| | GACTGTGACAGTGGAGGATGGCCCCACCAAGAGTGACCCCAGATGCCTGACCAGATACTACAGCAGCTTTGTCAACATGGAGAGAGACCTGGCCAGTGGGCTGATAGGGCCCCTGCTGATCTGCTACAAGGAGTCAGTGGACCAG |
| | CAGGGCAACCAGATCATGAGTGATAAGAGAAATGTGATCCTGTTCTCAGTTTTTGATGAAAACAGAAGCTGGTACCTGACAGAAAACATCCAGAGATTCCTGCCCAACCCAGCAGGGGTACAGCTGGAGGACCCTGAATTCCAGG |
| | CCAGCAACATCATGCACAGCATCAATGGCTATGTATTTGACAGCCTGCAGCTGTCTGTTTGCCTGCATGAGGTGGCCTACTGGTACATCCTGAGCATAGGAGCCCAGACAGACTTCCTGAGTGTGTTCTTCAGTGGCTACACCTT |
| | CAAGCACAAGATGGTGTATGAGGACACCCTGACCCTGTTCCCCTTCTCAGGGGAGACTGTTTTCATGAGCATGGAGAACCCTGGACTGTGGATCCTGGGCTGCCACAACTCTGACTTCAGAAACAGAGGCATGACTGCCCTGCTG |
| | AAGGTGAGCAGCTGTGATAAGAACACTGGAGACTACTATGAGGACAGCTATGAGGACATCTCAGCCTACCTGCTGAGCAAGAACAATGCCATTGAACCCAGAAGCTTCAGCCAGAACCCCCCTGTACTGAAGAGACACCAGAGAG |
| | AGATCACCAGAACCACCCTGCAGAGTGACCAGGAGGAGATAGACTATGATGATACCATCTCTGTGGAGATGAAGAAGGAGGACTTTGATATCTATGATGAGGATGAGAACCAGAGCCCCAGAAGCTTCCAGAAGAAGACCAGACA |
| | CTACTTCATAGCAGCAGTGGAGAGACTGTGGGACTATGGCATGAGCAGCAGCCCCCATGTGCTGAGAAACAGAGCCCAGTCAGGCAGTGTTCCCCAGTTCAAGAAGGTGGTGTTCCAGGAGTTCACTGATGGTAGCTTCACCCAG |
| | CCCCTGTACAGAGGAGAGCTGAATGAGCACCTGGGCCTGCTGGGCCCCTACATCAGAGCTGAGGTGGAGGACAACATCATGGTGACCTTCAGAAACCAGGCCAGCAGACCCTACAGCTTCTACAGCAGCCTGATCAGCTATGAGG |
| | AGGACCAGAGACAGGGAGCAGAGCCCAGAAAGAACTTTGTTAAGCCCAATGAAACCAAGACCTACTTCTGGAAGGTGCAGCACCACATGGCCCCCACCAAGGATGAGTTTGATTGCAAGGCCTGGGCCTACTTCAGTGATGTAGA |
| | CCTGGAGAAGGATGTGCACTCAGGCCTGATTGGACCCCTGCTGGTGTGCCACACCAACACCCTGAACCCTGCACATGGAAGACAGGTGACAGTGCAGGAGTTTGCCCTGTTCTTCACCATCTTTGATGAGACCAAGAGCTGGTAC |
| | TTCACAGAGAACATGGAGAGAAACTGCAGAGCCCCCTGCAACATCCAGATGGAGGACCCCACCTTCAAGGAGAACTACAGATTCCATGCTATCAATGGCTACATCATGGACACCCTGCCTGGCCTGGTGATGGCCCAGGACCAGA |
| | GAATCAGATGGTACCTGCTGAGCATGGGCAGCAATGAGAACATCCACAGCATCCACTTCAGTGGGCATGTGTTCACAGTGAGAAAGAAGGAGGAGTACAAGATGGCCCTGTACAACCTGTACCCTGGAGTGTTTGAGACTGTGGA |
| | GATGCTGCCCAGCAAGGCAGGCATCTGGAGAGTGGAGTGCCTGATAGGAGAACACCTGCATGCTGGCATGAGCACCCTGTTCCTGGTGTACAGCAACAAGTGCCAGACCCCCCTGGGCATGGCCAGTGGACACATCAGAGACTTC |
| | CAGATCACAGCCTCTGGACAGTATGGACAGTGGGCCCCCAAGCTGGCCAGACTGCACTACTCAGGAAGCATCAATGCATGGAGCACCAAGGAGCCCTTCAGCTGGATCAAGGTGGACCTGCTGGCCCCCATGATCATCCATGGCA |
| | TCAAGACCCAGGGGGCAAGACAGAAGTTCAGCAGCCTGTACATCAGCCAGTTCATCATCATGTACAGCCTGGATGGCAAGAAGTGGCAGACCTACAGAGGCAACAGCACTGGAACCCTGATGGTGTTCTTTGGGAATGTGGACAG |
| | CTCAGGGATCAAGCACAACATCTTCAACCCCCCCATCATTGCTAGATACATCAGACTGCACCCCACCCACTACAGCATCAGAAGCACCCTGAGAATGGAGCTGATGGGCTGTGATCTGAACAGCTGCAGCATGCCCCTGGGCATG |
| | GAGAGCAAGGCCATCTCAGATGCCCAGATCACTGCCAGCAGCTACTTCACCAACATGTTTGCCACCTGGAGCCCCAGCAAGGCCAGACTGCACCTGCAGGGCAGAAGCAATGCTTGGAGACCCCAGGTGAACAACCCCAAGGAGT |
| | GGCTGCAGGTGGACTTCCAGAAGACCATGAAGGTGACTGGAGTAACCACCCAGGGGGTGAAGAGCCTGCTGACCAGCATGTATGTGAAGGAGTTCCTGATCAGCAGCAGCCAGGATGGTCACCAGTGGACCCTGTTCTTCCAGAA |
| | TGGCAAGGTGAAGGTGTTCCAGGGCAACCAGGACAGCTTCACCCCTGTTGTGAACAGCCTGGACCCCCCCCTGCTGACCAGATACCTGAGAATCCACCCCCAGAGCTGGGTGCACCAGATTGCCCTGAGAATGGAGGTGCTGGGC |
| | TGTGAGGCCCAGGACCTGTACTGA |
| 8 | FVIII-QQ08 |
| | ATGCAGATAGAGCTGAGCACCTGCTTCTTCCTGTGCCTGCTGAGATTCTGCTTCTCTGCCACCAGAAGATACTACCTGGGTGCAGTGGAGCTGAGCTGGGACTACATGCAGTCAGACCTGGGAGAGCTGCCAGTGGATGCTAGAT |
| | TCCCCCCCAGAGTGCCCAAGAGCTTCCCCTTCAACACCTCAGTGGTGTACAAGAAGACCCTGTTTGTAGAGTTCACTGACCACCTGTTCAACATTGCCAAGCCCAGACCCCCCTGGATGGGCCTGCTGGGCCCCACCATCCAGGC |
| | TGAGGTGTATGATACAGTGGTAATCACCCTGAAGAACATGGCCAGCCACCCAGTGAGCCTGCATGCTGTTGGAGTTAGCTACTGGAAGGCCTCTGAGGGTGCTGAGTATGATGATCAGACCAGCCAGAGAGAGAAGGAGGATGAT |
| | AAGGTGTTCCCAGGGGGAAGCCACACCTATGTATGGCAGGTGCTGAAGGAGAATGGGCCCATGGCCAGTGACCCCCTGTGCCTGACCTACAGCTACCTGAGCCATGTGGATCTGGTGAAGGACCTGAACTCAGGTCTGATAGGGG |
| | CCCTGCTGGTGTGCAGAGAGGGCAGCCTGGCCAAGGAGAAGACCCAGACCCTGCACAAGTTCATCCTGCTGTTTGCTGTATTTGATGAGGGCAAGAGCTGGCACAGTGAAACCAAGAACAGCCTGATGCAGGACAGAGATGCTGC |
| | CTCTGCCAGAGCCTGGCCCAAGATGCACACAGTGAATGGCTATGTGAACAGAAGCCTGCCTGGTCTGATAGGGTGCCACAGAAAGTCAGTGTACTGGCATGTCATTGGCATGGGCACCACCCCAGAGGTGCACAGCATCTTCCTG |
| | GAGGGCCACACCTTCCTGGTGAGAAACCACAGACAGGCCAGCCTGGAGATCAGCCCCATCACCTTCCTGACAGCTCAGACCCTGCTGATGGACCTGGGCCAGTTCCTGCTGTTCTGCCACATCAGCAGCCACCAGCATGATGGCA |
| | TGGAGGCCTATGTTAAGGTGGACAGCTGCCCAGAGGAGCCCCAGCTGCAGATGAAGAACAATGAGGAGGCAGAGGACTATGATGATGATCTGACAGACAGTGAGATGGATGTGGTGAGATTTGATGATGACAACAGCCCCAGCTT |
| | CATCCAGATCAGATCTGTGGCCAAGAAGCACCCCAAGACCTGGGTGCACTACATTGCAGCTGAAGAGGAGGACTGGGACTATGCCCCCCTGGTGCTGGCCCCAGATGATAGAAGCTACAAGAGCCAGTACCTGAACAATGGCCCC |
| | CAGAGAATTGGAAGAAAGTACAAGAAGGTGAGATTCATGGCCTACACTGATGAAACCTTCAAGACCAGAGAGGCCATCCAGCATGAGTCAGGCATCCTGGGCCCCCTGCTGTATGGTGAGGTGGGTGATACCCTGCTGATCATCT |
| | TCAAGAACCAGGCCAGCAGACCCTACAACATCTACCCCCATGGCATCACTGATGTAAGACCCCTGTACAGCAGAAGACTGCCCAAGGGTGTAAAGCACCTGAAGGACTTCCCCATCCTGCCTGGGGAAATCTTCAAGTACAAGTG |
| | GACTGTGACTGTAGAGGATGGTCCCACCAAGAGTGACCCCAGATGCCTGACCAGATACTACAGCAGCTTTGTGAACATGGAGAGAGACCTGGCCTCTGGTCTGATAGGTCCCCTGCTGATCTGCTACAAGGAGTCAGTAGACCAG |
| | CAGGGCAACCAGATCATGTCTGACAAGAGAAATGTGATCCTGTTCAGTGTGTTTGATGAGAACAGAAGCTGGTACCTGACAGAAAACATCCAGAGATTCCTGCCCAACCCAGCTGGAGTGCAGCTGGAGGACCCTGAGTTCCAGG |
| | CCAGCAACATCATGCACAGCATCAATGGGTATGTCTTTGACAGCCTGCAGCTGAGTGTGTGCCTGCATGAGGTGGCCTACTGGTACATCCTGAGCATTGGTGCTCAGACAGATTTCCTGTCTGTGTTCTTCAGTGGTTACACCTT |
| | CAAGCACAAGATGGTGTATGAAGACACCCTGACCCTGTTCCCCTTCAGTGGAGAGACAGTATTCATGAGCATGGAGAACCCTGGCCTGTGGATCCTGGGCTGCCACAACAGTGATTTCAGAAACAGAGGCATGACTGCACTGCTG |
| | AAGGTGAGCAGCTGTGACAAGAACACTGGGGATTACTATGAAGACAGCTATGAGGACATCAGTGCCTACCTGCTGAGCAAGAACAATGCTATTGAGCCCAGAAGCTTCAGCCAGAACCCCCCAGTCCTGAAGAGACACCAGAGAG |
| | AGATCACCAGAACCACCCTGCAGAGTGACCAGGAGGAGATAGACTATGATGACACCATCTCAGTGGAGATGAAGAAGGAGGACTTTGACATCTATGATGAGGATGAGAACCAGAGCCCCAGAAGCTTCCAGAAGAAGACCAGACA |
| | CTACTTCATTGCAGCAGTAGAGAGACTGTGGGACTATGGGATGAGCAGCAGCCCCCATGTACTGAGAAACAGAGCCCAGTCTGGATCTGTGCCCCAGTTCAAGAAGGTGGTGTTCCAGGAGTTCACTGATGGCAGCTTCACCCAG |
| | CCCCTGTACAGAGGAGAGCTGAATGAGCACCTGGGCCTGCTGGGCCCCTACATCAGAGCAGAGGTGGAGGACAACATCATGGTGACCTTCAGAAACCAGGCCAGCAGACCCTACAGCTTCTACAGCAGCCTGATCAGCTATGAGG |
| | AGGACCAGAGACAGGGTGCTGAGCCCAGAAAGAACTTTGTTAAGCCCAATGAGACCAAGACCTACTTCTGGAAGGTGCAGCACCACATGGCCCCCACCAAGGATGAATTTGATTGCAAGGCCTGGGCCTACTTCAGTGATGTAGA |
| | CCTGGAGAAGGATGTGCACAGTGGACTGATTGGGCCCCTGCTGGTGTGCCACACCAACACCCTGAACCCAGCCCATGGCAGACAGGTGACTGTGCAGGAGTTTGCTCTGTTCTTCACCATCTTTGATGAAACCAAGAGCTGGTAC |
| | TTCACAGAGAACATGGAGAGAAACTGCAGAGCCCCCTGCAACATCCAGATGGAGGACCCCACCTTCAAGGAGAACTACAGATTCCATGCTATCAATGGCTACATCATGGACACCCTGCCAGGTCTGGTGATGGCCCAGGACCAGA |
| | GAATCAGATGGTACCTGCTGAGCATGGGCAGCAATGAGAACATCCACAGCATCCACTTCTCTGGCCATGTCTTCACTGTGAGAAAGAAGGAGGAGTACAAGATGGCCCTGTACAACCTGTACCCAGGTGTCTTTGAGACTGTAGA |
| | GATGCTGCCCAGCAAGGCTGGCATCTGGAGAGTGGAGTGCCTGATTGGAGAGCACCTGCATGCAGGAATGAGCACCCTGTTCCTGGTGTACAGCAACAAGTGCCAGACCCCCCTGGGCATGGCCAGTGGCCACATCAGAGACTTC |
| | CAGATCACAGCCTCTGGGCAGTATGGACAGTGGGCCCCCAAGCTGGCCAGACTGCACTACTCAGGAAGCATCAATGCCTGGAGCACCAAGGAGCCCTTCAGCTGGATCAAGGTGGACCTGCTGGCCCCCATGATCATCCATGGGA |
| | TCAAGACCCAGGGGGCCAGACAGAAGTTCAGCAGCCTGTACATCAGCCAGTTCATCATCATGTACAGCCTGGATGGTAAGAAGTGGCAGACCTACAGAGGCAACAGCACAGGCACCCTGATGGTGTTCTTTGGAAATGTAGACAG |
| | CTCTGGCATCAAGCACAACATCTTCAACCCCCCCATCATAGCCAGATACATCAGACTGCACCCCACCCACTACAGCATCAGAAGCACCCTGAGAATGGAGCTGATGGGCTGTGACCTGAACAGCTGCAGCATGCCCCTGGGCATG |
| | GAGAGCAAGGCCATCTCTGATGCTCAGATCACTGCCAGCAGCTACTTCACCAACATGTTTGCAACCTGGAGCCCCAGCAAGGCCAGACTGCACCTGCAGGGCAGAAGCAATGCCTGGAGACCCCAGGTGAACAACCCCAAGGAGT |
| | GGCTGCAGGTGGACTTCCAGAAGACCATGAAGGTGACTGGTGTAACCACCCAGGGAGTGAAGAGCCTGCTGACCAGCATGTATGTCAAGGAGTTCCTGATCAGCAGCAGCCAGGATGGTCACCAGTGGACCCTGTTCTTCCAGAA |
| | TGGAAAGGTGAAGGTGTTCCAGGGCAACCAGGACAGCTTCACCCCAGTTGTGAACAGCCTGGACCCCCCCCTGCTGACCAGATACCTGAGAATCCACCCCCAGAGCTGGGTGCACCAGATTGCCCTGAGAATGGAGGTGCTGGGC |
| | TGTGAGGCCCAGGACCTGTACTGA |
| 9 | FVIII-QQ09 |
| | ATGCAGATAGAGCTGAGCACCTGCTTCTTCCTGTGCCTGCTGAGATTCTGCTTCAGTGCAACCAGAAGATACTACCTGGGTGCAGTGGAGCTGAGCTGGGACTACATGCAGTCTGATCTGGGGGAGCTGCCAGTTGATGCCAGAT |
| | TCCCCCCCAGAGTGCCCAAGAGCTTCCCCTTCAACACCAGTGTTGTTTACAAGAAGACCCTGTTTGTGGAATTCACTGACCACCTGTTCAACATAGCCAAGCCCAGACCCCCCTGC TGGGCCTGCTGGGCCCCACCATCCAGGC |
| | AGAGGTGTATGACACAGTAGTGATCACCCTGAAGAACATGGCCAGCCACCCTGTGAGCCTGCATGCAGTAGGGGTAAGCTACTGGAAGGCCTCTGAAGGTGCTGAATATGATGACCAGACCAGCCAGAGAGAGAAGGAGGATGAC |
| | AAGGTGTTCCCAGGTGGAAGCCACACCTATGTCTGGCAGGTGCTGAAGGAGAATGGCCCCATGGCCAGTGACCCCCTGTGCCTGACCTACAGCTACCTGAGCCATGTGGACCTGGTGAAGGACCTGAACTCAGGTCTGATTGGAG |
| | CACTGCTGGTGTGCAGAGAGGGCAGCCTGGCCAAGGAGAAGACCCAGACCCTGCACAAGTTCATCCTGCTGTTTGCAGTGTTTGATGAAGGCAAGAGCTGGCACAGTGAGACCAAGAACAGCCTGATGCAGGACAGAGATGCAGC |
| | CAGTGCCAGAGCCTGGCCCAAGATGCACACTGTGAATGGTTATGTCAACAGAAGCCTGCCAGGGCTGATAGGCTGCCACAGAAAGAGTGTTTACTGGCATGTAATTGGGATGGGCACCACCCCAGAGGTGCACAGCATCTTCCTG |
| | GAGGGCCACACCTTCCTGGTGAGAAACCACAGACAGGCCAGCCTGGAGATCAGCCCCATCACCTTCCTGACAGCTCAGACCCTGCTGATGGACCTGGGCCAGTTCCTGCTGTTCTGCCACATCAGCAGCCACCAGCATGATGGCA |
| | TGGAGGCCTATGTAAAGGTGGACAGCTGCCCTGAGGAGCCCCAGCTGCAGATGAAGAACAATGAGGAGGCAGAGGACTATGATGATGACCTGACTGACAGTGAGATGGATGTGGTGAGATTTGATGATGACAACAGCCCCAGCTT |
| | CATCCAGATCAGAAGTGTTGCTAAGAAGCACCCCAAGACCTGGGTGCACTACATTGCAGCAGAGGAGGAGGACTGGGACTATGCTCCCCTGGTGCTGGCCCCTGATGACAGAAGCTACAAGAGCCAGTACCTGAACAATGGCCCC |
| | CAGAGAATTGGCAGAAAGTACAAGAAGGTGAGATTCATGGCCTACACTGATGAGACCTTCAAGACCAGAGAGGCCATCCAGCATGAAAGTGGCATCCTGGGCCCCCTGCTGTATGGAGAGGTGGGAGACACCCTGCTGATCATCT |
| | TCAAGAACCAGGCCAGCAGACCCTACAACATCTACCCCCATGGTATCACAGATGTGAGACCCCTGTACAGCAGAAGACTGCCCAAGGGGGTGAAGCACCTGAAGGACTTCCCCATCCTGCCAGGTGAAATCTTCAAGTACAAGTG |
| | GACAGTCACAGTGGAGGATGGCCCCACCAAGAGTGACCCCAGATGCCTGACCAGATACTACAGCAGCTTTGTGAACATGGAGAGAGACCTGGCCTCTGGCCTGATAGGCCCCCTGCTGATCTGCTACAAGGAGTCAGTGGACCAG |
| | CAGGGCAACCAGATCATGAGTGATAAGAGAAATGTGATCCTGTTCTCAGTATTTGATGAGAACAGAAGCTGGTACCTGACTGAAAACATCCAGAGATTCCTGCCCAACCCTGCAGGGGTGCAGCTGGAGGACCCTGAATTCCAGG |
| | CCAGCAACATCATGCACAGCATCAATGGCTATGTATTTGATAGCCTGCAGCTGAGTGTGTGCCTGCATGAGGTGGCCTACTGGTACATCCTGAGCATAGGGGCCCAGACAGACTTCCTGTCTGTGTTCTTCTCAGGTTACACCTT |
| | CAAGCACAAGATGGTGTATGAAGACACCCTGACCCTGTTCCCCTTCAGTGGGGAAACAGTATTCATGAGCATGGAGAACCCTGGGCTGTGGATCCTGGGCTGCCACAACTCAGATTTCAGAAACAGAGGCATGACTGCCCTGCTG |
| | AAGGTGAGCAGCTGTGACAAGAACACTGGAGACTACTATGAGGACAGCTATGAGGACATCAGTGCCTACCTGCTGAGCAAGAACAATGCTATAGAGCCCAGAAGCTTCAGCCAGAACCCCCCAGTCCTGAAGAGACACCAGAGAG |
| | AGATCACCAGAACCACCCTGCAGTCAGATCAGGAGGAGATAGACTATGATGACACCATCAGTGTGGAGATGAAGAAGGAGGACTTTGACATCTATGATGAGGATGAGAACCAGAGCCCCAGAAGCTTCCAGAAGAAGACCAGACA |
| | CTACTTCATAGCTGCTGTAGAGAGACTGTGGGACTATGGCATGAGCAGCAGCCCCCATGTCCTGAGAAACAGAGCCCAGTCTGGCTCTGTTCCCCAGTTCAAGAAGGTGGTGTTCCAGGAGTTCACAGATGGCAGCTTCACCCAG |
| | CCCCTGTACAGAGGGGAGCTGAATGAACACCTGGGCCTGCTGGGCCCCTACATCAGAGCTGAAGTGGAGGACAACATCATGGTGACCTTCAGAAACCAGGCCAGCAGACCCTACAGCTTCTACAGCAGCCTGATCAGCTATGAGG |
| | AGGACCAGAGACAGGGTGCAGAACCCAGAAAGAACTTTGTTAAGCCCAATGAGACCAAGACCTACTTCTGGAAGGTGCAGCACCACATGGCCCCCACCAAGGATGAATTTGACTGCAAGGCCTGGGCCTACTTCTCAGATGTTGA |
| | CCTGGAGAAGGATGTCCACTCAGGACTGATAGGGCCCCTGCTGGTGTGCCACACCAACACCCTGAACCCAGCCCATGGCAGACAGGTGACTGTGCAGGAGTTTGCTCTGTTCTTCACCATCTTTGATGAGACCAAGAGCTGGTAC |
| | TTCACAGAGAACATGGAGAGAAACTGCAGAGCCCCCTGCAACATCCAGATGGAGGACCCCACCTTCAAGGAGAACTACAGATTCCATGCTATCAATGGATACATCATGGACACCCTGCCAGGCCTGGTGATGGCCCAGGACCAGA |
| | GAATCAGATGGTACCTGCTGAGCATGGGCAGCAATGAGAACATCCACAGCATCCACTTCAGTGGTCATGTGTTCACAGTAAGAAAGAAGGAGGAGTACAAGATGGCCCTGTACAACCTGTACCCAGGGGTGTTTGAAACTGTGGA |
| | AATGCTGCCCAGCAAGGCAGGGATCTGGAGAGTGGAGTGCCTGATTGGAGAACACCTGCATGCAGGCATGAGCACCCTGTTCCTGGTGTACAGCAACAAGTGCCAGACCCCCCTGGGCATGGCCAGTGGGCACATCAGAGACTTC |
| | CAGATCACAGCCTCTGGGCAGTATGGCCAGTGGGCCCCCAAGCTGGCCAGACTGCACTACTCAGGCAGCATCAATGCCTGGAGCACCAAGGAGCCCTTCAGCTGGATCAAGGTGGACCTGCTGGCCCCCATGATCATCCATGGCA |
| | TCAAGACCCAGGGAGCAAGACAGAAGTTCAGCAGCCTGTACATCAGCCAGTTCATCATCATGTACAGCCTGGATGGGAAGAAGTGGCAGACCTACAGAGGCAACAGCACTGGGACCCTGATGGTGTTCTTTGGCAATGTAGACAG |
| | CTCTGGCATCAAGCACAACATCTTCAACCCCCCCATCATTGCAAGATACATCAGACTGCACCCCACCCACTACAGCATCAGAAGCACCCTGAGAATGGAGCTGATGGGCTGTGACCTGAACAGCTGCAGCATGCCCCTGGGCATG |
| | GAGAGCAAGGCCATCAGTGATGCCCAGATCACTGCTAGCAGCTACTTCACCAACATGTTTGCCACCTGGAGCCCCAGCAAGGCCAGACTGCACCTGCAGGGCAGAAGCAATGCATGGAGACCCCAGGTGAACAACCCCAAGGAGT |
| | GGCTGCAGGTGGACTTCCAGAAGACCATGAAGGTGACAGGGGTTACCACCCAGGGTGTGAAGAGCCTGCTGACCAGCATGTATGTGAAGGAGTTCCTGATCAGCAGCAGCCAGGATGGCCACCAGTGGACCCTGTTCTTCCAGAA |
| | TGGGAAGGTGAAGGTGTTCCAGGGCAACCAGGACAGCTTCACCCCTGTGGTGAACAGCCTGGACCCCCCCCTGCTGACCAGATACCTGAGAATCCACCCCCAGAGCTGGGTGCACCAGATTGCCCTGAGAATGGAGGTGCTGGGC |
| | TGTGAGGCCCAGGACCTGTACTGA |
| 10 | FVIII-QQ10 |
| | ATGCAGATAGAACTGAGCACCTGCTTCTTCCTGTGCCTGCTGAGATTCTGCTTCTCTGCCACCAGAAGATACTACCTGGGGGCTGTGGAGCTGAGCTGGGACTACATGCAGAGTGACCTGGGAGAGCTGCCAGTTGATGCAAGAT |
| | TCCCCCCCAGAGTGCCCAAGAGCTTCCCCTTCAACACCTCTGTGGTGTACAAGAAGACCCTGTTTGTGGAGTTCACTGATCACCTGTTCAACATAGCCAAGCCCAGACCCCCCTGGATGGGCCTGCTGGGCCCCACCATCCAGGC |
| | TGAAGTGTATGACACTGTAGTGATCACCCTGAAGAACATGGCCAGCCACCCTGTGAGCCTGCATGCTGTGGGAGTGAGCTACTGGAAGGCCTCAGAGGGAGCAGAGTATGATGACCAGACCAGCCAGAGAGAGAAGGAGGATGAC |
| | AAGGTGTTCCCAGGAGGCAGCCACACCTATGTTTGGCAGGTGCTGAAGGAGAATGGGCCCATGGCCTCAGACCCCCTGTGCCTGACCTACAGCTACCTGAGCCATGTAGACCTGGTGAAGGACCTGAACTCTGGCCTGATAGGAG |
| | CCCTGCTGGTGTGCAGAGAGGGCAGCCTGGCCAAGGAGAAGACCCAGACCCTGCACAAGTTCATCCTGCTGTTTGCAGTGTTTGATGAGGGCAAGAGCTGGCACTCAGAGACCAAGAACAGCCTGATGCAGGACAGAGATGCAGC |
| | CAGTGCTAGAGCCTGGCCCAAGATGCACACAGTGAATGGGTATGTTAACAGAAGCCTGCCAGGCCTGATAGGATGCCACAGAAAGTCTGTGTACTGGCATGTGATTGGTATGGGCACCACCCCAGAGGTGCACAGCATCTTCCTG |
| | GAGGGCCACACCTTCCTGGTGAGAAACCACAGACAGGCCAGCCTGGAGATCAGCCCCATCACCTTCCTGACAGCCCAGACCCTGCTGATGGACCTGGGCCAGTTCCTGCTGTTCTGCCACATCAGCAGCCACCAGCATGATGGCA |
| | TGGAGGCCTATGTTAAGGTGGACAGCTGCCCAGAGGAGCCCCAGCTGCAGATGAAGAACAATGAAGAGGCAGAGGACTATGATGATGACCTGACAGATTCAGAGATGGATGTGGTGAGATTTGATGATGACAACAGCCCCAGCTT |
| | CATCCAGATCAGAAGTGTGGCCAAGAAGCACCCCAAGACCTGGGTGCACTACATTGCTGCTGAAGAGGAGGACTGGGACTATGCCCCCCTGGTGCTGGCCCCAGATGACAGAAGCTACAAGAGCCAGTACCTGAACAATGGGCCC |
| | CAGAGAATTGGAAGAAAGTACAAGAAGGTGAGATTCATGGCCTACACAGATGAAACCTTCAAGACCAGAGAGGCCATCCAGCATGAGAGTGGGATCCTGGGCCCCCTGCTGTATGGGGAGGTGGGTGATACCCTGCTGATCATCT |
| | TCAAGAACCAGGCCAGCAGACCCTACAACATCTACCCCCATGGCATCACAGATGTGAGACCCCTGTACAGCAGAAGACTGCCCAAGGGAGTTAAGCACCTGAAGGACTTCCCCATCCTGCCAGGAGAGATCTTCAAGTACAAGTG |
| | GACAGTCACAGTGGAGGATGGGCCCACCAAGTCTGACCCCAGATGCCTGACCAGATACTACAGCAGCTTTGTAAACATGGAGAGAGACCTGGCCTCAGGTCTGATAGGCCCCCTGCTGATCTGCTACAAGGAGAGTGTTGATCAG |
| | CAGGGCAACCAGATCATGTCTGACAAGAGAAATGTGATCCTGTTCTCAGTGTTTGATGAAAACAGAAGCTGGTACCTGACTGAGAACATCCAGAGATTCCTGCCCAACCCAGCTGGGGTGCAGCTGGAGGACCCTGAGTTCCAGG |
| | CCAGCAACATCATGCACAGCATCAATGGATATGTGTTTGACAGCCTGCAGCTGTCAGTGTGCCTGCATGAGGTGGCCTACTGGTACATCCTGAGCATAGGAGCTCAGACAGACTTCCTGAGTGTATTCTTCTCAGGGTACACCTT |
| | CAAGCACAAGATGGTGTATGAGGACACCCTGACCCTGTTCCCCTTCTCAGGTGAGACAGTGTTCATGAGCATGGAGAACCCAGGGCTGTGGATCCTGGGCTGCCACAACTCTGATTTCAGAAACAGAGGCATGACTGCCCTGCTG |
| | AAGGTGAGCAGCTGTGACAAGAACACAGGGGACTACTATGAGGACAGCTATGAGGACATCTCTGCCTACCTGCTGAGCAAGAACAATGCTATTGAGCCCAGAAGCTTCAGCCAGAACCCCCCTGTACTGAAGAGACACCAGAGAG |
| | AGATCACCAGAACCACCCTGCAGAGTGATCAGGAGGAGATAGATTATGATGACACCATCTCAGTGGAGATGAAGAAGGAGGACTTTGATATCTATGATGAGGATGAAAACCAGAGCCCCAGAAGCTTCCAGAAGAAGACCAGACA |
| | CTACTTCATTGCAGCTGTAGAGAGACTGTGGGACTATGGTATGAGCAGCAGCCCCCATGTCCTGAGAAACAGAGCCCAGTCTGGATCAGTCCCCCAGTTCAAGAAGGTGGTGTTCCAGGAGTTCACAGATGGTAGCTTCACCCAG |
| | CCCCTGTACAGAGGAGAGCTGAATGAGCACCTGGGCCTGCTGGGCCCCTACATCAGAGCAGAGGTGGAGGACAACATCATGGTGACCTTCAGAAACCAGGCCAGCAGACCCTACAGCTTCTACAGCAGCCTGATCAGCTATGAAG |
| | AGGACCAGAGACAGGGAGCAGAGCCCAGAAAGAACTTTGTGAAGCCCAATGAGACCAAGACCTACTTCTGGAAGGTGCAGCACCACATGGCCCCCACCAAGGATGAGTTTGATTGCAAGGCCTGGGCCTACTTCAGTGATGTTGA |
| | CCTGGAGAAGGATGTTCACAGTGGCCTGATAGGTCCCCTGCTGGTGTGCCACACCAACACCCTGAACCCAGCCCATGGCAGACAGGTGACTGTCCAGGAGTTTGCCCTGTTCTTCACCATCTTTGATGAGACCAAGAGCTGGTAC |
| | TTCACTGAAAACATGGAGAGAAACTGCAGAGCCCCCTGCAACATCCAGATGGAGGACCCCACCTTCAAGGAGAACTACAGATTCCATGCCATCAATGGCTACATCATGGACACCCTGCCTGGACTGGTGATGGCCCAGGACCAGA |
| | GAATCAGATGGTACCTGCTGAGCATGGGCAGCAATGAGAACATCCACAGCATCCACTTCTCAGGCCATGTATTCACTGTGAGAAAGAAGGAGGAGTACAAGATGGCCCTGTACAACCTGTACCCAGGTGTGTTTGAGACAGTGGA |
| | GATGCTGCCCAGCAAGGCAGGTATCTGGAGAGTGGAGTGCCTGATAGGAGAACACCTGCATGCTGGCATGAGCACCCTGTTCCTGGTGTACAGCAACAAGTGCCAGACCCCCCTGGGCATGGCCAGTGGCCACATCAGAGACTTC |
| | CAGATCACAGCCTCAGGCCAGTATGGGCAGTGGGCCCCCAAGCTGGCCAGACTGCACTACTCTGGCAGCATCAATGCATGGAGCACCAAGGAGCCCTTCAGCTGGATCAAGGTGGACCTGCTGGCCCCCATGATCATCCATGGAA |
| | TCAAGACCCAGGGTGCTAGACAGAAGTTCAGCAGCCTGTACATCAGCCAGTTCATCATCATGTACAGCCTGGATGGCAAGAAGTGGCAGACCTACAGAGGCAACAGCACTGGCACCCTGATGGTGTTCTTTGGCAATGTTGACAG |
| | CTCTGGCATCAAGCACAACATCTTCAACCCCCCCATCATAGCAAGATACATCAGACTGCACCCCACCCACTACAGCATCAGAAGCACCCTGAGAATGGAGCTGATGGGCTGTGATCTGAACAGCTGCAGCATGCCCCTGGGCATG |
| | GAGAGCAAGGCCATCAGTGATGCTCAGATCACTGCCAGCAGCTACTTCACCAACATGTTTGCAACCTGGAGCCCCAGCAAGGCCAGACTGCACCTGCAGGGCAGAAGCAATGCCTGGAGACCCCAGGTGAACAACCCCAAGGAGT |
| | GGCTGCAGGTGGACTTCCAGAAGACCATGAAGGTGACAGGTGTTACCACCCAGGGTGTCAAGAGCCTGCTGACCAGCATGTATGTAAAGGAGTTCCTGATCAGCAGCAGCCAGGATGGCCACCAGTGGACCCTGTTCTTCCAGAA |
| | TGGAAAGGTGAAGGTGTTCCAGGGCAACCAGGACAGCTTCACCCCTGTGGTGAACAGCCTGGACCCCCCCCTGCTGACCAGATACCTGAGAATCCACCCCCAGAGCTGGGTGCACCAGATAGCCCTGAGAATGGAGGTGCTGGGC |
| | TGTGAAGCCCAGGACCTGTACTGA |
| 11 | FVIII-QQ11 |
| | ATGCAGATAGAGCTGAGCACCTGCTTCTTCCTGTGCCTGCTGAGATTCTGCTTCTCTGCCACCAGAAGATACTACCTGGGTGCTGTTGAGCTGAGCTGGGACTACATGCAGTCTGATCTGGGTGAGCTGCCTGTTGATGCCAGAT |
| | TCCCCCCCAGAGTGCCCAAGAGCTTCCCCTTCAACACCTCAGTTGTGTACAAGAAGACCCTGTTTGTTGAGTTCACAGACCACCTGTTCAACATTGCTAAGCCCAGACCCCCCTGGATGGGCCTGCTGGGCCCCACCATCCAGGC |
| | AGAGGTGTATGATACAGTTGTGATCACCCTGAAGAACATGGCCAGCCACCCAGTGAGCCTGCATGCAGTAGGTGTGAGCTACTGGAAGGCCAGTGAGGGGGCTGAATATGATGATCAGACCAGCCAGAGAGAGAAGGAGGATGAC |
| | AAGGTGTTCCCAGGTGGTAGCCACACCTATGTATGGCAGGTGCTGAAGGAGAATGGCCCCATGGCCAGTGACCCCCTGTGCCTGACCTACAGCTACCTGAGCCATGTGGACCTGGTGAAGGACCTGAACTCTGGACTGATTGGAG |
| | CACTGCTGGTGTGCAGAGAGGGCAGCCTGGCCAAGGAGAAGACCCAGACCCTGCACAAGTTCATCCTGCTGTTTGCAGTGTTTGATGAGGGCAAGAGCTGGCACTCAGAGACCAAGAACAGCCTGATGCAGGACAGAGATGCAGC |
| | TTCTGCCAGAGCCTGGCCCAAGATGCACACTGTCAATGGCTATGTGAACAGAAGCCTGCCAGGCCTGATTGGTTGCCACAGAAAGAGTGTGTACTGGCATGTTATAGGGATGGGCACCACCCCTGAGGTGCACAGCATCTTCCTG |
| | GAGGGCCACACCTTCCTGGTGAGAAACCACAGACAGGCCAGCCTGGAGATCAGCCCCATCACCTTCCTGACAGCACAGACCCTGCTGATGGACCTGGGCCAGTTCCTGCTGTTCTGCCACATCAGCAGCCACCAGCATGATGGCA |
| | TGGAGGCCTATGTTAAGGTGGACAGCTGCCCTGAGGAGCCCCAGCTGCAGATGAAGAACAATGAGGAGGCTGAGGACTATGATGATGACCTGACAGACTCAGAGATGGATGTGGTGAGATTTGATGATGATAACAGCCCCAGCTT |
| | CATCCAGATCAGATCAGTGGCCAAGAAGCACCCCAAGACCTGGGTGCACTACATAGCTGCTGAAGAGGAGGACTGGGACTATGCCCCCCTGGTGCTGGCCCCAGATGACAGAAGCTACAAGAGCCAGTACCTGAACAATGGGCCC |
| | CAGAGAATTGGCAGAAAGTACAAGAAGGTGAGATTCATGGCCTACACTGATGAGACCTTCAAGACCAGAGAGGCCATCCAGCATGAGTCTGGAATCCTGGGCCCCCTGCTGTATGGAGAGGTGGGTGACACCCTGCTGATCATCT |
| | TCAAGAACCAGGCCAGCAGACCCTACAACATCTACCCCCATGGCATCACTGATGTTAGACCCCTGTACAGCAGAAGACTGCCCAAGGGTGTGAAGCACCTGAAGGACTTCCCCATCCTGCCTGGGGAGATCTTCAAGTACAAGTG |
| | GACAGTCACTGTAGAGGATGGCCCCACCAAGAGTGACCCCAGATGCCTGACCAGATACTACAGCAGCTTTGTGAACATGGAGAGAGACCTGGCCTCTGGACTGATTGGACCCCTGCTGATCTGCTACAAGGAGAGTGTGGACCAG |
| | CAGGGCAACCAGATCATGTCAGATAAGAGAAATGTCATCCTGTTCTCAGTATTTGATGAGAACAGAAGCTGGTACCTGACTGAGAACATCCAGAGATTCCTGCCCAACCCAGCTGGGGTGCAGCTGGAGGACCCTGAATTCCAGG |
| | CCAGCAACATCATGCACAGCATCAATGGCTATGTATTTGATAGCCTGCAGCTGTCTGTGTGCCTGCATGAGGTGGCCTACTGGTACATCCTGAGCATTGGGGCCCAGACTGATTTCCTGTCTGTCTTCTTCTCTGGGTACACCTT |
| | CAAGCACAAGATGGTGTATGAGGACACCCTGACCCTGTTCCCCTTCTCAGGAGAGACAGTGTTCATGAGCATGGAGAACCCAGGCCTGTGGATCCTGGGCTGCCACAACTCAGACTTCAGAAACAGAGGCATGACTGCCCTGCTG |
| | AAGGTGAGCAGCTGTGACAAGAACACAGGTGACTACTATGAGGACAGCTATGAAGACATCTCTGCCTACCTGCTGAGCAAGAACAATGCTATAGAACCCAGAAGCTTCAGCCAGAACCCCCCTGTGCTGAAGAGACACCAGAGAG |
| | AGATCACCAGAACCACCCTGCAGTCAGATCAGGAGGAGATAGATTATGATGATACCATCTCAGTAGAAATGAAGAAGGAGGACTTTGACATCTATGATGAAGATGAAAACCAGAGCCCCAGAAGCTTCCAGAAGAAGACCAGACA |
| | CTACTTCATAGCTGCTGTAGAGAGACTGTGGGACTATGGCATGAGCAGCAGCCCCCATGTACTGAGAAACAGAGCCCAGAGTGGCTCAGTGCCCCAGTTCAAGAAGGTGGTGTTCCAGGAGTTCACTGATGGCAGCTTCACCCAG |
| | CCCCTGTACAGAGGTGAACTGAATGAGCACCTGGGCCTGCTGGGCCCCTACATCAGAGCAGAGGTGGAGGACAACATCATGGTGACCTTCAGAAACCAGGCCAGCAGACCCTACAGCTTCTACAGCAGCCTGATCAGCTATGAAG |
| | AGGACCAGAGACAGGGTGCTGAGCCCAGAAAGAACTTTGTGAAGCCCAATGAGACCAAGACCTACTTCTGGAAGGTGCAGCACCACATGGCCCCCACCAAGGATGAGTTTGACTGCAAGGCCTGGGCCTACTTCAGTGATGTAGA |
| | CCTGGAGAAGGATGTGCACAGTGGGCTGATAGGTCCCCTGCTGGTGTGCCACACCAACACCCTGAACCCAGCACATGGTAGACAGGTGACAGTTCAGGAGTTTGCCCTGTTCTTCACCATCTTTGATGAAACCAAGAGCTGGTAC |
| | TTCACTGAGAACATGGAGAGAAACTGCAGAGCCCCCTGCAACATCCAGATGGAGGACCCCACCTTCAAGGAGAACTACAGATTCCATGCCATCAATGGCTACATCATGGACACCCTGCCAGGCCTGGTGATGGCCCAGGACCAGA |
| | GAATCAGATGGTACCTGCTGAGCATGGGCAGCAATGAGAACATCCACAGCATCCACTTCTCAGGACATGTGTTCACTGTGAGAAAGAAGGAGGAGTACAAGATGGCCCTGTACAACCTGTACCCAGGGGTATTTGAAACAGTAGA |
| | GATGCTGCCCAGCAAGGCAGGCATCTGGAGAGTGGAGTGCCTGATTGGAGAACACCTGCATGCAGGTATGAGCACCCTGTTCCTGGTGTACAGCAACAAGTGCCAGACCCCCCTGGGCATGGCCTCAGGCCACATCAGAGACTTC |
| | CAGATCACAGCTTCTGGCCAGTATGGGCAGTGGGCCCCCAAGCTGGCCAGACTGCACTACTCAGGTAGCATCAATGCTTGGAGCACCAAGGAGCCCTTCAGCTGGATCAAGGTGGACCTGCTGGCCCCCATGATCATCCATGGGA |
| | TCAAGACCCAGGGAGCCAGACAGAAGTTCAGCAGCCTGTACATCAGCCAGTTCATCATCATGTACAGCCTGGATGGTAAGAAGTGGCAGACCTACAGAGGCAACAGCACTGGCACCCTGATGGTGTTCTTTGGAAATGTTGATAG |
| | CTCAGGGATCAAGCACAACATCTTCAACCCCCCCATCATAGCCAGATACATCAGACTGCACCCCACCCACTACAGCATCAGAAGCACCCTGAGAATGGAGCTGATGGGCTGTGATCTGAACAGCTGCAGCATGCCCCTGGGCATG |
| | GAGAGCAAGGCCATCTCTGATGCACAGATCACAGCAAGCAGCTACTTCACCAACATGTTTGCCACCTGGAGCCCCAGCAAGGCCAGACTGCACCTGCAGGGCAGAAGCAATGCTTGGAGACCCCAGGTGAACAACCCCAAGGAGT |
| | GGCTGCAGGTGGACTTCCAGAAGACCATGAAGGTGACAGGGGTGACCACCCAGGGGGTGAAGAGCCTGCTGACCAGCATGTATGTCAAGGAGTTCCTGATCAGCAGCAGCCAGGATGGCCACCAGTGGACCCTGTTCTTCCAGAA |
| | TGGGAAGGTGAAGGTGTTCCAGGGCAACCAGGACAGCTTCACCCCAGTAGTGAACAGCCTGGACCCCCCCCTGCTGACCAGATACCTGAGAATCCACCCCCAGAGCTGGGTGCACCAGATTGCTCTGAGAATGGAGGTGCTGGGC |
| | TGTGAGGCCCAGGACCTGTACTGA |
| 12 | FVIII-QQ12 |
| | ATGCAGATAGAACTGAGCACCTGCTTCTTCCTGTGCCTGCTGAGATTCTGCTTCTCTGCCACCAGAAGATACTACCTGGGGGCTGTGGAGCTGAGCTGGGACTACATGCAGTCTGACCTGGGAGAACTGCCTGTTGATGCTAGAT |
| | TCCCCCCCAGAGTGCCCAAGAGCTTCCCCTTCAACACCTCAGTGGTGTACAAGAAGACCCTGTTTGTAGAGTTCACTGACCACCTGTTCAACATTGCAAAGCCCAGACCCCCCTGGATGGGCCTGCTGGGCCCCACCATCCAGGC |
| | TGAGGTGTATGATACTGTTGTGATCACCCTGAAGAACATGGCCAGCCACCCTGTGAGCCTGCATGCTGTGGGAGTCAGCTACTGGAAGGCCTCAGAGGGGGCTGAGTATGATGACCAGACCAGCCAGAGAGAGAAGGAGGATGAC |
| | AAGGTGTTCCCTGGTGGGAGCCACACCTATGTGTGGCAGGTGCTGAAGGAGAATGGACCCATGGCCTCAGACCCCCTGTGCCTGACCTACAGCTACCTGAGCCATGTAGACCTGGTGAAGGACCTGAACAGTGGTCTGATAGGTG |
| | CACTGCTGGTGTGCAGAGAGGGCAGCCTGGCCAAGGAGAAGACCCAGACCCTGCACAAGTTCATCCTGCTGTTTGCAGTGTTTGATGAGGGCAAGAGCTGGCACTCTGAGACCAAGAACAGCCTGATGCAGGACAGAGATGCTGC |
| | CTCAGCCAGAGCCTGGCCCAAGATGCACACTGTTAATGGGTATGTGAACAGAAGCCTGCCAGGCCTGATTGGCTGCCACAGAAAGAGTGTGTACTGGCATGTCATAGGAATGGGCACCACCCCTGAGGTGCACAGCATCTTCCTG |
| | GAGGGCCACACCTTCCTGGTGAGAAACCACAGACAGGCCAGCCTGGAGATCAGCCCCATCACCTTCCTGACTGCACAGACCCTGCTGATGGACCTGGGCCAGTTCCTGCTGTTCTGCCACATCAGCAGCCACCAGCATGATGGGA |
| | TGGAGGCCTATGTAAAGGTGGACAGCTGCCCTGAAGAGCCCCAGCTGCAGATGAAGAACAATGAGGAGGCAGAAGACTATGATGATGATCTGACAGACAGTGAAATGGATGTGGTGAGATTTGATGATGACAACAGCCCCAGCTT |
| | CATCCAGATCAGAAGTGTGGCCAAGAAGCACCCCAAGACCTGGGTGCACTACATTGCTGCAGAGGAGGAGGACTGGGACTATGCCCCCCTGGTGCTGGCCCCAGATGACAGAAGCTACAAGAGCCAGTACCTGAACAATGGGCCC |
| | CAGAGAATAGGGAGAAAGTACAAGAAGGTGAGATTCATGGCCTACACAGATGAGACCTTCAAGACCAGAGAGGCCATCCAGCATGAGAGTGGTATCCTGGGCCCCCTGCTGTATGGAGAAGTGGGGGATACCCTGCTGATCATCT |
| | TCAAGAACCAGGCCAGCAGACCCTACAACATCTACCCCCATGGCATCACAGATGTGAGACCCCTGTACAGCAGAAGACTGCCCAAGGGTGTAAAGCACCTGAAGGACTTCCCCATCCTGCCAGGGGAAATCTTCAAGTACAAGTG |
| | GACTGTGACTGTGGAGGATGGACCCACCAAGAGTGATCCCAGATGCCTGACCAGATACTACAGCAGCTTTGTTAACATGGAGAGAGACCTGGCCTCAGGACTGATTGGCCCCCTGCTGATCTGCTACAAGGAGAGTGTGGACCAG |
| | CAGGGCAACCAGATCATGAGTGACAAGAGAAATGTCATCCTGTTCAGTGTCTTTGATGAAAACAGAAGCTGGTACCTGACAGAAAACATCCAGAGATTCCTGCCCAACCCTGCAGGTGTGCAGCTGGAGGACCCAGAGTTCCAGG |
| | CCAGCAACATCATGCACAGCATCAATGGGTATGTGTTTGATAGCCTGCAGCTGAGTGTATGCCTGCATGAGGTGGCCTACTGGTACATCCTGAGCATAGGTGCTCAGACTGACTTCCTGTCAGTATTCTTCTCAGGCTACACCTT |
| | CAAGCACAAGATGGTGTATGAGGACACCCTGACCCTGTTCCCCTTCAGTGGGGAAACAGTGTTCATGAGCATGGAGAACCCTGGGCTGTGGATCCTGGGCTGCCACAACAGTGACTTCAGAAACAGAGGCATGACAGCCCTGCTG |
| | AAGGTGAGCAGCTGTGACAAGAACACAGGAGACTACTATGAAGACAGCTATGAAGACATCAGTGCTTACCTGCTGAGCAAGAACAATGCAATAGAGCCCAGAAGCTTCAGCCAGAACCCCCCAGTGCTGAAGAGACACCAGAGAG |
| | AGATCACCAGAACCACCCTGCAGTCAGACCAGGAGGAGATTGACTATGATGACACCATCTCTGTGGAGATGAAGAAGGAGGACTTTGACATCTATGATGAGGATGAGAACCAGAGCCCCAGAAGCTTCCAGAAGAAGACCAGACA |
| | CTACTTCATAGCAGCAGTGGAGAGACTGTGGGACTATGGGATGAGCAGCAGCCCCCATGTCCTGAGAAACAGAGCCCAGTCAGGAAGTGTCCCCCAGTTCAAGAAGGTGGTGTTCCAGGAGTTCACTGATGGGAGCTTCACCCAG |
| | CCCCTGTACAGAGGAGAGCTGAATGAACACCTGGGCCTGCTGGGCCCCTACATCAGAGCAGAGGTGGAGGACAACATCATGGTGACCTTCAGAAACCAGGCCAGCAGACCCTACAGCTTCTACAGCAGCCTGATCAGCTATGAGG |
| | AGGACCAGAGACAGGGAGCTGAACCCAGAAAGAACTTTGTGAAGCCCAATGAAACCAAGACCTACTTCTGGAAGGTGCAGCACCACATGGCCCCCACCAAGGATGAGTTTGACTGCAAGGCCTGGGCCTACTTCTCTGATGTTGA |
| | TCTGGAGAAGGATGTACACTCTGGGCTGATTGGCCCCCTGCTGGTGTGCCACACCAACACCCTGAACCCAGCCCATGGCAGACAGGTGACTGTGCAGGAGTTTGCCCTGTTCTTCACCATCTTTGATGAGACCAAGAGCTGGTAC |
| | TTCACTGAAAACATGGAGAGAAACTGCAGAGCCCCCTGCAACATCCAGATGGAGGACCCCACCTTCAAGGAGAACTACAGATTCCATGCTATCAATGGCTACATCATGGACACCCTGCCTGGACTGGTGATGGCCCAGGACCAGA |
| | GAATCAGATGGTACCTGCTGAGCATGGGCAGCAATGAAAACATCCACAGCATCCACTTCTCAGGCCATGTATTCACTGTGAGAAAGAAGGAGGAGTACAAGATGGCCCTGTACAACCTGTACCCAGGTGTGTTTGAAACAGTGGA |
| | AATGCTGCCCAGCAAGGCTGGCATCTGGAGAGTGGAGTGCCTGATTGGTGAGCACCTGCATGCTGGAATGAGCACCCTGTTCCTGGTGTACAGCAACAAGTGCCAGACCCCCCTGGGCATGGCCTCAGGGCACATCAGAGACTTC |
| | CAGATCACTGCCTCTGGCCAGTATGGACAGTGGGCCCCCAAGCTGGCCAGACTGCACTACAGTGGCAGCATCAATGCCTGGAGCACCAAGGAGCCCTTCAGCTGGATCAAGGTGGACCTGCTGGCCCCCATGATCATCCATGGCA |
| | TCAAGACCCAGGGTGCCAGACAGAAGTTCAGCAGCCTGTACATCAGCCAGTTCATCATCATGTACAGCCTGGATGGCAAGAAGTGGCAGACCTACAGAGGCAACAGCACAGGTACCCTGATGGTGTTCTTTGGCAATGTGGACAG |
| | CTCTGGTATCAAGCACAACATCTTCAACCCCCCCATCATAGCCAGATACATCAGACTGCACCCCACCCACTACAGCATCAGAAGCACCCTGAGAATGGAGCTGATGGGCTGTGATCTGAACAGCTGCAGCATGCCCCTGGGCATG |
| | GAGAGCAAGGCCATCTCTGATGCTCAGATCACAGCTAGCAGCTACTTCACCAACATGTTTGCCACCTGGAGCCCCAGCAAGGCCAGACTGCACCTGCAGGGCAGAAGCAATGCATGGAGACCCCAGGTGAACAACCCCAAGGAGT |
| | GGCTGCAGGTGGACTTCCAGAAGACCATGAAGGTGACAGGAGTGACCACCCAGGGAGTGAAGAGCCTGCTGACCAGCATGTATGTGAAGGAGTTCCTGATCAGCAGCAGCCAGGATGGCCACCAGTGGACCCTGTTCTTCCAGAA |
| | TGGGAAGGTGAAGGTGTTCCAGGGCAACCAGGACAGCTTCACCCCAGTAGTGAACAGCCTGGACCCCCCCCTGCTGACCAGATACCTGAGAATCCACCCCCAGAGCTGGGTGCACCAGATAGCTCTGAGAATGGAGGTGCTGGGC |
| | TGTGAAGCCCAGGACCTGTACTGA |
| 13 | FVIII-QQ13 |
| | ATGCAGATAGAACTGAGCACCTGCTTCTTCCTGTGCCTGCTGAGATTCTGCTTCAGTGCCACCAGAAGATACTACCTGGGTGCTGTTGAGCTGAGCTGGGACTACATGCAGTCTGACCTGGGTGAACTGCCAGTAGATGCAAGAT |
| | TCCCCCCCAGAGTGCCCAAGAGCTTCCCCTTCAACACCTCTGTAGTCTACAAGAAGACCCTGTTTGTGGAGTTCACAGATCACCTGTTCAACATTGCTAAGCCCAGACCCCCCTGGATGGGCCTGCTGGGCCCCACCATCCAGGC |
| | AGAGGTGTATGACACTGTAGTGATCACCCTGAAGAACATGGCCAGCCACCCAGTGAGCCTGCATGCTGTGGGTGTGAGCTACTGGAAGGCCAGTGAAGGAGCAGAGTATGATGATCAGACCAGCCAGAGAGAGAAGGAGGATGAC |
| | AAGGTGTTCCCAGGTGGAAGCCACACCTATGTCTGGCAGGTGCTGAAGGAGAATGGGCCCATGGCCTCAGATCCCCTGTGCCTGACCTACAGCTACCTGAGCCATGTTGACCTGGTGAAGGACCTGAACAGTGGACTGATAGGTG |
| | CTCTGCTGGTGTGCAGAGAGGGCAGCCTGGCCAAGGAGAAGACCCAGACCCTGCACAAGTTCATCCTGCTGTTTGCAGTGTTTGATGAAGGCAAGAGCTGGCACAGTGAGACCAAGAACAGCCTGATGCAGGACAGAGATGCAGC |
| | ATCAGCCAGAGCCTGGCCCAAGATGCACACAGTGAATGGCTATGTGAACAGAAGCCTGCCAGGCCTGATAGGATGCCACAGAAAGTCTGTATACTGGCATGTCATTGGTATGGGCACCACCCCTGAAGTGCACAGCATCTTCCTG |
| | GAGGGCCACACCTTCCTGGTGAGAAACCACAGACAGGCCAGCCTGGAGATCAGCCCCATCACCTTCCTGACAGCCCAGACCCTGCTGATGGACCTGGGCCAGTTCCTGCTGTTCTGCCACATCAGCAGCCACCAGCATGATGGGA |
| | TGGAGGCCTATGTCAAGGTGGACAGCTGCCCAGAGGAGCCCCAGCTGCAGATGAAGAACAATGAGGAGGCTGAGGACTATGATGATGACCTGACTGACTCAGAGATGGATGTGGTGAGATTTGATGATGATAACAGCCCCAGCTT |
| | CATCCAGATCAGATCTGTGGCCAAGAAGCACCCCAAGACCTGGGTGCACTACATAGCTGCAGAAGAGGAGGACTGGGACTATGCCCCCCTGGTGCTGGCCCCAGATGATAGAAGCTACAAGAGCCAGTACCTGAACAATGGCCCC |
| | CAGAGAATTGGGAGAAAGTACAAGAAGGTGAGATTCATGGCCTACACAGATGAAACCTTCAAGACCAGAGAGGCCATCCAGCATGAGTCTGGCATCCTGGGCCCCCTGCTGTATGGGGAAGTGGGAGACACCCTGCTGATCATCT |
| | TCAAGAACCAGGCCAGCAGACCCTACAACATCTACCCCCATGGTATCACAGATGTGAGACCCCTGTACAGCAGAAGACTGCCCAAGGGGGTGAAGCACCTGAAGGACTTCCCCATCCTGCCTGGGGAGATCTTCAAGTACAAGTG |
| | GACAGTAACTGTGGAGGATGGCCCCACCAAGTCTGACCCCAGATGCCTGACCAGATACTACAGCAGCTTTGTGAACATGGAGAGAGACCTGGCCAGTGGCCTGATTGGCCCCCTGCTGATCTGCTACAAGGAGAGTGTTGACCAG |
| | CAGGGCAACCAGATCATGAGTGATAAGAGAAATGTTATCCTGTTCAGTGTGTTTGATGAAAACAGAAGCTGGTACCTGACAGAGAACATCCAGAGATTCCTGCCCAACCCAGCTGGTGTCCAGCTGGAGGACCCTGAGTTCCAGG |
| | CCAGCAACATCATGCACAGCATCAATGGCTATGTATTTGACAGCCTGCAGCTGAGTGTGTGCCTGCATGAAGTGGCCTACTGGTACATCCTGAGCATAGGTGCTCAGACAGACTTCCTGAGTGTCTTCTTCAGTGGCTACACCTT |
| | CAAGCACAAGATGGTGTATGAAGACACCCTGACCCTGTTCCCCTTCAGTGGAGAGACAGTGTTCATGAGCATGGAGAACCCAGGACTGTGGATCCTGGGCTGCCACAACAGTGACTTCAGAAACAGAGGCATGACAGCCCTGCTG |
| | AAGGTGAGCAGCTGTGACAAGAACACTGGGGACTACTATGAGGACAGCTATGAGGACATCTCTGCCTACCTGCTGAGCAAGAACAATGCAATAGAGCCCAGAAGCTTCAGCCAGAACCCCCCTGTGCTGAAGAGACACCAGAGAG |
| | AGATCACCAGAACCACCCTGCAGTCAGATCAGGAGGAGATAGACTATGATGATACCATCTCTGTGGAGATGAAGAAGGAGGACTTTGATATCTATGATGAGGATGAGAACCAGAGCCCCAGAAGCTTCCAGAAGAAGACCAGACA |
| | CTACTTCATAGCTGCAGTGGAGAGACTGTGGGACTATGGCATGAGCAGCAGCCCCCATGTACTGAGAAACAGAGCCCAGTCAGGATCTGTCCCCCAGTTCAAGAAGGTGGTGTTCCAGGAGTTCACTGATGGGAGCTTCACCCAG |
| | CCCCTGTACAGAGGTGAACTGAATGAACACCTGGGCCTGCTGGGCCCCTACATCAGAGCAGAGGTGGAGGACAACATCATGGTGACCTTCAGAAACCAGGCCAGCAGACCCTACAGCTTCTACAGCAGCCTGATCAGCTATGAAG |
| | AGGACCAGAGACAGGGAGCTGAGCCCAGAAAGAACTTTGTGAAGCCCAATGAAACCAAGACCTACTTCTGGAAGGTGCAGCACCACATGGCCCCCACCAAGGATGAATTTGACTGCAAGGCCTGGGCCTACTTCTCAGATGTAGA |
| | CCTGGAGAAGGATGTGCACTCAGGACTGATTGGCCCCCTGCTGGTGTGCCACACCAACACCCTGAACCCAGCACATGGCAGACAGGTGACAGTTCAGGAGTTTGCACTGTTCTTCACCATCTTTGATGAGACCAAGAGCTGGTAC |
| | TTCACAGAGAACATGGAGAGAAACTGCAGAGCCCCCTGCAACATCCAGATGGAGGACCCCACCTTCAAGGAGAACTACAGATTCCATGCTATCAATGGCTACATCATGGACACCCTGCCAGGGCTGGTGATGGCCCAGGACCAGA |
| | GAATCAGATGGTACCTGCTGAGCATGGGCAGCAATGAAAACATCCACAGCATCCACTTCAGTGGCCATGTGTTCACAGTTAGAAAGAAGGAGGAGTACAAGATGGCCCTGTACAACCTGTACCCTGGTGTGTTTGAAACAGTGGA |
| | GATGCTGCCCAGCAAGGCTGGCATCTGGAGAGTGGAGTGCCTGATTGGGGAACACCTGCATGCAGGAATGAGCACCCTGTTCCTGGTGTACAGCAACAAGTGCCAGACCCCCCTGGGCATGGCCTCAGGCCACATCAGAGACTTC |
| | CAGATCACTGCCAGTGGGCAGTATGGTCAGTGGGCCCCCAAGCTGGCCAGACTGCACTACTCTGGCAGCATCAATGCCTGGAGCACCAAGGAGCCCTTCAGCTGGATCAAGGTGGACCTGCTGGCCCCCATGATCATCCATGGCA |
| | TCAAGACCCAGGGGGCCAGACAGAAGTTCAGCAGCCTGTACATCAGCCAGTTCATCATCATGTACAGCCTGGATGGCAAGAAGTGGCAGACCTACAGAGGCAACAGCACTGGAACCCTGATGGTGTTCTTTGGCAATGTAGACAG |
| | CTCAGGCATCAAGCACAACATCTTCAACCCCCCCATCATTGCCAGATACATCAGACTGCACCCCACCCACTACAGCATCAGAAGCACCCTGAGAATGGAGCTGATGGGCTGTGACCTGAACAGCTGCAGCATGCCCCTGGGCATG |
| | GAGAGCAAGGCCATCTCTGATGCCCAGATCACTGCCAGCAGCTACTTCACCAACATGTTTGCCACCTGGAGCCCCAGCAAGGCCAGACTGCACCTGCAGGGCAGAAGCAATGCTTGGAGACCCCAGGTGAACAACCCCAAGGAGT |
| | GGCTGCAGGTGGACTTCCAGAAGACCATGAAGGTGACTGGTGTGACCACCCAGGGTGTGAAGAGCCTGCTGACCAGCATGTATGTCAAGGAGTTCCTGATCAGCAGCAGCCAGGATGGTCACCAGTGGACCCTGTTCTTCCAGAA |
| | TGGCAAGGTGAAGGTGTTCCAGGGCAACCAGGACAGCTTCACCCCAGTGGTGAACAGCCTGGACCCCCCCCTGCTGACCAGATACCTGAGAATCCACCCCCAGAGCTGGGTGCACCAGATAGCCCTGAGAATGGAGGTGCTGGGC |
| | TGTGAGGCCCAGGACCTGTACTGA |
| 14 | FVIII-QQ14 |
| | ATGCAGATTGAACTGAGCACCTGCTTCTTCCTGTGCCTGCTGAGATTCTGCTTCAGTGCCACCAGAAGATACTACCTGGGGGCAGTGGAGCTGAGCTGGGACTACATGCAGAGTGACCTGGGGGAACTGCCTGTTGATGCCAGAT |
| | TCCCCCCCAGAGTGCCCAAGAGCTTCCCCTTCAACACCTCAGTTGTGTACAAGAAGACCCTGTTTGTGGAGTTCACTGACCACCTGTTCAACATTGCTAAGCCCAGACCCCCCTGGATGGGCCTGCTGGGCCCCACCATCCAGGC |
| | TGAGGTGTATGACACTGTGGTGATCACCCTGAAGAACATGGCCAGCCACCCTGTGAGCCTGCATGCAGTGGGGGTGAGCTACTGGAAGGCCTCAGAAGGGGCAGAGTATGATGATCAGACCAGCCAGAGAGAGAAGGAGGATGAC |
| | AAGGTGTTCCCTGGTGGTAGCCACACCTATGTGTGGCAGGTGCTGAAGGAGAATGGCCCCATGGCCTCAGACCCCCTGTGCCTGACCTACAGCTACCTGAGCCATGTGGACCTGGTGAAGGACCTGAACAGTGGCCTGATTGGAG |
| | CTCTGCTGGTGTGCAGAGAGGGCAGCCTGGCCAAGGAGAAGACCCAGACCCTGCACAAGTTCATCCTGCTGTTTGCTGTGTTTGATGAGGGCAAGAGCTGGCACTCAGAGACCAAGAACAGCCTGATGCAGGACAGAGATGCTGC |
| | CAGTGCAAGAGCCTGGCCCAAGATGCACACTGTCAATGGATATGTGAACAGAAGCCTGCCAGGACTGATAGGCTGCCACAGAAAGTCTGTATACTGGCATGTTATAGGTATGGGCACCACCCCAGAGGTGCACAGCATCTTCCTG |
| | GAGGGCCACACCTTCCTGGTGAGAAACCACAGACAGGCCAGCCTGGAGATCAGCCCCATCACCTTCCTGACTGCCCAGACCCTGCTGATGGACCTGGGCCAGTTCCTGCTGTTCTGCCACATCAGCAGCCACCAGCATGATGGTA |
| | TGGAGGCCTATGTGAAGGTGGACAGCTGCCCTGAGGAGCCCCAGCTGCAGATGAAGAACAATGAGGAGGCAGAGGACTATGATGATGACCTGACAGACAGTGAGATGGATGTGGTGAGATTTGATGATGACAACAGCCCCAGCTT |
| | CATCCAGATCAGAAGTGTGGCCAAGAAGCACCCCAAGACCTGGGTGCACTACATTGCTGCTGAAGAGGAGGACTGGGACTATGCACCCCTGGTGCTGGCCCCAGATGATAGAAGCTACAAGAGCCAGTACCTGAACAATGGACCC |
| | CAGAGAATTGGAAGAAAGTACAAGAAGGTGAGATTCATGGCCTACACTGATGAGACCTTCAAGACCAGAGAGGCCATCCAGCATGAATCAGGGATCCTGGGCCCCCTGCTGTATGGAGAGGTGGGAGACACCCTGCTGATCATCT |
| | TCAAGAACCAGGCCAGCAGACCCTACAACATCTACCCCCATGGTATCACTGATGTAAGACCCCTGTACAGCAGAAGACTGCCCAAGGGAGTGAAGCACCTGAAGGACTTCCCCATCCTGCCTGGGGAAATCTTCAAGTACAAGTG |
| | GACAGTCACAGTTGAAGATGGACCCACCAAGAGTGATCCCAGATGCCTGACCAGATACTACAGCAGCTTTGTGAACATGGAGAGAGACCTGGCCTCAGGCCTGATAGGCCCCCTGCTGATCTGCTACAAGGAGTCTGTTGACCAG |
| | CAGGGCAACCAGATCATGAGTGACAAGAGAAATGTGATCCTGTTCTCAGTGTTTGATGAGAACAGAAGCTGGTACCTGACTGAAAACATCCAGAGATTCCTGCCCAACCCAGCAGGGGTGCAGCTGGAGGACCCAGAGTTCCAGG |
| | CCAGCAACATCATGCACAGCATCAATGGCTATGTCTTTGATAGCCTGCAGCTGTCTGTATGCCTGCATGAGGTGGCCTACTGGTACATCCTGAGCATAGGAGCCCAGACTGATTTCCTGAGTGTTTTCTTCAGTGGCTACACCTT |
| | CAAGCACAAGATGGTGTATGAGGACACCCTGACCCTGTTCCCCTTCTCTGGTGAGACTGTGTTCATGAGCATGGAGAACCCTGGTCTGTGGATCCTGGGCTGCCACAACTCAGATTTCAGAAACAGAGGCATGACAGCCCTGCTG |
| | AAGGTGAGCAGCTGTGACAAGAACACTGGGGACTACTATGAAGACAGCTATGAAGACATCTCTGCTTACCTGCTGAGCAAGAACAATGCCATTGAGCCCAGAAGCTTCAGCCAGAACCCCCCTGTGCTGAAGAGACACCAGAGAG |
| | AGATCACCAGAACCACCCTGCAGTCAGACCAGGAGGAGATAGACTATGATGATACCATCTCTGTGGAGATGAAGAAGGAGGACTTTGATATCTATGATGAGGATGAAAACCAGAGCCCCAGAAGCTTCCAGAAGAAGACCAGACA |
| | CTACTTCATAGCAGCTGTAGAGAGACTGTGGGACTATGGCATGAGCAGCAGCCCCCATGTACTGAGAAACAGAGCCCAGAGTGGAAGTGTGCCCCAGTTCAAGAAGGTGGTGTTCCAGGAGTTCACAGATGGCAGCTTCACCCAG |
| | CCCCTGTACAGAGGAGAACTGAATGAGCACCTGGGCCTGCTGGGCCCCTACATCAGAGCTGAGGTGGAGGACAACATCATGGTGACCTTCAGAAACCAGGCCAGCAGACCCTACAGCTTCTACAGCAGCCTGATCAGCTATGAGG |
| | AGGACCAGAGACAGGGAGCTGAGCCCAGAAAGAACTTTGTCAAGCCCAATGAGACCAAGACCTACTTCTGGAAGGTGCAGCACCACATGGCCCCCACCAAGGATGAATTTGACTGCAAGGCCTGGGCCTACTTCTCAGATGTGGA |
| | CCTGGAGAAGGATGTGCACTCAGGTCTGATTGGCCCCCTGCTGGTGTGCCACACCAACACCCTGAACCCAGCCCATGGTAGACAGGTGACTGTGCAGGAGTTTGCTCTGTTCTTCACCATCTTTGATGAGACCAAGAGCTGGTAC |
| | TTCACAGAGAACATGGAGAGAAACTGCAGAGCCCCCTGCAACATCCAGATGGAGGACCCCACCTTCAAGGAGAACTACAGATTCCATGCCATCAATGGGTACATCATGGACACCCTGCCAGGTCTGGTGATGGCCCAGGACCAGA |
| | GAATCAGATGGTACCTGCTGAGCATGGGCAGCAATGAAAACATCCACAGCATCCACTTCAGTGGACATGTGTTCACAGTTAGAAAGAAGGAGGAGTACAAGATGGCCCTGTACAACCTGTACCCTGGAGTGTTTGAAACTGTGGA |
| | GATGCTGCCCAGCAAGGCAGGCATCTGGAGAGTGGAGTGCCTGATAGGGGAGCACCTGCATGCTGGTATGAGCACCCTGTTCCTGGTGTACAGCAACAAGTGCCAGACCCCCCTGGGCATGGCCTCAGGCCACATCAGAGACTTC |
| | CAGATCACTGCTAGTGGCCAGTATGGTCAGTGGGCCCCCAAGCTGGCCAGACTGCACTACTCTGGAAGCATCAATGCCTGGAGCACCAAGGAGCCCTTCAGCTGGATCAAGGTGGACCTGCTGGCCCCCATGATCATCCATGGGA |
| | TCAAGACCCAGGGGGCCAGACAGAAGTTCAGCAGCCTGTACATCAGCCAGTTCATCATCATGTACAGCCTGGATGGAAAGAAGTGGCAGACCTACAGAGGCAACAGCACAGGCACCCTGATGGTGTTCTTTGGCAATGTAGACAG |
| | CTCAGGCATCAAGCACAACATCTTCAACCCCCCCATCATAGCAAGATACATCAGACTGCACCCCACCCACTACAGCATCAGAAGCACCCTGAGAATGGAGCTGATGGGCTGTGACCTGAACAGCTGCAGCATGCCCCTGGGCATG |
| | GAGAGCAAGGCCATCTCAGATGCCCAGATCACAGCCAGCAGCTACTTCACCAACATGTTTGCCACCTGGAGCCCCAGCAAGGCCAGACTGCACCTGCAGGGCAGAAGCAATGCCTGGAGACCCCAGGTGAACAACCCCAAGGAGT |
| | GGCTGCAGGTGGACTTCCAGAAGACCATGAAGGTGACTGGTGTGACCACCCAGGGTGTTAAGAGCCTGCTGACCAGCATGTATGTGAAGGAGTTCCTGATCAGCAGCAGCCAGGATGGGCACCAGTGGACCCTGTTCTTCCAGAA |
| | TGGAAAGGTGAAGGTGTTCCAGGGCAACCAGGACAGCTTCACCCCTGTTGTGAACAGCCTGGACCCCCCCCTGCTGACCAGATACCTGAGAATCCACCCCCAGAGCTGGGTGCACCAGATAGCACTGAGAATGGAGGTGCTGGGC |
| | TGTGAGGCCCAGGACCTGTACTGA |
| 15 | FVIII-QQ15 |
| | ATGCAGATTGAACTGAGCACCTGCTTCTTCCTGTGCCTGCTGAGATTCTGCTTCAGTGCTACCAGAAGATACTACCTGGGGGCAGTGGAGCTGAGCTGGGACTACATGCAGTCTGACCTGGGAGAGCTGCCAGTAGATGCCAGAT |
| | TCCCCCCCAGAGTGCCCAAGAGCTTCCCCTTCAACACCTCTGTGGTGTACAAGAAGACCCTGTTTGTGGAGTTCACAGACCACCTGTTCAACATAGCTAAGCCCAGACCCCCCTGGATGGGCCTGCTGGGCCCCACCATCCAGGC |
| | AGAGGTGTATGACACTGTTGTGATCACCCTGAAGAACATGGCCAGCCACCCAGTTAGCCTGCATGCTGTTGGTGTTAGCTACTGGAAGGCCTCTGAGGGAGCAGAGTATGATGACCAGACCAGCCAGAGAGAGAAGGAGGATGAC |
| | AAGGTGTTCCCAGGGGGGAGCCACACCTATGTCTGGCAGGTGCTGAAGGAGAATGGCCCCATGGCCTCAGACCCCCTGTGCCTGACCTACAGCTACCTGAGCCATGTGGACCTGGTGAAGGACCTGAACTCTGGCCTGATTGGTG |
| | CACTGCTGGTGTGCAGAGAGGGCAGCCTGGCCAAGGAGAAGACCCAGACCCTGCACAAGTTCATCCTGCTGTTTGCTGTCTTTGATGAGGGCAAGAGCTGGCACTCAGAAACCAAGAACAGCCTGATGCAGGACAGAGATGCAGC |
| | TTCTGCTAGAGCCTGGCCCAAGATGCACACTGTGAATGGCTATGTCAACAGAAGCCTGCCTGGCCTGATAGGCTGCCACAGAAAGTCTGTGTACTGGCATGTGATTGGGATGGGCACCACCCCAGAGGTGCACAGCATCTTCCTG |
| | GAGGGCCACACCTTCCTGGTGAGAAACCACAGACAGGCCAGCCTGGAGATCAGCCCCATCACCTTCCTGACTGCCCAGACCCTGCTGATGGACCTGGGCCAGTTCCTGCTGTTCTGCCACATCAGCAGCCACCAGCATGATGGTA |
| | TGGAGGCCTATGTGAAGGTGGACAGCTGCCCAGAGGAGCCCCAGCTGCAGATGAAGAACAATGAAGAGGCAGAGGACTATGATGATGACCTGACAGACTCAGAAATGGATGTGGTGAGATTTGATGATGATAACAGCCCCAGCTT |
| | CATCCAGATCAGATCTGTTGCCAAGAAGCACCCCAAGACCTGGGTGCACTACATTGCAGCTGAGGAGGAGGACTGGGACTATGCTCCCCTGGTGCTGGCCCCAGATGACAGAAGCTACAAGAGCCAGTACCTGAACAATGGACCC |
| | CAGAGAATAGGGAGAAAGTACAAGAAGGTGAGATTCATGGCCTACACAGATGAGACCTTCAAGACCAGAGAGGCCATCCAGCATGAATCAGGGATCCTGGGCCCCCTGCTGTATGGGGAGGTGGGTGACACCCTGCTGATCATCT |
| | TCAAGAACCAGGCCAGCAGACCCTACAACATCTACCCCCATGGAATCACAGATGTCAGACCCCTGTACAGCAGAAGACTGCCCAAGGGAGTAAAGCACCTGAAGGACTTCCCCATCCTGCCAGGTGAGATCTTCAAGTACAAGTG |
| | GACAGTAACAGTGGAGGATGGTCCCACCAAGTCTGACCCCAGATGCCTGACCAGATACTACAGCAGCTTTGTTAACATGGAGAGAGACCTGGCCAGTGGCCTGATAGGCCCCCTGCTGATCTGCTACAAGGAGTCTGTAGACCAG |
| | CAGGGCAACCAGATCATGTCAGATAAGAGAAATGTAATCCTGTTCAGTGTGTTTGATGAGAACAGAAGCTGGTACCTGACAGAGAACATCCAGAGATTCCTGCCCAACCCTGCAGGTGTGCAGCTGGAGGACCCTGAATTCCAGG |
| | CCAGCAACATCATGCACAGCATCAATGGATATGTTTTTGACAGCCTGCAGCTGTCAGTTTGCCTGCATGAGGTGGCCTACTGGTACATCCTGAGCATTGGGGCCCAGACAGATTTCCTGTCTGTGTTCTTCAGTGGGTACACCTT |
| | CAAGCACAAGATGGTGTATGAGGACACCCTGACCCTGTTCCCCTTCTCAGGTGAAACTGTGTTCATGAGCATGGAGAACCCAGGGCTGTGGATCCTGGGCTGCCACAACTCAGACTTCAGAAACAGAGGCATGACTGCTCTGCTG |
| | AAGGTGAGCAGCTGTGACAAGAACACAGGTGACTACTATGAGGACAGCTATGAAGACATCTCAGCTTACCTGCTGAGCAAGAACAATGCCATAGAGCCCAGAAGCTTCAGCCAGAACCCCCCAGTTCTGAAGAGACACCAGAGAG |
| | AGATCACCAGAACCACCCTGCAGAGTGACCAGGAGGAGATTGACTATGATGACACCATCTCTGTAGAGATGAAGAAGGAGGACTTTGACATCTATGATGAGGATGAGAACCAGAGCCCCAGAAGCTTCCAGAAGAAGACCAGACA |
| | CTACTTCATAGCTGCAGTGGAGAGACTGTGGGACTATGGCATGAGCAGCAGCCCCCATGTGCTGAGAAACAGAGCCCAGAGTGGCTCAGTGCCCCAGTTCAAGAAGGTGGTGTTCCAGGAGTTCACAGATGGCAGCTTCACCCAG |
| | CCCCTGTACAGAGGGGAGCTGAATGAGCACCTGGGCCTGCTGGGCCCCTACATCAGAGCTGAGGTGGAGGACAACATCATGGTGACCTTCAGAAACCAGGCCAGCAGACCCTACAGCTTCTACAGCAGCCTGATCAGCTATGAGG |
| | AGGACCAGAGACAGGGGGCAGAGCCCAGAAAGAACTTTGTAAAGCCCAATGAAACCAAGACCTACTTCTGGAAGGTGCAGCACCACATGGCCCCCACCAAGGATGAATTTGACTGCAAGGCCTGGGCCTACTTCAGTGATGTGGA |
| | CCTGGAGAAGGATGTGCACTCAGGCCTGATTGGTCCCCTGCTGGTGTGCCACACCAACACCCTGAACCCAGCCCATGGCAGACAGGTGACTGTACAGGAGTTTGCCCTGTTCTTCACCATCTTTGATGAGACCAAGAGCTGGTAC |
| | TTCACAGAGAACATGGAGAGAAACTGCAGAGCCCCCTGCAACATCCAGATGGAGGACCCCACCTTCAAGGAGAACTACAGATTCCATGCTATCAATGGGTACATCATGGACACCCTGCCAGGCCTGGTGATGGCCCAGGACCAGA |
| | GAATCAGATGGTACCTGCTGAGCATGGGCAGCAATGAGAACATCCACAGCATCCACTTCAGTGGCCATGTGTTCACAGTGAGAAAGAAGGAGGAGTACAAGATGGCCCTGTACAACCTGTACCCTGGAGTGTTTGAAACTGTGGA |
| | GATGCTGCCCAGCAAGGCAGGTATCTGGAGAGTGGAGTGCCTGATTGGTGAGCACCTGCATGCTGGGATGAGCACCCTGTTCCTGGTGTACAGCAACAAGTGCCAGACCCCCCTGGGCATGGCCTCAGGGCACATCAGAGACTTC |
| | CAGATCACTGCCAGTGGGCAGTATGGGCAGTGGGCCCCCAAGCTGGCCAGACTGCACTACAGTGGAAGCATCAATGCATGGAGCACCAAGGAGCCCTTCAGCTGGATCAAGGTGGACCTGCTGGCCCCCATGATCATCCATGGGA |
| | TCAAGACCCAGGGGGCCAGACAGAAGTTCAGCAGCCTGTACATCAGCCAGTTCATCATCATGTACAGCCTGGATGGCAAGAAGTGGCAGACCTACAGAGGCAACAGCACAGGTACCCTGATGGTGTTCTTTGGAAATGTGGACAG |
| | CTCAGGTATCAAGCACAACATCTTCAACCCCCCCATCATAGCAAGATACATCAGACTGCACCCCACCCACTACAGCATCAGAAGCACCCTGAGAATGGAGCTGATGGGCTGTGACCTGAACAGCTGCAGCATGCCCCTGGGCATG |
| | GAGAGCAAGGCCATCTCAGATGCTCAGATCACTGCCAGCAGCTACTTCACCAACATGTTTGCCACCTGGAGCCCCAGCAAGGCCAGACTGCACCTGCAGGGCAGAAGCAATGCCTGGAGACCCCAGGTGAACAACCCCAAGGAGT |
| | GGCTGCAGGTGGACTTCCAGAAGACCATGAAGGTGACAGGGGTTACCACCCAGGGAGTGAAGAGCCTGCTGACCAGCATGTATGTAAAGGAGTTCCTGATCAGCAGCAGCCAGGATGGTCACCAGTGGACCCTGTTCTTCCAGAA |
| | TGGAAAGGTGAAGGTGTTCCAGGGCAACCAGGACAGCTTCACCCCAGTGGTGAACAGCCTGGACCCCCCCCTGCTGACCAGATACCTGAGAATCCACCCCCAGAGCTGGGTGCACCAGATAGCCCTGAGAATGGAGGTGCTGGGC |
| | TGTGAAGCCCAGGACCTGTACTGA |
| 16 | FVIII-QQ16 |
| | ATGCAAATAGAACTCTCCACCTGCTTCTTTCTGTGCCTTTTGAGGTTCTGCTTTTCTGCAACTAGAAGATACTACCTGGGTGCAGTAGAACTGTCATGGGACTATATGCAATCAGATCTAGGAGAACTGCCAGTGGATGCCAGAT |
| | TTCCTCCAAGGGTGCCCAAATCATTTCCATTCAACACAAGTGTTGTGTACAAAAAGACCCTGTTTGTGGAATTCACAGATCACCTTTTCAACATAGCCAAGCCTAGACCACCTTGGATGGGTCTGTTGGGTCCTACCATCCAGGC |
| | AGAAGTTTATGATACTGTGGTCATTACACTTAAGAACATGGCTAGCCATCCAGTCAGTCTTCATGCTGTTGGGGTATCCTACTGGAAAGCTTCAGAGGGAGCAGAATATGATGACCAGACAAGTCAAAGGGAGAAAGAAGATGAC |
| | AAAGTCTTCCCTGGTGGAAGCCATACCTATGTCTGGCAGGTCCTGAAAGAGAATGGGCCAATGGCCAGTGACCCACTGTGCCTTACCTACTCTTATCTTAGTCATGTGGACCTGGTAAAAGACTTGAATAGTGGCCTCATTGGAG |
| | CACTACTGGTATGTAGAGAAGGGAGCTTGGCTAAGGAAAAGACACAGACTTTGCACAAATTTATACTACTTTTTGCTGTATTTGATGAGGGGAAAAGTTGGCACAGTGAAACAAAGAACTCCTTGATGCAGGATAGGGATGCAGC |
| | AAGTGCTAGGGCTTGGCCAAAAATGCACACAGTTAATGGGTATGTTAACAGATCACTGCCTGGGCTGATTGGTTGCCACAGAAAATCTGTCTATTGGCATGTGATTGGAATGGGCACAACTCCTGAAGTGCACTCAATATTCCTT |
| | GAAGGTCACACATTTCTTGTGAGAAACCATAGACAGGCTTCCTTGGAAATCAGCCCCATAACCTTCCTTACAGCTCAAACACTCTTGATGGACCTTGGACAGTTTCTACTGTTTTGTCATATCTCTTCCCACCAACATGATGGAA |
| | TGGAAGCCTATGTCAAAGTAGACAGCTGTCCTGAGGAACCACAACTACAGATGAAAAATAATGAAGAAGCAGAAGACTATGATGATGATCTTACTGATTCAGAAATGGATGTGGTCAGGTTTGATGATGATAACTCTCCTTCCTT |
| | TATCCAAATTAGATCTGTAGCAAAGAAGCATCCTAAAACTTGGGTACATTACATTGCTGCAGAAGAGGAGGACTGGGACTATGCACCCTTGGTCTTGGCACCAGATGATAGAAGTTATAAAAGTCAATATTTGAACAATGGCCCC |
| | CAGAGAATTGGTAGGAAGTACAAAAAAGTCAGATTTATGGCATACACAGATGAAACATTTAAGACAAGAGAAGCTATTCAGCATGAGTCAGGGATCTTGGGACCTTTACTTTATGGTGAAGTTGGAGATACACTGTTGATTATAT |
| | TTAAGAATCAAGCAAGCAGACCATATAACATCTACCCTCATGGTATCACAGATGTAAGACCCTTGTATTCAAGAAGATTACCCAAAGGTGTAAAACATTTGAAGGATTTTCCAATTCTGCCAGGGGAGATATTCAAATATAAATG |
| | GACAGTGACAGTAGAGGATGGGCCAACTAAATCAGATCCTAGGTGCCTGACCAGGTATTACTCTAGTTTTGTTAATATGGAGAGAGATCTAGCTAGTGGACTCATTGGACCTCTCCTCATCTGCTACAAAGAATCAGTTGATCAA |
| | CAGGGAAACCAGATAATGTCAGATAAGAGGAATGTCATCCTGTTTTCTGTATTTGATGAAAACAGGAGCTGGTACCTCACAGAAAATATACAAAGATTTCTCCCCAATCCTGCTGGAGTGCAGCTTGAAGATCCAGAATTCCAAG |
| | CTTCAAACATCATGCACAGCATCAATGGCTATGTATTTGATTCCTTGCAGTTGTCAGTGTGTTTGCATGAGGTGGCATACTGGTACATTCTATCTATTGGAGCTCAGACTGACTTCCTTTCTGTGTTCTTCTCAGGATATACATT |
| | CAAACACAAAATGGTCTATGAAGACACACTCACCCTATTCCCATTCTCAGGGGAAACAGTCTTCATGTCAATGGAAAACCCAGGTCTATGGATTTTGGGGTGCCACAACTCAGACTTTAGAAACAGAGGCATGACAGCTTTACTG |
| | AAGGTTAGTAGTTGTGATAAGAACACTGGGGATTATTATGAGGACTCTTATGAAGATATTTCTGCATACTTGCTGAGTAAAAACAATGCAATTGAACCAAGAAGCTTCTCTCAAAACCCCCCAGTCTTGAAAAGACATCAAAGAG |
| | AAATAACCAGAACTACTCTTCAGTCTGATCAAGAGGAAATTGACTATGATGATACCATATCAGTTGAAATGAAGAAGGAAGATTTTGACATTTATGATGAAGATGAAAATCAGAGCCCCAGGAGCTTTCAAAAGAAAACAAGACA |
| | CTATTTTATTGCAGCAGTGGAGAGGCTCTGGGATTATGGTATGAGTAGCTCCCCACATGTACTAAGGAACAGGGCCCAGAGTGGCTCTGTCCCACAGTTCAAGAAAGTAGTTTTCCAGGAATTTACAGATGGCTCCTTTACTCAG |
| | CCTTTATACAGGGGAGAACTAAATGAACATTTGGGACTCCTAGGGCCATATATAAGAGCAGAGGTTGAAGATAATATCATGGTAACTTTCAGGAATCAGGCCTCTAGGCCCTATTCCTTCTATTCTAGCCTTATTTCTTATGAGG |
| | AAGATCAGAGGCAAGGAGCAGAGCCTAGGAAAAACTTTGTTAAGCCTAATGAAACCAAAACATACTTTTGGAAAGTGCAACATCATATGGCCCCCACCAAAGATGAATTTGACTGCAAAGCCTGGGCATATTTCTCTGATGTGGA |
| | CTTAGAAAAAGATGTTCACTCAGGCCTGATAGGACCACTTCTGGTCTGCCACACTAACACACTGAACCCAGCCCATGGGAGACAAGTGACTGTACAGGAATTTGCACTGTTTTTCACCATCTTTGATGAAACAAAAAGCTGGTAC |
| | TTCACTGAAAATATGGAAAGAAACTGCAGGGCTCCCTGCAATATCCAGATGGAAGATCCAACATTTAAAGAGAATTATAGATTCCATGCTATCAATGGCTACATAATGGATACACTACCAGGCTTAGTAATGGCTCAGGATCAAA |
| | GGATTAGATGGTATCTGCTCAGCATGGGCAGTAATGAGAACATCCATTCTATTCATTTCTCAGGACATGTATTCACTGTAAGAAAAAAAGAGGAGTATAAAATGGCACTGTACAATCTCTATCCAGGTGTTTTTGAGACAGTAGA |
| | AATGTTACCTTCAAAAGCAGGAATTTGGAGAGTGGAATGCCTTATTGGTGAGCATCTACATGCTGGAATGAGCACACTTTTTCTGGTGTACAGCAATAAGTGTCAGACCCCTCTGGGAATGGCAAGTGGACACATTAGAGACTTT |
| | CAGATTACAGCTTCAGGACAATATGGACAGTGGGCCCCAAAGCTGGCCAGACTTCATTATTCAGGGTCAATCAATGCATGGAGCACCAAGGAGCCTTTTAGCTGGATCAAGGTGGATCTGTTGGCACCAATGATTATTCATGGCA |
| | TCAAGACTCAGGGAGCCAGACAGAAGTTCTCAAGCCTCTACATCTCTCAGTTTATCATCATGTATTCCCTTGATGGGAAGAAGTGGCAGACTTATAGAGGAAATAGTACAGGAACCTTAATGGTCTTCTTTGGGAATGTGGATTC |
| | AAGTGGGATAAAACACAATATTTTTAACCCTCCAATTATTGCTAGGTACATCAGGTTGCACCCCACTCATTATAGCATTAGGAGCACTCTTAGAATGGAGTTGATGGGCTGTGATTTAAATAGTTGCAGTATGCCATTGGGAATG |
| | GAGAGTAAAGCAATATCAGATGCACAGATTACTGCATCATCTTACTTTACTAATATGTTTGCAACTTGGTCTCCATCAAAAGCAAGACTTCACCTCCAAGGGAGGTCTAATGCTTGGAGACCTCAGGTGAATAATCCAAAAGAGT |
| | GGCTGCAAGTGGACTTCCAGAAGACAATGAAAGTCACAGGTGTGACCACTCAGGGTGTAAAATCTCTGCTTACCAGCATGTATGTTAAGGAGTTCCTCATCTCCAGCAGTCAAGATGGACATCAGTGGACTCTCTTTTTTCAGAA |
| | TGGTAAAGTAAAGGTTTTTCAGGGAAATCAAGACTCCTTCACACCAGTGGTTAACAGTCTAGACCCACCATTACTGACTAGGTACCTTAGAATTCACCCACAGAGTTGGGTGCACCAGATTGCCCTGAGGATGGAGGTTCTGGGC |
| | TGTGAAGCACAGGACCTCTACTGA |
| 17 | FVIII-QQ17 |
| | ATGCAAATAGAGCTCTCCACCTGCTTCTTTCTGTGCCTTTTGAGGTTCTGCTTTTCTGCCACAAGGAGATACTACCTGGGTGCAGTGGAACTGTCATGGGACTATATGCAAAGTGACCTAGGAGAGCTGCCAGTGGATGCAAGGT |
| | TTCCTCCTAGAGTGCCCAAAAGTTTTCCATTCAACACATCAGTTGTGTACAAAAAGACACTGTTTGTAGAATTCACAGATCACCTTTTCAACATAGCAAAGCCCAGGCCACCATGGATGGGTCTGCTAGGTCCTACCATCCAGGC |
| | AGAGGTTTATGATACTGTGGTCATTACACTTAAGAACATGGCTTCACATCCTGTCAGTCTTCATGCTGTGGGTGTATCCTACTGGAAAGCTTCAGAGGGAGCAGAATATGATGACCAGACTAGTCAAAGAGAGAAAGAAGATGAT |
| | AAAGTCTTCCCAGGTGGATCCCATACCTATGTCTGGCAGGTCCTGAAAGAGAATGGTCCTATGGCCAGTGATCCTCTGTGCCTTACTTACAGTTATCTTAGTCATGTTGACCTGGTAAAAGACTTGAATTCAGGACTCATTGGAG |
| | CACTATTGGTATGTAGAGAAGGGTCTTTAGCTAAGGAAAAGACACAGACCTTGCACAAATTTATACTACTTTTTGCAGTATTTGATGAAGGGAAAAGTTGGCACAGTGAAACAAAGAACTCCTTGATGCAGGATAGGGATGCAGC |
| | ATCTGCTAGGGCCTGGCCCAAAATGCACACAGTCAATGGGTATGTGAACAGGTCTCTGCCTGGTCTGATTGGATGCCACAGAAAATCAGTATATTGGCATGTGATAGGAATGGGCACAACTCCTGAGGTGCACTCAATATTCTTA |
| | GAAGGTCACACATTTCTTGTAAGAAACCATAGACAGGCATCCTTGGAAATCTCTCCCATAACATTCCTTACAGCTCAAACCCTCTTGATGGACCTTGGACAGTTTCTACTGTTTTGTCATATCTCTTCCCACCAACATGATGGGA |
| | TGGAAGCATATGTCAAAGTAGACAGCTGTCCAGAGGAACCACAACTACAGATGAAAAATAATGAAGAAGCAGAAGACTATGATGATGATCTTACTGATTCAGAAATGGATGTAGTCAGGTTTGATGATGATAACTCTCCTTCCTT |
| | TATCCAAATTAGGTCTGTTGCCAAGAAGCATCCTAAAACTTGGGTACATTACATTGCTGCAGAAGAGGAGGACTGGGACTATGCACCATTAGTACTAGCACCTGATGACAGAAGTTATAAAAGTCAATATTTGAACAATGGACCC |
| | CAGAGAATTGGGAGGAAGTACAAAAAAGTCAGATTTATGGCATACACTGATGAGACATTTAAGACTAGAGAAGCTATTCAGCATGAATCAGGAATCTTGGGACCTTTACTTTATGGGGAAGTAGGAGACACACTGTTGATTATAT |
| | TTAAGAATCAAGCAAGCAGACCATATAACATCTACCCACATGGAATCACAGATGTTAGACCCTTGTATTCAAGGAGGTTACCAAAAGGTGTAAAACATTTGAAGGATTTTCCTATTCTGCCAGGTGAAATATTCAAATATAAATG |
| | GACTGTGACTGTTGAAGATGGACCAACTAAATCAGACCCCAGGTGCCTGACCAGGTATTACTCTAGTTTTGTTAATATGGAGAGAGATCTAGCTTCAGGACTCATAGGCCCTCTCCTCATCTGCTACAAAGAATCAGTAGATCAA |
| | CAGGGAAACCAGATAATGTCAGACAAGAGGAATGTAATCCTGTTTTCTGTATTTGATGAAAACAGAAGCTGGTACCTCACAGAGAATATACAAAGGTTTCTCCCCAATCCTGCAGGAGTGCAGCTTGAGGATCCTGAGTTCCAAG |
| | CTTCCAACATCATGCACAGCATCAATGGGTATGTGTTTGATTCTTTGCAGTTGTCTGTTTGTTTGCATGAAGTGGCATACTGGTACATTCTATCTATTGGTGCTCAGACTGACTTCCTTTCTGTATTCTTCTCAGGCTATACTTT |
| | CAAACACAAAATGGTCTATGAAGACACCCTCACCCTATTCCCATTCTCTGGGGAAACTGTGTTCATGTCCATGGAAAACCCTGGACTATGGATTCTGGGCTGCCACAACTCTGACTTTAGAAACAGAGGCATGACTGCTTTACTG |
| | AAGGTTTCAAGTTGTGACAAGAACACTGGTGATTATTATGAAGACTCCTATGAAGATATTAGTGCCTACTTGCTGAGTAAAAACAATGCAATAGAACCAAGGAGCTTCTCTCAAAACCCCCCAGTCTTGAAAAGGCATCAAAGAG |
| | AAATAACCAGAACTACTCTTCAGTCAGATCAAGAGGAAATTGACTATGATGATACCATAAGTGTTGAAATGAAGAAGGAAGATTTTGACATTTATGATGAGGATGAAAATCAGAGCCCCAGGAGCTTTCAAAAGAAAACAAGACA |
| | CTATTTTATAGCTGCTGTAGAGAGACTCTGGGATTATGGGATGAGTTCATCCCCTCATGTACTAAGGAACAGGGCCCAGTCAGGTTCAGTCCCCCAGTTCAAGAAAGTTGTCTTCCAGGAATTTACAGATGGGTCCTTTACTCAG |
| | CCTTTATACAGGGGAGAACTAAATGAACATTTGGGACTCCTGGGACCATATATAAGAGCAGAAGTTGAAGATAATATCATGGTAACTTTCAGGAATCAGGCCTCTAGACCCTATTCCTTCTATTCTAGCCTTATTTCTTATGAAG |
| | AAGATCAGAGGCAAGGAGCAGAACCTAGAAAAAACTTTGTCAAGCCTAATGAAACTAAAACTTACTTTTGGAAAGTGCAACATCATATGGCTCCCACTAAAGATGAGTTTGATTGCAAAGCCTGGGCATATTTCTCAGATGTTGA |
| | CCTAGAGAAAGATGTACACTCAGGACTGATTGGACCTCTTCTGGTCTGCCACACTAACACACTGAACCCTGCTCATGGTAGACAAGTGACTGTACAGGAATTTGCCCTGTTTTTCACCATCTTTGATGAGACAAAAAGCTGGTAC |
| | TTCACAGAAAATATGGAAAGAAACTGCAGGGCTCCCTGCAATATCCAGATGGAAGATCCTACCTTTAAAGAGAATTATAGGTTCCATGCAATCAATGGCTACATAATGGATACACTACCTGGGTTAGTAATGGCTCAGGATCAAA |
| | GGATTAGGTGGTATCTGCTCAGCATGGGCTCCAATGAGAACATCCATTCCATTCATTTCTCAGGACATGTGTTCACAGTTAGGAAAAAAGAGGAGTATAAAATGGCACTGTACAATCTCTATCCAGGTGTTTTTGAAACAGTAGA |
| | AATGTTACCCAGTAAAGCTGGAATTTGGAGGGTGGAGTGCCTTATAGGTGAGCATCTACATGCAGGAATGAGCACACTTTTTCTGGTGTACAGCAATAAGTGTCAGACACCCCTGGGAATGGCCTCAGGACACATTAGGGACTTT |
| | CAGATTACAGCCAGTGGACAATATGGTCAGTGGGCCCCAAAGCTGGCCAGACTTCATTATAGTGGTTCAATCAATGCTTGGAGCACTAAGGAGCCCTTTTCTTGGATCAAGGTGGATCTGCTGGCTCCCATGATTATTCATGGCA |
| | TCAAGACACAGGGTGCTAGGCAGAAGTTCAGCAGTCTCTACATCTCTCAGTTTATCATCATGTATTCACTTGATGGGAAGAAGTGGCAGACTTATAGAGGAAATTCCACAGGAACCTTAATGGTCTTCTTTGGAAATGTGGATTC |
| | ATCTGGAATAAAACACAATATTTTTAACCCTCCAATTATTGCTAGATACATCAGGTTGCACCCAACTCATTATAGCATTAGAAGCACTCTTAGAATGGAGTTGATGGGCTGTGATTTAAATAGTTGCTCCATGCCCTTGGGAATG |
| | GAGAGTAAAGCAATATCAGATGCCCAGATTACAGCTTCATCATACTTTACCAATATGTTTGCAACATGGTCTCCATCAAAAGCAAGACTTCACCTCCAAGGGAGAAGTAATGCCTGGAGACCTCAGGTGAATAATCCAAAAGAGT |
| | GGCTGCAAGTGGACTTCCAGAAGACTATGAAAGTCACAGGAGTAACAACTCAGGGAGTTAAATCTCTGCTTACCAGCATGTATGTAAAGGAGTTCCTCATCTCCAGCAGTCAAGATGGGCATCAGTGGACTCTCTTTTTTCAGAA |
| | TGGAAAAGTAAAGGTTTTTCAGGGAAATCAAGACTCCTTCACACCAGTGGTTAACAGTCTGGACCCTCCCTTACTGACTAGATACCTTAGGATTCACCCTCAGAGTTGGGTGCACCAGATTGCACTGAGAATGGAGGTTCTGGGC |
| | TGTGAGGCACAGGACCTCTACTGA |
| 18 | FVIII-QQ18 |
| | ATGCAAATAGAGCTCTCCACCTGCTTCTTTCTGTGCCTTTTGAGGTTCTGCTTTAGTGCAACAAGAAGATACTACCTGGGTGCAGTAGAACTGTCATGGGACTATATGCAATCTGATTTAGGGGAGCTGCCTGTGGATGCAAGAT |
| | TTCCACCCAGGGTGCCTAAAAGCTTTCCCTTCAACACTTCAGTTGTGTACAAAAAGACCCTGTTTGTAGAATTCACTGATCACCTTTTCAACATTGCTAAGCCAAGGCCACCCTGGATGGGTCTGCTTGGTCCTACCATCCAGGC |
| | AGAAGTTTATGATACTGTGGTCATTACACTTAAGAACATGGCTTCCCATCCAGTGAGTCTTCATGCTGTGGGTGTATCCTACTGGAAAGCTTCTGAGGGAGCTGAATATGATGACCAGACCAGTCAAAGGGAGAAAGAAGATGAC |
| | AAAGTCTTCCCAGGTGGAAGCCATACCTATGTGTGGCAGGTCCTGAAAGAGAATGGTCCAATGGCCTCTGATCCTCTGTGCCTTACCTACTCCTATCTTTCACATGTTGACCTGGTAAAAGACTTGAATTCAGGCCTCATTGGGG |
| | CCCTACTAGTTTGTAGAGAAGGGTCTCTGGCTAAGGAAAAGACTCAGACATTGCACAAATTTATACTACTTTTTGCTGTATTTGATGAGGGGAAAAGTTGGCACTCAGAGACAAAGAACTCCTTGATGCAGGATAGAGATGCTGC |
| | TTCAGCTAGAGCATGGCCTAAAATGCACACTGTCAATGGATATGTAAACAGAAGCCTGCCAGGTCTGATTGGTTGCCACAGGAAATCTGTCTATTGGCATGTGATTGGCATGGGCACCACTCCAGAAGTGCACTCAATATTCTTA |
| | GAGGGTCACACATTTCTTGTAAGAAACCATAGACAGGCTTCCTTGGAAATCTCACCTATAACCTTCCTTACAGCTCAAACCCTCTTGATGGACCTGGGACAGTTTCTACTGTTTTGTCATATCTCTTCCCACCAACATGATGGAA |
| | TGGAAGCTTATGTCAAAGTAGACAGCTGTCCAGAGGAACCCCAACTACAGATGAAAAATAATGAAGAAGCTGAAGACTATGATGATGATCTTACTGATAGTGAAATGGATGTAGTCAGGTTTGATGATGACAACTCTCCTTCCTT |
| | TATCCAAATTAGGTCAGTTGCTAAGAAGCATCCTAAAACTTGGGTACATTACATTGCAGCAGAAGAGGAGGACTGGGACTATGCCCCATTAGTCCTAGCCCCTGATGATAGAAGTTATAAAAGTCAATATTTGAACAATGGACCA |
| | CAGAGAATTGGTAGGAAGTACAAAAAAGTCAGATTTATGGCATACACTGATGAGACCTTTAAGACAAGGGAAGCTATTCAGCATGAATCTGGAATCTTGGGACCTTTACTTTATGGAGAAGTTGGGGACACACTGTTGATTATAT |
| | TTAAGAATCAAGCAAGCAGACCATATAACATCTACCCCCATGGAATCACTGATGTCAGGCCCTTGTATTCAAGAAGGTTACCTAAAGGTGTAAAACATTTGAAGGATTTTCCTATTCTGCCAGGTGAAATATTCAAATATAAATG |
| | GACAGTGACTGTAGAAGATGGCCCAACTAAAAGTGATCCCAGGTGCCTGACCAGATATTACTCTAGTTTTGTCAATATGGAGAGAGATCTAGCTTCAGGACTCATTGGTCCTCTCCTCATCTGCTACAAAGAATCAGTAGATCAA |
| | CAGGGAAACCAGATAATGAGTGATAAGAGGAATGTCATCCTGTTTAGTGTATTTGATGAAAACAGAAGCTGGTACCTCACAGAAAATATACAAAGATTTCTCCCCAATCCAGCTGGGGTGCAGTTGGAGGATCCAGAATTCCAAG |
| | CAAGTAACATCATGCACAGCATCAATGGGTATGTATTTGACTCTTTGCAGTTGTCAGTATGTTTGCATGAAGTGGCATACTGGTACATTCTATCAATTGGGGCCCAGACTGATTTCCTTAGTGTCTTCTTCTCTGGTTATACATT |
| | CAAACACAAAATGGTCTATGAAGACACCCTCACCCTATTCCCATTCTCAGGGGAAACTGTCTTCATGAGTATGGAAAACCCAGGTCTATGGATTTTGGGGTGCCACAACTCAGACTTTAGGAACAGAGGCATGACTGCATTACTG |
| | AAGGTTTCAAGTTGTGATAAGAACACTGGTGATTATTATGAAGACAGTTATGAGGATATTTCAGCATACTTGCTGAGTAAAAACAATGCAATAGAACCAAGAAGCTTCTCTCAAAACCCACCAGTCTTGAAAAGACATCAAAGAG |
| | AAATAACCAGGACTACTCTTCAGTCTGATCAAGAGGAAATTGATTATGATGACACCATATCAGTTGAAATGAAGAAGGAAGATTTTGATATTTATGATGAGGATGAAAATCAGAGCCCAAGGAGCTTTCAAAAGAAAACCAGACA |
| | CTATTTTATTGCAGCAGTAGAGAGACTCTGGGATTATGGGATGAGTTCATCCCCCCATGTTCTAAGAAACAGGGCACAGAGTGGCAGTGTCCCACAGTTCAAGAAAGTAGTTTTCCAGGAATTTACAGATGGTTCCTTTACTCAG |
| | CCATTATACAGGGGAGAACTAAATGAACATTTGGGACTCTTAGGGCCATATATAAGAGCTGAAGTTGAAGATAATATCATGGTAACTTTCAGGAATCAGGCCTCTAGACCATATTCCTTCTATAGTAGCCTTATTTCTTATGAGG |
| | AAGATCAGAGGCAAGGAGCAGAGCCTAGGAAAAACTTTGTTAAGCCTAATGAAACAAAAACTTACTTTTGGAAAGTGCAACATCATATGGCACCCACCAAAGATGAGTTTGACTGCAAAGCCTGGGCCTATTTCTCTGATGTTGA |
| | CTTAGAAAAAGATGTGCACTCTGGGCTGATTGGACCACTTCTGGTCTGCCACACTAACACACTGAACCCAGCACATGGCAGACAAGTGACTGTCCAGGAATTTGCTCTGTTTTTCACCATCTTTGATGAGACAAAAAGCTGGTAC |
| | TTCACTGAAAATATGGAAAGGAACTGCAGGGCTCCCTGCAATATCCAGATGGAAGATCCAACCTTTAAAGAGAATTATAGATTCCATGCCATCAATGGGTACATAATGGATACACTACCTGGCTTAGTAATGGCTCAGGATCAAA |
| | GGATTAGGTGGTATCTGCTCAGCATGGGCTCCAATGAAAACATCCATAGCATTCATTTCAGTGGACATGTTTTCACTGTAAGGAAAAAAGAGGAGTATAAAATGGCACTGTACAATCTCTATCCAGGAGTTTTTGAGACAGTTGA |
| | AATGTTACCATCTAAAGCAGGAATTTGGAGGGTTGAATGCCTTATTGGGGAACATCTACATGCAGGTATGAGCACACTTTTTCTGGTGTACAGCAATAAGTGTCAGACTCCACTGGGAATGGCATCTGGCCACATTAGAGATTTT |
| | CAGATTACTGCATCAGGGCAATATGGACAGTGGGCCCCAAAGCTGGCCAGACTTCATTATAGTGGATCAATCAATGCTTGGAGCACCAAGGAGCCCTTTTCTTGGATCAAGGTGGATCTGCTTGCACCCATGATTATTCATGGGA |
| | TCAAGACCCAGGGTGCCAGACAGAAGTTCAGCAGCCTCTACATCTCTCAGTTTATCATCATGTATAGCCTTGATGGAAAGAAGTGGCAGACTTATAGGGGAAATAGCACAGGAACCTTAATGGTCTTCTTTGGTAATGTTGATTC |
| | AAGTGGGATAAAACACAATATTTTTAACCCTCCAATTATAGCTAGATACATCAGATTGCACCCCACTCATTATAGCATTAGGAGCACTCTTAGGATGGAGTTGATGGGCTGTGATTTAAATAGTTGCAGTATGCCCTTGGGAATG |
| | GAGAGTAAAGCAATATCAGATGCTCAGATTACTGCATCAAGCTACTTTACTAATATGTTTGCTACATGGTCTCCATCAAAAGCTAGACTTCACCTCCAAGGGAGATCCAATGCTTGGAGACCTCAGGTGAATAATCCAAAAGAGT |
| | GGCTGCAAGTGGACTTCCAGAAGACTATGAAAGTCACAGGGGTAACAACCCAGGGAGTAAAATCTCTGCTTACCAGCATGTATGTCAAGGAGTTCCTCATCTCCAGCTCACAAGATGGGCATCAGTGGACTCTCTTTTTTCAGAA |
| | TGGGAAAGTAAAGGTTTTTCAGGGAAATCAAGACTCCTTCACACCAGTGGTGAACTCTTTGGACCCACCTTTACTGACTAGATACCTTAGGATTCACCCACAGAGTTGGGTGCACCAGATAGCTCTGAGAATGGAGGTTCTGGGC |
| | TGTGAGGCCCAGGACCTCTACTGA |
| 19 | Human FVIII 3550; 5810 polypeptide sequence including signal peptide (total 1457 a.a.s) |
| | MQIELSTCFFLCLLRFCFSATRRYYLGAVELSWDYMQSDLGELPVDARFPPRVPKSFPENTSVVYKKTLFVEFTDHLENIAKPRPPWMGLLGPTIQAEVYDTVVITLKNMASHPVSLHAVGVSYWKASEGAEYDDQTSQREKEDD |
| | KVFPGGSHTYVWQVLKENGPMASDPLCLTYSYLSHVDLVKDLNSGLIGALLVCREGSLAKEKTQTLHKFILLFAVEDEGKSWHSETKNSLMQDRDAASARAWPKMHTVNGYVNRSLPGLIGCHRKSVYWHVIGMGTTPEVHSIFL |
| | EGHTFLVRNHRQASLEISPITFLTAQTLLMDLGQFLLFCHISSHQHDGMEAYVKVDSCPEEPQLQMKNNEEAEDYDDDLTDSEMDVVRFDDDNSPSFIQIRSVAKKHPKTWVHYIAAEEEDWDYAPLVLAPDDRSYKSQYLNNGP |
| | QRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQ |
| | QGNQIMSDKRNVILFSVFDENRSWYLTENIQRFLPNPAGVQLEDPEFQASNIMHSINGYVFDSLQLSVCLHEVAYWYILSIGAQTDFLSVFFSGYTEKHKMVYEDTLTLFPESGETVEMSMENPGLWILGCHNSDERNRGMTALL |
| | KVSSCDKNTGDYYEDSYEDISAYLLSKNNAIEPRSFSQNPPVLKRHQREITRTTLQSDQEEIDYDDTISVEMKKEDEDIYDEDENQSPRSFQKKTRHYFIAAVERLWDYGMSSSPHVLRNRAQSGSVPQFKKVVFQEFTDGSFTQ |
| | PLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWY |
| | FTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDE |
| | QITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIENPPIIARYIRLHPTHYSIRSTLRMELMGCDLNSCSMPLGM |
| | ESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLG |
| | CEAQDLY |
| 20 | N-terminal Signal peptide of WT human FVIII |
| | MQIELSTCFFLCLLRFCFS |
| 21 | Expression plasmid with exemplary codon optimized FVIII nucleic acid (QQ8) |
| | TCTAGAGCTAGCATATGGATCCATCGATTTAGGGATAACAGGGTAATTATCAGCACACAATTGCCCATTATACGCGCGTATAATGGACTATTGTGTGCTGATATCTGTACACTTAAGGGCTAGATCTTAGCTTACGTCACTAGAG |
| | GGTCCACGTTTAGTTTTTAAGATCCATTGATCTCCTAAACGCTGCAAGATTCGCAACCTGGTATACTTAGCCTAGGCGCTAGGTCCTAGTGCAGCGGGACTTTTTTTCTAAAGTCGTTGAGAGGAGGAGTCGTCAGACCAGATAG |
| | CTTTGATGTCCTGATCGGAAGGATCGTTGGCCCCCCTGCAGGCAGCTGTTAATTAACTGCGCGCTCGCTCGCTCACTGAGGCCGCCCGGGCAAAGCCCGGGCGTCGGGCGACCTTTGGTCGCCCGGCCTCAGTGAGCGAGCGAGC |
| | GCGCAGAGAGGGAGTGGCCAACTCCATCACTAGGGGTTCCTGTTTGCTGCTTGCAATGTTTGCCCATTTTAGGGTGGACACAGGACGCTGTGGTTTCTGAGCCAGGGGGCGACTCAGATCCCAGCCAGTGGACTTAGCCCCTGTT |
| | TGCTCCTCCGATAACTGGGGTGACCTTGGTTAATATTCACCAGCAGCCTCCCCCGTTGCCCCTCTGGATCCACTGCTTAAATACGGACGAGGACAGGGCCCTGTCTCCTCAGCTTCAGGCACCACCACTGACCTGGGACAGTGAA |
| | TCGCCACCATGCAGATTGAGCTGAGCACCTGCTTCTTCCTGTGCCTGCTGAGGTTCTGCTTCTCTGCCACCAGGAGATACTACCTGGGGGCTGTGGAGCTGAGCTGGGACTACATGCAGTCTGACCTGGGGGAGCTGCCTGTGGA |
| | TGCCAGGTTCCCCCCCAGAGTGCCCAAGAGCTTCCCCTTCAACACCTCTGTGGTGTACAAGAAGACCCTGTTTGTGGAGTTCACTGACCACCTGTTCAACATTGCCAAGCCCAGGCCCCCCTGGATGGGCCTGCTGGGCCCCACC |
| | ATCCAGGCTGAGGTGTATGACACTGTGGTGATCACCCTGAAGAACATGGCCAGCCACCCTGTGAGCCTGCATGCTGTGGGGGTGAGCTACTGGAAGGCCTCTGAGGGGGCTGAGTATGATGACCAGACCAGCCAGAGGGAGAAGG |
| | AGGATGACAAGGTGTTCCCTGGGGGCAGCCACACCTATGTGTGGCAGGTGCTGAAGGAGAATGGCCCCATGGCCTCTGACCCCCTGTGCCTGACCTACAGCTACCTGAGCCATGTGGACCTGGTGAAGGACCTGAACTCTGGCCT |
| | GATTGGGGCCCTGCTGGTGTGCAGGGAGGGCAGCCTGGCCAAGGAGAAGACCCAGACCCTGCACAAGTTCATCCTGCTGTTTGCTGTGTTTGATGAGGGCAAGAGCTGGCACTCTGAAACCAAGAACAGCCTGATGCAGGACAGG |
| | GATGCTGCCTCTGCCAGGGCCTGGCCCAAGATGCACACTGTGAATGGCTATGTGAACAGGAGCCTGCCTGGCCTGATTGGCTGCCACAGGAAGTCTGTGTACTGGCATGTGATTGGCATGGGCACCACCCCTGAGGTGCACAGCA |
| | TCTTCCTGGAGGGCCACACCTTCCTGGTCAGGAACCACAGGCAGGCCAGCCTGGAGATCAGCCCCATCACCTTCCTGACTGCCCAGACCCTGCTGATGGACCTGGGCCAGTTCCTGCTGTTCTGCCACATCAGCAGCCACCAGCA |
| | TGATGGCATGGAGGCCTATGTGAAGGTGGACAGCTGCCCTGAGGAGCCCCAGCTGCAGATGAAGAACAATGAGGAGGCTGAGGACTATGATGATGACCTGACTGACTCTGAGATGGATGTGGTGAGGTTTGATGATGACAACAGC |
| | CCCAGCTTCATCCAGATCAGGTCTGTGGCCAAGAAGCACCCCAAGACCTGGGTGCACTACATTGCTGCTGAGGAGGAGGACTGGGACTATGCCCCCCTGGTGCTGGCCCCTGATGACAGGAGCTACAAGAGCCAGTACCTGAACA |
| | ATGGCCCCCAGAGGATTGGCAGGAAGTACAAGAAGGTCAGGTTCATGGCCTACACTGATGAAACCTTCAAGACCAGGGAGGCCATCCAGCATGAGTCTGGCATCCTGGGCCCCCTGCTGTATGGGGAGGTGGGGGACACCCTGCT |
| | GATCATCTTCAAGAACCAGGCCAGCAGGCCCTACAACATCTACCCCCATGGCATCACTGATGTGAGGCCCCTGTACAGCAGGAGGCTGCCCAAGGGGGTGAAGCACCTGAAGGACTTCCCCATCCTGCCTGGGGAGATCTTCAAG |
| | TACAAGTGGACTGTGACTGTGGAGGATGGCCCCACCAAGTCTGACCCCAGGTGCCTGACCAGATACTACAGCAGCTTTGTGAACATGGAGAGGGACCTGGCCTCTGGCCTGATTGGCCCCCTGCTGATCTGCTACAAGGAGTCTG |
| | TGGACCAGCAGGGCAACCAGATCATGTCTGACAAGAGGAATGTGATCCTGTTCTCTGTGTTTGATGAGAACAGGAGCTGGTACCTGACTGAGAACATCCAGAGGTTCCTGCCCAACCCTGCTGGGGTGCAGCTGGAGGACCCTGA |
| | GTTCCAGGCCAGCAACATCATGCACAGCATCAATGGCTATGTGTTTGACAGCCTGCAGCTGTCTGTGTGCCTGCATGAGGTGGCCTACTGGTACATCCTGAGCATTGGGGCCCAGACTGACTTCCTGTCTGTGTTCTTCTCTGGC |
| | TACACCTTCAAGCACAAGATGGTGTATGAGGACACCCTGACCCTGTTCCCCTTCTCTGGGGAGACTGTGTTCATGAGCATGGAGAACCCTGGCCTGTGGATTCTGGGCTGCCACAACTCTGACTTCAGGAACAGGGGCATGACTG |
| | CCCTGCTGAAAGTCTCCAGCTGTGACAAGAACACTGGGGACTACTATGAGGACAGCTATGAGGACATCTCTGCCTACCTGCTGAGCAAGAACAATGCCATTGAGCCCAGGAGCTTCAGCCAGAACCCCCCAGTGCTGAAGAGGCA |
| | CCAGAGGGAGATCACCAGGACCACCCTGCAGTCTGACCAGGAGGAGATTGACTATGATGACACCATCTCTGTGGAGATGAAGAAGGAGGACTTTGACATCTACGACGAGGACGAGAACCAGAGCCCCAGGAGCTTCCAGAAGAAG |
| | ACCAGGCACTACTTCATTGCTGCTGTGGAGAGGCTGTGGGACTATGGCATGAGCAGCAGCCCCCATGTGCTGAGGAACAGGGCCCAGTCTGGCTCTGTGCCCCAGTTCAAGAAGGTGGTGTTCCAGGAGTTCACTGATGGCAGCT |
| | TCACCCAGCCCCTGTACAGAGGGGAGCTGAATGAGCACCTGGGCCTGCTGGGCCCCTACATCAGGGCTGAGGTGGAGGACAACATCATGGTGACCTTCAGGAACCAGGCCAGCAGGCCCTACAGCTTCTACAGCAGCCTGATCAG |
| | CTATGAGGAGGACCAGAGGCAGGGGGCTGAGCCCAGGAAGAACTTTGTGAAGCCCAATGAAACCAAGACCTACTTCTGGAAGGTGCAGCACCACATGGCCCCCACCAAGGATGAGTTTGACTGCAAGGCCTGGGCCTACTTCTCT |
| | GATGTGGACCTGGAGAAGGATGTGCACTCTGGCCTGATTGGCCCCCTGCTGGTGTGCCACACCAACACCCTGAACCCTGCCCATGGCAGGCAGGTGACTGTGCAGGAGTTTGCCCTGTTCTTCACCATCTTTGATGAAACCAAGA |
| | GCTGGTACTTCACTGAGAACATGGAGAGGAACTGCAGGGCCCCCTGCAACATCCAGATGGAGGACCCCACCTTCAAGGAGAACTACAGGTTCCATGCCATCAATGGCTACATCATGGACACCCTGCCTGGCCTGGTGATGGCCCA |
| | GGACCAGAGGATCAGGTGGTACCTGCTGAGCATGGGCAGCAATGAGAACATCCACAGCATCCACTTCTCTGGCCATGTGTTCACTGTGAGGAAGAAGGAGGAGTACAAGATGGCCCTGTACAACCTGTACCCTGGGGTGTTTGAG |
| | ACTGTGGAGATGCTGCCCAGCAAGGCTGGCATCTGGAGGGTGGAGTGCCTGATTGGGGAGCACCTGCATGCTGGCATGAGCACCCTGTTCCTGGTGTACAGCAACAAGTGCCAGACCCCCCTGGGCATGGCCTCTGGCCACATCA |
| | GGGACTTCCAGATCACTGCCTCTGGCCAGTATGGCCAGTGGGCCCCCAAGCTGGCCAGGCTGCACTACTCTGGCAGCATCAATGCCTGGAGCACCAAGGAGCCCTTCAGCTGGATCAAGGTGGACCTGCTGGCCCCCATGATCAT |
| | CCATGGCATCAAGACCCAGGGGGCCAGGCAGAAGTTCAGCAGCCTGTACATCAGCCAGTTCATCATCATGTACAGCCTGGATGGCAAGAAGTGGCAGACCTACAGGGGCAACAGCACTGGCACCCTGATGGTGTTCTTTGGCAAT |
| | GTGGACAGCTCTGGCATCAAGCACAACATCTTCAACCCCCCCATCATTGCCAGATACATCAGGCTGCACCCCACCCACTACAGCATCAGGAGCACCCTGAGGATGGAGCTGATGGGCTGTGACCTGAACAGCTGCAGCATGCCCC |
| | TGGGCATGGAGAGCAAGGCCATCTCTGATGCCCAGATCACTGCCAGCAGCTACTTCACCAACATGTTTGCCACCTGGAGCCCCAGCAAGGCCAGGCTGCACCTGCAGGGCAGGAGCAATGCCTGGAGGCCCCAGGTCAACAACCC |
| | CAAGGAGTGGCTGCAGGTGGACTTCCAGAAGACCATGAAGGTGACTGGGGTGACCACCCAGGGGGTGAAGAGCCTGCTGACCAGCATGTATGTGAAGGAGTTCCTGATCAGCAGCAGCCAGGATGGCCACCAGTGGACCCTGTTC |
| | TTCCAGAATGGCAAGGTGAAGGTGTTCCAGGGCAACCAGGACAGCTTCACCCCTGTGGTGAACAGCCTGGACCCCCCCCTGCTGACCAGATACCTGAGGATTCACCCCCAGAGCTGGGTGCACCAGATTGCCCTGAGGATGGAGG |
| | TGCTGGGCTGTGAGGCCCAGGACCTGTACTGAAATAAAAGATCTTTATTTTCATTAGATCTGTGTGTTGGTTTTTTGTGTGAGGAACCCCTAGTGATGGAGTTGGCCACTCCCTCTCTGCGCGCTCGCTCGCTCACTGAGGCCGG |
| | GCGACCAAAGGTCGCCCGACGCCCGGGCTTTGCCCGGGCGGCCTCAGTGAGCGAGCGAGCGCGCAGTTAATTAAGGCGCCCTAGGCCGACCCTTAGACTCTGTACTCAGTTCTATAAACGAGCCATTGGATACGAGATCCGTAGA |
| | TTGATAAGGGACACGGAATATCCCCGGACGCAATAGACACCGGTGGACAGCTTGGTATCCTGAGCACAGTCGCGCGTCCGAATCTAGCTCTACTTTAGAGGCCCCGGATTCTGATGGTCGTAGACCGCAGAACCGATTGGGGGGA |
| | TGAGATCTACTAGTTATCAGCACACAATTGCCCATTATACGCGCGTATAATGGACTATTGTGTGCTGATATAGGGATAACAGGGTAATTCTAGAGCTAGCATATGGATCCATCGATTTGATGCGGTATTTTCTCCTTACGCATCT |
| | GTGCGGTATTTCACACCGCATACGTCAAAGCAACCATAGTACGCGCCCTGTAGCGGCGCATTAAGCGCGGCGGGTGTGGTGGTTACGCGCAGCGTGACCGCTACACTTGCCAGCGCCCTAGCGCCCGCTCCTTTCGCTTTCTTCC |
| | CTTCCTTTCTCGCCACGTTCGCCGGCTTTCCCCGTCAAGCTCTAAATCGGGGGCTCCCTTTAGGGTTCCGATTTAGTGCTTTACGGCACCTCGACCCCAAAAAACTTGATTTGGGTGATGGTTCACGTAGTGGGCCATCGCCCTG |
| | ATAGACGGTTTTTCGCCCTTTGACGTTGGAGTCCACGTTCTTTAATAGTGGACTCTTGTTCCAAACTGGAACAACACTCAACTCTATCTCGGGCTATTCTTTTGATTTATAAGGGATTTTGCCGATTTCGGTCTATTGGTTAAAA |
| | AATGAGCTGATTTAACAAAAATTTAACGCGAATTTTAACAAAATATTAACGTTTACAATTTTATGGTGCACTCTCAGTACAATCTGCTCTGATGCCGCATAGTTAAGCCAGCCCCGACACCCGCCAACACCCGCTGACGCGCCCT |
| | GACGGGCTTGTCTGCTCCCGGCATCCGCTTACAGACAAGCTGTGACCGTCTCCGGGAGCTGCATGTGTCAGAGGTTTTCACCGTCATCACCGAAACGCGCGAGACGAAAGGGCCTCGTGATACGCCTATTTTTATAGGTTAATGT |
| | CATGATAATAATGGTTTCTTAGACGTCAGGTGGCACTTTTCGGGGAAATGTGCGCGGAACCCCTATTTGTTTATTTTTCTAAATACATTCAAATATGTATCCGCTCATGAGACAATAACCCTGATAAATGCTTCAATAATATTGA |
| | AAAAGGAAGAGTATGAGCCATATTCAACGGGAAACGTCGAGGCCGCGATTAAATTCCAACATGGATGCTGATTTATATGGGTATAAATGGGCTCGCGATAATGTCGGGCAATCAGGTGCGACAATCTATCGCTTGTATGGGAAGC |
| | CCGATGCGCCAGAGTTGTTTCTGAAACATGGCAAAGGTAGCGTTGCCAATGATGTTACAGATGAGATGGTCAGACTAAACTGGCTGACGGAATTTATGCCTCTTCCGACCATCAAGCATTTTATCCGTACTCCTGATGATGCATG |
| | GTTACTCACCACTGCGATCCCCGGAAAAACAGCATTCCAGGTATTAGAAGAATATCCTGATTCAGGTGAAAATATTGTTGATGCGCTGGCAGTGTTCCTGCGCCGGTTGCATTCGATTCCTGTTTGTAATTGTCCTTTTAACAGC |
| | GATCGCGTATTTCGTCTCGCTCAGGCGCAATCACGAATGAATAACGGTTTGGTTGATGCGAGTGATTTTGATGACGAGCGTAATGGCTGGCCTGTTGAACAAGTCTGGAAAGAAATGCATAAACTTTTGCCATTCTCACCGGATT |
| | CAGTCGTCACTCATGGTGATTTCTCACTTGATAACCTTATTTTTGACGAGGGGAAATTAATAGGTTGTATTGATGTTGGACGAGTCGGAATCGCAGACCGATACCAGGATCTTGCCATCCTATGGAACTGCCTCGGTGAGTTTTC |
| | TCCTTCATTACAGAAACGGCTTTTTCAAAAATATGGTATTGATAATCCTGATATGAATAAATTGCAGTTTCATTTGATGCTCGATGAGTTTTTCTAAGCGTATAATGGTCTAGAGCTAGCATATGGATCCATCGATTCCATTATA |
| | CGCCTGTCAGACCAAGTTTACTCATATATACTTTAGATTGATTTAAAACTTCATTTTTAATTTAAAAGGATCTAGGTGAAGATCCTTTTTGATAATCTCATGACCAAAATCCCTTAACGTGAGTTTTCGTTCCACTGAGCGTCAG |
| | ACCCCGTAGAAAAGATCAAAGGATCTTCTTGAGATCCTTTTTTTCTGCGCGTAATCTGCTGCTTGCAAACAAAAAAACCACCGCTACCAGCGGTGGTTTGTTTGCCGGATCAAGAGCTACCAACTCTTTTTCCGAAGGTAACTGG |
| | CTTCAGCAGAGCGCAGATACCAAATACTGTTCTTCTAGTGTAGCCGTAGTTAGGCCACCACTTCAAGAACTCTGTAGCACCGCCTACATACCTCGCTCTGCTAATCCTGTTACCAGTGGCTGCTGCCAGTGGCGATAAGTCGTGT |
| | CTTACCGGGTTGGACTCAAGACGATAGTTACCGGATAAGGCGCAGCGGTCGGGCTGAACGGGGGGTTCGTGCACACAGCCCAGCTTGGAGCGAACGACCTACACCGAACTGAGATACCTACAGCGTGAGCTATGAGAAAGCGCCA |
| | CGCTTCCCGAAGGGAGAAAGGCGGACAGGTATCCGGTAAGCGGCAGGGTCGGAACAGGAGAGCGCACGAGGGAGCTTCCAGGGGGAAACGCCTGGTATCTTTATAGTCCTGTCGGGTTTCGCCACCTCTGACTTGAGCGTCGATT |
| | TTTGTGATGCTCGTCAGGGGGGCGGAGCCTATGGAAAAACGCCAGCAACGCGGCCTTTTTACGGTTCCTGGCCTTTTGCTGGCCTTTTGCTCACATGT |
| 22 | |
| 23 | SFSQNPPVLKRHOR |
| 24 | |
| | Isoform A human FVIII |
| | MQIELSTCFF LCLLRFCFSA TRRYYLGAVE LSWDYMQSDL GELPVDARFPPRVPKSFPEN TSVVYKKTLF VEFTDHLENI AKPRPPWMGL LGPTIQAEVYDTVVITLKNM ASHPVSLHAV GVSYWKASEG |
| | AEYDDQTSQR EKEDDKVFPG GSHTYVWQVL KENGPMASDP LCLTYSYLSH VDLVKDLNSG LIGALLVCRE GSLAKEKTQT LHKFILLEAV FDEGKSWHSE TKNSLMQDRD AASARAWPKM HTVNGYVNRS |
| | LPGLIGCHRK SVYWHVIGMG TTPEVHSIFL EGHTFLVRNH RQASLEISPI TFLTAQTLLM DLGQFLLFCH ISSHQHDGME AYVKVDSCPE EPQLRMKNNE EAEDYDDDLT DSEMDVVRED DDNSPSFIQI |
| | RSVAKKHPKT WVHYIAAEEE DWDYAPLVLA PDDRSYKSQY LNNGPQRIGR KYKKVRFMAY TDETEKTREA IQHESGILGP LLYGEVGDTL LIIFKNQASR PYNIYPHGIT DVRPLYSRRL PKGVKHLKDE |
| | PILPGEIFKY KWTVTVEDGP TKSDPRCLTR YYSSFVNMER DLASGLIGPL LICYKESVDQ RGNQIMSDKR NVILESVEDE NRSWYLTENI QRFLPNPAGV QLEDPEFQAS NIMHSINGYV FDSLQLSVCL |
| | HEVAYWYILS IGAQTDFLSV FFSGYTFKHK MVYEDTLTLF PFSGETVEMS MENPGLWILG CHNSDERNRG MTALLKVSSC DKNTGDYYED SYEDISAYLL SKNNAIEPRS FSQNSRHPST RQKQFNATTI |
| | PENDIEKTDP WFAHRTPMPK IQNVSSSDLL MLLRQSPTPH GLSLSDLQEA KYETFSDDPS PGAIDSNNSL SEMTHFRPQL HHSGDMVFTP ESGLQLRLNE KLGTTAATEL KKLDEKVSST SNNLISTIPS |
| | DNLAAGTDNT SSLGPPSMPV HYDSQLDTTL FGKKSSPLTE SGGPLSLSEE NNDSKLLESG LMNSQESSWG KNVSSTESGR LEKGKRAHGP ALLTKDNALF KVSISLLKTN KTSNNSATNR KTHIDGPSLL |
| | IENSPSVWQN ILESDTEFKK VTPLIHDRML MDKNATALRL NHMSNKTTSS KNMEMVQQKK EGPIPPDAQN PDMSFFKMLF LPESARWIQR THGKNSLNSG QGPSPKQLVS LGPEKSVEGQ NELSEKNKVV |
| | VGKGEFTKDV GLKEMVFPSS RNLFLTNLDN LHENNTHNQE KKIQEEIEKK ETLIQENVVL PQIHTVTGTK NEMKNLELLS TRQNVEGSYD GAYAPVLQDF RSLNDSTNRT KKHTAHFSKK GEEENLEGLG |
| | NQTKQIVEKY ACTTRISPNT SQQNFVTQRS KRALKQFRLP LEETELEKRI IVDDTSTQWS KNMKHLTPST LTQIDYNEKE KGAITQSPLS DCLTRSHSIP QANRSPLPIA KVSSFPSIRP IYLTRVLFQD |
| | NSSHLPAASY RKKDSGVQES SHFLQGAKKN NLSLAILTLE MTGDQREVGS LGTSATNSVT YKKVENTVLP KPDLPKTSGK VELLPKVHIY QKDLEPTETS NGSPGHLDLV EGSLLQGTEG AIKWNEANRP |
| | GKVPFLRVAT ESSAKTPSKL LDPLAWDNHY GTQIPKEEWK SQEKSPEKTA FKKKDTILSL NACESNHAIA AINEGQNKPE IEVTWAKQGR TERLCSQNPP VLKRHQREIT RTTLQSDQEE IDYDDTISVE |
| | MKKEDEDIYD EDENQSPRSF QKKTRHYFIA AVERLWDYGM SSSPHVLRNR AQSGSVPQFK KVVFQEFTDG SFTQPLYRGE LNEHLGLLGP YIRAEVEDNI MVTERNQASR PYSFYSSLIS YEEDQRQGAE |
| | PRKNFVKPNE TKTYFWKVQH HMAPTKDEFD CKAWAYFSDV DLEKDVHSGL IGPLLVCHTN TLNPAHGRQV TVQEFALFFT IFDETKSWYF TENMERNCRA PCNIQMEDPT FKENYRFHAI NGYIMDTLPG |
| | LVMAQDQRIR WYLLSMGSNE NIHSIHFSGH VFTVRKKEEY KMALYNLYPG VFETVEMLPS KAGIWRVECL IGEHLHAGMS TLFLVYSNKC QTPLGMASGH IRDFQITASG QYGQWAPKLA RLHYSGSINA |
| | WSTKEPFSWI KVDLLAPMII HGIKTQGARQ KFSSLYISQF IIMYSLDGKK WQTYRGNSTG TLMVFFGNVD SSGIKHNIEN PPIIARYIRL HPTHYSIRST LRMELMGCDL NSCSMPLGME SKAISDAQIT |
| | ASSYFTNMFA TWSPSKARLH LQGRSNAWRP QVNNPKEWLQ VDFQKTMKVT GVTTQGVKSL LTSMYVKEFL ISSSQDGHQW TLFFQNGKVK |
| | VFQGNQDSFT PVVNSLDPPL LTRYLRIHPQ SWVHQIALRM EVLGCEAQDL |
| | Y |
| 25 | Isoform B Human FVIII |
| | MRIQDPGKVFFGNVDSSGIKHNIFNPPIIARYIRLHPTHYSIRSTLRMELMGCDLNSCSMLGMESKAISDAQITASSYFTNMFATWSPSKARLHLQGRSNAWRPQVNNPKEWLQVDFQKMKVTGVTTQGVKSLLTSMYVKEFLIS |
| | SSQDGHQWTLFFQNGKVKVFQGNQDSFTPVVNSLDPPLLTRYLRIHPQSWVHQIALRMEVLGCEAQDLY |
| 26 | >ITR-to-ITR construct with FVIII-QQ1 nucleic acid |
| | >[L-ITR]-[ACTUS101-LSP]-[5′ UTR]-[FVIII-QQ1(w/sp)]-[3′ UTR]-[PolIII_terminator]-[hGHpolyA]-[PolII_terminator-rev][R-ITR] |
| | CTGCGCGCTCGCTCGCTCACTGAGGCCGCCCGGGCAAAGCCCGGGCGTCGGGCGACCTTTGGTCGCCCGGCCTCAGTGAGCGAGCGAGCGCGCAGAGAGGGAGTGGCCAACTCCATCACTAGGGGTTCCTTGTAGTTAATGATTA |
| | ACCCGCCATGCTACTTATCTACGGCGCGCCACGCGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACT |
| | AGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACTAGTTCTAGAGCGGCCGCCAGTGTGCTGGAATTC |
| | GGCTTTTTTAGGGCTGGAAGCTACCTTTGACATCATTTCCTCTGCGAATGCATGTATAATTTCTACAGAACCTATTAGAAAGGATCACCCAGCCTCTGCTTTTGTACAACTTTCCCTTAAAAAACTGCCAATCCCACTGCTGTTT |
| | GGCCCAATAGTGAGAACTTTTTCCTGCTGCCTCTTGGTGCTTTTGCCTATGGCCCCTATTCTGCCTGCTGAAGACACTCTTGCCAGCATGGACTTAAACCCCTCCAGCTCTGACAATCCTCTTTCTCTTTTGTTTTACATGAAGG |
| | GTCTGGCAGCCAAAGCAATCACTCAAAGTTCAAACCTTATCATTTTTTGCTTTGTTCCTCTTGGCCTTGGTTTTGTACATCAGCTTTGAAAATACCATCCCAGGGTTAATGCTGGGGTTAATTTATAACTGAGAGTGCTCTAGTT |
| | CTGCAATACAGGACATGCTATAAAAATGGAAAGATGTTGCTTTCTGAGAGATCAGGTGGATTCTTGGGCATTTGCTTAAGCTTGGTACCGCCACCATGCAGATTGAGCTGAGCACCTGCTTCTTCCTGTGCCTGCTGAGATTCTG |
| | CTTCTCTGCCACCAGAAGATACTACCTGGGTGCAGTGGAGCTGAGCTGGGACTACATGCAGAGTGACCTGGGTGAGCTGCCTGTTGATGCCAGATTCCCCCCCAGAGTGCCCAAGAGCTTCCCCTTCAACACCTCTGTTGTGTAC |
| | AAGAAGACCCTGTTTGTTGAGTTCACTGACCACCTGTTCAACATTGCCAAGCCCAGACCCCCCTGGATGGGCCTGCTGGGCCCCACCATCCAGGCAGAGGTGTATGATACTGTGGTGATCACCCTGAAGAACATGGCCAGCCACC |
| | CAGTTAGCCTGCATGCAGTGGGGGTCAGCTACTGGAAGGCCTCAGAAGGGGCTGAGTATGATGATCAGACCAGCCAGAGAGAGAAGGAGGATGACAAGGTGTTCCCAGGGGGTAGCCACACCTATGTATGGCAGGTGCTGAAGGA |
| | GAATGGCCCCATGGCCTCTGACCCCCTGTGCCTGACCTACAGCTACCTGAGCCATGTGGACCTGGTGAAGGACCTGAACAGTGGACTGATTGGTGCCCTGCTGGTGTGCAGAGAGGGCAGCCTGGCCAAGGAGAAGACCCAGACC |
| | CTGCACAAGTTCATCCTGCTGTTTGCTGTGTTTGATGAAGGCAAGAGCTGGCACAGTGAGACCAAGAACAGCCTGATGCAGGACAGAGATGCTGCATCAGCTAGAGCCTGGCCCAAGATGCACACTGTCAATGGTTATGTAAACA |
| | GAAGCCTGCCTGGCCTGATTGGCTGCCACAGAAAGTCAGTATACTGGCATGTTATAGGCATGGGCACCACCCCAGAGGTGCACAGCATCTTCCTGGAGGGCCACACCTTCCTGGTGAGAAACCACAGACAGGCCAGCCTGGAGAT |
| | CAGCCCCATCACCTTCCTGACAGCCCAGACCCTGCTGATGGACCTGGGCCAGTTCCTGCTGTTCTGCCACATCAGCAGCCACCAGCATGATGGGATGGAGGCCTATGTAAAGGTGGACAGCTGCCCAGAGGAGCCCCAGCTGCAG |
| | ATGAAGAACAATGAAGAGGCTGAAGACTATGATGATGATCTGACTGATTCAGAGATGGATGTTGTGAGATTTGATGATGACAACAGCCCCAGCTTCATCCAGATCAGATCAGTGGCCAAGAAGCACCCCAAGACCTGGGTGCACT |
| | ACATAGCTGCAGAGGAGGAGGACTGGGACTATGCCCCCCTGGTGCTGGCCCCAGATGATAGAAGCTACAAGAGCCAGTACCTGAACAATGGCCCCCAGAGAATAGGGAGAAAGTACAAGAAGGTGAGATTCATGGCCTACACAGA |
| | TGAGACCTTCAAGACCAGAGAGGCCATCCAGCATGAGTCAGGTATCCTGGGCCCCCTGCTGTATGGGGAGGTGGGTGACACCCTGCTGATCATCTTCAAGAACCAGGCCAGCAGACCCTACAACATCTACCCCCATGGGATCACA |
| | GATGTCAGACCCCTGTACAGCAGAAGACTGCCCAAGGGAGTCAAGCACCTGAAGGACTTCCCCATCCTGCCTGGAGAAATCTTCAAGTACAAGTGGACAGTGACTGTGGAGGATGGTCCCACCAAGAGTGACCCCAGATGCCTGA |
| | CCAGATACTACAGCAGCTTTGTCAACATGGAGAGAGACCTGGCCAGTGGCCTGATAGGCCCCCTGCTGATCTGCTACAAGGAGTCTGTGGACCAGCAGGGCAACCAGATCATGTCTGACAAGAGAAATGTTATCCTGTTCAGTGT |
| | ATTTGATGAGAACAGAAGCTGGTACCTGACTGAGAACATCCAGAGATTCCTGCCCAACCCAGCTGGGGTCCAGCTGGAGGACCCTGAGTTCCAGGCCAGCAACATCATGCACAGCATCAATGGCTATGTGTTTGACAGCCTGCAG |
| | CTGTCTGTCTGCCTGCATGAAGTGGCCTACTGGTACATCCTGAGCATAGGAGCCCAGACAGACTTCCTGAGTGTATTCTTCTCTGGCTACACCTTCAAGCACAAGATGGTGTATGAGGACACCCTGACCCTGTTCCCCTTCTCTG |
| | GAGAGACTGTATTCATGAGCATGGAGAACCCTGGCCTGTGGATCCTGGGCTGCCACAACTCTGACTTCAGAAACAGAGGCATGACTGCCCTGCTGAAGGTGAGCAGCTGTGATAAGAACACAGGGGACTACTATGAGGACAGCTA |
| | TGAGGACATCTCAGCCTACCTGCTGAGCAAGAACAATGCCATTGAGCCCAGAAGCTTCAGCCAGAACCCCCCTGTGCTGAAGAGACACCAGAGAGAGATCACCAGAACCACCCTGCAGAGTGACCAGGAGGAGATTGACTATGAT |
| | GACACCATCTCTGTTGAGATGAAGAAGGAGGACTTTGACATCTATGATGAGGATGAGAACCAGAGCCCCAGAAGCTTCCAGAAGAAGACCAGACACTACTTCATTGCAGCTGTGGAGAGACTGTGGGACTATGGCATGAGCAGCA |
| | GCCCCCATGTGCTGAGAAACAGAGCCCAGTCAGGCTCTGTGCCCCAGTTCAAGAAGGTGGTGTTCCAGGAGTTCACAGATGGGAGCTTCACCCAGCCCCTGTACAGAGGTGAACTGAATGAGCACCTGGGCCTGCTGGGCCCCTA |
| | CATCAGAGCTGAAGTGGAGGACAACATCATGGTGACCTTCAGAAACCAGGCCAGCAGACCCTACAGCTTCTACAGCAGCCTGATCAGCTATGAAGAGGACCAGAGACAGGGTGCTGAGCCCAGAAAGAACTTTGTGAAGCCCAAT |
| | GAAACCAAGACCTACTTCTGGAAGGTGCAGCACCACATGGCCCCCACCAAGGATGAATTTGACTGCAAGGCCTGGGCCTACTTCTCAGATGTAGACCTGGAGAAGGATGTTCACTCAGGCCTGATTGGGCCCCTGCTGGTGTGCC |
| | ACACCAACACCCTGAACCCAGCCCATGGTAGACAGGTGACAGTGCAGGAGTTTGCCCTGTTCTTCACCATCTTTGATGAGACCAAGAGCTGGTACTTCACTGAGAACATGGAGAGAAACTGCAGAGCCCCCTGCAACATCCAGAT |
| | GGAGGACCCCACCTTCAAGGAGAACTACAGATTCCATGCCATCAATGGGTACATCATGGACACCCTGCCAGGCCTGGTGATGGCCCAGGACCAGAGAATCAGATGGTACCTGCTGAGCATGGGCAGCAATGAGAACATCCACAGC |
| | ATCCACTTCAGTGGTCATGTCTTCACAGTGAGAAAGAAGGAGGAGTACAAGATGGCCCTGTACAACCTGTACCCAGGGGTGTTTGAAACTGTAGAGATGCTGCCCAGCAAGGCAGGCATCTGGAGAGTGGAGTGCCTGATAGGAG |
| | AACACCTGCATGCAGGAATGAGCACCCTGTTCCTGGTGTACAGCAACAAGTGCCAGACCCCCCTGGGCATGGCCTCTGGGCACATCAGAGACTTCCAGATCACTGCCTCTGGCCAGTATGGGCAGTGGGCCCCCAAGCTGGCCAG |
| | ACTGCACTACTCAGGTAGCATCAATGCCTGGAGCACCAAGGAGCCCTTCAGCTGGATCAAGGTGGACCTGCTGGCCCCCATGATCATCCATGGTATCAAGACCCAGGGTGCCAGACAGAAGTTCAGCAGCCTGTACATCAGCCAG |
| | TTCATCATCATGTACAGCCTGGATGGCAAGAAGTGGCAGACCTACAGAGGCAACAGCACTGGCACCCTGATGGTGTTCTTTGGCAATGTGGACAGCTCTGGCATCAAGCACAACATCTTCAACCCCCCCATCATAGCCAGATACA |
| | TCAGACTGCACCCCACCCACTACAGCATCAGAAGCACCCTGAGAATGGAGCTGATGGGCTGTGATCTGAACAGCTGCAGCATGCCCCTGGGCATGGAGAGCAAGGCCATCTCTGATGCTCAGATCACTGCAAGCAGCTACTTCAC |
| | CAACATGTTTGCCACCTGGAGCCCCAGCAAGGCCAGACTGCACCTGCAGGGCAGAAGCAATGCCTGGAGACCCCAGGTGAACAACCCCAAGGAGTGGCTGCAGGTGGACTTCCAGAAGACCATGAAGGTGACAGGTGTCACCACC |
| | CAGGGGGTGAAGAGCCTGCTGACCAGCATGTATGTGAAGGAGTTCCTGATCAGCAGCAGCCAGGATGGCCACCAGTGGACCCTGTTCTTCCAGAATGGCAAGGTGAAGGTGTTCCAGGGCAACCAGGACAGCTTCACCCCTGTGG |
| | TGAACAGCCTGGACCCCCCCCTGCTGACCAGATACCTGAGAATCCACCCCCAGAGCTGGGTGCACCAGATTGCCCTGAGAATGGAGGTGCTGGGCTGTGAGGCCCAGGACCTGTACTGATTTAAATCTCGAGCCGGGCGGAGTGT |
| | GTTAGTCTCTCCAGAGGGAGGCTGGTTCCCCAGGGAAGCAGAGCCTGTGTGCGGGCAGCAGCTGTGTGCGGGCCTGGGGGTTGTTAAGTGCAATTATTTTTAATAAAAGGGGCATTTGGAAAAAAAAAAAAAAGGTAGCAGTCGA |
| | CAGATGAATTCTGCAGATCTGTGGCTTCTAGCTGCCCGGGTGGCATCCCTGTGACCCCTCCCCAGTGCCTCTCCTGGCCCTGGAAGTTGCCACTCCAGTGCCCACCAGCCTTGTCCTAATAAAATTAAGTTGCATCATTTTGTCT |
| | GACTAGGTGTCCTTCTATAATATTATGGGGTGGAGGGGGGTGGTATGGAGCAAGGGGCAAGTTGGGAAGACAACCTGTAGGGCCTGCGGGGTCTATTGGGAACCAAGCTGGAGTGCAGTGGCACAATCTTGGCTCACTGCAATCT |
| | CCGCCTCCTGGGTTCAAGCGATTCTCCTGCCTCAGCCTCCCGAGTTGTTGGGATTCCAGGCATGCATGACCAGGCTCAGCTAATTTTTGTTTTTTTGGTAGAGACGGGGTTTCACCATATTGGCCAGGCTGGTCTCCAACTCCTA |
| | ATCTCAGGTGATCTACCCACCTTGGCCTCCCAAATTGCTGGGATTACAGGCGTGAACCACTGCTCCCTTCCCTGTCCTTCTGATTTTAAAAAAAAGAGAAATGTTCTGGCACCTGCACTTGCACTGGGGACAGCCTATTTTGCTA |
| | GTTTGTTTTGTTTCGTTTTGTTTTGATGGAGAGCGTATGTTGTTTAAACGCGGCCGCGTAGATAAGTAGCATGGCGGGTTAATCATTAACTACAAGGAACCCCTAGTGATGGAGTTGGCCACTCCCTCTCTGCGCGCTCGCTCGC |
| | TCACTGAGGCCGGGCGACCAAAGGTCGCCCGACGCCCGGGCTTTGCCCGGGCGGCCTCAGTGAGCGAGCGAGCGCGCAG |
| 27 | >ITR-to-ITR construct with FVIII-QQ2 nucleic acid |
| | >[L-ITR]-[ACTUS101-LSP]-[5′ UTR]-[FVIII-QQ2(w/sp)]-[3′ UTR]-[PolIII_terminator]-[hGHpolyA]-[PolII_terminator-rev] [R-ITR] |
| | CTGCGCGCTCGCTCGCTCACTGAGGCCGCCCGGGCAAAGCCCGGGCGTCGGGCGACCTTTGGTCGCCCGGCCTCAGTGAGCGAGCGAGCGCGCAGAGAGGGAGTGGCCAACTCCATCACTAGGGGTTCCTTGTAGTTAATGATTA |
| | ACCCGCCATGCTACTTATCTACGGCGCGCCACGCGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACT |
| | AGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACTAGTTCTAGAGCGGCCGCCAGTGTGCTGGAATTC |
| | GGCTTTTTTAGGGCTGGAAGCTACCTTTGACATCATTTCCTCTGCGAATGCATGTATAATTTCTACAGAACCTATTAGAAAGGATCACCCAGCCTCTGCTTTTGTACAACTTTCCCTTAAAAAACTGCCAATCCCACTGCTGTTT |
| | GGCCCAATAGTGAGAACTTTTTCCTGCTGCCTCTTGGTGCTTTTGCCTATGGCCCCTATTCTGCCTGCTGAAGACACTCTTGCCAGCATGGACTTAAACCCCTCCAGCTCTGACAATCCTCTTTCTCTTTTGTTTTACATGAAGG |
| | GTCTGGCAGCCAAAGCAATCACTCAAAGTTCAAACCTTATCATTTTTTGCTTTGTTCCTCTTGGCCTTGGTTTTGTACATCAGCTTTGAAAATACCATCCCAGGGTTAATGCTGGGGTTAATTTATAACTGAGAGTGCTCTAGTT |
| | CTGCAATACAGGACATGCTATAAAAATGGAAAGATGTTGCTTTCTGAGAGATCAGGTGGATTCTTGGGCATTTGCTTAAGCTTGGTACCGCCACCATGCAGATTGAGCTGAGCACCTGCTTCTTCCTGTGCCTGCTGAGATTCTG |
| | CTTCAGTGCCACCAGAAGATACTACCTGGGAGCAGTGGAGCTGAGCTGGGACTACATGCAGAGTGATCTGGGAGAGCTGCCAGTAGATGCCAGATTCCCCCCCAGAGTGCCCAAGAGCTTCCCCTTCAACACCTCAGTGGTGTAC |
| | AAGAAGACCCTGTTTGTTGAGTTCACTGACCACCTGTTCAACATTGCAAAGCCCAGACCCCCCTGGATGGGCCTGCTGGGCCCCACCATCCAGGCAGAAGTGTATGACACAGTAGTGATCACCCTGAAGAACATGGCCAGCCACC |
| | CTGTCAGCCTGCATGCAGTTGGTGTAAGCTACTGGAAGGCCTCTGAGGGTGCTGAATATGATGACCAGACCAGCCAGAGAGAGAAGGAGGATGATAAGGTGTTCCCAGGGGGCAGCCACACCTATGTTTGGCAGGTGCTGAAGGA |
| | GAATGGGCCCATGGCCTCTGATCCCCTGTGCCTGACCTACAGCTACCTGAGCCATGTGGACCTGGTGAAGGACCTGAACTCTGGGCTGATAGGAGCCCTGCTGGTGTGCAGAGAGGGCAGCCTGGCCAAGGAGAAGACCCAGACC |
| | CTGCACAAGTTCATCCTGCTGTTTGCTGTGTTTGATGAAGGCAAGAGCTGGCACAGTGAAACCAAGAACAGCCTGATGCAGGACAGAGATGCAGCTAGTGCCAGAGCCTGGCCCAAGATGCACACAGTAAATGGTTATGTGAACA |
| | GAAGCCTGCCTGGCCTGATAGGCTGCCACAGAAAGTCTGTGTACTGGCATGTTATAGGTATGGGCACCACCCCTGAGGTGCACAGCATCTTCCTGGAGGGCCACACCTTCCTGGTGAGAAACCACAGACAGGCCAGCCTGGAGAT |
| | CAGCCCCATCACCTTCCTGACTGCCCAGACCCTGCTGATGGACCTGGGCCAGTTCCTGCTGTTCTGCCACATCAGCAGCCACCAGCATGATGGAATGGAGGCCTATGTCAAGGTGGACAGCTGCCCAGAGGAGCCCCAGCTGCAG |
| | ATGAAGAACAATGAGGAGGCAGAGGACTATGATGATGACCTGACTGACAGTGAAATGGATGTGGTCAGATTTGATGATGATAACAGCCCCAGCTTCATCCAGATCAGATCAGTGGCCAAGAAGCACCCCAAGACCTGGGTGCACT |
| | ACATTGCAGCTGAGGAGGAGGACTGGGACTATGCACCCCTGGTGCTGGCCCCAGATGATAGAAGCTACAAGAGCCAGTACCTGAACAATGGACCCCAGAGAATTGGCAGAAAGTACAAGAAGGTGAGATTCATGGCCTACACTGA |
| | TGAGACCTTCAAGACCAGAGAGGCCATCCAGCATGAATCAGGCATCCTGGGCCCCCTGCTGTATGGGGAGGTGGGAGACACCCTGCTGATCATCTTCAAGAACCAGGCCAGCAGACCCTACAACATCTACCCCCATGGAATCACA |
| | GATGTTAGACCCCTGTACAGCAGAAGACTGCCCAAGGGGGTGAAGCACCTGAAGGACTTCCCCATCCTGCCTGGGGAGATCTTCAAGTACAAGTGGACAGTTACAGTAGAGGATGGCCCCACCAAGAGTGACCCCAGATGCCTGA |
| | CCAGATACTACAGCAGCTTTGTAAACATGGAGAGAGACCTGGCCTCTGGCCTGATAGGCCCCCTGCTGATCTGCTACAAGGAGTCAGTTGACCAGCAGGGCAACCAGATCATGAGTGATAAGAGAAATGTCATCCTGTTCAGTGT |
| | GTTTGATGAGAACAGAAGCTGGTACCTGACAGAGAACATCCAGAGATTCCTGCCCAACCCTGCTGGAGTGCAGCTGGAGGACCCAGAATTCCAGGCCAGCAACATCATGCACAGCATCAATGGTTATGTCTTTGATAGCCTGCAG |
| | CTGTCAGTTTGCCTGCATGAGGTGGCCTACTGGTACATCCTGAGCATTGGGGCCCAGACTGACTTCCTGAGTGTATTCTTCAGTGGCTACACCTTCAAGCACAAGATGGTGTATGAGGACACCCTGACCCTGTTCCCCTTCTCTG |
| | GAGAGACTGTGTTCATGAGCATGGAGAACCCTGGGCTGTGGATCCTGGGCTGCCACAACTCAGACTTCAGAAACAGAGGCATGACAGCACTGCTGAAGGTGAGCAGCTGTGATAAGAACACTGGTGACTACTATGAGGACAGCTA |
| | TGAAGACATCTCAGCCTACCTGCTGAGCAAGAACAATGCCATAGAGCCCAGAAGCTTCAGCCAGAACCCCCCAGTTCTGAAGAGACACCAGAGAGAGATCACCAGAACCACCCTGCAGAGTGACCAGGAGGAGATAGATTATGAT |
| | GATACCATCTCAGTTGAGATGAAGAAGGAGGACTTTGATATCTATGATGAGGATGAGAACCAGAGCCCCAGAAGCTTCCAGAAGAAGACCAGACACTACTTCATAGCAGCTGTGGAGAGACTGTGGGACTATGGCATGAGCAGCA |
| | GCCCCCATGTTCTGAGAAACAGAGCCCAGAGTGGATCAGTACCCCAGTTCAAGAAGGTGGTGTTCCAGGAGTTCACTGATGGGAGCTTCACCCAGCCCCTGTACAGAGGTGAACTGAATGAGCACCTGGGCCTGCTGGGCCCCTA |
| | CATCAGAGCTGAAGTGGAGGACAACATCATGGTGACCTTCAGAAACCAGGCCAGCAGACCCTACAGCTTCTACAGCAGCCTGATCAGCTATGAGGAGGACCAGAGACAGGGAGCTGAGCCCAGAAAGAACTTTGTCAAGCCCAAT |
| | GAAACCAAGACCTACTTCTGGAAGGTGCAGCACCACATGGCCCCCACCAAGGATGAGTTTGACTGCAAGGCCTGGGCCTACTTCAGTGATGTAGACCTGGAGAAGGATGTCCACTCAGGCCTGATTGGCCCCCTGCTGGTGTGCC |
| | ACACCAACACCCTGAACCCAGCACATGGGAGACAGGTGACTGTACAGGAGTTTGCTCTGTTCTTCACCATCTTTGATGAGACCAAGAGCTGGTACTTCACAGAAAACATGGAGAGAAACTGCAGAGCCCCCTGCAACATCCAGAT |
| | GGAGGACCCCACCTTCAAGGAGAACTACAGATTCCATGCCATCAATGGTTACATCATGGACACCCTGCCTGGCCTGGTGATGGCCCAGGACCAGAGAATCAGATGGTACCTGCTGAGCATGGGCAGCAATGAAAACATCCACAGC |
| | ATCCACTTCTCAGGGCATGTGTTCACTGTGAGAAAGAAGGAGGAGTACAAGATGGCCCTGTACAACCTGTACCCAGGTGTGTTTGAAACTGTGGAGATGCTGCCCAGCAAGGCAGGCATCTGGAGAGTGGAGTGCCTGATAGGGG |
| | AGCACCTGCATGCAGGGATGAGCACCCTGTTCCTGGTGTACAGCAACAAGTGCCAGACCCCCCTGGGCATGGCCTCAGGCCACATCAGAGACTTCCAGATCACAGCTTCTGGACAGTATGGCCAGTGGGCCCCCAAGCTGGCCAG |
| | ACTGCACTACAGTGGCAGCATCAATGCCTGGAGCACCAAGGAGCCCTTCAGCTGGATCAAGGTGGACCTGCTGGCCCCCATGATCATCCATGGCATCAAGACCCAGGGAGCCAGACAGAAGTTCAGCAGCCTGTACATCAGCCAG |
| | TTCATCATCATGTACAGCCTGGATGGCAAGAAGTGGCAGACCTACAGAGGCAACAGCACAGGCACCCTGATGGTGTTCTTTGGGAATGTGGACAGCAGTGGAATCAAGCACAACATCTTCAACCCCCCCATCATTGCAAGATACA |
| | TCAGACTGCACCCCACCCACTACAGCATCAGAAGCACCCTGAGAATGGAGCTGATGGGCTGTGACCTGAACAGCTGCAGCATGCCCCTGGGCATGGAGAGCAAGGCCATCTCTGATGCCCAGATCACAGCTAGCAGCTACTTCAC |
| | CAACATGTTTGCCACCTGGAGCCCCAGCAAGGCCAGACTGCACCTGCAGGGCAGAAGCAATGCCTGGAGACCCCAGGTGAACAACCCCAAGGAGTGGCTGCAGGTGGACTTCCAGAAGACCATGAAGGTGACTGGTGTTACCACC |
| | CAGGGGGTCAAGAGCCTGCTGACCAGCATGTATGTGAAGGAGTTCCTGATCAGCAGCAGCCAGGATGGCCACCAGTGGACCCTGTTCTTCCAGAATGGCAAGGTGAAGGTGTTCCAGGGCAACCAGGACAGCTTCACCCCTGTGG |
| | TGAACAGCCTGGACCCCCCCCTGCTGACCAGATACCTGAGAATCCACCCCCAGAGCTGGGTGCACCAGATAGCACTGAGAATGGAGGTGCTGGGCTGTGAGGCCCAGGACCTGTACTGATTTAAATCTCGAGCCGGGCGGAGTGT |
| | GTTAGTCTCTCCAGAGGGAGGCTGGTTCCCCAGGGAAGCAGAGCCTGTGTGCGGGCAGCAGCTGTGTGCGGGCCTGGGGGTTGTTAAGTGCAATTATTTTTAATAAAAGGGGCATTTGGAAAAAAAAAAAAAAGGTAGCAGTCGA |
| | CAGATGAATTCTGCAGATCTGTGGCTTCTAGCTGCCCGGGTGGCATCCCTGTGACCCCTCCCCAGTGCCTCTCCTGGCCCTGGAAGTTGCCACTCCAGTGCCCACCAGCCTTGTCCTAATAAAATTAAGTTGCATCATTTTGTCT |
| | GACTAGGTGTCCTTCTATAATATTATGGGGTGGAGGGGGGTGGTATGGAGCAAGGGGCAAGTTGGGAAGACAACCTGTAGGGCCTGCGGGGTCTATTGGGAACCAAGCTGGAGTGCAGTGGCACAATCTTGGCTCACTGCAATCT |
| | CCGCCTCCTGGGTTCAAGCGATTCTCCTGCCTCAGCCTCCCGAGTTGTTGGGATTCCAGGCATGCATGACCAGGCTCAGCTAATTTTTGTTTTTTTGGTAGAGACGGGGTTTCACCATATTGGCCAGGCTGGTCTCCAACTCCTA |
| | ATCTCAGGTGATCTACCCACCTTGGCCTCCCAAATTGCTGGGATTACAGGCGTGAACCACTGCTCCCTTCCCTGTCCTTCTGATTTTAAAAAAAAGAGAAATGTTCTGGCACCTGCACTTGCACTGGGGACAGCCTATTTTGCTA |
| | GTTTGTTTTGTTTCGTTTTGTTTTGATGGAGAGCGTATGTTGTTTAAACGCGGCCGCGTAGATAAGTAGCATGGCGGGTTAATCATTAACTACAAGGAACCCCTAGTGATGGAGTTGGCCACTCCCTCTCTGCGCGCTCGCTCGC |
| | TCACTGAGGCCGGGCGACCAAAGGTCGCCCGACGCCCGGGCTTTGCCCGGGCGGCCTCAGTGAGCGAGCGAGCGCGCAG |
| 28 | >ITR-to-ITR construct with FVIII-QQ3 nucleic acid |
| | >[L-ITR]-[ACTUS101-LSP]-[5′ UTR]-[FVIII-QQ3(w/sp)]-[3′ UTR]-[PolIII_terminator]-[hGHpolyA]-[PolII_terminator-rev][R-ITR] |
| | CTGCGCGCTCGCTCGCTCACTGAGGCCGCCCGGGCAAAGCCCGGGCGTCGGGCGACCTTTGGTCGCCCGGCCTCAGTGAGCGAGCGAGCGCGCAGAGAGGGAGTGGCCAACTCCATCACTAGGGGTTCCTTGTAGTTAATGATTA |
| | ACCCGCCATGCTACTTATCTACGGCGCGCCACGCGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACT |
| | AGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACTAGTTCTAGAGCGGCCGCCAGTGTGCTGGAATTC |
| | GGCTTTTTTAGGGCTGGAAGCTACCTTTGACATCATTTCCTCTGCGAATGCATGTATAATTTCTACAGAACCTATTAGAAAGGATCACCCAGCCTCTGCTTTTGTACAACTTTCCCTTAAAAAACTGCCAATCCCACTGCTGTTT |
| | GGCCCAATAGTGAGAACTTTTTCCTGCTGCCTCTTGGTGCTTTTGCCTATGGCCCCTATTCTGCCTGCTGAAGACACTCTTGCCAGCATGGACTTAAACCCCTCCAGCTCTGACAATCCTCTTTCTCTTTTGTTTTACATGAAGG |
| | GTCTGGCAGCCAAAGCAATCACTCAAAGTTCAAACCTTATCATTTTTTGCTTTGTTCCTCTTGGCCTTGGTTTTGTACATCAGCTTTGAAAATACCATCCCAGGGTTAATGCTGGGGTTAATTTATAACTGAGAGTGCTCTAGTT |
| | CTGCAATACAGGACATGCTATAAAAATGGAAAGATGTTGCTTTCTGAGAGATCAGGTGGATTCTTGGGCATTTGCTTAAGCTTGGTACCGCCACCATGCAAATTGAGTTGTCCACCTGCTTCTTCTTGTGCTTGTTGAGGTTCTG |
| | CTTCAGTGCCACCAGGAGGTACTACTTGGGGGCTGTGGAGTTGTCCTGGGACTACATGCAGTCAGACTTGGGGGAGTTGCCTGTGGATGCCAGGTTCCCCCCCAGGGTCCCCAAGTCCTTCCCCTTCAACACCAGTGTGGTCTAC |
| | AAGAAGACCTTGTTTGTGGAGTTCACTGACCACTTGTTCAACATTGCCAAGCCCAGGCCCCCCTGGATGGGCTTGTTGGGCCCCACCATCCAGGCTGAGGTCTATGACACAGTGGTCATCACCTTGAAGAACATGGCCTCCCACC |
| | CTGTCTCCTTGCATGCAGTGGGGGTCTCCTACTGGAAGGCCAGTGAGGGGGCTGAGTATGATGACCAGACCTCCCAGAGGGAGAAGGAGGATGACAAGGTCTTCCCTGGGGGCTCCCACACCTATGTCTGGCAGGTCTTGAAGGA |
| | GAATGGCCCCATGGCCTCTGACCCCTTGTGCTTGACCTACTCCTACTTGTCCCATGTGGACTTGGTCAAGGACTTGAACTCAGGCTTGATTGGGGCCTTGTTGGTCTGCAGGGAGGGCTCCTTGGCCAAGGAGAAGACCCAGACC |
| | TTGCACAAGTTCATCTTGTTGTTTGCAGTCTTTGATGAGGGCAAGTCCTGGCACAGTGAGACCAAGAACTCCTTGATGCAGGACAGGGATGCTGCCAGTGCCAGGGCCTGGCCCAAGATGCACACTGTCAATGGCTATGTCAACA |
| | GGTCCTTGCCTGGCTTGATTGGCTGCCACAGGAAGTCAGTCTACTGGCATGTCATTGGCATGGGCACCACCCCTGAGGTCCACTCCATCTTCTTGGAGGGCCACACCTTCTTGGTCAGGAACCACAGGCAGGCCTCCTTGGAGAT |
| | CTCCCCCATCACCTTCTTGACAGCCCAGACCTTGTTGATGGACTTGGGCCAGTTCTTGTTGTTCTGCCACATCTCCTCCCACCAGCATGATGGCATGGAGGCCTATGTCAAGGTGGACTCCTGCCCTGAGGAGCCCCAGTTGCAG |
| | ATGAAGAACAATGAGGAGGCAGAGGACTATGATGATGACTTGACTGACTCTGAGATGGATGTGGTCAGGTTTGATGATGACAACTCCCCCTCCTTCATCCAGATCAGGAGTGTGGCCAAGAAGCACCCCAAGACCTGGGTCCACT |
| | ACATAGCAGCAGAGGAGGAGGACTGGGACTATGCCCCCTTGGTCTTGGCCCCTGATGACAGGTCCTACAAGTCCCAGTACTTGAACAATGGCCCCCAGAGGATTGGCAGGAAGTACAAGAAGGTCAGGTTCATGGCCTACACAGA |
| | TGAGACCTTCAAGACCAGGGAGGCCATCCAGCATGAGTCAGGCATCTTGGGCCCCTTGTTGTATGGGGAGGTGGGGGACACCTTGTTGATCATCTTCAAGAACCAGGCCTCCAGGCCCTACAACATCTACCCCCATGGCATCACT |
| | GATGTCAGGCCCTTGTACTCCAGGAGGTTGCCCAAGGGGGTCAAGCACTTGAAGGACTTCCCCATCTTGCCAGGGGAGATCTTCAAGTACAAGTGGACAGTCACTGTGGAGGATGGCCCCACCAAGTCAGACCCCAGGTGCTTGA |
| | CCAGGTACTACTCCTCCTTTGTCAACATGGAGAGGGACTTGGCCAGTGGCTTGATTGGCCCCTTGTTGATCTGCTACAAGGAGTCTGTGGACCAGCAGGGCAACCAGATCATGTCAGACAAGAGGAATGTCATCTTGTTCAGTGT |
| | CTTTGATGAGAACAGGTCCTGGTACTTGACAGAGAACATCCAGAGGTTCTTGCCCAACCCAGCTGGGGTCCAGTTGGAGGACCCTGAGTTCCAGGCCTCCAACATCATGCACTCCATCAATGGCTATGTCTTTGACTCCTTGCAG |
| | TTGTCAGTCTGCTTGCATGAGGTGGCCTACTGGTACATCTTGTCCATTGGGGCCCAGACTGACTTCTTGTCTGTCTTCTTCAGTGGCTACACCTTCAAGCACAAGATGGTCTATGAGGACACCTTGACCTTGTTCCCCTTCAGTG |
| | GGGAGACTGTCTTCATGTCCATGGAGAACCCAGGCTTGTGGATCTTGGGCTGCCACAACTCAGACTTCAGGAACAGGGGCATGACAGCCTTGTTGAAGGTCTCCTCCTGTGACAAGAACACAGGGGACTACTATGAGGACTCCTA |
| | TGAGGACATCTCAGCCTACTTGTTGTCCAAGAACAATGCCATAGAGCCCAGGTCCTTCTCCCAGAACCCCCCTGTCTTGAAGAGGCACCAGAGGGAGATCACCAGGACCACCTTGCAGTCAGACCAGGAGGAGATAGACTATGAT |
| | GACACCATCTCAGTGGAGATGAAGAAGGAGGACTTTGACATCTATGATGAGGATGAGAACCAGTCCCCCAGGTCCTTCCAGAAGAAGACCAGGCACTACTTCATTGCAGCAGTGGAGAGGTTGTGGGACTATGGCATGTCCTCCT |
| | CCCCCCATGTCTTGAGGAACAGGGCCCAGTCAGGCAGTGTCCCCCAGTTCAAGAAGGTGGTCTTCCAGGAGTTCACTGATGGCTCCTTCACCCAGCCCTTGTACAGGGGGGAGTTGAATGAGCACTTGGGCTTGTTGGGCCCCTA |
| | CATCAGGGCTGAGGTGGAGGACAACATCATGGTCACCTTCAGGAACCAGGCCTCCAGGCCCTACTCCTTCTACTCCTCCTTGATCTCCTATGAGGAGGACCAGAGGCAGGGGGCAGAGCCCAGGAAGAACTTTGTCAAGCCCAAT |
| | GAGACCAAGACCTACTTCTGGAAGGTCCAGCACCACATGGCCCCCACCAAGGATGAGTTTGACTGCAAGGCCTGGGCCTACTTCTCTGATGTGGACTTGGAGAAGGATGTCCACTCTGGCTTGATTGGCCCCTTGTTGGTCTGCC |
| | ACACCAACACCTTGAACCCTGCCCATGGCAGGCAGGTCACTGTCCAGGAGTTTGCCTTGTTCTTCACCATCTTTGATGAGACCAAGTCCTGGTACTTCACAGAGAACATGGAGAGGAACTGCAGGGCCCCCTGCAACATCCAGAT |
| | GGAGGACCCCACCTTCAAGGAGAACTACAGGTTCCATGCCATCAATGGCTACATCATGGACACCTTGCCAGGCTTGGTCATGGCCCAGGACCAGAGGATCAGGTGGTACTTGTTGTCCATGGGCTCCAATGAGAACATCCACTCC |
| | ATCCACTTCAGTGGCCATGTCTTCACTGTCAGGAAGAAGGAGGAGTACAAGATGGCCTTGTACAACTTGTACCCAGGGGTCTTTGAGACAGTGGAGATGTTGCCCTCCAAGGCAGGCATCTGGAGGGTGGAGTGCTTGATTGGGG |
| | AGCACTTGCATGCTGGCATGTCCACCTTGTTCTTGGTCTACTCCAACAAGTGCCAGACCCCCTTGGGCATGGCCTCTGGCCACATCAGGGACTTCCAGATCACTGCCTCAGGCCAGTATGGCCAGTGGGCCCCCAAGTTGGCCAG |
| | GTTGCACTACAGTGGCTCCATCAATGCCTGGTCCACCAAGGAGCCCTTCTCCTGGATCAAGGTGGACTTGTTGGCCCCCATGATCATCCATGGCATCAAGACCCAGGGGGCCAGGCAGAAGTTCTCCTCCTTGTACATCTCCCAG |
| | TTCATCATCATGTACTCCTTGGATGGCAAGAAGTGGCAGACCTACAGGGGCAACTCCACAGGCACCTTGATGGTCTTCTTTGGCAATGTGGACTCCTCTGGCATCAAGCACAACATCTTCAACCCCCCCATCATTGCCAGGTACA |
| | TCAGGTTGCACCCCACCCACTACTCCATCAGGTCCACCTTGAGGATGGAGTTGATGGGCTGTGACTTGAACTCCTGCTCCATGCCCTTGGGCATGGAGTCCAAGGCCATCTCAGATGCCCAGATCACTGCCTCCTCCTACTTCAC |
| | CAACATGTTTGCCACCTGGTCCCCCTCCAAGGCCAGGTTGCACTTGCAGGGCAGGTCCAATGCCTGGAGGCCCCAGGTCAACAACCCCAAGGAGTGGTTGCAGGTGGACTTCCAGAAGACCATGAAGGTCACAGGGGTCACCACC |
| | CAGGGGGTCAAGTCCTTGTTGACCTCCATGTATGTCAAGGAGTTCTTGATCTCCTCCTCCCAGGATGGCCACCAGTGGACCTTGTTCTTCCAGAATGGCAAGGTCAAGGTCTTCCAGGGCAACCAGGACTCCTTCACCCCTGTGG |
| | TCAACTCCTTGGACCCCCCCTTGTTGACCAGGTACTTGAGGATCCACCCCCAGTCCTGGGTCCACCAGATTGCCTTGAGGATGGAGGTCTTGGGCTGTGAGGCCCAGGACTTGTACTGATTTAAATCTCGAGCCGGGCGGAGTGT |
| | GTTAGTCTCTCCAGAGGGAGGCTGGTTCCCCAGGGAAGCAGAGCCTGTGTGCGGGCAGCAGCTGTGTGCGGGCCTGGGGGTTGTTAAGTGCAATTATTTTTAATAAAAGGGGCATTTGGAAAAAAAAAAAAAAGGTAGCAGTCGA |
| | CAGATGAATTCTGCAGATCTGTGGCTTCTAGCTGCCCGGGTGGCATCCCTGTGACCCCTCCCCAGTGCCTCTCCTGGCCCTGGAAGTTGCCACTCCAGTGCCCACCAGCCTTGTCCTAATAAAATTAAGTTGCATCATTTTGTCT |
| | GACTAGGTGTCCTTCTATAATATTATGGGGTGGAGGGGGGTGGTATGGAGCAAGGGGCAAGTTGGGAAGACAACCTGTAGGGCCTGCGGGGTCTATTGGGAACCAAGCTGGAGTGCAGTGGCACAATCTTGGCTCACTGCAATCT |
| | CCGCCTCCTGGGTTCAAGCGATTCTCCTGCCTCAGCCTCCCGAGTTGTTGGGATTCCAGGCATGCATGACCAGGCTCAGCTAATTTTTGTTTTTTTGGTAGAGACGGGGTTTCACCATATTGGCCAGGCTGGTCTCCAACTCCTA |
| | ATCTCAGGTGATCTACCCACCTTGGCCTCCCAAATTGCTGGGATTACAGGCGTGAACCACTGCTCCCTTCCCTGTCCTTCTGATTTTAAAAAAAAGAGAAATGTTCTGGCACCTGCACTTGCACTGGGGACAGCCTATTTTGCTA |
| | GTTTGTTTTGTTTCGTTTTGTTTTGATGGAGAGCGTATGTTGTTTAAACGCGGCCGCGTAGATAAGTAGCATGGCGGGTTAATCATTAACTACAAGGAACCCCTAGTGATGGAGTTGGCCACTCCCTCTCTGCGCGCTCGCTCGC |
| | TCACTGAGGCCGGGCGACCAAAGGTCGCCCGACGCCCGGGCTTTGCCCGGGCGGCCTCAGTGAGCGAGCGAGCGCGCAG |
| 29 | >ITR-to-ITR construct with FVIII-QQ4 nucleic acid |
| | >[L-ITR]-[ACTUS101-LSP]-[5′ UTR]-[FVIII-QQ4(w/sp)]-[3′ UTR]-[PolIII_terminator]-[hGHpolyA]-[PolII_terminator-rev][R-ITR] |
| | CTGCGCGCTCGCTCGCTCACTGAGGCCGCCCGGGCAAAGCCCGGGCGTCGGGCGACCTTTGGTCGCCCGGCCTCAGTGAGCGAGCGAGCGCGCAGAGAGGGAGTGGCCAACTCCATCACTAGGGGTTCCTTGTAGTTAATGATTA |
| | ACCCGCCATGCTACTTATCTACGGCGCGCCACGCGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACT |
| | AGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACTAGTTCTAGAGCGGCCGCCAGTGTGCTGGAATTC |
| | GGCTTTTTTAGGGCTGGAAGCTACCTTTGACATCATTTCCTCTGCGAATGCATGTATAATTTCTACAGAACCTATTAGAAAGGATCACCCAGCCTCTGCTTTTGTACAACTTTCCCTTAAAAAACTGCCAATCCCACTGCTGTTT |
| | GGCCCAATAGTGAGAACTTTTTCCTGCTGCCTCTTGGTGCTTTTGCCTATGGCCCCTATTCTGCCTGCTGAAGACACTCTTGCCAGCATGGACTTAAACCCCTCCAGCTCTGACAATCCTCTTTCTCTTTTGTTTTACATGAAGG |
| | GTCTGGCAGCCAAAGCAATCACTCAAAGTTCAAACCTTATCATTTTTTGCTTTGTTCCTCTTGGCCTTGGTTTTGTACATCAGCTTTGAAAATACCATCCCAGGGTTAATGCTGGGGTTAATTTATAACTGAGAGTGCTCTAGTT |
| | CTGCAATACAGGACATGCTATAAAAATGGAAAGATGTTGCTTTCTGAGAGATCAGGTGGATTCTTGGGCATTTGCTTAAGCTTGGTACCGCCACCATGCAGATAGAGCTGAGCACCTGCTTCTTCCTGTGCCTGCTGAGATTCTG |
| | CTTCTCAGCCACCAGAAGATACTACCTGGGGGCTGTAGAGCTGAGCTGGGACTACATGCAGTCAGACCTGGGTGAGCTGCCAGTGGATGCCAGATTCCCCCCCAGAGTGCCCAAGAGCTTCCCCTTCAACACCTCTGTGGTGTAC |
| | AAGAAGACCCTGTTTGTGGAGTTCACTGATCACCTGTTCAACATAGCTAAGCCCAGACCCCCCTGGATGGGCCTGCTGGGCCCCACCATCCAGGCTGAGGTGTATGATACAGTGGTGATCACCCTGAAGAACATGGCCAGCCACC |
| | CTGTGAGCCTGCATGCAGTGGGTGTGAGCTACTGGAAGGCCTCTGAGGGTGCTGAATATGATGACCAGACCAGCCAGAGAGAGAAGGAGGATGATAAGGTGTTCCCTGGGGGCAGCCACACCTATGTGTGGCAGGTGCTGAAGGA |
| | GAATGGACCCATGGCCAGTGACCCCCTGTGCCTGACCTACAGCTACCTGAGCCATGTAGACCTGGTGAAGGACCTGAACTCTGGACTGATTGGGGCACTGCTGGTGTGCAGAGAGGGCAGCCTGGCCAAGGAGAAGACCCAGACC |
| | CTGCACAAGTTCATCCTGCTGTTTGCTGTGTTTGATGAGGGCAAGAGCTGGCACTCTGAAACCAAGAACAGCCTGATGCAGGACAGAGATGCTGCCTCAGCAAGAGCCTGGCCCAAGATGCACACTGTGAATGGCTATGTTAACA |
| | GAAGCCTGCCTGGGCTGATTGGGTGCCACAGAAAGAGTGTGTACTGGCATGTAATAGGAATGGGCACCACCCCAGAGGTGCACAGCATCTTCCTGGAGGGCCACACCTTCCTGGTGAGAAACCACAGACAGGCCAGCCTGGAGAT |
| | CAGCCCCATCACCTTCCTGACTGCTCAGACCCTGCTGATGGACCTGGGCCAGTTCCTGCTGTTCTGCCACATCAGCAGCCACCAGCATGATGGAATGGAGGCCTATGTAAAGGTGGACAGCTGCCCTGAGGAGCCCCAGCTGCAG |
| | ATGAAGAACAATGAGGAGGCAGAGGACTATGATGATGACCTGACTGACAGTGAGATGGATGTGGTGAGATTTGATGATGATAACAGCCCCAGCTTCATCCAGATCAGATCTGTAGCCAAGAAGCACCCCAAGACCTGGGTGCACT |
| | ACATAGCTGCAGAAGAGGAGGACTGGGACTATGCCCCCCTGGTGCTGGCCCCTGATGATAGAAGCTACAAGAGCCAGTACCTGAACAATGGTCCCCAGAGAATAGGGAGAAAGTACAAGAAGGTGAGATTCATGGCCTACACTGA |
| | TGAGACCTTCAAGACCAGAGAGGCCATCCAGCATGAGTCAGGCATCCTGGGCCCCCTGCTGTATGGAGAAGTGGGTGACACCCTGCTGATCATCTTCAAGAACCAGGCCAGCAGACCCTACAACATCTACCCCCATGGCATCACT |
| | GATGTCAGACCCCTGTACAGCAGAAGACTGCCCAAGGGTGTGAAGCACCTGAAGGACTTCCCCATCCTGCCAGGTGAGATCTTCAAGTACAAGTGGACAGTGACTGTAGAGGATGGGCCCACCAAGTCAGACCCCAGATGCCTGA |
| | CCAGATACTACAGCAGCTTTGTGAACATGGAGAGAGACCTGGCCAGTGGCCTGATTGGCCCCCTGCTGATCTGCTACAAGGAGTCAGTTGACCAGCAGGGCAACCAGATCATGTCTGACAAGAGAAATGTCATCCTGTTCAGTGT |
| | GTTTGATGAGAACAGAAGCTGGTACCTGACTGAGAACATCCAGAGATTCCTGCCCAACCCAGCAGGAGTTCAGCTGGAGGACCCTGAGTTCCAGGCCAGCAACATCATGCACAGCATCAATGGCTATGTTTTTGACAGCCTGCAG |
| | CTGTCAGTGTGCCTGCATGAAGTGGCCTACTGGTACATCCTGAGCATTGGTGCCCAGACAGATTTCCTGAGTGTCTTCTTCTCAGGCTACACCTTCAAGCACAAGATGGTGTATGAGGACACCCTGACCCTGTTCCCCTTCTCTG |
| | GGGAAACAGTGTTCATGAGCATGGAGAACCCTGGTCTGTGGATCCTGGGCTGCCACAACTCAGACTTCAGAAACAGAGGCATGACAGCACTGCTGAAGGTGAGCAGCTGTGACAAGAACACAGGTGACTACTATGAAGACAGCTA |
| | TGAGGACATCTCAGCCTACCTGCTGAGCAAGAACAATGCCATTGAGCCCAGAAGCTTCAGCCAGAACCCCCCTGTGCTGAAGAGACACCAGAGAGAGATCACCAGAACCACCCTGCAGTCAGACCAGGAGGAGATTGACTATGAT |
| | GACACCATCAGTGTAGAAATGAAGAAGGAGGACTTTGACATCTATGATGAGGATGAAAACCAGAGCCCCAGAAGCTTCCAGAAGAAGACCAGACACTACTTCATAGCTGCAGTTGAGAGACTGTGGGACTATGGAATGAGCAGCA |
| | GCCCCCATGTGCTGAGAAACAGAGCCCAGTCTGGCTCAGTTCCCCAGTTCAAGAAGGTGGTGTTCCAGGAGTTCACAGATGGGAGCTTCACCCAGCCCCTGTACAGAGGGGAGCTGAATGAGCACCTGGGCCTGCTGGGCCCCTA |
| | CATCAGAGCAGAGGTGGAGGACAACATCATGGTGACCTTCAGAAACCAGGCCAGCAGACCCTACAGCTTCTACAGCAGCCTGATCAGCTATGAAGAGGACCAGAGACAGGGAGCAGAACCCAGAAAGAACTTTGTTAAGCCCAAT |
| | GAAACCAAGACCTACTTCTGGAAGGTGCAGCACCACATGGCCCCCACCAAGGATGAATTTGACTGCAAGGCCTGGGCCTACTTCTCTGATGTTGACCTGGAGAAGGATGTGCACTCAGGCCTGATAGGCCCCCTGCTGGTGTGCC |
| | ACACCAACACCCTGAACCCAGCTCATGGGAGACAGGTGACTGTTCAGGAGTTTGCCCTGTTCTTCACCATCTTTGATGAGACCAAGAGCTGGTACTTCACAGAGAACATGGAGAGAAACTGCAGAGCCCCCTGCAACATCCAGAT |
| | GGAGGACCCCACCTTCAAGGAGAACTACAGATTCCATGCCATCAATGGTTACATCATGGACACCCTGCCTGGGCTGGTGATGGCCCAGGACCAGAGAATCAGATGGTACCTGCTGAGCATGGGCAGCAATGAAAACATCCACAGC |
| | ATCCACTTCTCTGGCCATGTGTTCACTGTCAGAAAGAAGGAGGAGTACAAGATGGCCCTGTACAACCTGTACCCTGGAGTATTTGAGACTGTGGAGATGCTGCCCAGCAAGGCAGGTATCTGGAGAGTGGAGTGCCTGATTGGGG |
| | AGCACCTGCATGCAGGTATGAGCACCCTGTTCCTGGTGTACAGCAACAAGTGCCAGACCCCCCTGGGCATGGCCTCTGGACACATCAGAGACTTCCAGATCACTGCCAGTGGCCAGTATGGCCAGTGGGCCCCCAAGCTGGCCAG |
| | ACTGCACTACTCAGGGAGCATCAATGCCTGGAGCACCAAGGAGCCCTTCAGCTGGATCAAGGTGGACCTGCTGGCCCCCATGATCATCCATGGTATCAAGACCCAGGGTGCCAGACAGAAGTTCAGCAGCCTGTACATCAGCCAG |
| | TTCATCATCATGTACAGCCTGGATGGCAAGAAGTGGCAGACCTACAGAGGCAACAGCACAGGCACCCTGATGGTGTTCTTTGGCAATGTAGACAGCTCTGGCATCAAGCACAACATCTTCAACCCCCCCATCATAGCTAGATACA |
| | TCAGACTGCACCCCACCCACTACAGCATCAGAAGCACCCTGAGAATGGAGCTGATGGGCTGTGACCTGAACAGCTGCAGCATGCCCCTGGGCATGGAGAGCAAGGCCATCTCAGATGCTCAGATCACTGCCAGCAGCTACTTCAC |
| | CAACATGTTTGCCACCTGGAGCCCCAGCAAGGCCAGACTGCACCTGCAGGGCAGAAGCAATGCCTGGAGACCCCAGGTGAACAACCCCAAGGAGTGGCTGCAGGTGGACTTCCAGAAGACCATGAAGGTGACAGGAGTGACCACC |
| | CAGGGGGTGAAGAGCCTGCTGACCAGCATGTATGTGAAGGAGTTCCTGATCAGCAGCAGCCAGGATGGACACCAGTGGACCCTGTTCTTCCAGAATGGAAAGGTGAAGGTGTTCCAGGGCAACCAGGACAGCTTCACCCCTGTGG |
| | TGAACAGCCTGGACCCCCCCCTGCTGACCAGATACCTGAGAATCCACCCCCAGAGCTGGGTGCACCAGATTGCCCTGAGAATGGAGGTGCTGGGCTGTGAGGCCCAGGACCTGTACTGATTTAAATCTCGAGCCGGGCGGAGTGT |
| | GTTAGTCTCTCCAGAGGGAGGCTGGTTCCCCAGGGAAGCAGAGCCTGTGTGCGGGCAGCAGCTGTGTGCGGGCCTGGGGGTTGTTAAGTGCAATTATTTTTAATAAAAGGGGCATTTGGAAAAAAAAAAAAAAGGTAGCAGTCGA |
| | CAGATGAATTCTGCAGATCTGTGGCTTCTAGCTGCCCGGGTGGCATCCCTGTGACCCCTCCCCAGTGCCTCTCCTGGCCCTGGAAGTTGCCACTCCAGTGCCCACCAGCCTTGTCCTAATAAAATTAAGTTGCATCATTTTGTCT |
| | GACTAGGTGTCCTTCTATAATATTATGGGGTGGAGGGGGGTGGTATGGAGCAAGGGGCAAGTTGGGAAGACAACCTGTAGGGCCTGCGGGGTCTATTGGGAACCAAGCTGGAGTGCAGTGGCACAATCTTGGCTCACTGCAATCT |
| | CCGCCTCCTGGGTTCAAGCGATTCTCCTGCCTCAGCCTCCCGAGTTGTTGGGATTCCAGGCATGCATGACCAGGCTCAGCTAATTTTTGTTTTTTTGGTAGAGACGGGGTTTCACCATATTGGCCAGGCTGGTCTCCAACTCCTA |
| | ATCTCAGGTGATCTACCCACCTTGGCCTCCCAAATTGCTGGGATTACAGGCGTGAACCACTGCTCCCTTCCCTGTCCTTCTGATTTTAAAAAAAAGAGAAATGTTCTGGCACCTGCACTTGCACTGGGGACAGCCTATTTTGCTA |
| | GTTTGTTTTGTTTCGTTTTGTTTTGATGGAGAGCGTATGTTGTTTAAACGCGGCCGCGTAGATAAGTAGCATGGCGGGTTAATCATTAACTACAAGGAACCCCTAGTGATGGAGTTGGCCACTCCCTCTCTGCGCGCTCGCTCGC |
| | TCACTGAGGCCGGGCGACCAAAGGTCGCCCGACGCCCGGGCTTTGCCCGGGCGGCCTCAGTGAGCGAGCGAGCGCGCAG |
| 30 | >ITR-to-ITR construct with FVIII-QQ5 nucleic acid |
| | >[L-ITR]-[ACTUS101-LSP]-[5′ UTR]-[FVIII-005(w/sp)]-[ 3′ UTR]-[PolIII_terminator]-[hGHpolyA]-[PolII_terminator-rev][R-ITR] |
| | CTGCGCGCTCGCTCGCTCACTGAGGCCGCCCGGGCAAAGCCCGGGCGTCGGGCGACCTTTGGTCGCCCGGCCTCAGTGAGCGAGCGAGCGCGCAGAGAGGGAGTGGCCAACTCCATCACTAGGGGTTCCTTGTAGTTAATGATTA |
| | ACCCGCCATGCTACTTATCTACGGCGCGCCACGCGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACT |
| | AGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACTAGTTCTAGAGCGGCCGCCAGTGTGCTGGAATTC |
| | GGCTTTTTTAGGGCTGGAAGCTACCTTTGACATCATTTCCTCTGCGAATGCATGTATAATTTCTACAGAACCTATTAGAAAGGATCACCCAGCCTCTGCTTTTGTACAACTTTCCCTTAAAAAACTGCCAATCCCACTGCTGTTT |
| | GGCCCAATAGTGAGAACTTTTTCCTGCTGCCTCTTGGTGCTTTTGCCTATGGCCCCTATTCTGCCTGCTGAAGACACTCTTGCCAGCATGGACTTAAACCCCTCCAGCTCTGACAATCCTCTTTCTCTTTTGTTTTACATGAAGG |
| | GTCTGGCAGCCAAAGCAATCACTCAAAGTTCAAACCTTATCATTTTTTGCTTTGTTCCTCTTGGCCTTGGTTTTGTACATCAGCTTTGAAAATACCATCCCAGGGTTAATGCTGGGGTTAATTTATAACTGAGAGTGCTCTAGTT |
| | CTGCAATACAGGACATGCTATAAAAATGGAAAGATGTTGCTTTCTGAGAGATCAGGTGGATTCTTGGGCATTTGCTTAAGCTTGGTACCGCCACCATGCAGATTGAGCTGAGCACCTGCTTCTTCCTGTGCCTGCTGAGATTCTG |
| | CTTCTCTGCTACCAGAAGATACTACCTGGGAGCTGTGGAGCTGAGCTGGGACTACATGCAGTCAGACCTGGGTGAACTGCCTGTGGATGCCAGATTCCCCCCCAGAGTGCCCAAGAGCTTCCCCTTCAACACCAGTGTTGTCTAC |
| | AAGAAGACCCTGTTTGTGGAGTTCACAGACCACCTGTTCAACATTGCCAAGCCCAGACCCCCCTGGATGGGCCTGCTGGGCCCCACCATCCAGGCTGAAGTGTATGATACAGTGGTGATCACCCTGAAGAACATGGCCAGCCACC |
| | CAGTGAGCCTGCATGCAGTGGGTGTGAGCTACTGGAAGGCCTCTGAAGGTGCAGAGTATGATGACCAGACCAGCCAGAGAGAGAAGGAGGATGACAAGGTGTTCCCTGGGGGCAGCCACACCTATGTGTGGCAGGTGCTGAAGGA |
| | GAATGGCCCCATGGCCAGTGATCCCCTGTGCCTGACCTACAGCTACCTGAGCCATGTTGACCTGGTGAAGGACCTGAACTCAGGTCTGATTGGAGCCCTGCTGGTGTGCAGAGAGGGCAGCCTGGCCAAGGAGAAGACCCAGACC |
| | CTGCACAAGTTCATCCTGCTGTTTGCTGTCTTTGATGAAGGCAAGAGCTGGCACAGTGAGACCAAGAACAGCCTGATGCAGGACAGAGATGCAGCCTCTGCCAGAGCCTGGCCCAAGATGCACACAGTAAATGGCTATGTGAACA |
| | GAAGCCTGCCAGGGCTGATTGGCTGCCACAGAAAGTCTGTTTACTGGCATGTGATTGGGATGGGCACCACCCCTGAAGTGCACAGCATCTTCCTGGAGGGCCACACCTTCCTGGTGAGAAACCACAGACAGGCCAGCCTGGAGAT |
| | CAGCCCCATCACCTTCCTGACAGCACAGACCCTGCTGATGGACCTGGGCCAGTTCCTGCTGTTCTGCCACATCAGCAGCCACCAGCATGATGGGATGGAGGCCTATGTGAAGGTGGACAGCTGCCCAGAAGAGCCCCAGCTGCAG |
| | ATGAAGAACAATGAGGAGGCAGAGGACTATGATGATGACCTGACTGACTCTGAGATGGATGTTGTGAGATTTGATGATGACAACAGCCCCAGCTTCATCCAGATCAGAAGTGTTGCCAAGAAGCACCCCAAGACCTGGGTGCACT |
| | ACATTGCAGCTGAGGAGGAGGACTGGGACTATGCCCCCCTGGTGCTGGCCCCAGATGACAGAAGCTACAAGAGCCAGTACCTGAACAATGGCCCCCAGAGAATTGGTAGAAAGTACAAGAAGGTGAGATTCATGGCCTACACAGA |
| | TGAGACCTTCAAGACCAGAGAGGCCATCCAGCATGAGTCAGGAATCCTGGGCCCCCTGCTGTATGGGGAGGTGGGGGATACCCTGCTGATCATCTTCAAGAACCAGGCCAGCAGACCCTACAACATCTACCCCCATGGCATCACA |
| | GATGTGAGACCCCTGTACAGCAGAAGACTGCCCAAGGGAGTGAAGCACCTGAAGGACTTCCCCATCCTGCCTGGTGAGATCTTCAAGTACAAGTGGACAGTTACTGTGGAAGATGGTCCCACCAAGTCAGACCCCAGATGCCTGA |
| | CCAGATACTACAGCAGCTTTGTGAACATGGAGAGAGACCTGGCCTCTGGGCTGATTGGGCCCCTGCTGATCTGCTACAAGGAGAGTGTGGACCAGCAGGGCAACCAGATCATGTCTGATAAGAGAAATGTGATCCTGTTCTCTGT |
| | ATTTGATGAAAACAGAAGCTGGTACCTGACTGAGAACATCCAGAGATTCCTGCCCAACCCAGCTGGAGTCCAGCTGGAGGACCCAGAGTTCCAGGCCAGCAACATCATGCACAGCATCAATGGATATGTGTTTGACAGCCTGCAG |
| | CTGTCTGTATGCCTGCATGAGGTGGCCTACTGGTACATCCTGAGCATTGGGGCCCAGACTGACTTCCTGTCTGTGTTCTTCTCAGGATACACCTTCAAGCACAAGATGGTGTATGAGGACACCCTGACCCTGTTCCCCTTCTCAG |
| | GGGAAACTGTGTTCATGAGCATGGAGAACCCTGGCCTGTGGATCCTGGGCTGCCACAACTCTGACTTCAGAAACAGAGGCATGACTGCCCTGCTGAAGGTGAGCAGCTGTGACAAGAACACTGGGGATTACTATGAGGACAGCTA |
| | TGAGGACATCTCAGCCTACCTGCTGAGCAAGAACAATGCCATTGAACCCAGAAGCTTCAGCCAGAACCCCCCTGTCCTGAAGAGACACCAGAGAGAGATCACCAGAACCACCCTGCAGTCTGATCAGGAGGAGATTGACTATGAT |
| | GATACCATCTCAGTGGAGATGAAGAAGGAGGACTTTGACATCTATGATGAAGATGAAAACCAGAGCCCCAGAAGCTTCCAGAAGAAGACCAGACACTACTTCATTGCTGCTGTGGAGAGACTGTGGGACTATGGGATGAGCAGCA |
| | GCCCCCATGTGCTGAGAAACAGAGCCCAGTCAGGCAGTGTGCCCCAGTTCAAGAAGGTGGTGTTCCAGGAGTTCACAGATGGAAGCTTCACCCAGCCCCTGTACAGAGGTGAGCTGAATGAACACCTGGGCCTGCTGGGCCCCTA |
| | CATCAGAGCTGAAGTGGAGGACAACATCATGGTGACCTTCAGAAACCAGGCCAGCAGACCCTACAGCTTCTACAGCAGCCTGATCAGCTATGAGGAGGACCAGAGACAGGGAGCAGAGCCCAGAAAGAACTTTGTCAAGCCCAAT |
| | GAAACCAAGACCTACTTCTGGAAGGTGCAGCACCACATGGCCCCCACCAAGGATGAATTTGACTGCAAGGCCTGGGCCTACTTCTCTGATGTAGACCTGGAGAAGGATGTGCACTCTGGGCTGATTGGCCCCCTGCTGGTGTGCC |
| | ACACCAACACCCTGAACCCAGCCCATGGCAGACAGGTGACTGTGCAGGAGTTTGCCCTGTTCTTCACCATCTTTGATGAAACCAAGAGCTGGTACTTCACTGAGAACATGGAGAGAAACTGCAGAGCCCCCTGCAACATCCAGAT |
| | GGAGGACCCCACCTTCAAGGAGAACTACAGATTCCATGCTATCAATGGATACATCATGGACACCCTGCCTGGGCTGGTGATGGCCCAGGACCAGAGAATCAGATGGTACCTGCTGAGCATGGGCAGCAATGAGAACATCCACAGC |
| | ATCCACTTCTCAGGACATGTCTTCACAGTAAGAAAGAAGGAGGAGTACAAGATGGCCCTGTACAACCTGTACCCAGGGGTCTTTGAAACTGTAGAGATGCTGCCCAGCAAGGCAGGCATCTGGAGAGTGGAGTGCCTGATTGGAG |
| | AGCACCTGCATGCTGGTATGAGCACCCTGTTCCTGGTGTACAGCAACAAGTGCCAGACCCCCCTGGGCATGGCCTCTGGCCACATCAGAGACTTCCAGATCACAGCCAGTGGCCAGTATGGGCAGTGGGCCCCCAAGCTGGCCAG |
| | ACTGCACTACTCTGGCAGCATCAATGCCTGGAGCACCAAGGAGCCCTTCAGCTGGATCAAGGTGGACCTGCTGGCCCCCATGATCATCCATGGGATCAAGACCCAGGGTGCCAGACAGAAGTTCAGCAGCCTGTACATCAGCCAG |
| | TTCATCATCATGTACAGCCTGGATGGCAAGAAGTGGCAGACCTACAGAGGCAACAGCACAGGTACCCTGATGGTGTTCTTTGGCAATGTGGACAGCTCAGGCATCAAGCACAACATCTTCAACCCCCCCATCATTGCCAGATACA |
| | TCAGACTGCACCCCACCCACTACAGCATCAGAAGCACCCTGAGAATGGAGCTGATGGGCTGTGACCTGAACAGCTGCAGCATGCCCCTGGGCATGGAGAGCAAGGCCATCTCTGATGCCCAGATCACAGCTAGCAGCTACTTCAC |
| | CAACATGTTTGCCACCTGGAGCCCCAGCAAGGCCAGACTGCACCTGCAGGGCAGAAGCAATGCTTGGAGACCCCAGGTGAACAACCCCAAGGAGTGGCTGCAGGTGGACTTCCAGAAGACCATGAAGGTGACAGGTGTGACCACC |
| | CAGGGGGTTAAGAGCCTGCTGACCAGCATGTATGTGAAGGAGTTCCTGATCAGCAGCAGCCAGGATGGCCACCAGTGGACCCTGTTCTTCCAGAATGGCAAGGTGAAGGTGTTCCAGGGCAACCAGGACAGCTTCACCCCTGTTG |
| | TGAACAGCCTGGACCCCCCCCTGCTGACCAGATACCTGAGAATCCACCCCCAGAGCTGGGTGCACCAGATTGCCCTGAGAATGGAGGTGCTGGGCTGTGAGGCCCAGGACCTGTACTGATTTAAATCTCGAGCCGGGCGGAGTGT |
| | GTTAGTCTCTCCAGAGGGAGGCTGGTTCCCCAGGGAAGCAGAGCCTGTGTGCGGGCAGCAGCTGTGTGCGGGCCTGGGGGTTGTTAAGTGCAATTATTTTTAATAAAAGGGGCATTTGGAAAAAAAAAAAAAAGGTAGCAGTCGA |
| | CAGATGAATTCTGCAGATCTGTGGCTTCTAGCTGCCCGGGTGGCATCCCTGTGACCCCTCCCCAGTGCCTCTCCTGGCCCTGGAAGTTGCCACTCCAGTGCCCACCAGCCTTGTCCTAATAAAATTAAGTTGCATCATTTTGTCT |
| | GACTAGGTGTCCTTCTATAATATTATGGGGTGGAGGGGGGTGGTATGGAGCAAGGGGCAAGTTGGGAAGACAACCTGTAGGGCCTGCGGGGTCTATTGGGAACCAAGCTGGAGTGCAGTGGCACAATCTTGGCTCACTGCAATCT |
| | CCGCCTCCTGGGTTCAAGCGATTCTCCTGCCTCAGCCTCCCGAGTTGTTGGGATTCCAGGCATGCATGACCAGGCTCAGCTAATTTTTGTTTTTTTGGTAGAGACGGGGTTTCACCATATTGGCCAGGCTGGTCTCCAACTCCTA |
| | ATCTCAGGTGATCTACCCACCTTGGCCTCCCAAATTGCTGGGATTACAGGCGTGAACCACTGCTCCCTTCCCTGTCCTTCTGATTTTAAAAAAAAGAGAAATGTTCTGGCACCTGCACTTGCACTGGGGACAGCCTATTTTGCTA |
| | GTTTGTTTTGTTTCGTTTTGTTTTGATGGAGAGCGTATGTTGTTTAAACGCGGCCGCGTAGATAAGTAGCATGGCGGGTTAATCATTAACTACAAGGAACCCCTAGTGATGGAGTTGGCCACTCCCTCTCTGCGCGCTCGCTCGC |
| | TCACTGAGGCCGGGCGACCAAAGGTCGCCCGACGCCCGGGCTTTGCCCGGGCGGCCTCAGTGAGCGAGCGAGCGCGCAG |
| 31 | >ITR-to-ITR construct with FVIII-QQ6 nucleic acid |
| | >[L-ITR]-[ACTUS101-LSP]-[5′ UTR]-[FVIII-QQ6(w/sp)]-[3′ UTR]-[PolIII_terminator]-[hGHpolyA]-[PolII_terminator-rev][R-ITR] |
| | CTGCGCGCTCGCTCGCTCACTGAGGCCGCCCGGGCAAAGCCCGGGCGTCGGGCGACCTTTGGTCGCCCGGCCTCAGTGAGCGAGCGAGCGCGCAGAGAGGGAGTGGCCAACTCCATCACTAGGGGTTCCTTGTAGTTAATGATTA |
| | ACCCGCCATGCTACTTATCTACGGCGCGCCACGCGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACT |
| | AGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACTAGTTCTAGAGCGGCCGCCAGTGTGCTGGAATTC |
| | GGCTTTTTTAGGGCTGGAAGCTACCTTTGACATCATTTCCTCTGCGAATGCATGTATAATTTCTACAGAACCTATTAGAAAGGATCACCCAGCCTCTGCTTTTGTACAACTTTCCCTTAAAAAACTGCCAATCCCACTGCTGTTT |
| | GGCCCAATAGTGAGAACTTTTTCCTGCTGCCTCTTGGTGCTTTTGCCTATGGCCCCTATTCTGCCTGCTGAAGACACTCTTGCCAGCATGGACTTAAACCCCTCCAGCTCTGACAATCCTCTTTCTCTTTTGTTTTACATGAAGG |
| | GTCTGGCAGCCAAAGCAATCACTCAAAGTTCAAACCTTATCATTTTTTGCTTTGTTCCTCTTGGCCTTGGTTTTGTACATCAGCTTTGAAAATACCATCCCAGGGTTAATGCTGGGGTTAATTTATAACTGAGAGTGCTCTAGTT |
| | CTGCAATACAGGACATGCTATAAAAATGGAAAGATGTTGCTTTCTGAGAGATCAGGTGGATTCTTGGGCATTTGCTTAAGCTTGGTACCGCCACCATGCAGATAGAACTGAGCACCTGCTTCTTCCTGTGCCTGCTGAGATTCTG |
| | CTTCTCAGCCACCAGAAGATACTACCTGGGGGCAGTTGAGCTGAGCTGGGACTACATGCAGAGTGATCTGGGGGAGCTGCCAGTGGATGCAAGATTCCCCCCCAGAGTGCCCAAGAGCTTCCCCTTCAACACCAGTGTGGTCTAC |
| | AAGAAGACCCTGTTTGTTGAGTTCACAGACCACCTGTTCAACATTGCTAAGCCCAGACCCCCCTGGATGGGCCTGCTGGGCCCCACCATCCAGGCAGAGGTGTATGATACAGTTGTGATCACCCTGAAGAACATGGCCAGCCACC |
| | CAGTTAGCCTGCATGCAGTTGGAGTGAGCTACTGGAAGGCCTCAGAGGGAGCTGAGTATGATGACCAGACCAGCCAGAGAGAGAAGGAGGATGATAAGGTGTTCCCAGGGGGGAGCCACACCTATGTGTGGCAGGTGCTGAAGGA |
| | GAATGGACCCATGGCCAGTGACCCCCTGTGCCTGACCTACAGCTACCTGAGCCATGTGGACCTGGTGAAGGACCTGAACTCAGGACTGATAGGTGCTCTGCTGGTGTGCAGAGAGGGCAGCCTGGCCAAGGAGAAGACCCAGACC |
| | CTGCACAAGTTCATCCTGCTGTTTGCAGTTTTTGATGAAGGCAAGAGCTGGCACTCAGAGACCAAGAACAGCCTGATGCAGGACAGAGATGCAGCTTCTGCTAGAGCCTGGCCCAAGATGCACACTGTCAATGGCTATGTAAACA |
| | GAAGCCTGCCTGGCCTGATTGGCTGCCACAGAAAGTCTGTCTACTGGCATGTTATAGGTATGGGCACCACCCCTGAGGTGCACAGCATCTTCCTGGAGGGCCACACCTTCCTGGTGAGAAACCACAGACAGGCCAGCCTGGAGAT |
| | CAGCCCCATCACCTTCCTGACTGCCCAGACCCTGCTGATGGACCTGGGCCAGTTCCTGCTGTTCTGCCACATCAGCAGCCACCAGCATGATGGCATGGAGGCCTATGTCAAGGTGGACAGCTGCCCAGAAGAGCCCCAGCTGCAG |
| | ATGAAGAACAATGAGGAGGCTGAGGACTATGATGATGACCTGACTGACAGTGAGATGGATGTAGTGAGATTTGATGATGACAACAGCCCCAGCTTCATCCAGATCAGAAGTGTAGCCAAGAAGCACCCCAAGACCTGGGTGCACT |
| | ACATTGCTGCAGAGGAGGAGGACTGGGACTATGCCCCCCTGGTGCTGGCCCCAGATGACAGAAGCTACAAGAGCCAGTACCTGAACAATGGTCCCCAGAGAATTGGCAGAAAGTACAAGAAGGTGAGATTCATGGCCTACACAGA |
| | TGAGACCTTCAAGACCAGAGAGGCCATCCAGCATGAGTCAGGCATCCTGGGCCCCCTGCTGTATGGAGAGGTGGGGGACACCCTGCTGATCATCTTCAAGAACCAGGCCAGCAGACCCTACAACATCTACCCCCATGGAATCACA |
| | GATGTGAGACCCCTGTACAGCAGAAGACTGCCCAAGGGGGTGAAGCACCTGAAGGACTTCCCCATCCTGCCTGGAGAGATCTTCAAGTACAAGTGGACTGTGACAGTAGAGGATGGACCCACCAAGTCAGACCCCAGATGCCTGA |
| | CCAGATACTACAGCAGCTTTGTAAACATGGAGAGAGACCTGGCCAGTGGCCTGATAGGCCCCCTGCTGATCTGCTACAAGGAGTCAGTGGACCAGCAGGGCAACCAGATCATGTCAGACAAGAGAAATGTGATCCTGTTCTCTGT |
| | ATTTGATGAGAACAGAAGCTGGTACCTGACTGAAAACATCCAGAGATTCCTGCCCAACCCTGCTGGAGTACAGCTGGAGGACCCTGAGTTCCAGGCCAGCAACATCATGCACAGCATCAATGGTTATGTGTTTGACAGCCTGCAG |
| | CTGTCTGTGTGCCTGCATGAAGTGGCCTACTGGTACATCCTGAGCATAGGTGCCCAGACTGACTTCCTGTCTGTGTTCTTCAGTGGCTACACCTTCAAGCACAAGATGGTGTATGAAGACACCCTGACCCTGTTCCCCTTCTCTG |
| | GGGAGACAGTATTCATGAGCATGGAGAACCCAGGACTGTGGATCCTGGGCTGCCACAACAGTGACTTCAGAAACAGAGGCATGACTGCTCTGCTGAAGGTGAGCAGCTGTGATAAGAACACAGGTGACTACTATGAGGACAGCTA |
| | TGAAGACATCAGTGCTTACCTGCTGAGCAAGAACAATGCTATAGAACCCAGAAGCTTCAGCCAGAACCCCCCAGTTCTGAAGAGACACCAGAGAGAGATCACCAGAACCACCCTGCAGTCAGACCAGGAGGAGATAGACTATGAT |
| | GATACCATCTCAGTAGAGATGAAGAAGGAGGACTTTGACATCTATGATGAGGATGAAAACCAGAGCCCCAGAAGCTTCCAGAAGAAGACCAGACACTACTTCATAGCTGCTGTAGAGAGACTGTGGGACTATGGAATGAGCAGCA |
| | GCCCCCATGTGCTGAGAAACAGAGCCCAGTCTGGGAGTGTGCCCCAGTTCAAGAAGGTGGTGTTCCAGGAGTTCACAGATGGGAGCTTCACCCAGCCCCTGTACAGAGGGGAGCTGAATGAACACCTGGGCCTGCTGGGCCCCTA |
| | CATCAGAGCAGAGGTGGAGGACAACATCATGGTGACCTTCAGAAACCAGGCCAGCAGACCCTACAGCTTCTACAGCAGCCTGATCAGCTATGAGGAGGACCAGAGACAGGGAGCTGAGCCCAGAAAGAACTTTGTTAAGCCCAAT |
| | GAAACCAAGACCTACTTCTGGAAGGTGCAGCACCACATGGCCCCCACCAAGGATGAGTTTGACTGCAAGGCCTGGGCCTACTTCTCTGATGTTGACCTGGAGAAGGATGTGCACTCTGGACTGATTGGGCCCCTGCTGGTGTGCC |
| | ACACCAACACCCTGAACCCTGCCCATGGGAGACAGGTGACAGTACAGGAGTTTGCTCTGTTCTTCACCATCTTTGATGAGACCAAGAGCTGGTACTTCACAGAAAACATGGAGAGAAACTGCAGAGCCCCCTGCAACATCCAGAT |
| | GGAGGACCCCACCTTCAAGGAGAACTACAGATTCCATGCTATCAATGGCTACATCATGGACACCCTGCCTGGCCTGGTGATGGCCCAGGACCAGAGAATCAGATGGTACCTGCTGAGCATGGGCAGCAATGAAAACATCCACAGC |
| | ATCCACTTCTCTGGTCATGTTTTCACAGTTAGAAAGAAGGAGGAGTACAAGATGGCCCTGTACAACCTGTACCCTGGGGTGTTTGAGACTGTGGAGATGCTGCCCAGCAAGGCTGGAATCTGGAGAGTGGAGTGCCTGATTGGAG |
| | AACACCTGCATGCAGGGATGAGCACCCTGTTCCTGGTGTACAGCAACAAGTGCCAGACCCCCCTGGGCATGGCCAGTGGTCACATCAGAGACTTCCAGATCACAGCCTCAGGCCAGTATGGACAGTGGGCCCCCAAGCTGGCCAG |
| | ACTGCACTACAGTGGTAGCATCAATGCCTGGAGCACCAAGGAGCCCTTCAGCTGGATCAAGGTGGACCTGCTGGCCCCCATGATCATCCATGGAATCAAGACCCAGGGGGCCAGACAGAAGTTCAGCAGCCTGTACATCAGCCAG |
| | TTCATCATCATGTACAGCCTGGATGGCAAGAAGTGGCAGACCTACAGAGGCAACAGCACTGGTACCCTGATGGTGTTCTTTGGCAATGTGGACAGCTCTGGGATCAAGCACAACATCTTCAACCCCCCCATCATAGCCAGATACA |
| | TCAGACTGCACCCCACCCACTACAGCATCAGAAGCACCCTGAGAATGGAGCTGATGGGCTGTGACCTGAACAGCTGCAGCATGCCCCTGGGCATGGAGAGCAAGGCCATCAGTGATGCTCAGATCACAGCAAGCAGCTACTTCAC |
| | CAACATGTTTGCCACCTGGAGCCCCAGCAAGGCCAGACTGCACCTGCAGGGCAGAAGCAATGCTTGGAGACCCCAGGTGAACAACCCCAAGGAGTGGCTGCAGGTGGACTTCCAGAAGACCATGAAGGTGACTGGGGTGACCACC |
| | CAGGGGGTGAAGAGCCTGCTGACCAGCATGTATGTCAAGGAGTTCCTGATCAGCAGCAGCCAGGATGGGCACCAGTGGACCCTGTTCTTCCAGAATGGGAAGGTGAAGGTGTTCCAGGGCAACCAGGACAGCTTCACCCCAGTAG |
| | TGAACAGCCTGGACCCCCCCCTGCTGACCAGATACCTGAGAATCCACCCCCAGAGCTGGGTGCACCAGATAGCCCTGAGAATGGAGGTGCTGGGCTGTGAGGCCCAGGACCTGTACTGATTTAAATCTCGAGCCGGGCGGAGTGT |
| | GTTAGTCTCTCCAGAGGGAGGCTGGTTCCCCAGGGAAGCAGAGCCTGTGTGCGGGCAGCAGCTGTGTGCGGGCCTGGGGGTTGTTAAGTGCAATTATTTTTAATAAAAGGGGCATTTGGAAAAAAAAAAAAAAGGTAGCAGTCGA |
| | CAGATGAATTCTGCAGATCTGTGGCTTCTAGCTGCCCGGGTGGCATCCCTGTGACCCCTCCCCAGTGCCTCTCCTGGCCCTGGAAGTTGCCACTCCAGTGCCCACCAGCCTTGTCCTAATAAAATTAAGTTGCATCATTTTGTCT |
| | GACTAGGTGTCCTTCTATAATATTATGGGGTGGAGGGGGGTGGTATGGAGCAAGGGGCAAGTTGGGAAGACAACCTGTAGGGCCTGCGGGGTCTATTGGGAACCAAGCTGGAGTGCAGTGGCACAATCTTGGCTCACTGCAATCT |
| | CCGCCTCCTGGGTTCAAGCGATTCTCCTGCCTCAGCCTCCCGAGTTGTTGGGATTCCAGGCATGCATGACCAGGCTCAGCTAATTTTTGTTTTTTTGGTAGAGACGGGGTTTCACCATATTGGCCAGGCTGGTCTCCAACTCCTA |
| | ATCTCAGGTGATCTACCCACCTTGGCCTCCCAAATTGCTGGGATTACAGGCGTGAACCACTGCTCCCTTCCCTGTCCTTCTGATTTTAAAAAAAAGAGAAATGTTCTGGCACCTGCACTTGCACTGGGGACAGCCTATTTTGCTA |
| | GTTTGTTTTGTTTCGTTTTGTTTTGATGGAGAGCGTATGTTGTTTAAACGCGGCCGCGTAGATAAGTAGCATGGCGGGTTAATCATTAACTACAAGGAACCCCTAGTGATGGAGTTGGCCACTCCCTCTCTGCGCGCTCGCTCGC |
| | TCACTGAGGCCGGGCGACCAAAGGTCGCCCGACGCCCGGGCTTTGCCCGGGCGGCCTCAGTGAGCGAGCGAGCGCGCAG |
| 32 | >ITR-to-ITR construct with FVIII-QQ7 nucleic acid |
| | >[L-ITR]-[ACTUS101-LSP]-[5′ UTR]-[FVIII-QQ7(w/sp)]-[3′ UTR]-[PolIII_terminator]-[hGHpolyA]-[PolII_terminator-rev][R-ITR] |
| | CTGCGCGCTCGCTCGCTCACTGAGGCCGCCCGGGCAAAGCCCGGGCGTCGGGCGACCTTTGGTCGCCCGGCCTCAGTGAGCGAGCGAGCGCGCAGAGAGGGAGTGGCCAACTCCATCACTAGGGGTTCCTTGTAGTTAATGATTA |
| | ACCCGCCATGCTACTTATCTACGGCGCGCCACGCGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACT |
| | AGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACTAGTTCTAGAGCGGCCGCCAGTGTGCTGGAATTC |
| | GGCTTTTTTAGGGCTGGAAGCTACCTTTGACATCATTTCCTCTGCGAATGCATGTATAATTTCTACAGAACCTATTAGAAAGGATCACCCAGCCTCTGCTTTTGTACAACTTTCCCTTAAAAAACTGCCAATCCCACTGCTGTTT |
| | GGCCCAATAGTGAGAACTTTTTCCTGCTGCCTCTTGGTGCTTTTGCCTATGGCCCCTATTCTGCCTGCTGAAGACACTCTTGCCAGCATGGACTTAAACCCCTCCAGCTCTGACAATCCTCTTTCTCTTTTGTTTTACATGAAGG |
| | GTCTGGCAGCCAAAGCAATCACTCAAAGTTCAAACCTTATCATTTTTTGCTTTGTTCCTCTTGGCCTTGGTTTTGTACATCAGCTTTGAAAATACCATCCCAGGGTTAATGCTGGGGTTAATTTATAACTGAGAGTGCTCTAGTT |
| | CTGCAATACAGGACATGCTATAAAAATGGAAAGATGTTGCTTTCTGAGAGATCAGGTGGATTCTTGGGCATTTGCTTAAGCTTGGTACCGCCACCATGCAGATAGAGCTGAGCACCTGCTTCTTCCTGTGCCTGCTGAGATTCTG |
| | CTTCTCAGCCACCAGAAGATACTACCTGGGTGCAGTGGAGCTGAGCTGGGACTACATGCAGAGTGACCTGGGGGAACTGCCTGTGGATGCCAGATTCCCCCCCAGAGTGCCCAAGAGCTTCCCCTTCAACACCTCTGTTGTTTAC |
| | AAGAAGACCCTGTTTGTAGAGTTCACTGACCACCTGTTCAACATAGCCAAGCCCAGACCCCCCTGGATGGGCCTGCTGGGCCCCACCATCCAGGCAGAGGTGTATGATACAGTGGTGATCACCCTGAAGAACATGGCCAGCCACC |
| | CTGTTAGCCTGCATGCAGTGGGGGTGAGCTACTGGAAGGCCAGTGAGGGAGCAGAGTATGATGATCAGACCAGCCAGAGAGAGAAGGAGGATGACAAGGTGTTCCCTGGGGGCAGCCACACCTATGTATGGCAGGTGCTGAAGGA |
| | GAATGGCCCCATGGCCTCAGATCCCCTGTGCCTGACCTACAGCTACCTGAGCCATGTGGACCTGGTGAAGGACCTGAACAGTGGTCTGATTGGGGCCCTGCTGGTGTGCAGAGAGGGCAGCCTGGCCAAGGAGAAGACCCAGACC |
| | CTGCACAAGTTCATCCTGCTGTTTGCTGTCTTTGATGAGGGCAAGAGCTGGCACTCAGAGACCAAGAACAGCCTGATGCAGGACAGAGATGCTGCCTCTGCCAGAGCCTGGCCCAAGATGCACACTGTGAATGGGTATGTGAACA |
| | GAAGCCTGCCAGGTCTGATAGGCTGCCACAGAAAGTCAGTATACTGGCATGTGATTGGCATGGGCACCACCCCAGAGGTGCACAGCATCTTCCTGGAGGGCCACACCTTCCTGGTGAGAAACCACAGACAGGCCAGCCTGGAGAT |
| | CAGCCCCATCACCTTCCTGACTGCCCAGACCCTGCTGATGGACCTGGGCCAGTTCCTGCTGTTCTGCCACATCAGCAGCCACCAGCATGATGGCATGGAGGCCTATGTTAAGGTGGACAGCTGCCCAGAGGAGCCCCAGCTGCAG |
| | ATGAAGAACAATGAAGAGGCTGAGGACTATGATGATGATCTGACAGACTCAGAAATGGATGTAGTCAGATTTGATGATGATAACAGCCCCAGCTTCATCCAGATCAGATCTGTAGCCAAGAAGCACCCCAAGACCTGGGTGCACT |
| | ACATAGCAGCAGAGGAGGAGGACTGGGACTATGCACCCCTGGTGCTGGCCCCTGATGACAGAAGCTACAAGAGCCAGTACCTGAACAATGGCCCCCAGAGAATAGGCAGAAAGTACAAGAAGGTGAGATTCATGGCCTACACTGA |
| | TGAGACCTTCAAGACCAGAGAGGCCATCCAGCATGAGTCTGGAATCCTGGGCCCCCTGCTGTATGGTGAGGTGGGTGACACCCTGCTGATCATCTTCAAGAACCAGGCCAGCAGACCCTACAACATCTACCCCCATGGCATCACT |
| | GATGTGAGACCCCTGTACAGCAGAAGACTGCCCAAGGGGGTGAAGCACCTGAAGGACTTCCCCATCCTGCCAGGTGAGATCTTCAAGTACAAGTGGACTGTGACAGTGGAGGATGGCCCCACCAAGAGTGACCCCAGATGCCTGA |
| | CCAGATACTACAGCAGCTTTGTCAACATGGAGAGAGACCTGGCCAGTGGGCTGATAGGGCCCCTGCTGATCTGCTACAAGGAGTCAGTGGACCAGCAGGGCAACCAGATCATGAGTGATAAGAGAAATGTGATCCTGTTCTCAGT |
| | TTTTGATGAAAACAGAAGCTGGTACCTGACAGAAAACATCCAGAGATTCCTGCCCAACCCAGCAGGGGTACAGCTGGAGGACCCTGAATTCCAGGCCAGCAACATCATGCACAGCATCAATGGCTATGTATTTGACAGCCTGCAG |
| | CTGTCTGTTTGCCTGCATGAGGTGGCCTACTGGTACATCCTGAGCATAGGAGCCCAGACAGACTTCCTGAGTGTGTTCTTCAGTGGCTACACCTTCAAGCACAAGATGGTGTATGAGGACACCCTGACCCTGTTCCCCTTCTCAG |
| | GGGAGACTGTTTTCATGAGCATGGAGAACCCTGGACTGTGGATCCTGGGCTGCCACAACTCTGACTTCAGAAACAGAGGCATGACTGCCCTGCTGAAGGTGAGCAGCTGTGATAAGAACACTGGAGACTACTATGAGGACAGCTA |
| | TGAGGACATCTCAGCCTACCTGCTGAGCAAGAACAATGCCATTGAACCCAGAAGCTTCAGCCAGAACCCCCCTGTACTGAAGAGACACCAGAGAGAGATCACCAGAACCACCCTGCAGAGTGACCAGGAGGAGATAGACTATGAT |
| | GATACCATCTCTGTGGAGATGAAGAAGGAGGACTTTGATATCTATGATGAGGATGAGAACCAGAGCCCCAGAAGCTTCCAGAAGAAGACCAGACACTACTTCATAGCAGCAGTGGAGAGACTGTGGGACTATGGCATGAGCAGCA |
| | GCCCCCATGTGCTGAGAAACAGAGCCCAGTCAGGCAGTGTTCCCCAGTTCAAGAAGGTGGTGTTCCAGGAGTTCACTGATGGTAGCTTCACCCAGCCCCTGTACAGAGGAGAGCTGAATGAGCACCTGGGCCTGCTGGGCCCCTA |
| | CATCAGAGCTGAGGTGGAGGACAACATCATGGTGACCTTCAGAAACCAGGCCAGCAGACCCTACAGCTTCTACAGCAGCCTGATCAGCTATGAGGAGGACCAGAGACAGGGAGCAGAGCCCAGAAAGAACTTTGTTAAGCCCAAT |
| | GAAACCAAGACCTACTTCTGGAAGGTGCAGCACCACATGGCCCCCACCAAGGATGAGTTTGATTGCAAGGCCTGGGCCTACTTCAGTGATGTAGACCTGGAGAAGGATGTGCACTCAGGCCTGATTGGACCCCTGCTGGTGTGCC |
| | ACACCAACACCCTGAACCCTGCACATGGAAGACAGGTGACAGTGCAGGAGTTTGCCCTGTTCTTCACCATCTTTGATGAGACCAAGAGCTGGTACTTCACAGAGAACATGGAGAGAAACTGCAGAGCCCCCTGCAACATCCAGAT |
| | GGAGGACCCCACCTTCAAGGAGAACTACAGATTCCATGCTATCAATGGCTACATCATGGACACCCTGCCTGGCCTGGTGATGGCCCAGGACCAGAGAATCAGATGGTACCTGCTGAGCATGGGCAGCAATGAGAACATCCACAGC |
| | ATCCACTTCAGTGGGCATGTGTTCACAGTGAGAAAGAAGGAGGAGTACAAGATGGCCCTGTACAACCTGTACCCTGGAGTGTTTGAGACTGTGGAGATGCTGCCCAGCAAGGCAGGCATCTGGAGAGTGGAGTGCCTGATAGGAG |
| | AACACCTGCATGCTGGCATGAGCACCCTGTTCCTGGTGTACAGCAACAAGTGCCAGACCCCCCTGGGCATGGCCAGTGGACACATCAGAGACTTCCAGATCACAGCCTCTGGACAGTATGGACAGTGGGCCCCCAAGCTGGCCAG |
| | ACTGCACTACTCAGGAAGCATCAATGCATGGAGCACCAAGGAGCCCTTCAGCTGGATCAAGGTGGACCTGCTGGCCCCCATGATCATCCATGGCATCAAGACCCAGGGGGCAAGACAGAAGTTCAGCAGCCTGTACATCAGCCAG |
| | TTCATCATCATGTACAGCCTGGATGGCAAGAAGTGGCAGACCTACAGAGGCAACAGCACTGGAACCCTGATGGTGTTCTTTGGGAATGTGGACAGCTCAGGGATCAAGCACAACATCTTCAACCCCCCCATCATTGCTAGATACA |
| | TCAGACTGCACCCCACCCACTACAGCATCAGAAGCACCCTGAGAATGGAGCTGATGGGCTGTGATCTGAACAGCTGCAGCATGCCCCTGGGCATGGAGAGCAAGGCCATCTCAGATGCCCAGATCACTGCCAGCAGCTACTTCAC |
| | CAACATGTTTGCCACCTGGAGCCCCAGCAAGGCCAGACTGCACCTGCAGGGCAGAAGCAATGCTTGGAGACCCCAGGTGAACAACCCCAAGGAGTGGCTGCAGGTGGACTTCCAGAAGACCATGAAGGTGACTGGAGTAACCACC |
| | CAGGGGGTGAAGAGCCTGCTGACCAGCATGTATGTGAAGGAGTTCCTGATCAGCAGCAGCCAGGATGGTCACCAGTGGACCCTGTTCTTCCAGAATGGCAAGGTGAAGGTGTTCCAGGGCAACCAGGACAGCTTCACCCCTGTTG |
| | TGAACAGCCTGGACCCCCCCCTGCTGACCAGATACCTGAGAATCCACCCCCAGAGCTGGGTGCACCAGATTGCCCTGAGAATGGAGGTGCTGGGCTGTGAGGCCCAGGACCTGTACTGATTTAAATCTCGAGCCGGGCGGAGTGT |
| | GTTAGTCTCTCCAGAGGGAGGCTGGTTCCCCAGGGAAGCAGAGCCTGTGTGCGGGCAGCAGCTGTGTGCGGGCCTGGGGGTTGTTAAGTGCAATTATTTTTAATAAAAGGGGCATTTGGAAAAAAAAAAAAAAGGTAGCAGTCGA |
| | CAGATGAATTCTGCAGATCTGTGGCTTCTAGCTGCCCGGGTGGCATCCCTGTGACCCCTCCCCAGTGCCTCTCCTGGCCCTGGAAGTTGCCACTCCAGTGCCCACCAGCCTTGTCCTAATAAAATTAAGTTGCATCATTTTGTCT |
| | GACTAGGTGTCCTTCTATAATATTATGGGGTGGAGGGGGGTGGTATGGAGCAAGGGGCAAGTTGGGAAGACAACCTGTAGGGCCTGCGGGGTCTATTGGGAACCAAGCTGGAGTGCAGTGGCACAATCTTGGCTCACTGCAATCT |
| | CCGCCTCCTGGGTTCAAGCGATTCTCCTGCCTCAGCCTCCCGAGTTGTTGGGATTCCAGGCATGCATGACCAGGCTCAGCTAATTTTTGTTTTTTTGGTAGAGACGGGGTTTCACCATATTGGCCAGGCTGGTCTCCAACTCCTA |
| | ATCTCAGGTGATCTACCCACCTTGGCCTCCCAAATTGCTGGGATTACAGGCGTGAACCACTGCTCCCTTCCCTGTCCTTCTGATTTTAAAAAAAAGAGAAATGTTCTGGCACCTGCACTTGCACTGGGGACAGCCTATTTTGCTA |
| | GTTTGTTTTGTTTCGTTTTGTTTTGATGGAGAGCGTATGTTGTTTAAACGCGGCCGCGTAGATAAGTAGCATGGCGGGTTAATCATTAACTACAAGGAACCCCTAGTGATGGAGTTGGCCACTCCCTCTCTGCGCGCTCGCTCGC |
| | TCACTGAGGCCGGGCGACCAAAGGTCGCCCGACGCCCGGGCTTTGCCCGGGCGGCCTCAGTGAGCGAGCGAGCGCGCAG |
| 33 | >ITR-to-ITR construct with FVIII-QQ8 nucleic acid |
| | >[L-ITR]-[ACTUS101-LSP]-[5′ UTR]-[FVIII-QQ8(w/sp)]-[3′ UTR]-[PolIII_terminator]-[hGHpolyA]-[PolII_terminator-rev][R-ITR] |
| | CTGCGCGCTCGCTCGCTCACTGAGGCCGCCCGGGCAAAGCCCGGGCGTCGGGCGACCTTTGGTCGCCCGGCCTCAGTGAGCGAGCGAGCGCGCAGAGAGGGAGTGGCCAACTCCATCACTAGGGGTTCCTTGTAGTTAATGATTA |
| | ACCCGCCATGCTACTTATCTACGGCGCGCCACGCGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACT |
| | AGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACTAGTTCTAGAGCGGCCGCCAGTGTGCTGGAATTC |
| | GGCTTTTTTAGGGCTGGAAGCTACCTTTGACATCATTTCCTCTGCGAATGCATGTATAATTTCTACAGAACCTATTAGAAAGGATCACCCAGCCTCTGCTTTTGTACAACTTTCCCTTAAAAAACTGCCAATCCCACTGCTGTTT |
| | GGCCCAATAGTGAGAACTTTTTCCTGCTGCCTCTTGGTGCTTTTGCCTATGGCCCCTATTCTGCCTGCTGAAGACACTCTTGCCAGCATGGACTTAAACCCCTCCAGCTCTGACAATCCTCTTTCTCTTTTGTTTTACATGAAGG |
| | GTCTGGCAGCCAAAGCAATCACTCAAAGTTCAAACCTTATCATTTTTTGCTTTGTTCCTCTTGGCCTTGGTTTTGTACATCAGCTTTGAAAATACCATCCCAGGGTTAATGCTGGGGTTAATTTATAACTGAGAGTGCTCTAGTT |
| | CTGCAATACAGGACATGCTATAAAAATGGAAAGATGTTGCTTTCTGAGAGATCAGGTGGATTCTTGGGCATTTGCTTAAGCTTGGTACCGCCACCATGCAGATAGAGCTGAGCACCTGCTTCTTCCTGTGCCTGCTGAGATTCTG |
| | CTTCTCTGCCACCAGAAGATACTACCTGGGTGCAGTGGAGCTGAGCTGGGACTACATGCAGTCAGACCTGGGAGAGCTGCCAGTGGATGCTAGATTCCCCCCCAGAGTGCCCAAGAGCTTCCCCTTCAACACCTCAGTGGTGTAC |
| | AAGAAGACCCTGTTTGTAGAGTTCACTGACCACCTGTTCAACATTGCCAAGCCCAGACCCCCCTGGATGGGCCTGCTGGGCCCCACCATCCAGGCTGAGGTGTATGATACAGTGGTAATCACCCTGAAGAACATGGCCAGCCACC |
| | CAGTGAGCCTGCATGCTGTTGGAGTTAGCTACTGGAAGGCCTCTGAGGGTGCTGAGTATGATGATCAGACCAGCCAGAGAGAGAAGGAGGATGATAAGGTGTTCCCAGGGGGAAGCCACACCTATGTATGGCAGGTGCTGAAGGA |
| | GAATGGGCCCATGGCCAGTGACCCCCTGTGCCTGACCTACAGCTACCTGAGCCATGTGGATCTGGTGAAGGACCTGAACTCAGGTCTGATAGGGGCCCTGCTGGTGTGCAGAGAGGGCAGCCTGGCCAAGGAGAAGACCCAGACC |
| | CTGCACAAGTTCATCCTGCTGTTTGCTGTATTTGATGAGGGCAAGAGCTGGCACAGTGAAACCAAGAACAGCCTGATGCAGGACAGAGATGCTGCCTCTGCCAGAGCCTGGCCCAAGATGCACACAGTGAATGGCTATGTGAACA |
| | GAAGCCTGCCTGGTCTGATAGGGTGCCACAGAAAGTCAGTGTACTGGCATGTCATTGGCATGGGCACCACCCCAGAGGTGCACAGCATCTTCCTGGAGGGCCACACCTTCCTGGTGAGAAACCACAGACAGGCCAGCCTGGAGAT |
| | CAGCCCCATCACCTTCCTGACAGCTCAGACCCTGCTGATGGACCTGGGCCAGTTCCTGCTGTTCTGCCACATCAGCAGCCACCAGCATGATGGCATGGAGGCCTATGTTAAGGTGGACAGCTGCCCAGAGGAGCCCCAGCTGCAG |
| | ATGAAGAACAATGAGGAGGCAGAGGACTATGATGATGATCTGACAGACAGTGAGATGGATGTGGTGAGATTTGATGATGACAACAGCCCCAGCTTCATCCAGATCAGATCTGTGGCCAAGAAGCACCCCAAGACCTGGGTGCACT |
| | ACATTGCAGCTGAAGAGGAGGACTGGGACTATGCCCCCCTGGTGCTGGCCCCAGATGATAGAAGCTACAAGAGCCAGTACCTGAACAATGGCCCCCAGAGAATTGGAAGAAAGTACAAGAAGGTGAGATTCATGGCCTACACTGA |
| | TGAAACCTTCAAGACCAGAGAGGCCATCCAGCATGAGTCAGGCATCCTGGGCCCCCTGCTGTATGGTGAGGTGGGTGATACCCTGCTGATCATCTTCAAGAACCAGGCCAGCAGACCCTACAACATCTACCCCCATGGCATCACT |
| | GATGTAAGACCCCTGTACAGCAGAAGACTGCCCAAGGGTGTAAAGCACCTGAAGGACTTCCCCATCCTGCCTGGGGAAATCTTCAAGTACAAGTGGACTGTGACTGTAGAGGATGGTCCCACCAAGAGTGACCCCAGATGCCTGA |
| | CCAGATACTACAGCAGCTTTGTGAACATGGAGAGAGACCTGGCCTCTGGTCTGATAGGTCCCCTGCTGATCTGCTACAAGGAGTCAGTAGACCAGCAGGGCAACCAGATCATGTCTGACAAGAGAAATGTGATCCTGTTCAGTGT |
| | GTTTGATGAGAACAGAAGCTGGTACCTGACAGAAAACATCCAGAGATTCCTGCCCAACCCAGCTGGAGTGCAGCTGGAGGACCCTGAGTTCCAGGCCAGCAACATCATGCACAGCATCAATGGGTATGTCTTTGACAGCCTGCAG |
| | CTGAGTGTGTGCCTGCATGAGGTGGCCTACTGGTACATCCTGAGCATTGGTGCTCAGACAGATTTCCTGTCTGTGTTCTTCAGTGGTTACACCTTCAAGCACAAGATGGTGTATGAAGACACCCTGACCCTGTTCCCCTTCAGTG |
| | GAGAGACAGTATTCATGAGCATGGAGAACCCTGGCCTGTGGATCCTGGGCTGCCACAACAGTGATTTCAGAAACAGAGGCATGACTGCACTGCTGAAGGTGAGCAGCTGTGACAAGAACACTGGGGATTACTATGAAGACAGCTA |
| | TGAGGACATCAGTGCCTACCTGCTGAGCAAGAACAATGCTATTGAGCCCAGAAGCTTCAGCCAGAACCCCCCAGTCCTGAAGAGACACCAGAGAGAGATCACCAGAACCACCCTGCAGAGTGACCAGGAGGAGATAGACTATGAT |
| | GACACCATCTCAGTGGAGATGAAGAAGGAGGACTTTGACATCTATGATGAGGATGAGAACCAGAGCCCCAGAAGCTTCCAGAAGAAGACCAGACACTACTTCATTGCAGCAGTAGAGAGACTGTGGGACTATGGGATGAGCAGCA |
| | GCCCCCATGTACTGAGAAACAGAGCCCAGTCTGGATCTGTGCCCCAGTTCAAGAAGGTGGTGTTCCAGGAGTTCACTGATGGCAGCTTCACCCAGCCCCTGTACAGAGGAGAGCTGAATGAGCACCTGGGCCTGCTGGGCCCCTA |
| | CATCAGAGCAGAGGTGGAGGACAACATCATGGTGACCTTCAGAAACCAGGCCAGCAGACCCTACAGCTTCTACAGCAGCCTGATCAGCTATGAGGAGGACCAGAGACAGGGTGCTGAGCCCAGAAAGAACTTTGTTAAGCCCAAT |
| | GAGACCAAGACCTACTTCTGGAAGGTGCAGCACCACATGGCCCCCACCAAGGATGAATTTGATTGCAAGGCCTGGGCCTACTTCAGTGATGTAGACCTGGAGAAGGATGTGCACAGTGGACTGATTGGGCCCCTGCTGGTGTGCC |
| | ACACCAACACCCTGAACCCAGCCCATGGCAGACAGGTGACTGTGCAGGAGTTTGCTCTGTTCTTCACCATCTTTGATGAAACCAAGAGCTGGTACTTCACAGAGAACATGGAGAGAAACTGCAGAGCCCCCTGCAACATCCAGAT |
| | GGAGGACCCCACCTTCAAGGAGAACTACAGATTCCATGCTATCAATGGCTACATCATGGACACCCTGCCAGGTCTGGTGATGGCCCAGGACCAGAGAATCAGATGGTACCTGCTGAGCATGGGCAGCAATGAGAACATCCACAGC |
| | ATCCACTTCTCTGGCCATGTCTTCACTGTGAGAAAGAAGGAGGAGTACAAGATGGCCCTGTACAACCTGTACCCAGGTGTCTTTGAGACTGTAGAGATGCTGCCCAGCAAGGCTGGCATCTGGAGAGTGGAGTGCCTGATTGGAG |
| | AGCACCTGCATGCAGGAATGAGCACCCTGTTCCTGGTGTACAGCAACAAGTGCCAGACCCCCCTGGGCATGGCCAGTGGCCACATCAGAGACTTCCAGATCACAGCCTCTGGGCAGTATGGACAGTGGGCCCCCAAGCTGGCCAG |
| | ACTGCACTACTCAGGAAGCATCAATGCCTGGAGCACCAAGGAGCCCTTCAGCTGGATCAAGGTGGACCTGCTGGCCCCCATGATCATCCATGGGATCAAGACCCAGGGGGCCAGACAGAAGTTCAGCAGCCTGTACATCAGCCAG |
| | TTCATCATCATGTACAGCCTGGATGGTAAGAAGTGGCAGACCTACAGAGGCAACAGCACAGGCACCCTGATGGTGTTCTTTGGAAATGTAGACAGCTCTGGCATCAAGCACAACATCTTCAACCCCCCCATCATAGCCAGATACA |
| | TCAGACTGCACCCCACCCACTACAGCATCAGAAGCACCCTGAGAATGGAGCTGATGGGCTGTGACCTGAACAGCTGCAGCATGCCCCTGGGCATGGAGAGCAAGGCCATCTCTGATGCTCAGATCACTGCCAGCAGCTACTTCAC |
| | CAACATGTTTGCAACCTGGAGCCCCAGCAAGGCCAGACTGCACCTGCAGGGCAGAAGCAATGCCTGGAGACCCCAGGTGAACAACCCCAAGGAGTGGCTGCAGGTGGACTTCCAGAAGACCATGAAGGTGACTGGTGTAACCACC |
| | CAGGGAGTGAAGAGCCTGCTGACCAGCATGTATGTCAAGGAGTTCCTGATCAGCAGCAGCCAGGATGGTCACCAGTGGACCCTGTTCTTCCAGAATGGAAAGGTGAAGGTGTTCCAGGGCAACCAGGACAGCTTCACCCCAGTTG |
| | TGAACAGCCTGGACCCCCCCCTGCTGACCAGATACCTGAGAATCCACCCCCAGAGCTGGGTGCACCAGATTGCCCTGAGAATGGAGGTGCTGGGCTGTGAGGCCCAGGACCTGTACTGATTTAAATCTCGAGCCGGGCGGAGTGT |
| | GTTAGTCTCTCCAGAGGGAGGCTGGTTCCCCAGGGAAGCAGAGCCTGTGTGCGGGCAGCAGCTGTGTGCGGGCCTGGGGGTTGTTAAGTGCAATTATTTTTAATAAAAGGGGCATTTGGAAAAAAAAAAAAAAGGTAGCAGTCGA |
| | CAGATGAATTCTGCAGATCTGTGGCTTCTAGCTGCCCGGGTGGCATCCCTGTGACCCCTCCCCAGTGCCTCTCCTGGCCCTGGAAGTTGCCACTCCAGTGCCCACCAGCCTTGTCCTAATAAAATTAAGTTGCATCATTTTGTCT |
| | GACTAGGTGTCCTTCTATAATATTATGGGGTGGAGGGGGGTGGTATGGAGCAAGGGGCAAGTTGGGAAGACAACCTGTAGGGCCTGCGGGGTCTATTGGGAACCAAGCTGGAGTGCAGTGGCACAATCTTGGCTCACTGCAATCT |
| | CCGCCTCCTGGGTTCAAGCGATTCTCCTGCCTCAGCCTCCCGAGTTGTTGGGATTCCAGGCATGCATGACCAGGCTCAGCTAATTTTTGTTTTTTTGGTAGAGACGGGGTTTCACCATATTGGCCAGGCTGGTCTCCAACTCCTA |
| | ATCTCAGGTGATCTACCCACCTTGGCCTCCCAAATTGCTGGGATTACAGGCGTGAACCACTGCTCCCTTCCCTGTCCTTCTGATTTTAAAAAAAAGAGAAATGTTCTGGCACCTGCACTTGCACTGGGGACAGCCTATTTTGCTA |
| | GTTTGTTTTGTTTCGTTTTGTTTTGATGGAGAGCGTATGTTGTTTAAACGCGGCCGCGTAGATAAGTAGCATGGCGGGTTAATCATTAACTACAAGGAACCCCTAGTGATGGAGTTGGCCACTCCCTCTCTGCGCGCTCGCTCGC |
| | TCACTGAGGCCGGGCGACCAAAGGTCGCCCGACGCCCGGGCTTTGCCCGGGCGGCCTCAGTGAGCGAGCGAGCGCGCAG |
| 34 | >ITR-to-ITR construct with FVIII-QQ9 nucleic acid |
| | >[L-ITR]-[ACTUS101-LSP]-[5′ UTR]-[FVIII-QQ9(w/sp)]-[3′ UTR]-[PolIII_terminator]-[hGHpolyA]-[PolII_terminator-rev][R-ITR] |
| | CTGCGCGCTCGCTCGCTCACTGAGGCCGCCCGGGCAAAGCCCGGGCGTCGGGCGACCTTTGGTCGCCCGGCCTCAGTGAGCGAGCGAGCGCGCAGAGAGGGAGTGGCCAACTCCATCACTAGGGGTTCCTTGTAGTTAATGATTA |
| | ACCCGCCATGCTACTTATCTACGGCGCGCCACGCGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACT |
| | AGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACTAGTTCTAGAGCGGCCGCCAGTGTGCTGGAATTC |
| | GGCTTTTTTAGGGCTGGAAGCTACCTTTGACATCATTTCCTCTGCGAATGCATGTATAATTTCTACAGAACCTATTAGAAAGGATCACCCAGCCTCTGCTTTTGTACAACTTTCCCTTAAAAAACTGCCAATCCCACTGCTGTTT |
| | GGCCCAATAGTGAGAACTTTTTCCTGCTGCCTCTTGGTGCTTTTGCCTATGGCCCCTATTCTGCCTGCTGAAGACACTCTTGCCAGCATGGACTTAAACCCCTCCAGCTCTGACAATCCTCTTTCTCTTTTGTTTTACATGAAGG |
| | GTCTGGCAGCCAAAGCAATCACTCAAAGTTCAAACCTTATCATTTTTTGCTTTGTTCCTCTTGGCCTTGGTTTTGTACATCAGCTTTGAAAATACCATCCCAGGGTTAATGCTGGGGTTAATTTATAACTGAGAGTGCTCTAGTT |
| | CTGCAATACAGGACATGCTATAAAAATGGAAAGATGTTGCTTTCTGAGAGATCAGGTGGATTCTTGGGCATTTGCTTAAGCTTGGTACCGCCACCATGCAGATAGAGCTGAGCACCTGCTTCTTCCTGTGCCTGCTGAGATTCTG |
| | CTTCAGTGCAACCAGAAGATACTACCTGGGTGCAGTGGAGCTGAGCTGGGACTACATGCAGTCTGATCTGGGGGAGCTGCCAGTTGATGCCAGATTCCCCCCCAGAGTGCCCAAGAGCTTCCCCTTCAACACCAGTGTTGTTTAC |
| | AAGAAGACCCTGTTTGTGGAATTCACTGACCACCTGTTCAACATAGCCAAGCCCAGACCCCCCTGGATGGGCCTGCTGGGCCCCACCATCCAGGCAGAGGTGTATGACACAGTAGTGATCACCCTGAAGAACATGGCCAGCCACC |
| | CTGTGAGCCTGCATGCAGTAGGGGTAAGCTACTGGAAGGCCTCTGAAGGTGCTGAATATGATGACCAGACCAGCCAGAGAGAGAAGGAGGATGACAAGGTGTTCCCAGGTGGAAGCCACACCTATGTCTGGCAGGTGCTGAAGGA |
| | GAATGGCCCCATGGCCAGTGACCCCCTGTGCCTGACCTACAGCTACCTGAGCCATGTGGACCTGGTGAAGGACCTGAACTCAGGTCTGATTGGAGCACTGCTGGTGTGCAGAGAGGGCAGCCTGGCCAAGGAGAAGACCCAGACC |
| | CTGCACAAGTTCATCCTGCTGTTTGCAGTGTTTGATGAAGGCAAGAGCTGGCACAGTGAGACCAAGAACAGCCTGATGCAGGACAGAGATGCAGCCAGTGCCAGAGCCTGGCCCAAGATGCACACTGTGAATGGTTATGTCAACA |
| | GAAGCCTGCCAGGGCTGATAGGCTGCCACAGAAAGAGTGTTTACTGGCATGTAATTGGGATGGGCACCACCCCAGAGGTGCACAGCATCTTCCTGGAGGGCCACACCTTCCTGGTGAGAAACCACAGACAGGCCAGCCTGGAGAT |
| | CAGCCCCATCACCTTCCTGACAGCTCAGACCCTGCTGATGGACCTGGGCCAGTTCCTGCTGTTCTGCCACATCAGCAGCCACCAGCATGATGGCATGGAGGCCTATGTAAAGGTGGACAGCTGCCCTGAGGAGCCCCAGCTGCAG |
| | ATGAAGAACAATGAGGAGGCAGAGGACTATGATGATGACCTGACTGACAGTGAGATGGATGTGGTGAGATTTGATGATGACAACAGCCCCAGCTTCATCCAGATCAGAAGTGTTGCTAAGAAGCACCCCAAGACCTGGGTGCACT |
| | ACATTGCAGCAGAGGAGGAGGACTGGGACTATGCTCCCCTGGTGCTGGCCCCTGATGACAGAAGCTACAAGAGCCAGTACCTGAACAATGGCCCCCAGAGAATTGGCAGAAAGTACAAGAAGGTGAGATTCATGGCCTACACTGA |
| | TGAGACCTTCAAGACCAGAGAGGCCATCCAGCATGAAAGTGGCATCCTGGGCCCCCTGCTGTATGGAGAGGTGGGAGACACCCTGCTGATCATCTTCAAGAACCAGGCCAGCAGACCCTACAACATCTACCCCCATGGTATCACA |
| | GATGTGAGACCCCTGTACAGCAGAAGACTGCCCAAGGGGGTGAAGCACCTGAAGGACTTCCCCATCCTGCCAGGTGAAATCTTCAAGTACAAGTGGACAGTCACAGTGGAGGATGGCCCCACCAAGAGTGACCCCAGATGCCTGA |
| | CCAGATACTACAGCAGCTTTGTGAACATGGAGAGAGACCTGGCCTCTGGCCTGATAGGCCCCCTGCTGATCTGCTACAAGGAGTCAGTGGACCAGCAGGGCAACCAGATCATGAGTGATAAGAGAAATGTGATCCTGTTCTCAGT |
| | ATTTGATGAGAACAGAAGCTGGTACCTGACTGAAAACATCCAGAGATTCCTGCCCAACCCTGCAGGGGTGCAGCTGGAGGACCCTGAATTCCAGGCCAGCAACATCATGCACAGCATCAATGGCTATGTATTTGATAGCCTGCAG |
| | CTGAGTGTGTGCCTGCATGAGGTGGCCTACTGGTACATCCTGAGCATAGGGGCCCAGACAGACTTCCTGTCTGTGTTCTTCTCAGGTTACACCTTCAAGCACAAGATGGTGTATGAAGACACCCTGACCCTGTTCCCCTTCAGTG |
| | GGGAAACAGTATTCATGAGCATGGAGAACCCTGGGCTGTGGATCCTGGGCTGCCACAACTCAGATTTCAGAAACAGAGGCATGACTGCCCTGCTGAAGGTGAGCAGCTGTGACAAGAACACTGGAGACTACTATGAGGACAGCTA |
| | TGAGGACATCAGTGCCTACCTGCTGAGCAAGAACAATGCTATAGAGCCCAGAAGCTTCAGCCAGAACCCCCCAGTCCTGAAGAGACACCAGAGAGAGATCACCAGAACCACCCTGCAGTCAGATCAGGAGGAGATAGACTATGAT |
| | GACACCATCAGTGTGGAGATGAAGAAGGAGGACTTTGACATCTATGATGAGGATGAGAACCAGAGCCCCAGAAGCTTCCAGAAGAAGACCAGACACTACTTCATAGCTGCTGTAGAGAGACTGTGGGACTATGGCATGAGCAGCA |
| | GCCCCCATGTCCTGAGAAACAGAGCCCAGTCTGGCTCTGTTCCCCAGTTCAAGAAGGTGGTGTTCCAGGAGTTCACAGATGGCAGCTTCACCCAGCCCCTGTACAGAGGGGAGCTGAATGAACACCTGGGCCTGCTGGGCCCCTA |
| | CATCAGAGCTGAAGTGGAGGACAACATCATGGTGACCTTCAGAAACCAGGCCAGCAGACCCTACAGCTTCTACAGCAGCCTGATCAGCTATGAGGAGGACCAGAGACAGGGTGCAGAACCCAGAAAGAACTTTGTTAAGCCCAAT |
| | GAGACCAAGACCTACTTCTGGAAGGTGCAGCACCACATGGCCCCCACCAAGGATGAATTTGACTGCAAGGCCTGGGCCTACTTCTCAGATGTTGACCTGGAGAAGGATGTCCACTCAGGACTGATAGGGCCCCTGCTGGTGTGCC |
| | ACACCAACACCCTGAACCCAGCCCATGGCAGACAGGTGACTGTGCAGGAGTTTGCTCTGTTCTTCACCATCTTTGATGAGACCAAGAGCTGGTACTTCACAGAGAACATGGAGAGAAACTGCAGAGCCCCCTGCAACATCCAGAT |
| | GGAGGACCCCACCTTCAAGGAGAACTACAGATTCCATGCTATCAATGGATACATCATGGACACCCTGCCAGGCCTGGTGATGGCCCAGGACCAGAGAATCAGATGGTACCTGCTGAGCATGGGCAGCAATGAGAACATCCACAGC |
| | ATCCACTTCAGTGGTCATGTGTTCACAGTAAGAAAGAAGGAGGAGTACAAGATGGCCCTGTACAACCTGTACCCAGGGGTGTTTGAAACTGTGGAAATGCTGCCCAGCAAGGCAGGGATCTGGAGAGTGGAGTGCCTGATTGGAG |
| | AACACCTGCATGCAGGCATGAGCACCCTGTTCCTGGTGTACAGCAACAAGTGCCAGACCCCCCTGGGCATGGCCAGTGGGCACATCAGAGACTTCCAGATCACAGCCTCTGGGCAGTATGGCCAGTGGGCCCCCAAGCTGGCCAG |
| | ACTGCACTACTCAGGCAGCATCAATGCCTGGAGCACCAAGGAGCCCTTCAGCTGGATCAAGGTGGACCTGCTGGCCCCCATGATCATCCATGGCATCAAGACCCAGGGAGCAAGACAGAAGTTCAGCAGCCTGTACATCAGCCAG |
| | TTCATCATCATGTACAGCCTGGATGGGAAGAAGTGGCAGACCTACAGAGGCAACAGCACTGGGACCCTGATGGTGTTCTTTGGCAATGTAGACAGCTCTGGCATCAAGCACAACATCTTCAACCCCCCCATCATTGCAAGATACA |
| | TCAGACTGCACCCCACCCACTACAGCATCAGAAGCACCCTGAGAATGGAGCTGATGGGCTGTGACCTGAACAGCTGCAGCATGCCCCTGGGCATGGAGAGCAAGGCCATCAGTGATGCCCAGATCACTGCTAGCAGCTACTTCAC |
| | CAACATGTTTGCCACCTGGAGCCCCAGCAAGGCCAGACTGCACCTGCAGGGCAGAAGCAATGCATGGAGACCCCAGGTGAACAACCCCAAGGAGTGGCTGCAGGTGGACTTCCAGAAGACCATGAAGGTGACAGGGGTTACCACC |
| | CAGGGTGTGAAGAGCCTGCTGACCAGCATGTATGTGAAGGAGTTCCTGATCAGCAGCAGCCAGGATGGCCACCAGTGGACCCTGTTCTTCCAGAATGGGAAGGTGAAGGTGTTCCAGGGCAACCAGGACAGCTTCACCCCTGTGG |
| | TGAACAGCCTGGACCCCCCCCTGCTGACCAGATACCTGAGAATCCACCCCCAGAGCTGGGTGCACCAGATTGCCCTGAGAATGGAGGTGCTGGGCTGTGAGGCCCAGGACCTGTACTGATTTAAATCTCGAGCCGGGCGGAGTGT |
| | GTTAGTCTCTCCAGAGGGAGGCTGGTTCCCCAGGGAAGCAGAGCCTGTGTGCGGGCAGCAGCTGTGTGCGGGCCTGGGGGTTGTTAAGTGCAATTATTTTTAATAAAAGGGGCATTTGGAAAAAAAAAAAAAAGGTAGCAGTCGA |
| | CAGATGAATTCTGCAGATCTGTGGCTTCTAGCTGCCCGGGTGGCATCCCTGTGACCCCTCCCCAGTGCCTCTCCTGGCCCTGGAAGTTGCCACTCCAGTGCCCACCAGCCTTGTCCTAATAAAATTAAGTTGCATCATTTTGTCT |
| | GACTAGGTGTCCTTCTATAATATTATGGGGTGGAGGGGGGTGGTATGGAGCAAGGGGCAAGTTGGGAAGACAACCTGTAGGGCCTGCGGGGTCTATTGGGAACCAAGCTGGAGTGCAGTGGCACAATCTTGGCTCACTGCAATCT |
| | CCGCCTCCTGGGTTCAAGCGATTCTCCTGCCTCAGCCTCCCGAGTTGTTGGGATTCCAGGCATGCATGACCAGGCTCAGCTAATTTTTGTTTTTTTGGTAGAGACGGGGTTTCACCATATTGGCCAGGCTGGTCTCCAACTCCTA |
| | ATCTCAGGTGATCTACCCACCTTGGCCTCCCAAATTGCTGGGATTACAGGCGTGAACCACTGCTCCCTTCCCTGTCCTTCTGATTTTAAAAAAAAGAGAAATGTTCTGGCACCTGCACTTGCACTGGGGACAGCCTATTTTGCTA |
| | GTTTGTTTTGTTTCGTTTTGTTTTGATGGAGAGCGTATGTTGTTTAAACGCGGCCGCGTAGATAAGTAGCATGGCGGGTTAATCATTAACTACAAGGAACCCCTAGTGATGGAGTTGGCCACTCCCTCTCTGCGCGCTCGCTCGC |
| | TCACTGAGGCCGGGCGACCAAAGGTCGCCCGACGCCCGGGCTTTGCCCGGGCGGCCTCAGTGAGCGAGCGAGCGCGCAG |
| 35 | >ITR-to-ITR construct with FVIII-QQ10 nucleic acid |
| | >[L-ITR]-[ACTUS101-LSP]-[5′ UTR]-[FVIII-QQ10 (w/sp)]-[3′ UTR]-[PolIII_terminator]-[hGHpolyA]-[PolII_terminator-rev][R-ITR] |
| | CTGCGCGCTCGCTCGCTCACTGAGGCCGCCCGGGCAAAGCCCGGGCGTCGGGCGACCTTTGGTCGCCCGGCCTCAGTGAGCGAGCGAGCGCGCAGAGAGGGAGTGGCCAACTCCATCACTAGGGGTTCCTTGTAGTTAATGATTA |
| | ACCCGCCATGCTACTTATCTACGGCGCGCCACGCGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACT |
| | AGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACTAGTTCTAGAGCGGCCGCCAGTGTGCTGGAATTC |
| | GGCTTTTTTAGGGCTGGAAGCTACCTTTGACATCATTTCCTCTGCGAATGCATGTATAATTTCTACAGAACCTATTAGAAAGGATCACCCAGCCTCTGCTTTTGTACAACTTTCCCTTAAAAAACTGCCAATCCCACTGCTGTTT |
| | GGCCCAATAGTGAGAACTTTTTCCTGCTGCCTCTTGGTGCTTTTGCCTATGGCCCCTATTCTGCCTGCTGAAGACACTCTTGCCAGCATGGACTTAAACCCCTCCAGCTCTGACAATCCTCTTTCTCTTTTGTTTTACATGAAGG |
| | GTCTGGCAGCCAAAGCAATCACTCAAAGTTCAAACCTTATCATTTTTTGCTTTGTTCCTCTTGGCCTTGGTTTTGTACATCAGCTTTGAAAATACCATCCCAGGGTTAATGCTGGGGTTAATTTATAACTGAGAGTGCTCTAGTT |
| | CTGCAATACAGGACATGCTATAAAAATGGAAAGATGTTGCTTTCTGAGAGATCAGGTGGATTCTTGGGCATTTGCTTAAGCTTGGTACCGCCACCATGCAGATAGAACTGAGCACCTGCTTCTTCCTGTGCCTGCTGAGATTCTG |
| | CTTCTCTGCCACCAGAAGATACTACCTGGGGGCTGTGGAGCTGAGCTGGGACTACATGCAGAGTGACCTGGGAGAGCTGCCAGTTGATGCAAGATTCCCCCCCAGAGTGCCCAAGAGCTTCCCCTTCAACACCTCTGTGGTGTAC |
| | AAGAAGACCCTGTTTGTGGAGTTCACTGATCACCTGTTCAACATAGCCAAGCCCAGACCCCCCTGGATGGGCCTGCTGGGCCCCACCATCCAGGCTGAAGTGTATGACACTGTAGTGATCACCCTGAAGAACATGGCCAGCCACC |
| | CTGTGAGCCTGCATGCTGTGGGAGTGAGCTACTGGAAGGCCTCAGAGGGAGCAGAGTATGATGACCAGACCAGCCAGAGAGAGAAGGAGGATGACAAGGTGTTCCCAGGAGGCAGCCACACCTATGTTTGGCAGGTGCTGAAGGA |
| | GAATGGGCCCATGGCCTCAGACCCCCTGTGCCTGACCTACAGCTACCTGAGCCATGTAGACCTGGTGAAGGACCTGAACTCTGGCCTGATAGGAGCCCTGCTGGTGTGCAGAGAGGGCAGCCTGGCCAAGGAGAAGACCCAGACC |
| | CTGCACAAGTTCATCCTGCTGTTTGCAGTGTTTGATGAGGGCAAGAGCTGGCACTCAGAGACCAAGAACAGCCTGATGCAGGACAGAGATGCAGCCAGTGCTAGAGCCTGGCCCAAGATGCACACAGTGAATGGGTATGTTAACA |
| | GAAGCCTGCCAGGCCTGATAGGATGCCACAGAAAGTCTGTGTACTGGCATGTGATTGGTATGGGCACCACCCCAGAGGTGCACAGCATCTTCCTGGAGGGCCACACCTTCCTGGTGAGAAACCACAGACAGGCCAGCCTGGAGAT |
| | CAGCCCCATCACCTTCCTGACAGCCCAGACCCTGCTGATGGACCTGGGCCAGTTCCTGCTGTTCTGCCACATCAGCAGCCACCAGCATGATGGCATGGAGGCCTATGTTAAGGTGGACAGCTGCCCAGAGGAGCCCCAGCTGCAG |
| | ATGAAGAACAATGAAGAGGCAGAGGACTATGATGATGACCTGACAGATTCAGAGATGGATGTGGTGAGATTTGATGATGACAACAGCCCCAGCTTCATCCAGATCAGAAGTGTGGCCAAGAAGCACCCCAAGACCTGGGTGCACT |
| | ACATTGCTGCTGAAGAGGAGGACTGGGACTATGCCCCCCTGGTGCTGGCCCCAGATGACAGAAGCTACAAGAGCCAGTACCTGAACAATGGGCCCCAGAGAATTGGAAGAAAGTACAAGAAGGTGAGATTCATGGCCTACACAGA |
| | TGAAACCTTCAAGACCAGAGAGGCCATCCAGCATGAGAGTGGGATCCTGGGCCCCCTGCTGTATGGGGAGGTGGGTGATACCCTGCTGATCATCTTCAAGAACCAGGCCAGCAGACCCTACAACATCTACCCCCATGGCATCACA |
| | GATGTGAGACCCCTGTACAGCAGAAGACTGCCCAAGGGAGTTAAGCACCTGAAGGACTTCCCCATCCTGCCAGGAGAGATCTTCAAGTACAAGTGGACAGTCACAGTGGAGGATGGGCCCACCAAGTCTGACCCCAGATGCCTGA |
| | CCAGATACTACAGCAGCTTTGTAAACATGGAGAGAGACCTGGCCTCAGGTCTGATAGGCCCCCTGCTGATCTGCTACAAGGAGAGTGTTGATCAGCAGGGCAACCAGATCATGTCTGACAAGAGAAATGTGATCCTGTTCTCAGT |
| | GTTTGATGAAAACAGAAGCTGGTACCTGACTGAGAACATCCAGAGATTCCTGCCCAACCCAGCTGGGGTGCAGCTGGAGGACCCTGAGTTCCAGGCCAGCAACATCATGCACAGCATCAATGGATATGTGTTTGACAGCCTGCAG |
| | CTGTCAGTGTGCCTGCATGAGGTGGCCTACTGGTACATCCTGAGCATAGGAGCTCAGACAGACTTCCTGAGTGTATTCTTCTCAGGGTACACCTTCAAGCACAAGATGGTGTATGAGGACACCCTGACCCTGTTCCCCTTCTCAG |
| | GTGAGACAGTGTTCATGAGCATGGAGAACCCAGGGCTGTGGATCCTGGGCTGCCACAACTCTGATTTCAGAAACAGAGGCATGACTGCCCTGCTGAAGGTGAGCAGCTGTGACAAGAACACAGGGGACTACTATGAGGACAGCTA |
| | TGAGGACATCTCTGCCTACCTGCTGAGCAAGAACAATGCTATTGAGCCCAGAAGCTTCAGCCAGAACCCCCCTGTACTGAAGAGACACCAGAGAGAGATCACCAGAACCACCCTGCAGAGTGATCAGGAGGAGATAGATTATGAT |
| | GACACCATCTCAGTGGAGATGAAGAAGGAGGACTTTGATATCTATGATGAGGATGAAAACCAGAGCCCCAGAAGCTTCCAGAAGAAGACCAGACACTACTTCATTGCAGCTGTAGAGAGACTGTGGGACTATGGTATGAGCAGCA |
| | GCCCCCATGTCCTGAGAAACAGAGCCCAGTCTGGATCAGTCCCCCAGTTCAAGAAGGTGGTGTTCCAGGAGTTCACAGATGGTAGCTTCACCCAGCCCCTGTACAGAGGAGAGCTGAATGAGCACCTGGGCCTGCTGGGCCCCTA |
| | CATCAGAGCAGAGGTGGAGGACAACATCATGGTGACCTTCAGAAACCAGGCCAGCAGACCCTACAGCTTCTACAGCAGCCTGATCAGCTATGAAGAGGACCAGAGACAGGGAGCAGAGCCCAGAAAGAACTTTGTGAAGCCCAAT |
| | GAGACCAAGACCTACTTCTGGAAGGTGCAGCACCACATGGCCCCCACCAAGGATGAGTTTGATTGCAAGGCCTGGGCCTACTTCAGTGATGTTGACCTGGAGAAGGATGTTCACAGTGGCCTGATAGGTCCCCTGCTGGTGTGCC |
| | ACACCAACACCCTGAACCCAGCCCATGGCAGACAGGTGACTGTCCAGGAGTTTGCCCTGTTCTTCACCATCTTTGATGAGACCAAGAGCTGGTACTTCACTGAAAACATGGAGAGAAACTGCAGAGCCCCCTGCAACATCCAGAT |
| | GGAGGACCCCACCTTCAAGGAGAACTACAGATTCCATGCCATCAATGGCTACATCATGGACACCCTGCCTGGACTGGTGATGGCCCAGGACCAGAGAATCAGATGGTACCTGCTGAGCATGGGCAGCAATGAGAACATCCACAGC |
| | ATCCACTTCTCAGGCCATGTATTCACTGTGAGAAAGAAGGAGGAGTACAAGATGGCCCTGTACAACCTGTACCCAGGTGTGTTTGAGACAGTGGAGATGCTGCCCAGCAAGGCAGGTATCTGGAGAGTGGAGTGCCTGATAGGAG |
| | AACACCTGCATGCTGGCATGAGCACCCTGTTCCTGGTGTACAGCAACAAGTGCCAGACCCCCCTGGGCATGGCCAGTGGCCACATCAGAGACTTCCAGATCACAGCCTCAGGCCAGTATGGGCAGTGGGCCCCCAAGCTGGCCAG |
| | ACTGCACTACTCTGGCAGCATCAATGCATGGAGCACCAAGGAGCCCTTCAGCTGGATCAAGGTGGACCTGCTGGCCCCCATGATCATCCATGGAATCAAGACCCAGGGTGCTAGACAGAAGTTCAGCAGCCTGTACATCAGCCAG |
| | TTCATCATCATGTACAGCCTGGATGGCAAGAAGTGGCAGACCTACAGAGGCAACAGCACTGGCACCCTGATGGTGTTCTTTGGCAATGTTGACAGCTCTGGCATCAAGCACAACATCTTCAACCCCCCCATCATAGCAAGATACA |
| | TCAGACTGCACCCCACCCACTACAGCATCAGAAGCACCCTGAGAATGGAGCTGATGGGCTGTGATCTGAACAGCTGCAGCATGCCCCTGGGCATGGAGAGCAAGGCCATCAGTGATGCTCAGATCACTGCCAGCAGCTACTTCAC |
| | CAACATGTTTGCAACCTGGAGCCCCAGCAAGGCCAGACTGCACCTGCAGGGCAGAAGCAATGCCTGGAGACCCCAGGTGAACAACCCCAAGGAGTGGCTGCAGGTGGACTTCCAGAAGACCATGAAGGTGACAGGTGTTACCACC |
| | CAGGGTGTCAAGAGCCTGCTGACCAGCATGTATGTAAAGGAGTTCCTGATCAGCAGCAGCCAGGATGGCCACCAGTGGACCCTGTTCTTCCAGAATGGAAAGGTGAAGGTGTTCCAGGGCAACCAGGACAGCTTCACCCCTGTGG |
| | TGAACAGCCTGGACCCCCCCCTGCTGACCAGATACCTGAGAATCCACCCCCAGAGCTGGGTGCACCAGATAGCCCTGAGAATGGAGGTGCTGGGCTGTGAAGCCCAGGACCTGTACTGATTTAAATCTCGAGCCGGGCGGAGTGT |
| | GTTAGTCTCTCCAGAGGGAGGCTGGTTCCCCAGGGAAGCAGAGCCTGTGTGCGGGCAGCAGCTGTGTGCGGGCCTGGGGGTTGTTAAGTGCAATTATTTTTAATAAAAGGGGCATTTGGAAAAAAAAAAAAAAGGTAGCAGTCGA |
| | CAGATGAATTCTGCAGATCTGTGGCTTCTAGCTGCCCGGGTGGCATCCCTGTGACCCCTCCCCAGTGCCTCTCCTGGCCCTGGAAGTTGCCACTCCAGTGCCCACCAGCCTTGTCCTAATAAAATTAAGTTGCATCATTTTGTCT |
| | GACTAGGTGTCCTTCTATAATATTATGGGGTGGAGGGGGGTGGTATGGAGCAAGGGGCAAGTTGGGAAGACAACCTGTAGGGCCTGCGGGGTCTATTGGGAACCAAGCTGGAGTGCAGTGGCACAATCTTGGCTCACTGCAATCT |
| | CCGCCTCCTGGGTTCAAGCGATTCTCCTGCCTCAGCCTCCCGAGTTGTTGGGATTCCAGGCATGCATGACCAGGCTCAGCTAATTTTTGTTTTTTTGGTAGAGACGGGGTTTCACCATATTGGCCAGGCTGGTCTCCAACTCCTA |
| | ATCTCAGGTGATCTACCCACCTTGGCCTCCCAAATTGCTGGGATTACAGGCGTGAACCACTGCTCCCTTCCCTGTCCTTCTGATTTTAAAAAAAAGAGAAATGTTCTGGCACCTGCACTTGCACTGGGGACAGCCTATTTTGCTA |
| | GTTTGTTTTGTTTCGTTTTGTTTTGATGGAGAGCGTATGTTGTTTAAACGCGGCCGCGTAGATAAGTAGCATGGCGGGTTAATCATTAACTACAAGGAACCCCTAGTGATGGAGTTGGCCACTCCCTCTCTGCGCGCTCGCTCGC |
| | TCACTGAGGCCGGGCGACCAAAGGTCGCCCGACGCCCGGGCTTTGCCCGGGCGGCCTCAGTGAGCGAGCGAGCGCGCAG |
| 36 | >ITR-to-ITR construct with FVIII-QQ11 nucleic acid |
| | >[L-ITR]-[ACTUS101 -LSP]-[5′ UTR]-[FVIII-QQ11(w/sp)]-[3′ UTR]-[PolIII_terminator]-[hGHpolyA]-[PolII_terminator-rev][R-ITR] |
| | CTGCGCGCTCGCTCGCTCACTGAGGCCGCCCGGGCAAAGCCCGGGCGTCGGGCGACCTTTGGTCGCCCGGCCTCAGTGAGCGAGCGAGCGCGCAGAGAGGGAGTGGCCAACTCCATCACTAGGGGTTCCTTGTAGTTAATGATTA |
| | ACCCGCCATGCTACTTATCTACGGCGCGCCACGCGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACT |
| | AGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACTAGTTCTAGAGCGGCCGCCAGTGTGCTGGAATTC |
| | GGCTTTTTTAGGGCTGGAAGCTACCTTTGACATCATTTCCTCTGCGAATGCATGTATAATTTCTACAGAACCTATTAGAAAGGATCACCCAGCCTCTGCTTTTGTACAACTTTCCCTTAAAAAACTGCCAATCCCACTGCTGTTT |
| | GGCCCAATAGTGAGAACTTTTTCCTGCTGCCTCTTGGTGCTTTTGCCTATGGCCCCTATTCTGCCTGCTGAAGACACTCTTGCCAGCATGGACTTAAACCCCTCCAGCTCTGACAATCCTCTTTCTCTTTTGTTTTACATGAAGG |
| | GTCTGGCAGCCAAAGCAATCACTCAAAGTTCAAACCTTATCATTTTTTGCTTTGTTCCTCTTGGCCTTGGTTTTGTACATCAGCTTTGAAAATACCATCCCAGGGTTAATGCTGGGGTTAATTTATAACTGAGAGTGCTCTAGTT |
| | CTGCAATACAGGACATGCTATAAAAATGGAAAGATGTTGCTTTCTGAGAGATCAGGTGGATTCTTGGGCATTTGCTTAAGCTTGGTACCGCCACCATGCAGATAGAGCTGAGCACCTGCTTCTTCCTGTGCCTGCTGAGATTCTG |
| | CTTCTCTGCCACCAGAAGATACTACCTGGGTGCTGTTGAGCTGAGCTGGGACTACATGCAGTCTGATCTGGGTGAGCTGCCTGTTGATGCCAGATTCCCCCCCAGAGTGCCCAAGAGCTTCCCCTTCAACACCTCAGTTGTGTAC |
| | AAGAAGACCCTGTTTGTTGAGTTCACAGACCACCTGTTCAACATTGCTAAGCCCAGACCCCCCTGGATGGGCCTGCTGGGCCCCACCATCCAGGCAGAGGTGTATGATACAGTTGTGATCACCCTGAAGAACATGGCCAGCCACC |
| | CAGTGAGCCTGCATGCAGTAGGTGTGAGCTACTGGAAGGCCAGTGAGGGGGCTGAATATGATGATCAGACCAGCCAGAGAGAGAAGGAGGATGACAAGGTGTTCCCAGGTGGTAGCCACACCTATGTATGGCAGGTGCTGAAGGA |
| | GAATGGCCCCATGGCCAGTGACCCCCTGTGCCTGACCTACAGCTACCTGAGCCATGTGGACCTGGTGAAGGACCTGAACTCTGGACTGATTGGAGCACTGCTGGTGTGCAGAGAGGGCAGCCTGGCCAAGGAGAAGACCCAGACC |
| | CTGCACAAGTTCATCCTGCTGTTTGCAGTGTTTGATGAGGGCAAGAGCTGGCACTCAGAGACCAAGAACAGCCTGATGCAGGACAGAGATGCAGCTTCTGCCAGAGCCTGGCCCAAGATGCACACTGTCAATGGCTATGTGAACA |
| | GAAGCCTGCCAGGCCTGATTGGTTGCCACAGAAAGAGTGTGTACTGGCATGTTATAGGGATGGGCACCACCCCTGAGGTGCACAGCATCTTCCTGGAGGGCCACACCTTCCTGGTGAGAAACCACAGACAGGCCAGCCTGGAGAT |
| | CAGCCCCATCACCTTCCTGACAGCACAGACCCTGCTGATGGACCTGGGCCAGTTCCTGCTGTTCTGCCACATCAGCAGCCACCAGCATGATGGCATGGAGGCCTATGTTAAGGTGGACAGCTGCCCTGAGGAGCCCCAGCTGCAG |
| | ATGAAGAACAATGAGGAGGCTGAGGACTATGATGATGACCTGACAGACTCAGAGATGGATGTGGTGAGATTTGATGATGATAACAGCCCCAGCTTCATCCAGATCAGATCAGTGGCCAAGAAGCACCCCAAGACCTGGGTGCACT |
| | ACATAGCTGCTGAAGAGGAGGACTGGGACTATGCCCCCCTGGTGCTGGCCCCAGATGACAGAAGCTACAAGAGCCAGTACCTGAACAATGGGCCCCAGAGAATTGGCAGAAAGTACAAGAAGGTGAGATTCATGGCCTACACTGA |
| | TGAGACCTTCAAGACCAGAGAGGCCATCCAGCATGAGTCTGGAATCCTGGGCCCCCTGCTGTATGGAGAGGTGGGTGACACCCTGCTGATCATCTTCAAGAACCAGGCCAGCAGACCCTACAACATCTACCCCCATGGCATCACT |
| | GATGTTAGACCCCTGTACAGCAGAAGACTGCCCAAGGGTGTGAAGCACCTGAAGGACTTCCCCATCCTGCCTGGGGAGATCTTCAAGTACAAGTGGACAGTCACTGTAGAGGATGGCCCCACCAAGAGTGACCCCAGATGCCTGA |
| | CCAGATACTACAGCAGCTTTGTGAACATGGAGAGAGACCTGGCCTCTGGACTGATTGGACCCCTGCTGATCTGCTACAAGGAGAGTGTGGACCAGCAGGGCAACCAGATCATGTCAGATAAGAGAAATGTCATCCTGTTCTCAGT |
| | ATTTGATGAGAACAGAAGCTGGTACCTGACTGAGAACATCCAGAGATTCCTGCCCAACCCAGCTGGGGTGCAGCTGGAGGACCCTGAATTCCAGGCCAGCAACATCATGCACAGCATCAATGGCTATGTATTTGATAGCCTGCAG |
| | CTGTCTGTGTGCCTGCATGAGGTGGCCTACTGGTACATCCTGAGCATTGGGGCCCAGACTGATTTCCTGTCTGTCTTCTTCTCTGGGTACACCTTCAAGCACAAGATGGTGTATGAGGACACCCTGACCCTGTTCCCCTTCTCAG |
| | GAGAGACAGTGTTCATGAGCATGGAGAACCCAGGCCTGTGGATCCTGGGCTGCCACAACTCAGACTTCAGAAACAGAGGCATGACTGCCCTGCTGAAGGTGAGCAGCTGTGACAAGAACACAGGTGACTACTATGAGGACAGCTA |
| | TGAAGACATCTCTGCCTACCTGCTGAGCAAGAACAATGCTATAGAACCCAGAAGCTTCAGCCAGAACCCCCCTGTGCTGAAGAGACACCAGAGAGAGATCACCAGAACCACCCTGCAGTCAGATCAGGAGGAGATAGATTATGAT |
| | GATACCATCTCAGTAGAAATGAAGAAGGAGGACTTTGACATCTATGATGAAGATGAAAACCAGAGCCCCAGAAGCTTCCAGAAGAAGACCAGACACTACTTCATAGCTGCTGTAGAGAGACTGTGGGACTATGGCATGAGCAGCA |
| | GCCCCCATGTACTGAGAAACAGAGCCCAGAGTGGCTCAGTGCCCCAGTTCAAGAAGGTGGTGTTCCAGGAGTTCACTGATGGCAGCTTCACCCAGCCCCTGTACAGAGGTGAACTGAATGAGCACCTGGGCCTGCTGGGCCCCTA |
| | CATCAGAGCAGAGGTGGAGGACAACATCATGGTGACCTTCAGAAACCAGGCCAGCAGACCCTACAGCTTCTACAGCAGCCTGATCAGCTATGAAGAGGACCAGAGACAGGGTGCTGAGCCCAGAAAGAACTTTGTGAAGCCCAAT |
| | GAGACCAAGACCTACTTCTGGAAGGTGCAGCACCACATGGCCCCCACCAAGGATGAGTTTGACTGCAAGGCCTGGGCCTACTTCAGTGATGTAGACCTGGAGAAGGATGTGCACAGTGGGCTGATAGGTCCCCTGCTGGTGTGCC |
| | ACACCAACACCCTGAACCCAGCACATGGTAGACAGGTGACAGTTCAGGAGTTTGCCCTGTTCTTCACCATCTTTGATGAAACCAAGAGCTGGTACTTCACTGAGAACATGGAGAGAAACTGCAGAGCCCCCTGCAACATCCAGAT |
| | GGAGGACCCCACCTTCAAGGAGAACTACAGATTCCATGCCATCAATGGCTACATCATGGACACCCTGCCAGGCCTGGTGATGGCCCAGGACCAGAGAATCAGATGGTACCTGCTGAGCATGGGCAGCAATGAGAACATCCACAGC |
| | ATCCACTTCTCAGGACATGTGTTCACTGTGAGAAAGAAGGAGGAGTACAAGATGGCCCTGTACAACCTGTACCCAGGGGTATTTGAAACAGTAGAGATGCTGCCCAGCAAGGCAGGCATCTGGAGAGTGGAGTGCCTGATTGGAG |
| | AACACCTGCATGCAGGTATGAGCACCCTGTTCCTGGTGTACAGCAACAAGTGCCAGACCCCCCTGGGCATGGCCTCAGGCCACATCAGAGACTTCCAGATCACAGCTTCTGGCCAGTATGGGCAGTGGGCCCCCAAGCTGGCCAG |
| | ACTGCACTACTCAGGTAGCATCAATGCTTGGAGCACCAAGGAGCCCTTCAGCTGGATCAAGGTGGACCTGCTGGCCCCCATGATCATCCATGGGATCAAGACCCAGGGAGCCAGACAGAAGTTCAGCAGCCTGTACATCAGCCAG |
| | TTCATCATCATGTACAGCCTGGATGGTAAGAAGTGGCAGACCTACAGAGGCAACAGCACTGGCACCCTGATGGTGTTCTTTGGAAATGTTGATAGCTCAGGGATCAAGCACAACATCTTCAACCCCCCCATCATAGCCAGATACA |
| | TCAGACTGCACCCCACCCACTACAGCATCAGAAGCACCCTGAGAATGGAGCTGATGGGCTGTGATCTGAACAGCTGCAGCATGCCCCTGGGCATGGAGAGCAAGGCCATCTCTGATGCACAGATCACAGCAAGCAGCTACTTCAC |
| | CAACATGTTTGCCACCTGGAGCCCCAGCAAGGCCAGACTGCACCTGCAGGGCAGAAGCAATGCTTGGAGACCCCAGGTGAACAACCCCAAGGAGTGGCTGCAGGTGGACTTCCAGAAGACCATGAAGGTGACAGGGGTGACCACC |
| | CAGGGGGTGAAGAGCCTGCTGACCAGCATGTATGTCAAGGAGTTCCTGATCAGCAGCAGCCAGGATGGCCACCAGTGGACCCTGTTCTTCCAGAATGGGAAGGTGAAGGTGTTCCAGGGCAACCAGGACAGCTTCACCCCAGTAG |
| | TGAACAGCCTGGACCCCCCCCTGCTGACCAGATACCTGAGAATCCACCCCCAGAGCTGGGTGCACCAGATTGCTCTGAGAATGGAGGTGCTGGGCTGTGAGGCCCAGGACCTGTACTGATTTAAATCTCGAGCCGGGCGGAGTGT |
| | GTTAGTCTCTCCAGAGGGAGGCTGGTTCCCCAGGGAAGCAGAGCCTGTGTGCGGGCAGCAGCTGTGTGCGGGCCTGGGGGTTGTTAAGTGCAATTATTTTTAATAAAAGGGGCATTTGGAAAAAAAAAAAAAAGGTAGCAGTCGA |
| | CAGATGAATTCTGCAGATCTGTGGCTTCTAGCTGCCCGGGTGGCATCCCTGTGACCCCTCCCCAGTGCCTCTCCTGGCCCTGGAAGTTGCCACTCCAGTGCCCACCAGCCTTGTCCTAATAAAATTAAGTTGCATCATTTTGTCT |
| | GACTAGGTGTCCTTCTATAATATTATGGGGTGGAGGGGGGTGGTATGGAGCAAGGGGCAAGTTGGGAAGACAACCTGTAGGGCCTGCGGGGTCTATTGGGAACCAAGCTGGAGTGCAGTGGCACAATCTTGGCTCACTGCAATCT |
| | CCGCCTCCTGGGTTCAAGCGATTCTCCTGCCTCAGCCTCCCGAGTTGTTGGGATTCCAGGCATGCATGACCAGGCTCAGCTAATTTTTGTTTTTTTGGTAGAGACGGGGTTTCACCATATTGGCCAGGCTGGTCTCCAACTCCTA |
| | ATCTCAGGTGATCTACCCACCTTGGCCTCCCAAATTGCTGGGATTACAGGCGTGAACCACTGCTCCCTTCCCTGTCCTTCTGATTTTAAAAAAAAGAGAAATGTTCTGGCACCTGCACTTGCACTGGGGACAGCCTATTTTGCTA |
| | GTTTGTTTTGTTTCGTTTTGTTTTGATGGAGAGCGTATGTTGTTTAAACGCGGCCGCGTAGATAAGTAGCATGGCGGGTTAATCATTAACTACAAGGAACCCCTAGTGATGGAGTTGGCCACTCCCTCTCTGCGCGCTCGCTCGC |
| | TCACTGAGGCCGGGCGACCAAAGGTCGCCCGACGCCCGGGCTTTGCCCGGGCGGCCTCAGTGAGCGAGCGAGCGCGCAG |
| 37 | >ITR-to-ITR construct with FVIII-QQ12 nucleic acid |
| | >[L-ITR]-[ACTUS101-LSP]-[5′ UTR]-[FVIII-QQ12(w/sp)]-[3′ UTR]-[PolIII_terminator]-[hGHpolyA]-[PolII_terminator-rev][R-ITR] |
| | CTGCGCGCTCGCTCGCTCACTGAGGCCGCCCGGGCAAAGCCCGGGCGTCGGGCGACCTTTGGTCGCCCGGCCTCAGTGAGCGAGCGAGCGCGCAGAGAGGGAGTGGCCAACTCCATCACTAGGGGTTCCTTGTAGTTAATGATTA |
| | ACCCGCCATGCTACTTATCTACGGCGCGCCACGCGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACT |
| | AGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACTAGTTCTAGAGCGGCCGCCAGTGTGCTGGAATTC |
| | GGCTTTTTTAGGGCTGGAAGCTACCTTTGACATCATTTCCTCTGCGAATGCATGTATAATTTCTACAGAACCTATTAGAAAGGATCACCCAGCCTCTGCTTTTGTACAACTTTCCCTTAAAAAACTGCCAATCCCACTGCTGTTT |
| | GGCCCAATAGTGAGAACTTTTTCCTGCTGCCTCTTGGTGCTTTTGCCTATGGCCCCTATTCTGCCTGCTGAAGACACTCTTGCCAGCATGGACTTAAACCCCTCCAGCTCTGACAATCCTCTTTCTCTTTTGTTTTACATGAAGG |
| | GTCTGGCAGCCAAAGCAATCACTCAAAGTTCAAACCTTATCATTTTTTGCTTTGTTCCTCTTGGCCTTGGTTTTGTACATCAGCTTTGAAAATACCATCCCAGGGTTAATGCTGGGGTTAATTTATAACTGAGAGTGCTCTAGTT |
| | CTGCAATACAGGACATGCTATAAAAATGGAAAGATGTTGCTTTCTGAGAGATCAGGTGGATTCTTGGGCATTTGCTTAAGCTTGGTACCGCCACCATGCAGATAGAACTGAGCACCTGCTTCTTCCTGTGCCTGCTGAGATTCTG |
| | CTTCTCTGCCACCAGAAGATACTACCTGGGGGCTGTGGAGCTGAGCTGGGACTACATGCAGTCTGACCTGGGAGAACTGCCTGTTGATGCTAGATTCCCCCCCAGAGTGCCCAAGAGCTTCCCCTTCAACACCTCAGTGGTGTAC |
| | AAGAAGACCCTGTTTGTAGAGTTCACTGACCACCTGTTCAACATTGCAAAGCCCAGACCCCCCTGGATGGGCCTGCTGGGCCCCACCATCCAGGCTGAGGTGTATGATACTGTTGTGATCACCCTGAAGAACATGGCCAGCCACC |
| | CTGTGAGCCTGCATGCTGTGGGAGTCAGCTACTGGAAGGCCTCAGAGGGGGCTGAGTATGATGACCAGACCAGCCAGAGAGAGAAGGAGGATGACAAGGTGTTCCCTGGTGGGAGCCACACCTATGTGTGGCAGGTGCTGAAGGA |
| | GAATGGACCCATGGCCTCAGACCCCCTGTGCCTGACCTACAGCTACCTGAGCCATGTAGACCTGGTGAAGGACCTGAACAGTGGTCTGATAGGTGCACTGCTGGTGTGCAGAGAGGGCAGCCTGGCCAAGGAGAAGACCCAGACC |
| | CTGCACAAGTTCATCCTGCTGTTTGCAGTGTTTGATGAGGGCAAGAGCTGGCACTCTGAGACCAAGAACAGCCTGATGCAGGACAGAGATGCTGCCTCAGCCAGAGCCTGGCCCAAGATGCACACTGTTAATGGGTATGTGAACA |
| | GAAGCCTGCCAGGCCTGATTGGCTGCCACAGAAAGAGTGTGTACTGGCATGTCATAGGAATGGGCACCACCCCTGAGGTGCACAGCATCTTCCTGGAGGGCCACACCTTCCTGGTGAGAAACCACAGACAGGCCAGCCTGGAGAT |
| | CAGCCCCATCACCTTCCTGACTGCACAGACCCTGCTGATGGACCTGGGCCAGTTCCTGCTGTTCTGCCACATCAGCAGCCACCAGCATGATGGGATGGAGGCCTATGTAAAGGTGGACAGCTGCCCTGAAGAGCCCCAGCTGCAG |
| | ATGAAGAACAATGAGGAGGCAGAAGACTATGATGATGATCTGACAGACAGTGAAATGGATGTGGTGAGATTTGATGATGACAACAGCCCCAGCTTCATCCAGATCAGAAGTGTGGCCAAGAAGCACCCCAAGACCTGGGTGCACT |
| | ACATTGCTGCAGAGGAGGAGGACTGGGACTATGCCCCCCTGGTGCTGGCCCCAGATGACAGAAGCTACAAGAGCCAGTACCTGAACAATGGGCCCCAGAGAATAGGGAGAAAGTACAAGAAGGTGAGATTCATGGCCTACACAGA |
| | TGAGACCTTCAAGACCAGAGAGGCCATCCAGCATGAGAGTGGTATCCTGGGCCCCCTGCTGTATGGAGAAGTGGGGGATACCCTGCTGATCATCTTCAAGAACCAGGCCAGCAGACCCTACAACATCTACCCCCATGGCATCACA |
| | GATGTGAGACCCCTGTACAGCAGAAGACTGCCCAAGGGTGTAAAGCACCTGAAGGACTTCCCCATCCTGCCAGGGGAAATCTTCAAGTACAAGTGGACTGTGACTGTGGAGGATGGACCCACCAAGAGTGATCCCAGATGCCTGA |
| | CCAGATACTACAGCAGCTTTGTTAACATGGAGAGAGACCTGGCCTCAGGACTGATTGGCCCCCTGCTGATCTGCTACAAGGAGAGTGTGGACCAGCAGGGCAACCAGATCATGAGTGACAAGAGAAATGTCATCCTGTTCAGTGT |
| | CTTTGATGAAAACAGAAGCTGGTACCTGACAGAAAACATCCAGAGATTCCTGCCCAACCCTGCAGGTGTGCAGCTGGAGGACCCAGAGTTCCAGGCCAGCAACATCATGCACAGCATCAATGGGTATGTGTTTGATAGCCTGCAG |
| | CTGAGTGTATGCCTGCATGAGGTGGCCTACTGGTACATCCTGAGCATAGGTGCTCAGACTGACTTCCTGTCAGTATTCTTCTCAGGCTACACCTTCAAGCACAAGATGGTGTATGAGGACACCCTGACCCTGTTCCCCTTCAGTG |
| | GGGAAACAGTGTTCATGAGCATGGAGAACCCTGGGCTGTGGATCCTGGGCTGCCACAACAGTGACTTCAGAAACAGAGGCATGACAGCCCTGCTGAAGGTGAGCAGCTGTGACAAGAACACAGGAGACTACTATGAAGACAGCTA |
| | TGAAGACATCAGTGCTTACCTGCTGAGCAAGAACAATGCAATAGAGCCCAGAAGCTTCAGCCAGAACCCCCCAGTGCTGAAGAGACACCAGAGAGAGATCACCAGAACCACCCTGCAGTCAGACCAGGAGGAGATTGACTATGAT |
| | GACACCATCTCTGTGGAGATGAAGAAGGAGGACTTTGACATCTATGATGAGGATGAGAACCAGAGCCCCAGAAGCTTCCAGAAGAAGACCAGACACTACTTCATAGCAGCAGTGGAGAGACTGTGGGACTATGGGATGAGCAGCA |
| | GCCCCCATGTCCTGAGAAACAGAGCCCAGTCAGGAAGTGTCCCCCAGTTCAAGAAGGTGGTGTTCCAGGAGTTCACTGATGGGAGCTTCACCCAGCCCCTGTACAGAGGAGAGCTGAATGAACACCTGGGCCTGCTGGGCCCCTA |
| | CATCAGAGCAGAGGTGGAGGACAACATCATGGTGACCTTCAGAAACCAGGCCAGCAGACCCTACAGCTTCTACAGCAGCCTGATCAGCTATGAGGAGGACCAGAGACAGGGAGCTGAACCCAGAAAGAACTTTGTGAAGCCCAAT |
| | GAAACCAAGACCTACTTCTGGAAGGTGCAGCACCACATGGCCCCCACCAAGGATGAGTTTGACTGCAAGGCCTGGGCCTACTTCTCTGATGTTGATCTGGAGAAGGATGTACACTCTGGGCTGATTGGCCCCCTGCTGGTGTGCC |
| | ACACCAACACCCTGAACCCAGCCCATGGCAGACAGGTGACTGTGCAGGAGTTTGCCCTGTTCTTCACCATCTTTGATGAGACCAAGAGCTGGTACTTCACTGAAAACATGGAGAGAAACTGCAGAGCCCCCTGCAACATCCAGAT |
| | GGAGGACCCCACCTTCAAGGAGAACTACAGATTCCATGCTATCAATGGCTACATCATGGACACCCTGCCTGGACTGGTGATGGCCCAGGACCAGAGAATCAGATGGTACCTGCTGAGCATGGGCAGCAATGAAAACATCCACAGC |
| | ATCCACTTCTCAGGCCATGTATTCACTGTGAGAAAGAAGGAGGAGTACAAGATGGCCCTGTACAACCTGTACCCAGGTGTGTTTGAAACAGTGGAAATGCTGCCCAGCAAGGCTGGCATCTGGAGAGTGGAGTGCCTGATTGGTG |
| | AGCACCTGCATGCTGGAATGAGCACCCTGTTCCTGGTGTACAGCAACAAGTGCCAGACCCCCCTGGGCATGGCCTCAGGGCACATCAGAGACTTCCAGATCACTGCCTCTGGCCAGTATGGACAGTGGGCCCCCAAGCTGGCCAG |
| | ACTGCACTACAGTGGCAGCATCAATGCCTGGAGCACCAAGGAGCCCTTCAGCTGGATCAAGGTGGACCTGCTGGCCCCCATGATCATCCATGGCATCAAGACCCAGGGTGCCAGACAGAAGTTCAGCAGCCTGTACATCAGCCAG |
| | TTCATCATCATGTACAGCCTGGATGGCAAGAAGTGGCAGACCTACAGAGGCAACAGCACAGGTACCCTGATGGTGTTCTTTGGCAATGTGGACAGCTCTGGTATCAAGCACAACATCTTCAACCCCCCCATCATAGCCAGATACA |
| | TCAGACTGCACCCCACCCACTACAGCATCAGAAGCACCCTGAGAATGGAGCTGATGGGCTGTGATCTGAACAGCTGCAGCATGCCCCTGGGCATGGAGAGCAAGGCCATCTCTGATGCTCAGATCACAGCTAGCAGCTACTTCAC |
| | CAACATGTTTGCCACCTGGAGCCCCAGCAAGGCCAGACTGCACCTGCAGGGCAGAAGCAATGCATGGAGACCCCAGGTGAACAACCCCAAGGAGTGGCTGCAGGTGGACTTCCAGAAGACCATGAAGGTGACAGGAGTGACCACC |
| | CAGGGAGTGAAGAGCCTGCTGACCAGCATGTATGTGAAGGAGTTCCTGATCAGCAGCAGCCAGGATGGCCACCAGTGGACCCTGTTCTTCCAGAATGGGAAGGTGAAGGTGTTCCAGGGCAACCAGGACAGCTTCACCCCAGTAG |
| | TGAACAGCCTGGACCCCCCCCTGCTGACCAGATACCTGAGAATCCACCCCCAGAGCTGGGTGCACCAGATAGCTCTGAGAATGGAGGTGCTGGGCTGTGAAGCCCAGGACCTGTACTGATTTAAATCTCGAGCCGGGCGGAGTGT |
| | GTTAGTCTCTCCAGAGGGAGGCTGGTTCCCCAGGGAAGCAGAGCCTGTGTGCGGGCAGCAGCTGTGTGCGGGCCTGGGGGTTGTTAAGTGCAATTATTTTTAATAAAAGGGGCATTTGGAAAAAAAAAAAAAAGGTAGCAGTCGA |
| | CAGATGAATTCTGCAGATCTGTGGCTTCTAGCTGCCCGGGTGGCATCCCTGTGACCCCTCCCCAGTGCCTCTCCTGGCCCTGGAAGTTGCCACTCCAGTGCCCACCAGCCTTGTCCTAATAAAATTAAGTTGCATCATTTTGTCT |
| | GACTAGGTGTCCTTCTATAATATTATGGGGTGGAGGGGGGTGGTATGGAGCAAGGGGCAAGTTGGGAAGACAACCTGTAGGGCCTGCGGGGTCTATTGGGAACCAAGCTGGAGTGCAGTGGCACAATCTTGGCTCACTGCAATCT |
| | CCGCCTCCTGGGTTCAAGCGATTCTCCTGCCTCAGCCTCCCGAGTTGTTGGGATTCCAGGCATGCATGACCAGGCTCAGCTAATTTTTGTTTTTTTGGTAGAGACGGGGTTTCACCATATTGGCCAGGCTGGTCTCCAACTCCTA |
| | ATCTCAGGTGATCTACCCACCTTGGCCTCCCAAATTGCTGGGATTACAGGCGTGAACCACTGCTCCCTTCCCTGTCCTTCTGATTTTAAAAAAAAGAGAAATGTTCTGGCACCTGCACTTGCACTGGGGACAGCCTATTTTGCTA |
| | GTTTGTTTTGTTTCGTTTTGTTTTGATGGAGAGCGTATGTTGTTTAAACGCGGCCGCGTAGATAAGTAGCATGGCGGGTTAATCATTAACTACAAGGAACCCCTAGTGATGGAGTTGGCCACTCCCTCTCTGCGCGCTCGCTCGC |
| | TCACTGAGGCCGGGCGACCAAAGGTCGCCCGACGCCCGGGCTTTGCCCGGGCGGCCTCAGTGAGCGAGCGAGCGCGCAG |
| 38 | >ITR-to-ITR construct with FVIII-QQ13 nucleic acid |
| | >[L-ITR]-[ACTUS101-LSP]-[5′ UTR]-[FVIII-QQ13(w/sp)]-[3′ UTR]-[PolIII_terminator]-[hGHpolyA]-[PolII_terminator-rev][R-ITR] |
| | CTGCGCGCTCGCTCGCTCACTGAGGCCGCCCGGGCAAAGCCCGGGCGTCGGGCGACCTTTGGTCGCCCGGCCTCAGTGAGCGAGCGAGCGCGCAGAGAGGGAGTGGCCAACTCCATCACTAGGGGTTCCTTGTAGTTAATGATTA |
| | ACCCGCCATGCTACTTATCTACGGCGCGCCACGCGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACT |
| | AGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACTAGTTCTAGAGCGGCCGCCAGTGTGCTGGAATTC |
| | GGCTTTTTTAGGGCTGGAAGCTACCTTTGACATCATTTCCTCTGCGAATGCATGTATAATTTCTACAGAACCTATTAGAAAGGATCACCCAGCCTCTGCTTTTGTACAACTTTCCCTTAAAAAACTGCCAATCCCACTGCTGTTT |
| | GGCCCAATAGTGAGAACTTTTTCCTGCTGCCTCTTGGTGCTTTTGCCTATGGCCCCTATTCTGCCTGCTGAAGACACTCTTGCCAGCATGGACTTAAACCCCTCCAGCTCTGACAATCCTCTTTCTCTTTTGTTTTACATGAAGG |
| | GTCTGGCAGCCAAAGCAATCACTCAAAGTTCAAACCTTATCATTTTTTGCTTTGTTCCTCTTGGCCTTGGTTTTGTACATCAGCTTTGAAAATACCATCCCAGGGTTAATGCTGGGGTTAATTTATAACTGAGAGTGCTCTAGTT |
| | CTGCAATACAGGACATGCTATAAAAATGGAAAGATGTTGCTTTCTGAGAGATCAGGTGGATTCTTGGGCATTTGCTTAAGCTTGGTACCGCCACCATGCAGATAGAACTGAGCACCTGCTTCTTCCTGTGCCTGCTGAGATTCTG |
| | CTTCAGTGCCACCAGAAGATACTACCTGGGTGCTGTTGAGCTGAGCTGGGACTACATGCAGTCTGACCTGGGTGAACTGCCAGTAGATGCAAGATTCCCCCCCAGAGTGCCCAAGAGCTTCCCCTTCAACACCTCTGTAGTCTAC |
| | AAGAAGACCCTGTTTGTGGAGTTCACAGATCACCTGTTCAACATTGCTAAGCCCAGACCCCCCTGGATGGGCCTGCTGGGCCCCACCATCCAGGCAGAGGTGTATGACACTGTAGTGATCACCCTGAAGAACATGGCCAGCCACC |
| | CAGTGAGCCTGCATGCTGTGGGTGTGAGCTACTGGAAGGCCAGTGAAGGAGCAGAGTATGATGATCAGACCAGCCAGAGAGAGAAGGAGGATGACAAGGTGTTCCCAGGTGGAAGCCACACCTATGTCTGGCAGGTGCTGAAGGA |
| | GAATGGGCCCATGGCCTCAGATCCCCTGTGCCTGACCTACAGCTACCTGAGCCATGTTGACCTGGTGAAGGACCTGAACAGTGGACTGATAGGTGCTCTGCTGGTGTGCAGAGAGGGCAGCCTGGCCAAGGAGAAGACCCAGACC |
| | CTGCACAAGTTCATCCTGCTGTTTGCAGTGTTTGATGAAGGCAAGAGCTGGCACAGTGAGACCAAGAACAGCCTGATGCAGGACAGAGATGCAGCATCAGCCAGAGCCTGGCCCAAGATGCACACAGTGAATGGCTATGTGAACA |
| | GAAGCCTGCCAGGCCTGATAGGATGCCACAGAAAGTCTGTATACTGGCATGTCATTGGTATGGGCACCACCCCTGAAGTGCACAGCATCTTCCTGGAGGGCCACACCTTCCTGGTGAGAAACCACAGACAGGCCAGCCTGGAGAT |
| | CAGCCCCATCACCTTCCTGACAGCCCAGACCCTGCTGATGGACCTGGGCCAGTTCCTGCTGTTCTGCCACATCAGCAGCCACCAGCATGATGGGATGGAGGCCTATGTCAAGGTGGACAGCTGCCCAGAGGAGCCCCAGCTGCAG |
| | ATGAAGAACAATGAGGAGGCTGAGGACTATGATGATGACCTGACTGACTCAGAGATGGATGTGGTGAGATTTGATGATGATAACAGCCCCAGCTTCATCCAGATCAGATCTGTGGCCAAGAAGCACCCCAAGACCTGGGTGCACT |
| | ACATAGCTGCAGAAGAGGAGGACTGGGACTATGCCCCCCTGGTGCTGGCCCCAGATGATAGAAGCTACAAGAGCCAGTACCTGAACAATGGCCCCCAGAGAATTGGGAGAAAGTACAAGAAGGTGAGATTCATGGCCTACACAGA |
| | TGAAACCTTCAAGACCAGAGAGGCCATCCAGCATGAGTCTGGCATCCTGGGCCCCCTGCTGTATGGGGAAGTGGGAGACACCCTGCTGATCATCTTCAAGAACCAGGCCAGCAGACCCTACAACATCTACCCCCATGGTATCACA |
| | GATGTGAGACCCCTGTACAGCAGAAGACTGCCCAAGGGGGTGAAGCACCTGAAGGACTTCCCCATCCTGCCTGGGGAGATCTTCAAGTACAAGTGGACAGTAACTGTGGAGGATGGCCCCACCAAGTCTGACCCCAGATGCCTGA |
| | CCAGATACTACAGCAGCTTTGTGAACATGGAGAGAGACCTGGCCAGTGGCCTGATTGGCCCCCTGCTGATCTGCTACAAGGAGAGTGTTGACCAGCAGGGCAACCAGATCATGAGTGATAAGAGAAATGTTATCCTGTTCAGTGT |
| | GTTTGATGAAAACAGAAGCTGGTACCTGACAGAGAACATCCAGAGATTCCTGCCCAACCCAGCTGGTGTCCAGCTGGAGGACCCTGAGTTCCAGGCCAGCAACATCATGCACAGCATCAATGGCTATGTATTTGACAGCCTGCAG |
| | CTGAGTGTGTGCCTGCATGAAGTGGCCTACTGGTACATCCTGAGCATAGGTGCTCAGACAGACTTCCTGAGTGTCTTCTTCAGTGGCTACACCTTCAAGCACAAGATGGTGTATGAAGACACCCTGACCCTGTTCCCCTTCAGTG |
| | GAGAGACAGTGTTCATGAGCATGGAGAACCCAGGACTGTGGATCCTGGGCTGCCACAACAGTGACTTCAGAAACAGAGGCATGACAGCCCTGCTGAAGGTGAGCAGCTGTGACAAGAACACTGGGGACTACTATGAGGACAGCTA |
| | TGAGGACATCTCTGCCTACCTGCTGAGCAAGAACAATGCAATAGAGCCCAGAAGCTTCAGCCAGAACCCCCCTGTGCTGAAGAGACACCAGAGAGAGATCACCAGAACCACCCTGCAGTCAGATCAGGAGGAGATAGACTATGAT |
| | GATACCATCTCTGTGGAGATGAAGAAGGAGGACTTTGATATCTATGATGAGGATGAGAACCAGAGCCCCAGAAGCTTCCAGAAGAAGACCAGACACTACTTCATAGCTGCAGTGGAGAGACTGTGGGACTATGGCATGAGCAGCA |
| | GCCCCCATGTACTGAGAAACAGAGCCCAGTCAGGATCTGTCCCCCAGTTCAAGAAGGTGGTGTTCCAGGAGTTCACTGATGGGAGCTTCACCCAGCCCCTGTACAGAGGTGAACTGAATGAACACCTGGGCCTGCTGGGCCCCTA |
| | CATCAGAGCAGAGGTGGAGGACAACATCATGGTGACCTTCAGAAACCAGGCCAGCAGACCCTACAGCTTCTACAGCAGCCTGATCAGCTATGAAGAGGACCAGAGACAGGGAGCTGAGCCCAGAAAGAACTTTGTGAAGCCCAAT |
| | GAAACCAAGACCTACTTCTGGAAGGTGCAGCACCACATGGCCCCCACCAAGGATGAATTTGACTGCAAGGCCTGGGCCTACTTCTCAGATGTAGACCTGGAGAAGGATGTGCACTCAGGACTGATTGGCCCCCTGCTGGTGTGCC |
| | ACACCAACACCCTGAACCCAGCACATGGCAGACAGGTGACAGTTCAGGAGTTTGCACTGTTCTTCACCATCTTTGATGAGACCAAGAGCTGGTACTTCACAGAGAACATGGAGAGAAACTGCAGAGCCCCCTGCAACATCCAGAT |
| | GGAGGACCCCACCTTCAAGGAGAACTACAGATTCCATGCTATCAATGGCTACATCATGGACACCCTGCCAGGGCTGGTGATGGCCCAGGACCAGAGAATCAGATGGTACCTGCTGAGCATGGGCAGCAATGAAAACATCCACAGC |
| | ATCCACTTCAGTGGCCATGTGTTCACAGTTAGAAAGAAGGAGGAGTACAAGATGGCCCTGTACAACCTGTACCCTGGTGTGTTTGAAACAGTGGAGATGCTGCCCAGCAAGGCTGGCATCTGGAGAGTGGAGTGCCTGATTGGGG |
| | AACACCTGCATGCAGGAATGAGCACCCTGTTCCTGGTGTACAGCAACAAGTGCCAGACCCCCCTGGGCATGGCCTCAGGCCACATCAGAGACTTCCAGATCACTGCCAGTGGGCAGTATGGTCAGTGGGCCCCCAAGCTGGCCAG |
| | ACTGCACTACTCTGGCAGCATCAATGCCTGGAGCACCAAGGAGCCCTTCAGCTGGATCAAGGTGGACCTGCTGGCCCCCATGATCATCCATGGCATCAAGACCCAGGGGGCCAGACAGAAGTTCAGCAGCCTGTACATCAGCCAG |
| | TTCATCATCATGTACAGCCTGGATGGCAAGAAGTGGCAGACCTACAGAGGCAACAGCACTGGAACCCTGATGGTGTTCTTTGGCAATGTAGACAGCTCAGGCATCAAGCACAACATCTTCAACCCCCCCATCATTGCCAGATACA |
| | TCAGACTGCACCCCACCCACTACAGCATCAGAAGCACCCTGAGAATGGAGCTGATGGGCTGTGACCTGAACAGCTGCAGCATGCCCCTGGGCATGGAGAGCAAGGCCATCTCTGATGCCCAGATCACTGCCAGCAGCTACTTCAC |
| | CAACATGTTTGCCACCTGGAGCCCCAGCAAGGCCAGACTGCACCTGCAGGGCAGAAGCAATGCTTGGAGACCCCAGGTGAACAACCCCAAGGAGTGGCTGCAGGTGGACTTCCAGAAGACCATGAAGGTGACTGGTGTGACCACC |
| | CAGGGTGTGAAGAGCCTGCTGACCAGCATGTATGTCAAGGAGTTCCTGATCAGCAGCAGCCAGGATGGTCACCAGTGGACCCTGTTCTTCCAGAATGGCAAGGTGAAGGTGTTCCAGGGCAACCAGGACAGCTTCACCCCAGTGG |
| | TGAACAGCCTGGACCCCCCCCTGCTGACCAGATACCTGAGAATCCACCCCCAGAGCTGGGTGCACCAGATAGCCCTGAGAATGGAGGTGCTGGGCTGTGAGGCCCAGGACCTGTACTGATTTAAATCTCGAGCCGGGCGGAGTGT |
| | GTTAGTCTCTCCAGAGGGAGGCTGGTTCCCCAGGGAAGCAGAGCCTGTGTGCGGGCAGCAGCTGTGTGCGGGCCTGGGGGTTGTTAAGTGCAATTATTTTTAATAAAAGGGGCATTTGGAAAAAAAAAAAAAAGGTAGCAGTCGA |
| | CAGATGAATTCTGCAGATCTGTGGCTTCTAGCTGCCCGGGTGGCATCCCTGTGACCCCTCCCCAGTGCCTCTCCTGGCCCTGGAAGTTGCCACTCCAGTGCCCACCAGCCTTGTCCTAATAAAATTAAGTTGCATCATTTTGTCT |
| | GACTAGGTGTCCTTCTATAATATTATGGGGTGGAGGGGGGTGGTATGGAGCAAGGGGCAAGTTGGGAAGACAACCTGTAGGGCCTGCGGGGTCTATTGGGAACCAAGCTGGAGTGCAGTGGCACAATCTTGGCTCACTGCAATCT |
| | CCGCCTCCTGGGTTCAAGCGATTCTCCTGCCTCAGCCTCCCGAGTTGTTGGGATTCCAGGCATGCATGACCAGGCTCAGCTAATTTTTGTTTTTTTGGTAGAGACGGGGTTTCACCATATTGGCCAGGCTGGTCTCCAACTCCTA |
| | ATCTCAGGTGATCTACCCACCTTGGCCTCCCAAATTGCTGGGATTACAGGCGTGAACCACTGCTCCCTTCCCTGTCCTTCTGATTTTAAAAAAAAGAGAAATGTTCTGGCACCTGCACTTGCACTGGGGACAGCCTATTTTGCTA |
| | GTTTGTTTTGTTTCGTTTTGTTTTGATGGAGAGCGTATGTTGTTTAAACGCGGCCGCGTAGATAAGTAGCATGGCGGGTTAATCATTAACTACAAGGAACCCCTAGTGATGGAGTTGGCCACTCCCTCTCTGCGCGCTCGCTCGC |
| | TCACTGAGGCCGGGCGACCAAAGGTCGCCCGACGCCCGGGCTTTGCCCGGGCGGCCTCAGTGAGCGAGCGAGCGCGCAG |
| 39 | >ITR-to-ITR construct with FVIII-QQ14 nucleic acid |
| | >[L-ITR]-[ACTUS101-LSP]-[5′ UTR]-[FVIII-QQ14(w/sp)]-[3′ UTR]-[PolIII_terminator]-[hGHpolyA]-[PolII_terminator-rev][R-ITR] |
| | CTGCGCGCTCGCTCGCTCACTGAGGCCGCCCGGGCAAAGCCCGGGCGTCGGGCGACCTTTGGTCGCCCGGCCTCAGTGAGCGAGCGAGCGCGCAGAGAGGGAGTGGCCAACTCCATCACTAGGGGTTCCTTGTAGTTAATGATTA |
| | ACCCGCCATGCTACTTATCTACGGCGCGCCACGCGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACT |
| | AGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACTAGTTCTAGAGCGGCCGCCAGTGTGCTGGAATTC |
| | GGCTTTTTTAGGGCTGGAAGCTACCTTTGACATCATTTCCTCTGCGAATGCATGTATAATTTCTACAGAACCTATTAGAAAGGATCACCCAGCCTCTGCTTTTGTACAACTTTCCCTTAAAAAACTGCCAATCCCACTGCTGTTT |
| | GGCCCAATAGTGAGAACTTTTTCCTGCTGCCTCTTGGTGCTTTTGCCTATGGCCCCTATTCTGCCTGCTGAAGACACTCTTGCCAGCATGGACTTAAACCCCTCCAGCTCTGACAATCCTCTTTCTCTTTTGTTTTACATGAAGG |
| | GTCTGGCAGCCAAAGCAATCACTCAAAGTTCAAACCTTATCATTTTTTGCTTTGTTCCTCTTGGCCTTGGTTTTGTACATCAGCTTTGAAAATACCATCCCAGGGTTAATGCTGGGGTTAATTTATAACTGAGAGTGCTCTAGTT |
| | CTGCAATACAGGACATGCTATAAAAATGGAAAGATGTTGCTTTCTGAGAGATCAGGTGGATTCTTGGGCATTTGCTTAAGCTTGGTACCGCCACCATGCAGATTGAACTGAGCACCTGCTTCTTCCTGTGCCTGCTGAGATTCTG |
| | CTTCAGTGCCACCAGAAGATACTACCTGGGGGCAGTGGAGCTGAGCTGGGACTACATGCAGAGTGACCTGGGGGAACTGCCTGTTGATGCCAGATTCCCCCCCAGAGTGCCCAAGAGCTTCCCCTTCAACACCTCAGTTGTGTAC |
| | AAGAAGACCCTGTTTGTGGAGTTCACTGACCACCTGTTCAACATTGCTAAGCCCAGACCCCCCTGGATGGGCCTGCTGGGCCCCACCATCCAGGCTGAGGTGTATGACACTGTGGTGATCACCCTGAAGAACATGGCCAGCCACC |
| | CTGTGAGCCTGCATGCAGTGGGGGTGAGCTACTGGAAGGCCTCAGAAGGGGCAGAGTATGATGATCAGACCAGCCAGAGAGAGAAGGAGGATGACAAGGTGTTCCCTGGTGGTAGCCACACCTATGTGTGGCAGGTGCTGAAGGA |
| | GAATGGCCCCATGGCCTCAGACCCCCTGTGCCTGACCTACAGCTACCTGAGCCATGTGGACCTGGTGAAGGACCTGAACAGTGGCCTGATTGGAGCTCTGCTGGTGTGCAGAGAGGGCAGCCTGGCCAAGGAGAAGACCCAGACC |
| | CTGCACAAGTTCATCCTGCTGTTTGCTGTGTTTGATGAGGGCAAGAGCTGGCACTCAGAGACCAAGAACAGCCTGATGCAGGACAGAGATGCTGCCAGTGCAAGAGCCTGGCCCAAGATGCACACTGTCAATGGATATGTGAACA |
| | GAAGCCTGCCAGGACTGATAGGCTGCCACAGAAAGTCTGTATACTGGCATGTTATAGGTATGGGCACCACCCCAGAGGTGCACAGCATCTTCCTGGAGGGCCACACCTTCCTGGTGAGAAACCACAGACAGGCCAGCCTGGAGAT |
| | CAGCCCCATCACCTTCCTGACTGCCCAGACCCTGCTGATGGACCTGGGCCAGTTCCTGCTGTTCTGCCACATCAGCAGCCACCAGCATGATGGTATGGAGGCCTATGTGAAGGTGGACAGCTGCCCTGAGGAGCCCCAGCTGCAG |
| | ATGAAGAACAATGAGGAGGCAGAGGACTATGATGATGACCTGACAGACAGTGAGATGGATGTGGTGAGATTTGATGATGACAACAGCCCCAGCTTCATCCAGATCAGAAGTGTGGCCAAGAAGCACCCCAAGACCTGGGTGCACT |
| | ACATTGCTGCTGAAGAGGAGGACTGGGACTATGCACCCCTGGTGCTGGCCCCAGATGATAGAAGCTACAAGAGCCAGTACCTGAACAATGGACCCCAGAGAATTGGAAGAAAGTACAAGAAGGTGAGATTCATGGCCTACACTGA |
| | TGAGACCTTCAAGACCAGAGAGGCCATCCAGCATGAATCAGGGATCCTGGGCCCCCTGCTGTATGGAGAGGTGGGAGACACCCTGCTGATCATCTTCAAGAACCAGGCCAGCAGACCCTACAACATCTACCCCCATGGTATCACT |
| | GATGTAAGACCCCTGTACAGCAGAAGACTGCCCAAGGGAGTGAAGCACCTGAAGGACTTCCCCATCCTGCCTGGGGAAATCTTCAAGTACAAGTGGACAGTCACAGTTGAAGATGGACCCACCAAGAGTGATCCCAGATGCCTGA |
| | CCAGATACTACAGCAGCTTTGTGAACATGGAGAGAGACCTGGCCTCAGGCCTGATAGGCCCCCTGCTGATCTGCTACAAGGAGTCTGTTGACCAGCAGGGCAACCAGATCATGAGTGACAAGAGAAATGTGATCCTGTTCTCAGT |
| | GTTTGATGAGAACAGAAGCTGGTACCTGACTGAAAACATCCAGAGATTCCTGCCCAACCCAGCAGGGGTGCAGCTGGAGGACCCAGAGTTCCAGGCCAGCAACATCATGCACAGCATCAATGGCTATGTCTTTGATAGCCTGCAG |
| | CTGTCTGTATGCCTGCATGAGGTGGCCTACTGGTACATCCTGAGCATAGGAGCCCAGACTGATTTCCTGAGTGTTTTCTTCAGTGGCTACACCTTCAAGCACAAGATGGTGTATGAGGACACCCTGACCCTGTTCCCCTTCTCTG |
| | GTGAGACTGTGTTCATGAGCATGGAGAACCCTGGTCTGTGGATCCTGGGCTGCCACAACTCAGATTTCAGAAACAGAGGCATGACAGCCCTGCTGAAGGTGAGCAGCTGTGACAAGAACACTGGGGACTACTATGAAGACAGCTA |
| | TGAAGACATCTCTGCTTACCTGCTGAGCAAGAACAATGCCATTGAGCCCAGAAGCTTCAGCCAGAACCCCCCTGTGCTGAAGAGACACCAGAGAGAGATCACCAGAACCACCCTGCAGTCAGACCAGGAGGAGATAGACTATGAT |
| | GATACCATCTCTGTGGAGATGAAGAAGGAGGACTTTGATATCTATGATGAGGATGAAAACCAGAGCCCCAGAAGCTTCCAGAAGAAGACCAGACACTACTTCATAGCAGCTGTAGAGAGACTGTGGGACTATGGCATGAGCAGCA |
| | GCCCCCATGTACTGAGAAACAGAGCCCAGAGTGGAAGTGTGCCCCAGTTCAAGAAGGTGGTGTTCCAGGAGTTCACAGATGGCAGCTTCACCCAGCCCCTGTACAGAGGAGAACTGAATGAGCACCTGGGCCTGCTGGGCCCCTA |
| | CATCAGAGCTGAGGTGGAGGACAACATCATGGTGACCTTCAGAAACCAGGCCAGCAGACCCTACAGCTTCTACAGCAGCCTGATCAGCTATGAGGAGGACCAGAGACAGGGAGCTGAGCCCAGAAAGAACTTTGTCAAGCCCAAT |
| | GAGACCAAGACCTACTTCTGGAAGGTGCAGCACCACATGGCCCCCACCAAGGATGAATTTGACTGCAAGGCCTGGGCCTACTTCTCAGATGTGGACCTGGAGAAGGATGTGCACTCAGGTCTGATTGGCCCCCTGCTGGTGTGCC |
| | ACACCAACACCCTGAACCCAGCCCATGGTAGACAGGTGACTGTGCAGGAGTTTGCTCTGTTCTTCACCATCTTTGATGAGACCAAGAGCTGGTACTTCACAGAGAACATGGAGAGAAACTGCAGAGCCCCCTGCAACATCCAGAT |
| | GGAGGACCCCACCTTCAAGGAGAACTACAGATTCCATGCCATCAATGGGTACATCATGGACACCCTGCCAGGTCTGGTGATGGCCCAGGACCAGAGAATCAGATGGTACCTGCTGAGCATGGGCAGCAATGAAAACATCCACAGC |
| | ATCCACTTCAGTGGACATGTGTTCACAGTTAGAAAGAAGGAGGAGTACAAGATGGCCCTGTACAACCTGTACCCTGGAGTGTTTGAAACTGTGGAGATGCTGCCCAGCAAGGCAGGCATCTGGAGAGTGGAGTGCCTGATAGGGG |
| | AGCACCTGCATGCTGGTATGAGCACCCTGTTCCTGGTGTACAGCAACAAGTGCCAGACCCCCCTGGGCATGGCCTCAGGCCACATCAGAGACTTCCAGATCACTGCTAGTGGCCAGTATGGTCAGTGGGCCCCCAAGCTGGCCAG |
| | ACTGCACTACTCTGGAAGCATCAATGCCTGGAGCACCAAGGAGCCCTTCAGCTGGATCAAGGTGGACCTGCTGGCCCCCATGATCATCCATGGGATCAAGACCCAGGGGGCCAGACAGAAGTTCAGCAGCCTGTACATCAGCCAG |
| | TTCATCATCATGTACAGCCTGGATGGAAAGAAGTGGCAGACCTACAGAGGCAACAGCACAGGCACCCTGATGGTGTTCTTTGGCAATGTAGACAGCTCAGGCATCAAGCACAACATCTTCAACCCCCCCATCATAGCAAGATACA |
| | TCAGACTGCACCCCACCCACTACAGCATCAGAAGCACCCTGAGAATGGAGCTGATGGGCTGTGACCTGAACAGCTGCAGCATGCCCCTGGGCATGGAGAGCAAGGCCATCTCAGATGCCCAGATCACAGCCAGCAGCTACTTCAC |
| | CAACATGTTTGCCACCTGGAGCCCCAGCAAGGCCAGACTGCACCTGCAGGGCAGAAGCAATGCCTGGAGACCCCAGGTGAACAACCCCAAGGAGTGGCTGCAGGTGGACTTCCAGAAGACCATGAAGGTGACTGGTGTGACCACC |
| | CAGGGTGTTAAGAGCCTGCTGACCAGCATGTATGTGAAGGAGTTCCTGATCAGCAGCAGCCAGGATGGGCACCAGTGGACCCTGTTCTTCCAGAATGGAAAGGTGAAGGTGTTCCAGGGCAACCAGGACAGCTTCACCCCTGTTG |
| | TGAACAGCCTGGACCCCCCCCTGCTGACCAGATACCTGAGAATCCACCCCCAGAGCTGGGTGCACCAGATAGCACTGAGAATGGAGGTGCTGGGCTGTGAGGCCCAGGACCTGTACTGATTTAAATCTCGAGCCGGGCGGAGTGT |
| | GTTAGTCTCTCCAGAGGGAGGCTGGTTCCCCAGGGAAGCAGAGCCTGTGTGCGGGCAGCAGCTGTGTGCGGGCCTGGGGGTTGTTAAGTGCAATTATTTTTAATAAAAGGGGCATTTGGAAAAAAAAAAAAAAGGTAGCAGTCGA |
| | CAGATGAATTCTGCAGATCTGTGGCTTCTAGCTGCCCGGGTGGCATCCCTGTGACCCCTCCCCAGTGCCTCTCCTGGCCCTGGAAGTTGCCACTCCAGTGCCCACCAGCCTTGTCCTAATAAAATTAAGTTGCATCATTTTGTCT |
| | GACTAGGTGTCCTTCTATAATATTATGGGGTGGAGGGGGGTGGTATGGAGCAAGGGGCAAGTTGGGAAGACAACCTGTAGGGCCTGCGGGGTCTATTGGGAACCAAGCTGGAGTGCAGTGGCACAATCTTGGCTCACTGCAATCT |
| | CCGCCTCCTGGGTTCAAGCGATTCTCCTGCCTCAGCCTCCCGAGTTGTTGGGATTCCAGGCATGCATGACCAGGCTCAGCTAATTTTTGTTTTTTTGGTAGAGACGGGGTTTCACCATATTGGCCAGGCTGGTCTCCAACTCCTA |
| | ATCTCAGGTGATCTACCCACCTTGGCCTCCCAAATTGCTGGGATTACAGGCGTGAACCACTGCTCCCTTCCCTGTCCTTCTGATTTTAAAAAAAAGAGAAATGTTCTGGCACCTGCACTTGCACTGGGGACAGCCTATTTTGCTA |
| | GTTTGTTTTGTTTCGTTTTGTTTTGATGGAGAGCGTATGTTGTTTAAACGCGGCCGCGTAGATAAGTAGCATGGCGGGTTAATCATTAACTACAAGGAACCCCTAGTGATGGAGTTGGCCACTCCCTCTCTGCGCGCTCGCTCGC |
| | TCACTGAGGCCGGGCGACCAAAGGTCGCCCGACGCCCGGGCTTTGCCCGGGCGGCCTCAGTGAGCGAGCGAGCGCGCAG |
| 40 | >5′ UTR\|SERPINA7 |
| | CAGGTGGATTCTTGGGCATTTGCTTAAGCTTGGTACCGCCACC |
| 41 | >5′ UTR\|24BP |
| | CAGGTGGATTCTTGGGCATTTGCT |
| 42 | >FL-POLYA |
| | ATCTGTGGCTTCTAGCTGCCCGGGTGGCATCCCTGTGACCCCTCCCCAGTGCCTCTCCTGGCCCTGGAAGTTGCCACTCCAGTGCCCACCAGCCTTGTCCTAATAAAATTAAGTTGCATCATTTTGTCTGACTAGGTGTCCTTCT |
| | ATAATATTATGGGGTGGAGGGGGGTGGTATGGAGCAAGGGGCAAGTTGGG |
| 43 | CTGTGGCTTCTAGCTGCCCGGGTGGCATCCCTGTGACCCCTCCCCAGTGCCTCTCCTGGCCCTGGAAGTTGCCACTCCAGTGCCCACCAGCCTTGTCCTAATAAAATTAAGTTGCATCATTTTGTCTGACTAGGTGTCCTTCTAT |
| | AATATTATGGGGTGGAGGGGGGTGGTATGGAGCAAGGGGCAAGTTGGG |
| 44 | >FL-POLYA plus AATAA signal |
| | CTGTGGCTTCTAGCTGCCCGGGTGGCATCCCTGTGACCCCTCCCCAGTGCCTCTCCTGGCCCTGGAAGTTGCCACTCCAGTGCCCACCAGCCTTGTCCTAATAAAATTAAGTTGCATCATTTTGTCTGACTAGGTGTCCTTCTAT |
| | AATATTATGGGGTGGAGGGGGGTGGTATGGAGCAAGGGGCAAGTTGGG |
| 45 | >REV RNA POLII_TERMINATOR |
| | AGAGAAATGTTCTGGCACCTGCACTTGCACTGGGGACAGCCTATTTTGCTAGTTTGTTTTGTTTCGTTTTGTTTTGATGGAGAGCGTATGTT |
| 46 | >Stuffer DNA with 48 bp synthetic polyA |
| | GTCCTTCAACGTCGAACTGTAGCACTCTGGATAGCTAGCTAGCGTTTGTAGATAGTTTCAAGGATAGCAACCAACAACACGATAGTTTAGAAATTGTTCTAGATCTTCTGCTACTACTCGAGAGATAGTTGACTTCTAGAAGACT |
| | CTGGATAGAGAGCTTCCTGTGGTTTCTATTTCAACACTTCGAGCGATTTAGATAGAGGCGTCAATTGAGAACAAAAACTGGACACCTGTTTCTAGAACACTAGTGCTAGTCTTCTCAAGGCTTGATACGTCCTGCCACTCACACA |
| | CAAAAAACCAACACACAGATTAATGAAAATAAAGATCTTTTATTAGAGAGCGAGTGGATAGTTTAGAGTCGCTTGAGACTTATCCTGTGTCCAACTAGCCAAGAGTAGAGAGAGAGTGACTAGGTGGATCGAGAGATCGTCGTCT |
| | ATTTGACTGCTAGCCTGTGTTCTCGTCGTAGTCCAAATCTTCAATACTCTGGCGACTAGTAACTTGAATCTGTGTATCTAACACGACAAGGCTACTCCTTTGAGTTTCTAGTGATAAGTGCGACTGGATCTAGCTAGTTGAGTTT |
| | CTAGAAACTACGTCCAAAGCTACTAGCACTA |
| 47 | >synthetic polyA |
| | CACACAAAAAACCAACACACAGATTAATGAAAATAAAGATCTTTTATT |
| 48 | >3′ UTR |
| | AGCCGGGCGGAGTGTGTTAGTCTCTCCAGAGGGAGGCTGGTTCCCCAGGGAAGCAGAGCCTGTGTGCGGGCAGCAGCTGTGTGCGGGCCTGGGGGTTG |
| 49 | >3′ UTR |
| | CCGGGCGGAGTGTGTTAGTCTCTCCAGAGGGAGGCTGGTTCCCCAGGGAAGCAGAGCCTGTGTGCGGGCAGCAGCTGTGTGCGGGCCTGGGGGTTG |
| 50 | >ITR-to-ITR construct with FVIII-QQ15 nucleic acid |
| | >[L-ITR]-[ACTUS101-LSP]-[5′ UTR]-[FVIII-QQ15(w/sp)]-[3′ UTR]-[PolIII_terminator]-[hGHpolyA]-[PolII_terminator-rev][R-ITR] |
| | CTGCGCGCTCGCTCGCTCACTGAGGCCGCCCGGGCAAAGCCCGGGCGTCGGGCGACCTTTGGTCGCCCGGCCTCAGTGAGCGAGCGAGCGCGCAGAGAGGGAGTGGCCAACTCCATCACTAGGGGTTCCTTGTAGTTAATGATTA |
| | ACCCGCCATGCTACTTATCTACGGCGCGCCACGCGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACT |
| | AGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACTAGTTCTAGAGCGGCCGCCAGTGTGCTGGAATTC |
| | GGCTTTTTTAGGGCTGGAAGCTACCTTTGACATCATTTCCTCTGCGAATGCATGTATAATTTCTACAGAACCTATTAGAAAGGATCACCCAGCCTCTGCTTTTGTACAACTTTCCCTTAAAAAACTGCCAATCCCACTGCTGTTT |
| | GGCCCAATAGTGAGAACTTTTTCCTGCTGCCTCTTGGTGCTTTTGCCTATGGCCCCTATTCTGCCTGCTGAAGACACTCTTGCCAGCATGGACTTAAACCCCTCCAGCTCTGACAATCCTCTTTCTCTTTTGTTTTACATGAAGG |
| | GTCTGGCAGCCAAAGCAATCACTCAAAGTTCAAACCTTATCATTTTTTGCTTTGTTCCTCTTGGCCTTGGTTTTGTACATCAGCTTTGAAAATACCATCCCAGGGTTAATGCTGGGGTTAATTTATAACTGAGAGTGCTCTAGTT |
| | CTGCAATACAGGACATGCTATAAAAATGGAAAGATGTTGCTTTCTGAGAGATCAGGTGGATTCTTGGGCATTTGCTTAAGCTTGGTACCGCCACCATGCAGATTGAACTGAGCACCTGCTTCTTCCTGTGCCTGCTGAGATTCTG |
| | CTTCAGTGCTACCAGAAGATACTACCTGGGGGCAGTGGAGCTGAGCTGGGACTACATGCAGTCTGACCTGGGAGAGCTGCCAGTAGATGCCAGATTCCCCCCCAGAGTGCCCAAGAGCTTCCCCTTCAACACCTCTGTGGTGTAC |
| | AAGAAGACCCTGTTTGTGGAGTTCACAGACCACCTGTTCAACATAGCTAAGCCCAGACCCCCCTGGATGGGCCTGCTGGGCCCCACCATCCAGGCAGAGGTGTATGACACTGTTGTGATCACCCTGAAGAACATGGCCAGCCACC |
| | CAGTTAGCCTGCATGCTGTTGGTGTTAGCTACTGGAAGGCCTCTGAGGGAGCAGAGTATGATGACCAGACCAGCCAGAGAGAGAAGGAGGATGACAAGGTGTTCCCAGGGGGGAGCCACACCTATGTCTGGCAGGTGCTGAAGGA |
| | GAATGGCCCCATGGCCTCAGACCCCCTGTGCCTGACCTACAGCTACCTGAGCCATGTGGACCTGGTGAAGGACCTGAACTCTGGCCTGATTGGTGCACTGCTGGTGTGCAGAGAGGGCAGCCTGGCCAAGGAGAAGACCCAGACC |
| | CTGCACAAGTTCATCCTGCTGTTTGCTGTCTTTGATGAGGGCAAGAGCTGGCACTCAGAAACCAAGAACAGCCTGATGCAGGACAGAGATGCAGCTTCTGCTAGAGCCTGGCCCAAGATGCACACTGTGAATGGCTATGTCAACA |
| | GAAGCCTGCCTGGCCTGATAGGCTGCCACAGAAAGTCTGTGTACTGGCATGTGATTGGGATGGGCACCACCCCAGAGGTGCACAGCATCTTCCTGGAGGGCCACACCTTCCTGGTGAGAAACCACAGACAGGCCAGCCTGGAGAT |
| | CAGCCCCATCACCTTCCTGACTGCCCAGACCCTGCTGATGGACCTGGGCCAGTTCCTGCTGTTCTGCCACATCAGCAGCCACCAGCATGATGGTATGGAGGCCTATGTGAAGGTGGACAGCTGCCCAGAGGAGCCCCAGCTGCAG |
| | ATGAAGAACAATGAAGAGGCAGAGGACTATGATGATGACCTGACAGACTCAGAAATGGATGTGGTGAGATTTGATGATGATAACAGCCCCAGCTTCATCCAGATCAGATCTGTTGCCAAGAAGCACCCCAAGACCTGGGTGCACT |
| | ACATTGCAGCTGAGGAGGAGGACTGGGACTATGCTCCCCTGGTGCTGGCCCCAGATGACAGAAGCTACAAGAGCCAGTACCTGAACAATGGACCCCAGAGAATAGGGAGAAAGTACAAGAAGGTGAGATTCATGGCCTACACAGA |
| | TGAGACCTTCAAGACCAGAGAGGCCATCCAGCATGAATCAGGGATCCTGGGCCCCCTGCTGTATGGGGAGGTGGGTGACACCCTGCTGATCATCTTCAAGAACCAGGCCAGCAGACCCTACAACATCTACCCCCATGGAATCACA |
| | GATGTCAGACCCCTGTACAGCAGAAGACTGCCCAAGGGAGTAAAGCACCTGAAGGACTTCCCCATCCTGCCAGGTGAGATCTTCAAGTACAAGTGGACAGTAACAGTGGAGGATGGTCCCACCAAGTCTGACCCCAGATGCCTGA |
| | CCAGATACTACAGCAGCTTTGTTAACATGGAGAGAGACCTGGCCAGTGGCCTGATAGGCCCCCTGCTGATCTGCTACAAGGAGTCTGTAGACCAGCAGGGCAACCAGATCATGTCAGATAAGAGAAATGTAATCCTGTTCAGTGT |
| | GTTTGATGAGAACAGAAGCTGGTACCTGACAGAGAACATCCAGAGATTCCTGCCCAACCCTGCAGGTGTGCAGCTGGAGGACCCTGAATTCCAGGCCAGCAACATCATGCACAGCATCAATGGATATGTTTTTGACAGCCTGCAG |
| | CTGTCAGTTTGCCTGCATGAGGTGGCCTACTGGTACATCCTGAGCATTGGGGCCCAGACAGATTTCCTGTCTGTGTTCTTCAGTGGGTACACCTTCAAGCACAAGATGGTGTATGAGGACACCCTGACCCTGTTCCCCTTCTCAG |
| | GTGAAACTGTGTTCATGAGCATGGAGAACCCAGGGCTGTGGATCCTGGGCTGCCACAACTCAGACTTCAGAAACAGAGGCATGACTGCTCTGCTGAAGGTGAGCAGCTGTGACAAGAACACAGGTGACTACTATGAGGACAGCTA |
| | TGAAGACATCTCAGCTTACCTGCTGAGCAAGAACAATGCCATAGAGCCCAGAAGCTTCAGCCAGAACCCCCCAGTTCTGAAGAGACACCAGAGAGAGATCACCAGAACCACCCTGCAGAGTGACCAGGAGGAGATTGACTATGAT |
| | GACACCATCTCTGTAGAGATGAAGAAGGAGGACTTTGACATCTATGATGAGGATGAGAACCAGAGCCCCAGAAGCTTCCAGAAGAAGACCAGACACTACTTCATAGCTGCAGTGGAGAGACTGTGGGACTATGGCATGAGCAGCA |
| | GCCCCCATGTGCTGAGAAACAGAGCCCAGAGTGGCTCAGTGCCCCAGTTCAAGAAGGTGGTGTTCCAGGAGTTCACAGATGGCAGCTTCACCCAGCCCCTGTACAGAGGGGAGCTGAATGAGCACCTGGGCCTGCTGGGCCCCTA |
| | CATCAGAGCTGAGGTGGAGGACAACATCATGGTGACCTTCAGAAACCAGGCCAGCAGACCCTACAGCTTCTACAGCAGCCTGATCAGCTATGAGGAGGACCAGAGACAGGGGGCAGAGCCCAGAAAGAACTTTGTAAAGCCCAAT |
| | GAAACCAAGACCTACTTCTGGAAGGTGCAGCACCACATGGCCCCCACCAAGGATGAATTTGACTGCAAGGCCTGGGCCTACTTCAGTGATGTGGACCTGGAGAAGGATGTGCACTCAGGCCTGATTGGTCCCCTGCTGGTGTGCC |
| | ACACCAACACCCTGAACCCAGCCCATGGCAGACAGGTGACTGTACAGGAGTTTGCCCTGTTCTTCACCATCTTTGATGAGACCAAGAGCTGGTACTTCACAGAGAACATGGAGAGAAACTGCAGAGCCCCCTGCAACATCCAGAT |
| | GGAGGACCCCACCTTCAAGGAGAACTACAGATTCCATGCTATCAATGGGTACATCATGGACACCCTGCCAGGCCTGGTGATGGCCCAGGACCAGAGAATCAGATGGTACCTGCTGAGCATGGGCAGCAATGAGAACATCCACAGC |
| | ATCCACTTCAGTGGCCATGTGTTCACAGTGAGAAAGAAGGAGGAGTACAAGATGGCCCTGTACAACCTGTACCCTGGAGTGTTTGAAACTGTGGAGATGCTGCCCAGCAAGGCAGGTATCTGGAGAGTGGAGTGCCTGATTGGTG |
| | AGCACCTGCATGCTGGGATGAGCACCCTGTTCCTGGTGTACAGCAACAAGTGCCAGACCCCCCTGGGCATGGCCTCAGGGCACATCAGAGACTTCCAGATCACTGCCAGTGGGCAGTATGGGCAGTGGGCCCCCAAGCTGGCCAG |
| | ACTGCACTACAGTGGAAGCATCAATGCATGGAGCACCAAGGAGCCCTTCAGCTGGATCAAGGTGGACCTGCTGGCCCCCATGATCATCCATGGGATCAAGACCCAGGGGGCCAGACAGAAGTTCAGCAGCCTGTACATCAGCCAG |
| | TTCATCATCATGTACAGCCTGGATGGCAAGAAGTGGCAGACCTACAGAGGCAACAGCACAGGTACCCTGATGGTGTTCTTTGGAAATGTGGACAGCTCAGGTATCAAGCACAACATCTTCAACCCCCCCATCATAGCAAGATACA |
| | TCAGACTGCACCCCACCCACTACAGCATCAGAAGCACCCTGAGAATGGAGCTGATGGGCTGTGACCTGAACAGCTGCAGCATGCCCCTGGGCATGGAGAGCAAGGCCATCTCAGATGCTCAGATCACTGCCAGCAGCTACTTCAC |
| | CAACATGTTTGCCACCTGGAGCCCCAGCAAGGCCAGACTGCACCTGCAGGGCAGAAGCAATGCCTGGAGACCCCAGGTGAACAACCCCAAGGAGTGGCTGCAGGTGGACTTCCAGAAGACCATGAAGGTGACAGGGGTTACCACC |
| | CAGGGAGTGAAGAGCCTGCTGACCAGCATGTATGTAAAGGAGTTCCTGATCAGCAGCAGCCAGGATGGTCACCAGTGGACCCTGTTCTTCCAGAATGGAAAGGTGAAGGTGTTCCAGGGCAACCAGGACAGCTTCACCCCAGTGG |
| | TGAACAGCCTGGACCCCCCCCTGCTGACCAGATACCTGAGAATCCACCCCCAGAGCTGGGTGCACCAGATAGCCCTGAGAATGGAGGTGCTGGGCTGTGAAGCCCAGGACCTGTACTGATTTAAATCTCGAGCCGGGCGGAGTGT |
| | GTTAGTCTCTCCAGAGGGAGGCTGGTTCCCCAGGGAAGCAGAGCCTGTGTGCGGGCAGCAGCTGTGTGCGGGCCTGGGGGTTGTTAAGTGCAATTATTTTTAATAAAAGGGGCATTTGGAAAAAAAAAAAAAAGGTAGCAGTCGA |
| | CAGATGAATTCTGCAGATCTGTGGCTTCTAGCTGCCCGGGTGGCATCCCTGTGACCCCTCCCCAGTGCCTCTCCTGGCCCTGGAAGTTGCCACTCCAGTGCCCACCAGCCTTGTCCTAATAAAATTAAGTTGCATCATTTTGTCT |
| | GACTAGGTGTCCTTCTATAATATTATGGGGTGGAGGGGGGTGGTATGGAGCAAGGGGCAAGTTGGGAAGACAACCTGTAGGGCCTGCGGGGTCTATTGGGAACCAAGCTGGAGTGCAGTGGCACAATCTTGGCTCACTGCAATCT |
| | CCGCCTCCTGGGTTCAAGCGATTCTCCTGCCTCAGCCTCCCGAGTTGTTGGGATTCCAGGCATGCATGACCAGGCTCAGCTAATTTTTGTTTTTTTGGTAGAGACGGGGTTTCACCATATTGGCCAGGCTGGTCTCCAACTCCTA |
| | ATCTCAGGTGATCTACCCACCTTGGCCTCCCAAATTGCTGGGATTACAGGCGTGAACCACTGCTCCCTTCCCTGTCCTTCTGATTTTAAAAAAAAGAGAAATGTTCTGGCACCTGCACTTGCACTGGGGACAGCCTATTTTGCTA |
| | GTTTGTTTTGTTTCGTTTTGTTTTGATGGAGAGCGTATGTTGTTTAAACGCGGCCGCGTAGATAAGTAGCATGGCGGGTTAATCATTAACTACAAGGAACCCCTAGTGATGGAGTTGGCCACTCCCTCTCTGCGCGCTCGCTCGC |
| | TCACTGAGGCCGGGCGACCAAAGGTCGCCCGACGCCCGGGCTTTGCCCGGGCGGCCTCAGTGAGCGAGCGAGCGCGCAG |
| 51 | >ITR-to-ITR construct with FVIII-QQ16 nucleic acid |
| | >[L-ITR]-[ACTUS101 -LSP]-[5′ UTR]-[FVIII-QQ16(w/sp)]-[3′ UTR]-[PolIII_terminator]-[hGHpolyA]-[PolII_terminator-rev][R-ITR] |
| | CTGCGCGCTCGCTCGCTCACTGAGGCCGCCCGGGCAAAGCCCGGGCGTCGGGCGACCTTTGGTCGCCCGGCCTCAGTGAGCGAGCGAGCGCGCAGAGAGGGAGTGGCCAACTCCATCACTAGGGGTTCCTTGTAGTTAATGATTA |
| | ACCCGCCATGCTACTTATCTACGGCGCGCCACGCGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACT |
| | AGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACTAGTTCTAGAGCGGCCGCCAGTGTGCTGGAATTC |
| | GGCTTTTTTAGGGCTGGAAGCTACCTTTGACATCATTTCCTCTGCGAATGCATGTATAATTTCTACAGAACCTATTAGAAAGGATCACCCAGCCTCTGCTTTTGTACAACTTTCCCTTAAAAAACTGCCAATCCCACTGCTGTTT |
| | GGCCCAATAGTGAGAACTTTTTCCTGCTGCCTCTTGGTGCTTTTGCCTATGGCCCCTATTCTGCCTGCTGAAGACACTCTTGCCAGCATGGACTTAAACCCCTCCAGCTCTGACAATCCTCTTTCTCTTTTGTTTTACATGAAGG |
| | GTCTGGCAGCCAAAGCAATCACTCAAAGTTCAAACCTTATCATTTTTTGCTTTGTTCCTCTTGGCCTTGGTTTTGTACATCAGCTTTGAAAATACCATCCCAGGGTTAATGCTGGGGTTAATTTATAACTGAGAGTGCTCTAGTT |
| | CTGCAATACAGGACATGCTATAAAAATGGAAAGATGTTGCTTTCTGAGAGATCAGGTGGATTCTTGGGCATTTGCTTAAGCTTGGTACCGCCACCATGCAAATAGAACTCTCCACCTGCTTCTTTCTGTGCCTTTTGAGGTTCTG |
| | CTTTTCTGCAACTAGAAGATACTACCTGGGTGCAGTAGAACTGTCATGGGACTATATGCAATCAGATCTAGGAGAACTGCCAGTGGATGCCAGATTTCCTCCAAGGGTGCCCAAATCATTTCCATTCAACACAAGTGTTGTGTAC |
| | AAAAAGACCCTGTTTGTGGAATTCACAGATCACCTTTTCAACATAGCCAAGCCTAGACCACCTTGGATGGGTCTGTTGGGTCCTACCATCCAGGCAGAAGTTTATGATACTGTGGTCATTACACTTAAGAACATGGCTAGCCATC |
| | CAGTCAGTCTTCATGCTGTTGGGGTATCCTACTGGAAAGCTTCAGAGGGAGCAGAATATGATGACCAGACAAGTCAAAGGGAGAAAGAAGATGACAAAGTCTTCCCTGGTGGAAGCCATACCTATGTCTGGCAGGTCCTGAAAGA |
| | GAATGGGCCAATGGCCAGTGACCCACTGTGCCTTACCTACTCTTATCTTAGTCATGTGGACCTGGTAAAAGACTTGAATAGTGGCCTCATTGGAGCACTACTGGTATGTAGAGAAGGGAGCTTGGCTAAGGAAAAGACACAGACT |
| | TTGCACAAATTTATACTACTTTTTGCTGTATTTGATGAGGGGAAAAGTTGGCACAGTGAAACAAAGAACTCCTTGATGCAGGATAGGGATGCAGCAAGTGCTAGGGCTTGGCCAAAAATGCACACAGTTAATGGGTATGTTAACA |
| | GATCACTGCCTGGGCTGATTGGTTGCCACAGAAAATCTGTCTATTGGCATGTGATTGGAATGGGCACAACTCCTGAAGTGCACTCAATATTCCTTGAAGGTCACACATTTCTTGTGAGAAACCATAGACAGGCTTCCTTGGAAAT |
| | CAGCCCCATAACCTTCCTTACAGCTCAAACACTCTTGATGGACCTTGGACAGTTTCTACTGTTTTGTCATATCTCTTCCCACCAACATGATGGAATGGAAGCCTATGTCAAAGTAGACAGCTGTCCTGAGGAACCACAACTACAG |
| | ATGAAAAATAATGAAGAAGCAGAAGACTATGATGATGATCTTACTGATTCAGAAATGGATGTGGTCAGGTTTGATGATGATAACTCTCCTTCCTTTATCCAAATTAGATCTGTAGCAAAGAAGCATCCTAAAACTTGGGTACATT |
| | ACATTGCTGCAGAAGAGGAGGACTGGGACTATGCACCCTTGGTCTTGGCACCAGATGATAGAAGTTATAAAAGTCAATATTTGAACAATGGCCCCCAGAGAATTGGTAGGAAGTACAAAAAAGTCAGATTTATGGCATACACAGA |
| | TGAAACATTTAAGACAAGAGAAGCTATTCAGCATGAGTCAGGGATCTTGGGACCTTTACTTTATGGTGAAGTTGGAGATACACTGTTGATTATATTTAAGAATCAAGCAAGCAGACCATATAACATCTACCCTCATGGTATCACA |
| | GATGTAAGACCCTTGTATTCAAGAAGATTACCCAAAGGTGTAAAACATTTGAAGGATTTTCCAATTCTGCCAGGGGAGATATTCAAATATAAATGGACAGTGACAGTAGAGGATGGGCCAACTAAATCAGATCCTAGGTGCCTGA |
| | CCAGGTATTACTCTAGTTTTGTTAATATGGAGAGAGATCTAGCTAGTGGACTCATTGGACCTCTCCTCATCTGCTACAAAGAATCAGTTGATCAACAGGGAAACCAGATAATGTCAGATAAGAGGAATGTCATCCTGTTTTCTGT |
| | ATTTGATGAAAACAGGAGCTGGTACCTCACAGAAAATATACAAAGATTTCTCCCCAATCCTGCTGGAGTGCAGCTTGAAGATCCAGAATTCCAAGCTTCAAACATCATGCACAGCATCAATGGCTATGTATTTGATTCCTTGCAG |
| | TTGTCAGTGTGTTTGCATGAGGTGGCATACTGGTACATTCTATCTATTGGAGCTCAGACTGACTTCCTTTCTGTGTTCTTCTCAGGATATACATTCAAACACAAAATGGTCTATGAAGACACACTCACCCTATTCCCATTCTCAG |
| | GGGAAACAGTCTTCATGTCAATGGAAAACCCAGGTCTATGGATTTTGGGGTGCCACAACTCAGACTTTAGAAACAGAGGCATGACAGCTTTACTGAAGGTTAGTAGTTGTGATAAGAACACTGGGGATTATTATGAGGACTCTTA |
| | TGAAGATATTTCTGCATACTTGCTGAGTAAAAACAATGCAATTGAACCAAGAAGCTTCTCTCAAAACCCCCCAGTCTTGAAAAGACATCAAAGAGAAATAACCAGAACTACTCTTCAGTCTGATCAAGAGGAAATTGACTATGAT |
| | GATACCATATCAGTTGAAATGAAGAAGGAAGATTTTGACATTTATGATGAAGATGAAAATCAGAGCCCCAGGAGCTTTCAAAAGAAAACAAGACACTATTTTATTGCAGCAGTGGAGAGGCTCTGGGATTATGGTATGAGTAGCT |
| | CCCCACATGTACTAAGGAACAGGGCCCAGAGTGGCTCTGTCCCACAGTTCAAGAAAGTAGTTTTCCAGGAATTTACAGATGGCTCCTTTACTCAGCCTTTATACAGGGGAGAACTAAATGAACATTTGGGACTCCTAGGGCCATA |
| | TATAAGAGCAGAGGTTGAAGATAATATCATGGTAACTTTCAGGAATCAGGCCTCTAGGCCCTATTCCTTCTATTCTAGCCTTATTTCTTATGAGGAAGATCAGAGGCAAGGAGCAGAGCCTAGGAAAAACTTTGTTAAGCCTAAT |
| | GAAACCAAAACATACTTTTGGAAAGTGCAACATCATATGGCCCCCACCAAAGATGAATTTGACTGCAAAGCCTGGGCATATTTCTCTGATGTGGACTTAGAAAAAGATGTTCACTCAGGCCTGATAGGACCACTTCTGGTCTGCC |
| | ACACTAACACACTGAACCCAGCCCATGGGAGACAAGTGACTGTACAGGAATTTGCACTGTTTTTCACCATCTTTGATGAAACAAAAAGCTGGTACTTCACTGAAAATATGGAAAGAAACTGCAGGGCTCCCTGCAATATCCAGAT |
| | GGAAGATCCAACATTTAAAGAGAATTATAGATTCCATGCTATCAATGGCTACATAATGGATACACTACCAGGCTTAGTAATGGCTCAGGATCAAAGGATTAGATGGTATCTGCTCAGCATGGGCAGTAATGAGAACATCCATTCT |
| | ATTCATTTCTCAGGACATGTATTCACTGTAAGAAAAAAAGAGGAGTATAAAATGGCACTGTACAATCTCTATCCAGGTGTTTTTGAGACAGTAGAAATGTTACCTTCAAAAGCAGGAATTTGGAGAGTGGAATGCCTTATTGGTG |
| | AGCATCTACATGCTGGAATGAGCACACTTTTTCTGGTGTACAGCAATAAGTGTCAGACCCCTCTGGGAATGGCAAGTGGACACATTAGAGACTTTCAGATTACAGCTTCAGGACAATATGGACAGTGGGCCCCAAAGCTGGCCAG |
| | ACTTCATTATTCAGGGTCAATCAATGCATGGAGCACCAAGGAGCCTTTTAGCTGGATCAAGGTGGATCTGTTGGCACCAATGATTATTCATGGCATCAAGACTCAGGGAGCCAGACAGAAGTTCTCAAGCCTCTACATCTCTCAG |
| | TTTATCATCATGTATTCCCTTGATGGGAAGAAGTGGCAGACTTATAGAGGAAATAGTACAGGAACCTTAATGGTCTTCTTTGGGAATGTGGATTCAAGTGGGATAAAACACAATATTTTTAACCCTCCAATTATTGCTAGGTACA |
| | TCAGGTTGCACCCCACTCATTATAGCATTAGGAGCACTCTTAGAATGGAGTTGATGGGCTGTGATTTAAATAGTTGCAGTATGCCATTGGGAATGGAGAGTAAAGCAATATCAGATGCACAGATTACTGCATCATCTTACTTTAC |
| | TAATATGTTTGCAACTTGGTCTCCATCAAAAGCAAGACTTCACCTCCAAGGGAGGTCTAATGCTTGGAGACCTCAGGTGAATAATCCAAAAGAGTGGCTGCAAGTGGACTTCCAGAAGACAATGAAAGTCACAGGTGTGACCACT |
| | CAGGGTGTAAAATCTCTGCTTACCAGCATGTATGTTAAGGAGTTCCTCATCTCCAGCAGTCAAGATGGACATCAGTGGACTCTCTTTTTTCAGAATGGTAAAGTAAAGGTTTTTCAGGGAAATCAAGACTCCTTCACACCAGTGG |
| | TTAACAGTCTAGACCCACCATTACTGACTAGGTACCTTAGAATTCACCCACAGAGTTGGGTGCACCAGATTGCCCTGAGGATGGAGGTTCTGGGCTGTGAAGCACAGGACCTCTACTGATTTAAATCTCGAGCCGGGCGGAGTGT |
| | GTTAGTCTCTCCAGAGGGAGGCTGGTTCCCCAGGGAAGCAGAGCCTGTGTGCGGGCAGCAGCTGTGTGCGGGCCTGGGGGTTGTTAAGTGCAATTATTTTTAATAAAAGGGGCATTTGGAAAAAAAAAAAAAAGGTAGCAGTCGA |
| | CAGATGAATTCTGCAGATCTGTGGCTTCTAGCTGCCCGGGTGGCATCCCTGTGACCCCTCCCCAGTGCCTCTCCTGGCCCTGGAAGTTGCCACTCCAGTGCCCACCAGCCTTGTCCTAATAAAATTAAGTTGCATCATTTTGTCT |
| | GACTAGGTGTCCTTCTATAATATTATGGGGTGGAGGGGGGTGGTATGGAGCAAGGGGCAAGTTGGGAAGACAACCTGTAGGGCCTGCGGGGTCTATTGGGAACCAAGCTGGAGTGCAGTGGCACAATCTTGGCTCACTGCAATCT |
| | CCGCCTCCTGGGTTCAAGCGATTCTCCTGCCTCAGCCTCCCGAGTTGTTGGGATTCCAGGCATGCATGACCAGGCTCAGCTAATTTTTGTTTTTTTGGTAGAGACGGGGTTTCACCATATTGGCCAGGCTGGTCTCCAACTCCTA |
| | ATCTCAGGTGATCTACCCACCTTGGCCTCCCAAATTGCTGGGATTACAGGCGTGAACCACTGCTCCCTTCCCTGTCCTTCTGATTTTAAAAAAAAGAGAAATGTTCTGGCACCTGCACTTGCACTGGGGACAGCCTATTTTGCTA |
| | GTTTGTTTTGTTTCGTTTTGTTTTGATGGAGAGCGTATGTTGTTTAAACGCGGCCGCGTAGATAAGTAGCATGGCGGGTTAATCATTAACTACAAGGAACCCCTAGTGATGGAGTTGGCCACTCCCTCTCTGCGCGCTCGCTCGC |
| | TCACTGAGGCCGGGCGACCAAAGGTCGCCCGACGCCCGGGCTTTGCCCGGGCGGCCTCAGTGAGCGAGCGAGCGCGCAG |
| 52 | >ITR-to-ITR construct with FVIII-QQ17 nucleic acid |
| | >[L-ITR]-[ACTUS101-LSP]-[5′ UTR]-[FVIII-QQ17(w/sp)]-[3′ UTR]-[PolIII_terminator]-[hGHpolyA]-[PolII_terminator-rev][R-ITR] |
| | CTGCGCGCTCGCTCGCTCACTGAGGCCGCCCGGGCAAAGCCCGGGCGTCGGGCGACCTTTGGTCGCCCGGCCTCAGTGAGCGAGCGAGCGCGCAGAGAGGGAGTGGCCAACTCCATCACTAGGGGTTCCTTGTAGTTAATGATTA |
| | ACCCGCCATGCTACTTATCTACGGCGCGCCACGCGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACT |
| | AGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACTAGTTCTAGAGCGGCCGCCAGTGTGCTGGAATTC |
| | GGCTTTTTTAGGGCTGGAAGCTACCTTTGACATCATTTCCTCTGCGAATGCATGTATAATTTCTACAGAACCTATTAGAAAGGATCACCCAGCCTCTGCTTTTGTACAACTTTCCCTTAAAAAACTGCCAATCCCACTGCTGTTT |
| | GGCCCAATAGTGAGAACTTTTTCCTGCTGCCTCTTGGTGCTTTTGCCTATGGCCCCTATTCTGCCTGCTGAAGACACTCTTGCCAGCATGGACTTAAACCCCTCCAGCTCTGACAATCCTCTTTCTCTTTTGTTTTACATGAAGG |
| | GTCTGGCAGCCAAAGCAATCACTCAAAGTTCAAACCTTATCATTTTTTGCTTTGTTCCTCTTGGCCTTGGTTTTGTACATCAGCTTTGAAAATACCATCCCAGGGTTAATGCTGGGGTTAATTTATAACTGAGAGTGCTCTAGTT |
| | CTGCAATACAGGACATGCTATAAAAATGGAAAGATGTTGCTTTCTGAGAGATCAGGTGGATTCTTGGGCATTTGCTTAAGCTTGGTACCGCCACCATGCAAATAGAGCTCTCCACCTGCTTCTTTCTGTGCCTTTTGAGGTTCTG |
| | CTTTTCTGCCACAAGGAGATACTACCTGGGTGCAGTGGAACTGTCATGGGACTATATGCAAAGTGACCTAGGAGAGCTGCCAGTGGATGCAAGGTTTCCTCCTAGAGTGCCCAAAAGTTTTCCATTCAACACATCAGTTGTGTAC |
| | AAAAAGACACTGTTTGTAGAATTCACAGATCACCTTTTCAACATAGCAAAGCCCAGGCCACCATGGATGGGTCTGCTAGGTCCTACCATCCAGGCAGAGGTTTATGATACTGTGGTCATTACACTTAAGAACATGGCTTCACATC |
| | CTGTCAGTCTTCATGCTGTGGGTGTATCCTACTGGAAAGCTTCAGAGGGAGCAGAATATGATGACCAGACTAGTCAAAGAGAGAAAGAAGATGATAAAGTCTTCCCAGGTGGATCCCATACCTATGTCTGGCAGGTCCTGAAAGA |
| | GAATGGTCCTATGGCCAGTGATCCTCTGTGCCTTACTTACAGTTATCTTAGTCATGTTGACCTGGTAAAAGACTTGAATTCAGGACTCATTGGAGCACTATTGGTATGTAGAGAAGGGTCTTTAGCTAAGGAAAAGACACAGACC |
| | TTGCACAAATTTATACTACTTTTTGCAGTATTTGATGAAGGGAAAAGTTGGCACAGTGAAACAAAGAACTCCTTGATGCAGGATAGGGATGCAGCATCTGCTAGGGCCTGGCCCAAAATGCACACAGTCAATGGGTATGTGAACA |
| | GGTCTCTGCCTGGTCTGATTGGATGCCACAGAAAATCAGTATATTGGCATGTGATAGGAATGGGCACAACTCCTGAGGTGCACTCAATATTCTTAGAAGGTCACACATTTCTTGTAAGAAACCATAGACAGGCATCCTTGGAAAT |
| | CTCTCCCATAACATTCCTTACAGCTCAAACCCTCTTGATGGACCTTGGACAGTTTCTACTGTTTTGTCATATCTCTTCCCACCAACATGATGGGATGGAAGCATATGTCAAAGTAGACAGCTGTCCAGAGGAACCACAACTACAG |
| | ATGAAAAATAATGAAGAAGCAGAAGACTATGATGATGATCTTACTGATTCAGAAATGGATGTAGTCAGGTTTGATGATGATAACTCTCCTTCCTTTATCCAAATTAGGTCTGTTGCCAAGAAGCATCCTAAAACTTGGGTACATT |
| | ACATTGCTGCAGAAGAGGAGGACTGGGACTATGCACCATTAGTACTAGCACCTGATGACAGAAGTTATAAAAGTCAATATTTGAACAATGGACCCCAGAGAATTGGGAGGAAGTACAAAAAAGTCAGATTTATGGCATACACTGA |
| | TGAGACATTTAAGACTAGAGAAGCTATTCAGCATGAATCAGGAATCTTGGGACCTTTACTTTATGGGGAAGTAGGAGACACACTGTTGATTATATTTAAGAATCAAGCAAGCAGACCATATAACATCTACCCACATGGAATCACA |
| | GATGTTAGACCCTTGTATTCAAGGAGGTTACCAAAAGGTGTAAAACATTTGAAGGATTTTCCTATTCTGCCAGGTGAAATATTCAAATATAAATGGACTGTGACTGTTGAAGATGGACCAACTAAATCAGACCCCAGGTGCCTGA |
| | CCAGGTATTACTCTAGTTTTGTTAATATGGAGAGAGATCTAGCTTCAGGACTCATAGGCCCTCTCCTCATCTGCTACAAAGAATCAGTAGATCAACAGGGAAACCAGATAATGTCAGACAAGAGGAATGTAATCCTGTTTTCTGT |
| | ATTTGATGAAAACAGAAGCTGGTACCTCACAGAGAATATACAAAGGTTTCTCCCCAATCCTGCAGGAGTGCAGCTTGAGGATCCTGAGTTCCAAGCTTCCAACATCATGCACAGCATCAATGGGTATGTGTTTGATTCTTTGCAG |
| | TTGTCTGTTTGTTTGCATGAAGTGGCATACTGGTACATTCTATCTATTGGTGCTCAGACTGACTTCCTTTCTGTATTCTTCTCAGGCTATACTTTCAAACACAAAATGGTCTATGAAGACACCCTCACCCTATTCCCATTCTCTG |
| | GGGAAACTGTGTTCATGTCCATGGAAAACCCTGGACTATGGATTCTGGGCTGCCACAACTCTGACTTTAGAAACAGAGGCATGACTGCTTTACTGAAGGTTTCAAGTTGTGACAAGAACACTGGTGATTATTATGAAGACTCCTA |
| | TGAAGATATTAGTGCCTACTTGCTGAGTAAAAACAATGCAATAGAACCAAGGAGCTTCTCTCAAAACCCCCCAGTCTTGAAAAGGCATCAAAGAGAAATAACCAGAACTACTCTTCAGTCAGATCAAGAGGAAATTGACTATGAT |
| | GATACCATAAGTGTTGAAATGAAGAAGGAAGATTTTGACATTTATGATGAGGATGAAAATCAGAGCCCCAGGAGCTTTCAAAAGAAAACAAGACACTATTTTATAGCTGCTGTAGAGAGACTCTGGGATTATGGGATGAGTTCAT |
| | CCCCTCATGTACTAAGGAACAGGGCCCAGTCAGGTTCAGTCCCCCAGTTCAAGAAAGTTGTCTTCCAGGAATTTACAGATGGGTCCTTTACTCAGCCTTTATACAGGGGAGAACTAAATGAACATTTGGGACTCCTGGGACCATA |
| | TATAAGAGCAGAAGTTGAAGATAATATCATGGTAACTTTCAGGAATCAGGCCTCTAGACCCTATTCCTTCTATTCTAGCCTTATTTCTTATGAAGAAGATCAGAGGCAAGGAGCAGAACCTAGAAAAAACTTTGTCAAGCCTAAT |
| | GAAACTAAAACTTACTTTTGGAAAGTGCAACATCATATGGCTCCCACTAAAGATGAGTTTGATTGCAAAGCCTGGGCATATTTCTCAGATGTTGACCTAGAGAAAGATGTACACTCAGGACTGATTGGACCTCTTCTGGTCTGCC |
| | ACACTAACACACTGAACCCTGCTCATGGTAGACAAGTGACTGTACAGGAATTTGCCCTGTTTTTCACCATCTTTGATGAGACAAAAAGCTGGTACTTCACAGAAAATATGGAAAGAAACTGCAGGGCTCCCTGCAATATCCAGAT |
| | GGAAGATCCTACCTTTAAAGAGAATTATAGGTTCCATGCAATCAATGGCTACATAATGGATACACTACCTGGGTTAGTAATGGCTCAGGATCAAAGGATTAGGTGGTATCTGCTCAGCATGGGCTCCAATGAGAACATCCATTCC |
| | ATTCATTTCTCAGGACATGTGTTCACAGTTAGGAAAAAAGAGGAGTATAAAATGGCACTGTACAATCTCTATCCAGGTGTTTTTGAAACAGTAGAAATGTTACCCAGTAAAGCTGGAATTTGGAGGGTGGAGTGCCTTATAGGTG |
| | AGCATCTACATGCAGGAATGAGCACACTTTTTCTGGTGTACAGCAATAAGTGTCAGACACCCCTGGGAATGGCCTCAGGACACATTAGGGACTTTCAGATTACAGCCAGTGGACAATATGGTCAGTGGGCCCCAAAGCTGGCCAG |
| | ACTTCATTATAGTGGTTCAATCAATGCTTGGAGCACTAAGGAGCCCTTTTCTTGGATCAAGGTGGATCTGCTGGCTCCCATGATTATTCATGGCATCAAGACACAGGGTGCTAGGCAGAAGTTCAGCAGTCTCTACATCTCTCAG |
| | TTTATCATCATGTATTCACTTGATGGGAAGAAGTGGCAGACTTATAGAGGAAATTCCACAGGAACCTTAATGGTCTTCTTTGGAAATGTGGATTCATCTGGAATAAAACACAATATTTTTAACCCTCCAATTATTGCTAGATACA |
| | TCAGGTTGCACCCAACTCATTATAGCATTAGAAGCACTCTTAGAATGGAGTTGATGGGCTGTGATTTAAATAGTTGCTCCATGCCCTTGGGAATGGAGAGTAAAGCAATATCAGATGCCCAGATTACAGCTTCATCATACTTTAC |
| | CAATATGTTTGCAACATGGTCTCCATCAAAAGCAAGACTTCACCTCCAAGGGAGAAGTAATGCCTGGAGACCTCAGGTGAATAATCCAAAAGAGTGGCTGCAAGTGGACTTCCAGAAGACTATGAAAGTCACAGGAGTAACAACT |
| | CAGGGAGTTAAATCTCTGCTTACCAGCATGTATGTAAAGGAGTTCCTCATCTCCAGCAGTCAAGATGGGCATCAGTGGACTCTCTTTTTTCAGAATGGAAAAGTAAAGGTTTTTCAGGGAAATCAAGACTCCTTCACACCAGTGG |
| | TTAACAGTCTGGACCCTCCCTTACTGACTAGATACCTTAGGATTCACCCTCAGAGTTGGGTGCACCAGATTGCACTGAGAATGGAGGTTCTGGGCTGTGAGGCACAGGACCTCTACTGATTTAAATCTCGAGCCGGGCGGAGTGT |
| | GTTAGTCTCTCCAGAGGGAGGCTGGTTCCCCAGGGAAGCAGAGCCTGTGTGCGGGCAGCAGCTGTGTGCGGGCCTGGGGGTTGTTAAGTGCAATTATTTTTAATAAAAGGGGCATTTGGAAAAAAAAAAAAAAGGTAGCAGTCGA |
| | CAGATGAATTCTGCAGATCTGTGGCTTCTAGCTGCCCGGGTGGCATCCCTGTGACCCCTCCCCAGTGCCTCTCCTGGCCCTGGAAGTTGCCACTCCAGTGCCCACCAGCCTTGTCCTAATAAAATTAAGTTGCATCATTTTGTCT |
| | GACTAGGTGTCCTTCTATAATATTATGGGGTGGAGGGGGGTGGTATGGAGCAAGGGGCAAGTTGGGAAGACAACCTGTAGGGCCTGCGGGGTCTATTGGGAACCAAGCTGGAGTGCAGTGGCACAATCTTGGCTCACTGCAATCT |
| | CCGCCTCCTGGGTTCAAGCGATTCTCCTGCCTCAGCCTCCCGAGTTGTTGGGATTCCAGGCATGCATGACCAGGCTCAGCTAATTTTTGTTTTTTTGGTAGAGACGGGGTTTCACCATATTGGCCAGGCTGGTCTCCAACTCCTA |
| | ATCTCAGGTGATCTACCCACCTTGGCCTCCCAAATTGCTGGGATTACAGGCGTGAACCACTGCTCCCTTCCCTGTCCTTCTGATTTTAAAAAAAAGAGAAATGTTCTGGCACCTGCACTTGCACTGGGGACAGCCTATTTTGCTA |
| | GTTTGTTTTGTTTCGTTTTGTTTTGATGGAGAGCGTATGTTGTTTAAACGCGGCCGCGTAGATAAGTAGCATGGCGGGTTAATCATTAACTACAAGGAACCCCTAGTGATGGAGTTGGCCACTCCCTCTCTGCGCGCTCGCTCGC |
| | TCACTGAGGCCGGGCGACCAAAGGTCGCCCGACGCCCGGGCTTTGCCCGGGCGGCCTCAGTGAGCGAGCGAGCGCGCAG |
| 53 | >ITR-to-ITR construct with FVIII-QQ18 nucleic acid |
| | >[L-ITR]-[ACTUS101-LSP]-[5′ UTR]-[ FVIII-QQ18(w/sp)]-[3′ UTR]-[PolIII_terminator]-[hGHpolyA]-[PolII_terminator-rev][R-ITR] |
| | CTGCGCGCTCGCTCGCTCACTGAGGCCGCCCGGGCAAAGCCCGGGCGTCGGGCGACCTTTGGTCGCCCGGCCTCAGTGAGCGAGCGAGCGCGCAGAGAGGGAGTGGCCAACTCCATCACTAGGGGTTCCTTGTAGTTAATGATTA |
| | ACCCGCCATGCTACTTATCTACGGCGCGCCACGCGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACT |
| | AGTTCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACTAGTTCTAGAGCGGCCGCCAGTGTGCTGGAATTC |
| | GGCTTTTTTAGGGCTGGAAGCTACCTTTGACATCATTTCCTCTGCGAATGCATGTATAATTTCTACAGAACCTATTAGAAAGGATCACCCAGCCTCTGCTTTTGTACAACTTTCCCTTAAAAAACTGCCAATCCCACTGCTGTTT |
| | GGCCCAATAGTGAGAACTTTTTCCTGCTGCCTCTTGGTGCTTTTGCCTATGGCCCCTATTCTGCCTGCTGAAGACACTCTTGCCAGCATGGACTTAAACCCCTCCAGCTCTGACAATCCTCTTTCTCTTTTGTTTTACATGAAGG |
| | GTCTGGCAGCCAAAGCAATCACTCAAAGTTCAAACCTTATCATTTTTTGCTTTGTTCCTCTTGGCCTTGGTTTTGTACATCAGCTTTGAAAATACCATCCCAGGGTTAATGCTGGGGTTAATTTATAACTGAGAGTGCTCTAGTT |
| | CTGCAATACAGGACATGCTATAAAAATGGAAAGATGTTGCTTTCTGAGAGATCAGGTGGATTCTTGGGCATTTGCTTAAGCTTGGTACCGCCACCATGCAAATAGAGCTCTCCACCTGCTTCTTTCTGTGCCTTTTGAGGTTCTG |
| | CTTTAGTGCAACAAGAAGATACTACCTGGGTGCAGTAGAACTGTCATGGGACTATATGCAATCTGATTTAGGGGAGCTGCCTGTGGATGCAAGATTTCCACCCAGGGTGCCTAAAAGCTTTCCCTTCAACACTTCAGTTGTGTAC |
| | AAAAAGACCCTGTTTGTAGAATTCACTGATCACCTTTTCAACATTGCTAAGCCAAGGCCACCCTGGATGGGTCTGCTTGGTCCTACCATCCAGGCAGAAGTTTATGATACTGTGGTCATTACACTTAAGAACATGGCTTCCCATC |
| | CAGTGAGTCTTCATGCTGTGGGTGTATCCTACTGGAAAGCTTCTGAGGGAGCTGAATATGATGACCAGACCAGTCAAAGGGAGAAAGAAGATGACAAAGTCTTCCCAGGTGGAAGCCATACCTATGTGTGGCAGGTCCTGAAAGA |
| | GAATGGTCCAATGGCCTCTGATCCTCTGTGCCTTACCTACTCCTATCTTTCACATGTTGACCTGGTAAAAGACTTGAATTCAGGCCTCATTGGGGCCCTACTAGTTTGTAGAGAAGGGTCTCTGGCTAAGGAAAAGACTCAGACA |
| | TTGCACAAATTTATACTACTTTTTGCTGTATTTGATGAGGGGAAAAGTTGGCACTCAGAGACAAAGAACTCCTTGATGCAGGATAGAGATGCTGCTTCAGCTAGAGCATGGCCTAAAATGCACACTGTCAATGGATATGTAAACA |
| | GAAGCCTGCCAGGTCTGATTGGTTGCCACAGGAAATCTGTCTATTGGCATGTGATTGGCATGGGCACCACTCCAGAAGTGCACTCAATATTCTTAGAGGGTCACACATTTCTTGTAAGAAACCATAGACAGGCTTCCTTGGAAAT |
| | CTCACCTATAACCTTCCTTACAGCTCAAACCCTCTTGATGGACCTGGGACAGTTTCTACTGTTTTGTCATATCTCTTCCCACCAACATGATGGAATGGAAGCTTATGTCAAAGTAGACAGCTGTCCAGAGGAACCCCAACTACAG |
| | ATGAAAAATAATGAAGAAGCTGAAGACTATGATGATGATCTTACTGATAGTGAAATGGATGTAGTCAGGTTTGATGATGACAACTCTCCTTCCTTTATCCAAATTAGGTCAGTTGCTAAGAAGCATCCTAAAACTTGGGTACATT |
| | ACATTGCAGCAGAAGAGGAGGACTGGGACTATGCCCCATTAGTCCTAGCCCCTGATGATAGAAGTTATAAAAGTCAATATTTGAACAATGGACCACAGAGAATTGGTAGGAAGTACAAAAAAGTCAGATTTATGGCATACACTGA |
| | TGAGACCTTTAAGACAAGGGAAGCTATTCAGCATGAATCTGGAATCTTGGGACCTTTACTTTATGGAGAAGTTGGGGACACACTGTTGATTATATTTAAGAATCAAGCAAGCAGACCATATAACATCTACCCCCATGGAATCACT |
| | GATGTCAGGCCCTTGTATTCAAGAAGGTTACCTAAAGGTGTAAAACATTTGAAGGATTTTCCTATTCTGCCAGGTGAAATATTCAAATATAAATGGACAGTGACTGTAGAAGATGGCCCAACTAAAAGTGATCCCAGGTGCCTGA |
| | CCAGATATTACTCTAGTTTTGTCAATATGGAGAGAGATCTAGCTTCAGGACTCATTGGTCCTCTCCTCATCTGCTACAAAGAATCAGTAGATCAACAGGGAAACCAGATAATGAGTGATAAGAGGAATGTCATCCTGTTTAGTGT |
| | ATTTGATGAAAACAGAAGCTGGTACCTCACAGAAAATATACAAAGATTTCTCCCCAATCCAGCTGGGGTGCAGTTGGAGGATCCAGAATTCCAAGCAAGTAACATCATGCACAGCATCAATGGGTATGTATTTGACTCTTTGCAG |
| | TTGTCAGTATGTTTGCATGAAGTGGCATACTGGTACATTCTATCAATTGGGGCCCAGACTGATTTCCTTAGTGTCTTCTTCTCTGGTTATACATTCAAACACAAAATGGTCTATGAAGACACCCTCACCCTATTCCCATTCTCAG |
| | GGGAAACTGTCTTCATGAGTATGGAAAACCCAGGTCTATGGATTTTGGGGTGCCACAACTCAGACTTTAGGAACAGAGGCATGACTGCATTACTGAAGGTTTCAAGTTGTGATAAGAACACTGGTGATTATTATGAAGACAGTTA |
| | TGAGGATATTTCAGCATACTTGCTGAGTAAAAACAATGCAATAGAACCAAGAAGCTTCTCTCAAAACCCACCAGTCTTGAAAAGACATCAAAGAGAAATAACCAGGACTACTCTTCAGTCTGATCAAGAGGAAATTGATTATGAT |
| | GACACCATATCAGTTGAAATGAAGAAGGAAGATTTTGATATTTATGATGAGGATGAAAATCAGAGCCCAAGGAGCTTTCAAAAGAAAACCAGACACTATTTTATTGCAGCAGTAGAGAGACTCTGGGATTATGGGATGAGTTCAT |
| | CCCCCCATGTTCTAAGAAACAGGGCACAGAGTGGCAGTGTCCCACAGTTCAAGAAAGTAGTTTTCCAGGAATTTACAGATGGTTCCTTTACTCAGCCATTATACAGGGGAGAACTAAATGAACATTTGGGACTCTTAGGGCCATA |
| | TATAAGAGCTGAAGTTGAAGATAATATCATGGTAACTTTCAGGAATCAGGCCTCTAGACCATATTCCTTCTATAGTAGCCTTATTTCTTATGAGGAAGATCAGAGGCAAGGAGCAGAGCCTAGGAAAAACTTTGTTAAGCCTAAT |
| | GAAACAAAAACTTACTTTTGGAAAGTGCAACATCATATGGCACCCACCAAAGATGAGTTTGACTGCAAAGCCTGGGCCTATTTCTCTGATGTTGACTTAGAAAAAGATGTGCACTCTGGGCTGATTGGACCACTTCTGGTCTGCC |
| | ACACTAACACACTGAACCCAGCACATGGCAGACAAGTGACTGTCCAGGAATTTGCTCTGTTTTTCACCATCTTTGATGAGACAAAAAGCTGGTACTTCACTGAAAATATGGAAAGGAACTGCAGGGCTCCCTGCAATATCCAGAT |
| | GGAAGATCCAACCTTTAAAGAGAATTATAGATTCCATGCCATCAATGGGTACATAATGGATACACTACCTGGCTTAGTAATGGCTCAGGATCAAAGGATTAGGTGGTATCTGCTCAGCATGGGCTCCAATGAAAACATCCATAGC |
| | ATTCATTTCAGTGGACATGTTTTCACTGTAAGGAAAAAAGAGGAGTATAAAATGGCACTGTACAATCTCTATCCAGGAGTTTTTGAGACAGTTGAAATGTTACCATCTAAAGCAGGAATTTGGAGGGTTGAATGCCTTATTGGGG |
| | AACATCTACATGCAGGTATGAGCACACTTTTTCTGGTGTACAGCAATAAGTGTCAGACTCCACTGGGAATGGCATCTGGCCACATTAGAGATTTTCAGATTACTGCATCAGGGCAATATGGACAGTGGGCCCCAAAGCTGGCCAG |
| | ACTTCATTATAGTGGATCAATCAATGCTTGGAGCACCAAGGAGCCCTTTTCTTGGATCAAGGTGGATCTGCTTGCACCCATGATTATTCATGGGATCAAGACCCAGGGTGCCAGACAGAAGTTCAGCAGCCTCTACATCTCTCAG |
| | TTTATCATCATGTATAGCCTTGATGGAAAGAAGTGGCAGACTTATAGGGGAAATAGCACAGGAACCTTAATGGTCTTCTTTGGTAATGTTGATTCAAGTGGGATAAAACACAATATTTTTAACCCTCCAATTATAGCTAGATACA |
| | TCAGATTGCACCCCACTCATTATAGCATTAGGAGCACTCTTAGGATGGAGTTGATGGGCTGTGATTTAAATAGTTGCAGTATGCCCTTGGGAATGGAGAGTAAAGCAATATCAGATGCTCAGATTACTGCATCAAGCTACTTTAC |
| | TAATATGTTTGCTACATGGTCTCCATCAAAAGCTAGACTTCACCTCCAAGGGAGATCCAATGCTTGGAGACCTCAGGTGAATAATCCAAAAGAGTGGCTGCAAGTGGACTTCCAGAAGACTATGAAAGTCACAGGGGTAACAACC |
| | CAGGGAGTAAAATCTCTGCTTACCAGCATGTATGTCAAGGAGTTCCTCATCTCCAGCTCACAAGATGGGCATCAGTGGACTCTCTTTTTTCAGAATGGGAAAGTAAAGGTTTTTCAGGGAAATCAAGACTCCTTCACACCAGTGG |
| | TGAACTCTTTGGACCCACCTTTACTGACTAGATACCTTAGGATTCACCCACAGAGTTGGGTGCACCAGATAGCTCTGAGAATGGAGGTTCTGGGCTGTGAGGCCCAGGACCTCTACTGATTTAAATCTCGAGCCGGGCGGAGTGT |
| | GTTAGTCTCTCCAGAGGGAGGCTGGTTCCCCAGGGAAGCAGAGCCTGTGTGCGGGCAGCAGCTGTGTGCGGGCCTGGGGGTTGTTAAGTGCAATTATTTTTAATAAAAGGGGCATTTGGAAAAAAAAAAAAAAGGTAGCAGTCGA |
| | CAGATGAATTCTGCAGATCTGTGGCTTCTAGCTGCCCGGGTGGCATCCCTGTGACCCCTCCCCAGTGCCTCTCCTGGCCCTGGAAGTTGCCACTCCAGTGCCCACCAGCCTTGTCCTAATAAAATTAAGTTGCATCATTTTGTCT |
| | GACTAGGTGTCCTTCTATAATATTATGGGGTGGAGGGGGGTGGTATGGAGCAAGGGGCAAGTTGGGAAGACAACCTGTAGGGCCTGCGGGGTCTATTGGGAACCAAGCTGGAGTGCAGTGGCACAATCTTGGCTCACTGCAATCT |
| | CCGCCTCCTGGGTTCAAGCGATTCTCCTGCCTCAGCCTCCCGAGTTGTTGGGATTCCAGGCATGCATGACCAGGCTCAGCTAATTTTTGTTTTTTTGGTAGAGACGGGGTTTCACCATATTGGCCAGGCTGGTCTCCAACTCCTA |
| | ATCTCAGGTGATCTACCCACCTTGGCCTCCCAAATTGCTGGGATTACAGGCGTGAACCACTGCTCCCTTCCCTGTCCTTCTGATTTTAAAAAAAAGAGAAATGTTCTGGCACCTGCACTTGCACTGGGGACAGCCTATTTTGCTA |
| | GTTTGTTTTGTTTCGTTTTGTTTTGATGGAGAGCGTATGTTGTTTAAACGCGGCCGCGTAGATAAGTAGCATGGCGGGTTAATCATTAACTACAAGGAACCCCTAGTGATGGAGTTGGCCACTCCCTCTCTGCGCGCTCGCTCGC |
| | TCACTGAGGCCGGGCGACCAAAGGTCGCCCGACGCCCGGGCTTTGCCCGGGCGGCCTCAGTGAGCGAGCGAGCGCGCAG |
| 54 | >nucleotide of 201 signal peptide |
| | ATGGAGTTCGGGCTGAGCTGGGTCTTTCTGGTGGCCCTGCTGAAGGGAGTCCAGTGCGAG |
| 55 | >nucleotide of wt IL2 signal peptide |
| | ATGTACCGTATGCAGCTGCTGTCCTGCATCGCACTGTCACTGGCACTGGTTACCAACTCA |
| 56 | >nucleotide of mut IL2 signal peptide |
| | ATGTACCGTATGCAGCTGCTGCTGCTGATCGCACTGTCACTGGCACTGGTTACCAACTCA |
| 57 | >nucleotide of 2AM signal peptide |
| | ATGGGGAAGAACAAACTCCTTCATCCAAGTCTGGTTCTTCTCCTCTTGGTCCTCCTGCCCACAGATGCC |
| 58 | >nucleotide of PZP signal peptide |
| | ATGAGAAAAGACAGACTTCTTCATTTATGTCTTGTGCTACTTCTTATCCTGCTTTCTGCCAGTGACTCAAACTCT |
| 59 | |
| 60 | >201 signal peptide |
| | MEFGLSWVFLVALLKGVQCE |
| 61 | >wt IL2 signal peptide |
| | MYRMQLLSCIALSLALVTNS |
| 62 | >mut IL2 signal peptide |
| | MYRMQLLLLIALSLALVINS |
| 63 | >A2M signal peptide |
| | MGKNKLLHPSLVLLLLVLLPTDA |
| 64 | >PZP signal peptide |
| | MRKDRLLHLCLVLLLILLSASDSNS |
| 65 | >artificial signal peptide |
| | MWWRLWWLLLLLLLLWPMVWA |
| 66 | >cathpetsin L signal peptide |
| | LLLLAVLCLGT |
| 67 | >nucleotide of hAAT signal peptide |
| | ATGCCGTCTTCTGTCTCGTGGGGCATCCTCCTGCTGGCAGGCCTGTGCTGCCTGGTCCCTGTCTCCCTGGCT |
| 68 | >FIBrat Signal Peptide |
| | MLRGPGPGRLLLLAVLCLGTSVRCTETGKSKR |
| 69 | >FIBrat Signal Peptide |
| | MLRGPGPGRLLLLAVLCLGTSVRCTETGKSKRLALQIV |
| 70 | >FN1 human signal peptide |
| | MLRGPGPGLLLLAVQCLGTAVPSTGA |
| 71 | >FN1 x. leavis signal peptide |
| | MRRGALTGLLLVLCLSVVLRAAPSATSKKRR |
| 72 | >hIGF2 signal peptide |
| | ATGGGAATCCCAATGGGGAAGTCGATGCTGGTGCTTCTCACCTTCTTGGCCTTCGCCTCGTGCTGCATTGCT |
| 73 | >FN1 rat signal peptide |
| | ATGCTCAGGGGTCCGGGACCCGGGCGGCTGCTGCTGCTAGCAGTCCTGTGCCTGGGGACATCGGTGCGCTGCACCGAAACCGGGAAGAGCAAGAGG |
| 74 | >FN1 rat signal peptide |
| | ATG CTC AGG GGT CCG GGA CCC GGG CGG CTG CTG CTG CTA GCA GTC CTG TGC CTG GGG ACA TCG GTG CGC TGC ACC GAA ACC GGG AAG AGC AAG AGG CAG GCT CAG CAA |
| | ATC GTG-3′. |
| 75 | >FN1 human signal peptide |
| | ATGCTTAGGGGTCCGGGGCCCGGGCTGCTGCTGCTGGCCGTCCAGTGCCTGGGGACAGCGGTGCCCTCCACGGGAGCC |
| 76 | >FN1 x. leavis signal peptide |
| | ATG CGC CGG GGG GCC CTG ACC GGG CTG CTC CTG GTC CTG TGC CTG AGT GTT GTG CTA CGT GCA GCC CCC TCT GCA ACA AGC AAG AAG CGC AGG |
| 77 | >preprocathepsin L signal peptide (rat) |
| | MTPLLLLAVLCLGTALA |
| 78 | >prepro-alpha 2 type collagen signal peptide (homo sapiens) |
| | MLSFVDTRTLLLLAVTLCLATC |
| 79 | >SEQ 79 CTGCGCGCTCGCTCGCTCACTGAGGCCGCCCGGGCAAAGCCCGGGCGTCGGGCGACCTTTGGTCGCCCGGCCTCAGTGAGCGAGCGAGCGCGCAGAGAGGGAGTGGCCAACTCCATCACTAGGGGTTCCT |
| 80 | >SEQ 80 AGGAACCCCTAGTGATGGAGTTGGCCACTCCCTCTCTGCGCGCTCGCTCGCTCACTGAGGCCGGGCGACCAAAGGTCGCCCGACGCCCGGGCTTTGCCCGGGCGGCCTCAGTGAGCGAGCGAGCGCGCAG |
| 81 | >SEQ 81 CTGCGCGCTCGCTCGCTCACTGAGGCCGCCCGGGCAAAGCCCGGGCGTCGGGCGACCTTTGGTCGCCCGGCCTCAGTGAGCGAGCGAGCGCGCAGAGAGGGAGTGGCCAACTCCATCACTAGGG |
| 82 | >SEQ 82 |
| | AGGAACCCCTAGTGATGGAGTTGGCCACTCCCTCTCTGCGCGCTCGCTCGCTCACTGAGGCCGGGCGACCAAAGGTCGCCCGACGCCCGGGCTTTGCCCGGGCGGCCTCAGTGAGCGAGCGAGCGCGCAG |
| 83 | >MH-257 ITR |
| | AGGAACCCCTAGTGATGGAGTTGGCCACTCCCTCTCTGCGCGCTCGCTCGCTCACTGAGGCAATTTGATAAAAATCGTCAAATTATAAACAGGCTTTGCCTGTTTAGCCTCAGTGAGCGAGCGAGCGCGCAGAGAGGGAGTGGCC |
| | AACTCCATCACTAGGGGTTCCT |
| 84 | >MH-258 ITR |
| | AGGAACCCCTAGTGATGGAGTTGGCCACTCCCTCTCTGCGCGCTCGCTCGCTCACTGAGGGATAAAAATCCAGGCTTTGCCTGCCTCAGTGAGCGAGCGAGCGCGCAGAGAGGGAGTGGCCAACTCCATCACTAGGGGTTCCT |
| 85 | >MH Delta |
| | 258AGGAACCCCTAGTGATGGAGTTGGCCACTCCCTCTCTGCGCGCTCGCTCGCTCACTGAGGGATAAAAATCCAGGCTTTGCCTGCCTCAGTGAGCGAGCGAGCGCGCAGAGAGGGAGTGGCCAACTCCATCACTAGGGGTTCC |
| | T |
| 100 | >MH Telomere-1 ITR |
| | AGGAACCCCTAGTGATGGAGTTGGCCACTCCCTCTCTGGGATTGGGATTGCGCGCTCGCTCGCGGGATTGGGATTGGGATTGGGATTGGGATTGGGATTGATAAAAATCAATCCCAATCCCAATCCCAATCCCAATCCCAATCCC |
| | GCGAGCGAGCGCGCAATCCCAATCCCAGAGAGGGAGTGGCCAACTCCATCACTAGGGGTTCCT |
| 101 | >MH Telomere-2 ITR |
| | AGGAACCCCTAGTGATGGAGTTGGCCACTCCCTCTCTGCGCGCTCGCTCGCTCGGGATTGGGATTGGGATTGGGATTGGGATTGGGATTGATAAAAATCAATCCCAATCCCAATCCCAATCCCAATCCCAATCCCGCGAGCGAGC |
| | GCGCAGGAGAGGGAGTGGCCAACTCCATCACTAGGGGTTCCTAAGCTTATTATA |
| 102 | >MH PolII 258 ITR |
| | AGGAACCCCTAGTGATGGAGTTGGCCACTCCCTCTCTGCGCGCTCGCTCGCTCACTGAGGGCGCCTATAAAGATAAAAATCCAGGCTTTGCCTGCCTCAGTTAGCGAGCGAGCGCGCAGAGAGGGAGTGGCCAACTCCATCACTA |
| | GGGGTTCCT |
| 103 | >MH 258 Delta D conservative |
| | CTAGTGATGGAGTTGGCCACTCCCTCTCTGCGCGCTCGCTCGCTCACTGAGGGATAAAAATCCAGGCTTTGCCTGCCTCAGTGAGCGAGCGAGCGCGCAGAGAGGGAGTGGCCAACTCCATCACTAG |
| 104 | >5′ AAV2-ITR |
| | TTGGCCACTCCCTCTCTGCGCGCTCGCTCGCTCACTGAGGCCGGGCGACCAAAGGTCGCCCGACGCCCGGGCTTTGCCCGGGCGGCCTCAGTGAGCGAGCGAGCGCGCAGAGAGGGAGTGGCCAACTCCATCACTAGGGGTTCCT |
| 105 | >3′ AAV2 ITR |
| | AGGAACCCCTAGTGATGGAGTTGGCCACTCCCTCTCTGCGCGCTCGCTCGCTCACTGAGGCCGGGCGACCAAAGGTCGCCCGACGCCCGGGCTTTGCCCGGGCGGCCTCAGTGAGCGAGCGAGCGCGCAGAGAGGGAGTGGCCAA |
| 448 | >ITR (145 bp) |
| | AGGAACCCCTAGTGATGGAGTTGGCCACTCCCTCTCTGCGCGCTCGCTCGCTCACTGAGGCCGGGCGACCAAAGGTCGCCCGACGCCCGGGCTTTGCCCGGGCGGCCTCAGTGAGCGAGCGAGCGCGCAGAGAGGGAGTGGCCAA |
| 449 | >ITR (lacking 9 bp) |
| | AGGAACCCCTAGTGATGGAGTTGGCCACTCCCTCTCTGCGCGCTCGCTCGCTCACTGAGGCCGGGCGACCAAAGGTCGCCCGACGCCCGGGCTTTGCCCGGGCGGCCTCAGTGAGCGAGCGAGCGCGCAGAGAGGG |
| 450 | >CRE0052 |
| | GGGCATATAAAACAGGGGCAAGGCACAGACTCATAGCAGAGCAATCACCACCAAGCCTGGAATAACTGCAGCCACC |
| 451 | |
| 97 | >LSP |
| | TCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACTAGTTCTAGGAGTTAATTTTTAAAAAGCAGTCAAAAG |
| | TCCAAGTGCCCTTGCGAGCATTTACTCTCTCTGTTTGCTCTGGTTAATAATCTCAGGAGCACAAACATTCCTTACTAGTTCTAGAGCGGCCGCCAGTGTGCTGGAATTCGGCTTTTTTAGGGCTGGAAGCTACCTTTGACATCAT |
| | TTCCTCTGCGAATGCATGTATAATTTCTACAGAACCTATTAGAAAGGATCACCCAGCCTCTGCTTTTGTACAACTTTCCCTTAAAAAACTGCCAATCCCACTGCTGTTTGGCCCAATAGTGAGAACTTTTTCCTGCTGCCTCTTG |
| | GTGCTTTTGCCTATGGCCCCTATTCTGCCTGCTGAAGACACTCTTGCCAGCATGGACTTAAACCCCTCCAGCTCTGACAATCCTCTTTCTCTTTTGTTTTACATGAAGGGTCTGGCAGCCAAAGCAATCACTCAAAGTTCAAACC |
| | TTATCATTTTTTGCTTTGTTCCTCTTGGCCTTGGTTTTGTACATCAGCTTTGAAAATACCATCCCAGGGTTAATGCTGGGGTTAATTTATAACTGAGAGTGCTCTAGTTCTGCAATACAGGACATGCTATAAAAATGGAAAGATG |
| | TTGCTTTCTGAGAGAT |
| 98 | >M2 LSP |
| | TCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACTAGTTCTAGGAGTTAATTTTTAAAAAGCAGTCAAAAG |
| | TCCAAGTGCCCTTGCGAGCATTTACTCTCTCTGTTTGCTCTGGTTAATAATCTCAGGAGCACAAACATTCCTTACTAGTTCTAGAGCGGCCGCCAGTGTGCTGGAATTCGGCTTTTTTAGGGCTGGAAGCTACCTTTGACATCAT |
| | TTCCTCTGCGAATGCATGTATAATTTCTACAGAACCTATTAGAAAGGATCACCCAGCCTCTGCTTTTGTACAACTTTCCCTTAAAAAACTGCCAATCCCACTGCTGTTTGGCCCAATAGTGAGAACTTTTTCCTGCTGCCTCTTG |
| | GTGCTTTTGCCTATGGCCCCTATTCTGCCTGCTGAAGACACTCTTGCCAGCATGGACTTAAACCCCTCCAGCTCTGACAATCCTCTTTCTCTTTTGTTTTACATGAAGGGTCTGGCAGCCAAAGCAATCACTCAAAGTTCAAACC |
| | TTATCATTTTTTGCTTTGTTCCTCTTGGCCTTGGTTTTGTACATCAGCTGTGAAAATACCATCCCAGGGTATTTATAGCTGGGGTTAATTTATAACTGAGAGTGCTCTAGTTCTGCAATACAGGACATGCTATAAAAATGGAAAG |
| | ATGTTGCTTTCTGAGAGATCAGCTTACATGTG |
| 99 | >M3 LSP |
| | TCTAGGAGTTAATTTTTAAAAAGCAGTCAAAAGTCCAAGTGCCCTTGCGAGCATTTACTCTCTCTGTTTGCTCTGGTTAATAATCTCAGGAGCACAAACATTCCTTACTAGTTCTAGGAGTTAATTTTTAAAAAGCAGTCAAAAG |
| | TCCAAGTGCCCTTGCGAGCATTTACTCTCTCTGTTTGCTCTGGTTAATAATCTCAGGAGCACAAACATTCCTTACTAGTTCTAGAGCGGCCGCCAGTGTGCTGGAATTCGGCTTTTTTAGGGCTGGAAGCTACCTTTGACATCAT |
| | TTCCTCTGCGAATGCATGTATAATTTCTACAGAACCTATTAGAAAGGATCACCCAGCCTCTGCTTTTGTACAACTTTCCCTTAAAAAACTGCCAATCCCACTGCTGTTTGGCCCAATAGTGAGAACTTTTTCCTGCTGCCTCTTG |
| | GTGCTTTTGCCTATGGCCCCTATTCTGCCTGCTGAAGACACTCTTGCCAGCATGGACTTAAACCCCTCCAGCTCTGACAATCCTCTTTCTCTTTTGTTTTACATGAAGGGTCTGGCAGCCAAAGCAATCACTCAAAGTTCAAACC |
| | TTATCATTTTTTGCTTTGTTCCTCTTGGCCTTGGTTTTGTACATCAGCTTTGAAAATACCATCCCAGGGTTAATGCTGGGGTTAATTTATAACTGAGAGTGCTCTAGTTCTGCAATACAGGACATGCTATAAAAATGGAAAGATG |
| | TTGCTTTCTGAGAGATCAGCTTACATGTG |
| 86 | >CRM_SP0412 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCCCCTGTTCAAACATGTCCTAATACTCTGTCTCTGCAAGGGTCATC |
| | AGTAGTTTTCCATCTTACTCAACATCCTCCCAGTG |
| 87 | >CRM_SP0239 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTGAGGTTAATTTTTAAAAAGCAGTCAAAAGTCCAAGTGGCCCTT |
| | GGCAGCATTTACTCTCTCTGTTTGCTCTGGTTAATAATCTCAGGAGCACAAACATTCCGGCCCGGGAGGCGCCCTTTGGACCTTTTGCAATCCTGGCGCACTGAACCCTTGACCCCTGCCCTGCAGCCCCCGCAGCTTGCTGTTT |
| | GCCCACTCTATTTGCCCAGCCCCAGCCCTGGAGAGTCCTTTAGCAGGGCAAAGTGCAACATAGGCAGACCTTAAGGGATGACTCAGTAACAGATAAGCTTTGTGTGCCTGCA |
| 88 | >CRM_SP0265 (CRM_LVR_131_A1) |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTGTACCGGCCCGGGAGGCGCCCTTTGGACCTTTTGCAATCCTGGC |
| | GCACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAACATAGGCAGACCTTAAGGGATGACTCAGTAACAGATAAG |
| | CTTTGTGTGCCTGCA |
| 89 | >CRM_SP0240 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTG |
| 90 | >CRM_SP0246 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGCACTG |
| | AACCCTTGACCCCTGCCCTGCAGCCCCCGCAGCTTGCTGTTTGCCCACTCTATTTGCCCAGCCCCAG |
| 91 | >SP0412 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCCCCTGTTCAAACATGTCCTAATACTCTGTCTCTGCAAGGGTCATC |
| | AGTAGTTTTCCATCTTACTCAACATCCTCCCAGTGAGTCATATGTTTGCTCACTGAAGGTTACTAGTTAACAGGCATCCCTTAAACAGGATATAAAAGGACTTCAGCAGGACTGCTCGAAACATCCCACTCAGCCACC |
| 92 | >SP0422 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCCCCTGTTCAAACATGTCCTAATACTCTGTCTCTGCAAGGGTCATC |
| | AGTAGTTTTCCATCTTACTCAACATCCTCCCAGTGAGTCATATGTTTGCTCACTGAAGGTTACTAGTTAACAGGCATCCCTTAAACAGGATATAAAAGGACTTCAGCAGGACTGCTCGAAACATCCCACTTCAGATCGCCTGGAG |
| | ACGCCATCCACGCTGTTTTGACCTCCATAGAAGACACCGGGACCGATCCAGCCTCCGCGGCCGGGAACGGTGCATTGGAACGCGGATTCCCCGTGCCAAGAGTGACGTAAGTACCGCCTATAGACTCTATAGGCACACCCCTTTG |
| | GCTCTTATGCATGAACGGTGGAGGGCAGTGTAGTCTGAGCAGTACTCGTTGCTGCCGCGCGCGCCACCAGACATAATAGCTGACAGACTAACAGACTGTTCCTTTCCATGGGTCTTTTCTGCAGGCCACC |
| 93 | >SP0239 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTGAGGTTAATTTTTAAAAAGCAGTCAAAAGTCCAAGTGGCCCTT |
| | GGCAGCATTTACTCTCTCTGTTTGCTCTGGTTAATAATCTCAGGAGCACAAACATTCCGGCCCGGGAGGCGCCCTTTGGACCTTTTGCAATCCTGGCGCACTGAACCCTTGACCCCTGCCCTGCAGCCCCCGCAGCTTGCTGTTT |
| | GCCCACTCTATTTGCCCAGCCCCAGCCCTGGAGAGTCCTTTAGCAGGGCAAAGTGCAACATAGGCAGACCTTAAGGGATGACTCAGTAACAGATAAGCTTTGTGTGCCTGCAGGGCATATAAAACAGGGGCAAGGCACAGACTCA |
| | TAGCAGAGCAATCACCACCAAGCCTGGAATAACTGCAGCCACC |
| 94 | >SP0265 (LVR_SP131_A1) |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTGTACCGGCCCGGGAGGCGCCCTTTGGACCTTTTGCAATCCTGGC |
| | GCACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAACATAGGCAGACCTTAAGGGATGACTCAGTAACAGATAAG |
| | CTTTGTGTGCCTGCAGGGCATATAAAACAGGGGCAAGGCACAGACTCATAGCAGAGCAATCACCACCAAGCCTGGAATAACTGCAGCCACC |
| 95 | >SP0240 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTGCCGATGACCTAATGATTCTGAGCTTGGCAAAGGTCTTATCTC |
| | CCAGCTCGCCCAGGCCCAGTGTTCCAGGAATGTGACCTTTGCTGCAGCAGCCGCTGGAGGGGGCAGAGGGGATGGGCTGGAGGTTGAGCAAACAGAGCAGCAGAAAAGGCAGTTCCTCTTCTCCAGTGCCCTCCTTCCCTGTCTC |
| | TGCCTCTCCCTCCCTTCCTCAGGCATCAGAGCGGAGACTTCAGGGAGACCAGAGCCCAGCTTGCCAGGCACTGAGCTAGAAGCCCTGCC |
| 96 | >SP0246 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGCACTG |
| | AACCCTTGACCCCTGCCCTGCAGCCCCCGCAGCTTGCTGTTTGCCCACTCTATTTGCCCAGCCCCAGGGGCATATAAAACAGGGGCAAGGCACAGACTCATAGCAGAGCAATCACCACCAAGCCTGGAATAACTGCAGCCACC |
| 144 | >SP0131 |
| | GGCCCGGGAGGCGCCCTTTGGACCTTTTGCAATCCTGGCGCACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAA |
| | CATAGGCAGACCTTAAGGGATGACTCAGTAACAGATAAGCTTTGTGTGCCTGCAGGGCATATAAAACAGGGGCAAGGCACAGACTCATAGCAGAGCAATCACCACCAAGCCTGGAATAACTGCAGCCACC (SEQ ID NO: |
| | 144), |
| 145 | >CMV-IE 5′ UTR |
| | TCAGATCGCCTGGAGACGCCATCCACGCTGTTTTGACCTCCATAGAAGACACCGGGACCGATCCAGCCTCCGCGGCCGGGAACGGTGCATTGGAACGCGGATTCCCCGTGCCAAGAGTGACGTAAGTACCGCCTATAGACTCTAT |
| | AGGCACACCCCTTTGGCTCTTATGCATGAACGGTGGAGGGCAGTGTAGTCTGAGCAGTACTCGTTGCTGCCGCGCGCGCCACCAGACATAATAGCTGACAGACTAACAGACTGTTCCTTTCCATGGGTCTTTTCTGCAG (SEQ |
| | ID NO: 145), |
| 146 | >SP0265-UTR |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTGTACCGGCCCGGGAGGCGCCCTTTGGACCTTTTGCAATCCTGGC |
| | GCACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAACATAGGCAGACCTTAAGGGATGACTCAGTAACAGATAAG |
| | CTTTGTGTGCCTGCAGGGCATATAAAACAGGGGCAAGGCACAGACTCATAGCAGAGTCAGATCGCCTGGAGACGCCATCCACGCTGTTTTGACCTCCATAGAAGACACCGGGACCGATCCAGCCTCCGCGGCCGGGAACGGTGCA |
| | TTGGAACGCGGATTCCCCGTGCCAAGAGTGACGTAAGTACCGCCTATAGACTCTATAGGCACACCCCTTTGGCTCTTATGCATGAACGGTGGAGGGCAGTGTAGTCTGAGCAGTACTCGTTGCTGCCGCGCGCGCCACCAGACAT |
| | AATAGCTGACAGACTAACAGACTGTTCCTTTCCATGGGTCTTTTCTGCAGGCCACC (SEQ ID NO: 147 |
| 147 | >SP0239-UTR |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTGAGGTTAATTTTTAAAAAGCAGTCAAAAGTCCAAGTGGCCCTT |
| | GGCAGCATTTACTCTCTCTGTTTGCTCTGGTTAATAATCTCAGGAGCACAAACATTCCGGCCCGGGAGGCGCCCTTTGGACCTTTTGCAATCCTGGCGCACTGAACCCTTGACCCCTGCCCTGCAGCCCCCGCAGCTTGCTGTTT |
| | GCCCACTCTATTTGCCCAGCCCCAGCCCTGGAGAGTCCTTTAGCAGGGCAAAGTGCAACATAGGCAGACCTTAAGGGATGACTCAGTAACAGATAAGCTTTGTGTGCCTGCAGGGCATATAAAACAGGGGCAAGGCACAGACTCA |
| | TAGCAGAGTCAGATCGCCTGGAGACGCCATCCACGCTGTTTTGACCTCCATAGAAGACACCGGGACCGATCCAGCCTCCGCGGCCGGGAACGGTGCATTGGAACGCGGATTCCCCGTGCCAAGAGTGACGTAAGTACCGCCTATA |
| | GACTCTATAGGCACACCCCTTTGGCTCTTATGCATGAACGGTGGAGGGCAGTGTAGTCTGAGCAGTACTCGTTGCTGCCGCGCGCGCCACCAGACATAATAGCTGACAGACTAACAGACTGTTCCTTTCCATGGGTCTTTTCTGC |
| | AGGCCACC (SEQ ID NO: 147), |
| 148 | >SP0240-UTR |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTGCCGATGACCTAATGATTCTGAGCTTGGCAAAGGTCTTATCTC |
| | CCAGCTCGCCCAGGCCCAGTGTTCCAGGAATGTGACCTTTGCTGCAGCAGCCGCTGGAGGGGGCAGAGGGGATGGGCTGGAGGTTGAGCAAACAGAGCAGCAGAAAAGGCAGTTCCTCTTCTCCAGTGCCCTCCTTCCCTGTCTC |
| | TGCCTCTCCCTCCCTTCCTCAGGCATCAGAGCGGAGACTTCAGGGAGACCAGAGCCCAGCTTGCCAGGCACTGTCAGATCGCCTGGAGACGCCATCCACGCTGTTTTGACCTCCATAGAAGACACCGGGACCGATCCAGCCTCCG |
| | CGGCCGGGAACGGTGCATTGGAACGCGGATTCCCCGTGCCAAGAGTGACGTAAGTACCGCCTATAGACTCTATAGGCACACCCCTTTGGCTCTTATGCATGAACGGTGGAGGGCAGTGTAGTCTGAGCAGTACTCGTTGCTGCCG |
| | CGCGCGCCACCAGACATAATAGCTGACAGACTAACAGACTGTTCCTTTCCATGGGTCTTTTCTGCAGGCCACC (SEQ ID NO: 148 |
| 149 | >SP0246-UTR |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGCACTG |
| | AACCCTTGACCCCTGCCCTGCAGCCCCCGCAGCTTGCTGTTTGCCCACTCTATTTGCCCAGCCCCAGGGGCATATAAAACAGGGGCAAGGCACAGACTCATAGCAGAGTCAGATCGCCTGGAGACGCCATCCACGCTGTTTTGAC |
| | CTCCATAGAAGACACCGGGACCGATCCAGCCTCCGCGGCCGGGAACGGTGCATTGGAACGCGGATTCCCCGTGCCAAGAGTGACGTAAGTACCGCCTATAGACTCTATAGGCACACCCCTTTGGCTCTTATGCATGAACGGTGGA |
| | GGGCAGTGTAGTCTGAGCAGTACTCGTTGCTGCCGCGCGCGCCACCAGACATAATAGCTGACAGACTAACAGACTGTTCCTTTCCATGGGTCTTTTCTGCAGGCCACC (SEQ ID NO: 149), |
| 150 | >SP0131_A1-UTR |
| | GGCCCGGGAGGCGCCCTTTGGACCTTTTGCAATCCTGGCGCACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAA |
| | CATAGGCAGACCTTAAGGGATGACTCAGTAACAGATAAGCTTTGTGTGCCTGCAGGGCATATAAAACAGGGGCAAGGCACAGACTCATAGCAGAGTCAGATCGCCTGGAGACGCCATCCACGCTGTTTTGACCTCCATAGAAGAC |
| | ACCGGGACCGATCCAGCCTCCGCGGCCGGGAACGGTGCATTGGAACGCGGATTCCCCGTGCCAAGAGTGACGTAAGTACCGCCTATAGACTCTATAGGCACACCCCTTTGGCTCTTATGCATGAACGGTGGAGGGCAGTGTAGTC |
| | TGAGCAGTACTCGTTGCTGCCGCGCGCGCCACCAGACATAATAGCTGACAGACTAACAGACTGTTCCTTTCCATGGGTCTTTTCTGCAGGCCACC (SEQ ID NO: 150), |
| 154 | >LVR412_askbioEU |
| | GCCATTCGCCATTCAGGCTGCGCAACTGTTGGGAAGGGCGATCGGTGCGGGCCTCTTCGCTATTACGCCAGCTGGCGAAAGGGGGATGTGCTGCAAGGCGATTAAGTTGGGTAACGCCAGGGTTTTCCCAGTCACGACGTTGTAA |
| | AACGACGGCCAGTGAATTCCTCGAGATTTAAATGACGTTGGCCACTCCCTCTCTGCGCGCTCGCTCGCTCACTGAGGCCGGGCGACCAAAGGTCGCCCGACGCCCGGGCTTTGCCCGGGCGGCCTCAGTGAGCGAGCGAGCGCGC |
| | AGAGAGGGAGTGGCCAACTCCATCACTAGGGGTTCCTGAGTTTAAACTTCGTCGAGGCCGCACGCGTAGGTTAATTTTTAAAAAGCAGTCAAAAGTCCAAGTGGCCCTTGGCAGCATTTACTCTCTCTGTTTGCTCTGGTTAATA |
| | ATCTCAGGAGCACAAACATTCCCCCTGTTCAAACATGTCCTAATACTCTGTCTCTGCAAGGGTCATCAGTAGTTTTCCATCTTACTCAACATCCTCCCAGTGAGTCATATGTTTGCTCACTGAAGGTTACTAGTTAACAGGCATC |
| | CCTTAAACAGGATATAAAAGGACTTCAGCAGGACTGCTCGAAACATCCCACTCAGCCACCATGGGAGTGAGGCACCCGCCCTGCTCCCACCGGCTCCTGGCCGTCTGCGCCCTCGTGTCCTTGGCAACCGCTGCACTCCTGGGGC |
| | ACATCCTACTCCATGATTTCCTGCTGGTTCCCCGAGAGCTGAGTGGCTCCTCCCCAGTCCTGGAGGAGACTCACCCAGCTCACCAGCAGGGAGCCAGCAGACCAGGGCCCCGGGATGCCCAGGCACACCCCGGCCGTCCCAGAGC |
| | AGTGCCCACACAGTGCGACGTCCCCCCCAACAGCCGCTTCGATTGCGCCCCTGACAAGGCCATCACCCAGGAACAGTGCGAGGCCCGCGGCTGCTGCTACATCCCTGCAAAGCAGGGGCTGCAGGGAGCCCAGATGGGGCAGCCC |
| | TGGTGCTTCTTCCCACCCAGCTACCCCAGCTACAAGCTGGAGAACCTGAGCTCCTCTGAAATGGGCTACACGGCCACCCTGACCCGTACCACCCCCACCTTCTTCCCCAAGGACATCCTGACCCTGCGGCTGGACGTGATGATGG |
| | AGACTGAGAACCGCCTCCACTTCACGATCAAAGATCCAGCTAACAGGCGCTACGAGGTGCCCTTGGAGACCCCGCGTGTCCACAGCCGGGCACCGTCCCCACTCTACAGCGTGGAGTTCTCCGAGGAGCCCTTCGGGGTGATCGT |
| | GCACCGGCAGCTGGACGGCCGCGTGCTGCTGAACACGACGGTGGCGCCCCTGTTCTTTGCGGACCAGTTCCTTCAGCTGTCCACCTCGCTGCCCTCGCAGTATATCACAGGCCTCGCCGAGCACCTCAGTCCCCTGATGCTCAGC |
| | ACCAGCTGGACCAGGATCACCCTGTGGAACCGGGACCTTGCGCCCACGCCCGGTGCGAACCTCTACGGGTCTCACCCTTTCTACCTGGCGCTGGAGGACGGCGGGTCGGCACACGGGGTGTTCCTGCTAAACAGCAATGCCATGG |
| | ATGTGGTCCTGCAGCCGAGCCCTGCCCTTAGCTGGAGGTCGACAGGTGGGATCCTGGATGTCTACATCTTCCTGGGCCCAGAGCCCAAGAGCGTGGTGCAGCAGTACCTGGACGTTGTGGGATACCCGTTCATGCCGCCATACTG |
| | GGGCCTGGGCTTCCACCTGTGCCGCTGGGGCTACTCCTCCACCGCTATCACCCGCCAGGTGGTGGAGAACATGACCAGGGCCCACTTCCCCCTGGACGTCCAATGGAACGACCTGGACTACATGGACTCCCGGAGGGACTTCACG |
| | TTCAACAAGGATGGCTTCCGGGACTTCCCGGCCATGGTGCAGGAGCTGCACCAGGGCGGCCGGCGCTACATGATGATCGTGGATCCTGCCATCAGCAGCTCGGGCCCTGCCGGGAGCTACAGGCCCTACGACGAGGGTCTGCGGA |
| | GGGGGGTTTTCATCACCAACGAGACCGGCCAGCCGCTGATTGGGAAGGTATGGCCCGGGTCCACTGCCTTCCCCGACTTCACCAACCCCACAGCCCTGGCCTGGTGGGAGGACATGGTGGCTGAGTTCCATGACCAGGTGCCCTT |
| | CGACGGCATGTGGATTGACATGAACGAGCCTTCCAACTTCATCAGAGGCTCTGAGGACGGCTGCCCCAACAATGAGCTGGAGAACCCACCCTACGTGCCTGGGGTGGTTGGGGGGACCCTCCAGGCGGCCACCATCTGTGCCTCC |
| | AGCCACCAGTTTCTCTCCACACACTACAACCTGCACAACCTCTACGGCCTGACCGAAGCCATCGCCTCCCACAGGGCGCTGGTGAAGGCTCGGGGGACACGCCCATTTGTGATCTCCCGCTCGACCTTTGCTGGCCACGGCCGAT |
| | ACGCCGGCCACTGGACGGGGGACGTGTGGAGCTCCTGGGAGCAGCTCGCCTCCTCCGTGCCAGAAATCCTGCAGTTTAACCTGCTGGGGGTGCCTCTGGTCGGGGCCGACGTCTGCGGCTTCCTGGGCAACACCTCAGAGGAGCT |
| | GTGTGTGCGCTGGACCCAGCTGGGGGCCTTCTACCCCTTCATGCGGAACCACAACAGCCTGCTCAGTCTGCCCCAGGAGCCGTACAGCTTCAGCGAGCCGGCCCAGCAGGCCATGAGGAAGGCCCTCACCCTGCGCTACGCACTC |
| | CTCCCCCACCTCTACACACTGTTCCACCAGGCCCACGTCGCGGGGGAGACCGTGGCCCGGCCCCTCTTCCTGGAGTTCCCCAAGGACTCTAGCACCTGGACTGTGGACCACCAGCTCCTGTGGGGGGAGGCCCTGCTCATCACCC |
| | CAGTGCTCCAGGCCGGGAAGGCCGAAGTGACTGGCTACTTCCCCTTGGGCACATGGTACGACCTGCAGACGGTGCCAATAGAGGCCCTTGGCAGCCTCCCACCCCCACCTGCAGCTCCCCGTGAGCCAGCCATCCACAGCGAGGG |
| | GCAGTGGGTGACGCTGCCGGCCCCCCTGGACACCATCAACGTCCACCTCCGGGCTGGGTACATCATCCCCCTGCAGGGCCCTGGCCTCACAACCACAGAGTCCCGCCAGCAGCCCATGGCCCTGGCTGTGGCCCTGACCAAGGGT |
| | GGAGAGGCCCGAGGGGAGCTGTTCTGGGACGATGGAGAGAGCCTGGAAGTGCTGGAGCGAGGGGCCTACACACAGGTCATCTTCCTGGCCAGGAATAACACGATCGTGAATGAGCTGGTACGTGTGACCAGTGAGGGAGCTGGCC |
| | TGCAGCTGCAGAAGGTGACTGTCCTGGGCGTGGCCACGGCGCCCCAGCAGGTCCTCTCCAACGGTGTCCCTGTCTCCAACTTCACCTACAGCCCCGACACCAAGGTCCTGGACATCTGTGTCTCGCTGTTGATGGGAGAGCAGTT |
| | TCTCGTCAGCTGGTGTTAGCCGGGCGGAGTGTGTTAGTCTCTCCAGAGGGAGGCTGGTTCCCCAGGGAAGCAGAGCCTGTGTGCGGGCAGCAGCTGTGTGCGGGCCTGGGGGTTGTTAAGTGCAATTATTTTTAATAAAAGGGGC |
| | ATTTGGAAAAAAAAAAAAAAGGTAGCAGTCGACAGATGAATTCTGCAGATCTGTGGCTTCTAGCTGCCCGGGTGGCATCCCTGTGACCCCTCCCCAGTGCCTCTCCTGGCCCTGGAAGTTGCCACTCCAGTGCCCACCAGCCTTG |
| | TCCTAATAAAATTAAGTTGCATCATTTTGTCTGACTAGGTGTCCTTCTATAATATTATGGGGTGGAGGGGGGTGGTATGGAGCAAGGGGCAAGTTGGGAAGACAACCTGAGTTGTTGGGATTCCAGGCATCGAGTAGATAAGTAG |
| | CATGGCGGGTTAATCATTAACTACACACGTGCGGACCGAGCGGCCTAAGCTCGCGAAGGAACCCCTAGTGATGGAGTTGGCCACTCCCTCTCTGCGCGCTCGCTCGCTCACTGAGGCCGGGCGACCAAAGGTCGCCCGACGCCCG |
| | GGCTTTGCCCGGGCGGCCTCAGTGAGCGAGCGAGCGCGCAGAGAGGGAGTGGCCAAGACGATTTAAATGACAAGCTTGGCGTAATCATGGTCATAGCTGTTTCCTGTGTGAAATTGTTATCCGCTCACAATTCCACACAACATAC |
| | GAGCCGGAAGCATAAAGTGTAAAGCCTGGGGTGCCTAATGAGTGAGCTAACTCACATTAATTGCGTTGCGCTCACTGCCCGCTTTCCAGTCGGGAAACCTGTCGTGCCAGCTGCATTAATGAATCGGCCAACGCGCGGGGAGAGG |
| | CGGTTTGCGTATTGGGCGCTCTTCCGCTTCCTCGCTCACTGACTCGCTGCGCTCGGTCGTTCGGCTGCGGCGAGCGGTATCAGCTCACTCAAAGGCGGTAATACGGTTATCCACAGAATCAGGGGATAACGCAGGAAAGAACATG |
| | TGAGCAAAAGGCCAGCAAAAGGCCAGGAACCGTAAAAAGGCCGCGTTGCTGGCGTTTTTCCATAGGCTCCGCCCCCCTGACGAGCATCACAAAAATCGACGCTCAAGTCAGAGGTGGCGAAACCCGACAGGACTATAAAGATACC |
| | AGGCGTTTCCCCCTGGAAGCTCCCTCGTGCGCTCTCCTGTTCCGACCCTGCCGCTTACCGGATACCTGTCCGCCTTTCTCCCTTCGGGAAGCGTGGCGCTTTCTCATAGCTCACGCTGTAGGTATCTCAGTTCGGTGTAGGTCGT |
| | TCGCTCCAAGCTGGGCTGTGTGCACGAACCCCCCGTTCAGCCCGACCGCTGCGCCTTATCCGGTAACTATCGTCTTGAGTCCAACCCGGTAAGACACGACTTATCGCCACTGGCAGCAGCCACTGGTAACAGGATTAGCAGAGCG |
| | AGGTATGTAGGCGGTGCTACAGAGTTCTTGAAGTGGTGGCCTAACTACGGCTACACTAGAAGAACAGTATTTGGTATCTGCGCTCTGCTGAAGCCAGTTACCTTCGGAAAAAGAGTTGGTAGCTCTTGATCCGGCAAACAAACCA |
| | CCGCTGGTAGCGGTGGTTTTTTTGTTTGCAAGCAGCAGATTACGCGCAGAAAAAAAGGATCTCAAGAAGATCCTTTGATCTTTTCTACGGGGTCTGACGCTCAGTGGAACGAAAACTCACGTTAAGGGATTTTGGTCATGAGATT |
| | ATCAAAAAGGATCTTCACCTAGATCCTTTTAAATTAAAAATGAAGTTTTAAATCAATCTAAAGTATATATGAGTAAACTTGGTCTGACAGTTAGAAAAACTCATCGAGCATCAAATGAAACTGCAATTTATTCATATCAGGATTA |
| | TCAATACCATATTTTTGAAAAAGCCGTTTCTGTAATGAAGGAGAAAACTCACCGAGGCAGTTCCATAGGATGGCAAGATCCTGGTATCGGTCTGCGATTCCGACTCGTCCAACATCAATACAACCTATTAATTTCCCCTCGTCAA |
| | AAATAAGGTTATCAAGTGAGAAATCACCATGAGTGACGACTGAATCCGGTGAGAATGGCAAAAGTTTATGCATTTCTTTCCAGACTTGTTCAACAGGCCAGCCATTACGCTCGTCATCAAAATCACTCGCATCAACCAAACCGTT |
| | ATTCATTCGTGATTGCGCCTGAGCGAGACGAAATACGCGATCGCTGTTAAAAGGACAATTACAAACAGGAATCGAATGCAACCGGCGCAGGAACACTGCCAGCGCATCAACAATATTTTCACCTGAATCAGGATATTCTTCTAAT |
| | ACCTGGAATGCTGTTTTTCCGGGGATCGCAGTGGTGAGTAACCATGCATCATCAGGAGTACGGATAAAATGCTTGATGGTCGGAAGAGGCATAAATTCCGTCAGCCAGTTTAGTCTGACCATCTCATCTGTAACATCATTGGCAA |
| | CGCTACCTTTGCCATGTTTCAGAAACAACTCTGGCGCATCGGGCTTCCCATACAAGCGATAGATTGTCGCACCTGATTGCCCGACATTATCGCGAGCCCATTTATACCCATATAAATCAGCATCCATGTTGGAATTTAATCGCGG |
| | CCTCGACGTTTCCCGTTGAATATGGCTCATACTCTTCCTTTTTCAATATTATTGAAGCATTTATCAGGGTTATTGTCTCATGAGCGGATACATATTTGAATGTATTTAGAAAAATAAACAAATAGGGGTTCCGCGCACATTTCCC |
| | CGAAAAGTGCCACCTGACGTCTAAGAAACCATTATTATCATGACATTAACCTATAAAAATAGGCGTATCACGAGGCCCTTTCGTCTCGCGCGTTTCGGTGATGACGGTGAAAACCTCTGACACATGCAGCTCCCGGAGACGGTCA |
| | CAGCTTGTCTGTAAGCGGATGCCGGGAGCAGACAAGCCCGTCAGGGCGCGTCAGCGGGTGTTGGCGGGTGTCGGGGCTGGCTTAACTATGCGGCATCAGAGCAGATTGTACTGAGAGTGCACCATATGCGGTGTGAAATACCGCA |
| | CAGATGCGTAAGGAGAAAATACCGCATCAGGC |
| 158 | >LVR422 Stuffer |
| | GCCATTCGCCATTCAGGCTGCGCAACTGTTGGGAAGGGCGATCGGTGCGGGCCTCTTCGCTATTACGCCAGCTGGCGAAAGGGGGATGTGCTGCAAGGCGATTAAGTTGGGTAACGCCAGGGTTTTCCCAGTCACGACGTTGTAA |
| | AACGACGGCCAGTGAATTCCTCGAGATTTAAATGACGTTGGCCACTCCCTCTCTGCGCGCTCGCTCGCTCACTGAGGCCGGGCGACCAAAGGTCGCCCGACGCCCGGGCTTTGCCCGGGCGGCCTCAGTGAGCGAGCGAGCGCGC |
| | AGAGAGGGAGTGGCCAACTCCATCACTAGGGGTTCCTGAGTTTAAACTTCGTCGAGGCCGCACGCGTAGGTTAATTTTTAAAAAGCAGTCAAAAGTCCAAGTGGCCCTTGGCAGCATTTACTCTCTCTGTTTGCTCTGGTTAATA |
| | ATCTCAGGAGCACAAACATTCCCCCTGTTCAAACATGTCCTAATACTCTGTCTCTGCAAGGGTCATCAGTAGTTTTCCATCTTACTCAACATCCTCCCAGTGAGTCATATGTTTGCTCACTGAAGGTTACTAGTTAACAGGCATC |
| | CCTTAAACAGGATATAAAAGGACTTCAGCAGGACTGCTCGAAACATCCCACTTCAGATCGCCTGGAGACGCCATCCACGCTGTTTTGACCTCCATAGAAGACACCGGGACCGATCCAGCCTCCGCGGCCGGGAACGGTGCATTGG |
| | AACGCGGATTCCCCGTGCCAAGAGTGACGTAAGTACCGCCTATAGACTCTATAGGCACACCCCTTTGGCTCTTATGCATGAACGGTGGAGGGCAGTGTAGTCTGAGCAGTACTCGTTGCTGCCGCGCGCGCCACCAGACATAATA |
| | GCTGACAGACTAACAGACTGTTCCTTTCCATGGGTCTTTTCTGCAGGCCACCATGGGAGTGAGGCACCCGCCCTGCTCCCACCGGCTCCTGGCCGTCTGCGCCCTCGTGTCCTTGGCAACCGCTGCACTCCTGGGGCACATCCTA |
| | CTCCATGATTTCCTGCTGGTTCCCCGAGAGCTGAGTGGCTCCTCCCCAGTCCTGGAGGAGACTCACCCAGCTCACCAGCAGGGAGCCAGCAGACCAGGGCCCCGGGATGCCCAGGCACACCCCGGCCGTCCCAGAGCAGTGCCCA |
| | CACAGTGCGACGTCCCCCCCAACAGCCGCTTCGATTGCGCCCCTGACAAGGCCATCACCCAGGAACAGTGCGAGGCCCGCGGCTGCTGCTACATCCCTGCAAAGCAGGGGCTGCAGGGAGCCCAGATGGGGCAGCCCTGGTGCTT |
| | CTTCCCACCCAGCTACCCCAGCTACAAGCTGGAGAACCTGAGCTCCTCTGAAATGGGCTACACGGCCACCCTGACCCGTACCACCCCCACCTTCTTCCCCAAGGACATCCTGACCCTGCGGCTGGACGTGATGATGGAGACTGAG |
| | AACCGCCTCCACTTCACGATCAAAGATCCAGCTAACAGGCGCTACGAGGTGCCCTTGGAGACCCCGCGTGTCCACAGCCGGGCACCGTCCCCACTCTACAGCGTGGAGTTCTCCGAGGAGCCCTTCGGGGTGATCGTGCACCGGC |
| | AGCTGGACGGCCGCGTGCTGCTGAACACGACGGTGGCGCCCCTGTTCTTTGCGGACCAGTTCCTTCAGCTGTCCACCTCGCTGCCCTCGCAGTATATCACAGGCCTCGCCGAGCACCTCAGTCCCCTGATGCTCAGCACCAGCTG |
| | GACCAGGATCACCCTGTGGAACCGGGACCTTGCGCCCACGCCCGGTGCGAACCTCTACGGGTCTCACCCTTTCTACCTGGCGCTGGAGGACGGCGGGTCGGCACACGGGGTGTTCCTGCTAAACAGCAATGCCATGGATGTGGTC |
| | CTGCAGCCGAGCCCTGCCCTTAGCTGGAGGTCGACAGGTGGGATCCTGGATGTCTACATCTTCCTGGGCCCAGAGCCCAAGAGCGTGGTGCAGCAGTACCTGGACGTTGTGGGATACCCGTTCATGCCGCCATACTGGGGCCTGG |
| | GCTTCCACCTGTGCCGCTGGGGCTACTCCTCCACCGCTATCACCCGCCAGGTGGTGGAGAACATGACCAGGGCCCACTTCCCCCTGGACGTCCAATGGAACGACCTGGACTACATGGACTCCCGGAGGGACTTCACGTTCAACAA |
| | GGATGGCTTCCGGGACTTCCCGGCCATGGTGCAGGAGCTGCACCAGGGCGGCCGGCGCTACATGATGATCGTGGATCCTGCCATCAGCAGCTCGGGCCCTGCCGGGAGCTACAGGCCCTACGACGAGGGTCTGCGGAGGGGGGTT |
| | TTCATCACCAACGAGACCGGCCAGCCGCTGATTGGGAAGGTATGGCCCGGGTCCACTGCCTTCCCCGACTTCACCAACCCCACAGCCCTGGCCTGGTGGGAGGACATGGTGGCTGAGTTCCATGACCAGGTGCCCTTCGACGGCA |
| | TGTGGATTGACATGAACGAGCCTTCCAACTTCATCAGAGGCTCTGAGGACGGCTGCCCCAACAATGAGCTGGAGAACCCACCCTACGTGCCTGGGGTGGTTGGGGGGACCCTCCAGGCGGCCACCATCTGTGCCTCCAGCCACCA |
| | GTTTCTCTCCACACACTACAACCTGCACAACCTCTACGGCCTGACCGAAGCCATCGCCTCCCACAGGGCGCTGGTGAAGGCTCGGGGGACACGCCCATTTGTGATCTCCCGCTCGACCTTTGCTGGCCACGGCCGATACGCCGGC |
| | CACTGGACGGGGGACGTGTGGAGCTCCTGGGAGCAGCTCGCCTCCTCCGTGCCAGAAATCCTGCAGTTTAACCTGCTGGGGGTGCCTCTGGTCGGGGCCGACGTCTGCGGCTTCCTGGGCAACACCTCAGAGGAGCTGTGTGTGC |
| | GCTGGACCCAGCTGGGGGCCTTCTACCCCTTCATGCGGAACCACAACAGCCTGCTCAGTCTGCCCCAGGAGCCGTACAGCTTCAGCGAGCCGGCCCAGCAGGCCATGAGGAAGGCCCTCACCCTGCGCTACGCACTCCTCCCCCA |
| | CCTCTACACACTGTTCCACCAGGCCCACGTCGCGGGGGAGACCGTGGCCCGGCCCCTCTTCCTGGAGTTCCCCAAGGACTCTAGCACCTGGACTGTGGACCACCAGCTCCTGTGGGGGGAGGCCCTGCTCATCACCCCAGTGCTC |
| | CAGGCCGGGAAGGCCGAAGTGACTGGCTACTTCCCCTTGGGCACATGGTACGACCTGCAGACGGTGCCAATAGAGGCCCTTGGCAGCCTCCCACCCCCACCTGCAGCTCCCCGTGAGCCAGCCATCCACAGCGAGGGGCAGTGGG |
| | TGACGCTGCCGGCCCCCCTGGACACCATCAACGTCCACCTCCGGGCTGGGTACATCATCCCCCTGCAGGGCCCTGGCCTCACAACCACAGAGTCCCGCCAGCAGCCCATGGCCCTGGCTGTGGCCCTGACCAAGGGTGGAGAGGC |
| | CCGAGGGGAGCTGTTCTGGGACGATGGAGAGAGCCTGGAAGTGCTGGAGCGAGGGGCCTACACACAGGTCATCTTCCTGGCCAGGAATAACACGATCGTGAATGAGCTGGTACGTGTGACCAGTGAGGGAGCTGGCCTGCAGCTG |
| | CAGAAGGTGACTGTCCTGGGCGTGGCCACGGCGCCCCAGCAGGTCCTCTCCAACGGTGTCCCTGTCTCCAACTTCACCTACAGCCCCGACACCAAGGTCCTGGACATCTGTGTCTCGCTGTTGATGGGAGAGCAGTTTCTCGTCA |
| | GCTGGTGTTAGCGAGCGGCCGCTCTTAGTAGCAGTATCGATCCCAGCCCACTTTTCCCCAATACGACTACGAGATCTGTGGCTTCTAGCTGCCCGGGTGGCATCCCTGTGACCCCTCCCCAGTGCCTCTCCTGGCCCTGGAAGTT |
| | GCCACTCCAGTGCCCACCAGCCTTGTCCTAATAAAATTAAGTTGCATCATTTTGTCTGACTAGGTGTCCTTCTATAATATTATGGGGTGGAGGGGGGTGGTATGGAGCAAGGGGCAAGTTGGGGTCCTTCAACGTCGAACTGTAG |
| | CACTCTGGATAGCTAGCTAGCGTTTGTAGATAGTTTCAAGGATAGCAACCAACAACACGATAGTTTAGAAATTGTTCTAGATCTTCTGCTACTACTCGAGAGATAGTTGACTTCTAGAAGACTCTGGATAGAGAGCTTCCTGTGG |
| | TTTCTATTTCAACACTTCGAGCGATTTAGATAGAGGCGTCAATTGAGAACAAAAACTGGACACCTGTTTCTAGAACACTAGTGCTAGTCTTCTCAAGGCTTGATACGTCCTGCCACTCACACACAAAAAACCAACACACAGATTA |
| | ATGAAAATAAAGATCTTTTATTAGAGAGCGAGTGGATAGTTTAGAGTCGCTTGAGACTTATCCTGTGTCCAACTAGCCAAGAGTAGAGAGAGAGTGACTAGGTGGATCGAGAGATCGTCGTCTATTTGACTGCTAGCCTGTGTTC |
| | TCGTCGTAGTCCAAATCTTCAATACTCTGGCGACTAGTAACTTGAATCTGTGTATCTAACACGACAAGGCTACTCCTTTGAGTTTCTAGTGATAAGTGCGACTGGATCTAGCTAGTTGAGTTTCTAGAAACTACGTCCAAAGCTA |
| | CTAGCACTACACGTGCGGACCGAGCGGCCTAAGCTCGCGAAGGAACCCCTAGTGATGGAGTTGGCCACTCCCTCTCTGCGCGCTCGCTCGCTCACTGAGGCCGGGCGACCAAAGGTCGCCCGACGCCCGGGCTTTGCCCGGGCGG |
| | CCTCAGTGAGCGAGCGAGCGCGCAGAGAGGGAGTGGCCAAGACGATTTAAATGACAAGCTTGGCGTAATCATGGTCATAGCTGTTTCCTGTGTGAAATTGTTATCCGCTCACAATTCCACACAACATACGAGCCGGAAGCATAAA |
| | GTGTAAAGCCTGGGGTGCCTAATGAGTGAGCTAACTCACATTAATTGCGTTGCGCTCACTGCCCGCTTTCCAGTCGGGAAACCTGTCGTGCCAGCTGCATTAATGAATCGGCCAACGCGCGGGGAGAGGCGGTTTGCGTATTGGG |
| | CGCTCTTCCGCTTCCTCGCTCACTGACTCGCTGCGCTCGGTCGTTCGGCTGCGGCGAGCGGTATCAGCTCACTCAAAGGCGGTAATACGGTTATCCACAGAATCAGGGGATAACGCAGGAAAGAACATGTGAGCAAAAGGCCAGC |
| | AAAAGGCCAGGAACCGTAAAAAGGCCGCGTTGCTGGCGTTTTTCCATAGGCTCCGCCCCCCTGACGAGCATCACAAAAATCGACGCTCAAGTCAGAGGTGGCGAAACCCGACAGGACTATAAAGATACCAGGCGTTTCCCCCTGG |
| | AAGCTCCCTCGTGCGCTCTCCTGTTCCGACCCTGCCGCTTACCGGATACCTGTCCGCCTTTCTCCCTTCGGGAAGCGTGGCGCTTTCTCATAGCTCACGCTGTAGGTATCTCAGTTCGGTGTAGGTCGTTCGCTCCAAGCTGGGC |
| | TGTGTGCACGAACCCCCCGTTCAGCCCGACCGCTGCGCCTTATCCGGTAACTATCGTCTTGAGTCCAACCCGGTAAGACACGACTTATCGCCACTGGCAGCAGCCACTGGTAACAGGATTAGCAGAGCGAGGTATGTAGGCGGTG |
| | CTACAGAGTTCTTGAAGTGGTGGCCTAACTACGGCTACACTAGAAGAACAGTATTTGGTATCTGCGCTCTGCTGAAGCCAGTTACCTTCGGAAAAAGAGTTGGTAGCTCTTGATCCGGCAAACAAACCACCGCTGGTAGCGGTGG |
| | TTTTTTTGTTTGCAAGCAGCAGATTACGCGCAGAAAAAAAGGATCTCAAGAAGATCCTTTGATCTTTTCTACGGGGTCTGACGCTCAGTGGAACGAAAACTCACGTTAAGGGATTTTGGTCATGAGATTATCAAAAAGGATCTTC |
| | ACCTAGATCCTTTTAAATTAAAAATGAAGTTTTAAATCAATCTAAAGTATATATGAGTAAACTTGGTCTGACAGTTAGAAAAACTCATCGAGCATCAAATGAAACTGCAATTTATTCATATCAGGATTATCAATACCATATTTTT |
| | GAAAAAGCCGTTTCTGTAATGAAGGAGAAAACTCACCGAGGCAGTTCCATAGGATGGCAAGATCCTGGTATCGGTCTGCGATTCCGACTCGTCCAACATCAATACAACCTATTAATTTCCCCTCGTCAAAAATAAGGTTATCAAG |
| | TGAGAAATCACCATGAGTGACGACTGAATCCGGTGAGAATGGCAAAAGTTTATGCATTTCTTTCCAGACTTGTTCAACAGGCCAGCCATTACGCTCGTCATCAAAATCACTCGCATCAACCAAACCGTTATTCATTCGTGATTGC |
| | GCCTGAGCGAGACGAAATACGCGATCGCTGTTAAAAGGACAATTACAAACAGGAATCGAATGCAACCGGCGCAGGAACACTGCCAGCGCATCAACAATATTTTCACCTGAATCAGGATATTCTTCTAATACCTGGAATGCTGTTT |
| | TTCCGGGGATCGCAGTGGTGAGTAACCATGCATCATCAGGAGTACGGATAAAATGCTTGATGGTCGGAAGAGGCATAAATTCCGTCAGCCAGTTTAGTCTGACCATCTCATCTGTAACATCATTGGCAACGCTACCTTTGCCATG |
| | TTTCAGAAACAACTCTGGCGCATCGGGCTTCCCATACAAGCGATAGATTGTCGCACCTGATTGCCCGACATTATCGCGAGCCCATTTATACCCATATAAATCAGCATCCATGTTGGAATTTAATCGCGGCCTCGACGTTTCCCGT |
| | TGAATATGGCTCATACTCTTCCTTTTTCAATATTATTGAAGCATTTATCAGGGTTATTGTCTCATGAGCGGATACATATTTGAATGTATTTAGAAAAATAAACAAATAGGGGTTCCGCGCACATTTCCCCGAAAAGTGCCACCTG |
| | ACGTCTAAGAAACCATTATTATCATGACATTAACCTATAAAAATAGGCGTATCACGAGGCCCTTTCGTCTCGCGCGTTTCGGTGATGACGGTGAAAACCTCTGACACATGCAGCTCCCGGAGACGGTCACAGCTTGTCTGTAAGC |
| | GGATGCCGGGAGCAGACAAGCCCGTCAGGGCGCGTCAGCGGGTGTTGGCGGGTGTCGGGGCTGGCTTAACTATGCGGCATCAGAGCAGATTGTACTGAGAGTGCACCATATGCGGTGTGAAATACCGCACAGATGCGTAAGGAGA |
| | AAATACCGCATCAGGC |
| | >LVRSP0412 insertion |
| | GGTACCGAGCTCGGATCCACTAGTAACGGCCGCCAGTGTGCTGGAAT |
| 106 | >HLP |
| | GTTTGCTGCTTGCAATGTTTGCCCATTTTAGGGTGGACACAGGACGCTGTGGTTTCTGAGCCAGGGGGCGACTCAGATCCCAGCCAGTGGACTTAGCCCCTGTTTGCTCCTCCGATAACTGGGGTGACCTTGGTTAATATTCACC |
| | AGCAGCCTCCCCCGTTGCCCCTCTGGATCCACTGCTTAAATACGGACGAGGACAGGGCCCTGTCTCCTCAGCTTCAGGCACCACCACTGACCTGGGACAGTGAATC |
| 107 | GGGGGCGACTCAGATCCCAGCC |
| 108 | AGGACGCTGTGGTTTCTGAG |
| 109 | AGGTCACCCCAGTTATCGGA |
| 110 | |
| 111 | |
| 112 | |
| 113 | |
| 270 | >CRM_SP0107 |
| | CCCTGGAGAGTCCTTTAGCAGGGCAAAGTGCAACATAGGCAGACCTTAAGGGATGACTCAGTAACAGATAAGCTTTGTGTGCCTGCAGGGGGATGGGAAGAGGGTGGGGCAGGAGAGGGACATAAAAGGGCTCTGAGGCATTGTA |
| | CTGTGAATTCCTTCAGTCTCCTGTTTGGAGAAGACAGAGCCAATGAGGCCCTCGTTCCAGGGAAACAGAATATGCTCAGCATGACGCAGCACTCCCTGAACTTTCCGGTTACATCACCCAATAGCTGAGATCAGA |
| 271 | >CRM_SP0109 |
| | CCCTGGAGAGTCCTTTAGCAGGGCAAAGTGCAACATAGGCAGACCTTAAGGGATGACTCAGTAACAGATAAGCTTTGTGTGCCTGCAGGGGGATGGGAAGAGGGTGGGGCAGGAGAGGGACATAAAAGGGCTCTGAGGCATTGTA |
| | CTGTGAATTCCTTCAGTCTCCTGCTCTGCTCAGCCAGTCAGCCCTGCCTCCCTTGTTTAGGACCACACAGCACTGCTGGGTGTCTGCCTTTCCTTG |
| 273 | >CRM_SP0111 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTTTGGAGAAGACAGAGCCAATGAGGCCCTCGTTCCAGGGAAACAG |
| | AATATGCTCAGCATGACGCAGCACTCCCTGAACTTTCCGGTTACATCACCCAATAGCTGAGATCAGA |
| 274 | >CRM_SP0112 |
| | CACTGAACCCTTGACCCCTGCCCTGCAGCCCCCGCAGCTTGCTGTTTGCCCACTCTATTTGCCCAGCCCCAGAGGTTAATTTTTAAAAAGCAGTCAAAAGTCCAAGTGGCCCTTGGCAGCATTTACTCTCTCTGTTTGCTCTGGT |
| | TAATAATCTCAGGAGCACAAACATTCCTTTGGAGAAGACAGAGCCAATGAGGCCCTCGTTCCAGGGAAACAGAATATGCTCAGCATGACGCAGCACTCCCTGAACTTTCCGGTTACATCACCCAATAGCTGAGATCAGA |
| 275 | >CRM_SP0113 |
| | CACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAACATAGGCAGACCTTAAGGGATGACTCAGTAACAGATAAGC |
| | TTTGTGTGCCTGCAGGGGGATGGGAAGAGGGTGGGGCAGGAGAGGGACATAAAAGGGCTCTGAGGCATTGTACTGTGAATTCCTTCAGTCTCCTG |
| 276 | >CRM_SP0115 |
| | CCCTGGAGAGTCCTTTAGCAGGGCAAAGTGCAACATAGGCAGACCTTAAGGGATGACTCAGTAACAGATAAGCTTTGTGTGCCTGCAGGGGGATGGGAAGAGGGTGGGGCAGGAGAGGGACATAAAAGGGCTCTGAGGCATTGTA |
| | CTGTGAATTCCTTCAGTCTCCTGCTCTGCTCAGCCAGTCAGCCCTGCCTCCCTTGTTTAGGACCACACAGCACTGCTGGGTGTCTGCCTTTCCTTG |
| 277 | >CRM_SP0116 |
| | CCCTGGAGAGTCCTTTAGCAGGGCAAAGTGCAACATAGGCAGACCTTAAGGGATGACTCAGTAACAGATAAGCTTTGTGTGCCTGCAGGGGGATGGGAAGAGGGTGGGGCAGGAGAGGGACATAAAAGGGCTCTGAGGCATTGTA |
| | CTGTGAATTCCTTCAGTCTCCTG |
| 278 | >CRM_SP0121 |
| | CCCTGGAGAGTCCTTTAGCAGGGCAAAGTGCAACATAGGCAGACCTTAAGGGATGACTCAGTAACAGATAAGCTTTGTGTGCCTGCACCCTGGAGAGTCCTTTAGCAGGGCAAAGTGCAACATAGGCAGACCTTAAGGGATGACT |
| | CAGTAACAGATAAGCTTTGTGTGCCTGCA |
| 279 | >CRM_SP0124 |
| | CACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAACATAGGCAGACCTTAAGGGATGACTCAGTAACAGATAAGC |
| | TTTGTGTGCCTGCA |
| 280 | >CRM_SP0127 (CRM_LVR 127) |
| | AGAATGAACATTGAACTTTGGACTATACCTGAGGGGTGAGGTAAACAACAGGACTATAAATGGCCCGGGAGGCGCCCTTTGGACCTTTTGCAATCCTGGCGCACTGAACCCTTGACCCCTGCCCTGCAGCCCCCGCAGCTTGCTG |
| | TTTGCCCACTCTATTTGCCCAGCCCCAGGGACAGAGCTGATCCTTGAACTCTTAAGTTCCAC |
| 281 | >CRM_SP0127A1 (CRM_LVR_127_A1) |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTGTACCAGAATGAACATTGAACTTTGGACTATACCTGAGGGGTGA |
| | GGTAAACAACAGGACTATAAATGGCCCGGGAGGCGCCCTTTGGACCTTTTGCAATCCTGGCGCACTGAACCCTTGACCCCTGCCCTGCAGCCCCCGCAGCTTGCTGTTTGCCCACTCTATTTGCCCAGCCCCAGGGACAGAGCTG |
| | ATCCTTGAACTCTTAAGTTCCAC |
| 282 | >CRM_SP0127V1 (CRM_LVR 127 V1) |
| | AAGCAAATATTTGTGGTTATGGATTAACTCGAACTGTTTGCCCACTCTATTTGCCCTGTACCAGAATGAACATTGAACTTTGGACTATACCTGAGGGGTGAGGTAAACAACAGGACTATAAATGGCCCGGGAGGCGCCCTTTGGA |
| | CCTTTTGCAATCCTGGCGCACTGAACCCTTGACCCCTGCCCTGCAGCCCCCGCAGCTTGCTGTTTGCCCACTCTATTTGCCCAGCCCCAGGGACAGAGCTGATCCTTGAACTCTTAAGTTCCAC |
| 283 | >CRM_SP0127V2 (CRM_LVR_127_V2) |
| | GGCGCCCTTTGGACCTTTTGCAATCCTGGAGCAAACAGCAAACACTGTACCAGAATGAACATTGAACTTTGGACTATACCTGAGGGGTGAGGTAAACAACAGGACTATAAATGGCCCGGGAGGCGCCCTTTGGACCTTTTGCAAT |
| | CCTGGCGCACTGAACCCTTGACCCCTGCCCTGCAGCCCCCGCAGCTTGCTGTTTGCCCACTCTATTTGCCCAGCCCCAGGGACAGAGCTGATCCTTGAACTCTTAAGTTCCAC |
| 284 | >CRM_SP0128 |
| | AGAATGAACATTGAACTTTGGACTATACCTGAGGGGTGAGGTAAACAACAGGACTATAAATAGAATGAACATTGAACTTTGGACTATACCTGAGGGGTGAGGTAAACAACAGGACTATAAATCACTGAACCCTTGACCCCTGCCC |
| | TGCAGCCCCCGCAGCTTGCTGTTTGCCCACTCTATTTGCCCAGCCCCAG |
| 285 | >CRM_SP0131 (CRM_LVR_131) |
| | GGCCCGGGAGGCGCCCTTTGGACCTTTTGCAATCCTGGCGCACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAA |
| | CATAGGCAGACCTTAAGGGATGACTCAGTAACAGATAAGCTTTGTGTGCCTGCA |
| 286 | >CRM_SP0132 (CRM_LVR 132 |
| | AGAATGAACATTGAACTTTGGACTATACCTGAGGGGTGAGGTAAACAACAGGACTATAAATGGCCCGGGAGGCGCCCTTTGGACCTTTTGCAATCCTGGCGCACTGAACCCTTGACCCCTGCCCTGCAGCCCCCGCAGCTTGCTG |
| | TTTGCCCACTCTATTTGCCCAGCCCCAGCCCTGGAGAGTCCTTTAGCAGGGCAAAGTGCAACATAGGCAGACCTTAAGGGATGACTCAGTAACAGATAAGCTTTGTGTGCCTGCA |
| 287 | >CRM_SP0133 (CRM_LVR_133) |
| | AGAATGAACATTGAACTTTGGACTATACCTGAGGGGTGAGGTAAACAACAGGACTATAAATGGCCCGGGAGGCGCCCTTTGGACCTTTTGCAATCCTGGCG |
| 288 | >CRM_SP0155 |
| | GCTGGTTTCTTATAAAACTGATGGAAGATACAAACACTATTAAAGAACTGTTTGCATGTTGCAAATGATGTCCAAAGTCCAAACATTGTTAATAATTAATACTCCAATAAACATCATGTCAGAATTTCTGTTTTCTTTTCCCTTT |
| | GAACCTTTGCAGGATTGCCACATCATCAGGACCACACCTTCATCAGGAATGAATAT |
| 289 | >CRM_SP0158 |
| | ATTGGCATCTTCTATTGCTTTTCCTGGTGACTTCATTTTTCACTCTTGGCTAAAAATGGGTCTCTGATGATTTATTCTATCCTGGGTGTTGACAAGCTGAAGAAGTTGTGTGGGGCCTGCTGCCAGTAACCCTGGGTGACGAAGC |
| | GTGACTCACCACTCCGAGGTCAGTGGGGGGATGGAAGGCAGGGGAGTCAGCTGACAAGATCTGCTGCTTTGTCACCAGGCCTTCTGC |
| 290 | >CRM_SP0163 |
| | TGAAATGCCTGCCATATATTAGTGCCCTGAAGTCCAAAGGTAGAGGAACCGAGTGTTTAAAAATTACTGTGGCTGTGGAGTCAACATGATGTAAAAAAACAAACATTTGGATAACACCAAGAAGCCAGATATGGTTGAAATGTTG |
| | ACTGGTTGACAAAAATAATTTGGGTTGCTTAATGGTGCACAAAGGTAATGCAAAA |
| 291 | >CRM_SP0236 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTG |
| 292 | >CRM_SP0239 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTGAGGTTAATTTTTAAAAAGCAGTCAAAAGTCCAAGTGGCCCTT |
| | GGCAGCATTTACTCTCTCTGTTTGCTCTGGTTAATAATCTCAGGAGCACAAACATTCCGGCCCGGGAGGCGCCCTTTGGACCTTTTGCAATCCTGGCGCACTGAACCCTTGACCCCTGCCCTGCAGCCCCCGCAGCTTGCTGTTT |
| | GCCCACTCTATTTGCCCAGCCCCAGCCCTGGAGAGTCCTTTAGCAGGGCAAAGTGCAACATAGGCAGACCTTAAGGGATGACTCAGTAACAGATAAGCTTTGTGTGCCTGCA |
| 293 | >CRM_SP0240 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTG |
| 294 | >CRM_SP0241 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGCACTG |
| | AACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAACATAGGCAGACCTTAAGGGATGACTCAGTAACAGATAAGCTTTGT |
| | GTGCCTGCA |
| 295 | >CRM_SP0242 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGCACTG |
| | AACCCTTGACCCCTGCCCTGCAGCCCCCGCAGCTTGCTGTTTGCCCACTCTATTTGCCCAGCCCCAG |
| 296 | >CRM_SP0243 |
| | TGAAATGCCTGCCATATATTAGTGCCCTGAAGTCCAAAGGTAGAGGAACCGAGTGTTTAAAAATTACTGTGGCTGTGGAGTCAACATGATGTAAAAAAACAAACATTTGGATAACACCAAGAAGCCAGATATGGTTGAAATGTTG |
| | ACTGGTTGACAAAAATAATTTGGGTTGCTTAATGGTGCACAAAGGTAATGCAAAAAGGTTAATTTTTAAAAAGCAGTCAAAAGTCCAAGTGGCCCTTGGCAGCATTTACTCTCTCTGTTTGCTCTGGTTAATAATCTCAGGAGCA |
| | CAAACATTCCGGCCCGGGAGGCGCCCTTTGGACCTTTTGCAATCCTGGCGCACTGAACCCTTGACCCCTGCCCTGCAGCCCCCGCAGCTTGCTGTTTGCCCACTCTATTTGCCCAGCCCCAGCCCTGGAGAGTCCTTTAGCAGGG |
| | CAAAGTGCAACATAGGCAGACCTTAAGGGATGACTCAGTAACAGATAAGCTTTGTGTGCCTGCA |
| 297 | >CRM_SP0244 |
| | CTTTGGACCTTTTGCAATCCTGGCGCACTGAACCCTTGACCCCTGCCCTGCAGCCCCCGCAGCTTGCTGTTTGCCCACTCTATTTGCCCAGCCCCAGTGAAATGCCTGCCATATATTAGTGCCCTGAAGTCCAAAGGTAGAGGAA |
| | CCGAGTGTTTAAAAATTACTGTGGCTGTGGAGTCAACATGATGTAAAAAAACAAACATTTGGATAACACCAAGAAGCCAGATATGGTTGAAATGTTGACTGGTTGACAAAAATAATTTGGGTTGCTTAATGGTGCACAAAGGTAA |
| | TGCAAAA |
| 298 | >CRM_SP0246 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGCACTG |
| | AACCCTTGACCCCTGCCCTGCAGCCCCCGCAGCTTGCTGTTTGCCCACTCTATTTGCCCAGCCCCAG |
| 299 | >CRM_SP0247 |
| | GCTGGTTTCTTATAAAACTGATGGAAGATACAAACACTATTAAAGAACTGTTTGCATGTTGCAAATGATGTCCAAAGTCCAAACATTGTTAATAATTAATACTCCAATAAACATCATGTCAGAATTTCTGTTTTCTTTTCCCTTT |
| | GAACCTTTGCAGGATTGCCACATCATCAGGACCACACCTTCATCAGGAATGAATATCAGGCTTTCACTTTCTCGCCAACTTACAAGGCCTTTCTGTGTAAACAATACCTGAACCTTTACCCCGTTGCCCGGCAACGGCCAGGTCT |
| | GTGCCAAGTGTTTG |
| 300 | >CRM_SP0248 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGCACTG |
| | AACCCTTGACCCCTGCCCTGCAGCCCCCGCAGCTTGCTGTTTGCCCACTCTATTTGCCCAGCCCCAGCAGGCTTTCACTTTCTCGCCAACTTACAAGGCCTTTCTGTGTAAACAATACCTGAACCTTTACCCCGTTGCCCGGCAA |
| | CGGCCAGGTCTGTGCCAAGTGTTTG |
| 301 | >CRM_SP0249 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGCACTG |
| | AACCCTTGACCCCTGCCCTGCAGCCCCCGCAGCTTGCTGTTTGCCCACTCTATTTGCCCAGCCCCAGCAGGCTTTCACTTTCTCGCCAACTTACAAGGCCTTTCTGTGTAAACAATACCTGAACCTTTACCCCGTTGCCCGGCAA |
| | CGGCCAGGTCTGTGCCAAGTGTTTG |
| 302 | >CRM_SP0250 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGCACTG |
| | AACCCTTGACCCCTGCCCTGCAGCCCCCGCAGCTTGCTGTTTGCCCACTCTATTTGCCCAGCCCCAGGCTGGTTTCTTATAAAACTGATGGAAGATACAAACACTATTAAAGAACTGTTTGCATGTTGCAAATGATGTCCAAAGT |
| | CCAAACATTGTTAATAATTAATACTCCAATAAACATCATGTCAGAATTTCTGTTTTCTTTTCCCTTTGAACCTTTGCAGGATTGCCACATCATCAGGACCACACCTTCATCAGGAATGAATAT |
| 303 | >CRM_SP0251 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGCACTG |
| | AACCCTTGACCCCTGCCCTGCAGCCCCCGCAGCTTGCTGTTTGCCCACTCTATTTGCCCAGCCCCAGAAGCAAATATTTGTGGTTATGGATTAACTCGAACTGTTTGCCCACTCTATTTGCCC |
| 304 | >CRM_SP0252 |
| | AAGCAAATATTTGTGGTTATGGATTAACTCGAACTGTTTGCCCACTCTATTTGCCCCAGGCTTTCACTTTCTCGCCAACTTACAAGGCCTTTCTGTGTAAACAATACCTGAACCTTTACCCCGTTGCCCGGCAACGGCCAGGTCT |
| | GTGCCAAGTGTTTG |
| 305 | >CRM_SP0253 |
| | AAGCAAATATTTGTGGTTATGGATTAACTCGAACTGTTTGCCCACTCTATTTGCCCCAGGCTTTCACTTTCTCGCCAACTTACAAGGCCTTTCTGTGTAAACAATACCTGAACCTTTACCCCGTTGCCCGGCAACGGCCAGGTCT |
| | GTGCCAAGTGTTTG |
| 306 | >CRM_SP0254 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGCAGGC |
| | TTTCACTTTCTCGCCAACTTACAAGGCCTTTCTGTGTAAACAATACCTGAACCTTTACCCCGTTGCCCGGCAACGGCCAGGTCTGTGCCAAGTGTTTG |
| 307 | >CRM_SP0255 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGCAGGC |
| | TTTCACTTTCTCGCCAACTTACAAGGCCTTTCTGTGTAAACAATACCTGAACCTTTACCCCGTTGCCCGGCAACGGCCAGGTCTGTGCCAAGTGTTTG |
| 308 | >CRM_SP0256 |
| | ATTGGCATCTTCTATTGCTTTTCCTGGTGACTTCATTTTTCACTCTTGGCTAAAAATGGGTCTCTGATGATTTATTCTATCCTGGGTGTTGACAAGCTGAAGAAGTTGTGTGGGGCCTGCTGCCAGTAACCCTGGGTGACGAAGC |
| | GTGACTCACCACTCCGAGGTCAGTGGGGGGATGGAAGGCAGGGGAGTCAGCTGACAAGATCTGCTGCTTTGTCACCAGGCCTTCTGCCAGGCTTTCACTTTCTCGCCAACTTACAAGGCCTTTCTGTGTAAACAATACCTGAACC |
| | TTTACCCCGTTGCCCGGCAACGGCCAGGTCTGTGCCAAGTGTTTG |
| 309 | >CRM_SP0257 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTGGCTGGTTTCTTATAAAACTGATGGAAGATACAAACACTATTA |
| | AAGAACTGTTTGCATGTTGCAAATGATGTCCAAAGTCCAAACATTGTTAATAATTAATACTCCAATAAACATCATGTCAGAATTTCTGTTTTCTTTTCCCTTTGAACCTTTGCAGGATTGCCACATCATCAGGACCACACCTTCA |
| | TCAGGAATGAATAT |
| 310 | >CRM_SP0258 |
| | CTGTTTGCTGCTTGCAATGTTTGCCCATTTTAGGGAGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAG |
| | GCGCCCTTTGGACCTTTTGCAATCCTGGCGCACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAACATAGGCAGA |
| | CCTTAAGGGATGACTCAGTAACAGATAAGCTTTGTGTGCCTGCA |
| 311 | >CRM_SP0259 |
| | CTGTTTGCTGCTTGCAATGTTTGCCCATTTTAGGGGCTGGTTTCTTATAAAACTGATGGAAGATACAAACACTATTAAAGAACTGTTTGCATGTTGCAAATGATGTCCAAAGTCCAAACATTGTTAATAATTAATACTCCAATAA |
| | ACATCATGTCAGAATTTCTGTTTTCTTTTCCCTTTGAACCTTTGCAGGATTGCCACATCATCAGGACCACACCTTCATCAGGAATGAATAT |
| 312 | >CRM_SP0264 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTG |
| 313 | >CRM_SP0265 (CRM_LVR_131_A1) |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTGTACCGGCCCGGGAGGCGCCCTTTGGACCTTTTGCAATCCTGGC |
| | GCACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAACATAGGCAGACCTTAAGGGATGACTCAGTAACAGATAAG |
| | CTTTGTGTGCCTGCA |
| 314 | >CRM_SP0266 (CRM_LVR_131_V1) |
| | AAGCAAATATTTGTGGTTATGGATTAACTCGAACTGTTTGCCCACTCTATTTGCCCTGTACCGGCCCGGGAGGCGCCCTTTGGACCTTTTGCAATCCTGGCGCACTGAACCCTTGACCCCTGCCCTGCAGCCCCCGCAGCTTGCT |
| | GTTTGCCCACTCTATTTGCCCAGCCCCAGCCCTGGAGAGTCCTTTAGCAGGGCAAAGTGCAACATAGGCAGACCTTAAGGGATGACTCAGTAACAGATAAGCTTTGTGTGCCTGCA |
| 315 | >CRM_SP0267 (CRM_LVR_131_V2) |
| | GGCGCCCTTTGGACCTTTTGCAATCCTGGAGCAAACAGCAAACACTGTACCGGCCCGGGAGGCGCCCTTTGGACCTTTTGCAATCCTGGCGCACTGAACCCTTGACCCCTGCCCTGCAGCCCCCGCAGCTTGCTGTTTGCCCACT |
| | CTATTTGCCCAGCCCCAGCCCTGGAGAGTCCTTTAGCAGGGCAAAGTGCAACATAGGCAGACCTTAAGGGATGACTCAGTAACAGATAAGCTTTGTGTGCCTGCA |
| 316 | >CRM_SP0268 (CRM_LVR_132_A1) |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTGTACCAGAATGAACATTGAACTTTGGACTATACCTGAGGGGTGA |
| | GGTAAACAACAGGACTATAAATGGCCCGGGAGGCGCCCTTTGGACCTTTTGCAATCCTGGCGCACTGAACCCTTGACCCCTGCCCTGCAGCCCCCGCAGCTTGCTGTTTGCCCACTCTATTTGCCCAGCCCCAGCCCTGGAGAGT |
| | CCTTTAGCAGGGCAAAGTGCAACATAGGCAGACCTTAAGGGATGACTCAGTAACAGATAAGCTTTGTGTGCCTGCA |
| 317 | >CRM_SP0269 (CRM_LVR_132_V1) |
| | AAGCAAATATTTGTGGTTATGGATTAACTCGAACTGTTTGCCCACTCTATTTGCCCTGTACCAGAATGAACATTGAACTTTGGACTATACCTGAGGGGTGAGGTAAACAACAGGACTATAAATGGCCCGGGAGGCGCCCTTTGGA |
| | CCTTTTGCAATCCTGGCGCACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAACATAGGCAGACCTTAAGGGATG |
| | ACTCAGTAACAGATAAGCTTTGTGTGCCTGCA |
| 318 | >CRM_SP0270 (CRM_LVR_132_V2) |
| | GGCGCCCTTTGGACCTTTTGCAATCCTGGAGCAAACAGCAAACACTGTACCAGAATGAACATTGAACTTTGGACTATACCTGAGGGGTGAGGTAAACAACAGGACTATAAATGGCCCGGGAGGCGCCCTTTGGACCTTTTGCAAT |
| | CCTGGCGCACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAACATAGGCAGACCTTAAGGGATGACTCAGTAACA |
| | GATAAGCTTTGTGTGCCTGCA |
| 319 | >CRM_SP0271 (CRM_LVR 133_A1) |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTGTACCAGAATGAACATTGAACTTTGGACTATACCTGAGGGGTGA |
| | GGTAAACAACAGGACTATAAATGGCCCGGGAGGCGCCCTTTGGACCTTTTGCAATCCTGGCG |
| 320 | >CRM_SP0272 (CRM_LVR_133_V1) |
| | AAGCAAATATTTGTGGTTATGGATTAACTCGAACTGTTTGCCCACTCTATTTGCCCTGTACCAGAATGAACATTGAACTTTGGACTATACCTGAGGGGTGAGGTAAACAACAGGACTATAAATGGCCCGGGAGGCGCCCTTTGGA |
| | CCTTTTGCAATCCTGGCG |
| 321 | >CRM_SP0273 (CRM_LVR 133_V2) |
| | GGCGCCCTTTGGACCTTTTGCAATCCTGGAGCAAACAGCAAACACTGTACCAGAATGAACATTGAACTTTGGACTATACCTGAGGGGTGAGGTAAACAACAGGACTATAAATGGCCCGGGAGGCGCCCTTTGGACCTTTTGCAAT |
| | CCTGGCG |
| 322 | >CRM_SP0368 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCC |
| 323 | >CRM_SP0373 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTGTACCGGCCCGGGAGGCGCCCTTTGGACCTTTTGCAATCCTGGC |
| | G |
| 324 | >CRM_SP0378 |
| | TGCTCTCTGACAAAGATACGGTGGGTCCCACTGATGAACTGTGCTGCCACAGTAAATGTAGCCACTATGCCTATCTCCATTCTGAAGATGTGCCCTGTTCAAACATGTCCTAATACTCTGTCTCTGCAAGGGTCATCAGTAGTTT |
| | TCCATCTTACTCAACATCCTCCCAGTG |
| 325 | >CRM_SP0379 |
| | CCTCCCCGTGTTCCTGCTCTTTGTCCCTCTGTCCTACTTAGACTAATATTTGCCTTGGGTACTGCAAACAGGAAATGGGGGAGGGACAGGAGTAGGGCGGCCCTGTTCAAACATGTCCTAATACTCTGTCTCTGCAAGGGTCATC |
| | AGTAGTTTTCCATCTTACTCAACATCCTCCCAGTG |
| 326 | >CRM_SP0380 |
| | CCGCCCCCACTGAACCCTTGACCCCTGCCCTGCAGCCCCCGCAGCTTGCTGTTTGCCCACTCTATTTGCCCAGCCCCAGCCCTGTTCAAACATGTCCTAATACTCTGTCTCTGCAAGGGTCATCAGTAGTTTTCCATCTTACTCA |
| | ACATCCTCCCAGTG |
| 327 | >CRM_SP0381 |
| | AAGCTTTCTGAACAGCCAAACAGAGATTCCAAAGTTCAGGCACCAAAGTTCAGACCCTAACAGTTATTTACAAGGGTCAGTTAACCCCTGTTCAAACATGTCCTAATACTCTGTCTCTGCAAGGGTCATCAGTAGITTTCCATCT |
| | TACTCAACATCCTCCCAGTG |
| 328 | >CRM_SP0384 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCG |
| 329 | >CRM_SP0388 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCC |
| 330 | >CRM_SP0396 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTGTACCGGCCCGGGAGGCGCCCTTTGGACCTTTTGCAATCCTGGC |
| | GCCCTGGAGAGTCCTTTAGCAGGGCAAAGTGCAACATAGGCAGACCTTAAGGGATGACTCAGTAACAGATAAGCTTTGTGTGCCTGCA |
| 331 | >CRM_SP0397 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTGTACCCACTGAACCCTTGACCCCTGCCCTGCAGCCCCCGCAGCT |
| | TGCTGTTTGCCCACTCTATTTGCCCAGCCCCAGCCCTGGAGAGTCCTTTAGCAGGGCAAAGTGCAACATAGGCAGACCTTAAGGGATGACTCAGTAACAGATAAGCTTTGTGTGCCTGCA |
| 332 | >CRM SP0398 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTGAGGTTAATTTTTAAAAAGCAGTCAAAAGTCCAAGTGGCCCTT |
| | GGCAGCATTTACTCTCTCTGTTTGCTCTGGTTAATAATCTCAGGAGCACAAACATTCCTGTACCAGAATGAACATTGAACTTTGGACTATACCTGAGGGGTGAGGTAAACAACAGGACTATAAATGGCCCGGGAGGCGCCCTTTG |
| | GACCTTTTGCAATCCTGGCGCACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAACATAGGCAGACCTTAAGGGA |
| | TGACTCAGTAACAGATAAGCTTTGTGTGCCTGCA |
| 333 | >CRM_SP0399 |
| | AGAATGAACATTGAACTTTGGACTATACCTGAGGGGTGAGGTAAACAACAGGACTATAAAT |
| 334 | >CRM_SP0403 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTGAAGCAAATATTTGTGGTTATGGATTAACTCGAACTGTTTGCC |
| | CACTCTATTTGCCCTGTACCGGCCCGGGAGGCGCCCTTTGGACCTTTTGCAATCCTGGCGCACTGAACCCTTGACCCCTGCCCTGCAGCCCCCGCAGCTTGCTGTTTGCCCACTCTATTTGCCCAGCCCCAGCCCTGGAGAGTCC |
| | TTTAGCAGGGCAAAGTGCAACATAGGCAGACCTTAAGGGATGACTCAGTAACAGATAAGCTTTGTGTGCCTGCA |
| 335 | >CRM_SP0404 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTGAAGCAAATATTTGTGGTTATGGATTAACTCGAACTGTTTGCC |
| | CACTCTATTTGCCCTGTACCAGAATGAACATTGAACTTTGGACTATACCTGAGGGGTGAGGTAAACAACAGGACTATAAATGGCCCGGGAGGCGCCCTTTGGACCTTTTGCAATCCTGGCGCACTGAACCCTTGACCCCTGCCCT |
| | GCAGCCCCCGCAGCTTGCTGTTTGCCCACTCTATTTGCCCAGCCCCAGCCCTGGAGAGTCCTTTAGCAGGGCAAAGTGCAACATAGGCAGACCTTAAGGGATGACTCAGTAACAGATAAGCTTTGTGTGCCTGCA |
| 336 | >CRM_SP0405 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTGAAGCAAATATTTGTGGTTATGGATTAACTCGAACTGTTTGCC |
| | CACTCTATTTGCCCTGTACCAGAATGAACATTGAACTTTGGACTATACCTGAGGGGTGAGGTAAACAACAGGACTATAAATGGCCCGGGAGGCGCCCTTTGGACCTTTTGCAATCCTGGCGCACTGAACCCTTGACCCCTGCCCT |
| | GCAGCCCCCGCAGCTTGCTGTTTGCCCACTCTATTTGCCCAGCCCCAG |
| 337 | >CRM_SP0406 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCG |
| 338 | >CRM_SP0407 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCCAGGCTTTCACTTTCTCGCCAACTTACAAGGCCTTTCTGTGTAAA |
| | CAATACCTGAACCTTTACCCCGTTGCCCGGCAACGGCCAGGTCTGTGCCAAGTGTTTG |
| 339 | >CRM_SP0409 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCCCCTGTTCAAACATGTCCTAATACTCTGTCTCTGCAAGGGTCATC |
| | AGTAGTTTTCCATCTTACTCAACATCCTCCCAGTG |
| 340 | >CRM_SP0411 |
| | CCCTGTTCAAACATGTCCTAATACTCTGTCTCTGCAAGGGTCATCAGTAGTTTTCCATCTTACTCAACATCCTCCCAGTG |
| 86 | >CRM_SP0412 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCCCCTGTTCAAACATGTCCTAATACTCTGTCTCTGCAAGGGTCATC |
| | AGTAGTTTTCCATCTTACTCAACATCCTCCCAGTG (SEQ ID NO: 186) |
| 341 | >CRM_SP0413 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTGTACCGGCCCGGGAGGCGCCCTTTGGACCTTTTGCAATCCTGGC |
| | G |
| 342 | >SP0107 |
| | CCCTGGAGAGTCCTTTAGCAGGGCAAAGTGCAACATAGGCAGACCTTAAGGGATGACTCAGTAACAGATAAGCTTTGTGTGCCTGCAGGGGGATGGGAAGAGGGTGGGGCAGGAGAGGGACATAAAAGGGCTCTGAGGCATTGTA |
| | CTGTGAATTCCTTCAGTCTCCTGTTTGGAGAAGACAGAGCCAATGAGGCCCTCGTTCCAGGGAAACAGAATATGCTCAGCATGACGCAGCACTCCCTGAACTTTCCGGTTACATCACCCAATAGCTGAGATCAGAGGGCATATAA |
| | AACAGGGGCAAGGCACAGACTCATAGCAGAGCAATCACCACCAAGCCTGGAATAACTGCAGCCACC |
| 343 | >SP0109 |
| | CCTGGAGAGTCCTTTAGCAGGGCAAAGTGCAACATAGGCAGACCTTAAGGGATGACTCAGTAACAGATAAGCTTTGTGTGCCTGCAGGGGGATGGGAAGAGGGTGGGGCAGGAGAGGGACATAAAAGGGCTCTGAGGCATTGTAC |
| | TGTGAATTCCTTCAGTCTCCTGCTCTGCTCAGCCAGTCAGCCCTGCCTCCCTTGTTTAGGACCACACAGCACTGCTGGGTGTCTGCCTTTCCTTGGGTAATTTTTTTTTCTGGTTAATATTTAGCAAGAATTCTGCAGAGTGATC |
| | AAAAAAATCAAATACTCAGTATTTCAGAAATAGATTAAATAGGTTACTTTTTTACTGATAATGTGAAAGAATGATATAAAAACTTGATTTTCCTCAACAACATTACTTTCTTTTGTAAATGTGGTTTCTACAAAGATGAAACTAC |
| | TAAAACTTACAGGCCACC |
| 344 | >SP0111 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTTTGGAGAAGACAGAGCCAATGAGGCCCTCGTTCCAGGGAAACAG |
| | AATATGCTCAGCATGACGCAGCACTCCCTGAACTTTCCGGTTACATCACCCAATAGCTGAGATCAGAGGGCATATAAAACAGGGGCAAGGCACAGACTCATAGCAGAGCAATCACCACCAAGCCTGGAATAACTGCAGCCACC |
| 345 | >SP0112 |
| | CACTGAACCCTTGACCCCTGCCCTGCAGCCCCCGCAGCTTGCTGTTTGCCCACTCTATTTGCCCAGCCCCAGAGGTTAATTTTTAAAAAGCAGTCAAAAGTCCAAGTGGCCCTTGGCAGCATTTACTCTCTCTGTTTGCTCTGGT |
| | TAATAATCTCAGGAGCACAAACATTCCTTTGGAGAAGACAGAGCCAATGAGGCCCTCGTTCCAGGGAAACAGAATATGCTCAGCATGACGCAGCACTCCCTGAACTTTCCGGTTACATCACCCAATAGCTGAGATCAGAGGGCAT |
| | ATAAAACAGGGGCAAGGCACAGACTCATAGCAGAGCAATCACCACCAAGCCTGGAATAACTGCAGCCACC |
| 346 | >SP0113 |
| | CACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAACATAGGCAGACCTTAAGGGATGACTCAGTAACAGATAAGC |
| | TTTGTGTGCCTGCAGGGGGATGGGAAGAGGGTGGGGCAGGAGAGGGACATAAAAGGGCTCTGAGGCATTGTACTGTGAATTCCTTCAGTCTCCTGGGGCATATAAAACAGGGGCAAGGCACAGACTCATAGCAGAGCAATCACCA |
| | CCAAGCCTGGAATAACTGCAGCCACC |
| 347 | >SP0115 |
| | CCCTGGAGAGTCCTTTAGCAGGGCAAAGTGCAACATAGGCAGACCTTAAGGGATGACTCAGTAACAGATAAGCTTTGTGTGCCTGCAGGGGGATGGGAAGAGGGTGGGGCAGGAGAGGGACATAAAAGGGCTCTGAGGCATTGTA |
| | CTGTGAATTCCTTCAGTCTCCTGCTCTGCTCAGCCAGTCAGCCCTGCCTCCCTTGTTTAGGACCACACAGCACTGCTGGGTGTCTGCCTTTCCTTGTGGACTTAGCCCCTGTTTGCTCCTCCGATAACTGGGGTGACCTTGGTTA |
| | ATATTCACCAGCAGCCTCCCCCGTTGCCCCTCTGGATCCACTGCTTAAATACGGACGAGGACAGGGCCCTGTCTCCTCAGCTTCAGGCACCACCACTGACCTGGGACAGTGAATCGCCACC |
| 348 | >SP0116 |
| | CCCTGGAGAGTCCTTTAGCAGGGCAAAGTGCAACATAGGCAGACCTTAAGGGATGACTCAGTAACAGATAAGCTTTGTGTGCCTGCAGGGGGATGGGAAGAGGGTGGGGCAGGAGAGGGACATAAAAGGGCTCTGAGGCATTGTA |
| | CTGTGAATTCCTTCAGTCTCCTGTGGACTTAGCCCCTGTTTGCTCCTCCGATAACTGGGGTGACCTTGGTTAATATTCACCAGCAGCCTCCCCCGTTGCCCCTCTGGATCCACTGCTTAAATACGGACGAGGACAGGGCCCTGTC |
| | TCCTCAGCTTCAGGCACCACCACTGACCTGGGACAGTGAATCGCCACC |
| 349 | >SP0121 |
| | CCCTGGAGAGTCCTTTAGCAGGGCAAAGTGCAACATAGGCAGACCTTAAGGGATGACTCAGTAACAGATAAGCTTTGTGTGCCTGCACCCTGGAGAGTCCTTTAGCAGGGCAAAGTGCAACATAGGCAGACCTTAAGGGATGACT |
| | CAGTAACAGATAAGCTTTGTGTGCCTGCAGGGCATATAAAACAGGGGCAAGGCACAGACTCATAGCAGAGCAATCACCACCAAGCCTGGAATAACTGCAGCCACC |
| 350 | >SP0124 |
| | CACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAACATAGGCAGACCTTAAGGGATGACTCAGTAACAGATAAGC |
| | TTTGTGTGCCTGCACTCTTTTGTTTTACATGAAGGGTCTGGCAGCCAAAGCAATCACTCAAAGTTCAAACCTTATCATTTTTTGCTTTGTTCCTCTTGGCCTTGGTTTTGTACATCAGCTTTGAAAATACCATCCCAGGGTTAAT |
| | GCTGGGGTTAATTTATAACTAAGAGTGCTCTAGTTTTGCAATACAGGACATGCTATAAAAATGGAAAGATGTTGCTTTCTGAGAGATGCGCCACC |
| 351 | >SP0127 (LVR_SP127) |
| | AGAATGAACATTGAACTTTGGACTATACCTGAGGGGTGAGGTAAACAACAGGACTATAAATGGCCCGGGAGGCGCCCTTTGGACCTTTTGCAATCCTGGCGCACTGAACCCTTGACCCCTGCCCTGCAGCCCCCGCAGCTTGCTG |
| | TTTGCCCACTCTATTTGCCCAGCCCCAGGGACAGAGCTGATCCTTGAACTCTTAAGTTCCACGGGCATATAAAACAGGGGCAAGGCACAGACTCATAGCAGAGCAATCACCACCAAGCCTGGAATAACTGCAGCCACC |
| 352 | >SP0127A1 (LVR_SP127_A1) |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTGTACCAGAATGAACATTGAACTTTGGACTATACCTGAGGGGTGA |
| | GGTAAACAACAGGACTATAAATGGCCCGGGAGGCGCCCTTTGGACCTTTTGCAATCCTGGCGCACTGAACCCTTGACCCCTGCCCTGCAGCCCCCGCAGCTTGCTGTTTGCCCACTCTATTTGCCCAGCCCCAGGGACAGAGCTG |
| | ATCCTTGAACTCTTAAGTTCCACGGGCATATAAAACAGGGGCAAGGCACAGACTCATAGCAGAGCAATCACCACCAAGCCTGGAATAACTGCAGCCACC |
| 353 | >SP0127V1 (LVR_SP127_V1) |
| | AAGCAAATATTTGTGGTTATGGATTAACTCGAACTGTTTGCCCACTCTATTTGCCCTGTACCAGAATGAACATTGAACTTTGGACTATACCTGAGGGGTGAGGTAAACAACAGGACTATAAATGGCCCGGGAGGCGCCCTTTGGA |
| | CCTTTTGCAATCCTGGCGCACTGAACCCTTGACCCCTGCCCTGCAGCCCCCGCAGCTTGCTGTTTGCCCACTCTATTTGCCCAGCCCCAGGGACAGAGCTGATCCTTGAACTCTTAAGTTCCACGGGCATATAAAACAGGGGCAA |
| | GGCACAGACTCATAGCAGAGCAATCACCACCAAGCCTGGAATAACTGCAGCCACC |
| 354 | >SP0127V2 (LVR_SP127_V2) |
| | GGCGCCCTTTGGACCTTTTGCAATCCTGGAGCAAACAGCAAACACTGTACCAGAATGAACATTGAACTTTGGACTATACCTGAGGGGTGAGGTAAACAACAGGACTATAAATGGCCCGGGAGGCGCCCTTTGGACCTTTTGCAAT |
| | CCTGGCGCACTGAACCCTTGACCCCTGCCCTGCAGCCCCCGCAGCTTGCTGTTTGCCCACTCTATTTGCCCAGCCCCAGGGACAGAGCTGATCCTTGAACTCTTAAGTTCCACGGGCATATAAAACAGGGGCAAGGCACAGACTC |
| | ATAGCAGAGCAATCACCACCAAGCCTGGAATAACTGCAGCCACC |
| 355 | >SP0128 |
| | AGAATGAACATTGAACTTTGGACTATACCTGAGGGGTGAGGTAAACAACAGGACTATAAATAGAATGAACATTGAACTTTGGACTATACCTGAGGGGTGAGGTAAACAACAGGACTATAAATCACTGAACCCTTGACCCCTGCCC |
| | TGCAGCCCCCGCAGCTTGCTGTTTGCCCACTCTATTTGCCCAGCCCCAGGGGCATATAAAACAGGGGCAAGGCACAGACTCATAGCAGAGCAATCACCACCAAGCCTGGAATAACTGCAGCCACC |
| 356 | >SP0131 (LVR_SP131) |
| | GGCCCGGGAGGCGCCCTTTGGACCTTTTGCAATCCTGGCGCACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAA |
| | CATAGGCAGACCTTAAGGGATGACTCAGTAACAGATAAGCTTTGTGTGCCTGCAGGGCATATAAAACAGGGGCAAGGCACAGACTCATAGCAGAGCAATCACCACCAAGCCTGGAATAACTGCAGCCACC |
| 357 | >SP0132 (LVR_SP132) |
| | AGAATGAACATTGAACTTTGGACTATACCTGAGGGGTGAGGTAAACAACAGGACTATAAATGGCCCGGGAGGCGCCCTTTGGACCTTTTGCAATCCTGGCGCACTGAACCCTTGACCCCTGCCCTGCAGCCCCCGCAGCTTGCTG |
| | TTTGCCCACTCTATTTGCCCAGCCCCAGCCCTGGAGAGTCCTTTAGCAGGGCAAAGTGCAACATAGGCAGACCTTAAGGGATGACTCAGTAACAGATAAGCTTTGTGTGCCTGCAGGGCATATAAAACAGGGGCAAGGCACAGAC |
| | TCATAGCAGAGCAATCACCACCAAGCCTGGAATAACTGCAGCCACC |
| 358 | >SP0133 (LVR_SP133) |
| | AGAATGAACATTGAACTTTGGACTATACCTGAGGGGTGAGGTAAACAACAGGACTATAAATGGCCCGGGAGGCGCCCTTTGGACCTTTTGCAATCCTGGCGCTCTTTTGTTTTACATGAAGGGTCTGGCAGCCAAAGCAATCACT |
| | CAAAGTTCAAACCTTATCATTTTTTGCTTTGTTCCTCTTGGCCTTGGTTTTGTACATCAGCTTTGAAAATACCATCCCAGGGTTAATGCTGGGGTTAATTTATAACTAAGAGTGCTCTAGTTTTGCAATACAGGACATGCTATAA |
| | AAATGGAAAGATGTTGCTTTCTGAGAGATGCGCCACC |
| 359 | >SP0155 |
| | GCTGGTTTCTTATAAAACTGATGGAAGATACAAACACTATTAAAGAACTGTTTGCATGTTGCAAATGATGTCCAAAGTCCAAACATTGTTAATAATTAATACTCCAATAAACATCATGTCAGAATTTCTGTTTTCTTTTCCCTTT |
| | GAACCTTTGCAGGATTGCCACATCATCAGGACCACACCTTCATCAGGAATGAATATCCGATGACCTAATGATTCTGAGCTTGGCAAAGGTCTTATCTCCCAGCTCGCCCAGGCCCAGTGTTCCAGGAATGTGACCTTTGCTGCAG |
| | CAGCCGCTGGAGGGGGCAGAGGGGATGGGCTGGAGGTTGAGCAAACAGAGCAGCAGAAAAGGCAGTTCCTCTTCTCCAGTGCCCTCCTTCCCTGTCTCTGCCTCTCCCTCCCTTCCTCAGGCATCAGAGCGGAGACTTCAGGGAG |
| | ACCAGAGCCCAGCTTGCCAGGCACTGAGCTAGAAGCCCTGCCATG |
| 360 | >SP0158 |
| | ATTGGCATCTTCTATTGCTTTTCCTGGTGACTTCATTTTTCACTCTTGGCTAAAAATGGGTCTCTGATGATTTATTCTATCCTGGGTGTTGACAAGCTGAAGAAGTTGTGTGGGGCCTGCTGCCAGTAACCCTGGGTGACGAAGC |
| | GTGACTCACCACTCCGAGGTCAGTGGGGGGATGGAAGGCAGGGGAGTCAGCTGACAAGATCTGCTGCTTTGTCACCAGGCCTTCTGCCCGATGACCTAATGATTCTGAGCTTGGCAAAGGTCTTATCTCCCAGCTCGCCCAGGCC |
| | CAGTGTTCCAGGAATGTGACCTTTGCTGCAGCAGCCGCTGGAGGGGGCAGAGGGGATGGGCTGGAGGTTGAGCAAACAGAGCAGCAGAAAAGGCAGTTCCTCTTCTCCAGTGCCCTCCTTCCCTGTCTCTGCCTCTCCCTCCCTT |
| | CCTCAGGCATCAGAGCGGAGACTTCAGGGAGACCAGAGCCCAGCTTGCCAGGCACTGAGCTAGAAGCCCTGCCATG |
| 361 | >SP0163 |
| | TGAAATGCCTGCCATATATTAGTGCCCTGAAGTCCAAAGGTAGAGGAACCGAGTGTTTAAAAATTACTGTGGCTGTGGAGTCAACATGATGTAAAAAAACAAACATTTGGATAACACCAAGAAGCCAGATATGGTTGAAATGTTG |
| | ACTGGTTGACAAAAATAATTTGGGTTGCTTAATGGTGCACAAAGGTAATGCAAAACCGATGACCTAATGATTCTGAGCTTGGCAAAGGTCTTATCTCCCAGCTCGCCCAGGCCCAGTGTTCCAGGAATGTGACCTTTGCTGCAGC |
| | AGCCGCTGGAGGGGGCAGAGGGGATGGGCTGGAGGTTGAGCAAACAGAGCAGCAGAAAAGGCAGTTCCTCTTCTCCAGTGCCCTCCTTCCCTGTCTCTGCCTCTCCCTCCCTTCCTCAGGCATCAGAGCGGAGACTTCAGGGAGA |
| | CCAGAGCCCAGCTTGCCAGGCACTGAGCTAGAAGCCCTGCCATG |
| 362 | >SP0236 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTGCCTTTGCAACAGCTTATCGGAAGCAAACAAGCTGAGGGGAAT |
| | TGAGCAAGAATTTCTGGGATACCAACAGCATAGGAGGAACAAAGGACGTAGAGGGAGGGTTGACTGTCTACACAGGACAAAGCCAATGATTAACCAAACCTCTTGCAGATTTAAATAGGATGGGAACTAGGAGTGGCAGCAATCC |
| | TTTCTTTCAGCTGGAGTGCTCCTCAGGAGCCAGCCCCACCCTTAGAAAAG |
| 93 | >SP0239 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTGAGGTTAATTTTTAAAAAGCAGTCAAAAGTCCAAGTGGCCCTT |
| | GGCAGCATTTACTCTCTCTGTTTGCTCTGGTTAATAATCTCAGGAGCACAAACATTCCGGCCCGGGAGGCGCCCTTTGGACCTTTTGCAATCCTGGCGCACTGAACCCTTGACCCCTGCCCTGCAGCCCCCGCAGCTTGCTGTTT |
| | GCCCACTCTATTTGCCCAGCCCCAGCCCTGGAGAGTCCTTTAGCAGGGCAAAGTGCAACATAGGCAGACCTTAAGGGATGACTCAGTAACAGATAAGCTTTGTGTGCCTGCAGGGCATATAAAACAGGGGCAAGGCACAGACTCA |
| | TAGCAGAGCAATCACCACCAAGCCTGGAATAACTGCAGCCACC |
| 95 | >SP0240 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTGCCGATGACCTAATGATTCTGAGCTTGGCAAAGGTCTTATCTC |
| | CCAGCTCGCCCAGGCCCAGTGTTCCAGGAATGTGACCTTTGCTGCAGCAGCCGCTGGAGGGGGCAGAGGGGATGGGCTGGAGGTTGAGCAAACAGAGCAGCAGAAAAGGCAGTTCCTCTTCTCCAGTGCCCTCCTTCCCTGTCTC |
| | TGCCTCTCCCTCCCTTCCTCAGGCATCAGAGCGGAGACTTCAGGGAGACCAGAGCCCAGCTTGCCAGGCACTGAGCTAGAAGCCCTGCC |
| 363 | >SP0241 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGCACTG |
| | AACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAACATAGGCAGACCTTAAGGGATGACTCAGTAACAGATAAGCTTTGT |
| | GTGCCTGCACCGATGACCTAATGATTCTGAGCTTGGCAAAGGTCTTATCTCCCAGCTCGCCCAGGCCCAGTGTTCCAGGAATGTGACCTTTGCTGCAGCAGCCGCTGGAGGGGGCAGAGGGGATGGGCTGGAGGTTGAGCAAACA |
| | GAGCAGCAGAAAAGGCAGTTCCTCTTCTCCAGTGCCCTCCTTCCCTGTCTCTGCCTCTCCCTCCCTTCCTCAGGCATCAGAGCGGAGACTTCAGGGAGACCAGAGCCCAGCTTGCCAGGCACTGAGCTAGAAGCCCTGCC |
| 364 | >SP0242 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGCACTG |
| | AACCCTTGACCCCTGCCCTGCAGCCCCCGCAGCTTGCTGTTTGCCCACTCTATTTGCCCAGCCCCAGCCGATGACCTAATGATTCTGAGCTTGGCAAAGGTCTTATCTCCCAGCTCGCCCAGGCCCAGTGTTCCAGGAATGTGAC |
| | CTTTGCTGCAGCAGCCGCTGGAGGGGGCAGAGGGGATGGGCTGGAGGTTGAGCAAACAGAGCAGCAGAAAAGGCAGTTCCTCTTCTCCAGTGCCCTCCTTCCCTGTCTCTGCCTCTCCCTCCCTTCCTCAGGCATCAGAGCGGAG |
| | ACTTCAGGGAGACCAGAGCCCAGCTTGCCAGGCACTGAGCTAGAAGCCCTGCC |
| 365 | >SP0243 |
| | TGAAATGCCTGCCATATATTAGTGCCCTGAAGTCCAAAGGTAGAGGAACCGAGTGTTTAAAAATTACTGTGGCTGTGGAGTCAACATGATGTAAAAAAACAAACATTTGGATAACACCAAGAAGCCAGATATGGTTGAAATGTTG |
| | ACTGGTTGACAAAAATAATTTGGGTTGCTTAATGGTGCACAAAGGTAATGCAAAAAGGTTAATTTTTAAAAAGCAGTCAAAAGTCCAAGTGGCCCTTGGCAGCATTTACTCTCTCTGTTTGCTCTGGTTAATAATCTCAGGAGCA |
| | CAAACATTCCGGCCCGGGAGGCGCCCTTTGGACCTTTTGCAATCCTGGCGCACTGAACCCTTGACCCCTGCCCTGCAGCCCCCGCAGCTTGCTGTTTGCCCACTCTATTTGCCCAGCCCCAGCCCTGGAGAGTCCTTTAGCAGGG |
| | CAAAGTGCAACATAGGCAGACCTTAAGGGATGACTCAGTAACAGATAAGCTTTGTGTGCCTGCAGGGCATATAAAACAGGGGCAAGGCACAGACTCATAGCAGAGCAATCACCACCAAGCCTGGAATAACTGCAGCCACC |
| 366 | >SP0244 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGCACTG |
| | AACCCTTGACCCCTGCCCTGCAGCCCCCGCAGCTTGCTGTTTGCCCACTCTATTTGCCCAGCCCCAGTGAAATGCCTGCCATATATTAGTGCCCTGAAGTCCAAAGGTAGAGGAACCGAGTGTTTAAAAATTACTGTGGCTGTGG |
| | AGTCAACATGATGTAAAAAAACAAACATTTGGATAACACCAAGAAGCCAGATATGGTTGAAATGTTGACTGGTTGACAAAAATAATTTGGGTTGCTTAATGGTGCACAAAGGTAATGCAAAACCGATGACCTAATGATTCTGAGC |
| | TTGGCAAAGGTCTTATCTCCCAGCTCGCCCAGGCCCAGTGTTCCAGGAATGTGACCTTTGCTGCAGCAGCCGCTGGAGGGGGCAGAGGGGATGGGCTGGAGGTTGAGCAAACAGAGCAGCAGAAAAGGCAGTTCCTCTTCTCCAG |
| | TGCCCTCCTTCCCTGTCTCTGCCTCTCCCTCCCTTCCTCAGGCATCAGAGCGGAGACTTCAGGGAGACCAGAGCCCAGCTTGCCAGGCACTGAGCTAGAAGCCCTGCC |
| 96 | >SP0246 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGCACTG |
| | AACCCTTGACCCCTGCCCTGCAGCCCCCGCAGCTTGCTGTTTGCCCACTCTATTTGCCCAGCCCCAGGGGCATATAAAACAGGGGCAAGGCACAGACTCATAGCAGAGCAATCACCACCAAGCCTGGAATAACTGCAGCCACC |
| 367 | >SP0247 |
| | GCTGGTTTCTTATAAAACTGATGGAAGATACAAACACTATTAAAGAACTGTTTGCATGTTGCAAATGATGTCCAAAGTCCAAACATTGTTAATAATTAATACTCCAATAAACATCATGTCAGAATTTCTGTTTTCTTTTCCCTTT |
| | GAACCTTTGCAGGATTGCCACATCATCAGGACCACACCTTCATCAGGAATGAATATCAGGCTTTCACTTTCTCGCCAACTTACAAGGCCTTTCTGTGTAAACAATACCTGAACCTTTACCCCGTTGCCCGGCAACGGCCAGGTCT |
| | GTGCCAAGTGTTTGCCGATGACCTAATGATTCTGAGCTTGGCAAAGGTCTTATCTCCCAGCTCGCCCAGGCCCAGTGTTCCAGGAATGTGACCTTTGCTGCAGCAGCCGCTGGAGGGGGCAGAGGGGATGGGCTGGAGGTTGAGC |
| | AAACAGAGCAGCAGAAAAGGCAGTTCCTCTTCTCCAGTGCCCTCCTTCCCTGTCTCTGCCTCTCCCTCCCTTCCTCAGGCATCAGAGCGGAGACTTCAGGGAGACCAGAGCCCAGCTTGCCAGGCACTGAGCTAGAAGCCCTGCC |
| 368 | >SP0248 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGCACTG |
| | AACCCTTGACCCCTGCCCTGCAGCCCCCGCAGCTTGCTGTTTGCCCACTCTATTTGCCCAGCCCCAGCAGGCTTTCACTTTCTCGCCAACTTACAAGGCCTTTCTGTGTAAACAATACCTGAACCTTTACCCCGTTGCCCGGCAA |
| | CGGCCAGGTCTGTGCCAAGTGTTTGGGGCATATAAAACAGGGGCAAGGCACAGACTCATAGCAGAGCAATCACCACCAAGCCTGGAATAACTGCAGCCACC |
| 369 | >SP0249 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGCACTG |
| | AACCCTTGACCCCTGCCCTGCAGCCCCCGCAGCTTGCTGTTTGCCCACTCTATTTGCCCAGCCCCAGCAGGCTTTCACTTTCTCGCCAACTTACAAGGCCTTTCTGTGTAAACAATACCTGAACCTTTACCCCGTTGCCCGGCAA |
| | CGGCCAGGTCTGTGCCAAGTGTTTGGGGCGACTCAGATCCCAGCCAGTGGACTTAGCCCCTGTTTGCTCCTCCGATAACTGGGGTGACCTTGGTTAATATTCACCAGCAGCCTCCCCCGTTGCCCCTCTGGATCCACTGCTTAAA |
| | TACGGACGAGGACAGGGCCCTGTCTCCTCAGCTTCAGGCACCACCACTGACCTGGGACAGTGAATCGCCACC |
| 370 | >SP0250 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGCACTG |
| | AACCCTTGACCCCTGCCCTGCAGCCCCCGCAGCTTGCTGTTTGCCCACTCTATTTGCCCAGCCCCAGGCTGGTTTCTTATAAAACTGATGGAAGATACAAACACTATTAAAGAACTGTTTGCATGTTGCAAATGATGTCCAAAGT |
| | CCAAACATTGTTAATAATTAATACTCCAATAAACATCATGTCAGAATTTCTGTTTTCTTTTCCCTTTGAACCTTTGCAGGATTGCCACATCATCAGGACCACACCTTCATCAGGAATGAATATCCGATGACCTAATGATTCTGAG |
| | CTTGGCAAAGGTCTTATCTCCCAGCTCGCCCAGGCCCAGTGTTCCAGGAATGTGACCTTTGCTGCAGCAGCCGCTGGAGGGGGCAGAGGGGATGGGCTGGAGGTTGAGCAAACAGAGCAGCAGAAAAGGCAGTTCCTCTTCTCCA |
| | GTGCCCTCCTTCCCTGTCTCTGCCTCTCCCTCCCTTCCTCAGGCATCAGAGCGGAGACTTCAGGGAGACCAGAGCCCAGCTTGCCAGGCACTGAGCTAGAAGCCCTGCC |
| 371 | >SP0251 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGCACTG |
| | AACCCTTGACCCCTGCCCTGCAGCCCCCGCAGCTTGCTGTTTGCCCACTCTATTTGCCCAGCCCCAGAAGCAAATATTTGTGGTTATGGATTAACTCGAACTGTTTGCCCACTCTATTTGCCCGGGCATATAAAACAGGGGCAAG |
| | GCACAGACTCATAGCAGAGCAATCACCACCAAGCCTGGAATAACTGCAGCCACC |
| 372 | >SP0252 |
| | AAGCAAATATTTGTGGTTATGGATTAACTCGAACTGTTTGCCCACTCTATTTGCCCCAGGCTTTCACTTTCTCGCCAACTTACAAGGCCTTTCTGTGTAAACAATACCTGAACCTTTACCCCGTTGCCCGGCAACGGCCAGGTCT |
| | GTGCCAAGTGTTTGCCTTTGCAACAGCTTATCGGAAGCAAACAAGCTGAGGGGAATTGAGCAAGAATTTCTGGGATACCAACAGCATAGGAGGAACAAAGGACGTAGAGGGAGGGTTGACTGTCTACACAGGACAAAGCCAATGA |
| | TTAACCAAACCTCTTGCAGATTTAAATAGGATGGGAACTAGGAGTGGCAGCAATCCTTTCTTTCAGCTGGAGTGCTCCTCAGGAGCCAGCCCCACCCTTAGAAAAGCCACC |
| 373 | >SP0253 |
| | AAGCAAATATTTGTGGTTATGGATTAACTCGAACTGTTTGCCCACTCTATTTGCCCCAGGCTTTCACTTTCTCGCCAACTTACAAGGCCTTTCTGTGTAAACAATACCTGAACCTTTACCCCGTTGCCCGGCAACGGCCAGGTCT |
| | GTGCCAAGTGTTTGCCGATGACCTAATGATTCTGAGCTTGGCAAAGGTCTTATCTCCCAGCTCGCCCAGGCCCAGTGTTCCAGGAATGTGACCTTTGCTGCAGCAGCCGCTGGAGGGGGCAGAGGGGATGGGCTGGAGGTTGAGC |
| | AAACAGAGCAGCAGAAAAGGCAGTTCCTCTTCTCCAGTGCCCTCCTTCCCTGTCTCTGCCTCTCCCTCCCTTCCTCAGGCATCAGAGCGGAGACTTCAGGGAGACCAGAGCCCAGCTTGCCAGGCACTGAGCTAGAAGCCCTGCC |
| 374 | >SP0254 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGCAGGC |
| | TTTCACTTTCTCGCCAACTTACAAGGCCTTTCTGTGTAAACAATACCTGAACCTTTACCCCGTTGCCCGGCAACGGCCAGGTCTGTGCCAAGTGTTTGCCTTTGCAACAGCTTATCGGAAGCAAACAAGCTGAGGGGAATTGAGC |
| | AAGAATTTCTGGGATACCAACAGCATAGGAGGAACAAAGGACGTAGAGGGAGGGTTGACTGTCTACACAGGACAAAGCCAATGATTAACCAAACCTCTTGCAGATTTAAATAGGATGGGAACTAGGAGTGGCAGCAATCCTTTCT |
| | TTCAGCTGGAGTGCTCCTCAGGAGCCAGCCCCACCCTTAGAAAAGCCACC |
| 375 | >SP0255 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGCAGGC |
| | TTTCACTTTCTCGCCAACTTACAAGGCCTTTCTGTGTAAACAATACCTGAACCTTTACCCCGTTGCCCGGCAACGGCCAGGTCTGTGCCAAGTGTTTGCCGATGACCTAATGATTCTGAGCTTGGCAAAGGTCTTATCTCCCAGC |
| | TCGCCCAGGCCCAGTGTTCCAGGAATGTGACCTTTGCTGCAGCAGCCGCTGGAGGGGGCAGAGGGGATGGGCTGGAGGTTGAGCAAACAGAGCAGCAGAAAAGGCAGTTCCTCTTCTCCAGTGCCCTCCTTCCCTGTCTCTGCCT |
| | CTCCCTCCCTTCCTCAGGCATCAGAGCGGAGACTTCAGGGAGACCAGAGCCCAGCTTGCCAGGCACTGAGCTAGAAGCCCTGCC |
| 376 | >SP0256 |
| | ATTGGCATCTTCTATTGCTTTTCCTGGTGACTTCATTTTTCACTCTTGGCTAAAAATGGGTCTCTGATGATTTATTCTATCCTGGGTGTTGACAAGCTGAAGAAGTTGTGTGGGGCCTGCTGCCAGTAACCCTGGGTGACGAAGC |
| | GTGACTCACCACTCCGAGGTCAGTGGGGGGATGGAAGGCAGGGGAGTCAGCTGACAAGATCTGCTGCTTTGTCACCAGGCCTTCTGCCAGGCTTTCACTTTCTCGCCAACTTACAAGGCCTTTCTGTGTAAACAATACCTGAACC |
| | TTTACCCCGTTGCCCGGCAACGGCCAGGTCTGTGCCAAGTGTTTGCCGATGACCTAATGATTCTGAGCTTGGCAAAGGTCTTATCTCCCAGCTCGCCCAGGCCCAGTGTTCCAGGAATGTGACCTTTGCTGCAGCAGCCGCTGGA |
| | GGGGGCAGAGGGGATGGGCTGGAGGTTGAGCAAACAGAGCAGCAGAAAAGGCAGTTCCTCTTCTCCAGTGCCCTCCTTCCCTGTCTCTGCCTCTCCCTCCCTTCCTCAGGCATCAGAGCGGAGACTTCAGGGAGACCAGAGCCCA |
| | GCTTGCCAGGCACTGAGCTAGAAGCCCTGCC |
| 377 | >SP0257 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTGGCTGGTTTCTTATAAAACTGATGGAAGATACAAACACTATTA |
| | AAGAACTGTTTGCATGTTGCAAATGATGTCCAAAGTCCAAACATTGTTAATAATTAATACTCCAATAAACATCATGTCAGAATTTCTGTTTTCTTTTCCCTTTGAACCTTTGCAGGATTGCCACATCATCAGGACCACACCTTCA |
| | TCAGGAATGAATATCCGATGACCTAATGATTCTGAGCTTGGCAAAGGTCTTATCTCCCAGCTCGCCCAGGCCCAGTGTTCCAGGAATGTGACCTTTGCTGCAGCAGCCGCTGGAGGGGGCAGAGGGGATGGGCTGGAGGTTGAGC |
| | AAACAGAGCAGCAGAAAAGGCAGTTCCTCTTCTCCAGTGCCCTCCTTCCCTGTCTCTGCCTCTCCCTCCCTTCCTCAGGCATCAGAGCGGAGACTTCAGGGAGACCAGAGCCCAGCTTGCCAGGCACTGAGCTAGAAGCCCTGCC |
| 378 | >SP0258 |
| | CTGTTTGCTGCTTGCAATGTTTGCCCATTTTAGGGAGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAG |
| | GCGCCCTTTGGACCTTTTGCAATCCTGGCGCACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAACATAGGCAGA |
| | CCTTAAGGGATGACTCAGTAACAGATAAGCTTTGTGTGCCTGCAGGGCATATAAAACAGGGGCAAGGCACAGACTCATAGCAGAGCAATCACCACCAAGCCTGGAATAACTGCAGCCACC |
| 379 | >SP0259 |
| | CTGTTTGCTGCTTGCAATGTTTGCCCATTTTAGGGGCTGGTTTCTTATAAAACTGATGGAAGATACAAACACTATTAAAGAACTGTTTGCATGTTGCAAATGATGTCCAAAGTCCAAACATTGTTAATAATTAATACTCCAATAA |
| | ACATCATGTCAGAATTTCTGTTTTCTTTTCCCTTTGAACCTTTGCAGGATTGCCACATCATCAGGACCACACCTTCATCAGGAATGAATATCCGATGACCTAATGATTCTGAGCTTGGCAAAGGTCTTATCTCCCAGCTCGCCCA |
| | GGCCCAGTGTTCCAGGAATGTGACCTTTGCTGCAGCAGCCGCTGGAGGGGGCAGAGGGGATGGGCTGGAGGTTGAGCAAACAGAGCAGCAGAAAAGGCAGTTCCTCTTCTCCAGTGCCCTCCTTCCCTGTCTCTGCCTCTCCCTC |
| | CCTTCCTCAGGCATCAGAGCGGAGACTTCAGGGAGACCAGAGCCCAGCTTGCCAGGCACTGAGCTAGAAGCCCTGCC |
| 380 | >SP0264 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTGCCTTTGCAACAGCTTATCGGAAGCAAACAAGCTGAGGGGAAT |
| | TGAGCAAGAATTTCTGGGATACCAACAGCATAGGAGGAACAAAGGACGTAGAGGGAGGGTTGACTGTCTACACAGGACAAAGCCAATGATTAACCAAACCTCTTGCAGATTTAAATAGGATGGGAACTAGGAGTGGCAGCAATCC |
| | TTTCTTTCAGCTGGAGTGCTCCTCAGGAGCCAGCCCCACCCTTAGAAAAGCCACC |
| 94 | >SP0265 (LVR_SP131_A1) |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTGTACCGGCCCGGGAGGCGCCCTTTGGACCTTTTGCAATCCTGGC |
| | GCACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAACATAGGCAGACCTTAAGGGATGACTCAGTAACAGATAAG |
| | CTTTGTGTGCCTGCAGGGCATATAAAACAGGGGCAAGGCACAGACTCATAGCAGAGCAATCACCACCAAGCCTGGAATAACTGCAGCCACC |
| 381 | >SP0266 (LVR_SP131_V1) |
| | AAGCAAATATTTGTGGTTATGGATTAACTCGAACTGTTTGCCCACTCTATTTGCCCTGTACCGGCCCGGGAGGCGCCCTTTGGACCTTTTGCAATCCTGGCGCACTGAACCCTTGACCCCTGCCCTGCAGCCCCCGCAGCTTGCT |
| | GTTTGCCCACTCTATTTGCCCAGCCCCAGCCCTGGAGAGTCCTTTAGCAGGGCAAAGTGCAACATAGGCAGACCTTAAGGGATGACTCAGTAACAGATAAGCTTTGTGTGCCTGCAGGGCATATAAAACAGGGGCAAGGCACAGA |
| | CTCATAGCAGAGCAATCACCACCAAGCCTGGAATAACTGCAGCCACC |
| 382 | >SP0267 (LVR_SP131_V2) |
| | GGCGCCCTTTGGACCTTTTGCAATCCTGGAGCAAACAGCAAACACTGTACCGGCCCGGGAGGCGCCCTTTGGACCTTTTGCAATCCTGGCGCACTGAACCCTTGACCCCTGCCCTGCAGCCCCCGCAGCTTGCTGTTTGCCCACT |
| | CTATTTGCCCAGCCCCAGCCCTGGAGAGTCCTTTAGCAGGGCAAAGTGCAACATAGGCAGACCTTAAGGGATGACTCAGTAACAGATAAGCTTTGTGTGCCTGCAGGGCATATAAAACAGGGGCAAGGCACAGACTCATAGCAGA |
| | GCAATCACCACCAAGCCTGGAATAACTGCAGCCACC |
| 383 | >SP0268 (LVR 132_A1) |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTGTACCAGAATGAACATTGAACTTTGGACTATACCTGAGGGGTGA |
| | GGTAAACAACAGGACTATAAATGGCCCGGGAGGCGCCCTTTGGACCTTTTGCAATCCTGGCGCACTGAACCCTTGACCCCTGCCCTGCAGCCCCCGCAGCTTGCTGTTTGCCCACTCTATTTGCCCAGCCCCAGCCCTGGAGAGT |
| | CCTTTAGCAGGGCAAAGTGCAACATAGGCAGACCTTAAGGGATGACTCAGTAACAGATAAGCTTTGTGTGCCTGCAGGGCATATAAAACAGGGGCAAGGCACAGACTCATAGCAGAGCAATCACCACCAAGCCTGGAATAACTGC |
| | AGCCACC |
| 384 | >SP0269 (LVR_132_V1) |
| | AAGCAAATATTTGTGGTTATGGATTAACTCGAACTGTTTGCCCACTCTATTTGCCCTGTACCAGAATGAACATTGAACTTTGGACTATACCTGAGGGGTGAGGTAAACAACAGGACTATAAATGGCCCGGGAGGCGCCCTTTGGA |
| | CCTTTTGCAATCCTGGCGCACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAACATAGGCAGACCTTAAGGGATG |
| | ACTCAGTAACAGATAAGCTTTGTGTGCCTGCAGGGCATATAAAACAGGGGCAAGGCACAGACTCATAGCAGAGCAATCACCACCAAGCCTGGAATAACTGCAGCCACC |
| 385 | >SP0270 (LVR 132_V2) |
| | GGCGCCCTTTGGACCTTTTGCAATCCTGGAGCAAACAGCAAACACTGTACCAGAATGAACATTGAACTTTGGACTATACCTGAGGGGTGAGGTAAACAACAGGACTATAAATGGCCCGGGAGGCGCCCTTTGGACCTTTTGCAAT |
| | CCTGGCGCACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAACATAGGCAGACCTTAAGGGATGACTCAGTAACA |
| | GATAAGCTTTGTGTGCCTGCAGGGCATATAAAACAGGGGCAAGGCACAGACTCATAGCAGAGCAATCACCACCAAGCCTGGAATAACTGCAGCCACC |
| 386 | >SP0271 (LVR 133 A1) |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTGTACCAGAATGAACATTGAACTTTGGACTATACCTGAGGGGTGA |
| | GGTAAACAACAGGACTATAAATGGCCCGGGAGGCGCCCTTTGGACCTTTTGCAATCCTGGCGCTCTTTTGTTTTACATGAAGGGTCTGGCAGCCAAAGCAATCACTCAAAGTTCAAACCTTATCATTTTTTGCTTTGTTCCTCTT |
| | GGCCTTGGTTTTGTACATCAGCTTTGAAAATACCATCCCAGGGTTAATGCTGGGGTTAATTTATAACTAAGAGTGCTCTAGTTTTGCAATACAGGACATGCTATAAAAATGGAAAGATGTTGCTTTCTGAGAGATGCGCCACC |
| 387 | >SP0272 (LVR_133_V1) |
| | AAGCAAATATTTGTGGTTATGGATTAACTCGAACTGTTTGCCCACTCTATTTGCCCTGTACCAGAATGAACATTGAACTTTGGACTATACCTGAGGGGTGAGGTAAACAACAGGACTATAAATGGCCCGGGAGGCGCCCTTTGGA |
| | CCTTTTGCAATCCTGGCGCTCTTTTGTTTTACATGAAGGGTCTGGCAGCCAAAGCAATCACTCAAAGTTCAAACCTTATCATTTTTTGCTTTGTTCCTCTTGGCCTTGGTTTTGTACATCAGCTTTGAAAATACCATCCCAGGGT |
| | TAATGCTGGGGTTAATTTATAACTAAGAGTGCTCTAGTTTTGCAATACAGGACATGCTATAAAAATGGAAAGATGTTGCTTTCTGAGAGATGCGCCACC |
| 388 | >SP0273 (LVR 133_V2) |
| | GGCGCCCTTTGGACCTTTTGCAATCCTGGAGCAAACAGCAAACACTGTACCAGAATGAACATTGAACTTTGGACTATACCTGAGGGGTGAGGTAAACAACAGGACTATAAATGGCCCGGGAGGCGCCCTTTGGACCTTTTGCAAT |
| | CCTGGCGCTCTTTTGTTTTACATGAAGGGTCTGGCAGCCAAAGCAATCACTCAAAGTTCAAACCTTATCATTTTTTGCTTTGTTCCTCTTGGCCTTGGTTTTGTACATCAGCTTTGAAAATACCATCCCAGGGTTAATGCTGGGG |
| | TTAATTTATAACTAAGAGTGCTCTAGTTTTGCAATACAGGACATGCTATAAAAATGGAAAGATGTTGCTTTCTGAGAGATGCGCCACC |
| 389 | >SP0256 |
| | ATTGGCATCTTCTATTGCTTTTCCTGGTGACTTCATTTTTCACTCTTGGCTAAAAATGGGTCTCTGATGATTTATTCTATCCTGGGTGTTGACAAGCTGAAGAAGTTGTGTGGGGCCTGCTGCCAGTAACCCTGGGTGACGAAGC |
| | GTGACTCACCACTCCGAGGTCAGTGGGGGGATGGAAGGCAGGGGAGTCAGCTGACAAGATCTGCTGCTTTGTCACCAGGCCTTCTGCCAGGCTTTCACTTTCTCGCCAACTTACAAGGCCTTTCTGTGTAAACAATACCTGAACC |
| | TTTACCCCGTTGCCCGGCAACGGCCAGGTCTGTGCCAAGTGTTTGCCGATGACCTAATGATTCTGAGCTTGGCAAAGGTCTTATCTCCCAGCTCGCCCAGGCCCAGTGTTCCAGGAATGTGACCTTTGCTGCAGCAGCCGCTGGA |
| | GGGGGCAGAGGGGATGGGCTGGAGGTTGAGCAAACAGAGCAGCAGAAAAGGCAGTTCCTCTTCTCCAGTGCCCTCCTTCCCTGTCTCTGCCTCTCCCTCCCTTCCTCAGGCATCAGAGCGGAGACTTCAGGGAGACCAGAGCCCA |
| | GCTTGCCAGGCACTGAGCTAGAAGCCCTGCC |
| 390 | >SP0257 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTGGCTGGTTTCTTATAAAACTGATGGAAGATACAAACACTATTA |
| | AAGAACTGTTTGCATGTTGCAAATGATGTCCAAAGTCCAAACATTGTTAATAATTAATACTCCAATAAACATCATGTCAGAATTTCTGTTTTCTTTTCCCTTTGAACCTTTGCAGGATTGCCACATCATCAGGACCACACCTTCA |
| | TCAGGAATGAATATCCGATGACCTAATGATTCTGAGCTTGGCAAAGGTCTTATCTCCCAGCTCGCCCAGGCCCAGTGTTCCAGGAATGTGACCTTTGCTGCAGCAGCCGCTGGAGGGGGCAGAGGGGATGGGCTGGAGGTTGAGC |
| | AAACAGAGCAGCAGAAAAGGCAGTTCCTCTTCTCCAGTGCCCTCCTTCCCTGTCTCTGCCTCTCCCTCCCTTCCTCAGGCATCAGAGCGGAGACTTCAGGGAGACCAGAGCCCAGCTTGCCAGGCACTGAGCTAGAAGCCCTGCC |
| 391 | >SP0258 |
| | CTGTTTGCTGCTTGCAATGTTTGCCCATTTTAGGGAGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAG |
| | GCGCCCTTTGGACCTTTTGCAATCCTGGCGCACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAACATAGGCAGA |
| | CCTTAAGGGATGACTCAGTAACAGATAAGCTTTGTGTGCCTGCAGGGCATATAAAACAGGGGCAAGGCACAGACTCATAGCAGAGCAATCACCACCAAGCCTGGAATAACTGCAGCCACC |
| 392 | >SP0259 |
| | CTGTTTGCTGCTTGCAATGTTTGCCCATTTTAGGGGCTGGTTTCTTATAAAACTGATGGAAGATACAAACACTATTAAAGAACTGTTTGCATGTTGCAAATGATGTCCAAAGTCCAAACATTGTTAATAATTAATACTCCAATAA |
| | ACATCATGTCAGAATTTCTGTTTTCTTTTCCCTTTGAACCTTTGCAGGATTGCCACATCATCAGGACCACACCTTCATCAGGAATGAATATCCGATGACCTAATGATTCTGAGCTTGGCAAAGGTCTTATCTCCCAGCTCGCCCA |
| | GGCCCAGTGTTCCAGGAATGTGACCTTTGCTGCAGCAGCCGCTGGAGGGGGCAGAGGGGATGGGCTGGAGGTTGAGCAAACAGAGCAGCAGAAAAGGCAGTTCCTCTTCTCCAGTGCCCTCCTTCCCTGTCTCTGCCTCTCCCTC |
| | CCTTCCTCAGGCATCAGAGCGGAGACTTCAGGGAGACCAGAGCCCAGCTTGCCAGGCACTGAGCTAGAAGCCCTGCC |
| 393 | >SP0264 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTGCCTTTGCAACAGCTTATCGGAAGCAAACAAGCTGAGGGGAAT |
| | TGAGCAAGAATTTCTGGGATACCAACAGCATAGGAGGAACAAAGGACGTAGAGGGAGGGTTGACTGTCTACACAGGACAAAGCCAATGATTAACCAAACCTCTTGCAGATTTAAATAGGATGGGAACTAGGAGTGGCAGCAATCC |
| | TTTCTTTCAGCTGGAGTGCTCCTCAGGAGCCAGCCCCACCCTTAGAAAAGCCACC |
| 394 | >SP0265 (LVR_SP131_A1) |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTGTACCGGCCCGGGAGGCGCCCTTTGGACCTTTTGCAATCCTGGC |
| | GCACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAACATAGGCAGACCTTAAGGGATGACTCAGTAACAGATAAG |
| | CTTTGTGTGCCTGCAGGGCATATAAAACAGGGGCAAGGCACAGACTCATAGCAGAGCAATCACCACCAAGCCTGGAATAACTGCAGCCACC |
| 395 | >SP0266 (LVR_SP131_V1) |
| | AAGCAAATATTTGTGGTTATGGATTAACTCGAACTGTTTGCCCACTCTATTTGCCCTGTACCGGCCCGGGAGGCGCCCTTTGGACCTTTTGCAATCCTGGCGCACTGAACCCTTGACCCCTGCCCTGCAGCCCCCGCAGCTTGCT |
| | GTTTGCCCACTCTATTTGCCCAGCCCCAGCCCTGGAGAGTCCTTTAGCAGGGCAAAGTGCAACATAGGCAGACCTTAAGGGATGACTCAGTAACAGATAAGCTTTGTGTGCCTGCAGGGCATATAAAACAGGGGCAAGGCACAGA |
| | CTCATAGCAGAGCAATCACCACCAAGCCTGGAATAACTGCAGCCACC |
| 396 | >SP0267 (LVR_SP131_V2) |
| | GGCGCCCTTTGGACCTTTTGCAATCCTGGAGCAAACAGCAAACACTGTACCGGCCCGGGAGGCGCCCTTTGGACCTTTTGCAATCCTGGCGCACTGAACCCTTGACCCCTGCCCTGCAGCCCCCGCAGCTTGCTGTTTGCCCACT |
| | CTATTTGCCCAGCCCCAGCCCTGGAGAGTCCTTTAGCAGGGCAAAGTGCAACATAGGCAGACCTTAAGGGATGACTCAGTAACAGATAAGCTTTGTGTGCCTGCAGGGCATATAAAACAGGGGCAAGGCACAGACTCATAGCAGA |
| | GCAATCACCACCAAGCCTGGAATAACTGCAGCCACC |
| 397 | >SP0268 (LVR 132_A1) |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTGTACCAGAATGAACATTGAACTTTGGACTATACCTGAGGGGTGA |
| | GGTAAACAACAGGACTATAAATGGCCCGGGAGGCGCCCTTTGGACCTTTTGCAATCCTGGCGCACTGAACCCTTGACCCCTGCCCTGCAGCCCCCGCAGCTTGCTGTTTGCCCACTCTATTTGCCCAGCCCCAGCCCTGGAGAGT |
| | CCTTTAGCAGGGCAAAGTGCAACATAGGCAGACCTTAAGGGATGACTCAGTAACAGATAAGCTTTGTGTGCCTGCAGGGCATATAAAACAGGGGCAAGGCACAGACTCATAGCAGAGCAATCACCACCAAGCCTGGAATAACTGC |
| | AGCCACC |
| 398 | >SP0269 (LVR 132 V1) |
| | AAGCAAATATTTGTGGTTATGGATTAACTCGAACTGTTTGCCCACTCTATTTGCCCTGTACCAGAATGAACATTGAACTTTGGACTATACCTGAGGGGTGAGGTAAACAACAGGACTATAAATGGCCCGGGAGGCGCCCTTTGGA |
| | CCTTTTGCAATCCTGGCGCACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAACATAGGCAGACCTTAAGGGATG |
| | ACTCAGTAACAGATAAGCTTTGTGTGCCTGCAGGGCATATAAAACAGGGGCAAGGCACAGACTCATAGCAGAGCAATCACCACCAAGCCTGGAATAACTGCAGCCACC |
| 399 | >SP0270 (LVR 132 V2) |
| | GGCGCCCTTTGGACCTTTTGCAATCCTGGAGCAAACAGCAAACACTGTACCAGAATGAACATTGAACTTTGGACTATACCTGAGGGGTGAGGTAAACAACAGGACTATAAATGGCCCGGGAGGCGCCCTTTGGACCTTTTGCAAT |
| | CCTGGCGCACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAACATAGGCAGACCTTAAGGGATGACTCAGTAACA |
| | GATAAGCTTTGTGTGCCTGCAGGGCATATAAAACAGGGGCAAGGCACAGACTCATAGCAGAGCAATCACCACCAAGCCTGGAATAACTGCAGCCACC |
| 400 | >SP0271 (LVR_133_A1) |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTGTACCAGAATGAACATTGAACTTTGGACTATACCTGAGGGGTGA |
| | GGTAAACAACAGGACTATAAATGGCCCGGGAGGCGCCCTTTGGACCTTTTGCAATCCTGGCGCTCTTTTGTTTTACATGAAGGGTCTGGCAGCCAAAGCAATCACTCAAAGTTCAAACCTTATCATTTTTTGCTTTGTTCCTCTT |
| | GGCCTTGGTTTTGTACATCAGCTTTGAAAATACCATCCCAGGGTTAATGCTGGGGTTAATTTATAACTAAGAGTGCTCTAGTTTTGCAATACAGGACATGCTATAAAAATGGAAAGATGTTGCTTTCTGAGAGATGCGCCACC |
| 401 | >SP0272 (LVR_133_V1) |
| | AAGCAAATATTTGTGGTTATGGATTAACTCGAACTGTTTGCCCACTCTATTTGCCCTGTACCAGAATGAACATTGAACTTTGGACTATACCTGAGGGGTGAGGTAAACAACAGGACTATAAATGGCCCGGGAGGCGCCCTTTGGA |
| | CCTTTTGCAATCCTGGCGCTCTTTTGTTTTACATGAAGGGTCTGGCAGCCAAAGCAATCACTCAAAGTTCAAACCTTATCATTTTTTGCTTTGTTCCTCTTGGCCTTGGTTTTGTACATCAGCTTTGAAAATACCATCCCAGGGT |
| | TAATGCTGGGGTTAATTTATAACTAAGAGTGCTCTAGTTTTGCAATACAGGACATGCTATAAAAATGGAAAGATGTTGCTTTCTGAGAGATGCGCCACC |
| 402 | >SP0273 (LVR 133_V2) |
| | GGCGCCCTTTGGACCTTTTGCAATCCTGGAGCAAACAGCAAACACTGTACCAGAATGAACATTGAACTTTGGACTATACCTGAGGGGTGAGGTAAACAACAGGACTATAAATGGCCCGGGAGGCGCCCTTTGGACCTTTTGCAAT |
| | CCTGGCGCTCTTTTGTTTTACATGAAGGGTCTGGCAGCCAAAGCAATCACTCAAAGTTCAAACCTTATCATTTTTTGCTTTGTTCCTCTTGGCCTTGGTTTTGTACATCAGCTTTGAAAATACCATCCCAGGGTTAATGCTGGGG |
| | TTAATTTATAACTAAGAGTGCTCTAGTTTTGCAATACAGGACATGCTATAAAAATGGAAAGATGTTGCTTTCTGAGAGATGCGCCACC |
| 403 | >SP0368 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCAGTCATATGTTTGCTCACTGAAGGTTACTAGTTAACAGGCATCCC |
| | TTAAACAGGATATAAAAGGACTTCAGCAGGACTGCTCGAAACATCCCACTCAGCCACC |
| 404 | >SP0373 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTGTACCGGCCCGGGAGGCGCCCTTTGGACCTTTTGCAATCCTGGC |
| | GGGGCATATAAAACAGGGGCAAGGCACAGACTCATAGCAGAGCAATCACCACCAAGCCTGGAATAACTGCAGCCACC |
| 405 | >SP0378 |
| | TGCTCTCTGACAAAGATACGGTGGGTCCCACTGATGAACTGTGCTGCCACAGTAAATGTAGCCACTATGCCTATCTCCATTCTGAAGATGTGCCCTGTTCAAACATGTCCTAATACTCTGTCTCTGCAAGGGTCATCAGTAGTTT |
| | TCCATCTTACTCAACATCCTCCCAGTGAGTCATATGTTTGCTCACTGAAGGTTACTAGTTAACAGGCATCCCTTAAACAGGATATAAAAGGACTTCAGCAGGACTGCTCGAAACATCCCACTCAGCCACC |
| 406 | >SP0379 |
| | CCTCCCCGTGTTCCTGCTCTTTGTCCCTCTGTCCTACTTAGACTAATATTTGCCTTGGGTACTGCAAACAGGAAATGGGGGAGGGACAGGAGTAGGGCGGCCCTGTTCAAACATGTCCTAATACTCTGTCTCTGCAAGGGTCATC |
| | AGTAGTTTTCCATCTTACTCAACATCCTCCCAGTGAGTCATATGTTTGCTCACTGAAGGTTACTAGTTAACAGGCATCCCTTAAACAGGATATAAAAGGACTTCAGCAGGACTGCTCGAAACATCCCACTCAGCCACC |
| 407 | >SP0380 |
| | CCGCCCCCACTGAACCCTTGACCCCTGCCCTGCAGCCCCCGCAGCTTGCTGTTTGCCCACTCTATTTGCCCAGCCCCAGCCCTGTTCAAACATGTCCTAATACTCTGTCTCTGCAAGGGTCATCAGTAGTTTTCCATCTTACTCA |
| | ACATCCTCCCAGTGAGTCATATGTTTGCTCACTGAAGGTTACTAGTTAACAGGCATCCCTTAAACAGGATATAAAAGGACTTCAGCAGGACTGCTCGAAACATCCCACTCAGCCACC |
| 408 | >SP0381 |
| | AAGCTTTCTGAACAGCCAAACAGAGATTCCAAAGTTCAGGCACCAAAGTTCAGACCCTAACAGTTATTTACAAGGGTCAGTTAACCCCTGTTCAAACATGTCCTAATACTCTGTCTCTGCAAGGGTCATCAGTAGTTTTCCATCT |
| | TACTCAACATCCTCCCAGTGAGTCATATGTTTGCTCACTGAAGGTTACTAGTTAACAGGCATCCCTTAAACAGGATATAAAAGGACTTCAGCAGGACTGCTCGAAACATCCCACTCAGCCACC |
| 409 | >SP0272 (LVR 133_V1) |
| | AAGCAAATATTTGTGGTTATGGATTAACTCGAACTGTTTGCCCACTCTATTTGCCCTGTACCAGAATGAACATTGAACTTTGGACTATACCTGAGGGGTGAGGTAAACAACAGGACTATAAATGGCCCGGGAGGCGCCCTTTGGA |
| | CCTTTTGCAATCCTGGCGCTCTTTTGTTTTACATGAAGGGTCTGGCAGCCAAAGCAATCACTCAAAGTTCAAACCTTATCATTTTTTGCTTTGTTCCTCTTGGCCTTGGTTTTGTACATCAGCTTTGAAAATACCATCCCAGGGT |
| | TAATGCTGGGGTTAATTTATAACTAAGAGTGCTCTAGTTTTGCAATACAGGACATGCTATAAAAATGGAAAGATGTTGCTTTCTGAGAGATGCGCCACC |
| 410 | >SP0273 (LVR_133_V2) |
| | GGCGCCCTTTGGACCTTTTGCAATCCTGGAGCAAACAGCAAACACTGTACCAGAATGAACATTGAACTTTGGACTATACCTGAGGGGTGAGGTAAACAACAGGACTATAAATGGCCCGGGAGGCGCCCTTTGGACCTTTTGCAAT |
| | CCTGGCGCTCTTTTGTTTTACATGAAGGGTCTGGCAGCCAAAGCAATCACTCAAAGTTCAAACCTTATCATTTTTTGCTTTGTTCCTCTTGGCCTTGGTTTTGTACATCAGCTTTGAAAATACCATCCCAGGGTTAATGCTGGGG |
| | TTAATTTATAACTAAGAGTGCTCTAGTTTTGCAATACAGGACATGCTATAAAAATGGAAAGATGTTGCTTTCTGAGAGATGCGCCACC |
| 411 | >SP0368 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCAGTCATATGTTTGCTCACTGAAGGTTACTAGTTAACAGGCATCCC |
| | TTAAACAGGATATAAAAGGACTTCAGCAGGACTGCTCGAAACATCCCACTCAGCCACC |
| 412 | >SP0373 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTGTACCGGCCCGGGAGGCGCCCTTTGGACCTTTTGCAATCCTGGC |
| | GGGGCATATAAAACAGGGGCAAGGCACAGACTCATAGCAGAGCAATCACCACCAAGCCTGGAATAACTGCAGCCACC |
| 413 | >SP0378 |
| | TGCTCTCTGACAAAGATACGGTGGGTCCCACTGATGAACTGTGCTGCCACAGTAAATGTAGCCACTATGCCTATCTCCATTCTGAAGATGTGCCCTGTTCAAACATGTCCTAATACTCTGTCTCTGCAAGGGTCATCAGTAGTTT |
| | TCCATCTTACTCAACATCCTCCCAGTGAGTCATATGTTTGCTCACTGAAGGTTACTAGTTAACAGGCATCCCTTAAACAGGATATAAAAGGACTTCAGCAGGACTGCTCGAAACATCCCACTCAGCCACC |
| 414 | >SP0379 |
| | CCTCCCCGTGTTCCTGCTCTTTGTCCCTCTGTCCTACTTAGACTAATATTTGCCTTGGGTACTGCAAACAGGAAATGGGGGAGGGACAGGAGTAGGGCGGCCCTGTTCAAACATGTCCTAATACTCTGTCTCTGCAAGGGTCATC |
| | AGTAGTTTTCCATCTTACTCAACATCCTCCCAGTGAGTCATATGTTTGCTCACTGAAGGTTACTAGTTAACAGGCATCCCTTAAACAGGATATAAAAGGACTTCAGCAGGACTGCTCGAAACATCCCACTCAGCCACC |
| 415 | >SP0380 |
| | CCGCCCCCACTGAACCCTTGACCCCTGCCCTGCAGCCCCCGCAGCTTGCTGTTTGCCCACTCTATTTGCCCAGCCCCAGCCCTGTTCAAACATGTCCTAATACTCTGTCTCTGCAAGGGTCATCAGTAGTTTTCCATCTTACTCA |
| | ACATCCTCCCAGTGAGTCATATGTTTGCTCACTGAAGGTTACTAGTTAACAGGCATCCCTTAAACAGGATATAAAAGGACTTCAGCAGGACTGCTCGAAACATCCCACTCAGCCACC |
| 416 | >SP0381 |
| | AAGCTTTCTGAACAGCCAAACAGAGATTCCAAAGTTCAGGCACCAAAGTTCAGACCCTAACAGTTATTTACAAGGGTCAGTTAACCCCTGTTCAAACATGTCCTAATACTCTGTCTCTGCAAGGGTCATCAGTAGTTTTCCATCT |
| | TACTCAACATCCTCCCAGTGAGTCATATGTTTGCTCACTGAAGGTTACTAGTTAACAGGCATCCCTTAAACAGGATATAAAAGGACTTCAGCAGGACTGCTCGAAACATCCCACTCAGCCACC |
| 417 | >SP0384 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGCCGAT |
| | GACCTAATGATTCTGAGCTTGGCAAAGGTCTTATCTCCCAGCTCGCCCAGGCCCAGTGTTCCAGGAATGTGACCTTTGCTGCAGCAGCCGCTGGAGGGGGCAGAGGGGATGGGCTGGAGGTTGAGCAAACAGAGCAGCAGAAAAG |
| | GCAGTTCCTCTTCTCCAGTGCCCTCCTTCCCTGTCTCTGCCTCTCCCTCCCTTCCTCAGGCATCAGAGCGGAGACTTCAGGGAGACCAGAGCCCAGCTTGCCAGGCACTGAGCTAGAAGCCCTGCCGCCACC |
| 418 | >SP0388 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGGCATATAAAACAGGGGCAAGGCACAGACTCATAGCAGAGCAAT |
| | CACCACCAAGCCTGGAATAACTGCAGCCACC |
| 419 | >SP0396 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTGTACCGGCCCGGGAGGCGCCCTTTGGACCTTTTGCAATCCTGGC |
| | GCCCTGGAGAGTCCTTTAGCAGGGCAAAGTGCAACATAGGCAGACCTTAAGGGATGACTCAGTAACAGATAAGCTTTGTGTGCCTGCAGGGCATATAAAACAGGGGCAAGGCACAGACTCATAGCAGAGCAATCACCACCAAGCC |
| | TGGAATAACTGCAGCCACC |
| 420 | >SP0397 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTGTACCCACTGAACCCTTGACCCCTGCCCTGCAGCCCCCGCAGCT |
| | TGCTGTTTGCCCACTCTATTTGCCCAGCCCCAGCCCTGGAGAGTCCTTTAGCAGGGCAAAGTGCAACATAGGCAGACCTTAAGGGATGACTCAGTAACAGATAAGCTTTGTGTGCCTGCAGGGCATATAAAACAGGGGCAAGGCA |
| | CAGACTCATAGCAGAGCAATCACCACCAAGCCTGGAATAACTGCAGCCACC |
| 421 | >SP0398 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTGAGGTTAATTTTTAAAAAGCAGTCAAAAGTCCAAGTGGCCCTT |
| | GGCAGCATTTACTCTCTCTGTTTGCTCTGGTTAATAATCTCAGGAGCACAAACATTCCTGTACCAGAATGAACATTGAACTTTGGACTATACCTGAGGGGTGAGGTAAACAACAGGACTATAAATGGCCCGGGAGGCGCCCTTTG |
| | GACCTTTTGCAATCCTGGCGCACTGAACCCTTGACCCCTGCCCTGCAGCCCCCGCAGCTTGCTGTTTGCCCACTCTATTTGCCCAGCCCCAGCCCTGGAGAGTCCTTTAGCAGGGCAAAGTGCAACATAGGCAGACCTTAAGGGA |
| | TGACTCAGTAACAGATAAGCTTTGTGTGCCTGCAGGGCATATAAAACAGGGGCAAGGCACAGACTCATAGCAGAGCAATCACCACCAAGCCTGGAATAACTGCAGCCACC (SEQ ID NO: 249) |
| 422 | >SP0399 |
| | AGAATGAACATTGAACTTTGGACTATACCTGAGGGGTGAGGTAAACAACAGGACTATAAATGGGCATATAAAACAGGGGCAAGGCACAGACTCATAGCAGAGCAATCACCACCAAGCCTGGAATAACTGCAGCCACC |
| 423 | >SP0403 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTGAAGCAAATATTTGTGGTTATGGATTAACTCGAACTGTTTGCC |
| | CACTCTATTTGCCCTGTACCGGCCCGGGAGGCGCCCTTTGGACCTTTTGCAATCCTGGCGCACTGAACCCTTGACCCCTGCCCTGCAGCCCCCGCAGCTTGCTGTTTGCCCACTCTATTTGCCCAGCCCCAGCCCTGGAGAGTCC |
| | TTTAGCAGGGCAAAGTGCAACATAGGCAGACCTTAAGGGATGACTCAGTAACAGATAAGCTTTGTGTGCCTGCAGGGCATATAAAACAGGGGCAAGGCACAGACTCATAGCAGAGCAATCACCACCAAGCCTGGAATAACTGCAG |
| | CCACC |
| 424 | >SP0404 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTGAAGCAAATATTTGTGGTTATGGATTAACTCGAACTGTTTGCC |
| | CACTCTATTTGCCCTGTACCAGAATGAACATTGAACTTTGGACTATACCTGAGGGGTGAGGTAAACAACAGGACTATAAATGGCCCGGGAGGCGCCCTTTGGACCTTTTGCAATCCTGGCGCACTGAACCCTTGACCCCTGCCCT |
| | GCAGCCCCCGCAGCTTGCTGTTTGCCCACTCTATTTGCCCAGCCCCAGCCCTGGAGAGTCCTTTAGCAGGGCAAAGTGCAACATAGGCAGACCTTAAGGGATGACTCAGTAACAGATAAGCTTTGTGTGCCTGCAGGGCATATAA |
| | AACAGGGGCAAGGCACAGACTCATAGCAGAGCAATCACCACCAAGCCTGGAATAACTGCAGCCACC |
| 425 | >SP0405 |
| | CAGGCTTTCACTTTCTCGCCAACTTACAAGGCCTTTCTGTGTAAACAATACCTGAACCTTTACCCCGTTGCCCGGCAACGGCCAGGTCTGTGCCAAGTGTTTGAAGCAAATATTTGTGGTTATGGATTAACTCGAACTGTTTGCC |
| | CACTCTATTTGCCCTGTACCAGAATGAACATTGAACTTTGGACTATACCTGAGGGGTGAGGTAAACAACAGGACTATAAATGGCCCGGGAGGCGCCCTTTGGACCTTTTGCAATCCTGGCGCACTGAACCCTTGACCCCTGCCCT |
| | GCAGCCCCCGCAGCTTGCTGTTTGCCCACTCTATTTGCCCAGCCCCAGGGGCATATAAAACAGGGGCAAGGCACAGACTCATAGCAGAGCAATCACCACCAAGCCTGGAATAACTGCAGCCACC |
| 426 | >SP0406 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCGGCCCGGGAGGCGCCCTTTGGACCTTTTGCAATCCTGGCGAGTCA |
| | TATGTTTGCTCACTGAAGGTTACTAGTTAACAGGCATCCCTTAAACAGGATATAAAAGGACTTCAGCAGGACTGCTCGAAACATCCCACTCAGCCACC |
| 427 | >SP0407 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCCAGGCTTTCACTTTCTCGCCAACTTACAAGGCCTTTCTGTGTAAA |
| | CAATACCTGAACCTTTACCCCGTTGCCCGGCAACGGCCAGGTCTGTGCCAAGTGTTTGGGGCATATAAAACAGGGGCAAGGCACAGACTCATAGCAGAGCAATCACCACCAAGCCTGGAATAACTGCAGCCACC |
| 428 | >SP0409 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCCCCTGTTCAAACATGTCCTAATACTCTGTCTCTGCAAGGGTCATC |
| | AGTAGTTTTCCATCTTACTCAACATCCTCCCAGTGGGGCATATAAAACAGGGGCAAGGCACAGACTCATAGCAGAGCAATCACCACCAAGCCTGGAATAACTGCAGCCACC |
| 429 | >SP0411 |
| | CCCTGTTCAAACATGTCCTAATACTCTGTCTCTGCAAGGGTCATCAGTAGTTTTCCATCTTACTCAACATCCTCCCAGTGAGTCATATGTTTGCTCACTGAAGGTTACTAGTTAACAGGCATCCCTTAAACAGGATATAAAAGGA |
| | CTTCAGCAGGACTGCTCGAAACATCCCACTCAGCCACC |
| 91 | >SP0412 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCCCCTGTTCAAACATGTCCTAATACTCTGTCTCTGCAAGGGTCATC |
| | AGTAGTTTTCCATCTTACTCAACATCCTCCCAGTGAGTCATATGTTTGCTCACTGAAGGTTACTAGTTAACAGGCATCCCTTAAACAGGATATAAAAGGACTTCAGCAGGACTGCTCGAAACATCCCACTCAGCCACC |
| 430 | >SP0413 |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCTGTACCGGCCCGGGAGGCGCCCTTTGGACCTTTTGCAATCCTGGC |
| | GAGTCATATGTTTGCTCACTGAAGGTTACTAGTTAACAGGCATCCCTTAAACAGGATATAAAAGGACTTCAGCAGGACTGCTCGAAACATCCCACTCAGCCACC |
| 431 | >TTR promoter |
| | TCGAGCTTGGGCTGCAGGTCGAGGGCACTGGGAGGATGTTGAGTAAGATGGAAAACTACTGATGACCCTTGCAGAGACAGAGTATTAGGACATGTTTGAACAGGGGCCGGGCGATCAGCAGGTAGCTCTAGAGGATCCCCGTCTG |
| | TCTGCACATTTCGTAGAGCGAGTGTTCCGATACTCTAATCTCCCTAGGCAAGGTTCATATTTGTGTAGGTTACTTATTCTCCTTTTGTTGACTAAGTCAATAATCAGAATCAGCAGGTTTGGAGTCAGCTTGGCAGGGATCAGCA |
| | GCCTGGGTTGGAAGGAGGGGGTATAAAAGCCCCTTCACCAGGAGAAGCCGTCACACAGA |
| 432 | >LP1 |
| | CCCTAAAATGGGCAAACATTGCAAGCAGCAAACAGCAAACACACAGCCCTCCCTGCCTGCTGACCTTGGAGCTGGGGCAGAGGTCAGAGACCTCTCTGGGCCCATGCCACCTCCAACATCCACTCGACCCCTTGGAATTTCGGTG |
| | GAGAGGAGCAGAGGTTGTCCTGGCGTGGTTTAGGTAGTGTGAGAGGGGAATGACTCCTTTCGGTAAGTGCAGTGGAAGCTGTACACTGCCCAGGCAAAGCGTCCGGGCAGCGTAGGCGGGCGACTCAGATCCCAGCCAGTGGACT |
| | TAGCCCCTGTTTGCTCCTCCGATAACTGGGGTGACCTTGGTTAATATTCACCAGCAGCCTCCCCCGTTGCCCCTCTGGATCCACTGCTTAAATACGGACGAGGACAGGGCCCTGTCTCCTCAGCTTCAGGCACCACCACTGACCT |
| | GGGACAGTGAATCCGGACTCTAAGGTAAATATAAAATTTTTAAGTGTATAATGTGTTAAACTACTGATTCTAATTGTTTCTCTCTTTTAGATTCCAACCTTTGGAACTGAATTCTAGACCACC |
| 433 | >CMV-IE |
| | ATAGTAATCAATTACGGGGTCATTAGTTCATAGCCCATATATGGAGTTCCGCGTTACATAACTTACGGTAAATGGCCCGCCTGGCTGACCGCCCAACGACCCCCGCCCATTGACGTCAATAATGACGTATGTTCCCATAGTAACG |
| | CCAATAGGGACTTTCCATTGACGTCAATGGGTGGAGTATTTACGGTAAACTGCCCACTTGGCAGTACATCAAGTGTATCATATGCCAAGTACGCCCCCTATTGACGTCAATGACGGTAAATGGCCCGCCTGGCATTATGCCCAGT |
| | ACATGACCTTATGGGACTTTCCTACTTGGCAGTACATCTACGTATTAGTCATCGCTATTACCATGGTGATGCGGTTTTGGCAGTACATCAATGGGCGTGGATAGCGGTTTGACTCACGGGGATTTCCAAGTCTCCACCCCATTGA |
| | CGTCAATGGGAGTTTGTTTTGGCACCAAAATCAACGGGACTTTCCAAAATGTCGTAACAACTCCGCCCCATTGACGCAAATGGGCGGTAGGCGTGTACGGTGGGAGGTCTATATAAGCAGAGCTGGTTTAGTGAACCGTCAGATC |
| 434 | >CBA |
| | AGATCTGAATTCGGTACCTAGTTATTAATAGTAATCAATTACGGGGTCATTAGTTCATAGCCCATATATGGAGTTCCGCGTTACATAACTTACGGTAAATGGCCCGCCTGGCTGACCGCCCAACGACCCCCGCCCATTGACGTCA |
| | ATAATGACGTATGTTCCCATAGTAACGCCAATAGGGACTTTCCATTGACGTCAATGGGTGGAGTATTTACGGTAAACTGCCCACTTGGCAGTACATCAAGTGTATCATATGCCAAGTACGCCCCCTATTGACGTCAATGACGGTA |
| | AATGGCCCGCCTGGCATTATGCCCAGTACATGACCTTATGGGACTTTCCTACTTGGCAGTACATCTACGTATTAGTCATCGCTATTACCATGGTCGAGGTGAGCCCCACGTTCTGCTTCACTCTCCCCATCTCCCCCCCCTCCCC |
| | ACCCCCAATTTTGTATTTATTTATTTTTTAATTATTTTGTGCAGCGATGGGGGCGGGGGGGGGGGGGGGGCGCGCGCCAGGCGGGGCGGGGCGGGGCGAGGGGCGGGGCGGGGCGAGGCGGAGAGGTGCGGCGGCAGCCAATCAG |
| | AGCGGCGCGCTCCGAAAGTTTCCTTTTATGGCGAGGCGGCGGCGGCGGCGGCCCTATAAAAAGCGAAGCGCGCGGCGGGCGGGAGTCGCTGCGCGCTGCCTTCGCCCCGTGCCCCGCTCCGCCGCCGCCTCGCGCCGCCCGCCCC |
| | GGCTCTGACTGACCGCGTTACTCCCACAGGTGAGCGGGCGGGACGGCCCTTCTCCTCCGGGCTGTAATTAGCGCTTGGTTTAATGACGGCTTGTTTCTTTTCTGTGGCTGCGTGAAAGCCTTGAGGGGCTCCGGGAGGGCCCTTT |
| | GTGCGGGGGGAGCGGCTCGGGGGGTGCGTGCGTGTGTGTGTGCGTGGGGAGCGCCGCGTGCGGCTCCGCGCTGCCCGGCGGCTGTGAGCGCTGCGGGCGCGGCGCGGGGCTTTGTGCGCTCCGCAGTGTGCGCGAGGGGAGCGCG |
| | GCCGGGGGCGGTGCCCCGCGGTGCGGGGGGGGCTGCGAGGGGAACAAAGGCTGCGTGCGGGGTGTGTGCGTGGGGGGGTGAGCAGGGGGTGTGGGCGCGTCGGTCGGGCTGCAACCCCCCCTGCACCCCCCTCCCCGAGTTGCTG |
| | AGCACGGCCCGGCTTCGGGTGCGGGGCTCCGTACGGGGCGTGGCGCGGGGCTCGCCGTGCCGGGCGGGGGGTGGCGGCAGGTGGGGGTGCCGGGCGGGGCGGGGCCGCCTCGGGCCGGGGAGGGCTCGGGGGAGGGGCGCGGCGG |
| | CCCCCGGAGCGCCGGCGGCTGTCGAGGCGCGGCGAGCCGCAGCCATTGCCTTTTATGGTAATCGTGCGAGAGGGCGCAGGGACTTCCTTTGTCCCAAATCTGTGCGGAGCCGAAATCTGGGAGGCGCCGCCGCACCCCCTCTAGC |
| | GGGCGCGGGGCGAAGCGGTGCGGCGCCGGCAGGAAGGAAATGGGCGGGGAGGGCCTTCGTGCGTCGCCGCGCCGCCGTCCCCTTCTCCCTCTCCAGCCTCGGGGCTGTCCGCGGGGGGACGGCTGCCTTCGGGGGGGACGGGGCA |
| | GGGCGGGGTTCGGCTTCTGGCGTGTGACCGGCGGCTCTAGAGCCTCTGCTAACCATGTTCATGCCTTCTTCTTTTTCCTACAGCTCCTGGGCAACGTGCTGGTTATTGTGCTGTCTCATCATTTTGGCAAAGAATTCCTCGAAGA |
| | TCTAGGCAACGCGTCTCGAGGCGGCCGCCGCCACC |
| 435 | >TBG |
| | AGGTTAATTTTTAAAAAGCAGTCAAAAGTCCAAGTGGCCCTTGGCAGCATTTACTCTCTCTGTTTGCTCTGGTTAATAATCTCAGGAGCACAAACATTCCAGATCCAGGTTAATTTTTAAAAAGCAGTCAAAAGTCCAAGTGGCC |
| | CTTGGCAGCATTTACTCTCTCTGTTTGCTCTGGTTAATAATCTCAGGAGCACAAACATTCCAGATCCGGCGCGCCAGGGCTGGAAGCTACCTTTGACATCATTTCCTCTGCGAATGCATGTATAATTTCTACAGAACCTATTAGA |
| | AAGGATCACCCAGCCTCTGCTTTTGTACAACTTTCCCTTAAAAAACTGCCAATTCCACTGCTGTTTGGCCCAATAGTGAGAACTTTTTCCTGCTGCCTCTTGGTGCTTTTGCCTATGGCCCCTATTCTGCCTGCTGAAGACACTC |
| | TTGCCAGCATGGACTTAAACCCCTCCAGCTCTGACAATCCTCTTTCTCTTTTGTTTTACATGAAGGGTCTGGCAGCCAAAGCAATCACTCAAAGTTCAAACCTTATCATTTTTTGCTTTGTTCCTCTTGGCCTTGGTTTTGTACA |
| | TCAGCTTTGAAAATACCATCCCAGGGTTAATGCTGGGGTTAATTTATAACTAAGAGTGCTCTAGTTTTGCAATACAGGACATGCTATAAAAATGGAAAGATGTTGCTTTCTGAGAGACTGCAGAAGTTGGTCGTGAGGCACTGGG |
| | CAGGTAAGTATCAAGGTTACAAGACAGGTTTAAGGAGACCAATAGAAACTGGGCTTGTCGAGACAGAGAAGACTCTTGCGTTTCTGATAGGCACCTATTGGTCTTACTGACATCCACTTTGCCTTTCTCTCCACAGGGCAATCCG |
| | GTACTGTTGGTAAAGCCACC |
| 438 | >CMV-IE 5′ UTR with 3′ Kozac GCCACC. |
| | TCAGATCGCCTGGAGACGCCATCCACGCTGTTTTGACCTCCATAGAAGACACCGGGACCGATCCAGCCTCCGCGGCCGGGAACGGTGCATTGGAACGCGGATTCCCCGTGCCAAGAGTGACGTAAGTACCGCCTATAGACTCTAT |
| | AGGCACACCCCTTTGGCTCTTATGCATGAACGGTGGAGGGCAGTGTAGTCTGAGCAGTACTCGTTGCTGCCGCGCGCGCCACCAGACATAATAGCTGACAGACTAACAGACTGTTCCTTTCCATGGGTCTTTTCTGCAGGCCACC |
| 439 | >LVR 0243-5′ UTR |
| | TGA AATGCCTGCC ATATATTAGT GCCCTGAAGT CCAAAGGTAG AGGAACCGAG TGTTTAAAAA TTACTGTGGC TGTGGAGTCA ACATGATGTA AAAAAACAAA CATTTGGATA ACACCAAGAA |
| | GCCAGATATG GTTGAAATGT TGACTGGTTG ACAAAAATAA TTTGGGTTGC TTAATGGTGC ACAAAGGTAA TGCAAAAAGG TTAATTTTTA AAAAGCAGTC AAAAGTCCAA GTGGCCCTTG GCAGCATTTA |
| | CTCTCTCTGT TTGCTCTGGT TAATAATCTC AGGAGCACAA ACATTCCGGC CCGGGAGGCG CCCTTTGGAC CTTTTGCAAT CCTGGCGCAC TGAACCCTTG ACCCCTGCCC TGCAGCCCCC GCAGCTTGCT |
| | GTTTGCCCAC TCTATTTGCC CAGCCCCAGC CCTGGAGAGT CCTTTAGCAG GGCAAAGTGC AACATAGGCA GACCTTAAGG GATGACTCAG TAACAGATAA GCTTTGTGTG CCTGCAGGGC ATATAAAACA |
| | GGGGCAAGGC ACAGACTCAT AGCAGAGCAA T |
| 440 | >LVR 0412-5′ UTR |
| | AGG TTAATTTTTA AAAAGCAGTC AAAAGTCCAA GTGGCCCTTG GCAGCATTTA CTCTCTCTGT TTGCTCTGGT TAATAATCTC AGGAGCACAA ACATTCCCCC TGTTCAAACA TGTCCTAATA |
| | CTCTGTCTCT GCAAGGGTCA TCAGTAGTTT TCCATCTTAC TCAACATCCT CCCAGTGAGT CATATGTTTG CTCACTGAAG GTTACTAGTT AACAGGCATC CCTTAAACAG GATATAAAAG GACTTCAGCA |
| | GGACTGCTCG AAACATCCCA CT |
| 441 | >Al promoter (LVR_SP0131_A1) -5′ UTRs |
| | AGG TTAATTTTTA AAAAGCAGTC AAAAGTCCAA GTGGCCCTTG GCAGCATTTA CTCTCTCTGT TTGCTCTGGT TAATAATCTC AGGAGCACAA ACATTCCTGT ACCGGCCCGG GAGGCGCCCT |
| | TTGGACCTTT TGCAATCCTG GCGCACTGAA CCCTTGACCC CTGCCCTGCA GCCCCCGCAG CTTGCTGTTT GCCCACTCTA TTTGCCCAGC CCCAGCCCTG GAGAGTCCTT TAGCAGGGCA AAGTGCAACA |
| | TAGGCAGACC TTAAGGGATG ACTCAGTAAC AGATAAGCTT TGTGTGCCTG CAGGGCATAT AAAACAGGGG CAAGGCACAG ACTCATAGCA GAGCAAT |
| 442 | >MVM Intron |
| | CTAGCCCTAAGGTAAGTTGGCGCCGTTTAAGGGATGGTTGGTTGGTGGGGTATTAATGTTTAATTACCTTTTTTACAGGCCTGAA |
| 443 | >HBB2 intron sequence |
| | GTACACATAT TGACCAAATC AGGGTAATTT TGCATTTGTA ATTTTAAAAA ATGCTTTCTT CTTTTAATAT ACTTTTTTGT TTATCTTATT TCTAATACTT TCCCTAATCT CTTTCTTTCA GGGCAATAAT |
| | GATACAATGT ATCATGCCTC TTTGCACCAT TCTAAAGAAT AACAGTGATA ATTTCTGGGT TAAGGCAATA GCAATATTTC TGCATATAAA TATTTCTGCA TATAAATTGT AACTGATGTA AGAGGTTTCA |
| | TATTGCTAAT AGCAGCTACA ATCCAGCTAC CATTCTGCTT TTATTTTCTG GTTGGGATAA GGCTGGATTA TTCTGAGTCC AAGCTAGGCC CTTTTGCTAA TCTTGTTCAT ACCTCTTATC TTCCTCCCAC |
| | AGCTCCTGGG CAACCTGCTG GTCTCTCTGC TGGCCCATCA CTTTGGCAAA |
| | G |
| 444 | >HBB INTRON |
| | TACTAGCAGCTACAATCCAGCTACCATTCTGCTTTTATTTTATGGTTGGGATAAGGCTGGATTATTCTGAGTCCAAGCTAGGCCCTTTTGCTAATCATGTTCATACCTCTTATCTTCCTCCCACAGCTCCTGGGCAACGTGCTGG |
| | TCTGTGTGCTGGCCCATCACTTTGGCAAAGAATT |
| 445 | >UBC INTRON |
| | ACTTGGTGAGTAGCGGGCTGCTGGGCTGGCCGGGGCTTTCGTGGCCGCCGGGCCGCTCGGTGGGACGGAAGCGTGTGGAGAGACCGCCAAGGGCTGTAGTCTGGGTCCGCGAGCAAGGTTGCCCTGAACTGGGGGTTGGGGGGAG |
| | CGCAGCAAAATGGCGGCTGTTCCCGAGTCTTGAATGGAAGACGCTTGTGAGGCGGGCTGTGAGGTCGTTGAAACAAGGTGGGGGGCATGGTGGGCGGCAAGAACCCAAGGTCTTGAGGCCTTCGCTAATGCGGGAAAGCTCTTAT |
| | TCGGGTGAGATGGGCTGGGGCACCATCTGGGGACCCTGACGTGAAGTTTGTCACTGACTGGAGAACTCGGTTTGTCGTCTGTTGCGGGGGCGGCAGTTATGCGGTGCCGTTGGGCAGTGCACCCGTACCTTTGGGAGCGCGCGCC |
| | CTCGTCGTGTCGTGACGTCACCCGTTCTGTTGGCTTATAATGCAGGGTGGGGCCACCTGCCGGTAGGTGTGCGGTAGGCTTTTCTCCGTCGCAGGACGCAGGGTTCGGGCCTAGGGTAGGCTCTCCTGAATCGACAGGCGCCGGA |
| | CCTCTGGTGAGGGGAGGGATAAGTGAGGCGTCAGTTTCTTTGGTCGGTTTTATGTACCTATCTTCTTAAGTAGCTGAAGCTCCGGTTTTGAACTATGCGCTCGGGGTTGGCGAGTGTGTTTTGTGAAGTTTTTTAGGCACCTTTT |
| | GAAATGTAATCATTTGGGTCAATATGTAATTTTCAGTGTTAGACTAGTAAATTGTCCGCTAAATTCTGGCCGTTTTTGGCTTTTTTGTTAGAC |
| 446 | >MODIFIED CMVIE INTRON SEQUENCE. |
| | CAAGAGTGACGTAAGTACCGCCTATAGACTCTATAGGCACACCCCTTTGGCTCTTATGCATGAACGGTGGAGGGCAGTGTAGTCTGAGCAGTACTCGTTGCTGCCGCGCGCGCCACCAGACATAATAGCTGACAGACTAACAGAC |
| | TGTTCCTTTCCATGGGTCTTTTCTGCAG |
| 465 | >termination sequence |
| | TGATAACTCGAG |
| 464 | |
| 463 | >AAV3B CAPSID NUCLEOTIDE SEQUENCE |
| | ATGGCTGCTGACGGTTATCTTCCAGATTGGCTCGAGGACAACCTTTCTGAAGGCATTCGTGAGTGGTGGGCTCTGAAACCTGGAGTCCCTCAACCCAAAGCGAACCAACAACACCAGGACAACCGTCGGGGTCTTGTGCTTCCGG |
| | GTTACAAATACCTCGGACCCGGTAACGGACTCGACAAAGGAGAGCCGGTCAACGAGGCGGACGCGGCAGCCCTCGAACACGACAAAGCTTACGACCAGCAGCTCAAGGCCGGTGACAACCCGTACCTCAAGTACAACCACGCCGA |
| | CGCCGAGTTTCAGGAGCGTCTTCAAGAAGATACGTCTTTTGGGGGCAACCTTGGCAGAGCAGTCTTCCAGGCCAAAAAGAGGATCCTTGAGCCTCTTGGTCTGGTTGAGGAAGCAGCTAAAACGGCTCCTGGAAAGAAGAGGCCT |
| | GTAGATCAGTCTCCTCAGGAACCGGACTCATCATCTGGTGTTGGCAAATCGGGCAAACAGCCTGCCAGAAAAAGACTAAATTTCGGTCAGACTGGCGACTCAGAGTCAGTCCCAGACCCTCAACCTCTCGGAGAACCACCAGCAG |
| | CCCCCACAAGTTTGGGATCTAATACAATGGCTTCAGGCGGTGGCGCACCAATGGCAGACAATAACGAGGGTGCCGATGGAGTGGGTAATTCCTCAGGAAATTGGCATTGCGATTCCCAATGGCTGGGCGACAGAGTCATCACCAC |
| | CAGCACCAGAACCTGGGCCCTGCCCACTTACAACAACCATCTCTACAAGCAAATCTCCAGCCAATCAGGAGCTTCAAACGACAACCACTACTTTGGCTACAGCACCCCTTGGGGGTATTTTGACTTTAACAGATTCCACTGCCAC |
| | TTCTCACCACGTGACTGGCAGCGACTCATTAACAACAACTGGGGATTCCGGCCCAAGAAACTCAGCTTCAAGCTCTTCAACATCCAAGTTAAAGAGGTCACGCAGAACGATGGCACGACGACTATTGCCAATAACCTTACCAGCA |
| | CGGTTCAAGTGTTTACGGACTCGGAGTATCAGCTCCCGTACGTGCTCGGGTCGGCGCACCAAGGCTGTCTCCCGCCGTTTCCAGCGGACGTCTTCATGGTCCCTCAGTATGGATACCTCACCCTGAACAACGGAAGTCAAGCGGT |
| | GGGACGCTCATCCTTTTACTGCCTGGAGTACTTCCCTTCGCAGATGCTAAGGACTGGAAATAACTTCCAATTCAGCTATACCTTCGAGGATGTACCTTTTCACAGCAGCTACGCTCACAGCCAGAGTTTGGATCGCTTGATGAAT |
| | CCTCTTATTGATCAGTATCTGTACTACCTGAACAGAACGCAAGGAACAACCTCTGGAACAACCAACCAATCACGGCTGCTTTTTAGCCAGGCTGGGCCTCAGTCTATGTCTTTGCAGGCCAGAAATTGGCTACCTGGGCCCTGCT |
| | ACCGGCAACAGAGACTTTCAAAGACTGCTAACGACAACAACAACAGTAACTTTCCTTGGACAGCGGCCAGCAAATATCATCTCAATGGCCGCGACTCGCTGGTGAATCCAGGACCAGCTATGGCCAGTCACAAGGACGATGAAGA |
| | AAAATTTTTCCCTATGCACGGCAATCTAATATTTGGCAAAGAAGGGACAACGGCAAGTAACGCAGAATTAGATAATGTAATGATTACGGATGAAGAAGAGATTCGTACCACCAATCCTGTGGCAACAGAGCAGTATGGAACTGTG |
| | GCAAATAACTTGCAGAGCTCAAATACAGCTCCCACGACTAGAACTGTCAATGATCAGGGGGCCTTACCTGGCATGGTGTGGCAAGATCGTGACGTGTACCTTCAAGGACCTATCTGGGCAAAGATTCCTCACACGGATGGACACT |
| | TTCATCCTTCTCCTCTGATGGGAGGCTTTGGACTGAAACATCCGCCTCCTCAAATCATGATCAAAAATACTCCGGTACCGGCAAATCCTCCGACGACTTTCAGCCCGGCCAAGTTTGCTTCATTTATCACTCAGTACTCCACTGG |
| | ACAGGTCAGCGTGGAAATTGAGTGGGAGCTACAGAAAGAAAACAGCAAACGTTGGAATCCAGAGATTCAGTACACTTCCAACTACAACAAGTCTGTTAATGTGGACTTTACTGTAGACACTAATGGTGTTTATAGTGAACCTCGC |
| | CCTATTGGAACCCGGTATCTCACACGAAACTTG |
| 452 | >AAV3B CAPSID AMINO ACID SEQUENCE |
| | MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRP |
| | VDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDENRFHCH |
| | FSPRDWQRLINNNWGFRPKKLSFKLFNIQVKEVTQNDGTTTIANNLTSTVQVFTDSEYQLPYVLGSAHQGCLPPFPADVEMVPQYGYLTLNNGSQAVGRSSFYCLEYFPSQMLRTGNNFQFSYTFEDVPFHSSYAHSQSLDRLMN |
| | PLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTV |
| | ANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDINGVYSEPR |
| | PIGTRYLTRNL |
| 453 | >AAV3B 265D CAPSID NUCLEOTIDE SEQUENCE |
| | ATGGCTGCTGACGGTTATCTTCCAGATTGGCTCGAGGACAACCTTTCTGAAGGCATTCGTGAGTGGTGGGCTCTGAAACCTGGAGTCCCTCAACCCAAAGCGAACCAACAACACCAGGACAACCGTCGGGGTCTTGTGCTTCCGG |
| | GTTACAAATACCTCGGACCCGGTAACGGACTCGACAAAGGAGAGCCGGTCAACGAGGCGGACGCGGCAGCCCTCGAACACGACAAAGCTTACGACCAGCAGCTCAAGGCCGGTGACAACCCGTACCTCAAGTACAACCACGCCGA |
| | CGCCGAGTTTCAGGAGCGTCTTCAAGAAGATACGTCTTTTGGGGGCAACCTTGGCAGAGCAGTCTTCCAGGCCAAAAAGAGGATCCTTGAGCCTCTTGGTCTGGTTGAGGAAGCAGCTAAAACGGCTCCTGGAAAGAAGAGGCCT |
| | GTAGATCAGTCTCCTCAGGAACCGGACTCATCATCTGGTGTTGGCAAATCGGGCAAACAGCCTGCCAGAAAAAGACTAAATTTCGGTCAGACTGGCGACTCAGAGTCAGTCCCAGACCCTCAACCTCTCGGAGAACCACCAGCAG |
| | CCCCCACAAGTTTGGGATCTAATACAATGGCTTCAGGCGGTGGCGCACCAATGGCAGACAATAACGAGGGTGCCGATGGAGTGGGTAATTCCTCAGGAAATTGGCATTGCGATTCCCAATGGCTGGGCGACAGAGTCATCACCAC |
| | CAGCACCAGAACCTGGGCCCTGCCCACTTACAACAACCATCTCTACAAGCAAATCTCCAGCCAATCAGATGCTTCAAACGACAACCACTACTTTGGCTACAGCACCCCTTGGGGGTATTTTGACTTTAACAGATTCCACTGCCAC |
| | TTCTCACCACGTGACTGGCAGCGACTCATTAACAACAACTGGGGATTCCGGCCCAAGAAACTCAGCTTCAAGCTCTTCAACATCCAAGTTAAAGAGGTCACGCAGAACGATGGCACGACGACTATTGCCAATAACCTTACCAGCA |
| | CGGTTCAAGTGTTTACGGACTCGGAGTATCAGCTCCCGTACGTGCTCGGGTCGGCGCACCAAGGCTGTCTCCCGCCGTTTCCAGCGGACGTCTTCATGGTCCCTCAGTATGGATACCTCACCCTGAACAACGGAAGTCAAGCGGT |
| | GGGACGCTCATCCTTTTACTGCCTGGAGTACTTCCCTTCGCAGATGCTAAGGACTGGAAATAACTTCCAATTCAGCTATACCTTCGAGGATGTACCTTTTCACAGCAGCTACGCTCACAGCCAGAGTTTGGATCGCTTGATGAAT |
| | CCTCTTATTGATCAGTATCTGTACTACCTGAACAGAACGCAAGGAACAACCTCTGGAACAACCAACCAATCACGGCTGCTTTTTAGCCAGGCTGGGCCTCAGTCTATGTCTTTGCAGGCCAGAAATTGGCTACCTGGGCCCTGCT |
| | ACCGGCAACAGAGACTTTCAAAGACTGCTAACGACAACAACAACAGTAACTTTCCTTGGACAGCGGCCAGCAAATATCATCTCAATGGCCGCGACTCGCTGGTGAATCCAGGACCAGCTATGGCCAGTCACAAGGACGATGAAGA |
| | AAAATTTTTCCCTATGCACGGCAATCTAATATTTGGCAAAGAAGGGACAACGGCAAGTAACGCAGAATTAGATAATGTAATGATTACGGATGAAGAAGAGATTCGTACCACCAATCCTGTGGCAACAGAGCAGTATGGAACTGTG |
| | GCAAATAACTTGCAGAGCTCAAATACAGCTCCCACGACTAGAACTGTCAATGATCAGGGGGCCTTACCTGGCATGGTGTGGCAAGATCGTGACGTGTACCTTCAAGGACCTATCTGGGCAAAGATTCCTCACACGGATGGACACT |
| | TTCATCCTTCTCCTCTGATGGGAGGCTTTGGACTGAAACATCCGCCTCCTCAAATCATGATCAAAAATACTCCGGTACCGGCAAATCCTCCGACGACTTTCAGCCCGGCCAAGTTTGCTTCATTTATCACTCAGTACTCCACTGG |
| | ACAGGTCAGCGTGGAAATTGAGTGGGAGCTACAGAAAGAAAACAGCAAACGTTGGAATCCAGAGATTCAGTACACTTCCAACTACAACAAGTCTGTTAATGTGGACTTTACTGTAGACACTAATGGTGTTTATAGTGAACCTCGC |
| | CCTATTGGAACCCGGTATCTCACACGAAACTTG |
| 454 | >AAV3B 265D CAPSID AMINO ACID |
| | MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRP |
| | VDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSDASNDNHYFGYSTPWGYFDENRFHCH |
| | FSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMN |
| | PLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTV |
| | ANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDINGVYSEPR |
| | PIGTRYLTRNL |
| 455 | >AAV3B ST (S663V+T492V) CAPSID NUCLEOTIDE SEQUENCE |
| | ATGGCTGCTGACGGTTATCTTCCAGATTGGCTCGAGGACAACCTTTCTGAAGGCATTCGTGAGTGGTGGGCTCTGAAACCTGGAGTCCCTCAACCCAAAGCGAACCAACAACACCAGGACAACCGTCGGGGTCTTGTGCTTCCGG |
| | GTTACAAATACCTCGGACCCGGTAACGGACTCGACAAAGGAGAGCCGGTCAACGAGGCGGACGCGGCAGCCCTCGAACACGACAAAGCTTACGACCAGCAGCTCAAGGCCGGTGACAACCCGTACCTCAAGTACAACCACGCCGA |
| | CGCCGAGTTTCAGGAGCGTCTTCAAGAAGATACGTCTTTTGGGGGCAACCTTGGCAGAGCAGTCTTCCAGGCCAAAAAGAGGATCCTTGAGCCTCTTGGTCTGGTTGAGGAAGCAGCTAAAACGGCTCCTGGAAAGAAGAGGCCT |
| | GTAGATCAGTCTCCTCAGGAACCGGACTCATCATCTGGTGTTGGCAAATCGGGCAAACAGCCTGCCAGAAAAAGACTAAATTTCGGTCAGACTGGCGACTCAGAGTCAGTCCCAGACCCTCAACCTCTCGGAGAACCACCAGCAG |
| | CCCCCACAAGTTTGGGATCTAATACAATGGCTTCAGGCGGTGGCGCACCAATGGCAGACAATAACGAGGGTGCCGATGGAGTGGGTAATTCCTCAGGAAATTGGCATTGCGATTCCCAATGGCTGGGCGACAGAGTCATCACCAC |
| | CAGCACCAGAACCTGGGCCCTGCCCACTTACAACAACCATCTCTACAAGCAAATCTCCAGCCAATCAGATGCTTCAAACGACAACCACTACTTTGGCTACAGCACCCCTTGGGGGTATTTTGACTTTAACAGATTCCACTGCCAC |
| | TTCTCACCACGTGACTGGCAGCGACTCATTAACAACAACTGGGGATTCCGGCCCAAGAAACTCAGCTTCAAGCTCTTCAACATCCAAGTTAAAGAGGTCACGCAGAACGATGGCACGACGACTATTGCCAATAACCTTACCAGCA |
| | CGGTTCAAGTGTTTACGGACTCGGAGTATCAGCTCCCGTACGTGCTCGGGTCGGCGCACCAAGGCTGTCTCCCGCCGTTTCCAGCGGACGTCTTCATGGTCCCTCAGTATGGATACCTCACCCTGAACAACGGAAGTCAAGCGGT |
| | GGGACGCTCATCCTTTTACTGCCTGGAGTACTTCCCTTCGCAGATGCTAAGGACTGGAAATAACTTCCAATTCAGCTATACCTTCGAGGATGTACCTTTTCACAGCAGCTACGCTCACAGCCAGAGTTTGGATCGCTTGATGAAT |
| | CCTCTTATTGATCAGTATCTGTACTACCTGAACAGAACGCAAGGAACAACCTCTGGAACAACCAACCAATCACGGCTGCTTTTTAGCCAGGCTGGGCCTCAGTCTATGTCTTTGCAGGCCAGAAATTGGCTACCTGGGCCCTGCT |
| | ACCGGCAACAGAGACTTTCAAAGGTAGCTAACGACAACAACAACAGTAACTTTCCTTGGACAGCGGCCAGCAAATATCATCTCAATGGCCGCGACTCGCTGGTGAATCCAGGACCAGCTATGGCCAGTCACAAGGACGATGAAGA |
| | AAAATTTTTCCCTATGCACGGCAATCTAATATTTGGCAAAGAAGGGACAACGGCAAGTAACGCAGAATTAGATAATGTAATGATTACGGATGAAGAAGAGATTCGTACCACCAATCCTGTGGCAACAGAGCAGTATGGAACTGTG |
| | GCAAATAACTTGCAGAGCTCAAATACAGCTCCCACGACTAGAACTGTCAATGATCAGGGGGCCTTACCTGGCATGGTGTGGCAAGATCGTGACGTGTACCTTCAAGGACCTATCTGGGCAAAGATTCCTCACACGGATGGACACT |
| | TTCATCCTTCTCCTCTGATGGGAGGCTTTGGACTGAAACATCCGCCTCCTCAAATCATGATCAAAAATACTCCGGTACCGGCAAATCCTCCGACGACTTTCGTACCGGCCAAGTTTGCTTCATTTATCACTCAGTACTCCACTGG |
| | ACAGGTCAGCGTGGAAATTGAGTGGGAGCTACAGAAAGAAAACAGCAAACGTTGGAATCCAGAGATTCAGTACACTTCCAACTACAACAAGTCTGTTAATGTGGACTTTACTGTAGACACTAATGGTGTTTATAGTGAACCTCGC |
| | CCTATTGGAACCCGGTATCTCACACGAAACTTG |
| 456 | >AAV3B ST (S663V+T492V) AMINO ACID SEQUENCE |
| | MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSEGGNLGRAVFQAKKRILEPLGLVEEAAKTAPGKKRP |
| | VDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDENRFHCH |
| | FSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMN |
| | PLIDQYLYYLNRTQGTTSGTTNQSRLLFSQAGPQSMSLQARNWLPGPCYRQQRLSKVANDNNNSNFPWTAASKYHLNGRDSLVNPGPAMASHKDDEEKFFPMHGNLIFGKEGTTASNAELDNVMITDEEEIRTTNPVATEQYGTV |
| | ANNLQSSNTAPTTRTVNDQGALPGMVWQDRDVYLQGPIWAKIPHTDGHFHPSPLMGGFGLKHPPPQIMIKNTPVPANPPTTFVPAKFASFITQYSTGQVSVEIEWELQKENSKRWNPEIQYTSNYNKSVNVDFTVDINGVYSEPR |
| | PIGTRYLTRNL |
| 457 | >AAV3B265D549A CAPSID NUCLEOTIDE SEQUENCE |
| | GAAACCTGGAGTCCCTCAACCCAAAGCGAACCAACAACACCAGGACAACCGTCGGGGTCTTGTGCTTCCGGGTTACAAATACCTCGGACCCGGTAACGGACTCGACAAAGGAGAGCCGGTCAACGAGGCGGACGCGGCAGCCCTC |
| | GAACACGACAAAGCTTACGACCAGCAGCTCAAGGCCGGTGACAACCCGTACCTCAAGTACAACCACGCCGACGCCGAGTTTCAGGAGCGTCTTCAAGAAGATACGTCTTTTGGGGGCAACCTTGGCAGAGCAGTCTTCCAGGCCA |
| | AAAAGAGGATCCTTGAGCCTCTTGGTCTGGTTGAGGAAGCAGCTAAAACGGCTCCTGGAAAGAAGAGGCCTGTAGATCAGTCTCCTCAGGAACCGGACTCATCATCTGGTGTTGGCAAATCGGGCAAACAGCCTGCCAGAAAAAG |
| | ACTAAATTTCGGTCAGACTGGCGACTCAGAGTCAGTCCCAGACCCTCAACCTCTCGGAGAACCACCAGCAGCCCCCACAAGTTTGGGATCTAATACAATGGCTTCAGGCGGTGGCGCACCAATGGCAGACAATAACGAGGGTGCC |
| | GATGGAGTGGGTAATTCCTCAGGAAATTGGCATTGCGATTCCCAATGGCTGGGCGACAGAGTCATCACCACCAGCACCAGAACCTGGGCCCTGCCCACTTACAACAACCATCTCTACAAGCAAATCTCCAGCCAATCAGGAGCTT |
| | CAAACGACAACCACTACTTTGGCTACAGCACCCCTTGGGGGTATTTTGACTTTAACAGATTCCACTGCCACTTCTCACCACGTGACTGGCAGCGACTCATTAACAACAACTGGGGATTCCGGCCCAAGAAACTCAGCTTCAAGCT |
| | CTTCAACATCCAAGTTAAAGAGGTCACGCAGAACGATGGCACGACGACTATTGCCAATAACCTTACCAGCACGGTTCAAGTGITTACGGACTCGGAGTATCAGCTCCCGTACGTGCTCGGGTCGGCGCACCAAGGCTGTCTCCCG |
| | CCGTTTCCAGCGGACGTCTTCATGGTCCCTCAGTATGGATACCTCACCCTGAACAACGGAAGTCAAGCGGTGGGACGCTCATCCTTTTACTGCCTGGAGTACTTCCCTTCGCAGATGCTAAGGACTGGAAATAACTTCCAATTCA |
| | GCTATACCTTCGAGGATGTACCTTTTCACAGCAGCTACGCTCACAGCCAGAGTTTGGATCGCTTGATGAATCCTCTTATTGATCAGTATCTGTACTACCTGAACAGAACGCAAGGAACAACCTCTGGAACAACCAACCAATCACG |
| | GCTGCTTTTTAGCCAGGCTGGGCCTCAGTCTATGTCTTTGCAGGCCAGAAATTGGCTACCTGGGCCCTGCTACCGGCAACAGAGACTTTCAAAGACTGCTAACGACAACAACAACAGTAACTTTCCTTGGACAGCGGCCAGCAAA |
| | TATCATCTCAATGGCCGCGACTCGCTGGTGAATCCAGGACCAGCTATGGCCAGTCACAAGGACGATGAAGAAAAATTTTTCCCTATGCACGGCAATCTAATATTTGGCAAAGAAGGGACAACGGCAAGTAACGCAGAATTAGATA |
| | ATGTAATGATTACGGATGAAGAAGAGATTCGTACCACCAATCCTGTGGCAACAGAGCAGTATGGAACTGTGGCAAATAACTTGCAGAGCTCAAATACAGCTCCCACGACTAGAACTGTCAATGATCAGGGGGCCTTACCTGGCAT |
| | GGTGTGGCAAGATCGTGACGTGTACCTTCAAGGACCTATCTGGGCAAAGATTCCTCACACGGATGGACACTTTCATCCTTCTCCTCTGATGGGAGGCTTTGGACTGAAACATCCGCCTCCTCAAATCATGATCAAAAATACTCCG |
| | GTACCGGCAAATCCTCCGACGACTTTCAGCCCGGCCAAGTTTGCTTCATTTATCACTCAGTACTCCACTGGACAGGTCAGCGTGGAAATTGAGTGGGAGCTACAGAAAGAAAACAGCAAACGTTGGAATCCAGAGATTCAGTACA |
| | CTTCCAACTACAACAAGTCTGTTAATGTGGACTTTACTGTAGACACTAATGGTGTTTATAGTGAACCTCGCCCTATTGGAACCCGGTATCTCACACGAAACTTG |
| 458 | >AAV3B265D549A CAPSID AMINO ACID SEQUENCE |
| | MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRP |
| | VDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSDASNDNHYFGYSTPWGYFDENRFHCH |
| | FSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMN |
| | PLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTAASNAELDNVMITDEEEIRTTNPVATEQYGTV |
| | ANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDINGVYSEPR |
| | PIGTRYLTRNL |
| 459 | >AAV3B549A CAPSID NUCLEOTIDE SEQUENCE |
| | ATGGCTGCTGACGGTTATCTTCCAGATTGGCTCGAGGACAACCTTTCTGAAGGCATTCGTGAGTGGTGGGCTCTGAAACCTGGAGTCCCTCAACCCAAAGCGAACCAACAACACCAGGACAACCGTCGGGGTCTTGTGCTTCCGG |
| | GTTACAAATACCTCGGACCCGGTAACGGACTCGACAAAGGAGAGCCGGTCAACGAGGCGGACGCGGCAGCCCTCGAACACGACAAAGCTTACGACCAGCAGCTCAAGGCCGGTGACAACCCGTACCTCAAGTACAACCACGCCGA |
| | CGCCGAGTTTCAGGAGCGTCTTCAAGAAGATACGTCTTTTGGGGGCAACCTTGGCAGAGCAGTCTTCCAGGCCAAAAAGAGGATCCTTGAGCCTCTTGGTCTGGTTGAGGAAGCAGCTAAAACGGCTCCTGGAAAGAAGAGGCCT |
| | GTAGATCAGTCTCCTCAGGAACCGGACTCATCATCTGGTGTTGGCAAATCGGGCAAACAGCCTGCCAGAAAAAGACTAAATTTCGGTCAGACTGGCGACTCAGAGTCAGTCCCAGACCCTCAACCTCTCGGAGAACCACCAGCAG |
| | CCCCCACAAGTTTGGGATCTAATACAATGGCTTCAGGCGGTGGCGCACCAATGGCAGACAATAACGAGGGTGCCGATGGAGTGGGTAATTCCTCAGGAAATTGGCATTGCGATTCCCAATGGCTGGGCGACAGAGTCATCACCAC |
| | CAGCACCAGAACCTGGGCCCTGCCCACTTACAACAACCATCTCTACAAGCAAATCTCCAGCCAATCAGGAGCTTCAAACGACAACCACTACTTTGGCTACAGCACCCCTTGGGGGTATTTTGACTTTAACAGATTCCACTGCCAC |
| | TTCTCACCACGTGACTGGCAGCGACTCATTAACAACAACTGGGGATTCCGGCCCAAGAAACTCAGCTTCAAGCTCTTCAACATCCAAGTTAAAGAGGTCACGCAGAACGATGGCACGACGACTATTGCCAATAACCTTACCAGCA |
| | CGGTTCAAGTGTTTACGGACTCGGAGTATCAGCTCCCGTACGTGCTCGGGTCGGCGCACCAAGGCTGTCTCCCGCCGTTTCCAGCGGACGTCTTCATGGTCCCTCAGTATGGATACCTCACCCTGAACAACGGAAGTCAAGCGGT |
| | GGGACGCTCATCCTTTTACTGCCTGGAGTACTTCCCTTCGCAGATGCTAAGGACTGGAAATAACTTCCAATTCAGCTATACCTTCGAGGATGTACCTTTTCACAGCAGCTACGCTCACAGCCAGAGTTTGGATCGCTTGATGAAT |
| | CCTCTTATTGATCAGTATCTGTACTACCTGAACAGAACGCAAGGAACAACCTCTGGAACAACCAACCAATCACGGCTGCTTTTTAGCCAGGCTGGGCCTCAGTCTATGTCTTTGCAGGCCAGAAATTGGCTACCTGGGCCCTGCT |
| | ACCGGCAACAGAGACTTTCAAAGACTGCTAACGACAACAACAACAGTAACTTTCCTTGGACAGCGGCCAGCAAATATCATCTCAATGGCCGCGACTCGCTGGTGAATCCAGGACCAGCTATGGCCAGTCACAAGGACGATGAAGA |
| | AAAATTTTTCCCTATGCACGGCAATCTAATATTTGGCAAAGAAGGGACAACGGCAAGTAACGCAGAATTAGATAATGTAATGATTACGGATGAAGAAGAGATTCGTACCACCAATCCTGTGGCAACAGAGCAGTATGGAACTGTG |
| | GCAAATAACTTGCAGAGCTCAAATACAGCTCCCACGACTAGAACTGTCAATGATCAGGGGGCCTTACCTGGCATGGTGTGGCAAGATCGTGACGTGTACCTTCAAGGACCTATCTGGGCAAAGATTCCTCACACGGATGGACACT |
| | TTCATCCTTCTCCTCTGATGGGAGGCTTTGGACTGAAACATCCGCCTCCTCAAATCATGATCAAAAATACTCCGGTACCGGCAAATCCTCCGACGACTTTCAGCCCGGCCAAGTTTGCTTCATTTATCACTCAGTACTCCACTGG |
| | ACAGGTCAGCGTGGAAATTGAGTGGGAGCTACAGAAAGAAAACAGCAAACGTTGGAATCCAGAGATTCAGTACACTTCCAACTACAACAAGTCTGTTAATGTGGACTTTACTGTAGACACTAATGGTGTTTATAGTGAACCTCGC |
| | CCTATTGGAACCCGGTATCTCACACGAAACTTG |
| 460 | >AAV3B549A CAPSID AMINO ACID SEQUENCE |
| | MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRP |
| | VDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDENRFHCH |
| | FSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMN |
| | PLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTAASNAELDNVMITDEEEIRTTNPVATEQYGTV |
| | ANNLQSSNTAPTTRTVNDQGALPGMVWQDRDVYLQGPIWAKIPHTDGHFHPSPLMGGFGLKHPPPQIMIKNTPVPANPPTTESPAKFASFITQYSTGQVSVEIEWELQKENSKRWNPEIQYTSNYNKSVNVDETVDINGVYSEPR |
| | PIGTRYLTRNL |
| 461 | >AAV3BQ263Y CAPSID NUCLEOTIDE SEQUENCE |
| | ATGGCTGCTGACGGTTATCTTCCAGATTGGCTCGAGGACAACCTTTCTGAAGGCATTCGTGAGTGGTGGGCTCTGAAACCTGGAGTCCCTCAACCCAAAGCGAACCAACAACACCAGGACAACCGTCGGGGTCTTGTGCTTCCGG |
| | GTTACAAATACCTCGGACCCGGTAACGGACTCGACAAAGGAGAGCCGGTCAACGAGGCGGACGCGGCAGCCCTCGAACACGACAAAGCTTACGACCAGCAGCTCAAGGCCGGTGACAACCCGTACCTCAAGTACAACCACGCCGA |
| | CGCCGAGTTTCAGGAGCGTCTTCAAGAAGATACGTCTTTTGGGGGCAACCTTGGCAGAGCAGTCTTCCAGGCCAAAAAGAGGATCCTTGAGCCTCTTGGTCTGGTTGAGGAAGCAGCTAAAACGGCTCCTGGAAAGAAGAGGCCT |
| | GTAGATCAGTCTCCTCAGGAACCGGACTCATCATCTGGTGTTGGCAAATCGGGCAAACAGCCTGCCAGAAAAAGACTAAATTTCGGTCAGACTGGCGACTCAGAGTCAGTCCCAGACCCTCAACCTCTCGGAGAACCACCAGCAG |
| | CCCCCACAAGTTTGGGATCTAATACAATGGCTTCAGGCGGTGGCGCACCAATGGCAGACAATAACGAGGGTGCCGATGGAGTGGGTAATTCCTCAGGAAATTGGCATTGCGATTCCCAATGGCTGGGCGACAGAGTCATCACCAC |
| | CAGCACCAGAACCTGGGCCCTGCCCACTTACAACAACCATCTCTACAAGCAAATCTCCAGCCAATCAGGAGCTTCAAACGACAACCACTACTTTGGCTACAGCACCCCTTGGGGGTATTTTGACTTTAACAGATTCCACTGCCAC |
| | TTCTCACCACGTGACTGGCAGCGACTCATTAACAACAACTGGGGATTCCGGCCCAAGAAACTCAGCTTCAAGCTCTTCAACATCCAAGTTAAAGAGGTCACGCAGAACGATGGCACGACGACTATTGCCAATAACCTTACCAGCA |
| | CGGTTCAAGTGTTTACGGACTCGGAGTATCAGCTCCCGTACGTGCTCGGGTCGGCGCACCAAGGCTGTCTCCCGCCGTTTCCAGCGGACGTCTTCATGGTCCCTCAGTATGGATACCTCACCCTGAACAACGGAAGTCAAGCGGT |
| | GGGACGCTCATCCTTTTACTGCCTGGAGTACTTCCCTTCGCAGATGCTAAGGACTGGAAATAACTTCCAATTCAGCTATACCTTCGAGGATGTACCTTTTCACAGCAGCTACGCTCACAGCCAGAGTTTGGATCGCTTGATGAAT |
| | CCTCTTATTGATCAGTATCTGTACTACCTGAACAGAACGCAAGGAACAACCTCTGGAACAACCAACCAATCACGGCTGCTTTTTAGCCAGGCTGGGCCTCAGTCTATGTCTTTGCAGGCCAGAAATTGGCTACCTGGGCCCTGCT |
| | ACCGGCAACAGAGACTTTCAAAGACTGCTAACGACAACAACAACAGTAACTTTCCTTGGACAGCGGCCAGCAAATATCATCTCAATGGCCGCGACTCGCTGGTGAATCCAGGACCAGCTATGGCCAGTCACAAGGACGATGAAGA |
| | AAAATTTTTCCCTATGCACGGCAATCTAATATTTGGCAAAGAAGGGACAACGGCAAGTAACGCAGAATTAGATAATGTAATGATTACGGATGAAGAAGAGATTCGTACCACCAATCCTGTGGCAACAGAGCAGTATGGAACTGTG |
| | GCAAATAACTTGCAGAGCTCAAATACAGCTCCCACGACTAGAACTGTCAATGATCAGGGGGCCTTACCTGGCATGGTGTGGCAAGATCGTGACGTGTACCTTCAAGGACCTATCTGGGCAAAGATTCCTCACACGGATGGACACT |
| | TTCATCCTTCTCCTCTGATGGGAGGCTTTGGACTGAAACATCCGCCTCCTCAAATCATGATCAAAAATACTCCGGTACCGGCAAATCCTCCGACGACTTTCAGCCCGGCCAAGTTTGCTTCATTTATCACTCAGTACTCCACTGG |
| | ACAGGTCAGCGTGGAAATTGAGTGGGAGCTACAGAAAGAAAACAGCAAACGTTGGAATCCAGAGATTCAGTACACTTCCAACTACAACAAGTCTGTTAATGTGGACTTTACTGTAGACACTAATGGTGTTTATAGTGAACCTCGC |
| | CCTATTGGAACCCGGTATCTCACACGAAACTTG |
| 462 | >AAV3BQ263Y CAPSID AMINO ACID SEQUENCE |
| | MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRP |
| | VDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSYSGASNDNHYFGYSTPWGYFDENRFHCH |
| | FSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMN |
| | PLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTV |
| | ANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDETVDINGVYSEPR |
| | PIGTRYLTRNL |

### End of Sequences

1. A codon-optimized nucleic acid encoding a human Factor VIII (FVIII) polypeptide, wherein the encoded FVIII polypeptide lacks the B domain, and further comprises an amino acid substitution of Glutamine for Arginine at position 355 (R355Q) and of Glutamine for Arginine at position 581 (R581Q), wherein the nucleic acid comprises the nucleotide sequence set forth in any one of SEQ ID NOs 1, 2, 4, 5, 7-9, 11-15 or 18, or a nucleic acid having at least 90%, 91%, 92%, 93%, 94%, 95%, 96%, 97%, or 98%, or 99% sequence identity thereto.

2. The codon-optimized nucleic acid of claim 1, wherein the nucleic acid comprises the nucleotide sequence set forth in any one of SEQ ID NOs 4, 5, 7, 12-15 or 18, or a nucleic acid having at least 90%, 91%, 92%, 93%, 94%, 95%, 96%, 97%, or 98%, or 99% sequence identity thereto.

3. The codon-optimized nucleic acid of claim 1, wherein the nucleic acid comprises the nucleotide sequence set forth in any one of SEQ ID NOs 4, 5, 13, or 15, or a nucleic acid having at least 90%, 91%, 92%, 93%, 94%, 95%, 96%, 97%, or 98%, or 99% sequence identity thereto.

4. The codon-optimized nucleic acid of claim 1, wherein the nucleic acid comprises the nucleotide sequence set forth in any one of SEQ ID NOs 4 or 5, or a nucleic acid having at least 90%, 91%, 92%, 93%, 94%, 95%, 96%, 97%, or 98%, or 99% sequence identity thereto.

5. The codon-optimized nucleic acid of any one of claims 1-4, wherein the human FVIII polypeptide is a functional variant of the human FVIII polypeptide with the amino acid sequence shown in SEQ ID NO: 19.

6. The codon-optimized nucleic acid of claim 5, wherein the functional variant has at least 60%, or 70%, or 80%, 85% or 90% or 95%, or 98%, or 99% sequence identity to the amino acid sequence shown in SEQ ID NO: 19.

7. The codon-optimized nucleic acid of any one of claims 1-6, wherein the B domain of the encoded FVIII polypeptide has been replaced by a peptide linker.

8. The codon-optimized nucleic acid of any one of claims 1-7, wherein the encoded FVIII polypeptide lacks both the amino acid substitution of Glutamine for Arginine at position 355 (R355Q) and of Glutamine for Arginine at position 581 (R581Q).

9. The codon-optimized nucleic acid of any one of claims 1-7, wherein the encoded FVIII polypeptide lacks the amino acid substitution of Glutamine for Arginine at position 355 (R355Q).

10. The codon-optimized nucleic acid of any one of claims 1-7, wherein the encoded FVIII polypeptide lacks the amino acid substitution of Glutamine for Arginine at position 581 (R581Q).

11. The codon-optimized nucleic acid of any one of claims 1-10, that is comprised within a nucleic acid construct that further comprises viral sequence elements that facilitate integration and expression.

12. An expression cassette containing the codon-optimized nucleic acid of any one of claims 1-11, operably linked to a constitutive promoter.

13. The expression cassette of claim 11, wherein the constitutive promoter is a TTR promoter.

14. The expression cassette of claim 12, wherein the TTR promoter comprises a nucleic acid sequence of SEQ ID NO: 431.

15. The expression cassette of any one of claims 10-13, wherein the promoter is a liver-specific promoter.

16. The expression cassette of claim 15, wherein the liver specific promoter is selected from any of: SEQ ID NOS: 86, 88, 91-96, 146-150, 439-441, or 481-500, or a liver specific promoter having at least 80% sequence identity to SEQ ID NOs: 86, 88, 91-96, 146-150, 439-441, or 481-500.

17. The expression cassette of claim 15, wherein the liver specific promoter is selected from any of: SEQ ID NOS: 98 or 99, or a liver specific promoter having at least 80% sequence identity to SEQ ID NOs: 98 or 99.

18. The expression cassette of claim 15, wherein the liver specific promoter is SEQ ID NOS: 97, or a liver specific promoter having at least 80% sequence identity to SEQ ID NO: 97.

19. The expression cassette of any one of claims 12-18, further comprising one or more additional regulatory elements and/or a poly A sequence.

20. The expression cassette of claim 19, wherein the one or more additional regulatory elements is selected from the group consisting of an enhancer, a 5′ untranslated region (5′UTR), an intron, a reverse RNA pol II terminator sequence, and combinations thereof.

21. A recombinant adeno-associated virus (rAAV) vector comprising in its genome the expression cassette of any one of claims 12-20.

22. A recombinant adeno-associated virus (rAAV) vector comprising in its genome:
c) 5′ and 3′ AAV inverted terminal repeats (ITR) sequences; and
d) located between the 5′ and 3′ ITRs, the expression cassette specified in any one of claims 12-20.

23. The rAAV vector of claims 21-22, wherein the AAV genome further comprises at least one of:
g) a 5′ ITR;
h) an 5′ UTR sequence;
i) an intron;
j) a poly A sequence;
k) a reverse RNA pol II terminator sequence; and
l) a 3′ ITR.

24. The rAAV vector of any one of claims 21-23, wherein the AAV genome comprises, in the 5′ to 3′ direction:
a) a 5′ ITR;
b) liver specific promoter
b) a 5′ UTR sequence;
c) an intron;
d) a codon-optimized nucleic acid specified in any one of claims 1-10;
d) a poly A sequence;
e) a reverse RNA pol II terminator sequence; and
f) a 3′ ITR.

25. The rAAV vector of any one of claims 23-24, wherein the 5′ UTR sequence comprises SEQ ID NO: 41, or a nucleic acid having at least 90% sequence identity to SEQ ID NO: 41.

26. The rAAV vector of any one of claims 23-24, wherein the 5′ UTR sequence comprises SEQ ID NO: 40, or a nucleic acid having at least 90% sequence identity to SEQ ID NO: 40.

27. The rAAV vector of any one of claims 23-24, wherein the intron is selected from the group consisting of a MVM sequence, a HBB2 sequence, an CMVIE intron sequence, a UBC intron sequence, and a SV40 sequence.

28. The rAAV vector of any one of claims 23-24, wherein the 3′ UTR sequence is located 3′ of the codon-optimized nucleic acid and 5′ of the 3′ ITR sequence, or is located between the codon-optimized nucleic acid and the poly A sequence.

29. The rAAV vector of any one of claims 23-16, wherein the heterologous, codon-optimized nucleic acid sequence further comprises a 3′ intron sequence, wherein the 3′ intron sequence is located 3′ of the nucleic acid encoding the FVIII polypeptide and 5′ of the 3′ ITR sequence, or is located between the nucleic acid encoding the FVIII polypeptide and the poly A sequence.

30. The rAAV vector of any one of claims 22-29, wherein at least one of the 5′ ITR or 3′ITR comprises an insertion, deletion or substitution.

31. The rAAV vector of claim 22-30, wherein one or more CpG islands in the ITR are removed.

32. The rAAV vector of any one of claims 23-31, wherein the poly A sequence is a full length HGF poly A sequence.

33. The rAAV vector of any one of claims 23-31, wherein poly A sequence is selected from SEQ ID NO: 42-44 or 514, or a nucleic acid sequence at least 80% sequence identity to SEQ ID NOS: 42-44 or 514.

34. The rAAV vector of any one of claims 23 to 33, wherein the reverse RNA pol II terminator sequence is SEQ ID NO: 45, or a nucleic acid sequence at least 80% sequence identity to SEQ ID NOS: 45.

35. The rAAV vector of any one of claims 21-34, wherein the rAAV vector is a chimeric AAV vector, haploid AAV vector, a hybrid AAV vector or polyploid AAV vector.

36. The rAAV vector of any one of claims 22-34, wherein the rAAV vector is a rational haploid vector, a mosaic AAV vector, a chemically modified AAV vector, or a AAV vector from any AAV serotypes.

37. The rAAV vector of any one of claims 22-36, wherein the rAAV vector is selected from the group consisting of: a AAVXL32 vector, a AAVXL32.1 vector, a AAV8 vector, or a haploid AAV8 vector comprising at least one AAV8 capsid protein.

38. The rAAV vector of any one of claims 22-36, that has a capsid comprising capsid proteins from a serotype shown in Table 3 or a chimera thereof.

39. The rAAV vector of claim 38, wherein the capsid proteins are serotype AAV3b.

40. The rAAV vector of claim 39, wherein the AAV3b serotype capsid protein comprises one or more mutations selected from any of: 265D, 549A, Q263Y

41. The rAAV vector of claim 39, wherein the AAV3b serotype is selected from any of: AAV3b265D, AAV3b265D549A, AAV3b549A or AAV3bQ263Y, or AAV3bSASTG.

42. A pharmaceutical composition comprising the rAAV vector of any one of claims 21-41 in a pharmaceutically acceptable carrier.

43. A method for treating a subject in need of FVIII, the method comprising administering the rAAV vectors of any one of claims 21-41 or the pharmaceutical composition of claim 42, or the expression cassette of any one of claims 12-20 or the codon-optimized nucleic acid of any one of claims 1-11, to the subject.

44. A method for treating hemophilia A, the method comprising administering the rAAV vectors of any one of claims 21-41 or the pharmaceutical composition of claim 42, or the expression cassette of any one of claims 12-20 or the codon-optimized nucleic acid of any one of claims 1-11, to the subject.

45. The method of any one of claim 43 or 44, wherein the AAV vector is manufactured from the plasmid of SEQ ID NO: 27.

46. The method of any one of claims 43-45, wherein the encoded FVIII polypeptide is secreted from the subject's liver.

47. The method of any one of claims 43-46, wherein administering to the subject is by systemic administration.

48. The method of claim 47, wherein the systemic administration is by intravenous administration.

49. The method of any one of claims 43-46, wherein administering to the subject is by local administration.

50. The method of claim 49, wherein the local administration is by injection to the liver.

51. The method of any one of claims 43-50, where the rAAV vector is administered at a dosage range of between 1.0E9 vg/kg to 5.0E12 vg/kg.

52. Use of a rAAV vector in the preparation of a medicament for treating subject in need of FVIII, the medicament comprising the rAAV vector specified in of any one of claims 21-41.

53. Use of a rAAV vector in the preparation of a medicament for treating hemophilia A, the medicament comprising the rAAV vector specified in of any one of claims 21-41.

54. An expression cassette containing the codon-optimized nucleic acid of any one of claims 1-11, operably linked to a liver-specific promoter, wherein the liver specific promoter is selected from any of: SEQ ID NOS: 481-500, or a liver specific promoter having at least 80% sequence identity to SEQ ID NOs: 481-500.

55. An expression cassette containing the codon-optimized nucleic acid of any one of claims 1-11, operably linked to a liver-specific promoter, wherein the liver specific promoter is selected from any of: SEQ ID NOS: 481-483, or a liver specific promoter having at least 80% sequence identity to SEQ ID NOs: 481-483.

56. A recombinant adeno-associated virus (rAAV) vector comprising in its genome the expression cassette of claim 54 or 55.


SEQUENCE LISTING (521 sequences)

SEQ ID NO: 1 - DNA, 4376 bp, synthetic construct
  source 1..4376: mol_type = other DNA
atgcagattgagctgagcacctgcttcttcctgtgcctgctgagattctgcttctctgccaccagaagatactacctgggtgcagtggagctgagctgggactacatgcagagtgacctgggtgagctgcctgttgatgccagattcccccccagagtgcccaagagcttccccttcaacacctctgttgtgtacaagaagaccctgtttgttgagaattcactgaccacctgttcaacattgccaagcccagacccccctggatgggcctgctgggccccaccatccaggcagaggtgtatgatactgtggtgatcaccctgaagaacatggccagccacccagttagcctgcatgcagtgggggtcagctactggaaggcctcagaaggggctgagtatgatgatcagaccagccagagagagaaggaggatgacaaggtgttcccagggggtagccacacctatgtatggcaggtgctgaaggagaatggccccatggcctctgaccccctgtgcctgacctacagctacctgagccatgtggacctggtgaaggacctgaacagtggactgattggtgccctgctggtgtgcagagagggcagcctggccaaggagaagacccagaccctgcacaagttcatcctgctgtttgctgtgtttgatgaaggcaagagctggcacagtgagaccaagaacagcctgatgcaggacagagatgctgcatcagctagagcctggcccaagatgcacactgtcaatggttatgtaaacagaagcctgcctggcctgattggctgccacagaaagtcagtatactggcatgttataggcatgggcaccaccccagaggtgcacagcatcttcctggagggccacaccttcctggtgagaaaccacagacaggccagcctggagatcagccccatcaccttcctgacagcccagaccctgctgatggacctgggccagttcctgctgttctgccacatcagcagccaccagcatgatgggatggaggcctatgtaaaggtggacagctgcccagaggagccccagctgcagatgaagaacaatgaagaggctgaagactatgatgatgatctgactgattcagagatggatgttgtgagatttgatgatgacaacagccccagcttcatccagatcagatcagtggccaagaagcaccccaagacctgggtgcactacatagctgcagaggaggaggactgggactatgcccccctggtgctggccccagatgatagaagctacaagagccagtacctgaacaatggcccccagagaatagggagaaagtacaagaaggtgagattcatggcctacacagatgagaccttcaagaccagagaggccatccagcatgagtcaggtatcctgggccccctgctgtatggggaggtgggtgacaccctgctgatcatcttcaagaaccaggccagcagaccctacaacatctacccccatgggatcacagatgtcagacccctgtacagcagaagactgcccaagggagtcaagcacctgaaggacttccccatcctgcctggagaaatcttcaagtacaagtggacagtgactgtggaggatggtcccaccaagagtgaccccagatgcctgaccagatactacagcagctttgtcaacatggagagagacctggccagtggcctgataggccccctgctgatctgctacaaggagtctgtggaccagcagggcaaccagatcatgtctgacaagagaaatgttatcctgttcagtgtatttgatgagaacagaagctggtacctgactgagaacatccagagattcctgcccaacccagctggggtccagctggaggaccctgagttccaggccagcaacatcatgcacagcatcaatggctatgtgtttgacagcctgcagctgtctgtctgcctgcatgaagtggcctactggtacatcctgagcataggagcccagacagacttcctgagtgtattcttctctggctacaccttcaagcacaagatggtgtatgaggacaccctgaccctgttccccttctctggagagactgtattcatgagcatggagaaccctggcctgtggatcctgggctgccacaactctgacttcagaaacagaggcatgactgccctgctgaaggtgagcagctgtgataagaacacaggggactactatgaggacagctatgaggacatctcagcctacctgctgagcaagaacaatgccattgagcccagaagcttcagccagaacccccctgtgctgaagagacaccagagagagatcaccagaaccaccctgcagagtgaccaggaggagattgactatgatgacaccatctctgttgagatgaagaaggaggactttgacatctatgatgaggatgagaaccagagccccagaagcttccagaagaagaccagacactacttcattgcagctgtggagagactgtgggactatggcatgagcagcagcccccatgtgctgagaaacagagcccagtcaggctctgtgccccagttcaagaaggtggtgttccaggagttcacagatgggagcttcacccagcccctgtacagaggtgaactgaatgagcacctgggcctgctgggcccctacatcagagctgaagtggaggacaacatcatggtgaccttcagaaaccaggccagcagaccctacagcttctacagcagcctgatcagctatgaagaggaccagagacagggtgctgagcccagaaagaactttgtgaagcccaatgaaaccaagacctacttctggaaggtgcagcaccacatggcccccaccaaggatgaatttgactgcaaggcctgggcctacttctcagatgtagacctggagaaggatgttcactcaggcctgattgggcccctgctggtgtgccacaccaacaccctgaacccagcccatggtagacaggtgacagtgcaggagtttgccctgttcttcaccatctttgatgagaccaagagctggtacttcactgagaacatggagagaaactgcagagccccctgcaacatccagatggaggaccccaccttcaaggagaactacagattccatgccatcaatgggtacatcatggacaccctgccaggcctggtgatggcccaggaccagagaatcagatggtacctgctgagcatgggcagcaatgagaacatccacagcatccacttcagtggtcatgtcttcacagtgagaaagaaggaggagtacaagatggccctgtacaacctgtacccaggggtgtttgaaactgtagagatgctgcccagcaaggcaggcatctggagagtggagtgcctgataggagaacacctgcatgcaggaatgagcaccctgttcctggtgtacagcaacaagtgccagacccccctgggcatggcctctgggcacatcagagacttccagatcactgcctctggccagtatgggcagtgggcccccaagctggccagactgcactactcaggtagcatcaatgcctggagcaccaaggagcccttcagctggatcaaggtggacctgctggcccccatgatcatccatggtatcaagacccagggtgccagacagaagttcagcagcctgtacatcagccagttcatcatcatgtacagcctggatggcaagaagtggcagacctacagaggcaacagcactggcaccctgatggtgttctttggcaatgtggacagctctggcatcaagcacaacatcttcaacccccccatcatagccagatacatcagactgcaccccacccactacagcatcagaagcaccctgagaatggagctgatgggctgtgatctgaacagctgcagcatgcccctgggcatggagagcaaggccatctctgatgctcagatcactgcaagcagctacttcaccaacatgtttgccacctggagccccagcaaggccagactgcacctgcagggcagaagcaatgcctggagaccccaggtgaacaaccccaaggagtggctgcaggtggacttccagaagaccatgaaggtgacaggtgtcaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgagaatccacccccagagctgggtgcaccagattgccctgagaatggaggtgctgggctgtgaggcccaggacctgtactga

SEQ ID NO: 2 - DNA, 4374 bp, synthetic construct
  source 1..4374: mol_type = other DNA
atgcagattgagctgagcacctgcttcttcctgtgcctgctgagattctgcttcagtgccaccagaagatactacctgggagcagtggagctgagctgggactacatgcagagtgatctgggagagctgccagtagatgccagattcccccccagagtgcccaagagcttccccttcaacacctcagtggtgtacaagaagaccctgtttgttgagttcactgaccacctgttcaacattgcaaagcccagacccccctggatgggcctgctgggccccaccatccaggcagaagtgtatgacacagtagtgatcaccctgaagaacatggccagccaccctgtcagcctgcatgcagttggtgtaagctactggaaggcctctgagggtgctgaatatgatgaccagaccagccagagagagaaggaggatgataaggtgttcccagggggcagccacacctatgtttggcaggtgctgaaggagaatgggcccatggcctctgatcccctgtgcctgacctacagctacctgagccatgtggacctggtgaaggacctgaactctgggctgataggagccctgctggtgtgcagagagggcagcctggccaaggagaagacccagaccctgcacaagttcatcctgctgtttgctgtgtttgatgaaggcaagagctggcacagtgaaaccaagaacagcctgatgcaggacagagatgcagctagtgccagagcctggcccaagatgcacacagtaaatggttatgtgaacagaagcctgcctggcctgataggctgccacagaaagtctgtgtactggcatgttataggtatgggcaccacccctgaggtgcacagcatcttcctggagggccacaccttcctggtgagaaaccacagacaggccagcctggagatcagccccatcaccttcctgactgcccagaccctgctgatggacctgggccagttcctgctgttctgccacatcagcagccaccagcatgatggaatggaggcctatgtcaaggtggacagctgcccagaggagccccagctgcagatgaagaacaatgaggaggcagaggactatgatgatgacctgactgacagtgaaatggatgtggtcagatttgatgatgataacagccccagcttcatccagatcagatcagtggccaagaagcaccccaagacctgggtgcactacattgcagctgaggaggaggactgggactatgcacccctggtgctggccccagatgatagaagctacaagagccagtacctgaacaatggaccccagagaattggcagaaagtacaagaaggtgagattcatggcctacactgatgagaccttcaagaccagagaggccatccagcatgaatcaggcatcctgggccccctgctgtatggggaggtgggagacaccctgctgatcatcttcaagaaccaggccagcagaccctacaacatctacccccatggaatcacagatgttagacccctgtacagcagaagactgcccaagggggtgaagcacctgaaggacttccccatcctgcctggggagatcttcaagtacaagtggacagttacagtagaggatggccccaccaagagtgaccccagatgcctgaccagatactacagcagctttgtaaacatggagagagacctggcctctggcctgataggccccctgctgatctgctacaaggagtcagttgaccagcagggcaaccagatcatgagtgataagagaaatgtcatcctgttcagtgtgtttgatgagaacagaagctggtacctgacagagaacatccagagattcctgcccaaccctgctggagtgcagctggaggacccagaattccaggccagcaacatcatgcacagcatcaatggttatgtctttgatagcctgcagctgtcagtttgcctgcatgaggtggcctactggtacatcctgagcattggggcccagactgacttcctgagtgtattcttcagtggctacaccttcaagcacaagatggtgtatgaggacaccctgaccctgttccccttctctggagagactgtgttcatgagcatggagaaccctgggctgtggatcctgggctgccacaactcagacttcagaaacagaggcatgacagcactgctgaaggtgagcagctgtgataagaacactggtgactactatgaggacagctatgaagacatctcagcctacctgctgagcaagaacaatgccatagagcccagaagcttcagccagaaccccccagttctgaagagacaccagagagagatcaccagaaccaccctgcagagtgaccaggaggagatagattatgatgataccatctcagttgagatgaagaaggaggactttgatatctatgatgaggatgagaaccagagccccagaagcttccagaagaagaccagacactacttcatagcagctgtggagagactgtgggactatggcatgagcagcagcccccatgttctgagaaacagagcccagagtggatcagtaccccagttcaagaaggtggtgttccaggagttcactgatgggagcttcacccagcccctgtacagaggtgaactgaatgagcacctgggcctgctgggcccctacatcagagctgaagtggaggacaacatcatggtgaccttcagaaaccaggccagcagaccctacagcttctacagcagcctgatcagctatgaggaggaccagagacagggagctgagcccagaaagaactttgtcaagcccaatgaaaccaagacctacttctggaaggtgcagcaccacatggcccccaccaaggatgagtttgactgcaaggcctgggcctacttcagtgatgtagacctggagaaggatgtccactcaggcctgattggccccctgctggtgtgccacaccaacaccctgaacccagcacatgggagacaggtgactgtacaggagtttgctctgttcttcaccatctttgatgagaccaagagctggtacttcacagaaaacatggagagaaactgcagagccccctgcaacatccagatggaggaccccaccttcaaggagaactacagattccatgccatcaatggttacatcatggacaccctgcctggcctggtgatggcccaggaccagagaatcagatggtacctgctgagcatgggcagcaatgaaaacatccacagcatccacttctcagggcatgtgttcactgtgagaaagaaggaggagtacaagatggccctgtacaacctgtacccaggtgtgtttgaaactgtggagatgctgcccagcaaggcaggcatctggagagtggagtgcctgataggggagcacctgcatgcagggatgagcaccctgttcctggtgtacagcaacaagtgccagacccccctgggcatggcctcaggccacatcagagacttccagatcacagcttctggacagtatggccagtgggcccccaagctggccagactgcactacagtggcagcatcaatgcctggagcaccaaggagcccttcagctggatcaaggtggacctgctggcccccatgatcatccatggcatcaagacccagggagccagacagaagttcagcagcctgtacatcagccagttcatcatcatgtacagcctggatggcaagaagtggcagacctacagaggcaacagcacaggcaccctgatggtgttctttgggaatgtggacagcagtggaatcaagcacaacatcttcaacccccccatcattgcaagatacatcagactgcaccccacccactacagcatcagaagcaccctgagaatggagctgatgggctgtgacctgaacagctgcagcatgcccctgggcatggagagcaaggccatctctgatgcccagatcacagctagcagctacttcaccaacatgtttgccacctggagccccagcaaggccagactgcacctgcagggcagaagcaatgcctggagaccccaggtgaacaaccccaaggagtggctgcaggtggacttccagaagaccatgaaggtgactggtgttaccacccagggggtcaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgagaatccacccccagagctgggtgcaccagatagcactgagaatggaggtgctgggctgtgaggcccaggacctgtactga

SEQ ID NO: 3 - DNA, 4374 bp, synthetic construct
  source 1..4374: mol_type = other DNA
atgcaaattgagttgtccacctgcttcttcttgtgcttgttgaggttctgcttcagtgccaccaggaggtactacttgggggctgtggagttgtcctgggactacatgcagtcagacttgggggagttgcctgtggatgccaggttcccccccagggtccccaagtccttccccttcaacaccagtgtggtctacaagaagaccttgtttgtggagttcactgaccacttgttcaacattgccaagcccaggcccccctggatgggcttgttgggccccaccatccaggctgaggtctatgacacagtggtcatcaccttgaagaacatggcctcccaccctgtctccttgcatgcagtgggggtctcctactggaaggccagtgagggggctgagtatgatgaccagacctcccagagggagaaggaggatgacaaggtcttccctgggggctcccacacctatgtctggcaggtcttgaaggagaatggccccatggcctctgaccccttgtgcttgacctactcctacttgtcccatgtggacttggtcaaggacttgaactcaggcttgattggggccttgttggtctgcagggagggctccttggccaaggagaagacccagaccttgcacaagttcatcttgttgtttgcagtctttgatgagggcaagtcctggcacagtgagaccaagaactccttgatgcaggacagggatgctgccagtgccagggcctggcccaagatgcacactgtcaatggctatgtcaacaggtccttgcctggcttgattggctgccacaggaagtcagtctactggcatgtcattggcatgggcaccacccctgaggtccactccatcttcttggagggccacaccttcttggtcaggaaccacaggcaggcctccttggagatctcccccatcaccttcttgacagcccagaccttgttgatggacttgggccagttcttgttgttctgccacatctcctcccaccagcatgatggcatggaggcctatgtcaaggtggactcctgccctgaggagccccagttgcagatgaagaacaatgaggaggcagaggactatgatgatgacttgactgactctgagatggatgtggtcaggtttgatgatgacaactccccctccttcatccagatcaggagtgtggccaagaagcaccccaagacctgggtccactacatagcagcagaggaggaggactgggactatgcccccttggtcttggcccctgatgacaggtcctacaagtcccagtacttgaacaatggcccccagaggattggcaggaagtacaagaaggtcaggttcatggcctacacagatgagaccttcaagaccagggaggccatccagcatgagtcaggcatcttgggccccttgttgtatggggaggtgggggacaccttgttgatcatcttcaagaaccaggcctccaggccctacaacatctacccccatggcatcactgatgtcaggcccttgtactccaggaggttgcccaagggggtcaagcacttgaaggacttccccatcttgccaggggagatcttcaagtacaagtggacagtcactgtggaggatggccccaccaagtcagaccccaggtgcttgaccaggtactactcctcctttgtcaacatggagagggacttggccagtggcttgattggccccttgttgatctgctacaaggagtctgtggaccagcagggcaaccagatcatgtcagacaagaggaatgtcatcttgttcagtgtctttgatgagaacaggtcctggtacttgacagagaacatccagaggttcttgcccaacccagctggggtccagttggaggaccctgagttccaggcctccaacatcatgcactccatcaatggctatgtctttgactccttgcagttgtcagtctgcttgcatgaggtggcctactggtacatcttgtccattggggcccagactgacttcttgtctgtcttcttcagtggctacaccttcaagcacaagatggtctatgaggacaccttgaccttgttccccttcagtggggagactgtcttcatgtccatggagaacccaggcttgtggatcttgggctgccacaactcagacttcaggaacaggggcatgacagccttgttgaaggtctcctcctgtgacaagaacacaggggactactatgaggactcctatgaggacatctcagcctacttgttgtccaagaacaatgccatagagcccaggtccttctcccagaacccccctgtcttgaagaggcaccagagggagatcaccaggaccaccttgcagtcagaccaggaggagatagactatgatgacaccatctcagtggagatgaagaaggaggactttgacatctatgatgaggatgagaaccagtcccccaggtccttccagaagaagaccaggcactacttcattgcagcagtggagaggttgtgggactatggcatgtcctcctccccccatgtcttgaggaacagggcccagtcaggcagtgtcccccagttcaagaaggtggtcttccaggagttcactgatggctccttcacccagcccttgtacaggggggagttgaatgagcacttgggcttgttgggcccctacatcagggctgaggtggaggacaacatcatggtcaccttcaggaaccaggcctccaggccctactccttctactcctccttgatctcctatgaggaggaccagaggcagggggcagagcccaggaagaactttgtcaagcccaatgagaccaagacctacttctggaaggtccagcaccacatggcccccaccaaggatgagtttgactgcaaggcctgggcctacttctctgatgtggacttggagaaggatgtccactctggcttgattggccccttgttggtctgccacaccaacaccttgaaccctgcccatggcaggcaggtcactgtccaggagtttgccttgttcttcaccatctttgatgagaccaagtcctggtacttcacagagaacatggagaggaactgcagggccccctgcaacatccagatggaggaccccaccttcaaggagaactacaggttccatgccatcaatggctacatcatggacaccttgccaggcttggtcatggcccaggaccagaggatcaggtggtacttgttgtccatgggctccaatgagaacatccactccatccacttcagtggccatgtcttcactgtcaggaagaaggaggagtacaagatggccttgtacaacttgtacccaggggtctttgagacagtggagatgttgccctccaaggcaggcatctggagggtggagtgcttgattggggagcacttgcatgctggcatgtccaccttgttcttggtctactccaacaagtgccagacccccttgggcatggcctctggccacatcagggacttccagatcactgcctcaggccagtatggccagtgggcccccaagttggccaggttgcactacagtggctccatcaatgcctggtccaccaaggagcccttctcctggatcaaggtggacttgttggcccccatgatcatccatggcatcaagacccagggggccaggcagaagttctcctccttgtacatctcccagttcatcatcatgtactccttggatggcaagaagtggcagacctacaggggcaactccacaggcaccttgatggtcttctttggcaatgtggactcctctggcatcaagcacaacatcttcaacccccccatcattgccaggtacatcaggttgcaccccacccactactccatcaggtccaccttgaggatggagttgatgggctgtgacttgaactcctgctccatgcccttgggcatggagtccaaggccatctcagatgcccagatcactgcctcctcctacttcaccaacatgtttgccacctggtccccctccaaggccaggttgcacttgcagggcaggtccaatgcctggaggccccaggtcaacaaccccaaggagtggttgcaggtggacttccagaagaccatgaaggtcacaggggtcaccacccagggggtcaagtccttgttgacctccatgtatgtcaaggagttcttgatctcctcctcccaggatggccaccagtggaccttgttcttccagaatggcaaggtcaaggtcttccagggcaaccaggactccttcacccctgtggtcaactccttggacccccccttgttgaccaggtacttgaggatccacccccagtcctgggtccaccagattgccttgaggatggaggtcttgggctgtgaggcccaggacttgtactga

SEQ ID NO: 4 - DNA, 4374 bp, synthetic construct
  source 1..4374: mol_type = other DNA
atgcagatagagctgagcacctgcttcttcctgtgcctgctgagattctgcttctcagccaccagaagatactacctgggggctgtagagctgagctgggactacatgcagtcagacctgggtgagctgccagtggatgccagattcccccccagagtgcccaagagcttccccttcaacacctctgtggtgtacaagaagaccctgtttgtggagttcactgatcacctgttcaacatagctaagcccagacccccctggatgggcctgctgggccccaccatccaggctgaggtgtatgatacagtggtgatcaccctgaagaacatggccagccaccctgtgagcctgcatgcagtgggtgtgagctactggaaggcctctgagggtgctgaatatgatgaccagaccagccagagagagaaggaggatgataaggtgttccctgggggcagccacacctatgtgtggcaggtgctgaaggagaatggacccatggccagtgaccccctgtgcctgacctacagctacctgagccatgtagacctggtgaaggacctgaactctggactgattggggcactgctggtgtgcagagagggcagcctggccaaggagaagacccagaccctgcacaagttcatcctgctgtttgctgtgtttgatgagggcaagagctggcactctgaaaccaagaacagcctgatgcaggacagagatgctgcctcagcaagagcctggcccaagatgcacactgtgaatggctatgttaacagaagcctgcctgggctgattgggtgccacagaaagagtgtgtactggcatgtaataggaatgggcaccaccccagaggtgcacagcatcttcctggagggccacaccttcctggtgagaaaccacagacaggccagcctggagatcagccccatcaccttcctgactgctcagaccctgctgatggacctgggccagttcctgctgttctgccacatcagcagccaccagcatgatggaatggaggcctatgtaaaggtggacagctgccctgaggagccccagctgcagatgaagaacaatgaggaggcagaggactatgatgatgacctgactgacagtgagatggatgtggtgagatttgatgatgataacagccccagcttcatccagatcagatctgtagccaagaagcaccccaagacctgggtgcactacatagctgcagaagaggaggactgggactatgcccccctggtgctggcccctgatgatagaagctacaagagccagtacctgaacaatggtccccagagaatagggagaaagtacaagaaggtgagattcatggcctacactgatgagaccttcaagaccagagaggccatccagcatgagtcaggcatcctgggccccctgctgtatggagaagtgggtgacaccctgctgatcatcttcaagaaccaggccagcagaccctacaacatctacccccatggcatcactgatgtcagacccctgtacagcagaagactgcccaagggtgtgaagcacctgaaggacttccccatcctgccaggtgagatcttcaagtacaagtggacagtgactgtagaggatgggcccaccaagtcagaccccagatgcctgaccagatactacagcagctttgtgaacatggagagagacctggccagtggcctgattggccccctgctgatctgctacaaggagtcagttgaccagcagggcaaccagatcatgtctgacaagagaaatgtcatcctgttcagtgtgtttgatgagaacagaagctggtacctgactgagaacatccagagattcctgcccaacccagcaggagttcagctggaggaccctgagttccaggccagcaacatcatgcacagcatcaatggctatgtttttgacagcctgcagctgtcagtgtgcctgcatgaagtggcctactggtacatcctgagcattggtgcccagacagatttcctgagtgtcttcttctcaggctacaccttcaagcacaagatggtgtatgaggacaccctgaccctgttccccttctctggggaaacagtgttcatgagcatggagaaccctggtctgtggatcctgggctgccacaactcagacttcagaaacagaggcatgacagcactgctgaaggtgagcagctgtgacaagaacacaggtgactactatgaagacagctatgaggacatctcagcctacctgctgagcaagaacaatgccattgagcccagaagcttcagccagaacccccctgtgctgaagagacaccagagagagatcaccagaaccaccctgcagtcagaccaggaggagattgactatgatgacaccatcagtgtagaaatgaagaaggaggactttgacatctatgatgaggatgaaaaccagagccccagaagcttccagaagaagaccagacactacttcatagctgcagttgagagactgtgggactatggaatgagcagcagcccccatgtgctgagaaacagagcccagtctggctcagttccccagttcaagaaggtggtgttccaggagttcacagatgggagcttcacccagcccctgtacagaggggagctgaatgagcacctgggcctgctgggcccctacatcagagcagaggtggaggacaacatcatggtgaccttcagaaaccaggccagcagaccctacagcttctacagcagcctgatcagctatgaagaggaccagagacagggagcagaacccagaaagaactttgttaagcccaatgaaaccaagacctacttctggaaggtgcagcaccacatggcccccaccaaggatgaatttgactgcaaggcctgggcctacttctctgatgttgacctggagaaggatgtgcactcaggcctgataggccccctgctggtgtgccacaccaacaccctgaacccagctcatgggagacaggtgactgttcaggagtttgccctgttcttcaccatctttgatgagaccaagagctggtacttcacagagaacatggagagaaactgcagagccccctgcaacatccagatggaggaccccaccttcaaggagaactacagattccatgccatcaatggttacatcatggacaccctgcctgggctggtgatggcccaggaccagagaatcagatggtacctgctgagcatgggcagcaatgaaaacatccacagcatccacttctctggccatgtgttcactgtcagaaagaaggaggagtacaagatggccctgtacaacctgtaccctggagtatttgagactgtggagatgctgcccagcaaggcaggtatctggagagtggagtgcctgattggggagcacctgcatgcaggtatgagcaccctgttcctggtgtacagcaacaagtgccagacccccctgggcatggcctctggacacatcagagacttccagatcactgccagtggccagtatggccagtgggcccccaagctggccagactgcactactcagggagcatcaatgcctggagcaccaaggagcccttcagctggatcaaggtggacctgctggcccccatgatcatccatggtatcaagacccagggtgccagacagaagttcagcagcctgtacatcagccagttcatcatcatgtacagcctggatggcaagaagtggcagacctacagaggcaacagcacaggcaccctgatggtgttctttggcaatgtagacagctctggcatcaagcacaacatcttcaacccccccatcatagctagatacatcagactgcaccccacccactacagcatcagaagcaccctgagaatggagctgatgggctgtgacctgaacagctgcagcatgcccctgggcatggagagcaaggccatctcagatgctcagatcactgccagcagctacttcaccaacatgtttgccacctggagccccagcaaggccagactgcacctgcagggcagaagcaatgcctggagaccccaggtgaacaaccccaaggagtggctgcaggtggacttccagaagaccatgaaggtgacaggagtgaccacccagggggtgaagagcctgctgaccagcatgtatgtgaaggagttcctgatcagcagcagccaggatggacaccagtggaccctgttcttccagaatggaaaggtgaaggtgttccagggcaaccaggacagcttcacccctgtggtgaacagcctggacccccccctgctgaccagatacctgagaatccacccccagagctgggtgcaccagattgccctgagaatggaggtgctgggctgtgaggcccaggacctgtactga

SEQ ID NO: 5 - DNA, 4374 bp, synthetic construct
  source 1..4374: mol_type = other DNA
atgcagattgagctgagcacctgcttcttcctgtgcctgctgagattctgcttctctgctaccagaagatactacctgggagctgtggagctgagctgggactacatgcagtcagacctgggtgaactgcctgtggatgccagattcccccccagagtgcccaagagcttccccttcaacaccagtgttgtctacaagaagaccctgtttgtggagttcacagaccacctgttcaacattgccaagcccagacccccctggatgggcctgctgggccccaccatccaggctgaagtgtatgatacagtggtgatcaccctgaagaacatggccagccacccagtgagcctgcatgcagtgggtgtgagctactggaaggcctctgaaggtgcagagtatgatgaccagaccagccagagagagaaggaggatgacaaggtgttccctgggggcagccacacctatgtgtggcaggtgctgaaggagaatggccccatggccagtgatcccctgtgcctgacctacagctacctgagccatgttgacctggtgaaggacctgaactcaggtctgattggagccctgctggtgtgcagagagggcagcctggccaaggagaagacccagaccctgcacaagttcatcctgctgtttgctgtctttgatgaaggcaagagctggcacagtgagaccaagaacagcctgatgcaggacagagatgcagcctctgccagagcctggcccaagatgcacacagtaaatggctatgtgaacagaagcctgccagggctgattggctgccacagaaagtctgtttactggcatgtgattgggatgggcaccacccctgaagtgcacagcatcttcctggagggccacaccttcctggtgagaaaccacagacaggccagcctggagatcagccccatcaccttcctgacagcacagaccctgctgatggacctgggccagttcctgctgttctgccacatcagcagccaccagcatgatgggatggaggcctatgtgaaggtggacagctgcccagaagagccccagctgcagatgaagaacaatgaggaggcagaggactatgatgatgacctgactgactctgagatggatgttgtgagatttgatgatgacaacagccccagcttcatccagatcagaagtgttgccaagaagcaccccaagacctgggtgcactacattgcagctgaggaggaggactgggactatgcccccctggtgctggccccagatgacagaagctacaagagccagtacctgaacaatggcccccagagaattggtagaaagtacaagaaggtgagattcatggcctacacagatgagaccttcaagaccagagaggccatccagcatgagtcaggaatcctgggccccctgctgtatggggaggtgggggataccctgctgatcatcttcaagaaccaggccagcagaccctacaacatctacccccatggcatcacagatgtgagacccctgtacagcagaagactgcccaagggagtgaagcacctgaaggacttccccatcctgcctggtgagatcttcaagtacaagtggacagttactgtggaagatggtcccaccaagtcagaccccagatgcctgaccagatactacagcagctttgtgaacatggagagagacctggcctctgggctgattgggcccctgctgatctgctacaaggagagtgtggaccagcagggcaaccagatcatgtctgataagagaaatgtgatcctgttctctgtatttgatgaaaacagaagctggtacctgactgagaacatccagagattcctgcccaacccagctggagtccagctggaggacccagagttccaggccagcaacatcatgcacagcatcaatggatatgtgtttgacagcctgcagctgtctgtatgcctgcatgaggtggcctactggtacatcctgagcattggggcccagactgacttcctgtctgtgttcttctcaggatacaccttcaagcacaagatggtgtatgaggacaccctgaccctgttccccttctcaggggaaactgtgttcatgagcatggagaaccctggcctgtggatcctgggctgccacaactctgacttcagaaacagaggcatgactgccctgctgaaggtgagcagctgtgacaagaacactggggattactatgaggacagctatgaggacatctcagcctacctgctgagcaagaacaatgccattgaacccagaagcttcagccagaacccccctgtcctgaagagacaccagagagagatcaccagaaccaccctgcagtctgatcaggaggagattgactatgatgataccatctcagtggagatgaagaaggaggactttgacatctatgatgaagatgaaaaccagagccccagaagcttccagaagaagaccagacactacttcattgctgctgtggagagactgtgggactatgggatgagcagcagcccccatgtgctgagaaacagagcccagtcaggcagtgtgccccagttcaagaaggtggtgttccaggagttcacagatggaagcttcacccagcccctgtacagaggtgagctgaatgaacacctgggcctgctgggcccctacatcagagctgaagtggaggacaacatcatggtgaccttcagaaaccaggccagcagaccctacagcttctacagcagcctgatcagctatgaggaggaccagagacagggagcagagcccagaaagaactttgtcaagcccaatgaaaccaagacctacttctggaaggtgcagcaccacatggcccccaccaaggatgaatttgactgcaaggcctgggcctacttctctgatgtagacctggagaaggatgtgcactctgggctgattggccccctgctggtgtgccacaccaacaccctgaacccagcccatggcagacaggtgactgtgcaggagtttgccctgttcttcaccatctttgatgaaaccaagagctggtacttcactgagaacatggagagaaactgcagagccccctgcaacatccagatggaggaccccaccttcaaggagaactacagattccatgctatcaatggatacatcatggacaccctgcctgggctggtgatggcccaggaccagagaatcagatggtacctgctgagcatgggcagcaatgagaacatccacagcatccacttctcaggacatgtcttcacagtaagaaagaaggaggagtacaagatggccctgtacaacctgtacccaggggtctttgaaactgtagagatgctgcccagcaaggcaggcatctggagagtggagtgcctgattggagagcacctgcatgctggtatgagcaccctgttcctggtgtacagcaacaagtgccagacccccctgggcatggcctctggccacatcagagacttccagatcacagccagtggccagtatgggcagtgggcccccaagctggccagactgcactactctggcagcatcaatgcctggagcaccaaggagcccttcagctggatcaaggtggacctgctggcccccatgatcatccatgggatcaagacccagggtgccagacagaagttcagcagcctgtacatcagccagttcatcatcatgtacagcctggatggcaagaagtggcagacctacagaggcaacagcacaggtaccctgatggtgttctttggcaatgtggacagctcaggcatcaagcacaacatcttcaacccccccatcattgccagatacatcagactgcaccccacccactacagcatcagaagcaccctgagaatggagctgatgggctgtgacctgaacagctgcagcatgcccctgggcatggagagcaaggccatctctgatgcccagatcacagctagcagctacttcaccaacatgtttgccacctggagccccagcaaggccagactgcacctgcagggcagaagcaatgcttggagaccccaggtgaacaaccccaaggagtggctgcaggtggacttccagaagaccatgaaggtgacaggtgtgaccacccagggggttaagagcctgctgaccagcatgtatgtgaaggagttcctgatcagcagcagccaggatggccaccagtggaccctgttcttccagaatggcaaggtgaaggtgttccagggcaaccaggacagcttcacccctgttgtgaacagcctggacccccccctgctgaccagatacctgagaatccacccccagagctgggtgcaccagattgccctgagaatggaggtgctgggctgtgaggcccaggacctgtactga

SEQ ID NO: 6 - DNA, 4374 bp, synthetic construct
  source 1..4374: mol_type = other DNA
atgcagatagaactgagcacctgcttcttcctgtgcctgctgagattctgcttctcagccaccagaagatactacctgggggcagttgagctgagctgggactacatgcagagtgatctgggggagctgccagtggatgcaagattcccccccagagtgcccaagagcttccccttcaacaccagtgtggtctacaagaagaccctgtttgttgagttcacagaccacctgttcaacattgctaagcccagacccccctggatgggcctgctgggccccaccatccaggcagaggtgtatgatacagttgtgatcaccctgaagaacatggccagccacccagttagcctgcatgcagttggagtgagctactggaaggcctcagagggagctgagtatgatgaccagaccagccagagagagaaggaggatgataaggtgttcccaggggggagccacacctatgtgtggcaggtgctgaaggagaatggacccatggccagtgaccccctgtgcctgacctacagctacctgagccatgtggacctggtgaaggacctgaactcaggactgataggtgctctgctggtgtgcagagagggcagcctggccaaggagaagacccagaccctgcacaagttcatcctgctgtttgcagtttttgatgaaggcaagagctggcactcagagaccaagaacagcctgatgcaggacagagatgcagcttctgctagagcctggcccaagatgcacactgtcaatggctatgtaaacagaagcctgcctggcctgattggctgccacagaaagtctgtctactggcatgttataggtatgggcaccacccctgaggtgcacagcatcttcctggagggccacaccttcctggtgagaaaccacagacaggccagcctggagatcagccccatcaccttcctgactgcccagaccctgctgatggacctgggccagttcctgctgttctgccacatcagcagccaccagcatgatggcatggaggcctatgtcaaggtggacagctgcccagaagagccccagctgcagatgaagaacaatgaggaggctgaggactatgatgatgacctgactgacagtgagatggatgtagtgagatttgatgatgacaacagccccagcttcatccagatcagaagtgtagccaagaagcaccccaagacctgggtgcactacattgctgcagaggaggaggactgggactatgcccccctggtgctggccccagatgacagaagctacaagagccagtacctgaacaatggtccccagagaattggcagaaagtacaagaaggtgagattcatggcctacacagatgagaccttcaagaccagagaggccatccagcatgagtcaggcatcctgggccccctgctgtatggagaggtgggggacaccctgctgatcatcttcaagaaccaggccagcagaccctacaacatctacccccatggaatcacagatgtgagacccctgtacagcagaagactgcccaagggggtgaagcacctgaaggacttccccatcctgcctggagagatcttcaagtacaagtggactgtgacagtagaggatggacccaccaagtcagaccccagatgcctgaccagatactacagcagctttgtaaacatggagagagacctggccagtggcctgataggccccctgctgatctgctacaaggagtcagtggaccagcagggcaaccagatcatgtcagacaagagaaatgtgatcctgttctctgtatttgatgagaacagaagctggtacctgactgaaaacatccagagattcctgcccaaccctgctggagtacagctggaggaccctgagttccaggccagcaacatcatgcacagcatcaatggttatgtgtttgacagcctgcagctgtctgtgtgcctgcatgaagtggcctactggtacatcctgagcataggtgcccagactgacttcctgtctgtgttcttcagtggctacaccttcaagcacaagatggtgtatgaagacaccctgaccctgttccccttctctggggagacagtattcatgagcatggagaacccaggactgtggatcctgggctgccacaacagtgacttcagaaacagaggcatgactgctctgctgaaggtgagcagctgtgataagaacacaggtgactactatgaggacagctatgaagacatcagtgcttacctgctgagcaagaacaatgctatagaacccagaagcttcagccagaaccccccagttctgaagagacaccagagagagatcaccagaaccaccctgcagtcagaccaggaggagatagactatgatgataccatctcagtagagatgaagaaggaggactttgacatctatgatgaggatgaaaaccagagccccagaagcttccagaagaagaccagacactacttcatagctgctgtagagagactgtgggactatggaatgagcagcagcccccatgtgctgagaaacagagcccagtctgggagtgtgccccagttcaagaaggtggtgttccaggagttcacagatgggagcttcacccagcccctgtacagaggggagctgaatgaacacctgggcctgctgggcccctacatcagagcagaggtggaggacaacatcatggtgaccttcagaaaccaggccagcagaccctacagcttctacagcagcctgatcagctatgaggaggaccagagacagggagctgagcccagaaagaactttgttaagcccaatgaaaccaagacctacttctggaaggtgcagcaccacatggcccccaccaaggatgagtttgactgcaaggcctgggcctacttctctgatgttgacctggagaaggatgtgcactctggactgattgggcccctgctggtgtgccacaccaacaccctgaaccctgcccatgggagacaggtgacagtacaggagtttgctctgttcttcaccatctttgatgagaccaagagctggtacttcacagaaaacatggagagaaactgcagagccccctgcaacatccagatggaggaccccaccttcaaggagaactacagattccatgctatcaatggctacatcatggacaccctgcctggcctggtgatggcccaggaccagagaatcagatggtacctgctgagcatgggcagcaatgaaaacatccacagcatccacttctctggtcatgttttcacagttagaaagaaggaggagtacaagatggccctgtacaacctgtaccctggggtgtttgagactgtggagatgctgcccagcaaggctggaatctggagagtggagtgcctgattggagaacacctgcatgcagggatgagcaccctgttcctggtgtacagcaacaagtgccagacccccctgggcatggccagtggtcacatcagagacttccagatcacagcctcaggccagtatggacagtgggcccccaagctggccagactgcactacagtggtagcatcaatgcctggagcaccaaggagcccttcagctggatcaaggtggacctgctggcccccatgatcatccatggaatcaagacccagggggccagacagaagttcagcagcctgtacatcagccagttcatcatcatgtacagcctggatggcaagaagtggcagacctacagaggcaacagcactggtaccctgatggtgttctttggcaatgtggacagctctgggatcaagcacaacatcttcaacccccccatcatagccagatacatcagactgcaccccacccactacagcatcagaagcaccctgagaatggagctgatgggctgtgacctgaacagctgcagcatgcccctgggcatggagagcaaggccatcagtgatgctcagatcacagcaagcagctacttcaccaacatgtttgccacctggagccccagcaaggccagactgcacctgcagggcagaagcaatgcttggagaccccaggtgaacaaccccaaggagtggctgcaggtggacttccagaagaccatgaaggtgactggggtgaccacccagggggtgaagagcctgctgaccagcatgtatgtcaaggagttcctgatcagcagcagccaggatgggcaccagtggaccctgttcttccagaatgggaaggtgaaggtgttccagggcaaccaggacagcttcaccccagtagtgaacagcctggacccccccctgctgaccagatacctgagaatccacccccagagctgggtgcaccagatagccctgagaatggaggtgctgggctgtgaggcccaggacctgtactga

SEQ ID NO: 7 - DNA, 4374 bp, synthetic construct
  source 1..4374: mol_type = other DNA
atgcagatagagctgagcacctgcttcttcctgtgcctgctgagattctgcttctcagccaccagaagatactacctgggtgcagtggagctgagctgggactacatgcagagtgacctgggggaactgcctgtggatgccagattcccccccagagtgcccaagagcttccccttcaacacctctgttgtttacaagaagaccctgtttgtagagttcactgaccacctgttcaacatagccaagcccagacccccctggatgggcctgctgggccccaccatccaggcagaggtgtatgatacagtggtgatcaccctgaagaacatggccagccaccctgttagcctgcatgcagtgggggtgagctactggaaggccagtgagggagcagagtatgatgatcagaccagccagagagagaaggaggatgacaaggtgttccctgggggcagccacacctatgtatggcaggtgctgaaggagaatggccccatggcctcagatcccctgtgcctgacctacagctacctgagccatgtggacctggtgaaggacctgaacagtggtctgattggggccctgctggtgtgcagagagggcagcctggccaaggagaagacccagaccctgcacaagttcatcctgctgtttgctgtctttgatgagggcaagagctggcactcagagaccaagaacagcctgatgcaggacagagatgctgcctctgccagagcctggcccaagatgcacactgtgaatgggtatgtgaacagaagcctgccaggtctgataggctgccacagaaagtcagtatactggcatgtgattggcatgggcaccaccccagaggtgcacagcatcttcctggagggccacaccttcctggtgagaaaccacagacaggccagcctggagatcagccccatcaccttcctgactgcccagaccctgctgatggacctgggccagttcctgctgttctgccacatcagcagccaccagcatgatggcatggaggcctatgttaaggtggacagctgcccagaggagccccagctgcagatgaagaacaatgaagaggctgaggactatgatgatgatctgacagactcagaaatggatgtagtcagatttgatgatgataacagccccagcttcatccagatcagatctgtagccaagaagcaccccaagacctgggtgcactacatagcagcagaggaggaggactgggactatgcacccctggtgctggcccctgatgacagaagctacaagagccagtacctgaacaatggcccccagagaataggcagaaagtacaagaaggtgagattcatggcctacactgatgagaccttcaagaccagagaggccatccagcatgagtctggaatcctgggccccctgctgtatggtgaggtgggtgacaccctgctgatcatcttcaagaaccaggccagcagaccctacaacatctacccccatggcatcactgatgtgagacccctgtacagcagaagactgcccaagggggtgaagcacctgaaggacttccccatcctgccaggtgagatcttcaagtacaagtggactgtgacagtggaggatggccccaccaagagtgaccccagatgcctgaccagatactacagcagctttgtcaacatggagagagacctggccagtgggctgatagggcccctgctgatctgctacaaggagtcagtggaccagcagggcaaccagatcatgagtgataagagaaatgtgatcctgttctcagtttttgatgaaaacagaagctggtacctgacagaaaacatccagagattcctgcccaacccagcaggggtacagctggaggaccctgaattccaggccagcaacatcatgcacagcatcaatggctatgtatttgacagcctgcagctgtctgtttgcctgcatgaggtggcctactggtacatcctgagcataggagcccagacagacttcctgagtgtgttcttcagtggctacaccttcaagcacaagatggtgtatgaggacaccctgaccctgttccccttctcaggggagactgttttcatgagcatggagaaccctggactgtggatcctgggctgccacaactctgacttcagaaacagaggcatgactgccctgctgaaggtgagcagctgtgataagaacactggagactactatgaggacagctatgaggacatctcagcctacctgctgagcaagaacaatgccattgaacccagaagcttcagccagaacccccctgtactgaagagacaccagagagagatcaccagaaccaccctgcagagtgaccaggaggagatagactatgatgataccatctctgtggagatgaagaaggaggactttgatatctatgatgaggatgagaaccagagccccagaagcttccagaagaagaccagacactacttcatagcagcagtggagagactgtgggactatggcatgagcagcagcccccatgtgctgagaaacagagcccagtcaggcagtgttccccagttcaagaaggtggtgttccaggagttcactgatggtagcttcacccagcccctgtacagaggagagctgaatgagcacctgggcctgctgggcccctacatcagagctgaggtggaggacaacatcatggtgaccttcagaaaccaggccagcagaccctacagcttctacagcagcctgatcagctatgaggaggaccagagacagggagcagagcccagaaagaactttgttaagcccaatgaaaccaagacctacttctggaaggtgcagcaccacatggcccccaccaaggatgagtttgattgcaaggcctgggcctacttcagtgatgtagacctggagaaggatgtgcactcaggcctgattggacccctgctggtgtgccacaccaacaccctgaaccctgcacatggaagacaggtgacagtgcaggagtttgccctgttcttcaccatctttgatgagaccaagagctggtacttcacagagaacatggagagaaactgcagagccccctgcaacatccagatggaggaccccaccttcaaggagaactacagattccatgctatcaatggctacatcatggacaccctgcctggcctggtgatggcccaggaccagagaatcagatggtacctgctgagcatgggcagcaatgagaacatccacagcatccacttcagtgggcatgtgttcacagtgagaaagaaggaggagtacaagatggccctgtacaacctgtaccctggagtgtttgagactgtggagatgctgcccagcaaggcaggcatctggagagtggagtgcctgataggagaacacctgcatgctggcatgagcaccctgttcctggtgtacagcaacaagtgccagacccccctgggcatggccagtggacacatcagagacttccagatcacagcctctggacagtatggacagtgggcccccaagctggccagactgcactactcaggaagcatcaatgcatggagcaccaaggagcccttcagctggatcaaggtggacctgctggcccccatgatcatccatggcatcaagacccagggggcaagacagaagttcagcagcctgtacatcagccagttcatcatcatgtacagcctggatggcaagaagtggcagacctacagaggcaacagcactggaaccctgatggtgttctttgggaatgtggacagctcagggatcaagcacaacatcttcaacccccccatcattgctagatacatcagactgcaccccacccactacagcatcagaagcaccctgagaatggagctgatgggctgtgatctgaacagctgcagcatgcccctgggcatggagagcaaggccatctcagatgcccagatcactgccagcagctacttcaccaacatgtttgccacctggagccccagcaaggccagactgcacctgcagggcagaagcaatgcttggagaccccaggtgaacaaccccaaggagtggctgcaggtggacttccagaagaccatgaaggtgactggagtaaccacccagggggtgaagagcctgctgaccagcatgtatgtgaaggagttcctgatcagcagcagccaggatggtcaccagtggaccctgttcttccagaatggcaaggtgaaggtgttccagggcaaccaggacagcttcacccctgttgtgaacagcctggacccccccctgctgaccagatacctgagaatccacccccagagctgggtgcaccagattgccctgagaatggaggtgctgggctgtgaggcccaggacctgtactga

SEQ ID NO: 8 - DNA, 4374 bp, synthetic construct
  source 1..4374: mol_type = other DNA
atgcagatagagctgagcacctgcttcttcctgtgcctgctgagattctgcttctctgccaccagaagatactacctgggtgcagtggagctgagctgggactacatgcagtcagacctgggagagctgccagtggatgctagattcccccccagagtgcccaagagcttccccttcaacacctcagtggtgtacaagaagaccctgtttgtagagttcactgaccacctgttcaacattgccaagcccagacccccctggatgggcctgctgggccccaccatccaggctgaggtgtatgatacagtggtaatcaccctgaagaacatggccagccacccagtgagcctgcatgctgttggagttagctactggaaggcctctgagggtgctgagtatgatgatcagaccagccagagagagaaggaggatgataaggtgttcccagggggaagccacacctatgtatggcaggtgctgaaggagaatgggcccatggccagtgaccccctgtgcctgacctacagctacctgagccatgtggatctggtgaaggacctgaactcaggtctgataggggccctgctggtgtgcagagagggcagcctggccaaggagaagacccagaccctgcacaagttcatcctgctgtttgctgtatttgatgagggcaagagctggcacagtgaaaccaagaacagcctgatgcaggacagagatgctgcctctgccagagcctggcccaagatgcacacagtgaatggctatgtgaacagaagcctgcctggtctgatagggtgccacagaaagtcagtgtactggcatgtcattggcatgggcaccaccccagaggtgcacagcatcttcctggagggccacaccttcctggtgagaaaccacagacaggccagcctggagatcagccccatcaccttcctgacagctcagaccctgctgatggacctgggccagttcctgctgttctgccacatcagcagccaccagcatgatggcatggaggcctatgttaaggtggacagctgcccagaggagccccagctgcagatgaagaacaatgaggaggcagaggactatgatgatgatctgacagacagtgagatggatgtggtgagatttgatgatgacaacagccccagcttcatccagatcagatctgtggccaagaagcaccccaagacctgggtgcactacattgcagctgaagaggaggactgggactatgcccccctggtgctggccccagatgatagaagctacaagagccagtacctgaacaatggcccccagagaattggaagaaagtacaagaaggtgagattcatggcctacactgatgaaaccttcaagaccagagaggccatccagcatgagtcaggcatcctgggccccctgctgtatggtgaggtgggtgataccctgctgatcatcttcaagaaccaggccagcagaccctacaacatctacccccatggcatcactgatgtaagacccctgtacagcagaagactgcccaagggtgtaaagcacctgaaggacttccccatcctgcctggggaaatcttcaagtacaagtggactgtgactgtagaggatggtcccaccaagagtgaccccagatgcctgaccagatactacagcagctttgtgaacatggagagagacctggcctctggtctgataggtcccctgctgatctgctacaaggagtcagtagaccagcagggcaaccagatcatgtctgacaagagaaatgtgatcctgttcagtgtgtttgatgagaacagaagctggtacctgacagaaaacatccagagattcctgcccaacccagctggagtgcagctggaggaccctgagttccaggccagcaacatcatgcacagcatcaatgggtatgtctttgacagcctgcagctgagtgtgtgcctgcatgaggtggcctactggtacatcctgagcattggtgctcagacagatttcctgtctgtgttcttcagtggttacaccttcaagcacaagatggtgtatgaagacaccctgaccctgttccccttcagtggagagacagtattcatgagcatggagaaccctggcctgtggatcctgggctgccacaacagtgatttcagaaacagaggcatgactgcactgctgaaggtgagcagctgtgacaagaacactggggattactatgaagacagctatgaggacatcagtgcctacctgctgagcaagaacaatgctattgagcccagaagcttcagccagaaccccccagtcctgaagagacaccagagagagatcaccagaaccaccctgcagagtgaccaggaggagatagactatgatgacaccatctcagtggagatgaagaaggaggactttgacatctatgatgaggatgagaaccagagccccagaagcttccagaagaagaccagacactacttcattgcagcagtagagagactgtgggactatgggatgagcagcagcccccatgtactgagaaacagagcccagtctggatctgtgccccagttcaagaaggtggtgttccaggagttcactgatggcagcttcacccagcccctgtacagaggagagctgaatgagcacctgggcctgctgggcccctacatcagagcagaggtggaggacaacatcatggtgaccttcagaaaccaggccagcagaccctacagcttctacagcagcctgatcagctatgaggaggaccagagacagggtgctgagcccagaaagaactttgttaagcccaatgagaccaagacctacttctggaaggtgcagcaccacatggcccccaccaaggatgaatttgattgcaaggcctgggcctacttcagtgatgtagacctggagaaggatgtgcacagtggactgattgggcccctgctggtgtgccacaccaacaccctgaacccagcccatggcagacaggtgactgtgcaggagtttgctctgttcttcaccatctttgatgaaaccaagagctggtacttcacagagaacatggagagaaactgcagagccccctgcaacatccagatggaggaccccaccttcaaggagaactacagattccatgctatcaatggctacatcatggacaccctgccaggtctggtgatggcccaggaccagagaatcagatggtacctgctgagcatgggcagcaatgagaacatccacagcatccacttctctggccatgtcttcactgtgagaaagaaggaggagtacaagatggccctgtacaacctgtacccaggtgtctttgagactgtagagatgctgcccagcaaggctggcatctggagagtggagtgcctgattggagagcacctgcatgcaggaatgagcaccctgttcctggtgtacagcaacaagtgccagacccccctgggcatggccagtggccacatcagagacttccagatcacagcctctgggcagtatggacagtgggcccccaagctggccagactgcactactcaggaagcatcaatgcctggagcaccaaggagcccttcagctggatcaaggtggacctgctggcccccatgatcatccatgggatcaagacccagggggccagacagaagttcagcagcctgtacatcagccagttcatcatcatgtacagcctggatggtaagaagtggcagacctacagaggcaacagcacaggcaccctgatggtgttctttggaaatgtagacagctctggcatcaagcacaacatcttcaacccccccatcatagccagatacatcagactgcaccccacccactacagcatcagaagcaccctgagaatggagctgatgggctgtgacctgaacagctgcagcatgcccctgggcatggagagcaaggccatctctgatgctcagatcactgccagcagctacttcaccaacatgtttgcaacctggagccccagcaaggccagactgcacctgcagggcagaagcaatgcctggagaccccaggtgaacaaccccaaggagtggctgcaggtggacttccagaagaccatgaaggtgactggtgtaaccacccagggagtgaagagcctgctgaccagcatgtatgtcaaggagttcctgatcagcagcagccaggatggtcaccagtggaccctgttcttccagaatggaaaggtgaaggtgttccagggcaaccaggacagcttcaccccagttgtgaacagcctggacccccccctgctgaccagatacctgagaatccacccccagagctgggtgcaccagattgccctgagaatggaggtgctgggctgtgaggcccaggacctgtactga

SEQ ID NO: 9 - DNA, 4374 bp, synthetic construct
  source 1..4374: mol_type = other DNA
atgcagatagagctgagcacctgcttcttcctgtgcctgctgagattctgcttcagtgcaaccagaagatactacctgggtgcagtggagctgagctgggactacatgcagtctgatctgggggagctgccagttgatgccagattcccccccagagtgcccaagagcttccccttcaacaccagtgttgtttacaagaagaccctgtttgtggaattcactgaccacctgttcaacatagccaagcccagacccccctggatgggcctgctgggccccaccatccaggcagaggtgtatgacacagtagtgatcaccctgaagaacatggccagccaccctgtgagcctgcatgcagtaggggtaagctactggaaggcctctgaaggtgctgaatatgatgaccagaccagccagagagagaaggaggatgacaaggtgttcccaggtggaagccacacctatgtctggcaggtgctgaaggagaatggccccatggccagtgaccccctgtgcctgacctacagctacctgagccatgtggacctggtgaaggacctgaactcaggtctgattggagcactgctggtgtgcagagagggcagcctggccaaggagaagacccagaccctgcacaagttcatcctgctgtttgcagtgtttgatgaaggcaagagctggcacagtgagaccaagaacagcctgatgcaggacagagatgcagccagtgccagagcctggcccaagatgcacactgtgaatggttatgtcaacagaagcctgccagggctgataggctgccacagaaagagtgtttactggcatgtaattgggatgggcaccaccccagaggtgcacagcatcttcctggagggccacaccttcctggtgagaaaccacagacaggccagcctggagatcagccccatcaccttcctgacagctcagaccctgctgatggacctgggccagttcctgctgttctgccacatcagcagccaccagcatgatggcatggaggcctatgtaaaggtggacagctgccctgaggagccccagctgcagatgaagaacaatgaggaggcagaggactatgatgatgacctgactgacagtgagatggatgtggtgagatttgatgatgacaacagccccagcttcatccagatcagaagtgttgctaagaagcaccccaagacctgggtgcactacattgcagcagaggaggaggactgggactatgctcccctggtgctggcccctgatgacagaagctacaagagccagtacctgaacaatggcccccagagaattggcagaaagtacaagaaggtgagattcatggcctacactgatgagaccttcaagaccagagaggccatccagcatgaaagtggcatcctgggccccctgctgtatggagaggtgggagacaccctgctgatcatcttcaagaaccaggccagcagaccctacaacatctacccccatggtatcacagatgtgagacccctgtacagcagaagactgcccaagggggtgaagcacctgaaggacttccccatcctgccaggtgaaatcttcaagtacaagtggacagtcacagtggaggatggccccaccaagagtgaccccagatgcctgaccagatactacagcagctttgtgaacatggagagagacctggcctctggcctgataggccccctgctgatctgctacaaggagtcagtggaccagcagggcaaccagatcatgagtgataagagaaatgtgatcctgttctcagtatttgatgagaacagaagctggtacctgactgaaaacatccagagattcctgcccaaccctgcaggggtgcagctggaggaccctgaattccaggccagcaacatcatgcacagcatcaatggctatgtatttgatagcctgcagctgagtgtgtgcctgcatgaggtggcctactggtacatcctgagcataggggcccagacagacttcctgtctgtgttcttctcaggttacaccttcaagcacaagatggtgtatgaagacaccctgaccctgttccccttcagtggggaaacagtattcatgagcatggagaaccctgggctgtggatcctgggctgccacaactcagatttcagaaacagaggcatgactgccctgctgaaggtgagcagctgtgacaagaacactggagactactatgaggacagctatgaggacatcagtgcctacctgctgagcaagaacaatgctatagagcccagaagcttcagccagaaccccccagtcctgaagagacaccagagagagatcaccagaaccaccctgcagtcagatcaggaggagatagactatgatgacaccatcagtgtggagatgaagaaggaggactttgacatctatgatgaggatgagaaccagagccccagaagcttccagaagaagaccagacactacttcatagctgctgtagagagactgtgggactatggcatgagcagcagcccccatgtcctgagaaacagagcccagtctggctctgttccccagttcaagaaggtggtgttccaggagttcacagatggcagcttcacccagcccctgtacagaggggagctgaatgaacacctgggcctgctgggcccctacatcagagctgaagtggaggacaacatcatggtgaccttcagaaaccaggccagcagaccctacagcttctacagcagcctgatcagctatgaggaggaccagagacagggtgcagaacccagaaagaactttgttaagcccaatgagaccaagacctacttctggaaggtgcagcaccacatggcccccaccaaggatgaatttgactgcaaggcctgggcctacttctcagatgttgacctggagaaggatgtccactcaggactgatagggcccctgctggtgtgccacaccaacaccctgaacccagcccatggcagacaggtgactgtgcaggagtttgctctgttcttcaccatctttgatgagaccaagagctggtacttcacagagaacatggagagaaactgcagagccccctgcaacatccagatggaggaccccaccttcaaggagaactacagattccatgctatcaatggatacatcatggacaccctgccaggcctggtgatggcccaggaccagagaatcagatggtacctgctgagcatgggcagcaatgagaacatccacagcatccacttcagtggtcatgtgttcacagtaagaaagaaggaggagtacaagatggccctgtacaacctgtacccaggggtgtttgaaactgtggaaatgctgcccagcaaggcagggatctggagagtggagtgcctgattggagaacacctgcatgcaggcatgagcaccctgttcctggtgtacagcaacaagtgccagacccccctgggcatggccagtgggcacatcagagacttccagatcacagcctctgggcagtatggccagtgggcccccaagctggccagactgcactactcaggcagcatcaatgcctggagcaccaaggagcccttcagctggatcaaggtggacctgctggcccccatgatcatccatggcatcaagacccagggagcaagacagaagttcagcagcctgtacatcagccagttcatcatcatgtacagcctggatgggaagaagtggcagacctacagaggcaacagcactgggaccctgatggtgttctttggcaatgtagacagctctggcatcaagcacaacatcttcaacccccccatcattgcaagatacatcagactgcaccccacccactacagcatcagaagcaccctgagaatggagctgatgggctgtgacctgaacagctgcagcatgcccctgggcatggagagcaaggccatcagtgatgcccagatcactgctagcagctacttcaccaacatgtttgccacctggagccccagcaaggccagactgcacctgcagggcagaagcaatgcatggagaccccaggtgaacaaccccaaggagtggctgcaggtggacttccagaagaccatgaaggtgacaggggttaccacccagggtgtgaagagcctgctgaccagcatgtatgtgaaggagttcctgatcagcagcagccaggatggccaccagtggaccctgttcttccagaatgggaaggtgaaggtgttccagggcaaccaggacagcttcacccctgtggtgaacagcctggacccccccctgctgaccagatacctgagaatccacccccagagctgggtgcaccagattgccctgagaatggaggtgctgggctgtgaggcccaggacctgtactga

SEQ ID NO: 10 - DNA, 4374 bp, synthetic construct
  source 1..4374: mol_type = other DNA
atgcagatagaactgagcacctgcttcttcctgtgcctgctgagattctgcttctctgccaccagaagatactacctgggggctgtggagctgagctgggactacatgcagagtgacctgggagagctgccagttgatgcaagattcccccccagagtgcccaagagcttccccttcaacacctctgtggtgtacaagaagaccctgtttgtggagttcactgatcacctgttcaacatagccaagcccagacccccctggatgggcctgctgggccccaccatccaggctgaagtgtatgacactgtagtgatcaccctgaagaacatggccagccaccctgtgagcctgcatgctgtgggagtgagctactggaaggcctcagagggagcagagtatgatgaccagaccagccagagagagaaggaggatgacaaggtgttcccaggaggcagccacacctatgtttggcaggtgctgaaggagaatgggcccatggcctcagaccccctgtgcctgacctacagctacctgagccatgtagacctggtgaaggacctgaactctggcctgataggagccctgctggtgtgcagagagggcagcctggccaaggagaagacccagaccctgcacaagttcatcctgctgtttgcagtgtttgatgagggcaagagctggcactcagagaccaagaacagcctgatgcaggacagagatgcagccagtgctagagcctggcccaagatgcacacagtgaatgggtatgttaacagaagcctgccaggcctgataggatgccacagaaagtctgtgtactggcatgtgattggtatgggcaccaccccagaggtgcacagcatcttcctggagggccacaccttcctggtgagaaaccacagacaggccagcctggagatcagccccatcaccttcctgacagcccagaccctgctgatggacctgggccagttcctgctgttctgccacatcagcagccaccagcatgatggcatggaggcctatgttaaggtggacagctgcccagaggagccccagctgcagatgaagaacaatgaagaggcagaggactatgatgatgacctgacagattcagagatggatgtggtgagatttgatgatgacaacagccccagcttcatccagatcagaagtgtggccaagaagcaccccaagacctgggtgcactacattgctgctgaagaggaggactgggactatgcccccctggtgctggccccagatgacagaagctacaagagccagtacctgaacaatgggccccagagaattggaagaaagtacaagaaggtgagattcatggcctacacagatgaaaccttcaagaccagagaggccatccagcatgagagtgggatcctgggccccctgctgtatggggaggtgggtgataccctgctgatcatcttcaagaaccaggccagcagaccctacaacatctacccccatggcatcacagatgtgagacccctgtacagcagaagactgcccaagggagttaagcacctgaaggacttccccatcctgccaggagagatcttcaagtacaagtggacagtcacagtggaggatgggcccaccaagtctgaccccagatgcctgaccagatactacagcagctttgtaaacatggagagagacctggcctcaggtctgataggccccctgctgatctgctacaaggagagtgttgatcagcagggcaaccagatcatgtctgacaagagaaatgtgatcctgttctcagtgtttgatgaaaacagaagctggtacctgactgagaacatccagagattcctgcccaacccagctggggtgcagctggaggaccctgagttccaggccagcaacatcatgcacagcatcaatggatatgtgtttgacagcctgcagctgtcagtgtgcctgcatgaggtggcctactggtacatcctgagcataggagctcagacagacttcctgagtgtattcttctcagggtacaccttcaagcacaagatggtgtatgaggacaccctgaccctgttccccttctcaggtgagacagtgttcatgagcatggagaacccagggctgtggatcctgggctgccacaactctgatttcagaaacagaggcatgactgccctgctgaaggtgagcagctgtgacaagaacacaggggactactatgaggacagctatgaggacatctctgcctacctgctgagcaagaacaatgctattgagcccagaagcttcagccagaacccccctgtactgaagagacaccagagagagatcaccagaaccaccctgcagagtgatcaggaggagatagattatgatgacaccatctcagtggagatgaagaaggaggactttgatatctatgatgaggatgaaaaccagagccccagaagcttccagaagaagaccagacactacttcattgcagctgtagagagactgtgggactatggtatgagcagcagcccccatgtcctgagaaacagagcccagtctggatcagtcccccagttcaagaaggtggtgttccaggagttcacagatggtagcttcacccagcccctgtacagaggagagctgaatgagcacctgggcctgctgggcccctacatcagagcagaggtggaggacaacatcatggtgaccttcagaaaccaggccagcagaccctacagcttctacagcagcctgatcagctatgaagaggaccagagacagggagcagagcccagaaagaactttgtgaagcccaatgagaccaagacctacttctggaaggtgcagcaccacatggcccccaccaaggatgagtttgattgcaaggcctgggcctacttcagtgatgttgacctggagaaggatgttcacagtggcctgataggtcccctgctggtgtgccacaccaacaccctgaacccagcccatggcagacaggtgactgtccaggagtttgccctgttcttcaccatctttgatgagaccaagagctggtacttcactgaaaacatggagagaaactgcagagccccctgcaacatccagatggaggaccccaccttcaaggagaactacagattccatgccatcaatggctacatcatggacaccctgcctggactggtgatggcccaggaccagagaatcagatggtacctgctgagcatgggcagcaatgagaacatccacagcatccacttctcaggccatgtattcactgtgagaaagaaggaggagtacaagatggccctgtacaacctgtacccaggtgtgtttgagacagtggagatgctgcccagcaaggcaggtatctggagagtggagtgcctgataggagaacacctgcatgctggcatgagcaccctgttcctggtgtacagcaacaagtgccagacccccctgggcatggccagtggccacatcagagacttccagatcacagcctcaggccagtatgggcagtgggcccccaagctggccagactgcactactctggcagcatcaatgcatggagcaccaaggagcccttcagctggatcaaggtggacctgctggcccccatgatcatccatggaatcaagacccagggtgctagacagaagttcagcagcctgtacatcagccagttcatcatcatgtacagcctggatggcaagaagtggcagacctacagaggcaacagcactggcaccctgatggtgttctttggcaatgttgacagctctggcatcaagcacaacatcttcaacccccccatcatagcaagatacatcagactgcaccccacccactacagcatcagaagcaccctgagaatggagctgatgggctgtgatctgaacagctgcagcatgcccctgggcatggagagcaaggccatcagtgatgctcagatcactgccagcagctacttcaccaacatgtttgcaacctggagccccagcaaggccagactgcacctgcagggcagaagcaatgcctggagaccccaggtgaacaaccccaaggagtggctgcaggtggacttccagaagaccatgaaggtgacaggtgttaccacccagggtgtcaagagcctgctgaccagcatgtatgtaaaggagttcctgatcagcagcagccaggatggccaccagtggaccctgttcttccagaatggaaaggtgaaggtgttccagggcaaccaggacagcttcacccctgtggtgaacagcctggacccccccctgctgaccagatacctgagaatccacccccagagctgggtgcaccagatagccctgagaatggaggtgctgggctgtgaagcccaggacctgtactga

SEQ ID NO: 11 - DNA, 4374 bp, synthetic construct
  source 1..4374: mol_type = other DNA
atgcagatagagctgagcacctgcttcttcctgtgcctgctgagattctgcttctctgccaccagaagatactacctgggtgctgttgagctgagctgggactacatgcagtctgatctgggtgagctgcctgttgatgccagattcccccccagagtgcccaagagcttccccttcaacacctcagttgtgtacaagaagaccctgtttgttgagttcacagaccacctgttcaacattgctaagcccagacccccctggatgggcctgctgggccccaccatccaggcagaggtgtatgatacagttgtgatcaccctgaagaacatggccagccacccagtgagcctgcatgcagtaggtgtgagctactggaaggccagtgagggggctgaatatgatgatcagaccagccagagagagaaggaggatgacaaggtgttcccaggtggtagccacacctatgtatggcaggtgctgaaggagaatggccccatggccagtgaccccctgtgcctgacctacagctacctgagccatgtggacctggtgaaggacctgaactctggactgattggagcactgctggtgtgcagagagggcagcctggccaaggagaagacccagaccctgcacaagttcatcctgctgtttgcagtgtttgatgagggcaagagctggcactcagagaccaagaacagcctgatgcaggacagagatgcagcttctgccagagcctggcccaagatgcacactgtcaatggctatgtgaacagaagcctgccaggcctgattggttgccacagaaagagtgtgtactggcatgttatagggatgggcaccacccctgaggtgcacagcatcttcctggagggccacaccttcctggtgagaaaccacagacaggccagcctggagatcagccccatcaccttcctgacagcacagaccctgctgatggacctgggccagttcctgctgttctgccacatcagcagccaccagcatgatggcatggaggcctatgttaaggtggacagctgccctgaggagccccagctgcagatgaagaacaatgaggaggctgaggactatgatgatgacctgacagactcagagatggatgtggtgagatttgatgatgataacagccccagcttcatccagatcagatcagtggccaagaagcaccccaagacctgggtgcactacatagctgctgaagaggaggactgggactatgcccccctggtgctggccccagatgacagaagctacaagagccagtacctgaacaatgggccccagagaattggcagaaagtacaagaaggtgagattcatggcctacactgatgagaccttcaagaccagagaggccatccagcatgagtctggaatcctgggccccctgctgtatggagaggtgggtgacaccctgctgatcatcttcaagaaccaggccagcagaccctacaacatctacccccatggcatcactgatgttagacccctgtacagcagaagactgcccaagggtgtgaagcacctgaaggacttccccatcctgcctggggagatcttcaagtacaagtggacagtcactgtagaggatggccccaccaagagtgaccccagatgcctgaccagatactacagcagctttgtgaacatggagagagacctggcctctggactgattggacccctgctgatctgctacaaggagagtgtggaccagcagggcaaccagatcatgtcagataagagaaatgtcatcctgttctcagtatttgatgagaacagaagctggtacctgactgagaacatccagagattcctgcccaacccagctggggtgcagctggaggaccctgaattccaggccagcaacatcatgcacagcatcaatggctatgtatttgatagcctgcagctgtctgtgtgcctgcatgaggtggcctactggtacatcctgagcattggggcccagactgatttcctgtctgtcttcttctctgggtacaccttcaagcacaagatggtgtatgaggacaccctgaccctgttccccttctcaggagagacagtgttcatgagcatggagaacccaggcctgtggatcctgggctgccacaactcagacttcagaaacagaggcatgactgccctgctgaaggtgagcagctgtgacaagaacacaggtgactactatgaggacagctatgaagacatctctgcctacctgctgagcaagaacaatgctatagaacccagaagcttcagccagaacccccctgtgctgaagagacaccagagagagatcaccagaaccaccctgcagtcagatcaggaggagatagattatgatgataccatctcagtagaaatgaagaaggaggactttgacatctatgatgaagatgaaaaccagagccccagaagcttccagaagaagaccagacactacttcatagctgctgtagagagactgtgggactatggcatgagcagcagcccccatgtactgagaaacagagcccagagtggctcagtgccccagttcaagaaggtggtgttccaggagttcactgatggcagcttcacccagcccctgtacagaggtgaactgaatgagcacctgggcctgctgggcccctacatcagagcagaggtggaggacaacatcatggtgaccttcagaaaccaggccagcagaccctacagcttctacagcagcctgatcagctatgaagaggaccagagacagggtgctgagcccagaaagaactttgtgaagcccaatgagaccaagacctacttctggaaggtgcagcaccacatggcccccaccaaggatgagtttgactgcaaggcctgggcctacttcagtgatgtagacctggagaaggatgtgcacagtgggctgataggtcccctgctggtgtgccacaccaacaccctgaacccagcacatggtagacaggtgacagttcaggagtttgccctgttcttcaccatctttgatgaaaccaagagctggtacttcactgagaacatggagagaaactgcagagccccctgcaacatccagatggaggaccccaccttcaaggagaactacagattccatgccatcaatggctacatcatggacaccctgccaggcctggtgatggcccaggaccagagaatcagatggtacctgctgagcatgggcagcaatgagaacatccacagcatccacttctcaggacatgtgttcactgtgagaaagaaggaggagtacaagatggccctgtacaacctgtacccaggggtatttgaaacagtagagatgctgcccagcaaggcaggcatctggagagtggagtgcctgattggagaacacctgcatgcaggtatgagcaccctgttcctggtgtacagcaacaagtgccagacccccctgggcatggcctcaggccacatcagagacttccagatcacagcttctggccagtatgggcagtgggcccccaagctggccagactgcactactcaggtagcatcaatgcttggagcaccaaggagcccttcagctggatcaaggtggacctgctggcccccatgatcatccatgggatcaagacccagggagccagacagaagttcagcagcctgtacatcagccagttcatcatcatgtacagcctggatggtaagaagtggcagacctacagaggcaacagcactggcaccctgatggtgttctttggaaatgttgatagctcagggatcaagcacaacatcttcaacccccccatcatagccagatacatcagactgcaccccacccactacagcatcagaagcaccctgagaatggagctgatgggctgtgatctgaacagctgcagcatgcccctgggcatggagagcaaggccatctctgatgcacagatcacagcaagcagctacttcaccaacatgtttgccacctggagccccagcaaggccagactgcacctgcagggcagaagcaatgcttggagaccccaggtgaacaaccccaaggagtggctgcaggtggacttccagaagaccatgaaggtgacaggggtgaccacccagggggtgaagagcctgctgaccagcatgtatgtcaaggagttcctgatcagcagcagccaggatggccaccagtggaccctgttcttccagaatgggaaggtgaaggtgttccagggcaaccaggacagcttcaccccagtagtgaacagcctggacccccccctgctgaccagatacctgagaatccacccccagagctgggtgcaccagattgctctgagaatggaggtgctgggctgtgaggcccaggacctgtactga

SEQ ID NO: 12 - DNA, 4374 bp, synthetic construct
  source 1..4374: mol_type = other DNA
atgcagatagaactgagcacctgcttcttcctgtgcctgctgagattctgcttctctgccaccagaagatactacctgggggctgtggagctgagctgggactacatgcagtctgacctgggagaactgcctgttgatgctagattcccccccagagtgcccaagagcttccccttcaacacctcagtggtgtacaagaagaccctgtttgtagagttcactgaccacctgttcaacattgcaaagcccagacccccctggatgggcctgctgggccccaccatccaggctgaggtgtatgatactgttgtgatcaccctgaagaacatggccagccaccctgtgagcctgcatgctgtgggagtcagctactggaaggcctcagagggggctgagtatgatgaccagaccagccagagagagaaggaggatgacaaggtgttccctggtgggagccacacctatgtgtggcaggtgctgaaggagaatggacccatggcctcagaccccctgtgcctgacctacagctacctgagccatgtagacctggtgaaggacctgaacagtggtctgataggtgcactgctggtgtgcagagagggcagcctggccaaggagaagacccagaccctgcacaagttcatcctgctgtttgcagtgtttgatgagggcaagagctggcactctgagaccaagaacagcctgatgcaggacagagatgctgcctcagccagagcctggcccaagatgcacactgttaatgggtatgtgaacagaagcctgccaggcctgattggctgccacagaaagagtgtgtactggcatgtcataggaatgggcaccacccctgaggtgcacagcatcttcctggagggccacaccttcctggtgagaaaccacagacaggccagcctggagatcagccccatcaccttcctgactgcacagaccctgctgatggacctgggccagttcctgctgttctgccacatcagcagccaccagcatgatgggatggaggcctatgtaaaggtggacagctgccctgaagagccccagctgcagatgaagaacaatgaggaggcagaagactatgatgatgatctgacagacagtgaaatggatgtggtgagatttgatgatgacaacagccccagcttcatccagatcagaagtgtggccaagaagcaccccaagacctgggtgcactacattgctgcagaggaggaggactgggactatgcccccctggtgctggccccagatgacagaagctacaagagccagtacctgaacaatgggccccagagaatagggagaaagtacaagaaggtgagattcatggcctacacagatgagaccttcaagaccagagaggccatccagcatgagagtggtatcctgggccccctgctgtatggagaagtgggggataccctgctgatcatcttcaagaaccaggccagcagaccctacaacatctacccccatggcatcacagatgtgagacccctgtacagcagaagactgcccaagggtgtaaagcacctgaaggacttccccatcctgccaggggaaatcttcaagtacaagtggactgtgactgtggaggatggacccaccaagagtgatcccagatgcctgaccagatactacagcagctttgttaacatggagagagacctggcctcaggactgattggccccctgctgatctgctacaaggagagtgtggaccagcagggcaaccagatcatgagtgacaagagaaatgtcatcctgttcagtgtctttgatgaaaacagaagctggtacctgacagaaaacatccagagattcctgcccaaccctgcaggtgtgcagctggaggacccagagttccaggccagcaacatcatgcacagcatcaatgggtatgtgtttgatagcctgcagctgagtgtatgcctgcatgaggtggcctactggtacatcctgagcataggtgctcagactgacttcctgtcagtattcttctcaggctacaccttcaagcacaagatggtgtatgaggacaccctgaccctgttccccttcagtggggaaacagtgttcatgagcatggagaaccctgggctgtggatcctgggctgccacaacagtgacttcagaaacagaggcatgacagccctgctgaaggtgagcagctgtgacaagaacacaggagactactatgaagacagctatgaagacatcagtgcttacctgctgagcaagaacaatgcaatagagcccagaagcttcagccagaaccccccagtgctgaagagacaccagagagagatcaccagaaccaccctgcagtcagaccaggaggagattgactatgatgacaccatctctgtggagatgaagaaggaggactttgacatctatgatgaggatgagaaccagagccccagaagcttccagaagaagaccagacactacttcatagcagcagtggagagactgtgggactatgggatgagcagcagcccccatgtcctgagaaacagagcccagtcaggaagtgtcccccagttcaagaaggtggtgttccaggagttcactgatgggagcttcacccagcccctgtacagaggagagctgaatgaacacctgggcctgctgggcccctacatcagagcagaggtggaggacaacatcatggtgaccttcagaaaccaggccagcagaccctacagcttctacagcagcctgatcagctatgaggaggaccagagacagggagctgaacccagaaagaactttgtgaagcccaatgaaaccaagacctacttctggaaggtgcagcaccacatggcccccaccaaggatgagtttgactgcaaggcctgggcctacttctctgatgttgatctggagaaggatgtacactctgggctgattggccccctgctggtgtgccacaccaacaccctgaacccagcccatggcagacaggtgactgtgcaggagtttgccctgttcttcaccatctttgatgagaccaagagctggtacttcactgaaaacatggagagaaactgcagagccccctgcaacatccagatggaggaccccaccttcaaggagaactacagattccatgctatcaatggctacatcatggacaccctgcctggactggtgatggcccaggaccagagaatcagatggtacctgctgagcatgggcagcaatgaaaacatccacagcatccacttctcaggccatgtattcactgtgagaaagaaggaggagtacaagatggccctgtacaacctgtacccaggtgtgtttgaaacagtggaaatgctgcccagcaaggctggcatctggagagtggagtgcctgattggtgagcacctgcatgctggaatgagcaccctgttcctggtgtacagcaacaagtgccagacccccctgggcatggcctcagggcacatcagagacttccagatcactgcctctggccagtatggacagtgggcccccaagctggccagactgcactacagtggcagcatcaatgcctggagcaccaaggagcccttcagctggatcaaggtggacctgctggcccccatgatcatccatggcatcaagacccagggtgccagacagaagttcagcagcctgtacatcagccagttcatcatcatgtacagcctggatggcaagaagtggcagacctacagaggcaacagcacaggtaccctgatggtgttctttggcaatgtggacagctctggtatcaagcacaacatcttcaacccccccatcatagccagatacatcagactgcaccccacccactacagcatcagaagcaccctgagaatggagctgatgggctgtgatctgaacagctgcagcatgcccctgggcatggagagcaaggccatctctgatgctcagatcacagctagcagctacttcaccaacatgtttgccacctggagccccagcaaggccagactgcacctgcagggcagaagcaatgcatggagaccccaggtgaacaaccccaaggagtggctgcaggtggacttccagaagaccatgaaggtgacaggagtgaccacccagggagtgaagagcctgctgaccagcatgtatgtgaaggagttcctgatcagcagcagccaggatggccaccagtggaccctgttcttccagaatgggaaggtgaaggtgttccagggcaaccaggacagcttcaccccagtagtgaacagcctggacccccccctgctgaccagatacctgagaatccacccccagagctgggtgcaccagatagctctgagaatggaggtgctgggctgtgaagcccaggacctgtactga

SEQ ID NO: 13 - DNA, 4374 bp, synthetic construct
  source 1..4374: mol_type = other DNA
atgcagatagaactgagcacctgcttcttcctgtgcctgctgagattctgcttcagtgccaccagaagatactacctgggtgctgttgagctgagctgggactacatgcagtctgacctgggtgaactgccagtagatgcaagattcccccccagagtgcccaagagcttccccttcaacacctctgtagtctacaagaagaccctgtttgtggagttcacagatcacctgttcaacattgctaagcccagacccccctggatgggcctgctgggccccaccatccaggcagaggtgtatgacactgtagtgatcaccctgaagaacatggccagccacccagtgagcctgcatgctgtgggtgtgagctactggaaggccagtgaaggagcagagtatgatgatcagaccagccagagagagaaggaggatgacaaggtgttcccaggtggaagccacacctatgtctggcaggtgctgaaggagaatgggcccatggcctcagatcccctgtgcctgacctacagctacctgagccatgttgacctggtgaaggacctgaacagtggactgataggtgctctgctggtgtgcagagagggcagcctggccaaggagaagacccagaccctgcacaagttcatcctgctgtttgcagtgtttgatgaaggcaagagctggcacagtgagaccaagaacagcctgatgcaggacagagatgcagcatcagccagagcctggcccaagatgcacacagtgaatggctatgtgaacagaagcctgccaggcctgataggatgccacagaaagtctgtatactggcatgtcattggtatgggcaccacccctgaagtgcacagcatcttcctggagggccacaccttcctggtgagaaaccacagacaggccagcctggagatcagccccatcaccttcctgacagcccagaccctgctgatggacctgggccagttcctgctgttctgccacatcagcagccaccagcatgatgggatggaggcctatgtcaaggtggacagctgcccagaggagccccagctgcagatgaagaacaatgaggaggctgaggactatgatgatgacctgactgactcagagatggatgtggtgagatttgatgatgataacagccccagcttcatccagatcagatctgtggccaagaagcaccccaagacctgggtgcactacatagctgcagaagaggaggactgggactatgcccccctggtgctggccccagatgatagaagctacaagagccagtacctgaacaatggcccccagagaattgggagaaagtacaagaaggtgagattcatggcctacacagatgaaaccttcaagaccagagaggccatccagcatgagtctggcatcctgggccccctgctgtatggggaagtgggagacaccctgctgatcatcttcaagaaccaggccagcagaccctacaacatctacccccatggtatcacagatgtgagacccctgtacagcagaagactgcccaagggggtgaagcacctgaaggacttccccatcctgcctggggagatcttcaagtacaagtggacagtaactgtggaggatggccccaccaagtctgaccccagatgcctgaccagatactacagcagctttgtgaacatggagagagacctggccagtggcctgattggccccctgctgatctgctacaaggagagtgttgaccagcagggcaaccagatcatgagtgataagagaaatgttatcctgttcagtgtgtttgatgaaaacagaagctggtacctgacagagaacatccagagattcctgcccaacccagctggtgtccagctggaggaccctgagttccaggccagcaacatcatgcacagcatcaatggctatgtatttgacagcctgcagctgagtgtgtgcctgcatgaagtggcctactggtacatcctgagcataggtgctcagacagacttcctgagtgtcttcttcagtggctacaccttcaagcacaagatggtgtatgaagacaccctgaccctgttccccttcagtggagagacagtgttcatgagcatggagaacccaggactgtggatcctgggctgccacaacagtgacttcagaaacagaggcatgacagccctgctgaaggtgagcagctgtgacaagaacactggggactactatgaggacagctatgaggacatctctgcctacctgctgagcaagaacaatgcaatagagcccagaagcttcagccagaacccccctgtgctgaagagacaccagagagagatcaccagaaccaccctgcagtcagatcaggaggagatagactatgatgataccatctctgtggagatgaagaaggaggactttgatatctatgatgaggatgagaaccagagccccagaagcttccagaagaagaccagacactacttcatagctgcagtggagagactgtgggactatggcatgagcagcagcccccatgtactgagaaacagagcccagtcaggatctgtcccccagttcaagaaggtggtgttccaggagttcactgatgggagcttcacccagcccctgtacagaggtgaactgaatgaacacctgggcctgctgggcccctacatcagagcagaggtggaggacaacatcatggtgaccttcagaaaccaggccagcagaccctacagcttctacagcagcctgatcagctatgaagaggaccagagacagggagctgagcccagaaagaactttgtgaagcccaatgaaaccaagacctacttctggaaggtgcagcaccacatggcccccaccaaggatgaatttgactgcaaggcctgggcctacttctcagatgtagacctggagaaggatgtgcactcaggactgattggccccctgctggtgtgccacaccaacaccctgaacccagcacatggcagacaggtgacagttcaggagtttgcactgttcttcaccatctttgatgagaccaagagctggtacttcacagagaacatggagagaaactgcagagccccctgcaacatccagatggaggaccccaccttcaaggagaactacagattccatgctatcaatggctacatcatggacaccctgccagggctggtgatggcccaggaccagagaatcagatggtacctgctgagcatgggcagcaatgaaaacatccacagcatccacttcagtggccatgtgttcacagttagaaagaaggaggagtacaagatggccctgtacaacctgtaccctggtgtgtttgaaacagtggagatgctgcccagcaaggctggcatctggagagtggagtgcctgattggggaacacctgcatgcaggaatgagcaccctgttcctggtgtacagcaacaagtgccagacccccctgggcatggcctcaggccacatcagagacttccagatcactgccagtgggcagtatggtcagtgggcccccaagctggccagactgcactactctggcagcatcaatgcctggagcaccaaggagcccttcagctggatcaaggtggacctgctggcccccatgatcatccatggcatcaagacccagggggccagacagaagttcagcagcctgtacatcagccagttcatcatcatgtacagcctggatggcaagaagtggcagacctacagaggcaacagcactggaaccctgatggtgttctttggcaatgtagacagctcaggcatcaagcacaacatcttcaacccccccatcattgccagatacatcagactgcaccccacccactacagcatcagaagcaccctgagaatggagctgatgggctgtgacctgaacagctgcagcatgcccctgggcatggagagcaaggccatctctgatgcccagatcactgccagcagctacttcaccaacatgtttgccacctggagccccagcaaggccagactgcacctgcagggcagaagcaatgcttggagaccccaggtgaacaaccccaaggagtggctgcaggtggacttccagaagaccatgaaggtgactggtgtgaccacccagggtgtgaagagcctgctgaccagcatgtatgtcaaggagttcctgatcagcagcagccaggatggtcaccagtggaccctgttcttccagaatggcaaggtgaaggtgttccagggcaaccaggacagcttcaccccagtggtgaacagcctggacccccccctgctgaccagatacctgagaatccacccccagagctgggtgcaccagatagccctgagaatggaggtgctgggctgtgaggcccaggacctgtactga

SEQ ID NO: 14 - DNA, 4374 bp, synthetic construct
  source 1..4374: mol_type = other DNA
atgcagattgaactgagcacctgcttcttcctgtgcctgctgagattctgcttcagtgccaccagaagatactacctgggggcagtggagctgagctgggactacatgcagagtgacctgggggaactgcctgttgatgccagattcccccccagagtgcccaagagcttccccttcaacacctcagttgtgtacaagaagaccctgtttgtggagttcactgaccacctgttcaacattgctaagcccagacccccctggatgggcctgctgggccccaccatccaggctgaggtgtatgacactgtggtgatcaccctgaagaacatggccagccaccctgtgagcctgcatgcagtgggggtgagctactggaaggcctcagaaggggcagagtatgatgatcagaccagccagagagagaaggaggatgacaaggtgttccctggtggtagccacacctatgtgtggcaggtgctgaaggagaatggccccatggcctcagaccccctgtgcctgacctacagctacctgagccatgtggacctggtgaaggacctgaacagtggcctgattggagctctgctggtgtgcagagagggcagcctggccaaggagaagacccagaccctgcacaagttcatcctgctgtttgctgtgtttgatgagggcaagagctggcactcagagaccaagaacagcctgatgcaggacagagatgctgccagtgcaagagcctggcccaagatgcacactgtcaatggatatgtgaacagaagcctgccaggactgataggctgccacagaaagtctgtatactggcatgttataggtatgggcaccaccccagaggtgcacagcatcttcctggagggccacaccttcctggtgagaaaccacagacaggccagcctggagatcagccccatcaccttcctgactgcccagaccctgctgatggacctgggccagttcctgctgttctgccacatcagcagccaccagcatgatggtatggaggcctatgtgaaggtggacagctgccctgaggagccccagctgcagatgaagaacaatgaggaggcagaggactatgatgatgacctgacagacagtgagatggatgtggtgagatttgatgatgacaacagccccagcttcatccagatcagaagtgtggccaagaagcaccccaagacctgggtgcactacattgctgctgaagaggaggactgggactatgcacccctggtgctggccccagatgatagaagctacaagagccagtacctgaacaatggaccccagagaattggaagaaagtacaagaaggtgagattcatggcctacactgatgagaccttcaagaccagagaggccatccagcatgaatcagggatcctgggccccctgctgtatggagaggtgggagacaccctgctgatcatcttcaagaaccaggccagcagaccctacaacatctacccccatggtatcactgatgtaagacccctgtacagcagaagactgcccaagggagtgaagcacctgaaggacttccccatcctgcctggggaaatcttcaagtacaagtggacagtcacagttgaagatggacccaccaagagtgatcccagatgcctgaccagatactacagcagctttgtgaacatggagagagacctggcctcaggcctgataggccccctgctgatctgctacaaggagtctgttgaccagcagggcaaccagatcatgagtgacaagagaaatgtgatcctgttctcagtgtttgatgagaacagaagctggtacctgactgaaaacatccagagattcctgcccaacccagcaggggtgcagctggaggacccagagttccaggccagcaacatcatgcacagcatcaatggctatgtctttgatagcctgcagctgtctgtatgcctgcatgaggtggcctactggtacatcctgagcataggagcccagactgatttcctgagtgttttcttcagtggctacaccttcaagcacaagatggtgtatgaggacaccctgaccctgttccccttctctggtgagactgtgttcatgagcatggagaaccctggtctgtggatcctgggctgccacaactcagatttcagaaacagaggcatgacagccctgctgaaggtgagcagctgtgacaagaacactggggactactatgaagacagctatgaagacatctctgcttacctgctgagcaagaacaatgccattgagcccagaagcttcagccagaacccccctgtgctgaagagacaccagagagagatcaccagaaccaccctgcagtcagaccaggaggagatagactatgatgataccatctctgtggagatgaagaaggaggactttgatatctatgatgaggatgaaaaccagagccccagaagcttccagaagaagaccagacactacttcatagcagctgtagagagactgtgggactatggcatgagcagcagcccccatgtactgagaaacagagcccagagtggaagtgtgccccagttcaagaaggtggtgttccaggagttcacagatggcagcttcacccagcccctgtacagaggagaactgaatgagcacctgggcctgctgggcccctacatcagagctgaggtggaggacaacatcatggtgaccttcagaaaccaggccagcagaccctacagcttctacagcagcctgatcagctatgaggaggaccagagacagggagctgagcccagaaagaactttgtcaagcccaatgagaccaagacctacttctggaaggtgcagcaccacatggcccccaccaaggatgaatttgactgcaaggcctgggcctacttctcagatgtggacctggagaaggatgtgcactcaggtctgattggccccctgctggtgtgccacaccaacaccctgaacccagcccatggtagacaggtgactgtgcaggagtttgctctgttcttcaccatctttgatgagaccaagagctggtacttcacagagaacatggagagaaactgcagagccccctgcaacatccagatggaggaccccaccttcaaggagaactacagattccatgccatcaatgggtacatcatggacaccctgccaggtctggtgatggcccaggaccagagaatcagatggtacctgctgagcatgggcagcaatgaaaacatccacagcatccacttcagtggacatgtgttcacagttagaaagaaggaggagtacaagatggccctgtacaacctgtaccctggagtgtttgaaactgtggagatgctgcccagcaaggcaggcatctggagagtggagtgcctgataggggagcacctgcatgctggtatgagcaccctgttcctggtgtacagcaacaagtgccagacccccctgggcatggcctcaggccacatcagagacttccagatcactgctagtggccagtatggtcagtgggcccccaagctggccagactgcactactctggaagcatcaatgcctggagcaccaaggagcccttcagctggatcaaggtggacctgctggcccccatgatcatccatgggatcaagacccagggggccagacagaagttcagcagcctgtacatcagccagttcatcatcatgtacagcctggatggaaagaagtggcagacctacagaggcaacagcacaggcaccctgatggtgttctttggcaatgtagacagctcaggcatcaagcacaacatcttcaacccccccatcatagcaagatacatcagactgcaccccacccactacagcatcagaagcaccctgagaatggagctgatgggctgtgacctgaacagctgcagcatgcccctgggcatggagagcaaggccatctcagatgcccagatcacagccagcagctacttcaccaacatgtttgccacctggagccccagcaaggccagactgcacctgcagggcagaagcaatgcctggagaccccaggtgaacaaccccaaggagtggctgcaggtggacttccagaagaccatgaaggtgactggtgtgaccacccagggtgttaagagcctgctgaccagcatgtatgtgaaggagttcctgatcagcagcagccaggatgggcaccagtggaccctgttcttccagaatggaaaggtgaaggtgttccagggcaaccaggacagcttcacccctgttgtgaacagcctggacccccccctgctgaccagatacctgagaatccacccccagagctgggtgcaccagatagcactgagaatggaggtgctgggctgtgaggcccaggacctgtactga

SEQ ID NO: 15 - DNA, 4374 bp, synthetic construct
  source 1..4374: mol_type = other DNA
atgcagattgaactgagcacctgcttcttcctgtgcctgctgagattctgcttcagtgctaccagaagatactacctgggggcagtggagctgagctgggactacatgcagtctgacctgggagagctgccagtagatgccagattcccccccagagtgcccaagagcttccccttcaacacctctgtggtgtacaagaagaccctgtttgtggagttcacagaccacctgttcaacatagctaagcccagacccccctggatgggcctgctgggccccaccatccaggcagaggtgtatgacactgttgtgatcaccctgaagaacatggccagccacccagttagcctgcatgctgttggtgttagctactggaaggcctctgagggagcagagtatgatgaccagaccagccagagagagaaggaggatgacaaggtgttcccaggggggagccacacctatgtctggcaggtgctgaaggagaatggccccatggcctcagaccccctgtgcctgacctacagctacctgagccatgtggacctggtgaaggacctgaactctggcctgattggtgcactgctggtgtgcagagagggcagcctggccaaggagaagacccagaccctgcacaagttcatcctgctgtttgctgtctttgatgagggcaagagctggcactcagaaaccaagaacagcctgatgcaggacagagatgcagcttctgctagagcctggcccaagatgcacactgtgaatggctatgtcaacagaagcctgcctggcctgataggctgccacagaaagtctgtgtactggcatgtgattgggatgggcaccaccccagaggtgcacagcatcttcctggagggccacaccttcctggtgagaaaccacagacaggccagcctggagatcagccccatcaccttcctgactgcccagaccctgctgatggacctgggccagttcctgctgttctgccacatcagcagccaccagcatgatggtatggaggcctatgtgaaggtggacagctgcccagaggagccccagctgcagatgaagaacaatgaagaggcagaggactatgatgatgacctgacagactcagaaatggatgtggtgagatttgatgatgataacagccccagcttcatccagatcagatctgttgccaagaagcaccccaagacctgggtgcactacattgcagctgaggaggaggactgggactatgctcccctggtgctggccccagatgacagaagctacaagagccagtacctgaacaatggaccccagagaatagggagaaagtacaagaaggtgagattcatggcctacacagatgagaccttcaagaccagagaggccatccagcatgaatcagggatcctgggccccctgctgtatggggaggtgggtgacaccctgctgatcatcttcaagaaccaggccagcagaccctacaacatctacccccatggaatcacagatgtcagacccctgtacagcagaagactgcccaagggagtaaagcacctgaaggacttccccatcctgccaggtgagatcttcaagtacaagtggacagtaacagtggaggatggtcccaccaagtctgaccccagatgcctgaccagatactacagcagctttgttaacatggagagagacctggccagtggcctgataggccccctgctgatctgctacaaggagtctgtagaccagcagggcaaccagatcatgtcagataagagaaatgtaatcctgttcagtgtgtttgatgagaacagaagctggtacctgacagagaacatccagagattcctgcccaaccctgcaggtgtgcagctggaggaccctgaattccaggccagcaacatcatgcacagcatcaatggatatgtttttgacagcctgcagctgtcagtttgcctgcatgaggtggcctactggtacatcctgagcattggggcccagacagatttcctgtctgtgttcttcagtgggtacaccttcaagcacaagatggtgtatgaggacaccctgaccctgttccccttctcaggtgaaactgtgttcatgagcatggagaacccagggctgtggatcctgggctgccacaactcagacttcagaaacagaggcatgactgctctgctgaaggtgagcagctgtgacaagaacacaggtgactactatgaggacagctatgaagacatctcagcttacctgctgagcaagaacaatgccatagagcccagaagcttcagccagaaccccccagttctgaagagacaccagagagagatcaccagaaccaccctgcagagtgaccaggaggagattgactatgatgacaccatctctgtagagatgaagaaggaggactttgacatctatgatgaggatgagaaccagagccccagaagcttccagaagaagaccagacactacttcatagctgcagtggagagactgtgggactatggcatgagcagcagcccccatgtgctgagaaacagagcccagagtggctcagtgccccagttcaagaaggtggtgttccaggagttcacagatggcagcttcacccagcccctgtacagaggggagctgaatgagcacctgggcctgctgggcccctacatcagagctgaggtggaggacaacatcatggtgaccttcagaaaccaggccagcagaccctacagcttctacagcagcctgatcagctatgaggaggaccagagacagggggcagagcccagaaagaactttgtaaagcccaatgaaaccaagacctacttctggaaggtgcagcaccacatggcccccaccaaggatgaatttgactgcaaggcctgggcctacttcagtgatgtggacctggagaaggatgtgcactcaggcctgattggtcccctgctggtgtgccacaccaacaccctgaacccagcccatggcagacaggtgactgtacaggagtttgccctgttcttcaccatctttgatgagaccaagagctggtacttcacagagaacatggagagaaactgcagagccccctgcaacatccagatggaggaccccaccttcaaggagaactacagattccatgctatcaatgggtacatcatggacaccctgccaggcctggtgatggcccaggaccagagaatcagatggtacctgctgagcatgggcagcaatgagaacatccacagcatccacttcagtggccatgtgttcacagtgagaaagaaggaggagtacaagatggccctgtacaacctgtaccctggagtgtttgaaactgtggagatgctgcccagcaaggcaggtatctggagagtggagtgcctgattggtgagcacctgcatgctgggatgagcaccctgttcctggtgtacagcaacaagtgccagacccccctgggcatggcctcagggcacatcagagacttccagatcactgccagtgggcagtatgggcagtgggcccccaagctggccagactgcactacagtggaagcatcaatgcatggagcaccaaggagcccttcagctggatcaaggtggacctgctggcccccatgatcatccatgggatcaagacccagggggccagacagaagttcagcagcctgtacatcagccagttcatcatcatgtacagcctggatggcaagaagtggcagacctacagaggcaacagcacaggtaccctgatggtgttctttggaaatgtggacagctcaggtatcaagcacaacatcttcaacccccccatcatagcaagatacatcagactgcaccccacccactacagcatcagaagcaccctgagaatggagctgatgggctgtgacctgaacagctgcagcatgcccctgggcatggagagcaaggccatctcagatgctcagatcactgccagcagctacttcaccaacatgtttgccacctggagccccagcaaggccagactgcacctgcagggcagaagcaatgcctggagaccccaggtgaacaaccccaaggagtggctgcaggtggacttccagaagaccatgaaggtgacaggggttaccacccagggagtgaagagcctgctgaccagcatgtatgtaaaggagttcctgatcagcagcagccaggatggtcaccagtggaccctgttcttccagaatggaaaggtgaaggtgttccagggcaaccaggacagcttcaccccagtggtgaacagcctggacccccccctgctgaccagatacctgagaatccacccccagagctgggtgcaccagatagccctgagaatggaggtgctgggctgtgaagcccaggacctgtactga

SEQ ID NO: 16 - DNA, 4374 bp, synthetic construct
  source 1..4374: mol_type = other DNA
atgcaaatagaactctccacctgcttctttctgtgccttttgaggttctgcttttctgcaactagaagatactacctgggtgcagtagaactgtcatgggactatatgcaatcagatctaggagaactgccagtggatgccagatttcctccaagggtgcccaaatcatttccattcaacacaagtgttgtgtacaaaaagaccctgtttgtggaattcacagatcaccttttcaacatagccaagcctagaccaccttggatgggtctgttgggtcctaccatccaggcagaagtttatgatactgtggtcattacacttaagaacatggctagccatccagtcagtcttcatgctgttggggtatcctactggaaagcttcagagggagcagaatatgatgaccagacaagtcaaagggagaaagaagatgacaaagtcttccctggtggaagccatacctatgtctggcaggtcctgaaagagaatgggccaatggccagtgacccactgtgccttacctactcttatcttagtcatgtggacctggtaaaagacttgaatagtggcctcattggagcactactggtatgtagagaagggagcttggctaaggaaaagacacagactttgcacaaatttatactactttttgctgtatttgatgaggggaaaagttggcacagtgaaacaaagaactccttgatgcaggatagggatgcagcaagtgctagggcttggccaaaaatgcacacagttaatgggtatgttaacagatcactgcctgggctgattggttgccacagaaaatctgtctattggcatgtgattggaatgggcacaactcctgaagtgcactcaatattccttgaaggtcacacatttcttgtgagaaaccatagacaggcttccttggaaatcagccccataaccttccttacagctcaaacactcttgatggaccttggacagtttctactgttttgtcatatctcttcccaccaacatgatggaatggaagcctatgtcaaagtagacagctgtcctgaggaaccacaactacagatgaaaaataatgaagaagcagaagactatgatgatgatcttactgattcagaaatggatgtggtcaggtttgatgatgataactctccttcctttatccaaattagatctgtagcaaagaagcatcctaaaacttgggtacattacattgctgcagaagaggaggactgggactatgcacccttggtcttggcaccagatgatagaagttataaaagtcaatatttgaacaatggcccccagagaattggtaggaagtacaaaaaagtcagatttatggcatacacagatgaaacatttaagacaagagaagctattcagcatgagtcagggatcttgggacctttactttatggtgaagttggagatacactgttgattatatttaagaatcaagcaagcagaccatataacatctaccctcatggtatcacagatgtaagacccttgtattcaagaagattacccaaaggtgtaaaacatttgaaggattttccaattctgccaggggagatattcaaatataaatggacagtgacagtagaggatgggccaactaaatcagatcctaggtgcctgaccaggtattactctagttttgttaatatggagagagatctagctagtggactcattggacctctcctcatctgctacaaagaatcagttgatcaacagggaaaccagataatgtcagataagaggaatgtcatcctgttttctgtatttgatgaaaacaggagctggtacctcacagaaaatatacaaagatttctccccaatcctgctggagtgcagcttgaagatccagaattccaagcttcaaacatcatgcacagcatcaatggctatgtatttgattccttgcagttgtcagtgtgtttgcatgaggtggcatactggtacattctatctattggagctcagactgacttcctttctgtgttcttctcaggatatacattcaaacacaaaatggtctatgaagacacactcaccctattcccattctcaggggaaacagtcttcatgtcaatggaaaacccaggtctatggattttggggtgccacaactcagactttagaaacagaggcatgacagctttactgaaggttagtagttgtgataagaacactggggattattatgaggactcttatgaagatatttctgcatacttgctgagtaaaaacaatgcaattgaaccaagaagcttctctcaaaaccccccagtcttgaaaagacatcaaagagaaataaccagaactactcttcagtctgatcaagaggaaattgactatgatgataccatatcagttgaaatgaagaaggaagattttgacatttatgatgaagatgaaaatcagagccccaggagctttcaaaagaaaacaagacactattttattgcagcagtggagaggctctgggattatggtatgagtagctccccacatgtactaaggaacagggcccagagtggctctgtcccacagttcaagaaagtagttttccaggaatttacagatggctcctttactcagcctttatacaggggagaactaaatgaacatttgggactcctagggccatatataagagcagaggttgaagataatatcatggtaactttcaggaatcaggcctctaggccctattccttctattctagccttatttcttatgaggaagatcagaggcaaggagcagagcctaggaaaaactttgttaagcctaatgaaaccaaaacatacttttggaaagtgcaacatcatatggcccccaccaaagatgaatttgactgcaaagcctgggcatatttctctgatgtggacttagaaaaagatgttcactcaggcctgataggaccacttctggtctgccacactaacacactgaacccagcccatgggagacaagtgactgtacaggaatttgcactgtttttcaccatctttgatgaaacaaaaagctggtacttcactgaaaatatggaaagaaactgcagggctccctgcaatatccagatggaagatccaacatttaaagagaattatagattccatgctatcaatggctacataatggatacactaccaggcttagtaatggctcaggatcaaaggattagatggtatctgctcagcatgggcagtaatgagaacatccattctattcatttctcaggacatgtattcactgtaagaaaaaaagaggagtataaaatggcactgtacaatctctatccaggtgtttttgagacagtagaaatgttaccttcaaaagcaggaatttggagagtggaatgccttattggtgagcatctacatgctggaatgagcacactttttctggtgtacagcaataagtgtcagacccctctgggaatggcaagtggacacattagagactttcagattacagcttcaggacaatatggacagtgggccccaaagctggccagacttcattattcagggtcaatcaatgcatggagcaccaaggagccttttagctggatcaaggtggatctgttggcaccaatgattattcatggcatcaagactcagggagccagacagaagttctcaagcctctacatctctcagtttatcatcatgtattcccttgatgggaagaagtggcagacttatagaggaaatagtacaggaaccttaatggtcttctttgggaatgtggattcaagtgggataaaacacaatatttttaaccctccaattattgctaggtacatcaggttgcaccccactcattatagcattaggagcactcttagaatggagttgatgggctgtgatttaaatagttgcagtatgccattgggaatggagagtaaagcaatatcagatgcacagattactgcatcatcttactttactaatatgtttgcaacttggtctccatcaaaagcaagacttcacctccaagggaggtctaatgcttggagacctcaggtgaataatccaaaagagtggctgcaagtggacttccagaagacaatgaaagtcacaggtgtgaccactcagggtgtaaaatctctgcttaccagcatgtatgttaaggagttcctcatctccagcagtcaagatggacatcagtggactctcttttttcagaatggtaaagtaaaggtttttcagggaaatcaagactccttcacaccagtggttaacagtctagacccaccattactgactaggtaccttagaattcacccacagagttgggtgcaccagattgccctgaggatggaggttctgggctgtgaagcacaggacctctactga

SEQ ID NO: 17 - DNA, 4374 bp, synthetic construct
  source 1..4374: mol_type = other DNA
atgcaaatagagctctccacctgcttctttctgtgccttttgaggttctgcttttctgccacaaggagatactacctgggtgcagtggaactgtcatgggactatatgcaaagtgacctaggagagctgccagtggatgcaaggtttcctcctagagtgcccaaaagttttccattcaacacatcagttgtgtacaaaaagacactgtttgtagaattcacagatcaccttttcaacatagcaaagcccaggccaccatggatgggtctgctaggtcctaccatccaggcagaggtttatgatactgtggtcattacacttaagaacatggcttcacatcctgtcagtcttcatgctgtgggtgtatcctactggaaagcttcagagggagcagaatatgatgaccagactagtcaaagagagaaagaagatgataaagtcttcccaggtggatcccatacctatgtctggcaggtcctgaaagagaatggtcctatggccagtgatcctctgtgccttacttacagttatcttagtcatgttgacctggtaaaagacttgaattcaggactcattggagcactattggtatgtagagaagggtctttagctaaggaaaagacacagaccttgcacaaatttatactactttttgcagtatttgatgaagggaaaagttggcacagtgaaacaaagaactccttgatgcaggatagggatgcagcatctgctagggcctggcccaaaatgcacacagtcaatgggtatgtgaacaggtctctgcctggtctgattggatgccacagaaaatcagtatattggcatgtgataggaatgggcacaactcctgaggtgcactcaatattcttagaaggtcacacatttcttgtaagaaaccatagacaggcatccttggaaatctctcccataacattccttacagctcaaaccctcttgatggaccttggacagtttctactgttttgtcatatctcttcccaccaacatgatgggatggaagcatatgtcaaagtagacagctgtccagaggaaccacaactacagatgaaaaataatgaagaagcagaagactatgatgatgatcttactgattcagaaatggatgtagtcaggtttgatgatgataactctccttcctttatccaaattaggtctgttgccaagaagcatcctaaaacttgggtacattacattgctgcagaagaggaggactgggactatgcaccattagtactagcacctgatgacagaagttataaaagtcaatatttgaacaatggaccccagagaattgggaggaagtacaaaaaagtcagatttatggcatacactgatgagacatttaagactagagaagctattcagcatgaatcaggaatcttgggacctttactttatggggaagtaggagacacactgttgattatatttaagaatcaagcaagcagaccatataacatctacccacatggaatcacagatgttagacccttgtattcaaggaggttaccaaaaggtgtaaaacatttgaaggattttcctattctgccaggtgaaatattcaaatataaatggactgtgactgttgaagatggaccaactaaatcagaccccaggtgcctgaccaggtattactctagttttgttaatatggagagagatctagcttcaggactcataggccctctcctcatctgctacaaagaatcagtagatcaacagggaaaccagataatgtcagacaagaggaatgtaatcctgttttctgtatttgatgaaaacagaagctggtacctcacagagaatatacaaaggtttctccccaatcctgcaggagtgcagcttgaggatcctgagttccaagcttccaacatcatgcacagcatcaatgggtatgtgtttgattctttgcagttgtctgtttgtttgcatgaagtggcatactggtacattctatctattggtgctcagactgacttcctttctgtattcttctcaggctatactttcaaacacaaaatggtctatgaagacaccctcaccctattcccattctctggggaaactgtgttcatgtccatggaaaaccctggactatggattctgggctgccacaactctgactttagaaacagaggcatgactgctttactgaaggtttcaagttgtgacaagaacactggtgattattatgaagactcctatgaagatattagtgcctacttgctgagtaaaaacaatgcaatagaaccaaggagcttctctcaaaaccccccagtcttgaaaaggcatcaaagagaaataaccagaactactcttcagtcagatcaagaggaaattgactatgatgataccataagtgttgaaatgaagaaggaagattttgacatttatgatgaggatgaaaatcagagccccaggagctttcaaaagaaaacaagacactattttatagctgctgtagagagactctgggattatgggatgagttcatcccctcatgtactaaggaacagggcccagtcaggttcagtcccccagttcaagaaagttgtcttccaggaatttacagatgggtcctttactcagcctttatacaggggagaactaaatgaacatttgggactcctgggaccatatataagagcagaagttgaagataatatcatggtaactttcaggaatcaggcctctagaccctattccttctattctagccttatttcttatgaagaagatcagaggcaaggagcagaacctagaaaaaactttgtcaagcctaatgaaactaaaacttacttttggaaagtgcaacatcatatggctcccactaaagatgagtttgattgcaaagcctgggcatatttctcagatgttgacctagagaaagatgtacactcaggactgattggacctcttctggtctgccacactaacacactgaaccctgctcatggtagacaagtgactgtacaggaatttgccctgtttttcaccatctttgatgagacaaaaagctggtacttcacagaaaatatggaaagaaactgcagggctccctgcaatatccagatggaagatcctacctttaaagagaattataggttccatgcaatcaatggctacataatggatacactacctgggttagtaatggctcaggatcaaaggattaggtggtatctgctcagcatgggctccaatgagaacatccattccattcatttctcaggacatgtgttcacagttaggaaaaaagaggagtataaaatggcactgtacaatctctatccaggtgtttttgaaacagtagaaatgttacccagtaaagctggaatttggagggtggagtgccttataggtgagcatctacatgcaggaatgagcacactttttctggtgtacagcaataagtgtcagacacccctgggaatggcctcaggacacattagggactttcagattacagccagtggacaatatggtcagtgggccccaaagctggccagacttcattatagtggttcaatcaatgcttggagcactaaggagcccttttcttggatcaaggtggatctgctggctcccatgattattcatggcatcaagacacagggtgctaggcagaagttcagcagtctctacatctctcagtttatcatcatgtattcacttgatgggaagaagtggcagacttatagaggaaattccacaggaaccttaatggtcttctttggaaatgtggattcatctggaataaaacacaatatttttaaccctccaattattgctagatacatcaggttgcacccaactcattatagcattagaagcactcttagaatggagttgatgggctgtgatttaaatagttgctccatgcccttgggaatggagagtaaagcaatatcagatgcccagattacagcttcatcatactttaccaatatgtttgcaacatggtctccatcaaaagcaagacttcacctccaagggagaagtaatgcctggagacctcaggtgaataatccaaaagagtggctgcaagtggacttccagaagactatgaaagtcacaggagtaacaactcagggagttaaatctctgcttaccagcatgtatgtaaaggagttcctcatctccagcagtcaagatgggcatcagtggactctcttttttcagaatggaaaagtaaaggtttttcagggaaatcaagactccttcacaccagtggttaacagtctggaccctcccttactgactagataccttaggattcaccctcagagttgggtgcaccagattgcactgagaatggaggttctgggctgtgaggcacaggacctctactga

SEQ ID NO: 18 - DNA, 4374 bp, synthetic construct
  source 1..4374: mol_type = other DNA
atgcaaatagagctctccacctgcttctttctgtgccttttgaggttctgctttagtgcaacaagaagatactacctgggtgcagtagaactgtcatgggactatatgcaatctgatttaggggagctgcctgtggatgcaagatttccacccagggtgcctaaaagctttcccttcaacacttcagttgtgtacaaaaagaccctgtttgtagaattcactgatcaccttttcaacattgctaagccaaggccaccctggatgggtctgcttggtcctaccatccaggcagaagtttatgatactgtggtcattacacttaagaacatggcttcccatccagtgagtcttcatgctgtgggtgtatcctactggaaagcttctgagggagctgaatatgatgaccagaccagtcaaagggagaaagaagatgacaaagtcttcccaggtggaagccatacctatgtgtggcaggtcctgaaagagaatggtccaatggcctctgatcctctgtgccttacctactcctatctttcacatgttgacctggtaaaagacttgaattcaggcctcattggggccctactagtttgtagagaagggtctctggctaaggaaaagactcagacattgcacaaatttatactactttttgctgtatttgatgaggggaaaagttggcactcagagacaaagaactccttgatgcaggatagagatgctgcttcagctagagcatggcctaaaatgcacactgtcaatggatatgtaaacagaagcctgccaggtctgattggttgccacaggaaatctgtctattggcatgtgattggcatgggcaccactccagaagtgcactcaatattcttagagggtcacacatttcttgtaagaaaccatagacaggcttccttggaaatctcacctataaccttccttacagctcaaaccctcttgatggacctgggacagtttctactgttttgtcatatctcttcccaccaacatgatggaatggaagcttatgtcaaagtagacagctgtccagaggaaccccaactacagatgaaaaataatgaagaagctgaagactatgatgatgatcttactgatagtgaaatggatgtagtcaggtttgatgatgacaactctccttcctttatccaaattaggtcagttgctaagaagcatcctaaaacttgggtacattacattgcagcagaagaggaggactgggactatgccccattagtcctagcccctgatgatagaagttataaaagtcaatatttgaacaatggaccacagagaattggtaggaagtacaaaaaagtcagatttatggcatacactgatgagacctttaagacaagggaagctattcagcatgaatctggaatcttgggacctttactttatggagaagttggggacacactgttgattatatttaagaatcaagcaagcagaccatataacatctacccccatggaatcactgatgtcaggcccttgtattcaagaaggttacctaaaggtgtaaaacatttgaaggattttcctattctgccaggtgaaatattcaaatataaatggacagtgactgtagaagatggcccaactaaaagtgatcccaggtgcctgaccagatattactctagttttgtcaatatggagagagatctagcttcaggactcattggtcctctcctcatctgctacaaagaatcagtagatcaacagggaaaccagataatgagtgataagaggaatgtcatcctgtttagtgtatttgatgaaaacagaagctggtacctcacagaaaatatacaaagatttctccccaatccagctggggtgcagttggaggatccagaattccaagcaagtaacatcatgcacagcatcaatgggtatgtatttgactctttgcagttgtcagtatgtttgcatgaagtggcatactggtacattctatcaattggggcccagactgatttccttagtgtcttcttctctggttatacattcaaacacaaaatggtctatgaagacaccctcaccctattcccattctcaggggaaactgtcttcatgagtatggaaaacccaggtctatggattttggggtgccacaactcagactttaggaacagaggcatgactgcattactgaaggtttcaagttgtgataagaacactggtgattattatgaagacagttatgaggatatttcagcatacttgctgagtaaaaacaatgcaatagaaccaagaagcttctctcaaaacccaccagtcttgaaaagacatcaaagagaaataaccaggactactcttcagtctgatcaagaggaaattgattatgatgacaccatatcagttgaaatgaagaaggaagattttgatatttatgatgaggatgaaaatcagagcccaaggagctttcaaaagaaaaccagacactattttattgcagcagtagagagactctgggattatgggatgagttcatccccccatgttctaagaaacagggcacagagtggcagtgtcccacagttcaagaaagtagttttccaggaatttacagatggttcctttactcagccattatacaggggagaactaaatgaacatttgggactcttagggccatatataagagctgaagttgaagataatatcatggtaactttcaggaatcaggcctctagaccatattccttctatagtagccttatttcttatgaggaagatcagaggcaaggagcagagcctaggaaaaactttgttaagcctaatgaaacaaaaacttacttttggaaagtgcaacatcatatggcacccaccaaagatgagtttgactgcaaagcctgggcctatttctctgatgttgacttagaaaaagatgtgcactctgggctgattggaccacttctggtctgccacactaacacactgaacccagcacatggcagacaagtgactgtccaggaatttgctctgtttttcaccatctttgatgagacaaaaagctggtacttcactgaaaatatggaaaggaactgcagggctccctgcaatatccagatggaagatccaacctttaaagagaattatagattccatgccatcaatgggtacataatggatacactacctggcttagtaatggctcaggatcaaaggattaggtggtatctgctcagcatgggctccaatgaaaacatccatagcattcatttcagtggacatgttttcactgtaaggaaaaaagaggagtataaaatggcactgtacaatctctatccaggagtttttgagacagttgaaatgttaccatctaaagcaggaatttggagggttgaatgccttattggggaacatctacatgcaggtatgagcacactttttctggtgtacagcaataagtgtcagactccactgggaatggcatctggccacattagagattttcagattactgcatcagggcaatatggacagtgggccccaaagctggccagacttcattatagtggatcaatcaatgcttggagcaccaaggagcccttttcttggatcaaggtggatctgcttgcacccatgattattcatgggatcaagacccagggtgccagacagaagttcagcagcctctacatctctcagtttatcatcatgtatagccttgatggaaagaagtggcagacttataggggaaatagcacaggaaccttaatggtcttctttggtaatgttgattcaagtgggataaaacacaatatttttaaccctccaattatagctagatacatcagattgcaccccactcattatagcattaggagcactcttaggatggagttgatgggctgtgatttaaatagttgcagtatgcccttgggaatggagagtaaagcaatatcagatgctcagattactgcatcaagctactttactaatatgtttgctacatggtctccatcaaaagctagacttcacctccaagggagatccaatgcttggagacctcaggtgaataatccaaaagagtggctgcaagtggacttccagaagactatgaaagtcacaggggtaacaacccagggagtaaaatctctgcttaccagcatgtatgtcaaggagttcctcatctccagctcacaagatgggcatcagtggactctcttttttcagaatgggaaagtaaaggtttttcagggaaatcaagactccttcacaccagtggtgaactctttggacccacctttactgactagataccttaggattcacccacagagttgggtgcaccagatagctctgagaatggaggttctgggctgtgaggcccaggacctctactga

SEQ ID NO: 19 - protein, 1457 aa, Homo sapiens
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLQMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQQGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 20 - protein, 19 aa, Homo sapiens
MQIELSTCFFLCLLRFCFS

SEQ ID NO: 21 - DNA, 8218 bp, synthetic construct
  source 1..8218: mol_type = other DNA
tctagagctagcatatggatccatcgatttagggataacagggtaattatcagcacacaattgcccattatacgcgcgtataatggactattgtgtgctgatatctgtacacttaagggctagatcttagcttacgtcactagagggtccacgtttagtttttaagatccattgatctcctaaacgctgcaagattcgcaacctggtatacttagcctaggcgctaggtcctagtgcagcgggactttttttctaaagtcgttgagaggaggagtcgtcagaccagatagctttgatgtcctgatcggaaggatcgttggcccccctgcaggcagctgttaattaactgcgcgctcgctcgctcactgaggccgcccgggcaaagcccgggcgtcgggcgacctttggtcgcccggcctcagtgagcgagcgagcgcgcagagagggagtggccaactccatcactaggggttcctgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgccaccatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgcagatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagcagggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaaccccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactgaaataaaagatctttattttcattagatctgtgtgttggttttttgtgtgaggaacccctagtgatggagttggccactccctctctgcgcgctcgctcgctcactgaggccgggcgaccaaaggtcgcccgacgcccgggctttgcccgggcggcctcagtgagcgagcgagcgcgcagttaattaaggcgccctaggccgacccttagactctgtactcagttctataaacgagccattggatacgagatccgtagattgataagggacacggaatatccccggacgcaatagacaccggtggacagcttggtatcctgagcacagtcgcgcgtccgaatctagctctactttagaggccccggattctgatggtcgtagaccgcagaaccgattggggggatgagatctactagttatcagcacacaattgcccattatacgcgcgtataatggactattgtgtgctgatatagggataacagggtaattctagagctagcatatggatccatcgatttgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagcgtataatggtctagagctagcatatggatccatcgattccattatacgcctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 22 - skipped

SEQ ID NO: 23 - protein, 14 aa, synthetic construct
SFSQNPPVLKRHQR

SEQ ID NO: 24 - protein, 2351 aa, Homo sapiens
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 25 - protein, 214 aa, Homo sapiens
MRIQDPGKVFFGNVDSSGIKHNIFNPPIIARYIRLHPTHYSIRSTLRMELMGCDLNSCSMLGMESKAISDAQITASSYFTNMFATWSPSKARLHLQGRSNAWRPQVNNPKEWLQVDFQKMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 26 - DNA, 6314 bp, synthetic construct
  source 1..6314: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacggcgcgccacgcgttctaggagttaatttttaaaaagcagtcaaaagtccaagtgcccttgcgagcatttactctctctgtttgctctggttaataatctcaggagcacaaacattccttactagttctaggagttaatttttaaaaagcagtcaaaagtccaagtgcccttgcgagcatttactctctctgtttgctctggttaataatctcaggagcacaaacattccttactagttctagagcggccgccagtgtgctggaattcggcttttttagggctggaagctacctttgacatcatttcctctgcgaatgcatgtataatttctacagaacctattagaaaggatcacccagcctctgcttttgtacaactttcccttaaaaaactgccaatcccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactgagagtgctctagttctgcaatacaggacatgctataaaaatggaaagatgttgctttctgagagatcaggtggattcttgggcatttgcttaagcttggtaccgccaccatgcagattgagctgagcacctgcttcttcctgtgcctgctgagattctgcttctctgccaccagaagatactacctgggtgcagtggagctgagctgggactacatgcagagtgacctgggtgagctgcctgttgatgccagattcccccccagagtgcccaagagcttccccttcaacacctctgttgtgtacaagaagaccctgtttgttgagttcactgaccacctgttcaacattgccaagcccagacccccctggatgggcctgctgggccccaccatccaggcagaggtgtatgatactgtggtgatcaccctgaagaacatggccagccacccagttagcctgcatgcagtgggggtcagctactggaaggcctcagaaggggctgagtatgatgatcagaccagccagagagagaaggaggatgacaaggtgttcccagggggtagccacacctatgtatggcaggtgctgaaggagaatggccccatggcctctgaccccctgtgcctgacctacagctacctgagccatgtggacctggtgaaggacctgaacagtggactgattggtgccctgctggtgtgcagagagggcagcctggccaaggagaagacccagaccctgcacaagttcatcctgctgtttgctgtgtttgatgaaggcaagagctggcacagtgagaccaagaacagcctgatgcaggacagagatgctgcatcagctagagcctggcccaagatgcacactgtcaatggttatgtaaacagaagcctgcctggcctgattggctgccacagaaagtcagtatactggcatgttataggcatgggcaccaccccagaggtgcacagcatcttcctggagggccacaccttcctggtgagaaaccacagacaggccagcctggagatcagccccatcaccttcctgacagcccagaccctgctgatggacctgggccagttcctgctgttctgccacatcagcagccaccagcatgatgggatggaggcctatgtaaaggtggacagctgcccagaggagccccagctgcagatgaagaacaatgaagaggctgaagactatgatgatgatctgactgattcagagatggatgttgtgagatttgatgatgacaacagccccagcttcatccagatcagatcagtggccaagaagcaccccaagacctgggtgcactacatagctgcagaggaggaggactgggactatgcccccctggtgctggccccagatgatagaagctacaagagccagtacctgaacaatggcccccagagaatagggagaaagtacaagaaggtgagattcatggcctacacagatgagaccttcaagaccagagaggccatccagcatgagtcaggtatcctgggccccctgctgtatggggaggtgggtgacaccctgctgatcatcttcaagaaccaggccagcagaccctacaacatctacccccatgggatcacagatgtcagacccctgtacagcagaagactgcccaagggagtcaagcacctgaaggacttccccatcctgcctggagaaatcttcaagtacaagtggacagtgactgtggaggatggtcccaccaagagtgaccccagatgcctgaccagatactacagcagctttgtcaacatggagagagacctggccagtggcctgataggccccctgctgatctgctacaaggagtctgtggaccagcagggcaaccagatcatgtctgacaagagaaatgttatcctgttcagtgtatttgatgagaacagaagctggtacctgactgagaacatccagagattcctgcccaacccagctggggtccagctggaggaccctgagttccaggccagcaacatcatgcacagcatcaatggctatgtgtttgacagcctgcagctgtctgtctgcctgcatgaagtggcctactggtacatcctgagcataggagcccagacagacttcctgagtgtattcttctctggctacaccttcaagcacaagatggtgtatgaggacaccctgaccctgttccccttctctggagagactgtattcatgagcatggagaaccctggcctgtggatcctgggctgccacaactctgacttcagaaacagaggcatgactgccctgctgaaggtgagcagctgtgataagaacacaggggactactatgaggacagctatgaggacatctcagcctacctgctgagcaagaacaatgccattgagcccagaagcttcagccagaacccccctgtgctgaagagacaccagagagagatcaccagaaccaccctgcagagtgaccaggaggagattgactatgatgacaccatctctgttgagatgaagaaggaggactttgacatctatgatgaggatgagaaccagagccccagaagcttccagaagaagaccagacactacttcattgcagctgtggagagactgtgggactatggcatgagcagcagcccccatgtgctgagaaacagagcccagtcaggctctgtgccccagttcaagaaggtggtgttccaggagttcacagatgggagcttcacccagcccctgtacagaggtgaactgaatgagcacctgggcctgctgggcccctacatcagagctgaagtggaggacaacatcatggtgaccttcagaaaccaggccagcagaccctacagcttctacagcagcctgatcagctatgaagaggaccagagacagggtgctgagcccagaaagaactttgtgaagcccaatgaaaccaagacctacttctggaaggtgcagcaccacatggcccccaccaaggatgaatttgactgcaaggcctgggcctacttctcagatgtagacctggagaaggatgttcactcaggcctgattgggcccctgctggtgtgccacaccaacaccctgaacccagcccatggtagacaggtgacagtgcaggagtttgccctgttcttcaccatctttgatgagaccaagagctggtacttcactgagaacatggagagaaactgcagagccccctgcaacatccagatggaggaccccaccttcaaggagaactacagattccatgccatcaatgggtacatcatggacaccctgccaggcctggtgatggcccaggaccagagaatcagatggtacctgctgagcatgggcagcaatgagaacatccacagcatccacttcagtggtcatgtcttcacagtgagaaagaaggaggagtacaagatggccctgtacaacctgtacccaggggtgtttgaaactgtagagatgctgcccagcaaggcaggcatctggagagtggagtgcctgataggagaacacctgcatgcaggaatgagcaccctgttcctggtgtacagcaacaagtgccagacccccctgggcatggcctctgggcacatcagagacttccagatcactgcctctggccagtatgggcagtgggcccccaagctggccagactgcactactcaggtagcatcaatgcctggagcaccaaggagcccttcagctggatcaaggtggacctgctggcccccatgatcatccatggtatcaagacccagggtgccagacagaagttcagcagcctgtacatcagccagttcatcatcatgtacagcctggatggcaagaagtggcagacctacagaggcaacagcactggcaccctgatggtgttctttggcaatgtggacagctctggcatcaagcacaacatcttcaacccccccatcatagccagatacatcagactgcaccccacccactacagcatcagaagcaccctgagaatggagctgatgggctgtgatctgaacagctgcagcatgcccctgggcatggagagcaaggccatctctgatgctcagatcactgcaagcagctacttcaccaacatgtttgccacctggagccccagcaaggccagactgcacctgcagggcagaagcaatgcctggagaccccaggtgaacaaccccaaggagtggctgcaggtggacttccagaagaccatgaaggtgacaggtgtcaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgagaatccacccccagagctgggtgcaccagattgccctgagaatggaggtgctgggctgtgaggcccaggacctgtactgatttaaatctcgagccgggcggagtgtgttagtctctccagagggaggctggttccccagggaagcagagcctgtgtgcgggcagcagctgtgtgcgggcctgggggttgttaagtgcaattatttttaataaaaggggcatttggaaaaaaaaaaaaaaggtagcagtcgacagatgaattctgcagatctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaaaaaagagaaatgttctggcacctgcacttgcactggggacagcctattttgctagtttgttttgtttcgttttgttttgatggagagcgtatgttgtttaaacgcggccgcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 27 - DNA, 6314 bp, synthetic construct
  source 1..6314: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacggcgcgccacgcgttctaggagttaatttttaaaaagcagtcaaaagtccaagtgcccttgcgagcatttactctctctgtttgctctggttaataatctcaggagcacaaacattccttactagttctaggagttaatttttaaaaagcagtcaaaagtccaagtgcccttgcgagcatttactctctctgtttgctctggttaataatctcaggagcacaaacattccttactagttctagagcggccgccagtgtgctggaattcggcttttttagggctggaagctacctttgacatcatttcctctgcgaatgcatgtataatttctacagaacctattagaaaggatcacccagcctctgcttttgtacaactttcccttaaaaaactgccaatcccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactgagagtgctctagttctgcaatacaggacatgctataaaaatggaaagatgttgctttctgagagatcaggtggattcttgggcatttgcttaagcttggtaccgccaccatgcagattgagctgagcacctgcttcttcctgtgcctgctgagattctgcttcagtgccaccagaagatactacctgggagcagtggagctgagctgggactacatgcagagtgatctgggagagctgccagtagatgccagattcccccccagagtgcccaagagcttccccttcaacacctcagtggtgtacaagaagaccctgtttgttgagttcactgaccacctgttcaacattgcaaagcccagacccccctggatgggcctgctgggccccaccatccaggcagaagtgtatgacacagtagtgatcaccctgaagaacatggccagccaccctgtcagcctgcatgcagttggtgtaagctactggaaggcctctgagggtgctgaatatgatgaccagaccagccagagagagaaggaggatgataaggtgttcccagggggcagccacacctatgtttggcaggtgctgaaggagaatgggcccatggcctctgatcccctgtgcctgacctacagctacctgagccatgtggacctggtgaaggacctgaactctgggctgataggagccctgctggtgtgcagagagggcagcctggccaaggagaagacccagaccctgcacaagttcatcctgctgtttgctgtgtttgatgaaggcaagagctggcacagtgaaaccaagaacagcctgatgcaggacagagatgcagctagtgccagagcctggcccaagatgcacacagtaaatggttatgtgaacagaagcctgcctggcctgataggctgccacagaaagtctgtgtactggcatgttataggtatgggcaccacccctgaggtgcacagcatcttcctggagggccacaccttcctggtgagaaaccacagacaggccagcctggagatcagccccatcaccttcctgactgcccagaccctgctgatggacctgggccagttcctgctgttctgccacatcagcagccaccagcatgatggaatggaggcctatgtcaaggtggacagctgcccagaggagccccagctgcagatgaagaacaatgaggaggcagaggactatgatgatgacctgactgacagtgaaatggatgtggtcagatttgatgatgataacagccccagcttcatccagatcagatcagtggccaagaagcaccccaagacctgggtgcactacattgcagctgaggaggaggactgggactatgcacccctggtgctggccccagatgatagaagctacaagagccagtacctgaacaatggaccccagagaattggcagaaagtacaagaaggtgagattcatggcctacactgatgagaccttcaagaccagagaggccatccagcatgaatcaggcatcctgggccccctgctgtatggggaggtgggagacaccctgctgatcatcttcaagaaccaggccagcagaccctacaacatctacccccatggaatcacagatgttagacccctgtacagcagaagactgcccaagggggtgaagcacctgaaggacttccccatcctgcctggggagatcttcaagtacaagtggacagttacagtagaggatggccccaccaagagtgaccccagatgcctgaccagatactacagcagctttgtaaacatggagagagacctggcctctggcctgataggccccctgctgatctgctacaaggagtcagttgaccagcagggcaaccagatcatgagtgataagagaaatgtcatcctgttcagtgtgtttgatgagaacagaagctggtacctgacagagaacatccagagattcctgcccaaccctgctggagtgcagctggaggacccagaattccaggccagcaacatcatgcacagcatcaatggttatgtctttgatagcctgcagctgtcagtttgcctgcatgaggtggcctactggtacatcctgagcattggggcccagactgacttcctgagtgtattcttcagtggctacaccttcaagcacaagatggtgtatgaggacaccctgaccctgttccccttctctggagagactgtgttcatgagcatggagaaccctgggctgtggatcctgggctgccacaactcagacttcagaaacagaggcatgacagcactgctgaaggtgagcagctgtgataagaacactggtgactactatgaggacagctatgaagacatctcagcctacctgctgagcaagaacaatgccatagagcccagaagcttcagccagaaccccccagttctgaagagacaccagagagagatcaccagaaccaccctgcagagtgaccaggaggagatagattatgatgataccatctcagttgagatgaagaaggaggactttgatatctatgatgaggatgagaaccagagccccagaagcttccagaagaagaccagacactacttcatagcagctgtggagagactgtgggactatggcatgagcagcagcccccatgttctgagaaacagagcccagagtggatcagtaccccagttcaagaaggtggtgttccaggagttcactgatgggagcttcacccagcccctgtacagaggtgaactgaatgagcacctgggcctgctgggcccctacatcagagctgaagtggaggacaacatcatggtgaccttcagaaaccaggccagcagaccctacagcttctacagcagcctgatcagctatgaggaggaccagagacagggagctgagcccagaaagaactttgtcaagcccaatgaaaccaagacctacttctggaaggtgcagcaccacatggcccccaccaaggatgagtttgactgcaaggcctgggcctacttcagtgatgtagacctggagaaggatgtccactcaggcctgattggccccctgctggtgtgccacaccaacaccctgaacccagcacatgggagacaggtgactgtacaggagtttgctctgttcttcaccatctttgatgagaccaagagctggtacttcacagaaaacatggagagaaactgcagagccccctgcaacatccagatggaggaccccaccttcaaggagaactacagattccatgccatcaatggttacatcatggacaccctgcctggcctggtgatggcccaggaccagagaatcagatggtacctgctgagcatgggcagcaatgaaaacatccacagcatccacttctcagggcatgtgttcactgtgagaaagaaggaggagtacaagatggccctgtacaacctgtacccaggtgtgtttgaaactgtggagatgctgcccagcaaggcaggcatctggagagtggagtgcctgataggggagcacctgcatgcagggatgagcaccctgttcctggtgtacagcaacaagtgccagacccccctgggcatggcctcaggccacatcagagacttccagatcacagcttctggacagtatggccagtgggcccccaagctggccagactgcactacagtggcagcatcaatgcctggagcaccaaggagcccttcagctggatcaaggtggacctgctggcccccatgatcatccatggcatcaagacccagggagccagacagaagttcagcagcctgtacatcagccagttcatcatcatgtacagcctggatggcaagaagtggcagacctacagaggcaacagcacaggcaccctgatggtgttctttgggaatgtggacagcagtggaatcaagcacaacatcttcaacccccccatcattgcaagatacatcagactgcaccccacccactacagcatcagaagcaccctgagaatggagctgatgggctgtgacctgaacagctgcagcatgcccctgggcatggagagcaaggccatctctgatgcccagatcacagctagcagctacttcaccaacatgtttgccacctggagccccagcaaggccagactgcacctgcagggcagaagcaatgcctggagaccccaggtgaacaaccccaaggagtggctgcaggtggacttccagaagaccatgaaggtgactggtgttaccacccagggggtcaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgagaatccacccccagagctgggtgcaccagatagcactgagaatggaggtgctgggctgtgaggcccaggacctgtactgatttaaatctcgagccgggcggagtgtgttagtctctccagagggaggctggttccccagggaagcagagcctgtgtgcgggcagcagctgtgtgcgggcctgggggttgttaagtgcaattatttttaataaaaggggcatttggaaaaaaaaaaaaaaggtagcagtcgacagatgaattctgcagatctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaaaaaagagaaatgttctggcacctgcacttgcactggggacagcctattttgctagtttgttttgtttcgttttgttttgatggagagcgtatgttgtttaaacgcggccgcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 28 - DNA, 6314 bp, synthetic construct
  source 1..6314: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacggcgcgccacgcgttctaggagttaatttttaaaaagcagtcaaaagtccaagtgcccttgcgagcatttactctctctgtttgctctggttaataatctcaggagcacaaacattccttactagttctaggagttaatttttaaaaagcagtcaaaagtccaagtgcccttgcgagcatttactctctctgtttgctctggttaataatctcaggagcacaaacattccttactagttctagagcggccgccagtgtgctggaattcggcttttttagggctggaagctacctttgacatcatttcctctgcgaatgcatgtataatttctacagaacctattagaaaggatcacccagcctctgcttttgtacaactttcccttaaaaaactgccaatcccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactgagagtgctctagttctgcaatacaggacatgctataaaaatggaaagatgttgctttctgagagatcaggtggattcttgggcatttgcttaagcttggtaccgccaccatgcaaattgagttgtccacctgcttcttcttgtgcttgttgaggttctgcttcagtgccaccaggaggtactacttgggggctgtggagttgtcctgggactacatgcagtcagacttgggggagttgcctgtggatgccaggttcccccccagggtccccaagtccttccccttcaacaccagtgtggtctacaagaagaccttgtttgtggagttcactgaccacttgttcaacattgccaagcccaggcccccctggatgggcttgttgggccccaccatccaggctgaggtctatgacacagtggtcatcaccttgaagaacatggcctcccaccctgtctccttgcatgcagtgggggtctcctactggaaggccagtgagggggctgagtatgatgaccagacctcccagagggagaaggaggatgacaaggtcttccctgggggctcccacacctatgtctggcaggtcttgaaggagaatggccccatggcctctgaccccttgtgcttgacctactcctacttgtcccatgtggacttggtcaaggacttgaactcaggcttgattggggccttgttggtctgcagggagggctccttggccaaggagaagacccagaccttgcacaagttcatcttgttgtttgcagtctttgatgagggcaagtcctggcacagtgagaccaagaactccttgatgcaggacagggatgctgccagtgccagggcctggcccaagatgcacactgtcaatggctatgtcaacaggtccttgcctggcttgattggctgccacaggaagtcagtctactggcatgtcattggcatgggcaccacccctgaggtccactccatcttcttggagggccacaccttcttggtcaggaaccacaggcaggcctccttggagatctcccccatcaccttcttgacagcccagaccttgttgatggacttgggccagttcttgttgttctgccacatctcctcccaccagcatgatggcatggaggcctatgtcaaggtggactcctgccctgaggagccccagttgcagatgaagaacaatgaggaggcagaggactatgatgatgacttgactgactctgagatggatgtggtcaggtttgatgatgacaactccccctccttcatccagatcaggagtgtggccaagaagcaccccaagacctgggtccactacatagcagcagaggaggaggactgggactatgcccccttggtcttggcccctgatgacaggtcctacaagtcccagtacttgaacaatggcccccagaggattggcaggaagtacaagaaggtcaggttcatggcctacacagatgagaccttcaagaccagggaggccatccagcatgagtcaggcatcttgggccccttgttgtatggggaggtgggggacaccttgttgatcatcttcaagaaccaggcctccaggccctacaacatctacccccatggcatcactgatgtcaggcccttgtactccaggaggttgcccaagggggtcaagcacttgaaggacttccccatcttgccaggggagatcttcaagtacaagtggacagtcactgtggaggatggccccaccaagtcagaccccaggtgcttgaccaggtactactcctcctttgtcaacatggagagggacttggccagtggcttgattggccccttgttgatctgctacaaggagtctgtggaccagcagggcaaccagatcatgtcagacaagaggaatgtcatcttgttcagtgtctttgatgagaacaggtcctggtacttgacagagaacatccagaggttcttgcccaacccagctggggtccagttggaggaccctgagttccaggcctccaacatcatgcactccatcaatggctatgtctttgactccttgcagttgtcagtctgcttgcatgaggtggcctactggtacatcttgtccattggggcccagactgacttcttgtctgtcttcttcagtggctacaccttcaagcacaagatggtctatgaggacaccttgaccttgttccccttcagtggggagactgtcttcatgtccatggagaacccaggcttgtggatcttgggctgccacaactcagacttcaggaacaggggcatgacagccttgttgaaggtctcctcctgtgacaagaacacaggggactactatgaggactcctatgaggacatctcagcctacttgttgtccaagaacaatgccatagagcccaggtccttctcccagaacccccctgtcttgaagaggcaccagagggagatcaccaggaccaccttgcagtcagaccaggaggagatagactatgatgacaccatctcagtggagatgaagaaggaggactttgacatctatgatgaggatgagaaccagtcccccaggtccttccagaagaagaccaggcactacttcattgcagcagtggagaggttgtgggactatggcatgtcctcctccccccatgtcttgaggaacagggcccagtcaggcagtgtcccccagttcaagaaggtggtcttccaggagttcactgatggctccttcacccagcccttgtacaggggggagttgaatgagcacttgggcttgttgggcccctacatcagggctgaggtggaggacaacatcatggtcaccttcaggaaccaggcctccaggccctactccttctactcctccttgatctcctatgaggaggaccagaggcagggggcagagcccaggaagaactttgtcaagcccaatgagaccaagacctacttctggaaggtccagcaccacatggcccccaccaaggatgagtttgactgcaaggcctgggcctacttctctgatgtggacttggagaaggatgtccactctggcttgattggccccttgttggtctgccacaccaacaccttgaaccctgcccatggcaggcaggtcactgtccaggagtttgccttgttcttcaccatctttgatgagaccaagtcctggtacttcacagagaacatggagaggaactgcagggccccctgcaacatccagatggaggaccccaccttcaaggagaactacaggttccatgccatcaatggctacatcatggacaccttgccaggcttggtcatggcccaggaccagaggatcaggtggtacttgttgtccatgggctccaatgagaacatccactccatccacttcagtggccatgtcttcactgtcaggaagaaggaggagtacaagatggccttgtacaacttgtacccaggggtctttgagacagtggagatgttgccctccaaggcaggcatctggagggtggagtgcttgattggggagcacttgcatgctggcatgtccaccttgttcttggtctactccaacaagtgccagacccccttgggcatggcctctggccacatcagggacttccagatcactgcctcaggccagtatggccagtgggcccccaagttggccaggttgcactacagtggctccatcaatgcctggtccaccaaggagcccttctcctggatcaaggtggacttgttggcccccatgatcatccatggcatcaagacccagggggccaggcagaagttctcctccttgtacatctcccagttcatcatcatgtactccttggatggcaagaagtggcagacctacaggggcaactccacaggcaccttgatggtcttctttggcaatgtggactcctctggcatcaagcacaacatcttcaacccccccatcattgccaggtacatcaggttgcaccccacccactactccatcaggtccaccttgaggatggagttgatgggctgtgacttgaactcctgctccatgcccttgggcatggagtccaaggccatctcagatgcccagatcactgcctcctcctacttcaccaacatgtttgccacctggtccccctccaaggccaggttgcacttgcagggcaggtccaatgcctggaggccccaggtcaacaaccccaaggagtggttgcaggtggacttccagaagaccatgaaggtcacaggggtcaccacccagggggtcaagtccttgttgacctccatgtatgtcaaggagttcttgatctcctcctcccaggatggccaccagtggaccttgttcttccagaatggcaaggtcaaggtcttccagggcaaccaggactccttcacccctgtggtcaactccttggacccccccttgttgaccaggtacttgaggatccacccccagtcctgggtccaccagattgccttgaggatggaggtcttgggctgtgaggcccaggacttgtactgatttaaatctcgagccgggcggagtgtgttagtctctccagagggaggctggttccccagggaagcagagcctgtgtgcgggcagcagctgtgtgcgggcctgggggttgttaagtgcaattatttttaataaaaggggcatttggaaaaaaaaaaaaaaggtagcagtcgacagatgaattctgcagatctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaaaaaagagaaatgttctggcacctgcacttgcactggggacagcctattttgctagtttgttttgtttcgttttgttttgatggagagcgtatgttgtttaaacgcggccgcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 29 - DNA, 6314 bp, synthetic construct
  source 1..6314: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacggcgcgccacgcgttctaggagttaatttttaaaaagcagtcaaaagtccaagtgcccttgcgagcatttactctctctgtttgctctggttaataatctcaggagcacaaacattccttactagttctaggagttaatttttaaaaagcagtcaaaagtccaagtgcccttgcgagcatttactctctctgtttgctctggttaataatctcaggagcacaaacattccttactagttctagagcggccgccagtgtgctggaattcggcttttttagggctggaagctacctttgacatcatttcctctgcgaatgcatgtataatttctacagaacctattagaaaggatcacccagcctctgcttttgtacaactttcccttaaaaaactgccaatcccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactgagagtgctctagttctgcaatacaggacatgctataaaaatggaaagatgttgctttctgagagatcaggtggattcttgggcatttgcttaagcttggtaccgccaccatgcagatagagctgagcacctgcttcttcctgtgcctgctgagattctgcttctcagccaccagaagatactacctgggggctgtagagctgagctgggactacatgcagtcagacctgggtgagctgccagtggatgccagattcccccccagagtgcccaagagcttccccttcaacacctctgtggtgtacaagaagaccctgtttgtggagttcactgatcacctgttcaacatagctaagcccagacccccctggatgggcctgctgggccccaccatccaggctgaggtgtatgatacagtggtgatcaccctgaagaacatggccagccaccctgtgagcctgcatgcagtgggtgtgagctactggaaggcctctgagggtgctgaatatgatgaccagaccagccagagagagaaggaggatgataaggtgttccctgggggcagccacacctatgtgtggcaggtgctgaaggagaatggacccatggccagtgaccccctgtgcctgacctacagctacctgagccatgtagacctggtgaaggacctgaactctggactgattggggcactgctggtgtgcagagagggcagcctggccaaggagaagacccagaccctgcacaagttcatcctgctgtttgctgtgtttgatgagggcaagagctggcactctgaaaccaagaacagcctgatgcaggacagagatgctgcctcagcaagagcctggcccaagatgcacactgtgaatggctatgttaacagaagcctgcctgggctgattgggtgccacagaaagagtgtgtactggcatgtaataggaatgggcaccaccccagaggtgcacagcatcttcctggagggccacaccttcctggtgagaaaccacagacaggccagcctggagatcagccccatcaccttcctgactgctcagaccctgctgatggacctgggccagttcctgctgttctgccacatcagcagccaccagcatgatggaatggaggcctatgtaaaggtggacagctgccctgaggagccccagctgcagatgaagaacaatgaggaggcagaggactatgatgatgacctgactgacagtgagatggatgtggtgagatttgatgatgataacagccccagcttcatccagatcagatctgtagccaagaagcaccccaagacctgggtgcactacatagctgcagaagaggaggactgggactatgcccccctggtgctggcccctgatgatagaagctacaagagccagtacctgaacaatggtccccagagaatagggagaaagtacaagaaggtgagattcatggcctacactgatgagaccttcaagaccagagaggccatccagcatgagtcaggcatcctgggccccctgctgtatggagaagtgggtgacaccctgctgatcatcttcaagaaccaggccagcagaccctacaacatctacccccatggcatcactgatgtcagacccctgtacagcagaagactgcccaagggtgtgaagcacctgaaggacttccccatcctgccaggtgagatcttcaagtacaagtggacagtgactgtagaggatgggcccaccaagtcagaccccagatgcctgaccagatactacagcagctttgtgaacatggagagagacctggccagtggcctgattggccccctgctgatctgctacaaggagtcagttgaccagcagggcaaccagatcatgtctgacaagagaaatgtcatcctgttcagtgtgtttgatgagaacagaagctggtacctgactgagaacatccagagattcctgcccaacccagcaggagttcagctggaggaccctgagttccaggccagcaacatcatgcacagcatcaatggctatgtttttgacagcctgcagctgtcagtgtgcctgcatgaagtggcctactggtacatcctgagcattggtgcccagacagatttcctgagtgtcttcttctcaggctacaccttcaagcacaagatggtgtatgaggacaccctgaccctgttccccttctctggggaaacagtgttcatgagcatggagaaccctggtctgtggatcctgggctgccacaactcagacttcagaaacagaggcatgacagcactgctgaaggtgagcagctgtgacaagaacacaggtgactactatgaagacagctatgaggacatctcagcctacctgctgagcaagaacaatgccattgagcccagaagcttcagccagaacccccctgtgctgaagagacaccagagagagatcaccagaaccaccctgcagtcagaccaggaggagattgactatgatgacaccatcagtgtagaaatgaagaaggaggactttgacatctatgatgaggatgaaaaccagagccccagaagcttccagaagaagaccagacactacttcatagctgcagttgagagactgtgggactatggaatgagcagcagcccccatgtgctgagaaacagagcccagtctggctcagttccccagttcaagaaggtggtgttccaggagttcacagatgggagcttcacccagcccctgtacagaggggagctgaatgagcacctgggcctgctgggcccctacatcagagcagaggtggaggacaacatcatggtgaccttcagaaaccaggccagcagaccctacagcttctacagcagcctgatcagctatgaagaggaccagagacagggagcagaacccagaaagaactttgttaagcccaatgaaaccaagacctacttctggaaggtgcagcaccacatggcccccaccaaggatgaatttgactgcaaggcctgggcctacttctctgatgttgacctggagaaggatgtgcactcaggcctgataggccccctgctggtgtgccacaccaacaccctgaacccagctcatgggagacaggtgactgttcaggagtttgccctgttcttcaccatctttgatgagaccaagagctggtacttcacagagaacatggagagaaactgcagagccccctgcaacatccagatggaggaccccaccttcaaggagaactacagattccatgccatcaatggttacatcatggacaccctgcctgggctggtgatggcccaggaccagagaatcagatggtacctgctgagcatgggcagcaatgaaaacatccacagcatccacttctctggccatgtgttcactgtcagaaagaaggaggagtacaagatggccctgtacaacctgtaccctggagtatttgagactgtggagatgctgcccagcaaggcaggtatctggagagtggagtgcctgattggggagcacctgcatgcaggtatgagcaccctgttcctggtgtacagcaacaagtgccagacccccctgggcatggcctctggacacatcagagacttccagatcactgccagtggccagtatggccagtgggcccccaagctggccagactgcactactcagggagcatcaatgcctggagcaccaaggagcccttcagctggatcaaggtggacctgctggcccccatgatcatccatggtatcaagacccagggtgccagacagaagttcagcagcctgtacatcagccagttcatcatcatgtacagcctggatggcaagaagtggcagacctacagaggcaacagcacaggcaccctgatggtgttctttggcaatgtagacagctctggcatcaagcacaacatcttcaacccccccatcatagctagatacatcagactgcaccccacccactacagcatcagaagcaccctgagaatggagctgatgggctgtgacctgaacagctgcagcatgcccctgggcatggagagcaaggccatctcagatgctcagatcactgccagcagctacttcaccaacatgtttgccacctggagccccagcaaggccagactgcacctgcagggcagaagcaatgcctggagaccccaggtgaacaaccccaaggagtggctgcaggtggacttccagaagaccatgaaggtgacaggagtgaccacccagggggtgaagagcctgctgaccagcatgtatgtgaaggagttcctgatcagcagcagccaggatggacaccagtggaccctgttcttccagaatggaaaggtgaaggtgttccagggcaaccaggacagcttcacccctgtggtgaacagcctggacccccccctgctgaccagatacctgagaatccacccccagagctgggtgcaccagattgccctgagaatggaggtgctgggctgtgaggcccaggacctgtactgatttaaatctcgagccgggcggagtgtgttagtctctccagagggaggctggttccccagggaagcagagcctgtgtgcgggcagcagctgtgtgcgggcctgggggttgttaagtgcaattatttttaataaaaggggcatttggaaaaaaaaaaaaaaggtagcagtcgacagatgaattctgcagatctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaaaaaagagaaatgttctggcacctgcacttgcactggggacagcctattttgctagtttgttttgtttcgttttgttttgatggagagcgtatgttgtttaaacgcggccgcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 30 - DNA, 6314 bp, synthetic construct
  source 1..6314: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacggcgcgccacgcgttctaggagttaatttttaaaaagcagtcaaaagtccaagtgcccttgcgagcatttactctctctgtttgctctggttaataatctcaggagcacaaacattccttactagttctaggagttaatttttaaaaagcagtcaaaagtccaagtgcccttgcgagcatttactctctctgtttgctctggttaataatctcaggagcacaaacattccttactagttctagagcggccgccagtgtgctggaattcggcttttttagggctggaagctacctttgacatcatttcctctgcgaatgcatgtataatttctacagaacctattagaaaggatcacccagcctctgcttttgtacaactttcccttaaaaaactgccaatcccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactgagagtgctctagttctgcaatacaggacatgctataaaaatggaaagatgttgctttctgagagatcaggtggattcttgggcatttgcttaagcttggtaccgccaccatgcagattgagctgagcacctgcttcttcctgtgcctgctgagattctgcttctctgctaccagaagatactacctgggagctgtggagctgagctgggactacatgcagtcagacctgggtgaactgcctgtggatgccagattcccccccagagtgcccaagagcttccccttcaacaccagtgttgtctacaagaagaccctgtttgtggagttcacagaccacctgttcaacattgccaagcccagacccccctggatgggcctgctgggccccaccatccaggctgaagtgtatgatacagtggtgatcaccctgaagaacatggccagccacccagtgagcctgcatgcagtgggtgtgagctactggaaggcctctgaaggtgcagagtatgatgaccagaccagccagagagagaaggaggatgacaaggtgttccctgggggcagccacacctatgtgtggcaggtgctgaaggagaatggccccatggccagtgatcccctgtgcctgacctacagctacctgagccatgttgacctggtgaaggacctgaactcaggtctgattggagccctgctggtgtgcagagagggcagcctggccaaggagaagacccagaccctgcacaagttcatcctgctgtttgctgtctttgatgaaggcaagagctggcacagtgagaccaagaacagcctgatgcaggacagagatgcagcctctgccagagcctggcccaagatgcacacagtaaatggctatgtgaacagaagcctgccagggctgattggctgccacagaaagtctgtttactggcatgtgattgggatgggcaccacccctgaagtgcacagcatcttcctggagggccacaccttcctggtgagaaaccacagacaggccagcctggagatcagccccatcaccttcctgacagcacagaccctgctgatggacctgggccagttcctgctgttctgccacatcagcagccaccagcatgatgggatggaggcctatgtgaaggtggacagctgcccagaagagccccagctgcagatgaagaacaatgaggaggcagaggactatgatgatgacctgactgactctgagatggatgttgtgagatttgatgatgacaacagccccagcttcatccagatcagaagtgttgccaagaagcaccccaagacctgggtgcactacattgcagctgaggaggaggactgggactatgcccccctggtgctggccccagatgacagaagctacaagagccagtacctgaacaatggcccccagagaattggtagaaagtacaagaaggtgagattcatggcctacacagatgagaccttcaagaccagagaggccatccagcatgagtcaggaatcctgggccccctgctgtatggggaggtgggggataccctgctgatcatcttcaagaaccaggccagcagaccctacaacatctacccccatggcatcacagatgtgagacccctgtacagcagaagactgcccaagggagtgaagcacctgaaggacttccccatcctgcctggtgagatcttcaagtacaagtggacagttactgtggaagatggtcccaccaagtcagaccccagatgcctgaccagatactacagcagctttgtgaacatggagagagacctggcctctgggctgattgggcccctgctgatctgctacaaggagagtgtggaccagcagggcaaccagatcatgtctgataagagaaatgtgatcctgttctctgtatttgatgaaaacagaagctggtacctgactgagaacatccagagattcctgcccaacccagctggagtccagctggaggacccagagttccaggccagcaacatcatgcacagcatcaatggatatgtgtttgacagcctgcagctgtctgtatgcctgcatgaggtggcctactggtacatcctgagcattggggcccagactgacttcctgtctgtgttcttctcaggatacaccttcaagcacaagatggtgtatgaggacaccctgaccctgttccccttctcaggggaaactgtgttcatgagcatggagaaccctggcctgtggatcctgggctgccacaactctgacttcagaaacagaggcatgactgccctgctgaaggtgagcagctgtgacaagaacactggggattactatgaggacagctatgaggacatctcagcctacctgctgagcaagaacaatgccattgaacccagaagcttcagccagaacccccctgtcctgaagagacaccagagagagatcaccagaaccaccctgcagtctgatcaggaggagattgactatgatgataccatctcagtggagatgaagaaggaggactttgacatctatgatgaagatgaaaaccagagccccagaagcttccagaagaagaccagacactacttcattgctgctgtggagagactgtgggactatgggatgagcagcagcccccatgtgctgagaaacagagcccagtcaggcagtgtgccccagttcaagaaggtggtgttccaggagttcacagatggaagcttcacccagcccctgtacagaggtgagctgaatgaacacctgggcctgctgggcccctacatcagagctgaagtggaggacaacatcatggtgaccttcagaaaccaggccagcagaccctacagcttctacagcagcctgatcagctatgaggaggaccagagacagggagcagagcccagaaagaactttgtcaagcccaatgaaaccaagacctacttctggaaggtgcagcaccacatggcccccaccaaggatgaatttgactgcaaggcctgggcctacttctctgatgtagacctggagaaggatgtgcactctgggctgattggccccctgctggtgtgccacaccaacaccctgaacccagcccatggcagacaggtgactgtgcaggagtttgccctgttcttcaccatctttgatgaaaccaagagctggtacttcactgagaacatggagagaaactgcagagccccctgcaacatccagatggaggaccccaccttcaaggagaactacagattccatgctatcaatggatacatcatggacaccctgcctgggctggtgatggcccaggaccagagaatcagatggtacctgctgagcatgggcagcaatgagaacatccacagcatccacttctcaggacatgtcttcacagtaagaaagaaggaggagtacaagatggccctgtacaacctgtacccaggggtctttgaaactgtagagatgctgcccagcaaggcaggcatctggagagtggagtgcctgattggagagcacctgcatgctggtatgagcaccctgttcctggtgtacagcaacaagtgccagacccccctgggcatggcctctggccacatcagagacttccagatcacagccagtggccagtatgggcagtgggcccccaagctggccagactgcactactctggcagcatcaatgcctggagcaccaaggagcccttcagctggatcaaggtggacctgctggcccccatgatcatccatgggatcaagacccagggtgccagacagaagttcagcagcctgtacatcagccagttcatcatcatgtacagcctggatggcaagaagtggcagacctacagaggcaacagcacaggtaccctgatggtgttctttggcaatgtggacagctcaggcatcaagcacaacatcttcaacccccccatcattgccagatacatcagactgcaccccacccactacagcatcagaagcaccctgagaatggagctgatgggctgtgacctgaacagctgcagcatgcccctgggcatggagagcaaggccatctctgatgcccagatcacagctagcagctacttcaccaacatgtttgccacctggagccccagcaaggccagactgcacctgcagggcagaagcaatgcttggagaccccaggtgaacaaccccaaggagtggctgcaggtggacttccagaagaccatgaaggtgacaggtgtgaccacccagggggttaagagcctgctgaccagcatgtatgtgaaggagttcctgatcagcagcagccaggatggccaccagtggaccctgttcttccagaatggcaaggtgaaggtgttccagggcaaccaggacagcttcacccctgttgtgaacagcctggacccccccctgctgaccagatacctgagaatccacccccagagctgggtgcaccagattgccctgagaatggaggtgctgggctgtgaggcccaggacctgtactgatttaaatctcgagccgggcggagtgtgttagtctctccagagggaggctggttccccagggaagcagagcctgtgtgcgggcagcagctgtgtgcgggcctgggggttgttaagtgcaattatttttaataaaaggggcatttggaaaaaaaaaaaaaaggtagcagtcgacagatgaattctgcagatctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaaaaaagagaaatgttctggcacctgcacttgcactggggacagcctattttgctagtttgttttgtttcgttttgttttgatggagagcgtatgttgtttaaacgcggccgcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 31 - DNA, 6314 bp, synthetic construct
  source 1..6314: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacggcgcgccacgcgttctaggagttaatttttaaaaagcagtcaaaagtccaagtgcccttgcgagcatttactctctctgtttgctctggttaataatctcaggagcacaaacattccttactagttctaggagttaatttttaaaaagcagtcaaaagtccaagtgcccttgcgagcatttactctctctgtttgctctggttaataatctcaggagcacaaacattccttactagttctagagcggccgccagtgtgctggaattcggcttttttagggctggaagctacctttgacatcatttcctctgcgaatgcatgtataatttctacagaacctattagaaaggatcacccagcctctgcttttgtacaactttcccttaaaaaactgccaatcccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactgagagtgctctagttctgcaatacaggacatgctataaaaatggaaagatgttgctttctgagagatcaggtggattcttgggcatttgcttaagcttggtaccgccaccatgcagatagaactgagcacctgcttcttcctgtgcctgctgagattctgcttctcagccaccagaagatactacctgggggcagttgagctgagctgggactacatgcagagtgatctgggggagctgccagtggatgcaagattcccccccagagtgcccaagagcttccccttcaacaccagtgtggtctacaagaagaccctgtttgttgagttcacagaccacctgttcaacattgctaagcccagacccccctggatgggcctgctgggccccaccatccaggcagaggtgtatgatacagttgtgatcaccctgaagaacatggccagccacccagttagcctgcatgcagttggagtgagctactggaaggcctcagagggagctgagtatgatgaccagaccagccagagagagaaggaggatgataaggtgttcccaggggggagccacacctatgtgtggcaggtgctgaaggagaatggacccatggccagtgaccccctgtgcctgacctacagctacctgagccatgtggacctggtgaaggacctgaactcaggactgataggtgctctgctggtgtgcagagagggcagcctggccaaggagaagacccagaccctgcacaagttcatcctgctgtttgcagtttttgatgaaggcaagagctggcactcagagaccaagaacagcctgatgcaggacagagatgcagcttctgctagagcctggcccaagatgcacactgtcaatggctatgtaaacagaagcctgcctggcctgattggctgccacagaaagtctgtctactggcatgttataggtatgggcaccacccctgaggtgcacagcatcttcctggagggccacaccttcctggtgagaaaccacagacaggccagcctggagatcagccccatcaccttcctgactgcccagaccctgctgatggacctgggccagttcctgctgttctgccacatcagcagccaccagcatgatggcatggaggcctatgtcaaggtggacagctgcccagaagagccccagctgcagatgaagaacaatgaggaggctgaggactatgatgatgacctgactgacagtgagatggatgtagtgagatttgatgatgacaacagccccagcttcatccagatcagaagtgtagccaagaagcaccccaagacctgggtgcactacattgctgcagaggaggaggactgggactatgcccccctggtgctggccccagatgacagaagctacaagagccagtacctgaacaatggtccccagagaattggcagaaagtacaagaaggtgagattcatggcctacacagatgagaccttcaagaccagagaggccatccagcatgagtcaggcatcctgggccccctgctgtatggagaggtgggggacaccctgctgatcatcttcaagaaccaggccagcagaccctacaacatctacccccatggaatcacagatgtgagacccctgtacagcagaagactgcccaagggggtgaagcacctgaaggacttccccatcctgcctggagagatcttcaagtacaagtggactgtgacagtagaggatggacccaccaagtcagaccccagatgcctgaccagatactacagcagctttgtaaacatggagagagacctggccagtggcctgataggccccctgctgatctgctacaaggagtcagtggaccagcagggcaaccagatcatgtcagacaagagaaatgtgatcctgttctctgtatttgatgagaacagaagctggtacctgactgaaaacatccagagattcctgcccaaccctgctggagtacagctggaggaccctgagttccaggccagcaacatcatgcacagcatcaatggttatgtgtttgacagcctgcagctgtctgtgtgcctgcatgaagtggcctactggtacatcctgagcataggtgcccagactgacttcctgtctgtgttcttcagtggctacaccttcaagcacaagatggtgtatgaagacaccctgaccctgttccccttctctggggagacagtattcatgagcatggagaacccaggactgtggatcctgggctgccacaacagtgacttcagaaacagaggcatgactgctctgctgaaggtgagcagctgtgataagaacacaggtgactactatgaggacagctatgaagacatcagtgcttacctgctgagcaagaacaatgctatagaacccagaagcttcagccagaaccccccagttctgaagagacaccagagagagatcaccagaaccaccctgcagtcagaccaggaggagatagactatgatgataccatctcagtagagatgaagaaggaggactttgacatctatgatgaggatgaaaaccagagccccagaagcttccagaagaagaccagacactacttcatagctgctgtagagagactgtgggactatggaatgagcagcagcccccatgtgctgagaaacagagcccagtctgggagtgtgccccagttcaagaaggtggtgttccaggagttcacagatgggagcttcacccagcccctgtacagaggggagctgaatgaacacctgggcctgctgggcccctacatcagagcagaggtggaggacaacatcatggtgaccttcagaaaccaggccagcagaccctacagcttctacagcagcctgatcagctatgaggaggaccagagacagggagctgagcccagaaagaactttgttaagcccaatgaaaccaagacctacttctggaaggtgcagcaccacatggcccccaccaaggatgagtttgactgcaaggcctgggcctacttctctgatgttgacctggagaaggatgtgcactctggactgattgggcccctgctggtgtgccacaccaacaccctgaaccctgcccatgggagacaggtgacagtacaggagtttgctctgttcttcaccatctttgatgagaccaagagctggtacttcacagaaaacatggagagaaactgcagagccccctgcaacatccagatggaggaccccaccttcaaggagaactacagattccatgctatcaatggctacatcatggacaccctgcctggcctggtgatggcccaggaccagagaatcagatggtacctgctgagcatgggcagcaatgaaaacatccacagcatccacttctctggtcatgttttcacagttagaaagaaggaggagtacaagatggccctgtacaacctgtaccctggggtgtttgagactgtggagatgctgcccagcaaggctggaatctggagagtggagtgcctgattggagaacacctgcatgcagggatgagcaccctgttcctggtgtacagcaacaagtgccagacccccctgggcatggccagtggtcacatcagagacttccagatcacagcctcaggccagtatggacagtgggcccccaagctggccagactgcactacagtggtagcatcaatgcctggagcaccaaggagcccttcagctggatcaaggtggacctgctggcccccatgatcatccatggaatcaagacccagggggccagacagaagttcagcagcctgtacatcagccagttcatcatcatgtacagcctggatggcaagaagtggcagacctacagaggcaacagcactggtaccctgatggtgttctttggcaatgtggacagctctgggatcaagcacaacatcttcaacccccccatcatagccagatacatcagactgcaccccacccactacagcatcagaagcaccctgagaatggagctgatgggctgtgacctgaacagctgcagcatgcccctgggcatggagagcaaggccatcagtgatgctcagatcacagcaagcagctacttcaccaacatgtttgccacctggagccccagcaaggccagactgcacctgcagggcagaagcaatgcttggagaccccaggtgaacaaccccaaggagtggctgcaggtggacttccagaagaccatgaaggtgactggggtgaccacccagggggtgaagagcctgctgaccagcatgtatgtcaaggagttcctgatcagcagcagccaggatgggcaccagtggaccctgttcttccagaatgggaaggtgaaggtgttccagggcaaccaggacagcttcaccccagtagtgaacagcctggacccccccctgctgaccagatacctgagaatccacccccagagctgggtgcaccagatagccctgagaatggaggtgctgggctgtgaggcccaggacctgtactgatttaaatctcgagccgggcggagtgtgttagtctctccagagggaggctggttccccagggaagcagagcctgtgtgcgggcagcagctgtgtgcgggcctgggggttgttaagtgcaattatttttaataaaaggggcatttggaaaaaaaaaaaaaaggtagcagtcgacagatgaattctgcagatctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaaaaaagagaaatgttctggcacctgcacttgcactggggacagcctattttgctagtttgttttgtttcgttttgttttgatggagagcgtatgttgtttaaacgcggccgcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 32 - DNA, 6314 bp, synthetic construct
  source 1..6314: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacggcgcgccacgcgttctaggagttaatttttaaaaagcagtcaaaagtccaagtgcccttgcgagcatttactctctctgtttgctctggttaataatctcaggagcacaaacattccttactagttctaggagttaatttttaaaaagcagtcaaaagtccaagtgcccttgcgagcatttactctctctgtttgctctggttaataatctcaggagcacaaacattccttactagttctagagcggccgccagtgtgctggaattcggcttttttagggctggaagctacctttgacatcatttcctctgcgaatgcatgtataatttctacagaacctattagaaaggatcacccagcctctgcttttgtacaactttcccttaaaaaactgccaatcccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactgagagtgctctagttctgcaatacaggacatgctataaaaatggaaagatgttgctttctgagagatcaggtggattcttgggcatttgcttaagcttggtaccgccaccatgcagatagagctgagcacctgcttcttcctgtgcctgctgagattctgcttctcagccaccagaagatactacctgggtgcagtggagctgagctgggactacatgcagagtgacctgggggaactgcctgtggatgccagattcccccccagagtgcccaagagcttccccttcaacacctctgttgtttacaagaagaccctgtttgtagagttcactgaccacctgttcaacatagccaagcccagacccccctggatgggcctgctgggccccaccatccaggcagaggtgtatgatacagtggtgatcaccctgaagaacatggccagccaccctgttagcctgcatgcagtgggggtgagctactggaaggccagtgagggagcagagtatgatgatcagaccagccagagagagaaggaggatgacaaggtgttccctgggggcagccacacctatgtatggcaggtgctgaaggagaatggccccatggcctcagatcccctgtgcctgacctacagctacctgagccatgtggacctggtgaaggacctgaacagtggtctgattggggccctgctggtgtgcagagagggcagcctggccaaggagaagacccagaccctgcacaagttcatcctgctgtttgctgtctttgatgagggcaagagctggcactcagagaccaagaacagcctgatgcaggacagagatgctgcctctgccagagcctggcccaagatgcacactgtgaatgggtatgtgaacagaagcctgccaggtctgataggctgccacagaaagtcagtatactggcatgtgattggcatgggcaccaccccagaggtgcacagcatcttcctggagggccacaccttcctggtgagaaaccacagacaggccagcctggagatcagccccatcaccttcctgactgcccagaccctgctgatggacctgggccagttcctgctgttctgccacatcagcagccaccagcatgatggcatggaggcctatgttaaggtggacagctgcccagaggagccccagctgcagatgaagaacaatgaagaggctgaggactatgatgatgatctgacagactcagaaatggatgtagtcagatttgatgatgataacagccccagcttcatccagatcagatctgtagccaagaagcaccccaagacctgggtgcactacatagcagcagaggaggaggactgggactatgcacccctggtgctggcccctgatgacagaagctacaagagccagtacctgaacaatggcccccagagaataggcagaaagtacaagaaggtgagattcatggcctacactgatgagaccttcaagaccagagaggccatccagcatgagtctggaatcctgggccccctgctgtatggtgaggtgggtgacaccctgctgatcatcttcaagaaccaggccagcagaccctacaacatctacccccatggcatcactgatgtgagacccctgtacagcagaagactgcccaagggggtgaagcacctgaaggacttccccatcctgccaggtgagatcttcaagtacaagtggactgtgacagtggaggatggccccaccaagagtgaccccagatgcctgaccagatactacagcagctttgtcaacatggagagagacctggccagtgggctgatagggcccctgctgatctgctacaaggagtcagtggaccagcagggcaaccagatcatgagtgataagagaaatgtgatcctgttctcagtttttgatgaaaacagaagctggtacctgacagaaaacatccagagattcctgcccaacccagcaggggtacagctggaggaccctgaattccaggccagcaacatcatgcacagcatcaatggctatgtatttgacagcctgcagctgtctgtttgcctgcatgaggtggcctactggtacatcctgagcataggagcccagacagacttcctgagtgtgttcttcagtggctacaccttcaagcacaagatggtgtatgaggacaccctgaccctgttccccttctcaggggagactgttttcatgagcatggagaaccctggactgtggatcctgggctgccacaactctgacttcagaaacagaggcatgactgccctgctgaaggtgagcagctgtgataagaacactggagactactatgaggacagctatgaggacatctcagcctacctgctgagcaagaacaatgccattgaacccagaagcttcagccagaacccccctgtactgaagagacaccagagagagatcaccagaaccaccctgcagagtgaccaggaggagatagactatgatgataccatctctgtggagatgaagaaggaggactttgatatctatgatgaggatgagaaccagagccccagaagcttccagaagaagaccagacactacttcatagcagcagtggagagactgtgggactatggcatgagcagcagcccccatgtgctgagaaacagagcccagtcaggcagtgttccccagttcaagaaggtggtgttccaggagttcactgatggtagcttcacccagcccctgtacagaggagagctgaatgagcacctgggcctgctgggcccctacatcagagctgaggtggaggacaacatcatggtgaccttcagaaaccaggccagcagaccctacagcttctacagcagcctgatcagctatgaggaggaccagagacagggagcagagcccagaaagaactttgttaagcccaatgaaaccaagacctacttctggaaggtgcagcaccacatggcccccaccaaggatgagtttgattgcaaggcctgggcctacttcagtgatgtagacctggagaaggatgtgcactcaggcctgattggacccctgctggtgtgccacaccaacaccctgaaccctgcacatggaagacaggtgacagtgcaggagtttgccctgttcttcaccatctttgatgagaccaagagctggtacttcacagagaacatggagagaaactgcagagccccctgcaacatccagatggaggaccccaccttcaaggagaactacagattccatgctatcaatggctacatcatggacaccctgcctggcctggtgatggcccaggaccagagaatcagatggtacctgctgagcatgggcagcaatgagaacatccacagcatccacttcagtgggcatgtgttcacagtgagaaagaaggaggagtacaagatggccctgtacaacctgtaccctggagtgtttgagactgtggagatgctgcccagcaaggcaggcatctggagagtggagtgcctgataggagaacacctgcatgctggcatgagcaccctgttcctggtgtacagcaacaagtgccagacccccctgggcatggccagtggacacatcagagacttccagatcacagcctctggacagtatggacagtgggcccccaagctggccagactgcactactcaggaagcatcaatgcatggagcaccaaggagcccttcagctggatcaaggtggacctgctggcccccatgatcatccatggcatcaagacccagggggcaagacagaagttcagcagcctgtacatcagccagttcatcatcatgtacagcctggatggcaagaagtggcagacctacagaggcaacagcactggaaccctgatggtgttctttgggaatgtggacagctcagggatcaagcacaacatcttcaacccccccatcattgctagatacatcagactgcaccccacccactacagcatcagaagcaccctgagaatggagctgatgggctgtgatctgaacagctgcagcatgcccctgggcatggagagcaaggccatctcagatgcccagatcactgccagcagctacttcaccaacatgtttgccacctggagccccagcaaggccagactgcacctgcagggcagaagcaatgcttggagaccccaggtgaacaaccccaaggagtggctgcaggtggacttccagaagaccatgaaggtgactggagtaaccacccagggggtgaagagcctgctgaccagcatgtatgtgaaggagttcctgatcagcagcagccaggatggtcaccagtggaccctgttcttccagaatggcaaggtgaaggtgttccagggcaaccaggacagcttcacccctgttgtgaacagcctggacccccccctgctgaccagatacctgagaatccacccccagagctgggtgcaccagattgccctgagaatggaggtgctgggctgtgaggcccaggacctgtactgatttaaatctcgagccgggcggagtgtgttagtctctccagagggaggctggttccccagggaagcagagcctgtgtgcgggcagcagctgtgtgcgggcctgggggttgttaagtgcaattatttttaataaaaggggcatttggaaaaaaaaaaaaaaggtagcagtcgacagatgaattctgcagatctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaaaaaagagaaatgttctggcacctgcacttgcactggggacagcctattttgctagtttgttttgtttcgttttgttttgatggagagcgtatgttgtttaaacgcggccgcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 33 - DNA, 6314 bp, synthetic construct
  source 1..6314: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacggcgcgccacgcgttctaggagttaatttttaaaaagcagtcaaaagtccaagtgcccttgcgagcatttactctctctgtttgctctggttaataatctcaggagcacaaacattccttactagttctaggagttaatttttaaaaagcagtcaaaagtccaagtgcccttgcgagcatttactctctctgtttgctctggttaataatctcaggagcacaaacattccttactagttctagagcggccgccagtgtgctggaattcggcttttttagggctggaagctacctttgacatcatttcctctgcgaatgcatgtataatttctacagaacctattagaaaggatcacccagcctctgcttttgtacaactttcccttaaaaaactgccaatcccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactgagagtgctctagttctgcaatacaggacatgctataaaaatggaaagatgttgctttctgagagatcaggtggattcttgggcatttgcttaagcttggtaccgccaccatgcagatagagctgagcacctgcttcttcctgtgcctgctgagattctgcttctctgccaccagaagatactacctgggtgcagtggagctgagctgggactacatgcagtcagacctgggagagctgccagtggatgctagattcccccccagagtgcccaagagcttccccttcaacacctcagtggtgtacaagaagaccctgtttgtagagttcactgaccacctgttcaacattgccaagcccagacccccctggatgggcctgctgggccccaccatccaggctgaggtgtatgatacagtggtaatcaccctgaagaacatggccagccacccagtgagcctgcatgctgttggagttagctactggaaggcctctgagggtgctgagtatgatgatcagaccagccagagagagaaggaggatgataaggtgttcccagggggaagccacacctatgtatggcaggtgctgaaggagaatgggcccatggccagtgaccccctgtgcctgacctacagctacctgagccatgtggatctggtgaaggacctgaactcaggtctgataggggccctgctggtgtgcagagagggcagcctggccaaggagaagacccagaccctgcacaagttcatcctgctgtttgctgtatttgatgagggcaagagctggcacagtgaaaccaagaacagcctgatgcaggacagagatgctgcctctgccagagcctggcccaagatgcacacagtgaatggctatgtgaacagaagcctgcctggtctgatagggtgccacagaaagtcagtgtactggcatgtcattggcatgggcaccaccccagaggtgcacagcatcttcctggagggccacaccttcctggtgagaaaccacagacaggccagcctggagatcagccccatcaccttcctgacagctcagaccctgctgatggacctgggccagttcctgctgttctgccacatcagcagccaccagcatgatggcatggaggcctatgttaaggtggacagctgcccagaggagccccagctgcagatgaagaacaatgaggaggcagaggactatgatgatgatctgacagacagtgagatggatgtggtgagatttgatgatgacaacagccccagcttcatccagatcagatctgtggccaagaagcaccccaagacctgggtgcactacattgcagctgaagaggaggactgggactatgcccccctggtgctggccccagatgatagaagctacaagagccagtacctgaacaatggcccccagagaattggaagaaagtacaagaaggtgagattcatggcctacactgatgaaaccttcaagaccagagaggccatccagcatgagtcaggcatcctgggccccctgctgtatggtgaggtgggtgataccctgctgatcatcttcaagaaccaggccagcagaccctacaacatctacccccatggcatcactgatgtaagacccctgtacagcagaagactgcccaagggtgtaaagcacctgaaggacttccccatcctgcctggggaaatcttcaagtacaagtggactgtgactgtagaggatggtcccaccaagagtgaccccagatgcctgaccagatactacagcagctttgtgaacatggagagagacctggcctctggtctgataggtcccctgctgatctgctacaaggagtcagtagaccagcagggcaaccagatcatgtctgacaagagaaatgtgatcctgttcagtgtgtttgatgagaacagaagctggtacctgacagaaaacatccagagattcctgcccaacccagctggagtgcagctggaggaccctgagttccaggccagcaacatcatgcacagcatcaatgggtatgtctttgacagcctgcagctgagtgtgtgcctgcatgaggtggcctactggtacatcctgagcattggtgctcagacagatttcctgtctgtgttcttcagtggttacaccttcaagcacaagatggtgtatgaagacaccctgaccctgttccccttcagtggagagacagtattcatgagcatggagaaccctggcctgtggatcctgggctgccacaacagtgatttcagaaacagaggcatgactgcactgctgaaggtgagcagctgtgacaagaacactggggattactatgaagacagctatgaggacatcagtgcctacctgctgagcaagaacaatgctattgagcccagaagcttcagccagaaccccccagtcctgaagagacaccagagagagatcaccagaaccaccctgcagagtgaccaggaggagatagactatgatgacaccatctcagtggagatgaagaaggaggactttgacatctatgatgaggatgagaaccagagccccagaagcttccagaagaagaccagacactacttcattgcagcagtagagagactgtgggactatgggatgagcagcagcccccatgtactgagaaacagagcccagtctggatctgtgccccagttcaagaaggtggtgttccaggagttcactgatggcagcttcacccagcccctgtacagaggagagctgaatgagcacctgggcctgctgggcccctacatcagagcagaggtggaggacaacatcatggtgaccttcagaaaccaggccagcagaccctacagcttctacagcagcctgatcagctatgaggaggaccagagacagggtgctgagcccagaaagaactttgttaagcccaatgagaccaagacctacttctggaaggtgcagcaccacatggcccccaccaaggatgaatttgattgcaaggcctgggcctacttcagtgatgtagacctggagaaggatgtgcacagtggactgattgggcccctgctggtgtgccacaccaacaccctgaacccagcccatggcagacaggtgactgtgcaggagtttgctctgttcttcaccatctttgatgaaaccaagagctggtacttcacagagaacatggagagaaactgcagagccccctgcaacatccagatggaggaccccaccttcaaggagaactacagattccatgctatcaatggctacatcatggacaccctgccaggtctggtgatggcccaggaccagagaatcagatggtacctgctgagcatgggcagcaatgagaacatccacagcatccacttctctggccatgtcttcactgtgagaaagaaggaggagtacaagatggccctgtacaacctgtacccaggtgtctttgagactgtagagatgctgcccagcaaggctggcatctggagagtggagtgcctgattggagagcacctgcatgcaggaatgagcaccctgttcctggtgtacagcaacaagtgccagacccccctgggcatggccagtggccacatcagagacttccagatcacagcctctgggcagtatggacagtgggcccccaagctggccagactgcactactcaggaagcatcaatgcctggagcaccaaggagcccttcagctggatcaaggtggacctgctggcccccatgatcatccatgggatcaagacccagggggccagacagaagttcagcagcctgtacatcagccagttcatcatcatgtacagcctggatggtaagaagtggcagacctacagaggcaacagcacaggcaccctgatggtgttctttggaaatgtagacagctctggcatcaagcacaacatcttcaacccccccatcatagccagatacatcagactgcaccccacccactacagcatcagaagcaccctgagaatggagctgatgggctgtgacctgaacagctgcagcatgcccctgggcatggagagcaaggccatctctgatgctcagatcactgccagcagctacttcaccaacatgtttgcaacctggagccccagcaaggccagactgcacctgcagggcagaagcaatgcctggagaccccaggtgaacaaccccaaggagtggctgcaggtggacttccagaagaccatgaaggtgactggtgtaaccacccagggagtgaagagcctgctgaccagcatgtatgtcaaggagttcctgatcagcagcagccaggatggtcaccagtggaccctgttcttccagaatggaaaggtgaaggtgttccagggcaaccaggacagcttcaccccagttgtgaacagcctggacccccccctgctgaccagatacctgagaatccacccccagagctgggtgcaccagattgccctgagaatggaggtgctgggctgtgaggcccaggacctgtactgatttaaatctcgagccgggcggagtgtgttagtctctccagagggaggctggttccccagggaagcagagcctgtgtgcgggcagcagctgtgtgcgggcctgggggttgttaagtgcaattatttttaataaaaggggcatttggaaaaaaaaaaaaaaggtagcagtcgacagatgaattctgcagatctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaaaaaagagaaatgttctggcacctgcacttgcactggggacagcctattttgctagtttgttttgtttcgttttgttttgatggagagcgtatgttgtttaaacgcggccgcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 34 - DNA, 6314 bp, synthetic construct
  source 1..6314: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacggcgcgccacgcgttctaggagttaatttttaaaaagcagtcaaaagtccaagtgcccttgcgagcatttactctctctgtttgctctggttaataatctcaggagcacaaacattccttactagttctaggagttaatttttaaaaagcagtcaaaagtccaagtgcccttgcgagcatttactctctctgtttgctctggttaataatctcaggagcacaaacattccttactagttctagagcggccgccagtgtgctggaattcggcttttttagggctggaagctacctttgacatcatttcctctgcgaatgcatgtataatttctacagaacctattagaaaggatcacccagcctctgcttttgtacaactttcccttaaaaaactgccaatcccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactgagagtgctctagttctgcaatacaggacatgctataaaaatggaaagatgttgctttctgagagatcaggtggattcttgggcatttgcttaagcttggtaccgccaccatgcagatagagctgagcacctgcttcttcctgtgcctgctgagattctgcttcagtgcaaccagaagatactacctgggtgcagtggagctgagctgggactacatgcagtctgatctgggggagctgccagttgatgccagattcccccccagagtgcccaagagcttccccttcaacaccagtgttgtttacaagaagaccctgtttgtggaattcactgaccacctgttcaacatagccaagcccagacccccctggatgggcctgctgggccccaccatccaggcagaggtgtatgacacagtagtgatcaccctgaagaacatggccagccaccctgtgagcctgcatgcagtaggggtaagctactggaaggcctctgaaggtgctgaatatgatgaccagaccagccagagagagaaggaggatgacaaggtgttcccaggtggaagccacacctatgtctggcaggtgctgaaggagaatggccccatggccagtgaccccctgtgcctgacctacagctacctgagccatgtggacctggtgaaggacctgaactcaggtctgattggagcactgctggtgtgcagagagggcagcctggccaaggagaagacccagaccctgcacaagttcatcctgctgtttgcagtgtttgatgaaggcaagagctggcacagtgagaccaagaacagcctgatgcaggacagagatgcagccagtgccagagcctggcccaagatgcacactgtgaatggttatgtcaacagaagcctgccagggctgataggctgccacagaaagagtgtttactggcatgtaattgggatgggcaccaccccagaggtgcacagcatcttcctggagggccacaccttcctggtgagaaaccacagacaggccagcctggagatcagccccatcaccttcctgacagctcagaccctgctgatggacctgggccagttcctgctgttctgccacatcagcagccaccagcatgatggcatggaggcctatgtaaaggtggacagctgccctgaggagccccagctgcagatgaagaacaatgaggaggcagaggactatgatgatgacctgactgacagtgagatggatgtggtgagatttgatgatgacaacagccccagcttcatccagatcagaagtgttgctaagaagcaccccaagacctgggtgcactacattgcagcagaggaggaggactgggactatgctcccctggtgctggcccctgatgacagaagctacaagagccagtacctgaacaatggcccccagagaattggcagaaagtacaagaaggtgagattcatggcctacactgatgagaccttcaagaccagagaggccatccagcatgaaagtggcatcctgggccccctgctgtatggagaggtgggagacaccctgctgatcatcttcaagaaccaggccagcagaccctacaacatctacccccatggtatcacagatgtgagacccctgtacagcagaagactgcccaagggggtgaagcacctgaaggacttccccatcctgccaggtgaaatcttcaagtacaagtggacagtcacagtggaggatggccccaccaagagtgaccccagatgcctgaccagatactacagcagctttgtgaacatggagagagacctggcctctggcctgataggccccctgctgatctgctacaaggagtcagtggaccagcagggcaaccagatcatgagtgataagagaaatgtgatcctgttctcagtatttgatgagaacagaagctggtacctgactgaaaacatccagagattcctgcccaaccctgcaggggtgcagctggaggaccctgaattccaggccagcaacatcatgcacagcatcaatggctatgtatttgatagcctgcagctgagtgtgtgcctgcatgaggtggcctactggtacatcctgagcataggggcccagacagacttcctgtctgtgttcttctcaggttacaccttcaagcacaagatggtgtatgaagacaccctgaccctgttccccttcagtggggaaacagtattcatgagcatggagaaccctgggctgtggatcctgggctgccacaactcagatttcagaaacagaggcatgactgccctgctgaaggtgagcagctgtgacaagaacactggagactactatgaggacagctatgaggacatcagtgcctacctgctgagcaagaacaatgctatagagcccagaagcttcagccagaaccccccagtcctgaagagacaccagagagagatcaccagaaccaccctgcagtcagatcaggaggagatagactatgatgacaccatcagtgtggagatgaagaaggaggactttgacatctatgatgaggatgagaaccagagccccagaagcttccagaagaagaccagacactacttcatagctgctgtagagagactgtgggactatggcatgagcagcagcccccatgtcctgagaaacagagcccagtctggctctgttccccagttcaagaaggtggtgttccaggagttcacagatggcagcttcacccagcccctgtacagaggggagctgaatgaacacctgggcctgctgggcccctacatcagagctgaagtggaggacaacatcatggtgaccttcagaaaccaggccagcagaccctacagcttctacagcagcctgatcagctatgaggaggaccagagacagggtgcagaacccagaaagaactttgttaagcccaatgagaccaagacctacttctggaaggtgcagcaccacatggcccccaccaaggatgaatttgactgcaaggcctgggcctacttctcagatgttgacctggagaaggatgtccactcaggactgatagggcccctgctggtgtgccacaccaacaccctgaacccagcccatggcagacaggtgactgtgcaggagtttgctctgttcttcaccatctttgatgagaccaagagctggtacttcacagagaacatggagagaaactgcagagccccctgcaacatccagatggaggaccccaccttcaaggagaactacagattccatgctatcaatggatacatcatggacaccctgccaggcctggtgatggcccaggaccagagaatcagatggtacctgctgagcatgggcagcaatgagaacatccacagcatccacttcagtggtcatgtgttcacagtaagaaagaaggaggagtacaagatggccctgtacaacctgtacccaggggtgtttgaaactgtggaaatgctgcccagcaaggcagggatctggagagtggagtgcctgattggagaacacctgcatgcaggcatgagcaccctgttcctggtgtacagcaacaagtgccagacccccctgggcatggccagtgggcacatcagagacttccagatcacagcctctgggcagtatggccagtgggcccccaagctggccagactgcactactcaggcagcatcaatgcctggagcaccaaggagcccttcagctggatcaaggtggacctgctggcccccatgatcatccatggcatcaagacccagggagcaagacagaagttcagcagcctgtacatcagccagttcatcatcatgtacagcctggatgggaagaagtggcagacctacagaggcaacagcactgggaccctgatggtgttctttggcaatgtagacagctctggcatcaagcacaacatcttcaacccccccatcattgcaagatacatcagactgcaccccacccactacagcatcagaagcaccctgagaatggagctgatgggctgtgacctgaacagctgcagcatgcccctgggcatggagagcaaggccatcagtgatgcccagatcactgctagcagctacttcaccaacatgtttgccacctggagccccagcaaggccagactgcacctgcagggcagaagcaatgcatggagaccccaggtgaacaaccccaaggagtggctgcaggtggacttccagaagaccatgaaggtgacaggggttaccacccagggtgtgaagagcctgctgaccagcatgtatgtgaaggagttcctgatcagcagcagccaggatggccaccagtggaccctgttcttccagaatgggaaggtgaaggtgttccagggcaaccaggacagcttcacccctgtggtgaacagcctggacccccccctgctgaccagatacctgagaatccacccccagagctgggtgcaccagattgccctgagaatggaggtgctgggctgtgaggcccaggacctgtactgatttaaatctcgagccgggcggagtgtgttagtctctccagagggaggctggttccccagggaagcagagcctgtgtgcgggcagcagctgtgtgcgggcctgggggttgttaagtgcaattatttttaataaaaggggcatttggaaaaaaaaaaaaaaggtagcagtcgacagatgaattctgcagatctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaaaaaagagaaatgttctggcacctgcacttgcactggggacagcctattttgctagtttgttttgtttcgttttgttttgatggagagcgtatgttgtttaaacgcggccgcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 35 - DNA, 6314 bp, synthetic construct
  source 1..6314: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacggcgcgccacgcgttctaggagttaatttttaaaaagcagtcaaaagtccaagtgcccttgcgagcatttactctctctgtttgctctggttaataatctcaggagcacaaacattccttactagttctaggagttaatttttaaaaagcagtcaaaagtccaagtgcccttgcgagcatttactctctctgtttgctctggttaataatctcaggagcacaaacattccttactagttctagagcggccgccagtgtgctggaattcggcttttttagggctggaagctacctttgacatcatttcctctgcgaatgcatgtataatttctacagaacctattagaaaggatcacccagcctctgcttttgtacaactttcccttaaaaaactgccaatcccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactgagagtgctctagttctgcaatacaggacatgctataaaaatggaaagatgttgctttctgagagatcaggtggattcttgggcatttgcttaagcttggtaccgccaccatgcagatagaactgagcacctgcttcttcctgtgcctgctgagattctgcttctctgccaccagaagatactacctgggggctgtggagctgagctgggactacatgcagagtgacctgggagagctgccagttgatgcaagattcccccccagagtgcccaagagcttccccttcaacacctctgtggtgtacaagaagaccctgtttgtggagttcactgatcacctgttcaacatagccaagcccagacccccctggatgggcctgctgggccccaccatccaggctgaagtgtatgacactgtagtgatcaccctgaagaacatggccagccaccctgtgagcctgcatgctgtgggagtgagctactggaaggcctcagagggagcagagtatgatgaccagaccagccagagagagaaggaggatgacaaggtgttcccaggaggcagccacacctatgtttggcaggtgctgaaggagaatgggcccatggcctcagaccccctgtgcctgacctacagctacctgagccatgtagacctggtgaaggacctgaactctggcctgataggagccctgctggtgtgcagagagggcagcctggccaaggagaagacccagaccctgcacaagttcatcctgctgtttgcagtgtttgatgagggcaagagctggcactcagagaccaagaacagcctgatgcaggacagagatgcagccagtgctagagcctggcccaagatgcacacagtgaatgggtatgttaacagaagcctgccaggcctgataggatgccacagaaagtctgtgtactggcatgtgattggtatgggcaccaccccagaggtgcacagcatcttcctggagggccacaccttcctggtgagaaaccacagacaggccagcctggagatcagccccatcaccttcctgacagcccagaccctgctgatggacctgggccagttcctgctgttctgccacatcagcagccaccagcatgatggcatggaggcctatgttaaggtggacagctgcccagaggagccccagctgcagatgaagaacaatgaagaggcagaggactatgatgatgacctgacagattcagagatggatgtggtgagatttgatgatgacaacagccccagcttcatccagatcagaagtgtggccaagaagcaccccaagacctgggtgcactacattgctgctgaagaggaggactgggactatgcccccctggtgctggccccagatgacagaagctacaagagccagtacctgaacaatgggccccagagaattggaagaaagtacaagaaggtgagattcatggcctacacagatgaaaccttcaagaccagagaggccatccagcatgagagtgggatcctgggccccctgctgtatggggaggtgggtgataccctgctgatcatcttcaagaaccaggccagcagaccctacaacatctacccccatggcatcacagatgtgagacccctgtacagcagaagactgcccaagggagttaagcacctgaaggacttccccatcctgccaggagagatcttcaagtacaagtggacagtcacagtggaggatgggcccaccaagtctgaccccagatgcctgaccagatactacagcagctttgtaaacatggagagagacctggcctcaggtctgataggccccctgctgatctgctacaaggagagtgttgatcagcagggcaaccagatcatgtctgacaagagaaatgtgatcctgttctcagtgtttgatgaaaacagaagctggtacctgactgagaacatccagagattcctgcccaacccagctggggtgcagctggaggaccctgagttccaggccagcaacatcatgcacagcatcaatggatatgtgtttgacagcctgcagctgtcagtgtgcctgcatgaggtggcctactggtacatcctgagcataggagctcagacagacttcctgagtgtattcttctcagggtacaccttcaagcacaagatggtgtatgaggacaccctgaccctgttccccttctcaggtgagacagtgttcatgagcatggagaacccagggctgtggatcctgggctgccacaactctgatttcagaaacagaggcatgactgccctgctgaaggtgagcagctgtgacaagaacacaggggactactatgaggacagctatgaggacatctctgcctacctgctgagcaagaacaatgctattgagcccagaagcttcagccagaacccccctgtactgaagagacaccagagagagatcaccagaaccaccctgcagagtgatcaggaggagatagattatgatgacaccatctcagtggagatgaagaaggaggactttgatatctatgatgaggatgaaaaccagagccccagaagcttccagaagaagaccagacactacttcattgcagctgtagagagactgtgggactatggtatgagcagcagcccccatgtcctgagaaacagagcccagtctggatcagtcccccagttcaagaaggtggtgttccaggagttcacagatggtagcttcacccagcccctgtacagaggagagctgaatgagcacctgggcctgctgggcccctacatcagagcagaggtggaggacaacatcatggtgaccttcagaaaccaggccagcagaccctacagcttctacagcagcctgatcagctatgaagaggaccagagacagggagcagagcccagaaagaactttgtgaagcccaatgagaccaagacctacttctggaaggtgcagcaccacatggcccccaccaaggatgagtttgattgcaaggcctgggcctacttcagtgatgttgacctggagaaggatgttcacagtggcctgataggtcccctgctggtgtgccacaccaacaccctgaacccagcccatggcagacaggtgactgtccaggagtttgccctgttcttcaccatctttgatgagaccaagagctggtacttcactgaaaacatggagagaaactgcagagccccctgcaacatccagatggaggaccccaccttcaaggagaactacagattccatgccatcaatggctacatcatggacaccctgcctggactggtgatggcccaggaccagagaatcagatggtacctgctgagcatgggcagcaatgagaacatccacagcatccacttctcaggccatgtattcactgtgagaaagaaggaggagtacaagatggccctgtacaacctgtacccaggtgtgtttgagacagtggagatgctgcccagcaaggcaggtatctggagagtggagtgcctgataggagaacacctgcatgctggcatgagcaccctgttcctggtgtacagcaacaagtgccagacccccctgggcatggccagtggccacatcagagacttccagatcacagcctcaggccagtatgggcagtgggcccccaagctggccagactgcactactctggcagcatcaatgcatggagcaccaaggagcccttcagctggatcaaggtggacctgctggcccccatgatcatccatggaatcaagacccagggtgctagacagaagttcagcagcctgtacatcagccagttcatcatcatgtacagcctggatggcaagaagtggcagacctacagaggcaacagcactggcaccctgatggtgttctttggcaatgttgacagctctggcatcaagcacaacatcttcaacccccccatcatagcaagatacatcagactgcaccccacccactacagcatcagaagcaccctgagaatggagctgatgggctgtgatctgaacagctgcagcatgcccctgggcatggagagcaaggccatcagtgatgctcagatcactgccagcagctacttcaccaacatgtttgcaacctggagccccagcaaggccagactgcacctgcagggcagaagcaatgcctggagaccccaggtgaacaaccccaaggagtggctgcaggtggacttccagaagaccatgaaggtgacaggtgttaccacccagggtgtcaagagcctgctgaccagcatgtatgtaaaggagttcctgatcagcagcagccaggatggccaccagtggaccctgttcttccagaatggaaaggtgaaggtgttccagggcaaccaggacagcttcacccctgtggtgaacagcctggacccccccctgctgaccagatacctgagaatccacccccagagctgggtgcaccagatagccctgagaatggaggtgctgggctgtgaagcccaggacctgtactgatttaaatctcgagccgggcggagtgtgttagtctctccagagggaggctggttccccagggaagcagagcctgtgtgcgggcagcagctgtgtgcgggcctgggggttgttaagtgcaattatttttaataaaaggggcatttggaaaaaaaaaaaaaaggtagcagtcgacagatgaattctgcagatctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaaaaaagagaaatgttctggcacctgcacttgcactggggacagcctattttgctagtttgttttgtttcgttttgttttgatggagagcgtatgttgtttaaacgcggccgcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 36 - DNA, 6314 bp, synthetic construct
  source 1..6314: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacggcgcgccacgcgttctaggagttaatttttaaaaagcagtcaaaagtccaagtgcccttgcgagcatttactctctctgtttgctctggttaataatctcaggagcacaaacattccttactagttctaggagttaatttttaaaaagcagtcaaaagtccaagtgcccttgcgagcatttactctctctgtttgctctggttaataatctcaggagcacaaacattccttactagttctagagcggccgccagtgtgctggaattcggcttttttagggctggaagctacctttgacatcatttcctctgcgaatgcatgtataatttctacagaacctattagaaaggatcacccagcctctgcttttgtacaactttcccttaaaaaactgccaatcccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactgagagtgctctagttctgcaatacaggacatgctataaaaatggaaagatgttgctttctgagagatcaggtggattcttgggcatttgcttaagcttggtaccgccaccatgcagatagagctgagcacctgcttcttcctgtgcctgctgagattctgcttctctgccaccagaagatactacctgggtgctgttgagctgagctgggactacatgcagtctgatctgggtgagctgcctgttgatgccagattcccccccagagtgcccaagagcttccccttcaacacctcagttgtgtacaagaagaccctgtttgttgagttcacagaccacctgttcaacattgctaagcccagacccccctggatgggcctgctgggccccaccatccaggcagaggtgtatgatacagttgtgatcaccctgaagaacatggccagccacccagtgagcctgcatgcagtaggtgtgagctactggaaggccagtgagggggctgaatatgatgatcagaccagccagagagagaaggaggatgacaaggtgttcccaggtggtagccacacctatgtatggcaggtgctgaaggagaatggccccatggccagtgaccccctgtgcctgacctacagctacctgagccatgtggacctggtgaaggacctgaactctggactgattggagcactgctggtgtgcagagagggcagcctggccaaggagaagacccagaccctgcacaagttcatcctgctgtttgcagtgtttgatgagggcaagagctggcactcagagaccaagaacagcctgatgcaggacagagatgcagcttctgccagagcctggcccaagatgcacactgtcaatggctatgtgaacagaagcctgccaggcctgattggttgccacagaaagagtgtgtactggcatgttatagggatgggcaccacccctgaggtgcacagcatcttcctggagggccacaccttcctggtgagaaaccacagacaggccagcctggagatcagccccatcaccttcctgacagcacagaccctgctgatggacctgggccagttcctgctgttctgccacatcagcagccaccagcatgatggcatggaggcctatgttaaggtggacagctgccctgaggagccccagctgcagatgaagaacaatgaggaggctgaggactatgatgatgacctgacagactcagagatggatgtggtgagatttgatgatgataacagccccagcttcatccagatcagatcagtggccaagaagcaccccaagacctgggtgcactacatagctgctgaagaggaggactgggactatgcccccctggtgctggccccagatgacagaagctacaagagccagtacctgaacaatgggccccagagaattggcagaaagtacaagaaggtgagattcatggcctacactgatgagaccttcaagaccagagaggccatccagcatgagtctggaatcctgggccccctgctgtatggagaggtgggtgacaccctgctgatcatcttcaagaaccaggccagcagaccctacaacatctacccccatggcatcactgatgttagacccctgtacagcagaagactgcccaagggtgtgaagcacctgaaggacttccccatcctgcctggggagatcttcaagtacaagtggacagtcactgtagaggatggccccaccaagagtgaccccagatgcctgaccagatactacagcagctttgtgaacatggagagagacctggcctctggactgattggacccctgctgatctgctacaaggagagtgtggaccagcagggcaaccagatcatgtcagataagagaaatgtcatcctgttctcagtatttgatgagaacagaagctggtacctgactgagaacatccagagattcctgcccaacccagctggggtgcagctggaggaccctgaattccaggccagcaacatcatgcacagcatcaatggctatgtatttgatagcctgcagctgtctgtgtgcctgcatgaggtggcctactggtacatcctgagcattggggcccagactgatttcctgtctgtcttcttctctgggtacaccttcaagcacaagatggtgtatgaggacaccctgaccctgttccccttctcaggagagacagtgttcatgagcatggagaacccaggcctgtggatcctgggctgccacaactcagacttcagaaacagaggcatgactgccctgctgaaggtgagcagctgtgacaagaacacaggtgactactatgaggacagctatgaagacatctctgcctacctgctgagcaagaacaatgctatagaacccagaagcttcagccagaacccccctgtgctgaagagacaccagagagagatcaccagaaccaccctgcagtcagatcaggaggagatagattatgatgataccatctcagtagaaatgaagaaggaggactttgacatctatgatgaagatgaaaaccagagccccagaagcttccagaagaagaccagacactacttcatagctgctgtagagagactgtgggactatggcatgagcagcagcccccatgtactgagaaacagagcccagagtggctcagtgccccagttcaagaaggtggtgttccaggagttcactgatggcagcttcacccagcccctgtacagaggtgaactgaatgagcacctgggcctgctgggcccctacatcagagcagaggtggaggacaacatcatggtgaccttcagaaaccaggccagcagaccctacagcttctacagcagcctgatcagctatgaagaggaccagagacagggtgctgagcccagaaagaactttgtgaagcccaatgagaccaagacctacttctggaaggtgcagcaccacatggcccccaccaaggatgagtttgactgcaaggcctgggcctacttcagtgatgtagacctggagaaggatgtgcacagtgggctgataggtcccctgctggtgtgccacaccaacaccctgaacccagcacatggtagacaggtgacagttcaggagtttgccctgttcttcaccatctttgatgaaaccaagagctggtacttcactgagaacatggagagaaactgcagagccccctgcaacatccagatggaggaccccaccttcaaggagaactacagattccatgccatcaatggctacatcatggacaccctgccaggcctggtgatggcccaggaccagagaatcagatggtacctgctgagcatgggcagcaatgagaacatccacagcatccacttctcaggacatgtgttcactgtgagaaagaaggaggagtacaagatggccctgtacaacctgtacccaggggtatttgaaacagtagagatgctgcccagcaaggcaggcatctggagagtggagtgcctgattggagaacacctgcatgcaggtatgagcaccctgttcctggtgtacagcaacaagtgccagacccccctgggcatggcctcaggccacatcagagacttccagatcacagcttctggccagtatgggcagtgggcccccaagctggccagactgcactactcaggtagcatcaatgcttggagcaccaaggagcccttcagctggatcaaggtggacctgctggcccccatgatcatccatgggatcaagacccagggagccagacagaagttcagcagcctgtacatcagccagttcatcatcatgtacagcctggatggtaagaagtggcagacctacagaggcaacagcactggcaccctgatggtgttctttggaaatgttgatagctcagggatcaagcacaacatcttcaacccccccatcatagccagatacatcagactgcaccccacccactacagcatcagaagcaccctgagaatggagctgatgggctgtgatctgaacagctgcagcatgcccctgggcatggagagcaaggccatctctgatgcacagatcacagcaagcagctacttcaccaacatgtttgccacctggagccccagcaaggccagactgcacctgcagggcagaagcaatgcttggagaccccaggtgaacaaccccaaggagtggctgcaggtggacttccagaagaccatgaaggtgacaggggtgaccacccagggggtgaagagcctgctgaccagcatgtatgtcaaggagttcctgatcagcagcagccaggatggccaccagtggaccctgttcttccagaatgggaaggtgaaggtgttccagggcaaccaggacagcttcaccccagtagtgaacagcctggacccccccctgctgaccagatacctgagaatccacccccagagctgggtgcaccagattgctctgagaatggaggtgctgggctgtgaggcccaggacctgtactgatttaaatctcgagccgggcggagtgtgttagtctctccagagggaggctggttccccagggaagcagagcctgtgtgcgggcagcagctgtgtgcgggcctgggggttgttaagtgcaattatttttaataaaaggggcatttggaaaaaaaaaaaaaaggtagcagtcgacagatgaattctgcagatctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaaaaaagagaaatgttctggcacctgcacttgcactggggacagcctattttgctagtttgttttgtttcgttttgttttgatggagagcgtatgttgtttaaacgcggccgcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 37 - DNA, 6314 bp, synthetic construct
  source 1..6314: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacggcgcgccacgcgttctaggagttaatttttaaaaagcagtcaaaagtccaagtgcccttgcgagcatttactctctctgtttgctctggttaataatctcaggagcacaaacattccttactagttctaggagttaatttttaaaaagcagtcaaaagtccaagtgcccttgcgagcatttactctctctgtttgctctggttaataatctcaggagcacaaacattccttactagttctagagcggccgccagtgtgctggaattcggcttttttagggctggaagctacctttgacatcatttcctctgcgaatgcatgtataatttctacagaacctattagaaaggatcacccagcctctgcttttgtacaactttcccttaaaaaactgccaatcccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactgagagtgctctagttctgcaatacaggacatgctataaaaatggaaagatgttgctttctgagagatcaggtggattcttgggcatttgcttaagcttggtaccgccaccatgcagatagaactgagcacctgcttcttcctgtgcctgctgagattctgcttctctgccaccagaagatactacctgggggctgtggagctgagctgggactacatgcagtctgacctgggagaactgcctgttgatgctagattcccccccagagtgcccaagagcttccccttcaacacctcagtggtgtacaagaagaccctgtttgtagagttcactgaccacctgttcaacattgcaaagcccagacccccctggatgggcctgctgggccccaccatccaggctgaggtgtatgatactgttgtgatcaccctgaagaacatggccagccaccctgtgagcctgcatgctgtgggagtcagctactggaaggcctcagagggggctgagtatgatgaccagaccagccagagagagaaggaggatgacaaggtgttccctggtgggagccacacctatgtgtggcaggtgctgaaggagaatggacccatggcctcagaccccctgtgcctgacctacagctacctgagccatgtagacctggtgaaggacctgaacagtggtctgataggtgcactgctggtgtgcagagagggcagcctggccaaggagaagacccagaccctgcacaagttcatcctgctgtttgcagtgtttgatgagggcaagagctggcactctgagaccaagaacagcctgatgcaggacagagatgctgcctcagccagagcctggcccaagatgcacactgttaatgggtatgtgaacagaagcctgccaggcctgattggctgccacagaaagagtgtgtactggcatgtcataggaatgggcaccacccctgaggtgcacagcatcttcctggagggccacaccttcctggtgagaaaccacagacaggccagcctggagatcagccccatcaccttcctgactgcacagaccctgctgatggacctgggccagttcctgctgttctgccacatcagcagccaccagcatgatgggatggaggcctatgtaaaggtggacagctgccctgaagagccccagctgcagatgaagaacaatgaggaggcagaagactatgatgatgatctgacagacagtgaaatggatgtggtgagatttgatgatgacaacagccccagcttcatccagatcagaagtgtggccaagaagcaccccaagacctgggtgcactacattgctgcagaggaggaggactgggactatgcccccctggtgctggccccagatgacagaagctacaagagccagtacctgaacaatgggccccagagaatagggagaaagtacaagaaggtgagattcatggcctacacagatgagaccttcaagaccagagaggccatccagcatgagagtggtatcctgggccccctgctgtatggagaagtgggggataccctgctgatcatcttcaagaaccaggccagcagaccctacaacatctacccccatggcatcacagatgtgagacccctgtacagcagaagactgcccaagggtgtaaagcacctgaaggacttccccatcctgccaggggaaatcttcaagtacaagtggactgtgactgtggaggatggacccaccaagagtgatcccagatgcctgaccagatactacagcagctttgttaacatggagagagacctggcctcaggactgattggccccctgctgatctgctacaaggagagtgtggaccagcagggcaaccagatcatgagtgacaagagaaatgtcatcctgttcagtgtctttgatgaaaacagaagctggtacctgacagaaaacatccagagattcctgcccaaccctgcaggtgtgcagctggaggacccagagttccaggccagcaacatcatgcacagcatcaatgggtatgtgtttgatagcctgcagctgagtgtatgcctgcatgaggtggcctactggtacatcctgagcataggtgctcagactgacttcctgtcagtattcttctcaggctacaccttcaagcacaagatggtgtatgaggacaccctgaccctgttccccttcagtggggaaacagtgttcatgagcatggagaaccctgggctgtggatcctgggctgccacaacagtgacttcagaaacagaggcatgacagccctgctgaaggtgagcagctgtgacaagaacacaggagactactatgaagacagctatgaagacatcagtgcttacctgctgagcaagaacaatgcaatagagcccagaagcttcagccagaaccccccagtgctgaagagacaccagagagagatcaccagaaccaccctgcagtcagaccaggaggagattgactatgatgacaccatctctgtggagatgaagaaggaggactttgacatctatgatgaggatgagaaccagagccccagaagcttccagaagaagaccagacactacttcatagcagcagtggagagactgtgggactatgggatgagcagcagcccccatgtcctgagaaacagagcccagtcaggaagtgtcccccagttcaagaaggtggtgttccaggagttcactgatgggagcttcacccagcccctgtacagaggagagctgaatgaacacctgggcctgctgggcccctacatcagagcagaggtggaggacaacatcatggtgaccttcagaaaccaggccagcagaccctacagcttctacagcagcctgatcagctatgaggaggaccagagacagggagctgaacccagaaagaactttgtgaagcccaatgaaaccaagacctacttctggaaggtgcagcaccacatggcccccaccaaggatgagtttgactgcaaggcctgggcctacttctctgatgttgatctggagaaggatgtacactctgggctgattggccccctgctggtgtgccacaccaacaccctgaacccagcccatggcagacaggtgactgtgcaggagtttgccctgttcttcaccatctttgatgagaccaagagctggtacttcactgaaaacatggagagaaactgcagagccccctgcaacatccagatggaggaccccaccttcaaggagaactacagattccatgctatcaatggctacatcatggacaccctgcctggactggtgatggcccaggaccagagaatcagatggtacctgctgagcatgggcagcaatgaaaacatccacagcatccacttctcaggccatgtattcactgtgagaaagaaggaggagtacaagatggccctgtacaacctgtacccaggtgtgtttgaaacagtggaaatgctgcccagcaaggctggcatctggagagtggagtgcctgattggtgagcacctgcatgctggaatgagcaccctgttcctggtgtacagcaacaagtgccagacccccctgggcatggcctcagggcacatcagagacttccagatcactgcctctggccagtatggacagtgggcccccaagctggccagactgcactacagtggcagcatcaatgcctggagcaccaaggagcccttcagctggatcaaggtggacctgctggcccccatgatcatccatggcatcaagacccagggtgccagacagaagttcagcagcctgtacatcagccagttcatcatcatgtacagcctggatggcaagaagtggcagacctacagaggcaacagcacaggtaccctgatggtgttctttggcaatgtggacagctctggtatcaagcacaacatcttcaacccccccatcatagccagatacatcagactgcaccccacccactacagcatcagaagcaccctgagaatggagctgatgggctgtgatctgaacagctgcagcatgcccctgggcatggagagcaaggccatctctgatgctcagatcacagctagcagctacttcaccaacatgtttgccacctggagccccagcaaggccagactgcacctgcagggcagaagcaatgcatggagaccccaggtgaacaaccccaaggagtggctgcaggtggacttccagaagaccatgaaggtgacaggagtgaccacccagggagtgaagagcctgctgaccagcatgtatgtgaaggagttcctgatcagcagcagccaggatggccaccagtggaccctgttcttccagaatgggaaggtgaaggtgttccagggcaaccaggacagcttcaccccagtagtgaacagcctggacccccccctgctgaccagatacctgagaatccacccccagagctgggtgcaccagatagctctgagaatggaggtgctgggctgtgaagcccaggacctgtactgatttaaatctcgagccgggcggagtgtgttagtctctccagagggaggctggttccccagggaagcagagcctgtgtgcgggcagcagctgtgtgcgggcctgggggttgttaagtgcaattatttttaataaaaggggcatttggaaaaaaaaaaaaaaggtagcagtcgacagatgaattctgcagatctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaaaaaagagaaatgttctggcacctgcacttgcactggggacagcctattttgctagtttgttttgtttcgttttgttttgatggagagcgtatgttgtttaaacgcggccgcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 38 - DNA, 6314 bp, synthetic construct
  source 1..6314: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacggcgcgccacgcgttctaggagttaatttttaaaaagcagtcaaaagtccaagtgcccttgcgagcatttactctctctgtttgctctggttaataatctcaggagcacaaacattccttactagttctaggagttaatttttaaaaagcagtcaaaagtccaagtgcccttgcgagcatttactctctctgtttgctctggttaataatctcaggagcacaaacattccttactagttctagagcggccgccagtgtgctggaattcggcttttttagggctggaagctacctttgacatcatttcctctgcgaatgcatgtataatttctacagaacctattagaaaggatcacccagcctctgcttttgtacaactttcccttaaaaaactgccaatcccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactgagagtgctctagttctgcaatacaggacatgctataaaaatggaaagatgttgctttctgagagatcaggtggattcttgggcatttgcttaagcttggtaccgccaccatgcagatagaactgagcacctgcttcttcctgtgcctgctgagattctgcttcagtgccaccagaagatactacctgggtgctgttgagctgagctgggactacatgcagtctgacctgggtgaactgccagtagatgcaagattcccccccagagtgcccaagagcttccccttcaacacctctgtagtctacaagaagaccctgtttgtggagttcacagatcacctgttcaacattgctaagcccagacccccctggatgggcctgctgggccccaccatccaggcagaggtgtatgacactgtagtgatcaccctgaagaacatggccagccacccagtgagcctgcatgctgtgggtgtgagctactggaaggccagtgaaggagcagagtatgatgatcagaccagccagagagagaaggaggatgacaaggtgttcccaggtggaagccacacctatgtctggcaggtgctgaaggagaatgggcccatggcctcagatcccctgtgcctgacctacagctacctgagccatgttgacctggtgaaggacctgaacagtggactgataggtgctctgctggtgtgcagagagggcagcctggccaaggagaagacccagaccctgcacaagttcatcctgctgtttgcagtgtttgatgaaggcaagagctggcacagtgagaccaagaacagcctgatgcaggacagagatgcagcatcagccagagcctggcccaagatgcacacagtgaatggctatgtgaacagaagcctgccaggcctgataggatgccacagaaagtctgtatactggcatgtcattggtatgggcaccacccctgaagtgcacagcatcttcctggagggccacaccttcctggtgagaaaccacagacaggccagcctggagatcagccccatcaccttcctgacagcccagaccctgctgatggacctgggccagttcctgctgttctgccacatcagcagccaccagcatgatgggatggaggcctatgtcaaggtggacagctgcccagaggagccccagctgcagatgaagaacaatgaggaggctgaggactatgatgatgacctgactgactcagagatggatgtggtgagatttgatgatgataacagccccagcttcatccagatcagatctgtggccaagaagcaccccaagacctgggtgcactacatagctgcagaagaggaggactgggactatgcccccctggtgctggccccagatgatagaagctacaagagccagtacctgaacaatggcccccagagaattgggagaaagtacaagaaggtgagattcatggcctacacagatgaaaccttcaagaccagagaggccatccagcatgagtctggcatcctgggccccctgctgtatggggaagtgggagacaccctgctgatcatcttcaagaaccaggccagcagaccctacaacatctacccccatggtatcacagatgtgagacccctgtacagcagaagactgcccaagggggtgaagcacctgaaggacttccccatcctgcctggggagatcttcaagtacaagtggacagtaactgtggaggatggccccaccaagtctgaccccagatgcctgaccagatactacagcagctttgtgaacatggagagagacctggccagtggcctgattggccccctgctgatctgctacaaggagagtgttgaccagcagggcaaccagatcatgagtgataagagaaatgttatcctgttcagtgtgtttgatgaaaacagaagctggtacctgacagagaacatccagagattcctgcccaacccagctggtgtccagctggaggaccctgagttccaggccagcaacatcatgcacagcatcaatggctatgtatttgacagcctgcagctgagtgtgtgcctgcatgaagtggcctactggtacatcctgagcataggtgctcagacagacttcctgagtgtcttcttcagtggctacaccttcaagcacaagatggtgtatgaagacaccctgaccctgttccccttcagtggagagacagtgttcatgagcatggagaacccaggactgtggatcctgggctgccacaacagtgacttcagaaacagaggcatgacagccctgctgaaggtgagcagctgtgacaagaacactggggactactatgaggacagctatgaggacatctctgcctacctgctgagcaagaacaatgcaatagagcccagaagcttcagccagaacccccctgtgctgaagagacaccagagagagatcaccagaaccaccctgcagtcagatcaggaggagatagactatgatgataccatctctgtggagatgaagaaggaggactttgatatctatgatgaggatgagaaccagagccccagaagcttccagaagaagaccagacactacttcatagctgcagtggagagactgtgggactatggcatgagcagcagcccccatgtactgagaaacagagcccagtcaggatctgtcccccagttcaagaaggtggtgttccaggagttcactgatgggagcttcacccagcccctgtacagaggtgaactgaatgaacacctgggcctgctgggcccctacatcagagcagaggtggaggacaacatcatggtgaccttcagaaaccaggccagcagaccctacagcttctacagcagcctgatcagctatgaagaggaccagagacagggagctgagcccagaaagaactttgtgaagcccaatgaaaccaagacctacttctggaaggtgcagcaccacatggcccccaccaaggatgaatttgactgcaaggcctgggcctacttctcagatgtagacctggagaaggatgtgcactcaggactgattggccccctgctggtgtgccacaccaacaccctgaacccagcacatggcagacaggtgacagttcaggagtttgcactgttcttcaccatctttgatgagaccaagagctggtacttcacagagaacatggagagaaactgcagagccccctgcaacatccagatggaggaccccaccttcaaggagaactacagattccatgctatcaatggctacatcatggacaccctgccagggctggtgatggcccaggaccagagaatcagatggtacctgctgagcatgggcagcaatgaaaacatccacagcatccacttcagtggccatgtgttcacagttagaaagaaggaggagtacaagatggccctgtacaacctgtaccctggtgtgtttgaaacagtggagatgctgcccagcaaggctggcatctggagagtggagtgcctgattggggaacacctgcatgcaggaatgagcaccctgttcctggtgtacagcaacaagtgccagacccccctgggcatggcctcaggccacatcagagacttccagatcactgccagtgggcagtatggtcagtgggcccccaagctggccagactgcactactctggcagcatcaatgcctggagcaccaaggagcccttcagctggatcaaggtggacctgctggcccccatgatcatccatggcatcaagacccagggggccagacagaagttcagcagcctgtacatcagccagttcatcatcatgtacagcctggatggcaagaagtggcagacctacagaggcaacagcactggaaccctgatggtgttctttggcaatgtagacagctcaggcatcaagcacaacatcttcaacccccccatcattgccagatacatcagactgcaccccacccactacagcatcagaagcaccctgagaatggagctgatgggctgtgacctgaacagctgcagcatgcccctgggcatggagagcaaggccatctctgatgcccagatcactgccagcagctacttcaccaacatgtttgccacctggagccccagcaaggccagactgcacctgcagggcagaagcaatgcttggagaccccaggtgaacaaccccaaggagtggctgcaggtggacttccagaagaccatgaaggtgactggtgtgaccacccagggtgtgaagagcctgctgaccagcatgtatgtcaaggagttcctgatcagcagcagccaggatggtcaccagtggaccctgttcttccagaatggcaaggtgaaggtgttccagggcaaccaggacagcttcaccccagtggtgaacagcctggacccccccctgctgaccagatacctgagaatccacccccagagctgggtgcaccagatagccctgagaatggaggtgctgggctgtgaggcccaggacctgtactgatttaaatctcgagccgggcggagtgtgttagtctctccagagggaggctggttccccagggaagcagagcctgtgtgcgggcagcagctgtgtgcgggcctgggggttgttaagtgcaattatttttaataaaaggggcatttggaaaaaaaaaaaaaaggtagcagtcgacagatgaattctgcagatctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaaaaaagagaaatgttctggcacctgcacttgcactggggacagcctattttgctagtttgttttgtttcgttttgttttgatggagagcgtatgttgtttaaacgcggccgcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 39 - DNA, 6314 bp, synthetic construct
  source 1..6314: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacggcgcgccacgcgttctaggagttaatttttaaaaagcagtcaaaagtccaagtgcccttgcgagcatttactctctctgtttgctctggttaataatctcaggagcacaaacattccttactagttctaggagttaatttttaaaaagcagtcaaaagtccaagtgcccttgcgagcatttactctctctgtttgctctggttaataatctcaggagcacaaacattccttactagttctagagcggccgccagtgtgctggaattcggcttttttagggctggaagctacctttgacatcatttcctctgcgaatgcatgtataatttctacagaacctattagaaaggatcacccagcctctgcttttgtacaactttcccttaaaaaactgccaatcccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactgagagtgctctagttctgcaatacaggacatgctataaaaatggaaagatgttgctttctgagagatcaggtggattcttgggcatttgcttaagcttggtaccgccaccatgcagattgaactgagcacctgcttcttcctgtgcctgctgagattctgcttcagtgccaccagaagatactacctgggggcagtggagctgagctgggactacatgcagagtgacctgggggaactgcctgttgatgccagattcccccccagagtgcccaagagcttccccttcaacacctcagttgtgtacaagaagaccctgtttgtggagttcactgaccacctgttcaacattgctaagcccagacccccctggatgggcctgctgggccccaccatccaggctgaggtgtatgacactgtggtgatcaccctgaagaacatggccagccaccctgtgagcctgcatgcagtgggggtgagctactggaaggcctcagaaggggcagagtatgatgatcagaccagccagagagagaaggaggatgacaaggtgttccctggtggtagccacacctatgtgtggcaggtgctgaaggagaatggccccatggcctcagaccccctgtgcctgacctacagctacctgagccatgtggacctggtgaaggacctgaacagtggcctgattggagctctgctggtgtgcagagagggcagcctggccaaggagaagacccagaccctgcacaagttcatcctgctgtttgctgtgtttgatgagggcaagagctggcactcagagaccaagaacagcctgatgcaggacagagatgctgccagtgcaagagcctggcccaagatgcacactgtcaatggatatgtgaacagaagcctgccaggactgataggctgccacagaaagtctgtatactggcatgttataggtatgggcaccaccccagaggtgcacagcatcttcctggagggccacaccttcctggtgagaaaccacagacaggccagcctggagatcagccccatcaccttcctgactgcccagaccctgctgatggacctgggccagttcctgctgttctgccacatcagcagccaccagcatgatggtatggaggcctatgtgaaggtggacagctgccctgaggagccccagctgcagatgaagaacaatgaggaggcagaggactatgatgatgacctgacagacagtgagatggatgtggtgagatttgatgatgacaacagccccagcttcatccagatcagaagtgtggccaagaagcaccccaagacctgggtgcactacattgctgctgaagaggaggactgggactatgcacccctggtgctggccccagatgatagaagctacaagagccagtacctgaacaatggaccccagagaattggaagaaagtacaagaaggtgagattcatggcctacactgatgagaccttcaagaccagagaggccatccagcatgaatcagggatcctgggccccctgctgtatggagaggtgggagacaccctgctgatcatcttcaagaaccaggccagcagaccctacaacatctacccccatggtatcactgatgtaagacccctgtacagcagaagactgcccaagggagtgaagcacctgaaggacttccccatcctgcctggggaaatcttcaagtacaagtggacagtcacagttgaagatggacccaccaagagtgatcccagatgcctgaccagatactacagcagctttgtgaacatggagagagacctggcctcaggcctgataggccccctgctgatctgctacaaggagtctgttgaccagcagggcaaccagatcatgagtgacaagagaaatgtgatcctgttctcagtgtttgatgagaacagaagctggtacctgactgaaaacatccagagattcctgcccaacccagcaggggtgcagctggaggacccagagttccaggccagcaacatcatgcacagcatcaatggctatgtctttgatagcctgcagctgtctgtatgcctgcatgaggtggcctactggtacatcctgagcataggagcccagactgatttcctgagtgttttcttcagtggctacaccttcaagcacaagatggtgtatgaggacaccctgaccctgttccccttctctggtgagactgtgttcatgagcatggagaaccctggtctgtggatcctgggctgccacaactcagatttcagaaacagaggcatgacagccctgctgaaggtgagcagctgtgacaagaacactggggactactatgaagacagctatgaagacatctctgcttacctgctgagcaagaacaatgccattgagcccagaagcttcagccagaacccccctgtgctgaagagacaccagagagagatcaccagaaccaccctgcagtcagaccaggaggagatagactatgatgataccatctctgtggagatgaagaaggaggactttgatatctatgatgaggatgaaaaccagagccccagaagcttccagaagaagaccagacactacttcatagcagctgtagagagactgtgggactatggcatgagcagcagcccccatgtactgagaaacagagcccagagtggaagtgtgccccagttcaagaaggtggtgttccaggagttcacagatggcagcttcacccagcccctgtacagaggagaactgaatgagcacctgggcctgctgggcccctacatcagagctgaggtggaggacaacatcatggtgaccttcagaaaccaggccagcagaccctacagcttctacagcagcctgatcagctatgaggaggaccagagacagggagctgagcccagaaagaactttgtcaagcccaatgagaccaagacctacttctggaaggtgcagcaccacatggcccccaccaaggatgaatttgactgcaaggcctgggcctacttctcagatgtggacctggagaaggatgtgcactcaggtctgattggccccctgctggtgtgccacaccaacaccctgaacccagcccatggtagacaggtgactgtgcaggagtttgctctgttcttcaccatctttgatgagaccaagagctggtacttcacagagaacatggagagaaactgcagagccccctgcaacatccagatggaggaccccaccttcaaggagaactacagattccatgccatcaatgggtacatcatggacaccctgccaggtctggtgatggcccaggaccagagaatcagatggtacctgctgagcatgggcagcaatgaaaacatccacagcatccacttcagtggacatgtgttcacagttagaaagaaggaggagtacaagatggccctgtacaacctgtaccctggagtgtttgaaactgtggagatgctgcccagcaaggcaggcatctggagagtggagtgcctgataggggagcacctgcatgctggtatgagcaccctgttcctggtgtacagcaacaagtgccagacccccctgggcatggcctcaggccacatcagagacttccagatcactgctagtggccagtatggtcagtgggcccccaagctggccagactgcactactctggaagcatcaatgcctggagcaccaaggagcccttcagctggatcaaggtggacctgctggcccccatgatcatccatgggatcaagacccagggggccagacagaagttcagcagcctgtacatcagccagttcatcatcatgtacagcctggatggaaagaagtggcagacctacagaggcaacagcacaggcaccctgatggtgttctttggcaatgtagacagctcaggcatcaagcacaacatcttcaacccccccatcatagcaagatacatcagactgcaccccacccactacagcatcagaagcaccctgagaatggagctgatgggctgtgacctgaacagctgcagcatgcccctgggcatggagagcaaggccatctcagatgcccagatcacagccagcagctacttcaccaacatgtttgccacctggagccccagcaaggccagactgcacctgcagggcagaagcaatgcctggagaccccaggtgaacaaccccaaggagtggctgcaggtggacttccagaagaccatgaaggtgactggtgtgaccacccagggtgttaagagcctgctgaccagcatgtatgtgaaggagttcctgatcagcagcagccaggatgggcaccagtggaccctgttcttccagaatggaaaggtgaaggtgttccagggcaaccaggacagcttcacccctgttgtgaacagcctggacccccccctgctgaccagatacctgagaatccacccccagagctgggtgcaccagatagcactgagaatggaggtgctgggctgtgaggcccaggacctgtactgatttaaatctcgagccgggcggagtgtgttagtctctccagagggaggctggttccccagggaagcagagcctgtgtgcgggcagcagctgtgtgcgggcctgggggttgttaagtgcaattatttttaataaaaggggcatttggaaaaaaaaaaaaaaggtagcagtcgacagatgaattctgcagatctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaaaaaagagaaatgttctggcacctgcacttgcactggggacagcctattttgctagtttgttttgtttcgttttgttttgatggagagcgtatgttgtttaaacgcggccgcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 40 - DNA, 43 bp, synthetic construct
  source 1..43: mol_type = other DNA
caggtggattcttgggcatttgcttaagcttggtaccgccacc

SEQ ID NO: 41 - DNA, 24 bp, synthetic construct
  source 1..24: mol_type = other DNA
caggtggattcttgggcatttgct

SEQ ID NO: 42 - DNA, 195 bp, synthetic construct
  source 1..195: mol_type = other DNA
atctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttggg

SEQ ID NO: 43 - DNA, 193 bp, synthetic construct
  source 1..193: mol_type = other DNA
ctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttggg

SEQ ID NO: 44 - DNA, 193 bp, synthetic construct; identical to SEQ ID NO: 43
  source 1..193: mol_type = other DNA

SEQ ID NO: 45 - DNA, 92 bp, synthetic construct
  source 1..92: mol_type = other DNA
agagaaatgttctggcacctgcacttgcactggggacagcctattttgctagtttgttttgtttcgttttgttttgatggagagcgtatgtt

SEQ ID NO: 46 - DNA, 611 bp, synthetic construct
  source 1..611: mol_type = other DNA
gtccttcaacgtcgaactgtagcactctggatagctagctagcgtttgtagatagtttcaaggatagcaaccaacaacacgatagtttagaaattgttctagatcttctgctactactcgagagatagttgacttctagaagactctggatagagagcttcctgtggtttctatttcaacacttcgagcgatttagatagaggcgtcaattgagaacaaaaactggacacctgtttctagaacactagtgctagtcttctcaaggcttgatacgtcctgccactcacacacaaaaaaccaacacacagattaatgaaaataaagatcttttattagagagcgagtggatagtttagagtcgcttgagacttatcctgtgtccaactagccaagagtagagagagagtgactaggtggatcgagagatcgtcgtctatttgactgctagcctgtgttctcgtcgtagtccaaatcttcaatactctggcgactagtaacttgaatctgtgtatctaacacgacaaggctactcctttgagtttctagtgataagtgcgactggatctagctagttgagtttctagaaactacgtccaaagctactagcacta

SEQ ID NO: 47 - DNA, 48 bp, synthetic construct
  source 1..48: mol_type = other DNA
cacacaaaaaaccaacacacagattaatgaaaataaagatcttttatt

SEQ ID NO: 48 - DNA, 98 bp, synthetic construct
  source 1..98: mol_type = other DNA
agccgggcggagtgtgttagtctctccagagggaggctggttccccagggaagcagagcctgtgtgcgggcagcagctgtgtgcgggcctgggggttg

SEQ ID NO: 49 - DNA, 96 bp, synthetic construct
  source 1..96: mol_type = other DNA
ccgggcggagtgtgttagtctctccagagggaggctggttccccagggaagcagagcctgtgtgcgggcagcagctgtgtgcgggcctgggggttg

SEQ ID NO: 50 - DNA, 6314 bp, synthetic construct
  source 1..6314: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacggcgcgccacgcgttctaggagttaatttttaaaaagcagtcaaaagtccaagtgcccttgcgagcatttactctctctgtttgctctggttaataatctcaggagcacaaacattccttactagttctaggagttaatttttaaaaagcagtcaaaagtccaagtgcccttgcgagcatttactctctctgtttgctctggttaataatctcaggagcacaaacattccttactagttctagagcggccgccagtgtgctggaattcggcttttttagggctggaagctacctttgacatcatttcctctgcgaatgcatgtataatttctacagaacctattagaaaggatcacccagcctctgcttttgtacaactttcccttaaaaaactgccaatcccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactgagagtgctctagttctgcaatacaggacatgctataaaaatggaaagatgttgctttctgagagatcaggtggattcttgggcatttgcttaagcttggtaccgccaccatgcagattgaactgagcacctgcttcttcctgtgcctgctgagattctgcttcagtgctaccagaagatactacctgggggcagtggagctgagctgggactacatgcagtctgacctgggagagctgccagtagatgccagattcccccccagagtgcccaagagcttccccttcaacacctctgtggtgtacaagaagaccctgtttgtggagttcacagaccacctgttcaacatagctaagcccagacccccctggatgggcctgctgggccccaccatccaggcagaggtgtatgacactgttgtgatcaccctgaagaacatggccagccacccagttagcctgcatgctgttggtgttagctactggaaggcctctgagggagcagagtatgatgaccagaccagccagagagagaaggaggatgacaaggtgttcccaggggggagccacacctatgtctggcaggtgctgaaggagaatggccccatggcctcagaccccctgtgcctgacctacagctacctgagccatgtggacctggtgaaggacctgaactctggcctgattggtgcactgctggtgtgcagagagggcagcctggccaaggagaagacccagaccctgcacaagttcatcctgctgtttgctgtctttgatgagggcaagagctggcactcagaaaccaagaacagcctgatgcaggacagagatgcagcttctgctagagcctggcccaagatgcacactgtgaatggctatgtcaacagaagcctgcctggcctgataggctgccacagaaagtctgtgtactggcatgtgattgggatgggcaccaccccagaggtgcacagcatcttcctggagggccacaccttcctggtgagaaaccacagacaggccagcctggagatcagccccatcaccttcctgactgcccagaccctgctgatggacctgggccagttcctgctgttctgccacatcagcagccaccagcatgatggtatggaggcctatgtgaaggtggacagctgcccagaggagccccagctgcagatgaagaacaatgaagaggcagaggactatgatgatgacctgacagactcagaaatggatgtggtgagatttgatgatgataacagccccagcttcatccagatcagatctgttgccaagaagcaccccaagacctgggtgcactacattgcagctgaggaggaggactgggactatgctcccctggtgctggccccagatgacagaagctacaagagccagtacctgaacaatggaccccagagaatagggagaaagtacaagaaggtgagattcatggcctacacagatgagaccttcaagaccagagaggccatccagcatgaatcagggatcctgggccccctgctgtatggggaggtgggtgacaccctgctgatcatcttcaagaaccaggccagcagaccctacaacatctacccccatggaatcacagatgtcagacccctgtacagcagaagactgcccaagggagtaaagcacctgaaggacttccccatcctgccaggtgagatcttcaagtacaagtggacagtaacagtggaggatggtcccaccaagtctgaccccagatgcctgaccagatactacagcagctttgttaacatggagagagacctggccagtggcctgataggccccctgctgatctgctacaaggagtctgtagaccagcagggcaaccagatcatgtcagataagagaaatgtaatcctgttcagtgtgtttgatgagaacagaagctggtacctgacagagaacatccagagattcctgcccaaccctgcaggtgtgcagctggaggaccctgaattccaggccagcaacatcatgcacagcatcaatggatatgtttttgacagcctgcagctgtcagtttgcctgcatgaggtggcctactggtacatcctgagcattggggcccagacagatttcctgtctgtgttcttcagtgggtacaccttcaagcacaagatggtgtatgaggacaccctgaccctgttccccttctcaggtgaaactgtgttcatgagcatggagaacccagggctgtggatcctgggctgccacaactcagacttcagaaacagaggcatgactgctctgctgaaggtgagcagctgtgacaagaacacaggtgactactatgaggacagctatgaagacatctcagcttacctgctgagcaagaacaatgccatagagcccagaagcttcagccagaaccccccagttctgaagagacaccagagagagatcaccagaaccaccctgcagagtgaccaggaggagattgactatgatgacaccatctctgtagagatgaagaaggaggactttgacatctatgatgaggatgagaaccagagccccagaagcttccagaagaagaccagacactacttcatagctgcagtggagagactgtgggactatggcatgagcagcagcccccatgtgctgagaaacagagcccagagtggctcagtgccccagttcaagaaggtggtgttccaggagttcacagatggcagcttcacccagcccctgtacagaggggagctgaatgagcacctgggcctgctgggcccctacatcagagctgaggtggaggacaacatcatggtgaccttcagaaaccaggccagcagaccctacagcttctacagcagcctgatcagctatgaggaggaccagagacagggggcagagcccagaaagaactttgtaaagcccaatgaaaccaagacctacttctggaaggtgcagcaccacatggcccccaccaaggatgaatttgactgcaaggcctgggcctacttcagtgatgtggacctggagaaggatgtgcactcaggcctgattggtcccctgctggtgtgccacaccaacaccctgaacccagcccatggcagacaggtgactgtacaggagtttgccctgttcttcaccatctttgatgagaccaagagctggtacttcacagagaacatggagagaaactgcagagccccctgcaacatccagatggaggaccccaccttcaaggagaactacagattccatgctatcaatgggtacatcatggacaccctgccaggcctggtgatggcccaggaccagagaatcagatggtacctgctgagcatgggcagcaatgagaacatccacagcatccacttcagtggccatgtgttcacagtgagaaagaaggaggagtacaagatggccctgtacaacctgtaccctggagtgtttgaaactgtggagatgctgcccagcaaggcaggtatctggagagtggagtgcctgattggtgagcacctgcatgctgggatgagcaccctgttcctggtgtacagcaacaagtgccagacccccctgggcatggcctcagggcacatcagagacttccagatcactgccagtgggcagtatgggcagtgggcccccaagctggccagactgcactacagtggaagcatcaatgcatggagcaccaaggagcccttcagctggatcaaggtggacctgctggcccccatgatcatccatgggatcaagacccagggggccagacagaagttcagcagcctgtacatcagccagttcatcatcatgtacagcctggatggcaagaagtggcagacctacagaggcaacagcacaggtaccctgatggtgttctttggaaatgtggacagctcaggtatcaagcacaacatcttcaacccccccatcatagcaagatacatcagactgcaccccacccactacagcatcagaagcaccctgagaatggagctgatgggctgtgacctgaacagctgcagcatgcccctgggcatggagagcaaggccatctcagatgctcagatcactgccagcagctacttcaccaacatgtttgccacctggagccccagcaaggccagactgcacctgcagggcagaagcaatgcctggagaccccaggtgaacaaccccaaggagtggctgcaggtggacttccagaagaccatgaaggtgacaggggttaccacccagggagtgaagagcctgctgaccagcatgtatgtaaaggagttcctgatcagcagcagccaggatggtcaccagtggaccctgttcttccagaatggaaaggtgaaggtgttccagggcaaccaggacagcttcaccccagtggtgaacagcctggacccccccctgctgaccagatacctgagaatccacccccagagctgggtgcaccagatagccctgagaatggaggtgctgggctgtgaagcccaggacctgtactgatttaaatctcgagccgggcggagtgtgttagtctctccagagggaggctggttccccagggaagcagagcctgtgtgcgggcagcagctgtgtgcgggcctgggggttgttaagtgcaattatttttaataaaaggggcatttggaaaaaaaaaaaaaaggtagcagtcgacagatgaattctgcagatctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaaaaaagagaaatgttctggcacctgcacttgcactggggacagcctattttgctagtttgttttgtttcgttttgttttgatggagagcgtatgttgtttaaacgcggccgcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 51 - DNA, 6314 bp, synthetic construct
  source 1..6314: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacggcgcgccacgcgttctaggagttaatttttaaaaagcagtcaaaagtccaagtgcccttgcgagcatttactctctctgtttgctctggttaataatctcaggagcacaaacattccttactagttctaggagttaatttttaaaaagcagtcaaaagtccaagtgcccttgcgagcatttactctctctgtttgctctggttaataatctcaggagcacaaacattccttactagttctagagcggccgccagtgtgctggaattcggcttttttagggctggaagctacctttgacatcatttcctctgcgaatgcatgtataatttctacagaacctattagaaaggatcacccagcctctgcttttgtacaactttcccttaaaaaactgccaatcccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactgagagtgctctagttctgcaatacaggacatgctataaaaatggaaagatgttgctttctgagagatcaggtggattcttgggcatttgcttaagcttggtaccgccaccatgcaaatagaactctccacctgcttctttctgtgccttttgaggttctgcttttctgcaactagaagatactacctgggtgcagtagaactgtcatgggactatatgcaatcagatctaggagaactgccagtggatgccagatttcctccaagggtgcccaaatcatttccattcaacacaagtgttgtgtacaaaaagaccctgtttgtggaattcacagatcaccttttcaacatagccaagcctagaccaccttggatgggtctgttgggtcctaccatccaggcagaagtttatgatactgtggtcattacacttaagaacatggctagccatccagtcagtcttcatgctgttggggtatcctactggaaagcttcagagggagcagaatatgatgaccagacaagtcaaagggagaaagaagatgacaaagtcttccctggtggaagccatacctatgtctggcaggtcctgaaagagaatgggccaatggccagtgacccactgtgccttacctactcttatcttagtcatgtggacctggtaaaagacttgaatagtggcctcattggagcactactggtatgtagagaagggagcttggctaaggaaaagacacagactttgcacaaatttatactactttttgctgtatttgatgaggggaaaagttggcacagtgaaacaaagaactccttgatgcaggatagggatgcagcaagtgctagggcttggccaaaaatgcacacagttaatgggtatgttaacagatcactgcctgggctgattggttgccacagaaaatctgtctattggcatgtgattggaatgggcacaactcctgaagtgcactcaatattccttgaaggtcacacatttcttgtgagaaaccatagacaggcttccttggaaatcagccccataaccttccttacagctcaaacactcttgatggaccttggacagtttctactgttttgtcatatctcttcccaccaacatgatggaatggaagcctatgtcaaagtagacagctgtcctgaggaaccacaactacagatgaaaaataatgaagaagcagaagactatgatgatgatcttactgattcagaaatggatgtggtcaggtttgatgatgataactctccttcctttatccaaattagatctgtagcaaagaagcatcctaaaacttgggtacattacattgctgcagaagaggaggactgggactatgcacccttggtcttggcaccagatgatagaagttataaaagtcaatatttgaacaatggcccccagagaattggtaggaagtacaaaaaagtcagatttatggcatacacagatgaaacatttaagacaagagaagctattcagcatgagtcagggatcttgggacctttactttatggtgaagttggagatacactgttgattatatttaagaatcaagcaagcagaccatataacatctaccctcatggtatcacagatgtaagacccttgtattcaagaagattacccaaaggtgtaaaacatttgaaggattttccaattctgccaggggagatattcaaatataaatggacagtgacagtagaggatgggccaactaaatcagatcctaggtgcctgaccaggtattactctagttttgttaatatggagagagatctagctagtggactcattggacctctcctcatctgctacaaagaatcagttgatcaacagggaaaccagataatgtcagataagaggaatgtcatcctgttttctgtatttgatgaaaacaggagctggtacctcacagaaaatatacaaagatttctccccaatcctgctggagtgcagcttgaagatccagaattccaagcttcaaacatcatgcacagcatcaatggctatgtatttgattccttgcagttgtcagtgtgtttgcatgaggtggcatactggtacattctatctattggagctcagactgacttcctttctgtgttcttctcaggatatacattcaaacacaaaatggtctatgaagacacactcaccctattcccattctcaggggaaacagtcttcatgtcaatggaaaacccaggtctatggattttggggtgccacaactcagactttagaaacagaggcatgacagctttactgaaggttagtagttgtgataagaacactggggattattatgaggactcttatgaagatatttctgcatacttgctgagtaaaaacaatgcaattgaaccaagaagcttctctcaaaaccccccagtcttgaaaagacatcaaagagaaataaccagaactactcttcagtctgatcaagaggaaattgactatgatgataccatatcagttgaaatgaagaaggaagattttgacatttatgatgaagatgaaaatcagagccccaggagctttcaaaagaaaacaagacactattttattgcagcagtggagaggctctgggattatggtatgagtagctccccacatgtactaaggaacagggcccagagtggctctgtcccacagttcaagaaagtagttttccaggaatttacagatggctcctttactcagcctttatacaggggagaactaaatgaacatttgggactcctagggccatatataagagcagaggttgaagataatatcatggtaactttcaggaatcaggcctctaggccctattccttctattctagccttatttcttatgaggaagatcagaggcaaggagcagagcctaggaaaaactttgttaagcctaatgaaaccaaaacatacttttggaaagtgcaacatcatatggcccccaccaaagatgaatttgactgcaaagcctgggcatatttctctgatgtggacttagaaaaagatgttcactcaggcctgataggaccacttctggtctgccacactaacacactgaacccagcccatgggagacaagtgactgtacaggaatttgcactgtttttcaccatctttgatgaaacaaaaagctggtacttcactgaaaatatggaaagaaactgcagggctccctgcaatatccagatggaagatccaacatttaaagagaattatagattccatgctatcaatggctacataatggatacactaccaggcttagtaatggctcaggatcaaaggattagatggtatctgctcagcatgggcagtaatgagaacatccattctattcatttctcaggacatgtattcactgtaagaaaaaaagaggagtataaaatggcactgtacaatctctatccaggtgtttttgagacagtagaaatgttaccttcaaaagcaggaatttggagagtggaatgccttattggtgagcatctacatgctggaatgagcacactttttctggtgtacagcaataagtgtcagacccctctgggaatggcaagtggacacattagagactttcagattacagcttcaggacaatatggacagtgggccccaaagctggccagacttcattattcagggtcaatcaatgcatggagcaccaaggagccttttagctggatcaaggtggatctgttggcaccaatgattattcatggcatcaagactcagggagccagacagaagttctcaagcctctacatctctcagtttatcatcatgtattcccttgatgggaagaagtggcagacttatagaggaaatagtacaggaaccttaatggtcttctttgggaatgtggattcaagtgggataaaacacaatatttttaaccctccaattattgctaggtacatcaggttgcaccccactcattatagcattaggagcactcttagaatggagttgatgggctgtgatttaaatagttgcagtatgccattgggaatggagagtaaagcaatatcagatgcacagattactgcatcatcttactttactaatatgtttgcaacttggtctccatcaaaagcaagacttcacctccaagggaggtctaatgcttggagacctcaggtgaataatccaaaagagtggctgcaagtggacttccagaagacaatgaaagtcacaggtgtgaccactcagggtgtaaaatctctgcttaccagcatgtatgttaaggagttcctcatctccagcagtcaagatggacatcagtggactctcttttttcagaatggtaaagtaaaggtttttcagggaaatcaagactccttcacaccagtggttaacagtctagacccaccattactgactaggtaccttagaattcacccacagagttgggtgcaccagattgccctgaggatggaggttctgggctgtgaagcacaggacctctactgatttaaatctcgagccgggcggagtgtgttagtctctccagagggaggctggttccccagggaagcagagcctgtgtgcgggcagcagctgtgtgcgggcctgggggttgttaagtgcaattatttttaataaaaggggcatttggaaaaaaaaaaaaaaggtagcagtcgacagatgaattctgcagatctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaaaaaagagaaatgttctggcacctgcacttgcactggggacagcctattttgctagtttgttttgtttcgttttgttttgatggagagcgtatgttgtttaaacgcggccgcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 52 - DNA, 6314 bp, synthetic construct
  source 1..6314: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacggcgcgccacgcgttctaggagttaatttttaaaaagcagtcaaaagtccaagtgcccttgcgagcatttactctctctgtttgctctggttaataatctcaggagcacaaacattccttactagttctaggagttaatttttaaaaagcagtcaaaagtccaagtgcccttgcgagcatttactctctctgtttgctctggttaataatctcaggagcacaaacattccttactagttctagagcggccgccagtgtgctggaattcggcttttttagggctggaagctacctttgacatcatttcctctgcgaatgcatgtataatttctacagaacctattagaaaggatcacccagcctctgcttttgtacaactttcccttaaaaaactgccaatcccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactgagagtgctctagttctgcaatacaggacatgctataaaaatggaaagatgttgctttctgagagatcaggtggattcttgggcatttgcttaagcttggtaccgccaccatgcaaatagagctctccacctgcttctttctgtgccttttgaggttctgcttttctgccacaaggagatactacctgggtgcagtggaactgtcatgggactatatgcaaagtgacctaggagagctgccagtggatgcaaggtttcctcctagagtgcccaaaagttttccattcaacacatcagttgtgtacaaaaagacactgtttgtagaattcacagatcaccttttcaacatagcaaagcccaggccaccatggatgggtctgctaggtcctaccatccaggcagaggtttatgatactgtggtcattacacttaagaacatggcttcacatcctgtcagtcttcatgctgtgggtgtatcctactggaaagcttcagagggagcagaatatgatgaccagactagtcaaagagagaaagaagatgataaagtcttcccaggtggatcccatacctatgtctggcaggtcctgaaagagaatggtcctatggccagtgatcctctgtgccttacttacagttatcttagtcatgttgacctggtaaaagacttgaattcaggactcattggagcactattggtatgtagagaagggtctttagctaaggaaaagacacagaccttgcacaaatttatactactttttgcagtatttgatgaagggaaaagttggcacagtgaaacaaagaactccttgatgcaggatagggatgcagcatctgctagggcctggcccaaaatgcacacagtcaatgggtatgtgaacaggtctctgcctggtctgattggatgccacagaaaatcagtatattggcatgtgataggaatgggcacaactcctgaggtgcactcaatattcttagaaggtcacacatttcttgtaagaaaccatagacaggcatccttggaaatctctcccataacattccttacagctcaaaccctcttgatggaccttggacagtttctactgttttgtcatatctcttcccaccaacatgatgggatggaagcatatgtcaaagtagacagctgtccagaggaaccacaactacagatgaaaaataatgaagaagcagaagactatgatgatgatcttactgattcagaaatggatgtagtcaggtttgatgatgataactctccttcctttatccaaattaggtctgttgccaagaagcatcctaaaacttgggtacattacattgctgcagaagaggaggactgggactatgcaccattagtactagcacctgatgacagaagttataaaagtcaatatttgaacaatggaccccagagaattgggaggaagtacaaaaaagtcagatttatggcatacactgatgagacatttaagactagagaagctattcagcatgaatcaggaatcttgggacctttactttatggggaagtaggagacacactgttgattatatttaagaatcaagcaagcagaccatataacatctacccacatggaatcacagatgttagacccttgtattcaaggaggttaccaaaaggtgtaaaacatttgaaggattttcctattctgccaggtgaaatattcaaatataaatggactgtgactgttgaagatggaccaactaaatcagaccccaggtgcctgaccaggtattactctagttttgttaatatggagagagatctagcttcaggactcataggccctctcctcatctgctacaaagaatcagtagatcaacagggaaaccagataatgtcagacaagaggaatgtaatcctgttttctgtatttgatgaaaacagaagctggtacctcacagagaatatacaaaggtttctccccaatcctgcaggagtgcagcttgaggatcctgagttccaagcttccaacatcatgcacagcatcaatgggtatgtgtttgattctttgcagttgtctgtttgtttgcatgaagtggcatactggtacattctatctattggtgctcagactgacttcctttctgtattcttctcaggctatactttcaaacacaaaatggtctatgaagacaccctcaccctattcccattctctggggaaactgtgttcatgtccatggaaaaccctggactatggattctgggctgccacaactctgactttagaaacagaggcatgactgctttactgaaggtttcaagttgtgacaagaacactggtgattattatgaagactcctatgaagatattagtgcctacttgctgagtaaaaacaatgcaatagaaccaaggagcttctctcaaaaccccccagtcttgaaaaggcatcaaagagaaataaccagaactactcttcagtcagatcaagaggaaattgactatgatgataccataagtgttgaaatgaagaaggaagattttgacatttatgatgaggatgaaaatcagagccccaggagctttcaaaagaaaacaagacactattttatagctgctgtagagagactctgggattatgggatgagttcatcccctcatgtactaaggaacagggcccagtcaggttcagtcccccagttcaagaaagttgtcttccaggaatttacagatgggtcctttactcagcctttatacaggggagaactaaatgaacatttgggactcctgggaccatatataagagcagaagttgaagataatatcatggtaactttcaggaatcaggcctctagaccctattccttctattctagccttatttcttatgaagaagatcagaggcaaggagcagaacctagaaaaaactttgtcaagcctaatgaaactaaaacttacttttggaaagtgcaacatcatatggctcccactaaagatgagtttgattgcaaagcctgggcatatttctcagatgttgacctagagaaagatgtacactcaggactgattggacctcttctggtctgccacactaacacactgaaccctgctcatggtagacaagtgactgtacaggaatttgccctgtttttcaccatctttgatgagacaaaaagctggtacttcacagaaaatatggaaagaaactgcagggctccctgcaatatccagatggaagatcctacctttaaagagaattataggttccatgcaatcaatggctacataatggatacactacctgggttagtaatggctcaggatcaaaggattaggtggtatctgctcagcatgggctccaatgagaacatccattccattcatttctcaggacatgtgttcacagttaggaaaaaagaggagtataaaatggcactgtacaatctctatccaggtgtttttgaaacagtagaaatgttacccagtaaagctggaatttggagggtggagtgccttataggtgagcatctacatgcaggaatgagcacactttttctggtgtacagcaataagtgtcagacacccctgggaatggcctcaggacacattagggactttcagattacagccagtggacaatatggtcagtgggccccaaagctggccagacttcattatagtggttcaatcaatgcttggagcactaaggagcccttttcttggatcaaggtggatctgctggctcccatgattattcatggcatcaagacacagggtgctaggcagaagttcagcagtctctacatctctcagtttatcatcatgtattcacttgatgggaagaagtggcagacttatagaggaaattccacaggaaccttaatggtcttctttggaaatgtggattcatctggaataaaacacaatatttttaaccctccaattattgctagatacatcaggttgcacccaactcattatagcattagaagcactcttagaatggagttgatgggctgtgatttaaatagttgctccatgcccttgggaatggagagtaaagcaatatcagatgcccagattacagcttcatcatactttaccaatatgtttgcaacatggtctccatcaaaagcaagacttcacctccaagggagaagtaatgcctggagacctcaggtgaataatccaaaagagtggctgcaagtggacttccagaagactatgaaagtcacaggagtaacaactcagggagttaaatctctgcttaccagcatgtatgtaaaggagttcctcatctccagcagtcaagatgggcatcagtggactctcttttttcagaatggaaaagtaaaggtttttcagggaaatcaagactccttcacaccagtggttaacagtctggaccctcccttactgactagataccttaggattcaccctcagagttgggtgcaccagattgcactgagaatggaggttctgggctgtgaggcacaggacctctactgatttaaatctcgagccgggcggagtgtgttagtctctccagagggaggctggttccccagggaagcagagcctgtgtgcgggcagcagctgtgtgcgggcctgggggttgttaagtgcaattatttttaataaaaggggcatttggaaaaaaaaaaaaaaggtagcagtcgacagatgaattctgcagatctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaaaaaagagaaatgttctggcacctgcacttgcactggggacagcctattttgctagtttgttttgtttcgttttgttttgatggagagcgtatgttgtttaaacgcggccgcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 53 - DNA, 6314 bp, synthetic construct
  source 1..6314: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacggcgcgccacgcgttctaggagttaatttttaaaaagcagtcaaaagtccaagtgcccttgcgagcatttactctctctgtttgctctggttaataatctcaggagcacaaacattccttactagttctaggagttaatttttaaaaagcagtcaaaagtccaagtgcccttgcgagcatttactctctctgtttgctctggttaataatctcaggagcacaaacattccttactagttctagagcggccgccagtgtgctggaattcggcttttttagggctggaagctacctttgacatcatttcctctgcgaatgcatgtataatttctacagaacctattagaaaggatcacccagcctctgcttttgtacaactttcccttaaaaaactgccaatcccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactgagagtgctctagttctgcaatacaggacatgctataaaaatggaaagatgttgctttctgagagatcaggtggattcttgggcatttgcttaagcttggtaccgccaccatgcaaatagagctctccacctgcttctttctgtgccttttgaggttctgctttagtgcaacaagaagatactacctgggtgcagtagaactgtcatgggactatatgcaatctgatttaggggagctgcctgtggatgcaagatttccacccagggtgcctaaaagctttcccttcaacacttcagttgtgtacaaaaagaccctgtttgtagaattcactgatcaccttttcaacattgctaagccaaggccaccctggatgggtctgcttggtcctaccatccaggcagaagtttatgatactgtggtcattacacttaagaacatggcttcccatccagtgagtcttcatgctgtgggtgtatcctactggaaagcttctgagggagctgaatatgatgaccagaccagtcaaagggagaaagaagatgacaaagtcttcccaggtggaagccatacctatgtgtggcaggtcctgaaagagaatggtccaatggcctctgatcctctgtgccttacctactcctatctttcacatgttgacctggtaaaagacttgaattcaggcctcattggggccctactagtttgtagagaagggtctctggctaaggaaaagactcagacattgcacaaatttatactactttttgctgtatttgatgaggggaaaagttggcactcagagacaaagaactccttgatgcaggatagagatgctgcttcagctagagcatggcctaaaatgcacactgtcaatggatatgtaaacagaagcctgccaggtctgattggttgccacaggaaatctgtctattggcatgtgattggcatgggcaccactccagaagtgcactcaatattcttagagggtcacacatttcttgtaagaaaccatagacaggcttccttggaaatctcacctataaccttccttacagctcaaaccctcttgatggacctgggacagtttctactgttttgtcatatctcttcccaccaacatgatggaatggaagcttatgtcaaagtagacagctgtccagaggaaccccaactacagatgaaaaataatgaagaagctgaagactatgatgatgatcttactgatagtgaaatggatgtagtcaggtttgatgatgacaactctccttcctttatccaaattaggtcagttgctaagaagcatcctaaaacttgggtacattacattgcagcagaagaggaggactgggactatgccccattagtcctagcccctgatgatagaagttataaaagtcaatatttgaacaatggaccacagagaattggtaggaagtacaaaaaagtcagatttatggcatacactgatgagacctttaagacaagggaagctattcagcatgaatctggaatcttgggacctttactttatggagaagttggggacacactgttgattatatttaagaatcaagcaagcagaccatataacatctacccccatggaatcactgatgtcaggcccttgtattcaagaaggttacctaaaggtgtaaaacatttgaaggattttcctattctgccaggtgaaatattcaaatataaatggacagtgactgtagaagatggcccaactaaaagtgatcccaggtgcctgaccagatattactctagttttgtcaatatggagagagatctagcttcaggactcattggtcctctcctcatctgctacaaagaatcagtagatcaacagggaaaccagataatgagtgataagaggaatgtcatcctgtttagtgtatttgatgaaaacagaagctggtacctcacagaaaatatacaaagatttctccccaatccagctggggtgcagttggaggatccagaattccaagcaagtaacatcatgcacagcatcaatgggtatgtatttgactctttgcagttgtcagtatgtttgcatgaagtggcatactggtacattctatcaattggggcccagactgatttccttagtgtcttcttctctggttatacattcaaacacaaaatggtctatgaagacaccctcaccctattcccattctcaggggaaactgtcttcatgagtatggaaaacccaggtctatggattttggggtgccacaactcagactttaggaacagaggcatgactgcattactgaaggtttcaagttgtgataagaacactggtgattattatgaagacagttatgaggatatttcagcatacttgctgagtaaaaacaatgcaatagaaccaagaagcttctctcaaaacccaccagtcttgaaaagacatcaaagagaaataaccaggactactcttcagtctgatcaagaggaaattgattatgatgacaccatatcagttgaaatgaagaaggaagattttgatatttatgatgaggatgaaaatcagagcccaaggagctttcaaaagaaaaccagacactattttattgcagcagtagagagactctgggattatgggatgagttcatccccccatgttctaagaaacagggcacagagtggcagtgtcccacagttcaagaaagtagttttccaggaatttacagatggttcctttactcagccattatacaggggagaactaaatgaacatttgggactcttagggccatatataagagctgaagttgaagataatatcatggtaactttcaggaatcaggcctctagaccatattccttctatagtagccttatttcttatgaggaagatcagaggcaaggagcagagcctaggaaaaactttgttaagcctaatgaaacaaaaacttacttttggaaagtgcaacatcatatggcacccaccaaagatgagtttgactgcaaagcctgggcctatttctctgatgttgacttagaaaaagatgtgcactctgggctgattggaccacttctggtctgccacactaacacactgaacccagcacatggcagacaagtgactgtccaggaatttgctctgtttttcaccatctttgatgagacaaaaagctggtacttcactgaaaatatggaaaggaactgcagggctccctgcaatatccagatggaagatccaacctttaaagagaattatagattccatgccatcaatgggtacataatggatacactacctggcttagtaatggctcaggatcaaaggattaggtggtatctgctcagcatgggctccaatgaaaacatccatagcattcatttcagtggacatgttttcactgtaaggaaaaaagaggagtataaaatggcactgtacaatctctatccaggagtttttgagacagttgaaatgttaccatctaaagcaggaatttggagggttgaatgccttattggggaacatctacatgcaggtatgagcacactttttctggtgtacagcaataagtgtcagactccactgggaatggcatctggccacattagagattttcagattactgcatcagggcaatatggacagtgggccccaaagctggccagacttcattatagtggatcaatcaatgcttggagcaccaaggagcccttttcttggatcaaggtggatctgcttgcacccatgattattcatgggatcaagacccagggtgccagacagaagttcagcagcctctacatctctcagtttatcatcatgtatagccttgatggaaagaagtggcagacttataggggaaatagcacaggaaccttaatggtcttctttggtaatgttgattcaagtgggataaaacacaatatttttaaccctccaattatagctagatacatcagattgcaccccactcattatagcattaggagcactcttaggatggagttgatgggctgtgatttaaatagttgcagtatgcccttgggaatggagagtaaagcaatatcagatgctcagattactgcatcaagctactttactaatatgtttgctacatggtctccatcaaaagctagacttcacctccaagggagatccaatgcttggagacctcaggtgaataatccaaaagagtggctgcaagtggacttccagaagactatgaaagtcacaggggtaacaacccagggagtaaaatctctgcttaccagcatgtatgtcaaggagttcctcatctccagctcacaagatgggcatcagtggactctcttttttcagaatgggaaagtaaaggtttttcagggaaatcaagactccttcacaccagtggtgaactctttggacccacctttactgactagataccttaggattcacccacagagttgggtgcaccagatagctctgagaatggaggttctgggctgtgaggcccaggacctctactgatttaaatctcgagccgggcggagtgtgttagtctctccagagggaggctggttccccagggaagcagagcctgtgtgcgggcagcagctgtgtgcgggcctgggggttgttaagtgcaattatttttaataaaaggggcatttggaaaaaaaaaaaaaaggtagcagtcgacagatgaattctgcagatctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaaaaaagagaaatgttctggcacctgcacttgcactggggacagcctattttgctagtttgttttgtttcgttttgttttgatggagagcgtatgttgtttaaacgcggccgcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 54 - DNA, 60 bp, unidentified
  source 1..60: mol_type = unassigned DNA
atggagttcgggctgagctgggtctttctggtggccctgctgaagggagtccagtgcgag

SEQ ID NO: 55 - DNA, 60 bp, Homo sapiens
  source 1..60: mol_type = genomic DNA
atgtaccgtatgcagctgctgtcctgcatcgcactgtcactggcactggttaccaactca

SEQ ID NO: 56 - DNA, 60 bp, synthetic construct
  source 1..60: mol_type = other DNA
atgtaccgtatgcagctgctgctgctgatcgcactgtcactggcactggttaccaactca

SEQ ID NO: 57 - DNA, 69 bp, unidentified
  source 1..69: mol_type = unassigned DNA
atggggaagaacaaactccttcatccaagtctggttcttctcctcttggtcctcctgcccacagatgcc

SEQ ID NO: 58 - DNA, 75 bp, unidentified
  source 1..75: mol_type = unassigned DNA
atgagaaaagacagacttcttcatttatgtcttgtgctacttcttatcctgctttctgccagtgactcaaactct

SEQ ID NO: 59 - skipped

SEQ ID NO: 60 - protein, 20 aa, unidentified
MEFGLSWVFLVALLKGVQCE

SEQ ID NO: 61 - protein, 20 aa, Homo sapiens
MYRMQLLSCIALSLALVTNS

SEQ ID NO: 62 - protein, 20 aa, synthetic construct
MYRMQLLLLIALSLALVTNS

SEQ ID NO: 63 - protein, 23 aa, unidentified
MGKNKLLHPSLVLLLLVLLPTDA

SEQ ID NO: 64 - protein, 25 aa, unidentified
MRKDRLLHLCLVLLLILLSASDSNS

SEQ ID NO: 65 - protein, 21 aa, synthetic construct
MWWRLWWLLLLLLLLWPMVWA

SEQ ID NO: 66 - protein, 11 aa, unidentified
LLLLAVLCLGT

SEQ ID NO: 67 - DNA, 72 bp, unidentified
  source 1..72: mol_type = unassigned DNA
atgccgtcttctgtctcgtggggcatcctcctgctggcaggcctgtgctgcctggtccctgtctccctggct

SEQ ID NO: 68 - protein, 32 aa, Rattus sp.
MLRGPGPGRLLLLAVLCLGTSVRCTETGKSKR

SEQ ID NO: 69 - protein, 38 aa, Rattus sp.
MLRGPGPGRLLLLAVLCLGTSVRCTETGKSKRLALQIV

SEQ ID NO: 70 - protein, 26 aa, Homo sapiens
MLRGPGPGLLLLAVQCLGTAVPSTGA

SEQ ID NO: 71 - protein, 31 aa, unidentified
MRRGALTGLLLVLCLSVVLRAAPSATSKKRR

SEQ ID NO: 72 - DNA, 72 bp, Homo sapiens
  source 1..72: mol_type = genomic DNA
atgggaatcccaatggggaagtcgatgctggtgcttctcaccttcttggccttcgcctcgtgctgcattgct

SEQ ID NO: 73 - DNA, 96 bp, Rattus sp.
  source 1..96: mol_type = genomic DNA
atgctcaggggtccgggacccgggcggctgctgctgctagcagtcctgtgcctggggacatcggtgcgctgcaccgaaaccgggaagagcaagagg

SEQ ID NO: 74 - DNA, 114 bp, Rattus sp.
  source 1..114: mol_type = genomic DNA
atgctcaggggtccgggacccgggcggctgctgctgctagcagtcctgtgcctggggacatcggtgcgctgcaccgaaaccgggaagagcaagaggcaggctcagcaaatcgtg

SEQ ID NO: 75 - DNA, 78 bp, Homo sapiens
  source 1..78: mol_type = genomic DNA
atgcttaggggtccggggcccgggctgctgctgctggccgtccagtgcctggggacagcggtgccctccacgggagcc

SEQ ID NO: 76 - DNA, 93 bp, unidentified
  source 1..93: mol_type = unassigned DNA
atgcgccggggggccctgaccgggctgctcctggtcctgtgcctgagtgttgtgctacgtgcagccccctctgcaacaagcaagaagcgcagg

SEQ ID NO: 77 - protein, 17 aa, Rattus sp.
MTPLLLLAVLCLGTALA

SEQ ID NO: 78 - protein, 22 aa, Homo sapiens
MLSFVDTRTLLLLAVTLCLATC

SEQ ID NO: 79 - DNA, 130 bp, synthetic construct
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 80 - DNA, 130 bp, synthetic construct
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 81 - DNA, 124 bp, synthetic construct
  source 1..124: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggg

SEQ ID NO: 82 - DNA, 130 bp, synthetic construct; identical to SEQ ID NO: 80
  source 1..130: mol_type = other DNA

SEQ ID NO: 83 - DNA, 167 bp, synthetic construct
  source 1..167: mol_type = other DNA
aggaacccctagtgatggagttggccactccctctctgcgcgctcgctcgctcactgaggcaatttgataaaaatcgtcaaattataaacaggctttgcctgtttagcctcagtgagcgagcgagcgcgcagagagggagtggccaactccatcactaggggttcct

SEQ ID NO: 84 - DNA, 143 bp, synthetic construct
  source 1..143: mol_type = other DNA
aggaacccctagtgatggagttggccactccctctctgcgcgctcgctcgctcactgagggataaaaatccaggctttgcctgcctcagtgagcgagcgagcgcgcagagagggagtggccaactccatcactaggggttcct

SEQ ID NO: 85 - DNA, 143 bp, synthetic construct; identical to SEQ ID NO: 84
  source 1..143: mol_type = other DNA

SEQ ID NO: 86 - DNA, 180 bp, synthetic construct
  source 1..180: mol_type = other DNA
aggttaatttttaaaaagcagtcaaaagtccaagtggcccttggcagcatttactctctctgtttgctctggttaataatctcaggagcacaaacattccccctgttcaaacatgtcctaatactctgtctctgcaagggtcatcagtagttttccatcttactcaacatcctcccagtg

SEQ ID NO: 87 - DNA, 402 bp, synthetic construct
  source 1..402: mol_type = other DNA
caggctttcactttctcgccaacttacaaggcctttctgtgtaaacaatacctgaacctttaccccgttgcccggcaacggccaggtctgtgccaagtgtttgaggttaatttttaaaaagcagtcaaaagtccaagtggcccttggcagcatttactctctctgtttgctctggttaataatctcaggagcacaaacattccggcccgggaggcgccctttggaccttttgcaatcctggcgcactgaacccttgacccctgccctgcagcccccgcagcttgctgtttgcccactctatttgcccagccccagccctggagagtcctttagcagggcaaagtgcaacataggcagaccttaagggatgactcagtaacagataagctttgtgtgcctgca

SEQ ID NO: 88 - DNA, 305 bp, synthetic construct
  source 1..305: mol_type = other DNA
aggttaatttttaaaaagcagtcaaaagtccaagtggcccttggcagcatttactctctctgtttgctctggttaataatctcaggagcacaaacattcctgtaccggcccgggaggcgccctttggaccttttgcaatcctggcgcactgaacccttgacccctgccctgcagcccccgcagcttgctgtttgcccactctatttgcccagccccagccctggagagtcctttagcagggcaaagtgcaacataggcagaccttaagggatgactcagtaacagataagctttgtgtgcctgca

SEQ ID NO: 89 - DNA, 103 bp, synthetic construct
  source 1..103: mol_type = other DNA
caggctttcactttctcgccaacttacaaggcctttctgtgtaaacaatacctgaacctttaccccgttgcccggcaacggccaggtctgtgccaagtgtttg

SEQ ID NO: 90 - DNA, 212 bp, synthetic construct
  source 1..212: mol_type = other DNA
aggttaatttttaaaaagcagtcaaaagtccaagtggcccttggcagcatttactctctctgtttgctctggttaataatctcaggagcacaaacattccggcccgggaggcgccctttggaccttttgcaatcctggcgcactgaacccttgacccctgccctgcagcccccgcagcttgctgtttgcccactctatttgcccagccccag

SEQ ID NO: 91 - DNA, 283 bp, synthetic construct
  source 1..283: mol_type = other DNA
aggttaatttttaaaaagcagtcaaaagtccaagtggcccttggcagcatttactctctctgtttgctctggttaataatctcaggagcacaaacattccccctgttcaaacatgtcctaatactctgtctctgcaagggtcatcagtagttttccatcttactcaacatcctcccagtgagtcatatgtttgctcactgaaggttactagttaacaggcatcccttaaacaggatataaaaggacttcagcaggactgctcgaaacatcccactcagccacc

SEQ ID NO: 92 - DNA, 565 bp, synthetic construct
  source 1..565: mol_type = other DNA
aggttaatttttaaaaagcagtcaaaagtccaagtggcccttggcagcatttactctctctgtttgctctggttaataatctcaggagcacaaacattccccctgttcaaacatgtcctaatactctgtctctgcaagggtcatcagtagttttccatcttactcaacatcctcccagtgagtcatatgtttgctcactgaaggttactagttaacaggcatcccttaaacaggatataaaaggacttcagcaggactgctcgaaacatcccacttcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgaacggtggagggcagtgtagtctgagcagtactcgttgctgccgcgcgcgccaccagacataatagctgacagactaacagactgttcctttccatgggtcttttctgcaggccacc

SEQ ID NO: 93 - DNA, 478 bp, synthetic construct
  source 1..478: mol_type = other DNA
caggctttcactttctcgccaacttacaaggcctttctgtgtaaacaatacctgaacctttaccccgttgcccggcaacggccaggtctgtgccaagtgtttgaggttaatttttaaaaagcagtcaaaagtccaagtggcccttggcagcatttactctctctgtttgctctggttaataatctcaggagcacaaacattccggcccgggaggcgccctttggaccttttgcaatcctggcgcactgaacccttgacccctgccctgcagcccccgcagcttgctgtttgcccactctatttgcccagccccagccctggagagtcctttagcagggcaaagtgcaacataggcagaccttaagggatgactcagtaacagataagctttgtgtgcctgcagggcatataaaacaggggcaaggcacagactcatagcagagcaatcaccaccaagcctggaataactgcagccacc

SEQ ID NO: 94 - DNA, 381 bp, synthetic construct
  source 1..381: mol_type = other DNA
aggttaatttttaaaaagcagtcaaaagtccaagtggcccttggcagcatttactctctctgtttgctctggttaataatctcaggagcacaaacattcctgtaccggcccgggaggcgccctttggaccttttgcaatcctggcgcactgaacccttgacccctgccctgcagcccccgcagcttgctgtttgcccactctatttgcccagccccagccctggagagtcctttagcagggcaaagtgcaacataggcagaccttaagggatgactcagtaacagataagctttgtgtgcctgcagggcatataaaacaggggcaaggcacagactcatagcagagcaatcaccaccaagcctggaataactgcagccacc

SEQ ID NO: 95 - DNA, 379 bp, synthetic construct
  source 1..379: mol_type = other DNA
caggctttcactttctcgccaacttacaaggcctttctgtgtaaacaatacctgaacctttaccccgttgcccggcaacggccaggtctgtgccaagtgtttgccgatgacctaatgattctgagcttggcaaaggtcttatctcccagctcgcccaggcccagtgttccaggaatgtgacctttgctgcagcagccgctggagggggcagaggggatgggctggaggttgagcaaacagagcagcagaaaaggcagttcctcttctccagtgccctccttccctgtctctgcctctccctcccttcctcaggcatcagagcggagacttcagggagaccagagcccagcttgccaggcactgagctagaagccctgcc

SEQ ID NO: 96 - DNA, 288 bp, synthetic construct
  source 1..288: mol_type = other DNA
aggttaatttttaaaaagcagtcaaaagtccaagtggcccttggcagcatttactctctctgtttgctctggttaataatctcaggagcacaaacattccggcccgggaggcgccctttggaccttttgcaatcctggcgcactgaacccttgacccctgccctgcagcccccgcagcttgctgtttgcccactctatttgcccagccccaggggcatataaaacaggggcaaggcacagactcatagcagagcaatcaccaccaagcctggaataactgcagccacc

SEQ ID NO: 97 - DNA, 741 bp, synthetic construct
  source 1..741: mol_type = other DNA
tctaggagttaatttttaaaaagcagtcaaaagtccaagtgcccttgcgagcatttactctctctgtttgctctggttaataatctcaggagcacaaacattccttactagttctaggagttaatttttaaaaagcagtcaaaagtccaagtgcccttgcgagcatttactctctctgtttgctctggttaataatctcaggagcacaaacattccttactagttctagagcggccgccagtgtgctggaattcggcttttttagggctggaagctacctttgacatcatttcctctgcgaatgcatgtataatttctacagaacctattagaaaggatcacccagcctctgcttttgtacaactttcccttaaaaaactgccaatcccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactgagagtgctctagttctgcaatacaggacatgctataaaaatggaaagatgttgctttctgagagat

SEQ ID NO: 98 - DNA, 757 bp, synthetic construct
  source 1..757: mol_type = other DNA
tctaggagttaatttttaaaaagcagtcaaaagtccaagtgcccttgcgagcatttactctctctgtttgctctggttaataatctcaggagcacaaacattccttactagttctaggagttaatttttaaaaagcagtcaaaagtccaagtgcccttgcgagcatttactctctctgtttgctctggttaataatctcaggagcacaaacattccttactagttctagagcggccgccagtgtgctggaattcggcttttttagggctggaagctacctttgacatcatttcctctgcgaatgcatgtataatttctacagaacctattagaaaggatcacccagcctctgcttttgtacaactttcccttaaaaaactgccaatcccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctgtgaaaataccatcccagggtatttatagctggggttaatttataactgagagtgctctagttctgcaatacaggacatgctataaaaatggaaagatgttgctttctgagagatcagcttacatgtg

SEQ ID NO: 99 - DNA, 754 bp, synthetic construct
  source 1..754: mol_type = other DNA
tctaggagttaatttttaaaaagcagtcaaaagtccaagtgcccttgcgagcatttactctctctgtttgctctggttaataatctcaggagcacaaacattccttactagttctaggagttaatttttaaaaagcagtcaaaagtccaagtgcccttgcgagcatttactctctctgtttgctctggttaataatctcaggagcacaaacattccttactagttctagagcggccgccagtgtgctggaattcggcttttttagggctggaagctacctttgacatcatttcctctgcgaatgcatgtataatttctacagaacctattagaaaggatcacccagcctctgcttttgtacaactttcccttaaaaaactgccaatcccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactgagagtgctctagttctgcaatacaggacatgctataaaaatggaaagatgttgctttctgagagatcagcttacatgtg

SEQ ID NO: 100 - DNA, 208 bp, synthetic construct
  source 1..208: mol_type = other DNA
aggaacccctagtgatggagttggccactccctctctgggattgggattgcgcgctcgctcgcgggattgggattgggattgggattgggattgggattgataaaaatcaatcccaatcccaatcccaatcccaatcccaatcccgcgagcgagcgcgcaatcccaatcccagagagggagtggccaactccatcactaggggttcct

SEQ ID NO: 101 - DNA, 199 bp, synthetic construct
  source 1..199: mol_type = other DNA
aggaacccctagtgatggagttggccactccctctctgcgcgctcgctcgctcgggattgggattgggattgggattgggattgggattgataaaaatcaatcccaatcccaatcccaatcccaatcccaatcccgcgagcgagcgcgcaggagagggagtggccaactccatcactaggggttcctaagcttattata

SEQ ID NO: 102 - DNA, 154 bp, synthetic construct
  source 1..154: mol_type = other DNA
aggaacccctagtgatggagttggccactccctctctgcgcgctcgctcgctcactgagggcgcctataaagataaaaatccaggctttgcctgcctcagttagcgagcgagcgcgcagagagggagtggccaactccatcactaggggttcct

SEQ ID NO: 103 - DNA, 127 bp, synthetic construct
  source 1..127: mol_type = other DNA
ctagtgatggagttggccactccctctctgcgcgctcgctcgctcactgagggataaaaatccaggctttgcctgcctcagtgagcgagcgagcgcgcagagagggagtggccaactccatcactag

SEQ ID NO: 104 - DNA, 145 bp, synthetic construct
  source 1..145: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 105 - DNA, 145 bp, synthetic construct
  source 1..145: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 106 - DNA, 251 bp, synthetic construct
  source 1..251: mol_type = other DNA
gtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatc

SEQ ID NO: 107 - DNA, 22 bp, synthetic construct
  source 1..22: mol_type = other DNA
gggggcgactcagatcccagcc

SEQ ID NO: 108 - DNA, 20 bp, synthetic construct
  source 1..20: mol_type = other DNA
aggacgctgtggtttctgag

SEQ ID NO: 109 - DNA, 20 bp, synthetic construct
  source 1..20: mol_type = other DNA
aggtcaccccagttatcgga

SEQ ID NO: 110-143 - skipped (34 entries)

SEQ ID NO: 144 - DNA, 275 bp, synthetic construct
  source 1..275: mol_type = other DNA
ggcccgggaggcgccctttggaccttttgcaatcctggcgcactgaacccttgacccctgccctgcagcccccgcagcttgctgtttgcccactctatttgcccagccccagccctggagagtcctttagcagggcaaagtgcaacataggcagaccttaagggatgactcagtaacagataagctttgtgtgcctgcagggcatataaaacaggggcaaggcacagactcatagcagagcaatcaccaccaagcctggaataactgcagccacc

SEQ ID NO: 145 - DNA, 284 bp, synthetic construct
  source 1..284: mol_type = other DNA
tcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgaacggtggagggcagtgtagtctgagcagtactcgttgctgccgcgcgcgccaccagacataatagctgacagactaacagactgttcctttccatgggtcttttctgcag

SEQ ID NO: 146 - DNA, 636 bp, synthetic construct
  source 1..636: mol_type = other DNA
aggttaatttttaaaaagcagtcaaaagtccaagtggcccttggcagcatttactctctctgtttgctctggttaataatctcaggagcacaaacattcctgtaccggcccgggaggcgccctttggaccttttgcaatcctggcgcactgaacccttgacccctgccctgcagcccccgcagcttgctgtttgcccactctatttgcccagccccagccctggagagtcctttagcagggcaaagtgcaacataggcagaccttaagggatgactcagtaacagataagctttgtgtgcctgcagggcatataaaacaggggcaaggcacagactcatagcagagtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgaacggtggagggcagtgtagtctgagcagtactcgttgctgccgcgcgcgccaccagacataatagctgacagactaacagactgttcctttccatgggtcttttctgcaggccacc

SEQ ID NO: 147 - DNA, 733 bp, synthetic construct
  source 1..733: mol_type = other DNA
caggctttcactttctcgccaacttacaaggcctttctgtgtaaacaatacctgaacctttaccccgttgcccggcaacggccaggtctgtgccaagtgtttgaggttaatttttaaaaagcagtcaaaagtccaagtggcccttggcagcatttactctctctgtttgctctggttaataatctcaggagcacaaacattccggcccgggaggcgccctttggaccttttgcaatcctggcgcactgaacccttgacccctgccctgcagcccccgcagcttgctgtttgcccactctatttgcccagccccagccctggagagtcctttagcagggcaaagtgcaacataggcagaccttaagggatgactcagtaacagataagctttgtgtgcctgcagggcatataaaacaggggcaaggcacagactcatagcagagtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgaacggtggagggcagtgtagtctgagcagtactcgttgctgccgcgcgcgccaccagacataatagctgacagactaacagactgttcctttccatgggtcttttctgcaggccacc

SEQ ID NO: 148 - DNA, 653 bp, synthetic construct
  source 1..653: mol_type = other DNA
caggctttcactttctcgccaacttacaaggcctttctgtgtaaacaatacctgaacctttaccccgttgcccggcaacggccaggtctgtgccaagtgtttgccgatgacctaatgattctgagcttggcaaaggtcttatctcccagctcgcccaggcccagtgttccaggaatgtgacctttgctgcagcagccgctggagggggcagaggggatgggctggaggttgagcaaacagagcagcagaaaaggcagttcctcttctccagtgccctccttccctgtctctgcctctccctcccttcctcaggcatcagagcggagacttcagggagaccagagcccagcttgccaggcactgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgaacggtggagggcagtgtagtctgagcagtactcgttgctgccgcgcgcgccaccagacataatagctgacagactaacagactgttcctttccatgggtcttttctgcaggccacc

SEQ ID NO: 149 - DNA, 543 bp, synthetic construct
  source 1..543: mol_type = other DNA
aggttaatttttaaaaagcagtcaaaagtccaagtggcccttggcagcatttactctctctgtttgctctggttaataatctcaggagcacaaacattccggcccgggaggcgccctttggaccttttgcaatcctggcgcactgaacccttgacccctgccctgcagcccccgcagcttgctgtttgcccactctatttgcccagccccaggggcatataaaacaggggcaaggcacagactcatagcagagtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgaacggtggagggcagtgtagtctgagcagtactcgttgctgccgcgcgcgccaccagacataatagctgacagactaacagactgttcctttccatgggtcttttctgcaggccacc

SEQ ID NO: 150 - DNA, 530 bp, synthetic construct
  source 1..530: mol_type = other DNA
ggcccgggaggcgccctttggaccttttgcaatcctggcgcactgaacccttgacccctgccctgcagcccccgcagcttgctgtttgcccactctatttgcccagccccagccctggagagtcctttagcagggcaaagtgcaacataggcagaccttaagggatgactcagtaacagataagctttgtgtgcctgcagggcatataaaacaggggcaaggcacagactcatagcagagtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgaacggtggagggcagtgtagtctgagcagtactcgttgctgccgcgcgcgccaccagacataatagctgacagactaacagactgttcctttccatgggtcttttctgcaggccacc

SEQ ID NO: 151-153 - skipped (3 entries)

SEQ ID NO: 154 - DNA, 6557 bp, synthetic construct
  source 1..6557: mol_type = other DNA
gccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcctcgagatttaaatgacgttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgagtttaaacttcgtcgaggccgcacgcgtaggttaatttttaaaaagcagtcaaaagtccaagtggcccttggcagcatttactctctctgtttgctctggttaataatctcaggagcacaaacattccccctgttcaaacatgtcctaatactctgtctctgcaagggtcatcagtagttttccatcttactcaacatcctcccagtgagtcatatgtttgctcactgaaggttactagttaacaggcatcccttaaacaggatataaaaggacttcagcaggactgctcgaaacatcccactcagccaccatgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcgtgtccacagccgggcaccgtccccactctacagcgtggagttctccgaggagcccttcggggtgatcgtgcaccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccaatggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcagaggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccaatagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggagaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttagccgggcggagtgtgttagtctctccagagggaggctggttccccagggaagcagagcctgtgtgcgggcagcagctgtgtgcgggcctgggggttgttaagtgcaattatttttaataaaaggggcatttggaaaaaaaaaaaaaaggtagcagtcgacagatgaattctgcagatctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgagttgttgggattccaggcatcgagtagataagtagcatggcgggttaatcattaactacacacgtgcggaccgagcggcctaagctcgcgaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaagacgatttaaatgacaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggc

SEQ ID NO: 155-157 - skipped (3 entries)

SEQ ID NO: 158 - DNA, 7266 bp, synthetic construct
  source 1..7266: mol_type = other DNA
gccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcctcgagatttaaatgacgttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgagtttaaacttcgtcgaggccgcacgcgtaggttaatttttaaaaagcagtcaaaagtccaagtggcccttggcagcatttactctctctgtttgctctggttaataatctcaggagcacaaacattccccctgttcaaacatgtcctaatactctgtctctgcaagggtcatcagtagttttccatcttactcaacatcctcccagtgagtcatatgtttgctcactgaaggttactagttaacaggcatcccttaaacaggatataaaaggacttcagcaggactgctcgaaacatcccacttcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgaacggtggagggcagtgtagtctgagcagtactcgttgctgccgcgcgcgccaccagacataatagctgacagactaacagactgttcctttccatgggtcttttctgcaggccaccatgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcgtgtccacagccgggcaccgtccccactctacagcgtggagttctccgaggagcccttcggggtgatcgtgcaccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccaatggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcagaggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccaatagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggagaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttagcgagcggccgctcttagtagcagtatcgatcccagcccacttttccccaatacgactacgagatctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttggggtccttcaacgtcgaactgtagcactctggatagctagctagcgtttgtagatagtttcaaggatagcaaccaacaacacgatagtttagaaattgttctagatcttctgctactactcgagagatagttgacttctagaagactctggatagagagcttcctgtggtttctatttcaacacttcgagcgatttagatagaggcgtcaattgagaacaaaaactggacacctgtttctagaacactagtgctagtcttctcaaggcttgatacgtcctgccactcacacacaaaaaaccaacacacagattaatgaaaataaagatcttttattagagagcgagtggatagtttagagtcgcttgagacttatcctgtgtccaactagccaagagtagagagagagtgactaggtggatcgagagatcgtcgtctatttgactgctagcctgtgttctcgtcgtagtccaaatcttcaatactctggcgactagtaacttgaatctgtgtatctaacacgacaaggctactcctttgagtttctagtgataagtgcgactggatctagctagttgagtttctagaaactacgtccaaagctactagcactacacgtgcggaccgagcggcctaagctcgcgaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaagacgatttaaatgacaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggc

SEQ ID NO: 159-269 - skipped (111 entries)

SEQ ID NO: 270 - DNA, 280 bp, synthetic construct
  source 1..280: mol_type = other DNA
ccctggagagtcctttagcagggcaaagtgcaacataggcagaccttaagggatgactcagtaacagataagctttgtgtgcctgcagggggatgggaagagggtggggcaggagagggacataaaagggctctgaggcattgtactgtgaattccttcagtctcctgtttggagaagacagagccaatgaggccctcgttccagggaaacagaatatgctcagcatgacgcagcactccctgaactttccggttacatcacccaatagctgagatcaga

SEQ ID NO: 271 - DNA, 241 bp, synthetic construct
  source 1..241: mol_type = other DNA
ccctggagagtcctttagcagggcaaagtgcaacataggcagaccttaagggatgactcagtaacagataagctttgtgtgcctgcagggggatgggaagagggtggggcaggagagggacataaaagggctctgaggcattgtactgtgaattccttcagtctcctgctctgctcagccagtcagccctgcctcccttgtttaggaccacacagcactgctgggtgtctgcctttccttg

SEQ ID NO: 272 - skipped

SEQ ID NO: 273 - DNA, 212 bp, synthetic construct
  source 1..212: mol_type = other DNA
aggttaatttttaaaaagcagtcaaaagtccaagtggcccttggcagcatttactctctctgtttgctctggttaataatctcaggagcacaaacattcctttggagaagacagagccaatgaggccctcgttccagggaaacagaatatgctcagcatgacgcagcactccctgaactttccggttacatcacccaatagctgagatcaga

SEQ ID NO: 274 - DNA, 284 bp, synthetic construct
  source 1..284: mol_type = other DNA
cactgaacccttgacccctgccctgcagcccccgcagcttgctgtttgcccactctatttgcccagccccagaggttaatttttaaaaagcagtcaaaagtccaagtggcccttggcagcatttactctctctgtttgctctggttaataatctcaggagcacaaacattcctttggagaagacagagccaatgaggccctcgttccagggaaacagaatatgctcagcatgacgcagcactccctgaactttccggttacatcacccaatagctgagatcaga

SEQ ID NO: 275 - DNA, 240 bp, synthetic construct
  source 1..240: mol_type = other DNA
cactgaacccttgacccctgccctgcagcccccgcagcttgctgtttgcccactctatttgcccagccccagccctggagagtcctttagcagggcaaagtgcaacataggcagaccttaagggatgactcagtaacagataagctttgtgtgcctgcagggggatgggaagagggtggggcaggagagggacataaaagggctctgaggcattgtactgtgaattccttcagtctcctg

SEQ ID NO: 276 - DNA, 241 bp, synthetic construct; identical to SEQ ID NO: 271
  source 1..241: mol_type = other DNA

SEQ ID NO: 277 - DNA, 168 bp, synthetic construct
  source 1..168: mol_type = other DNA
ccctggagagtcctttagcagggcaaagtgcaacataggcagaccttaagggatgactcagtaacagataagctttgtgtgcctgcagggggatgggaagagggtggggcaggagagggacataaaagggctctgaggcattgtactgtgaattccttcagtctcctg

SEQ ID NO: 278 - DNA, 174 bp, synthetic construct
  source 1..174: mol_type = other DNA
ccctggagagtcctttagcagggcaaagtgcaacataggcagaccttaagggatgactcagtaacagataagctttgtgtgcctgcaccctggagagtcctttagcagggcaaagtgcaacataggcagaccttaagggatgactcagtaacagataagctttgtgtgcctgca

SEQ ID NO: 279 - DNA, 159 bp, synthetic construct
  source 1..159: mol_type = other DNA
cactgaacccttgacccctgccctgcagcccccgcagcttgctgtttgcccactctatttgcccagccccagccctggagagtcctttagcagggcaaagtgcaacataggcagaccttaagggatgactcagtaacagataagctttgtgtgcctgca

SEQ ID NO: 280 - DNA, 207 bp, synthetic construct
  source 1..207: mol_type = other DNA
agaatgaacattgaactttggactatacctgaggggtgaggtaaacaacaggactataaatggcccgggaggcgccctttggaccttttgcaatcctggcgcactgaacccttgacccctgccctgcagcccccgcagcttgctgtttgcccactctatttgcccagccccagggacagagctgatccttgaactcttaagttccac

SEQ ID NO: 281 - DNA, 313 bp, synthetic construct
  source 1..313: mol_type = other DNA
aggttaatttttaaaaagcagtcaaaagtccaagtggcccttggcagcatttactctctctgtttgctctggttaataatctcaggagcacaaacattcctgtaccagaatgaacattgaactttggactatacctgaggggtgaggtaaacaacaggactataaatggcccgggaggcgccctttggaccttttgcaatcctggcgcactgaacccttgacccctgccctgcagcccccgcagcttgctgtttgcccactctatttgcccagccccagggacagagctgatccttgaactcttaagttccac

SEQ ID NO: 282 - DNA, 269 bp, synthetic construct
  source 1..269: mol_type = other DNA
aagcaaatatttgtggttatggattaactcgaactgtttgcccactctatttgccctgtaccagaatgaacattgaactttggactatacctgaggggtgaggtaaacaacaggactataaatggcccgggaggcgccctttggaccttttgcaatcctggcgcactgaacccttgacccctgccctgcagcccccgcagcttgctgtttgcccactctatttgcccagccccagggacagagctgatccttgaactcttaagttccac

SEQ ID NO: 283 - DNA, 258 bp, synthetic construct
  source 1..258: mol_type = other DNA
ggcgccctttggaccttttgcaatcctggagcaaacagcaaacactgtaccagaatgaacattgaactttggactatacctgaggggtgaggtaaacaacaggactataaatggcccgggaggcgccctttggaccttttgcaatcctggcgcactgaacccttgacccctgccctgcagcccccgcagcttgctgtttgcccactctatttgcccagccccagggacagagctgatccttgaactcttaagttccac

SEQ ID NO: 284 - DNA, 194 bp, synthetic construct
  source 1..194: mol_type = other DNA
agaatgaacattgaactttggactatacctgaggggtgaggtaaacaacaggactataaatagaatgaacattgaactttggactatacctgaggggtgaggtaaacaacaggactataaatcactgaacccttgacccctgccctgcagcccccgcagcttgctgtttgcccactctatttgcccagccccag

SEQ ID NO: 285 - DNA, 199 bp, synthetic construct
  source 1..199: mol_type = other DNA
ggcccgggaggcgccctttggaccttttgcaatcctggcgcactgaacccttgacccctgccctgcagcccccgcagcttgctgtttgcccactctatttgcccagccccagccctggagagtcctttagcagggcaaagtgcaacataggcagaccttaagggatgactcagtaacagataagctttgtgtgcctgca

SEQ ID NO: 286 - DNA, 260 bp, synthetic construct
  source 1..260: mol_type = other DNA
agaatgaacattgaactttggactatacctgaggggtgaggtaaacaacaggactataaatggcccgggaggcgccctttggaccttttgcaatcctggcgcactgaacccttgacccctgccctgcagcccccgcagcttgctgtttgcccactctatttgcccagccccagccctggagagtcctttagcagggcaaagtgcaacataggcagaccttaagggatgactcagtaacagataagctttgtgtgcctgca

SEQ ID NO: 287 - DNA, 101 bp, synthetic construct
  source 1..101: mol_type = other DNA
agaatgaacattgaactttggactatacctgaggggtgaggtaaacaacaggactataaatggcccgggaggcgccctttggaccttttgcaatcctggcg

SEQ ID NO: 288 - DNA, 201 bp, synthetic construct
  source 1..201: mol_type = other DNA
gctggtttcttataaaactgatggaagatacaaacactattaaagaactgtttgcatgttgcaaatgatgtccaaagtccaaacattgttaataattaatactccaataaacatcatgtcagaatttctgttttcttttccctttgaacctttgcaggattgccacatcatcaggaccacaccttcatcaggaatgaatat

SEQ ID NO: 289 - DNA, 232 bp, synthetic construct
  source 1..232: mol_type = other DNA
attggcatcttctattgcttttcctggtgacttcatttttcactcttggctaaaaatgggtctctgatgatttattctatcctgggtgttgacaagctgaagaagttgtgtggggcctgctgccagtaaccctgggtgacgaagcgtgactcaccactccgaggtcagtggggggatggaaggcaggggagtcagctgacaagatctgctgctttgtcaccaggccttctgc

SEQ ID NO: 290 - DNA, 200 bp, synthetic construct
  source 1..200: mol_type = other DNA
tgaaatgcctgccatatattagtgccctgaagtccaaaggtagaggaaccgagtgtttaaaaattactgtggctgtggagtcaacatgatgtaaaaaaacaaacatttggataacaccaagaagccagatatggttgaaatgttgactggttgacaaaaataatttgggttgcttaatggtgcacaaaggtaatgcaaaa

SEQ ID NO: 291 - DNA, 103 bp, synthetic construct; identical to SEQ ID NO: 89
  source 1..103: mol_type = other DNA

SEQ ID NO: 292 - DNA, 402 bp, synthetic construct; identical to SEQ ID NO: 87
  source 1..402: mol_type = other DNA

SEQ ID NO: 293 - DNA, 103 bp, synthetic construct; identical to SEQ ID NO: 89
  source 1..103: mol_type = other DNA

SEQ ID NO: 294 - DNA, 299 bp, synthetic construct
  source 1..299: mol_type = other DNA
aggttaatttttaaaaagcagtcaaaagtccaagtggcccttggcagcatttactctctctgtttgctctggttaataatctcaggagcacaaacattccggcccgggaggcgccctttggaccttttgcaatcctggcgcactgaacccttgacccctgccctgcagcccccgcagcttgctgtttgcccactctatttgcccagccccagccctggagagtcctttagcagggcaaagtgcaacataggcagaccttaagggatgactcagtaacagataagctttgtgtgcctgca

SEQ ID NO: 295 - DNA, 212 bp, synthetic construct; identical to SEQ ID NO: 90
  source 1..212: mol_type = other DNA

SEQ ID NO: 296 - DNA, 499 bp, synthetic construct
  source 1..499: mol_type = other DNA
tgaaatgcctgccatatattagtgccctgaagtccaaaggtagaggaaccgagtgtttaaaaattactgtggctgtggagtcaacatgatgtaaaaaaacaaacatttggataacaccaagaagccagatatggttgaaatgttgactggttgacaaaaataatttgggttgcttaatggtgcacaaaggtaatgcaaaaaggttaatttttaaaaagcagtcaaaagtccaagtggcccttggcagcatttactctctctgtttgctctggttaataatctcaggagcacaaacattccggcccgggaggcgccctttggaccttttgcaatcctggcgcactgaacccttgacccctgccctgcagcccccgcagcttgctgtttgcccactctatttgcccagccccagccctggagagtcctttagcagggcaaagtgcaacataggcagaccttaagggatgactcagtaacagataagctttgtgtgcctgca

SEQ ID NO: 297 - DNA, 297 bp, synthetic construct
  source 1..297: mol_type = other DNA
ctttggaccttttgcaatcctggcgcactgaacccttgacccctgccctgcagcccccgcagcttgctgtttgcccactctatttgcccagccccagtgaaatgcctgccatatattagtgccctgaagtccaaaggtagaggaaccgagtgtttaaaaattactgtggctgtggagtcaacatgatgtaaaaaaacaaacatttggataacaccaagaagccagatatggttgaaatgttgactggttgacaaaaataatttgggttgcttaatggtgcacaaaggtaatgcaaaa

SEQ ID NO: 298 - DNA, 212 bp, synthetic construct; identical to SEQ ID NO: 90
  source 1..212: mol_type = other DNA

SEQ ID NO: 299 - DNA, 304 bp, synthetic construct
  source 1..304: mol_type = other DNA
gctggtttcttataaaactgatggaagatacaaacactattaaagaactgtttgcatgttgcaaatgatgtccaaagtccaaacattgttaataattaatactccaataaacatcatgtcagaatttctgttttcttttccctttgaacctttgcaggattgccacatcatcaggaccacaccttcatcaggaatgaatatcaggctttcactttctcgccaacttacaaggcctttctgtgtaaacaatacctgaacctttaccccgttgcccggcaacggccaggtctgtgccaagtgtttg

SEQ ID NO: 300 - DNA, 315 bp, synthetic construct
  source 1..315: mol_type = other DNA
aggttaatttttaaaaagcagtcaaaagtccaagtggcccttggcagcatttactctctctgtttgctctggttaataatctcaggagcacaaacattccggcccgggaggcgccctttggaccttttgcaatcctggcgcactgaacccttgacccctgccctgcagcccccgcagcttgctgtttgcccactctatttgcccagccccagcaggctttcactttctcgccaacttacaaggcctttctgtgtaaacaatacctgaacctttaccccgttgcccggcaacggccaggtctgtgccaagtgtttg

SEQ ID NO: 301 - DNA, 315 bp, synthetic construct; identical to SEQ ID NO: 300
  source 1..315: mol_type = other DNA

SEQ ID NO: 302 - DNA, 413 bp, synthetic construct
  source 1..413: mol_type = other DNA
aggttaatttttaaaaagcagtcaaaagtccaagtggcccttggcagcatttactctctctgtttgctctggttaataatctcaggagcacaaacattccggcccgggaggcgccctttggaccttttgcaatcctggcgcactgaacccttgacccctgccctgcagcccccgcagcttgctgtttgcccactctatttgcccagccccaggctggtttcttataaaactgatggaagatacaaacactattaaagaactgtttgcatgttgcaaatgatgtccaaagtccaaacattgttaataattaatactccaataaacatcatgtcagaatttctgttttcttttccctttgaacctttgcaggattgccacatcatcaggaccacaccttcatcaggaatgaatat

SEQ ID NO: 303 - DNA, 268 bp, synthetic construct
  source 1..268: mol_type = other DNA
aggttaatttttaaaaagcagtcaaaagtccaagtggcccttggcagcatttactctctctgtttgctctggttaataatctcaggagcacaaacattccggcccgggaggcgccctttggaccttttgcaatcctggcgcactgaacccttgacccctgccctgcagcccccgcagcttgctgtttgcccactctatttgcccagccccagaagcaaatatttgtggttatggattaactcgaactgtttgcccactctatttgccc

SEQ ID NO: 304 - DNA, 159 bp, synthetic construct
  source 1..159: mol_type = other DNA
aagcaaatatttgtggttatggattaactcgaactgtttgcccactctatttgccccaggctttcactttctcgccaacttacaaggcctttctgtgtaaacaatacctgaacctttaccccgttgcccggcaacggccaggtctgtgccaagtgtttg

SEQ ID NO: 305 - DNA, 159 bp, synthetic construct; identical to SEQ ID NO: 304
  source 1..159: mol_type = other DNA

SEQ ID NO: 306 - DNA, 243 bp, synthetic construct
  source 1..243: mol_type = other DNA
aggttaatttttaaaaagcagtcaaaagtccaagtggcccttggcagcatttactctctctgtttgctctggttaataatctcaggagcacaaacattccggcccgggaggcgccctttggaccttttgcaatcctggcgcaggctttcactttctcgccaacttacaaggcctttctgtgtaaacaatacctgaacctttaccccgttgcccggcaacggccaggtctgtgccaagtgtttg

SEQ ID NO: 307 - DNA, 243 bp, synthetic construct; identical to SEQ ID NO: 306
  source 1..243: mol_type = other DNA

SEQ ID NO: 308 - DNA, 335 bp, synthetic construct
  source 1..335: mol_type = other DNA
attggcatcttctattgcttttcctggtgacttcatttttcactcttggctaaaaatgggtctctgatgatttattctatcctgggtgttgacaagctgaagaagttgtgtggggcctgctgccagtaaccctgggtgacgaagcgtgactcaccactccgaggtcagtggggggatggaaggcaggggagtcagctgacaagatctgctgctttgtcaccaggccttctgccaggctttcactttctcgccaacttacaaggcctttctgtgtaaacaatacctgaacctttaccccgttgcccggcaacggccaggtctgtgccaagtgtttg

SEQ ID NO: 309 - DNA, 304 bp, synthetic construct
  source 1..304: mol_type = other DNA
caggctttcactttctcgccaacttacaaggcctttctgtgtaaacaatacctgaacctttaccccgttgcccggcaacggccaggtctgtgccaagtgtttggctggtttcttataaaactgatggaagatacaaacactattaaagaactgtttgcatgttgcaaatgatgtccaaagtccaaacattgttaataattaatactccaataaacatcatgtcagaatttctgttttcttttccctttgaacctttgcaggattgccacatcatcaggaccacaccttcatcaggaatgaatat

SEQ ID NO: 310 - DNA, 334 bp, synthetic construct
  source 1..334: mol_type = other DNA
ctgtttgctgcttgcaatgtttgcccattttagggaggttaatttttaaaaagcagtcaaaagtccaagtggcccttggcagcatttactctctctgtttgctctggttaataatctcaggagcacaaacattccggcccgggaggcgccctttggaccttttgcaatcctggcgcactgaacccttgacccctgccctgcagcccccgcagcttgctgtttgcccactctatttgcccagccccagccctggagagtcctttagcagggcaaagtgcaacataggcagaccttaagggatgactcagtaacagataagctttgtgtgcctgca

SEQ ID NO: 311 - DNA, 236 bp, synthetic construct
  source 1..236: mol_type = other DNA
ctgtttgctgcttgcaatgtttgcccattttaggggctggtttcttataaaactgatggaagatacaaacactattaaagaactgtttgcatgttgcaaatgatgtccaaagtccaaacattgttaataattaatactccaataaacatcatgtcagaatttctgttttcttttccctttgaacctttgcaggattgccacatcatcaggaccacaccttcatcaggaatgaatat

SEQ ID NO: 312 - DNA, 103 bp, synthetic construct; identical to SEQ ID NO: 89
  source 1..103: mol_type = other DNA

SEQ ID NO: 313 - DNA, 305 bp, synthetic construct; identical to SEQ ID NO: 88
  source 1..305: mol_type = other DNA

SEQ ID NO: 314 - DNA, 261 bp, synthetic construct
  source 1..261: mol_type = other DNA
aagcaaatatttgtggttatggattaactcgaactgtttgcccactctatttgccctgtaccggcccgggaggcgccctttggaccttttgcaatcctggcgcactgaacccttgacccctgccctgcagcccccgcagcttgctgtttgcccactctatttgcccagccccagccctggagagtcctttagcagggcaaagtgcaacataggcagaccttaagggatgactcagtaacagataagctttgtgtgcctgca

SEQ ID NO: 315 - DNA, 250 bp, synthetic construct
  source 1..250: mol_type = other DNA
ggcgccctttggaccttttgcaatcctggagcaaacagcaaacactgtaccggcccgggaggcgccctttggaccttttgcaatcctggcgcactgaacccttgacccctgccctgcagcccccgcagcttgctgtttgcccactctatttgcccagccccagccctggagagtcctttagcagggcaaagtgcaacataggcagaccttaagggatgactcagtaacagataagctttgtgtgcctgca

SEQ ID NO: 316 - DNA, 366 bp, synthetic construct
  source 1..366: mol_type = other DNA
aggttaatttttaaaaagcagtcaaaagtccaagtggcccttggcagcatttactctctctgtttgctctggttaataatctcaggagcacaaacattcctgtaccagaatgaacattgaactttggactatacctgaggggtgaggtaaacaacaggactataaatggcccgggaggcgccctttggaccttttgcaatcctggcgcactgaacccttgacccctgccctgcagcccccgcagcttgctgtttgcccactctatttgcccagccccagccctggagagtcctttagcagggcaaagtgcaacataggcagaccttaagggatgactcagtaacagataagctttgtgtgcctgca

SEQ ID NO: 317 - DNA, 322 bp, synthetic construct
  source 1..322: mol_type = other DNA
aagcaaatatttgtggttatggattaactcgaactgtttgcccactctatttgccctgtaccagaatgaacattgaactttggactatacctgaggggtgaggtaaacaacaggactataaatggcccgggaggcgccctttggaccttttgcaatcctggcgcactgaacccttgacccctgccctgcagcccccgcagcttgctgtttgcccactctatttgcccagccccagccctggagagtcctttagcagggcaaagtgcaacataggcagaccttaagggatgactcagtaacagataagctttgtgtgcctgca

SEQ ID NO: 318 - DNA, 311 bp, synthetic construct
  source 1..311: mol_type = other DNA
ggcgccctttggaccttttgcaatcctggagcaaacagcaaacactgtaccagaatgaacattgaactttggactatacctgaggggtgaggtaaacaacaggactataaatggcccgggaggcgccctttggaccttttgcaatcctggcgcactgaacccttgacccctgccctgcagcccccgcagcttgctgtttgcccactctatttgcccagccccagccctggagagtcctttagcagggcaaagtgcaacataggcagaccttaagggatgactcagtaacagataagctttgtgtgcctgca

SEQ ID NO: 319 - DNA, 207 bp, synthetic construct
  source 1..207: mol_type = other DNA
aggttaatttttaaaaagcagtcaaaagtccaagtggcccttggcagcatttactctctctgtttgctctggttaataatctcaggagcacaaacattcctgtaccagaatgaacattgaactttggactatacctgaggggtgaggtaaacaacaggactataaatggcccgggaggcgccctttggaccttttgcaatcctggcg

SEQ ID NO: 320 - DNA, 163 bp, synthetic construct
  source 1..163: mol_type = other DNA
aagcaaatatttgtggttatggattaactcgaactgtttgcccactctatttgccctgtaccagaatgaacattgaactttggactatacctgaggggtgaggtaaacaacaggactataaatggcccgggaggcgccctttggaccttttgcaatcctggcg

SEQ ID NO: 321 - DNA, 152 bp, synthetic construct
  source 1..152: mol_type = other DNA
ggcgccctttggaccttttgcaatcctggagcaaacagcaaacactgtaccagaatgaacattgaactttggactatacctgaggggtgaggtaaacaacaggactataaatggcccgggaggcgccctttggaccttttgcaatcctggcg

SEQ ID NO: 322 - DNA, 100 bp, synthetic construct
  source 1..100: mol_type = other DNA
aggttaatttttaaaaagcagtcaaaagtccaagtggcccttggcagcatttactctctctgtttgctctggttaataatctcaggagcacaaacattcc

SEQ ID NO: 323 - DNA, 146 bp, synthetic construct
  source 1..146: mol_type = other DNA
aggttaatttttaaaaagcagtcaaaagtccaagtggcccttggcagcatttactctctctgtttgctctggttaataatctcaggagcacaaacattcctgtaccggcccgggaggcgccctttggaccttttgcaatcctggcg

SEQ ID NO: 324 - DNA, 172 bp, synthetic construct
  source 1..172: mol_type = other DNA
tgctctctgacaaagatacggtgggtcccactgatgaactgtgctgccacagtaaatgtagccactatgcctatctccattctgaagatgtgccctgttcaaacatgtcctaatactctgtctctgcaagggtcatcagtagttttccatcttactcaacatcctcccagtg

SEQ ID NO: 325 - DNA, 180 bp, synthetic construct
  source 1..180: mol_type = other DNA
cctccccgtgttcctgctctttgtccctctgtcctacttagactaatatttgccttgggtactgcaaacaggaaatgggggagggacaggagtagggcggccctgttcaaacatgtcctaatactctgtctctgcaagggtcatcagtagttttccatcttactcaacatcctcccagtg

SEQ ID NO: 326 - DNA, 159 bp, synthetic construct
  source 1..159: mol_type = other DNA
ccgcccccactgaacccttgacccctgccctgcagcccccgcagcttgctgtttgcccactctatttgcccagccccagccctgttcaaacatgtcctaatactctgtctctgcaagggtcatcagtagttttccatcttactcaacatcctcccagtg

SEQ ID NO: 327 - DNA, 165 bp, synthetic construct
  source 1..165: mol_type = other DNA
aagctttctgaacagccaaacagagattccaaagttcaggcaccaaagttcagaccctaacagttatttacaagggtcagttaacccctgttcaaacatgtcctaatactctgtctctgcaagggtcatcagtagttttccatcttactcaacatcctcccagtg

SEQ ID NO: 328 - DNA, 140 bp, synthetic construct
  source 1..140: mol_type = other DNA
aggttaatttttaaaaagcagtcaaaagtccaagtggcccttggcagcatttactctctctgtttgctctggttaataatctcaggagcacaaacattccggcccgggaggcgccctttggaccttttgcaatcctggcg

SEQ ID NO: 329 - DNA, 100 bp, synthetic construct; identical to SEQ ID NO: 322
  source 1..100: mol_type = other DNA

SEQ ID NO: 330 - DNA, 233 bp, synthetic construct
  source 1..233: mol_type = other DNA
aggttaatttttaaaaagcagtcaaaagtccaagtggcccttggcagcatttactctctctgtttgctctggttaataatctcaggagcacaaacattcctgtaccggcccgggaggcgccctttggaccttttgcaatcctggcgccctggagagtcctttagcagggcaaagtgcaacataggcagaccttaagggatgactcagtaacagataagctttgtgtgcctgca

SEQ ID NO: 331 - DNA, 265 bp, synthetic construct
  source 1..265: mol_type = other DNA
aggttaatttttaaaaagcagtcaaaagtccaagtggcccttggcagcatttactctctctgtttgctctggttaataatctcaggagcacaaacattcctgtacccactgaacccttgacccctgccctgcagcccccgcagcttgctgtttgcccactctatttgcccagccccagccctggagagtcctttagcagggcaaagtgcaacataggcagaccttaagggatgactcagtaacagataagctttgtgtgcctgca

SEQ ID NO: 332 - DNA, 469 bp, synthetic construct
  source 1..469: mol_type = other DNA
caggctttcactttctcgccaacttacaaggcctttctgtgtaaacaatacctgaacctttaccccgttgcccggcaacggccaggtctgtgccaagtgtttgaggttaatttttaaaaagcagtcaaaagtccaagtggcccttggcagcatttactctctctgtttgctctggttaataatctcaggagcacaaacattcctgtaccagaatgaacattgaactttggactatacctgaggggtgaggtaaacaacaggactataaatggcccgggaggcgccctttggaccttttgcaatcctggcgcactgaacccttgacccctgccctgcagcccccgcagcttgctgtttgcccactctatttgcccagccccagccctggagagtcctttagcagggcaaagtgcaacataggcagaccttaagggatgactcagtaacagataagctttgtgtgcctgca

SEQ ID NO: 333 - DNA, 61 bp, synthetic construct
  source 1..61: mol_type = other DNA
agaatgaacattgaactttggactatacctgaggggtgaggtaaacaacaggactataaat

SEQ ID NO: 334 - DNA, 364 bp, synthetic construct
  source 1..364: mol_type = other DNA
caggctttcactttctcgccaacttacaaggcctttctgtgtaaacaatacctgaacctttaccccgttgcccggcaacggccaggtctgtgccaagtgtttgaagcaaatatttgtggttatggattaactcgaactgtttgcccactctatttgccctgtaccggcccgggaggcgccctttggaccttttgcaatcctggcgcactgaacccttgacccctgccctgcagcccccgcagcttgctgtttgcccactctatttgcccagccccagccctggagagtcctttagcagggcaaagtgcaacataggcagaccttaagggatgactcagtaacagataagctttgtgtgcctgca

SEQ ID NO: 335 - DNA, 425 bp, synthetic construct
  source 1..425: mol_type = other DNA
caggctttcactttctcgccaacttacaaggcctttctgtgtaaacaatacctgaacctttaccccgttgcccggcaacggccaggtctgtgccaagtgtttgaagcaaatatttgtggttatggattaactcgaactgtttgcccactctatttgccctgtaccagaatgaacattgaactttggactatacctgaggggtgaggtaaacaacaggactataaatggcccgggaggcgccctttggaccttttgcaatcctggcgcactgaacccttgacccctgccctgcagcccccgcagcttgctgtttgcccactctatttgcccagccccagccctggagagtcctttagcagggcaaagtgcaacataggcagaccttaagggatgactcagtaacagataagctttgtgtgcctgca

SEQ ID NO: 336 - DNA, 338 bp, synthetic construct
  source 1..338: mol_type = other DNA
caggctttcactttctcgccaacttacaaggcctttctgtgtaaacaatacctgaacctttaccccgttgcccggcaacggccaggtctgtgccaagtgtttgaagcaaatatttgtggttatggattaactcgaactgtttgcccactctatttgccctgtaccagaatgaacattgaactttggactatacctgaggggtgaggtaaacaacaggactataaatggcccgggaggcgccctttggaccttttgcaatcctggcgcactgaacccttgacccctgccctgcagcccccgcagcttgctgtttgcccactctatttgcccagccccag

SEQ ID NO: 337 - DNA, 140 bp, synthetic construct; identical to SEQ ID NO: 328
  source 1..140: mol_type = other DNA

SEQ ID NO: 338 - DNA, 203 bp, synthetic construct
  source 1..203: mol_type = other DNA
aggttaatttttaaaaagcagtcaaaagtccaagtggcccttggcagcatttactctctctgtttgctctggttaataatctcaggagcacaaacattcccaggctttcactttctcgccaacttacaaggcctttctgtgtaaacaatacctgaacctttaccccgttgcccggcaacggccaggtctgtgccaagtgtttg

SEQ ID NO: 339 - DNA, 180 bp, synthetic construct; identical to SEQ ID NO: 86
  source 1..180: mol_type = other DNA

SEQ ID NO: 340 - DNA, 80 bp, synthetic construct
  source 1..80: mol_type = other DNA
ccctgttcaaacatgtcctaatactctgtctctgcaagggtcatcagtagttttccatcttactcaacatcctcccagtg

SEQ ID NO: 341 - DNA, 146 bp, synthetic construct; identical to SEQ ID NO: 323
  source 1..146: mol_type = other DNA

SEQ ID NO: 342 - DNA, 356 bp, synthetic construct
  source 1..356: mol_type = other DNA
ccctggagagtcctttagcagggcaaagtgcaacataggcagaccttaagggatgactcagtaacagataagctttgtgtgcctgcagggggatgggaagagggtggggcaggagagggacataaaagggctctgaggcattgtactgtgaattccttcagtctcctgtttggagaagacagagccaatgaggccctcgttccagggaaacagaatatgctcagcatgacgcagcactccctgaactttccggttacatcacccaatagctgagatcagagggcatataaaacaggggcaaggcacagactcatagcagagcaatcaccaccaagcctggaataactgcagccacc

SEQ ID NO: 343 - DNA, 453 bp, synthetic construct
  source 1..453: mol_type = other DNA
cctggagagtcctttagcagggcaaagtgcaacataggcagaccttaagggatgactcagtaacagataagctttgtgtgcctgcagggggatgggaagagggtggggcaggagagggacataaaagggctctgaggcattgtactgtgaattccttcagtctcctgctctgctcagccagtcagccctgcctcccttgtttaggaccacacagcactgctgggtgtctgcctttccttgggtaatttttttttctggttaatatttagcaagaattctgcagagtgatcaaaaaaatcaaatactcagtatttcagaaatagattaaataggttacttttttactgataatgtgaaagaatgatataaaaacttgattttcctcaacaacattactttcttttgtaaatgtggtttctacaaagatgaaactactaaaacttacaggccacc

SEQ ID NO: 344 - DNA, 288 bp, synthetic construct
  source 1..288: mol_type = other DNA
aggttaatttttaaaaagcagtcaaaagtccaagtggcccttggcagcatttactctctctgtttgctctggttaataatctcaggagcacaaacattcctttggagaagacagagccaatgaggccctcgttccagggaaacagaatatgctcagcatgacgcagcactccctgaactttccggttacatcacccaatagctgagatcagagggcatataaaacaggggcaaggcacagactcatagcagagcaatcaccaccaagcctggaataactgcagccacc

SEQ ID NO: 345 - DNA, 360 bp, synthetic construct
  source 1..360: mol_type = other DNA
cactgaacccttgacccctgccctgcagcccccgcagcttgctgtttgcccactctatttgcccagccccagaggttaatttttaaaaagcagtcaaaagtccaagtggcccttggcagcatttactctctctgtttgctctggttaataatctcaggagcacaaacattcctttggagaagacagagccaatgaggccctcgttccagggaaacagaatatgctcagcatgacgcagcactccctgaactttccggttacatcacccaatagctgagatcagagggcatataaaacaggggcaaggcacagactcatagcagagcaatcaccaccaagcctggaataactgcagccacc

SEQ ID NO: 346 - DNA, 316 bp, synthetic construct
  source 1..316: mol_type = other DNA
cactgaacccttgacccctgccctgcagcccccgcagcttgctgtttgcccactctatttgcccagccccagccctggagagtcctttagcagggcaaagtgcaacataggcagaccttaagggatgactcagtaacagataagctttgtgtgcctgcagggggatgggaagagggtggggcaggagagggacataaaagggctctgaggcattgtactgtgaattccttcagtctcctggggcatataaaacaggggcaaggcacagactcatagcagagcaatcaccaccaagcctggaataactgcagccacc

SEQ ID NO: 347 - DNA, 411 bp, synthetic construct
  source 1..411: mol_type = other DNA
ccctggagagtcctttagcagggcaaagtgcaacataggcagaccttaagggatgactcagtaacagataagctttgtgtgcctgcagggggatgggaagagggtggggcaggagagggacataaaagggctctgaggcattgtactgtgaattccttcagtctcctgctctgctcagccagtcagccctgcctcccttgtttaggaccacacagcactgctgggtgtctgcctttccttgtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgccacc

SEQ ID NO: 348 - DNA, 338 bp, synthetic construct
  source 1..338: mol_type = other DNA
ccctggagagtcctttagcagggcaaagtgcaacataggcagaccttaagggatgactcagtaacagataagctttgtgtgcctgcagggggatgggaagagggtggggcaggagagggacataaaagggctctgaggcattgtactgtgaattccttcagtctcctgtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgccacc

SEQ ID NO: 349 - DNA, 250 bp, synthetic construct
  source 1..250: mol_type = other DNA
ccctggagagtcctttagcagggcaaagtgcaacataggcagaccttaagggatgactcagtaacagataagctttgtgtgcctgcaccctggagagtcctttagcagggcaaagtgcaacataggcagaccttaagggatgactcagtaacagataagctttgtgtgcctgcagggcatataaaacaggggcaaggcacagactcatagcagagcaatcaccaccaagcctggaataactgcagccacc

SEQ ID NO: 350 - DNA, 385 bp, synthetic construct
  source 1..385: mol_type = other DNA
cactgaacccttgacccctgccctgcagcccccgcagcttgctgtttgcccactctatttgcccagccccagccctggagagtcctttagcagggcaaagtgcaacataggcagaccttaagggatgactcagtaacagataagctttgtgtgcctgcactcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagatgcgccacc

SEQ ID NO: 351 - DNA, 283 bp, synthetic construct
  source 1..283: mol_type = other DNA
agaatgaacattgaactttggactatacctgaggggtgaggtaaacaacaggactataaatggcccgggaggcgccctttggaccttttgcaatcctggcgcactgaacccttgacccctgccctgcagcccccgcagcttgctgtttgcccactctatttgcccagccccagggacagagctgatccttgaactcttaagttccacgggcatataaaacaggggcaaggcacagactcatagcagagcaatcaccaccaagcctggaataactgcagccacc

SEQ ID NO: 352 - DNA, 389 bp, synthetic construct
  source 1..389: mol_type = other DNA
aggttaatttttaaaaagcagtcaaaagtccaagtggcccttggcagcatttactctctctgtttgctctggttaataatctcaggagcacaaacattcctgtaccagaatgaacattgaactttggactatacctgaggggtgaggtaaacaacaggactataaatggcccgggaggcgccctttggaccttttgcaatcctggcgcactgaacccttgacccctgccctgcagcccccgcagcttgctgtttgcccactctatttgcccagccccagggacagagctgatccttgaactcttaagttccacgggcatataaaacaggggcaaggcacagactcatagcagagcaatcaccaccaagcctggaataactgcagccacc

SEQ ID NO: 353 - DNA, 345 bp, synthetic construct
  source 1..345: mol_type = other DNA
aagcaaatatttgtggttatggattaactcgaactgtttgcccactctatttgccctgtaccagaatgaacattgaactttggactatacctgaggggtgaggtaaacaacaggactataaatggcccgggaggcgccctttggaccttttgcaatcctggcgcactgaacccttgacccctgccctgcagcccccgcagcttgctgtttgcccactctatttgcccagccccagggacagagctgatccttgaactcttaagttccacgggcatataaaacaggggcaaggcacagactcatagcagagcaatcaccaccaagcctggaataactgcagccacc

SEQ ID NO: 354 - DNA, 334 bp, synthetic construct
  source 1..334: mol_type = other DNA
ggcgccctttggaccttttgcaatcctggagcaaacagcaaacactgtaccagaatgaacattgaactttggactatacctgaggggtgaggtaaacaacaggactataaatggcccgggaggcgccctttggaccttttgcaatcctggcgcactgaacccttgacccctgccctgcagcccccgcagcttgctgtttgcccactctatttgcccagccccagggacagagctgatccttgaactcttaagttccacgggcatataaaacaggggcaaggcacagactcatagcagagcaatcaccaccaagcctggaataactgcagccacc

SEQ ID NO: 355 - DNA, 270 bp, synthetic construct
  source 1..270: mol_type = other DNA
agaatgaacattgaactttggactatacctgaggggtgaggtaaacaacaggactataaatagaatgaacattgaactttggactatacctgaggggtgaggtaaacaacaggactataaatcactgaacccttgacccctgccctgcagcccccgcagcttgctgtttgcccactctatttgcccagccccaggggcatataaaacaggggcaaggcacagactcatagcagagcaatcaccaccaagcctggaataactgcagccacc

SEQ ID NO: 356 - DNA, 275 bp, synthetic construct; identical to SEQ ID NO: 144
  source 1..275: mol_type = other DNA

SEQ ID NO: 357 - DNA, 336 bp, synthetic construct
  source 1..336: mol_type = other DNA
agaatgaacattgaactttggactatacctgaggggtgaggtaaacaacaggactataaatggcccgggaggcgccctttggaccttttgcaatcctggcgcactgaacccttgacccctgccctgcagcccccgcagcttgctgtttgcccactctatttgcccagccccagccctggagagtcctttagcagggcaaagtgcaacataggcagaccttaagggatgactcagtaacagataagctttgtgtgcctgcagggcatataaaacaggggcaaggcacagactcatagcagagcaatcaccaccaagcctggaataactgcagccacc

SEQ ID NO: 358 - DNA, 327 bp, synthetic construct
  source 1..327: mol_type = other DNA
agaatgaacattgaactttggactatacctgaggggtgaggtaaacaacaggactataaatggcccgggaggcgccctttggaccttttgcaatcctggcgctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagatgcgccacc

SEQ ID NO: 359 - DNA, 480 bp, synthetic construct
  source 1..480: mol_type = other DNA
gctggtttcttataaaactgatggaagatacaaacactattaaagaactgtttgcatgttgcaaatgatgtccaaagtccaaacattgttaataattaatactccaataaacatcatgtcagaatttctgttttcttttccctttgaacctttgcaggattgccacatcatcaggaccacaccttcatcaggaatgaatatccgatgacctaatgattctgagcttggcaaaggtcttatctcccagctcgcccaggcccagtgttccaggaatgtgacctttgctgcagcagccgctggagggggcagaggggatgggctggaggttgagcaaacagagcagcagaaaaggcagttcctcttctccagtgccctccttccctgtctctgcctctccctcccttcctcaggcatcagagcggagacttcagggagaccagagcccagcttgccaggcactgagctagaagccctgccatg

SEQ ID NO: 360 - DNA, 511 bp, synthetic construct
  source 1..511: mol_type = other DNA
attggcatcttctattgcttttcctggtgacttcatttttcactcttggctaaaaatgggtctctgatgatttattctatcctgggtgttgacaagctgaagaagttgtgtggggcctgctgccagtaaccctgggtgacgaagcgtgactcaccactccgaggtcagtggggggatggaaggcaggggagtcagctgacaagatctgctgctttgtcaccaggccttctgcccgatgacctaatgattctgagcttggcaaaggtcttatctcccagctcgcccaggcccagtgttccaggaatgtgacctttgctgcagcagccgctggagggggcagaggggatgggctggaggttgagcaaacagagcagcagaaaaggcagttcctcttctccagtgccctccttccctgtctctgcctctccctcccttcctcaggcatcagagcggagacttcagggagaccagagcccagcttgccaggcactgagctagaagccctgccatg

SEQ ID NO: 361 - DNA, 479 bp, synthetic construct
  source 1..479: mol_type = other DNA
tgaaatgcctgccatatattagtgccctgaagtccaaaggtagaggaaccgagtgtttaaaaattactgtggctgtggagtcaacatgatgtaaaaaaacaaacatttggataacaccaagaagccagatatggttgaaatgttgactggttgacaaaaataatttgggttgcttaatggtgcacaaaggtaatgcaaaaccgatgacctaatgattctgagcttggcaaaggtcttatctcccagctcgcccaggcccagtgttccaggaatgtgacctttgctgcagcagccgctggagggggcagaggggatgggctggaggttgagcaaacagagcagcagaaaaggcagttcctcttctccagtgccctccttccctgtctctgcctctccctcccttcctcaggcatcagagcggagacttcagggagaccagagcccagcttgccaggcactgagctagaagccctgccatg

SEQ ID NO: 362 - DNA, 340 bp, synthetic construct
  source 1..340: mol_type = other DNA
caggctttcactttctcgccaacttacaaggcctttctgtgtaaacaatacctgaacctttaccccgttgcccggcaacggccaggtctgtgccaagtgtttgcctttgcaacagcttatcggaagcaaacaagctgaggggaattgagcaagaatttctgggataccaacagcataggaggaacaaaggacgtagagggagggttgactgtctacacaggacaaagccaatgattaaccaaacctcttgcagatttaaataggatgggaactaggagtggcagcaatcctttctttcagctggagtgctcctcaggagccagccccacccttagaaaag

SEQ ID NO: 363 - DNA, 575 bp, synthetic construct
  source 1..575: mol_type = other DNA
aggttaatttttaaaaagcagtcaaaagtccaagtggcccttggcagcatttactctctctgtttgctctggttaataatctcaggagcacaaacattccggcccgggaggcgccctttggaccttttgcaatcctggcgcactgaacccttgacccctgccctgcagcccccgcagcttgctgtttgcccactctatttgcccagccccagccctggagagtcctttagcagggcaaagtgcaacataggcagaccttaagggatgactcagtaacagataagctttgtgtgcctgcaccgatgacctaatgattctgagcttggcaaaggtcttatctcccagctcgcccaggcccagtgttccaggaatgtgacctttgctgcagcagccgctggagggggcagaggggatgggctggaggttgagcaaacagagcagcagaaaaggcagttcctcttctccagtgccctccttccctgtctctgcctctccctcccttcctcaggcatcagagcggagacttcagggagaccagagcccagcttgccaggcactgagctagaagccctgcc

SEQ ID NO: 364 - DNA, 488 bp, synthetic construct
  source 1..488: mol_type = other DNA
aggttaatttttaaaaagcagtcaaaagtccaagtggcccttggcagcatttactctctctgtttgctctggttaataatctcaggagcacaaacattccggcccgggaggcgccctttggaccttttgcaatcctggcgcactgaacccttgacccctgccctgcagcccccgcagcttgctgtttgcccactctatttgcccagccccagccgatgacctaatgattctgagcttggcaaaggtcttatctcccagctcgcccaggcccagtgttccaggaatgtgacctttgctgcagcagccgctggagggggcagaggggatgggctggaggttgagcaaacagagcagcagaaaaggcagttcctcttctccagtgccctccttccctgtctctgcctctccctcccttcctcaggcatcagagcggagacttcagggagaccagagcccagcttgccaggcactgagctagaagccctgcc

SEQ ID NO: 365 - DNA, 575 bp, synthetic construct
  source 1..575: mol_type = other DNA
tgaaatgcctgccatatattagtgccctgaagtccaaaggtagaggaaccgagtgtttaaaaattactgtggctgtggagtcaacatgatgtaaaaaaacaaacatttggataacaccaagaagccagatatggttgaaatgttgactggttgacaaaaataatttgggttgcttaatggtgcacaaaggtaatgcaaaaaggttaatttttaaaaagcagtcaaaagtccaagtggcccttggcagcatttactctctctgtttgctctggttaataatctcaggagcacaaacattccggcccgggaggcgccctttggaccttttgcaatcctggcgcactgaacccttgacccctgccctgcagcccccgcagcttgctgtttgcccactctatttgcccagccccagccctggagagtcctttagcagggcaaagtgcaacataggcagaccttaagggatgactcagtaacagataagctttgtgtgcctgcagggcatataaaacaggggcaaggcacagactcatagcagagcaatcaccaccaagcctggaataactgcagccacc

SEQ ID NO: 366 - DNA, 688 bp, synthetic construct
  source 1..688: mol_type = other DNA
aggttaatttttaaaaagcagtcaaaagtccaagtggcccttggcagcatttactctctctgtttgctctggttaataatctcaggagcacaaacattccggcccgggaggcgccctttggaccttttgcaatcctggcgcactgaacccttgacccctgccctgcagcccccgcagcttgctgtttgcccactctatttgcccagccccagtgaaatgcctgccatatattagtgccctgaagtccaaaggtagaggaaccgagtgtttaaaaattactgtggctgtggagtcaacatgatgtaaaaaaacaaacatttggataacaccaagaagccagatatggttgaaatgttgactggttgacaaaaataatttgggttgcttaatggtgcacaaaggtaatgcaaaaccgatgacctaatgattctgagcttggcaaaggtcttatctcccagctcgcccaggcccagtgttccaggaatgtgacctttgctgcagcagccgctggagggggcagaggggatgggctggaggttgagcaaacagagcagcagaaaaggcagttcctcttctccagtgccctccttccctgtctctgcctctccctcccttcctcaggcatcagagcggagacttcagggagaccagagcccagcttgccaggcactgagctagaagccctgcc

SEQ ID NO: 367 - DNA, 580 bp, synthetic construct
  source 1..580: mol_type = other DNA
gctggtttcttataaaactgatggaagatacaaacactattaaagaactgtttgcatgttgcaaatgatgtccaaagtccaaacattgttaataattaatactccaataaacatcatgtcagaatttctgttttcttttccctttgaacctttgcaggattgccacatcatcaggaccacaccttcatcaggaatgaatatcaggctttcactttctcgccaacttacaaggcctttctgtgtaaacaatacctgaacctttaccccgttgcccggcaacggccaggtctgtgccaagtgtttgccgatgacctaatgattctgagcttggcaaaggtcttatctcccagctcgcccaggcccagtgttccaggaatgtgacctttgctgcagcagccgctggagggggcagaggggatgggctggaggttgagcaaacagagcagcagaaaaggcagttcctcttctccagtgccctccttccctgtctctgcctctccctcccttcctcaggcatcagagcggagacttcagggagaccagagcccagcttgccaggcactgagctagaagccctgcc

SEQ ID NO: 368 - DNA, 391 bp, synthetic construct
  source 1..391: mol_type = other DNA
aggttaatttttaaaaagcagtcaaaagtccaagtggcccttggcagcatttactctctctgtttgctctggttaataatctcaggagcacaaacattccggcccgggaggcgccctttggaccttttgcaatcctggcgcactgaacccttgacccctgccctgcagcccccgcagcttgctgtttgcccactctatttgcccagccccagcaggctttcactttctcgccaacttacaaggcctttctgtgtaaacaatacctgaacctttaccccgttgcccggcaacggccaggtctgtgccaagtgtttggggcatataaaacaggggcaaggcacagactcatagcagagcaatcaccaccaagcctggaataactgcagccacc

SEQ ID NO: 369 - DNA, 507 bp, synthetic construct
  source 1..507: mol_type = other DNA
aggttaatttttaaaaagcagtcaaaagtccaagtggcccttggcagcatttactctctctgtttgctctggttaataatctcaggagcacaaacattccggcccgggaggcgccctttggaccttttgcaatcctggcgcactgaacccttgacccctgccctgcagcccccgcagcttgctgtttgcccactctatttgcccagccccagcaggctttcactttctcgccaacttacaaggcctttctgtgtaaacaatacctgaacctttaccccgttgcccggcaacggccaggtctgtgccaagtgtttggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgccacc

SEQ ID NO: 370 - DNA, 689 bp, synthetic construct
  source 1..689: mol_type = other DNA
aggttaatttttaaaaagcagtcaaaagtccaagtggcccttggcagcatttactctctctgtttgctctggttaataatctcaggagcacaaacattccggcccgggaggcgccctttggaccttttgcaatcctggcgcactgaacccttgacccctgccctgcagcccccgcagcttgctgtttgcccactctatttgcccagccccaggctggtttcttataaaactgatggaagatacaaacactattaaagaactgtttgcatgttgcaaatgatgtccaaagtccaaacattgttaataattaatactccaataaacatcatgtcagaatttctgttttcttttccctttgaacctttgcaggattgccacatcatcaggaccacaccttcatcaggaatgaatatccgatgacctaatgattctgagcttggcaaaggtcttatctcccagctcgcccaggcccagtgttccaggaatgtgacctttgctgcagcagccgctggagggggcagaggggatgggctggaggttgagcaaacagagcagcagaaaaggcagttcctcttctccagtgccctccttccctgtctctgcctctccctcccttcctcaggcatcagagcggagacttcagggagaccagagcccagcttgccaggcactgagctagaagccctgcc

SEQ ID NO: 371 - DNA, 344 bp, synthetic construct
  source 1..344: mol_type = other DNA
aggttaatttttaaaaagcagtcaaaagtccaagtggcccttggcagcatttactctctctgtttgctctggttaataatctcaggagcacaaacattccggcccgggaggcgccctttggaccttttgcaatcctggcgcactgaacccttgacccctgccctgcagcccccgcagcttgctgtttgcccactctatttgcccagccccagaagcaaatatttgtggttatggattaactcgaactgtttgcccactctatttgcccgggcatataaaacaggggcaaggcacagactcatagcagagcaatcaccaccaagcctggaataactgcagccacc

SEQ ID NO: 372 - DNA, 401 bp, synthetic construct
  source 1..401: mol_type = other DNA
aagcaaatatttgtggttatggattaactcgaactgtttgcccactctatttgccccaggctttcactttctcgccaacttacaaggcctttctgtgtaaacaatacctgaacctttaccccgttgcccggcaacggccaggtctgtgccaagtgtttgcctttgcaacagcttatcggaagcaaacaagctgaggggaattgagcaagaatttctgggataccaacagcataggaggaacaaaggacgtagagggagggttgactgtctacacaggacaaagccaatgattaaccaaacctcttgcagatttaaataggatgggaactaggagtggcagcaatcctttctttcagctggagtgctcctcaggagccagccccacccttagaaaagccacc

SEQ ID NO: 373 - DNA, 435 bp, synthetic construct
  source 1..435: mol_type = other DNA
aagcaaatatttgtggttatggattaactcgaactgtttgcccactctatttgccccaggctttcactttctcgccaacttacaaggcctttctgtgtaaacaatacctgaacctttaccccgttgcccggcaacggccaggtctgtgccaagtgtttgccgatgacctaatgattctgagcttggcaaaggtcttatctcccagctcgcccaggcccagtgttccaggaatgtgacctttgctgcagcagccgctggagggggcagaggggatgggctggaggttgagcaaacagagcagcagaaaaggcagttcctcttctccagtgccctccttccctgtctctgcctctccctcccttcctcaggcatcagagcggagacttcagggagaccagagcccagcttgccaggcactgagctagaagccctgcc

SEQ ID NO: 374 - DNA, 485 bp, synthetic construct
  source 1..485: mol_type = other DNA
aggttaatttttaaaaagcagtcaaaagtccaagtggcccttggcagcatttactctctctgtttgctctggttaataatctcaggagcacaaacattccggcccgggaggcgccctttggaccttttgcaatcctggcgcaggctttcactttctcgccaacttacaaggcctttctgtgtaaacaatacctgaacctttaccccgttgcccggcaacggccaggtctgtgccaagtgtttgcctttgcaacagcttatcggaagcaaacaagctgaggggaattgagcaagaatttctgggataccaacagcataggaggaacaaaggacgtagagggagggttgactgtctacacaggacaaagccaatgattaaccaaacctcttgcagatttaaataggatgggaactaggagtggcagcaatcctttctttcagctggagtgctcctcaggagccagccccacccttagaaaagccacc

SEQ ID NO: 375 - DNA, 519 bp, synthetic construct
  source 1..519: mol_type = other DNA
aggttaatttttaaaaagcagtcaaaagtccaagtggcccttggcagcatttactctctctgtttgctctggttaataatctcaggagcacaaacattccggcccgggaggcgccctttggaccttttgcaatcctggcgcaggctttcactttctcgccaacttacaaggcctttctgtgtaaacaatacctgaacctttaccccgttgcccggcaacggccaggtctgtgccaagtgtttgccgatgacctaatgattctgagcttggcaaaggtcttatctcccagctcgcccaggcccagtgttccaggaatgtgacctttgctgcagcagccgctggagggggcagaggggatgggctggaggttgagcaaacagagcagcagaaaaggcagttcctcttctccagtgccctccttccctgtctctgcctctccctcccttcctcaggcatcagagcggagacttcagggagaccagagcccagcttgccaggcactgagctagaagccctgcc

SEQ ID NO: 376 - DNA, 611 bp, synthetic construct
  source 1..611: mol_type = other DNA
attggcatcttctattgcttttcctggtgacttcatttttcactcttggctaaaaatgggtctctgatgatttattctatcctgggtgttgacaagctgaagaagttgtgtggggcctgctgccagtaaccctgggtgacgaagcgtgactcaccactccgaggtcagtggggggatggaaggcaggggagtcagctgacaagatctgctgctttgtcaccaggccttctgccaggctttcactttctcgccaacttacaaggcctttctgtgtaaacaatacctgaacctttaccccgttgcccggcaacggccaggtctgtgccaagtgtttgccgatgacctaatgattctgagcttggcaaaggtcttatctcccagctcgcccaggcccagtgttccaggaatgtgacctttgctgcagcagccgctggagggggcagaggggatgggctggaggttgagcaaacagagcagcagaaaaggcagttcctcttctccagtgccctccttccctgtctctgcctctccctcccttcctcaggcatcagagcggagacttcagggagaccagagcccagcttgccaggcactgagctagaagccctgcc

SEQ ID NO: 377 - DNA, 580 bp, synthetic construct
  source 1..580: mol_type = other DNA
caggctttcactttctcgccaacttacaaggcctttctgtgtaaacaatacctgaacctttaccccgttgcccggcaacggccaggtctgtgccaagtgtttggctggtttcttataaaactgatggaagatacaaacactattaaagaactgtttgcatgttgcaaatgatgtccaaagtccaaacattgttaataattaatactccaataaacatcatgtcagaatttctgttttcttttccctttgaacctttgcaggattgccacatcatcaggaccacaccttcatcaggaatgaatatccgatgacctaatgattctgagcttggcaaaggtcttatctcccagctcgcccaggcccagtgttccaggaatgtgacctttgctgcagcagccgctggagggggcagaggggatgggctggaggttgagcaaacagagcagcagaaaaggcagttcctcttctccagtgccctccttccctgtctctgcctctccctcccttcctcaggcatcagagcggagacttcagggagaccagagcccagcttgccaggcactgagctagaagccctgcc

SEQ ID NO: 378 - DNA, 410 bp, synthetic construct
  source 1..410: mol_type = other DNA
ctgtttgctgcttgcaatgtttgcccattttagggaggttaatttttaaaaagcagtcaaaagtccaagtggcccttggcagcatttactctctctgtttgctctggttaataatctcaggagcacaaacattccggcccgggaggcgccctttggaccttttgcaatcctggcgcactgaacccttgacccctgccctgcagcccccgcagcttgctgtttgcccactctatttgcccagccccagccctggagagtcctttagcagggcaaagtgcaacataggcagaccttaagggatgactcagtaacagataagctttgtgtgcctgcagggcatataaaacaggggcaaggcacagactcatagcagagcaatcaccaccaagcctggaataactgcagccacc

SEQ ID NO: 379 - DNA, 512 bp, synthetic construct
  source 1..512: mol_type = other DNA
ctgtttgctgcttgcaatgtttgcccattttaggggctggtttcttataaaactgatggaagatacaaacactattaaagaactgtttgcatgttgcaaatgatgtccaaagtccaaacattgttaataattaatactccaataaacatcatgtcagaatttctgttttcttttccctttgaacctttgcaggattgccacatcatcaggaccacaccttcatcaggaatgaatatccgatgacctaatgattctgagcttggcaaaggtcttatctcccagctcgcccaggcccagtgttccaggaatgtgacctttgctgcagcagccgctggagggggcagaggggatgggctggaggttgagcaaacagagcagcagaaaaggcagttcctcttctccagtgccctccttccctgtctctgcctctccctcccttcctcaggcatcagagcggagacttcagggagaccagagcccagcttgccaggcactgagctagaagccctgcc

SEQ ID NO: 380 - DNA, 345 bp, synthetic construct
  source 1..345: mol_type = other DNA
caggctttcactttctcgccaacttacaaggcctttctgtgtaaacaatacctgaacctttaccccgttgcccggcaacggccaggtctgtgccaagtgtttgcctttgcaacagcttatcggaagcaaacaagctgaggggaattgagcaagaatttctgggataccaacagcataggaggaacaaaggacgtagagggagggttgactgtctacacaggacaaagccaatgattaaccaaacctcttgcagatttaaataggatgggaactaggagtggcagcaatcctttctttcagctggagtgctcctcaggagccagccccacccttagaaaagccacc

SEQ ID NO: 381 - DNA, 337 bp, synthetic construct
  source 1..337: mol_type = other DNA
aagcaaatatttgtggttatggattaactcgaactgtttgcccactctatttgccctgtaccggcccgggaggcgccctttggaccttttgcaatcctggcgcactgaacccttgacccctgccctgcagcccccgcagcttgctgtttgcccactctatttgcccagccccagccctggagagtcctttagcagggcaaagtgcaacataggcagaccttaagggatgactcagtaacagataagctttgtgtgcctgcagggcatataaaacaggggcaaggcacagactcatagcagagcaatcaccaccaagcctggaataactgcagccacc

SEQ ID NO: 382 - DNA, 326 bp, synthetic construct
  source 1..326: mol_type = other DNA
ggcgccctttggaccttttgcaatcctggagcaaacagcaaacactgtaccggcccgggaggcgccctttggaccttttgcaatcctggcgcactgaacccttgacccctgccctgcagcccccgcagcttgctgtttgcccactctatttgcccagccccagccctggagagtcctttagcagggcaaagtgcaacataggcagaccttaagggatgactcagtaacagataagctttgtgtgcctgcagggcatataaaacaggggcaaggcacagactcatagcagagcaatcaccaccaagcctggaataactgcagccacc

SEQ ID NO: 383 - DNA, 442 bp, synthetic construct
  source 1..442: mol_type = other DNA
aggttaatttttaaaaagcagtcaaaagtccaagtggcccttggcagcatttactctctctgtttgctctggttaataatctcaggagcacaaacattcctgtaccagaatgaacattgaactttggactatacctgaggggtgaggtaaacaacaggactataaatggcccgggaggcgccctttggaccttttgcaatcctggcgcactgaacccttgacccctgccctgcagcccccgcagcttgctgtttgcccactctatttgcccagccccagccctggagagtcctttagcagggcaaagtgcaacataggcagaccttaagggatgactcagtaacagataagctttgtgtgcctgcagggcatataaaacaggggcaaggcacagactcatagcagagcaatcaccaccaagcctggaataactgcagccacc

SEQ ID NO: 384 - DNA, 398 bp, synthetic construct
  source 1..398: mol_type = other DNA
aagcaaatatttgtggttatggattaactcgaactgtttgcccactctatttgccctgtaccagaatgaacattgaactttggactatacctgaggggtgaggtaaacaacaggactataaatggcccgggaggcgccctttggaccttttgcaatcctggcgcactgaacccttgacccctgccctgcagcccccgcagcttgctgtttgcccactctatttgcccagccccagccctggagagtcctttagcagggcaaagtgcaacataggcagaccttaagggatgactcagtaacagataagctttgtgtgcctgcagggcatataaaacaggggcaaggcacagactcatagcagagcaatcaccaccaagcctggaataactgcagccacc

SEQ ID NO: 385 - DNA, 387 bp, synthetic construct
  source 1..387: mol_type = other DNA
ggcgccctttggaccttttgcaatcctggagcaaacagcaaacactgtaccagaatgaacattgaactttggactatacctgaggggtgaggtaaacaacaggactataaatggcccgggaggcgccctttggaccttttgcaatcctggcgcactgaacccttgacccctgccctgcagcccccgcagcttgctgtttgcccactctatttgcccagccccagccctggagagtcctttagcagggcaaagtgcaacataggcagaccttaagggatgactcagtaacagataagctttgtgtgcctgcagggcatataaaacaggggcaaggcacagactcatagcagagcaatcaccaccaagcctggaataactgcagccacc

SEQ ID NO: 386 - DNA, 433 bp, synthetic construct
  source 1..433: mol_type = other DNA
aggttaatttttaaaaagcagtcaaaagtccaagtggcccttggcagcatttactctctctgtttgctctggttaataatctcaggagcacaaacattcctgtaccagaatgaacattgaactttggactatacctgaggggtgaggtaaacaacaggactataaatggcccgggaggcgccctttggaccttttgcaatcctggcgctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagatgcgccacc

SEQ ID NO: 387 - DNA, 389 bp, synthetic construct
  source 1..389: mol_type = other DNA
aagcaaatatttgtggttatggattaactcgaactgtttgcccactctatttgccctgtaccagaatgaacattgaactttggactatacctgaggggtgaggtaaacaacaggactataaatggcccgggaggcgccctttggaccttttgcaatcctggcgctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagatgcgccacc

SEQ ID NO: 388 - DNA, 378 bp, synthetic construct
  source 1..378: mol_type = other DNA
ggcgccctttggaccttttgcaatcctggagcaaacagcaaacactgtaccagaatgaacattgaactttggactatacctgaggggtgaggtaaacaacaggactataaatggcccgggaggcgccctttggaccttttgcaatcctggcgctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagatgcgccacc

SEQ ID NO: 389 - DNA, 611 bp, synthetic construct; identical to SEQ ID NO: 376
  source 1..611: mol_type = other DNA

SEQ ID NO: 390 - DNA, 580 bp, synthetic construct; identical to SEQ ID NO: 377
  source 1..580: mol_type = other DNA

SEQ ID NO: 391 - DNA, 410 bp, synthetic construct; identical to SEQ ID NO: 378
  source 1..410: mol_type = other DNA

SEQ ID NO: 392 - DNA, 512 bp, synthetic construct; identical to SEQ ID NO: 379
  source 1..512: mol_type = other DNA

SEQ ID NO: 393 - DNA, 345 bp, synthetic construct; identical to SEQ ID NO: 380
  source 1..345: mol_type = other DNA

SEQ ID NO: 394 - DNA, 381 bp, synthetic construct; identical to SEQ ID NO: 94
  source 1..381: mol_type = other DNA

SEQ ID NO: 395 - DNA, 337 bp, synthetic construct; identical to SEQ ID NO: 381
  source 1..337: mol_type = other DNA

SEQ ID NO: 396 - DNA, 326 bp, synthetic construct; identical to SEQ ID NO: 382
  source 1..326: mol_type = other DNA

SEQ ID NO: 397 - DNA, 442 bp, synthetic construct; identical to SEQ ID NO: 383
  source 1..442: mol_type = other DNA

SEQ ID NO: 398 - DNA, 398 bp, synthetic construct; identical to SEQ ID NO: 384
  source 1..398: mol_type = other DNA

SEQ ID NO: 399 - DNA, 387 bp, synthetic construct; identical to SEQ ID NO: 385
  source 1..387: mol_type = other DNA

SEQ ID NO: 400 - DNA, 433 bp, synthetic construct; identical to SEQ ID NO: 386
  source 1..433: mol_type = other DNA

SEQ ID NO: 401 - DNA, 389 bp, synthetic construct; identical to SEQ ID NO: 387
  source 1..389: mol_type = other DNA

SEQ ID NO: 402 - DNA, 378 bp, synthetic construct; identical to SEQ ID NO: 388
  source 1..378: mol_type = other DNA

SEQ ID NO: 403 - DNA, 203 bp, synthetic construct
  source 1..203: mol_type = other DNA
aggttaatttttaaaaagcagtcaaaagtccaagtggcccttggcagcatttactctctctgtttgctctggttaataatctcaggagcacaaacattccagtcatatgtttgctcactgaaggttactagttaacaggcatcccttaaacaggatataaaaggacttcagcaggactgctcgaaacatcccactcagccacc

SEQ ID NO: 404 - DNA, 222 bp, synthetic construct
  source 1..222: mol_type = other DNA
aggttaatttttaaaaagcagtcaaaagtccaagtggcccttggcagcatttactctctctgtttgctctggttaataatctcaggagcacaaacattcctgtaccggcccgggaggcgccctttggaccttttgcaatcctggcggggcatataaaacaggggcaaggcacagactcatagcagagcaatcaccaccaagcctggaataactgcagccacc

SEQ ID NO: 405 - DNA, 275 bp, synthetic construct
  source 1..275: mol_type = other DNA
tgctctctgacaaagatacggtgggtcccactgatgaactgtgctgccacagtaaatgtagccactatgcctatctccattctgaagatgtgccctgttcaaacatgtcctaatactctgtctctgcaagggtcatcagtagttttccatcttactcaacatcctcccagtgagtcatatgtttgctcactgaaggttactagttaacaggcatcccttaaacaggatataaaaggacttcagcaggactgctcgaaacatcccactcagccacc

SEQ ID NO: 406 - DNA, 283 bp, synthetic construct
  source 1..283: mol_type = other DNA
cctccccgtgttcctgctctttgtccctctgtcctacttagactaatatttgccttgggtactgcaaacaggaaatgggggagggacaggagtagggcggccctgttcaaacatgtcctaatactctgtctctgcaagggtcatcagtagttttccatcttactcaacatcctcccagtgagtcatatgtttgctcactgaaggttactagttaacaggcatcccttaaacaggatataaaaggacttcagcaggactgctcgaaacatcccactcagccacc

SEQ ID NO: 407 - DNA, 262 bp, synthetic construct
  source 1..262: mol_type = other DNA
ccgcccccactgaacccttgacccctgccctgcagcccccgcagcttgctgtttgcccactctatttgcccagccccagccctgttcaaacatgtcctaatactctgtctctgcaagggtcatcagtagttttccatcttactcaacatcctcccagtgagtcatatgtttgctcactgaaggttactagttaacaggcatcccttaaacaggatataaaaggacttcagcaggactgctcgaaacatcccactcagccacc

SEQ ID NO: 408 - DNA, 268 bp, synthetic construct
  source 1..268: mol_type = other DNA
aagctttctgaacagccaaacagagattccaaagttcaggcaccaaagttcagaccctaacagttatttacaagggtcagttaacccctgttcaaacatgtcctaatactctgtctctgcaagggtcatcagtagttttccatcttactcaacatcctcccagtgagtcatatgtttgctcactgaaggttactagttaacaggcatcccttaaacaggatataaaaggacttcagcaggactgctcgaaacatcccactcagccacc

SEQ ID NO: 409 - DNA, 389 bp, synthetic construct; identical to SEQ ID NO: 387
  source 1..389: mol_type = other DNA

SEQ ID NO: 410 - DNA, 378 bp, synthetic construct; identical to SEQ ID NO: 388
  source 1..378: mol_type = other DNA

SEQ ID NO: 411 - DNA, 203 bp, synthetic construct; identical to SEQ ID NO: 403
  source 1..203: mol_type = other DNA

SEQ ID NO: 412 - DNA, 222 bp, synthetic construct; identical to SEQ ID NO: 404
  source 1..222: mol_type = other DNA

SEQ ID NO: 413 - DNA, 275 bp, synthetic construct; identical to SEQ ID NO: 405
  source 1..275: mol_type = other DNA

SEQ ID NO: 414 - DNA, 283 bp, synthetic construct; identical to SEQ ID NO: 406
  source 1..283: mol_type = other DNA

SEQ ID NO: 415 - DNA, 262 bp, synthetic construct; identical to SEQ ID NO: 407
  source 1..262: mol_type = other DNA

SEQ ID NO: 416 - DNA, 268 bp, synthetic construct; identical to SEQ ID NO: 408
  source 1..268: mol_type = other DNA

SEQ ID NO: 417 - DNA, 422 bp, synthetic construct
  source 1..422: mol_type = other DNA
aggttaatttttaaaaagcagtcaaaagtccaagtggcccttggcagcatttactctctctgtttgctctggttaataatctcaggagcacaaacattccggcccgggaggcgccctttggaccttttgcaatcctggcgccgatgacctaatgattctgagcttggcaaaggtcttatctcccagctcgcccaggcccagtgttccaggaatgtgacctttgctgcagcagccgctggagggggcagaggggatgggctggaggttgagcaaacagagcagcagaaaaggcagttcctcttctccagtgccctccttccctgtctctgcctctccctcccttcctcaggcatcagagcggagacttcagggagaccagagcccagcttgccaggcactgagctagaagccctgccgccacc

SEQ ID NO: 418 - DNA, 176 bp, synthetic construct
  source 1..176: mol_type = other DNA
aggttaatttttaaaaagcagtcaaaagtccaagtggcccttggcagcatttactctctctgtttgctctggttaataatctcaggagcacaaacattccgggcatataaaacaggggcaaggcacagactcatagcagagcaatcaccaccaagcctggaataactgcagccacc

SEQ ID NO: 419 - DNA, 309 bp, synthetic construct
  source 1..309: mol_type = other DNA
aggttaatttttaaaaagcagtcaaaagtccaagtggcccttggcagcatttactctctctgtttgctctggttaataatctcaggagcacaaacattcctgtaccggcccgggaggcgccctttggaccttttgcaatcctggcgccctggagagtcctttagcagggcaaagtgcaacataggcagaccttaagggatgactcagtaacagataagctttgtgtgcctgcagggcatataaaacaggggcaaggcacagactcatagcagagcaatcaccaccaagcctggaataactgcagccacc

SEQ ID NO: 420 - DNA, 341 bp, synthetic construct
  source 1..341: mol_type = other DNA
aggttaatttttaaaaagcagtcaaaagtccaagtggcccttggcagcatttactctctctgtttgctctggttaataatctcaggagcacaaacattcctgtacccactgaacccttgacccctgccctgcagcccccgcagcttgctgtttgcccactctatttgcccagccccagccctggagagtcctttagcagggcaaagtgcaacataggcagaccttaagggatgactcagtaacagataagctttgtgtgcctgcagggcatataaaacaggggcaaggcacagactcatagcagagcaatcaccaccaagcctggaataactgcagccacc

SEQ ID NO: 421 - DNA, 545 bp, synthetic construct
  source 1..545: mol_type = other DNA
caggctttcactttctcgccaacttacaaggcctttctgtgtaaacaatacctgaacctttaccccgttgcccggcaacggccaggtctgtgccaagtgtttgaggttaatttttaaaaagcagtcaaaagtccaagtggcccttggcagcatttactctctctgtttgctctggttaataatctcaggagcacaaacattcctgtaccagaatgaacattgaactttggactatacctgaggggtgaggtaaacaacaggactataaatggcccgggaggcgccctttggaccttttgcaatcctggcgcactgaacccttgacccctgccctgcagcccccgcagcttgctgtttgcccactctatttgcccagccccagccctggagagtcctttagcagggcaaagtgcaacataggcagaccttaagggatgactcagtaacagataagctttgtgtgcctgcagggcatataaaacaggggcaaggcacagactcatagcagagcaatcaccaccaagcctggaataactgcagccacc

SEQ ID NO: 422 - DNA, 137 bp, synthetic construct
  source 1..137: mol_type = other DNA
agaatgaacattgaactttggactatacctgaggggtgaggtaaacaacaggactataaatgggcatataaaacaggggcaaggcacagactcatagcagagcaatcaccaccaagcctggaataactgcagccacc

SEQ ID NO: 423 - DNA, 440 bp, synthetic construct
  source 1..440: mol_type = other DNA
caggctttcactttctcgccaacttacaaggcctttctgtgtaaacaatacctgaacctttaccccgttgcccggcaacggccaggtctgtgccaagtgtttgaagcaaatatttgtggttatggattaactcgaactgtttgcccactctatttgccctgtaccggcccgggaggcgccctttggaccttttgcaatcctggcgcactgaacccttgacccctgccctgcagcccccgcagcttgctgtttgcccactctatttgcccagccccagccctggagagtcctttagcagggcaaagtgcaacataggcagaccttaagggatgactcagtaacagataagctttgtgtgcctgcagggcatataaaacaggggcaaggcacagactcatagcagagcaatcaccaccaagcctggaataactgcagccacc

SEQ ID NO: 424 - DNA, 501 bp, synthetic construct
  source 1..501: mol_type = other DNA
caggctttcactttctcgccaacttacaaggcctttctgtgtaaacaatacctgaacctttaccccgttgcccggcaacggccaggtctgtgccaagtgtttgaagcaaatatttgtggttatggattaactcgaactgtttgcccactctatttgccctgtaccagaatgaacattgaactttggactatacctgaggggtgaggtaaacaacaggactataaatggcccgggaggcgccctttggaccttttgcaatcctggcgcactgaacccttgacccctgccctgcagcccccgcagcttgctgtttgcccactctatttgcccagccccagccctggagagtcctttagcagggcaaagtgcaacataggcagaccttaagggatgactcagtaacagataagctttgtgtgcctgcagggcatataaaacaggggcaaggcacagactcatagcagagcaatcaccaccaagcctggaataactgcagccacc

SEQ ID NO: 425 - DNA, 414 bp, synthetic construct
  source 1..414: mol_type = other DNA
caggctttcactttctcgccaacttacaaggcctttctgtgtaaacaatacctgaacctttaccccgttgcccggcaacggccaggtctgtgccaagtgtttgaagcaaatatttgtggttatggattaactcgaactgtttgcccactctatttgccctgtaccagaatgaacattgaactttggactatacctgaggggtgaggtaaacaacaggactataaatggcccgggaggcgccctttggaccttttgcaatcctggcgcactgaacccttgacccctgccctgcagcccccgcagcttgctgtttgcccactctatttgcccagccccaggggcatataaaacaggggcaaggcacagactcatagcagagcaatcaccaccaagcctggaataactgcagccacc

SEQ ID NO: 426 - DNA, 243 bp, synthetic construct
  source 1..243: mol_type = other DNA
aggttaatttttaaaaagcagtcaaaagtccaagtggcccttggcagcatttactctctctgtttgctctggttaataatctcaggagcacaaacattccggcccgggaggcgccctttggaccttttgcaatcctggcgagtcatatgtttgctcactgaaggttactagttaacaggcatcccttaaacaggatataaaaggacttcagcaggactgctcgaaacatcccactcagccacc

SEQ ID NO: 427 - DNA, 279 bp, synthetic construct
  source 1..279: mol_type = other DNA
aggttaatttttaaaaagcagtcaaaagtccaagtggcccttggcagcatttactctctctgtttgctctggttaataatctcaggagcacaaacattcccaggctttcactttctcgccaacttacaaggcctttctgtgtaaacaatacctgaacctttaccccgttgcccggcaacggccaggtctgtgccaagtgtttggggcatataaaacaggggcaaggcacagactcatagcagagcaatcaccaccaagcctggaataactgcagccacc

SEQ ID NO: 428 - DNA, 256 bp, synthetic construct
  source 1..256: mol_type = other DNA
aggttaatttttaaaaagcagtcaaaagtccaagtggcccttggcagcatttactctctctgtttgctctggttaataatctcaggagcacaaacattccccctgttcaaacatgtcctaatactctgtctctgcaagggtcatcagtagttttccatcttactcaacatcctcccagtggggcatataaaacaggggcaaggcacagactcatagcagagcaatcaccaccaagcctggaataactgcagccacc

SEQ ID NO: 429 - DNA, 183 bp, synthetic construct
  source 1..183: mol_type = other DNA
ccctgttcaaacatgtcctaatactctgtctctgcaagggtcatcagtagttttccatcttactcaacatcctcccagtgagtcatatgtttgctcactgaaggttactagttaacaggcatcccttaaacaggatataaaaggacttcagcaggactgctcgaaacatcccactcagccacc

SEQ ID NO: 430 - DNA, 249 bp, synthetic construct
  source 1..249: mol_type = other DNA
aggttaatttttaaaaagcagtcaaaagtccaagtggcccttggcagcatttactctctctgtttgctctggttaataatctcaggagcacaaacattcctgtaccggcccgggaggcgccctttggaccttttgcaatcctggcgagtcatatgtttgctcactgaaggttactagttaacaggcatcccttaaacaggatataaaaggacttcagcaggactgctcgaaacatcccactcagccacc

SEQ ID NO: 431 - DNA, 349 bp, synthetic construct
  source 1..349: mol_type = other DNA
tcgagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacaga

SEQ ID NO: 432 - DNA, 558 bp, synthetic construct
  source 1..558: mol_type = other DNA
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttctctcttttagattccaacctttggaactgaattctagaccacc

SEQ ID NO: 433 - DNA, 580 bp, Cytomegalovirus sp.
  source 1..580: mol_type = genomic DNA
atagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatc

SEQ ID NO: 434 - DNA, 1775 bp, synthetic construct
  source 1..1775: mol_type = other DNA
agatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcctcgaagatctaggcaacgcgtctcgaggcggccgccgccacc

SEQ ID NO: 435 - DNA, 890 bp, synthetic construct
  source 1..890: mol_type = other DNA
aggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagactgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacagggcaatccggtactgttggtaaagccacc

SEQ ID NO: 436-437 - skipped (2 entries)

SEQ ID NO: 438 - DNA, 290 bp, synthetic construct
  source 1..290: mol_type = other DNA
tcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgaacggtggagggcagtgtagtctgagcagtactcgttgctgccgcgcgcgccaccagacataatagctgacagactaacagactgttcctttccatgggtcttttctgcaggccacc

SEQ ID NO: 439 - DNA, 544 bp, synthetic construct
  source 1..544: mol_type = other DNA
tgaaatgcctgccatatattagtgccctgaagtccaaaggtagaggaaccgagtgtttaaaaattactgtggctgtggagtcaacatgatgtaaaaaaacaaacatttggataacaccaagaagccagatatggttgaaatgttgactggttgacaaaaataatttgggttgcttaatggtgcacaaaggtaatgcaaaaaggttaatttttaaaaagcagtcaaaagtccaagtggcccttggcagcatttactctctctgtttgctctggttaataatctcaggagcacaaacattccggcccgggaggcgccctttggaccttttgcaatcctggcgcactgaacccttgacccctgccctgcagcccccgcagcttgctgtttgcccactctatttgcccagccccagccctggagagtcctttagcagggcaaagtgcaacataggcagaccttaagggatgactcagtaacagataagctttgtgtgcctgcagggcatataaaacaggggcaaggcacagactcatagcagagcaat

SEQ ID NO: 440 - DNA, 275 bp, synthetic construct
  source 1..275: mol_type = other DNA
aggttaatttttaaaaagcagtcaaaagtccaagtggcccttggcagcatttactctctctgtttgctctggttaataatctcaggagcacaaacattccccctgttcaaacatgtcctaatactctgtctctgcaagggtcatcagtagttttccatcttactcaacatcctcccagtgagtcatatgtttgctcactgaaggttactagttaacaggcatcccttaaacaggatataaaaggacttcagcaggactgctcgaaacatcccact

SEQ ID NO: 441 - DNA, 350 bp, synthetic construct
  source 1..350: mol_type = other DNA
aggttaatttttaaaaagcagtcaaaagtccaagtggcccttggcagcatttactctctctgtttgctctggttaataatctcaggagcacaaacattcctgtaccggcccgggaggcgccctttggaccttttgcaatcctggcgcactgaacccttgacccctgccctgcagcccccgcagcttgctgtttgcccactctatttgcccagccccagccctggagagtcctttagcagggcaaagtgcaacataggcagaccttaagggatgactcagtaacagataagctttgtgtgcctgcagggcatataaaacaggggcaaggcacagactcatagcagagcaat

SEQ ID NO: 442 - DNA, 85 bp, unidentified
  source 1..85: mol_type = unassigned DNA
ctagccctaaggtaagttggcgccgtttaagggatggttggttggtggggtattaatgtttaattaccttttttacaggcctgaa

SEQ ID NO: 443 - DNA, 441 bp, Homo sapiens
  source 1..441: mol_type = genomic DNA
gtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaag

SEQ ID NO: 444 - DNA, 179 bp, Homo sapiens
  source 1..179: mol_type = genomic DNA
tactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaatt

SEQ ID NO: 445 - DNA, 818 bp, unidentified
  source 1..818: mol_type = unassigned DNA
acttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatgcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagac

SEQ ID NO: 446 - DNA, 173 bp, synthetic construct
  source 1..173: mol_type = other DNA
caagagtgacgtaagtaccgcctatagactctataggcacacccctttggctcttatgcatgaacggtggagggcagtgtagtctgagcagtactcgttgctgccgcgcgcgccaccagacataatagctgacagactaacagactgttcctttccatgggtcttttctgcag

SEQ ID NO: 447 - skipped

SEQ ID NO: 448 - DNA, 145 bp, synthetic construct; identical to SEQ ID NO: 105
  source 1..145: mol_type = other DNA

SEQ ID NO: 449 - DNA, 136 bp, synthetic construct
  source 1..136: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagaggg

SEQ ID NO: 450 - DNA, 76 bp, synthetic construct
  source 1..76: mol_type = other DNA
gggcatataaaacaggggcaaggcacagactcatagcagagcaatcaccaccaagcctggaataactgcagccacc

SEQ ID NO: 451 - skipped

SEQ ID NO: 452 - protein, 736 aa, synthetic construct
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 453 - DNA, 2208 bp, synthetic construct
  source 1..2208: mol_type = other DNA
atggctgctgacggttatcttccagattggctcgaggacaacctttctgaaggcattcgtgagtggtgggctctgaaacctggagtccctcaacccaaagcgaaccaacaacaccaggacaaccgtcggggtcttgtgcttccgggttacaaatacctcggacccggtaacggactcgacaaaggagagccggtcaacgaggcggacgcggcagccctcgaacacgacaaagcttacgaccagcagctcaaggccggtgacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagcagctaaaacggctcctggaaagaagaggcctgtagatcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcagatgcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttg

SEQ ID NO: 454 - protein, 736 aa, synthetic construct
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSDASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 455 - DNA, 2208 bp, synthetic construct
  source 1..2208: mol_type = other DNA
atggctgctgacggttatcttccagattggctcgaggacaacctttctgaaggcattcgtgagtggtgggctctgaaacctggagtccctcaacccaaagcgaaccaacaacaccaggacaaccgtcggggtcttgtgcttccgggttacaaatacctcggacccggtaacggactcgacaaaggagagccggtcaacgaggcggacgcggcagccctcgaacacgacaaagcttacgaccagcagctcaaggccggtgacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagcagctaaaacggctcctggaaagaagaggcctgtagatcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcagatgcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaaggtagctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcgtaccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttg

SEQ ID NO: 456 - protein, 736 aa, synthetic construct
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKVANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFVPAKFASFITQYSTGQVSVEIEWELQKENSKRWNPEIQYTSNYNKSVNVDFTVDTNGVYSEPRPIGTRYLTRNL

SEQ ID NO: 457 - DNA, 2134 bp, synthetic construct
  source 1..2134: mol_type = other DNA
gaaacctggagtccctcaacccaaagcgaaccaacaacaccaggacaaccgtcggggtcttgtgcttccgggttacaaatacctcggacccggtaacggactcgacaaaggagagccggtcaacgaggcggacgcggcagccctcgaacacgacaaagcttacgaccagcagctcaaggccggtgacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagcagctaaaacggctcctggaaagaagaggcctgtagatcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttg

SEQ ID NO: 458 - protein, 736 aa, synthetic construct
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSDASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTAASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 459 - DNA, 2208 bp, synthetic construct
  source 1..2208: mol_type = other DNA
atggctgctgacggttatcttccagattggctcgaggacaacctttctgaaggcattcgtgagtggtgggctctgaaacctggagtccctcaacccaaagcgaaccaacaacaccaggacaaccgtcggggtcttgtgcttccgggttacaaatacctcggacccggtaacggactcgacaaaggagagccggtcaacgaggcggacgcggcagccctcgaacacgacaaagcttacgaccagcagctcaaggccggtgacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagcagctaaaacggctcctggaaagaagaggcctgtagatcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttg

SEQ ID NO: 460 - protein, 736 aa, synthetic construct
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTAASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 461 - DNA, 2208 bp, synthetic construct; identical to SEQ ID NO: 459
  source 1..2208: mol_type = other DNA

SEQ ID NO: 462 - protein, 736 aa, synthetic construct
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSYSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 463 - DNA, 2208 bp, synthetic construct; identical to SEQ ID NO: 459
  source 1..2208: mol_type = other DNA

SEQ ID NO: 464 - skipped

SEQ ID NO: 465 - DNA, 12 bp, synthetic construct
  source 1..12: mol_type = other DNA
tgataactcgag

SEQ ID NO: 466 - DNA, 21 bp, synthetic construct
  source 1..21: mol_type = other DNA
ggaacccctagtgatggagtt

SEQ ID NO: 467 - DNA, 16 bp, synthetic construct
  source 1..16: mol_type = other DNA
cggcctcagtgagcga

SEQ ID NO: 468 - DNA, 21 bp, synthetic construct
  source 1..21: mol_type = other DNA
  modified_base 1: mod_base = OTHER; note = Fluorescein modified nucleotide
  modified_base 21: mod_base = OTHER; note = 3'black hole quencher-1 modified nucleotide
cactccctctctgcgcgctcg

SEQ ID NO: 469 - DNA, 24 bp, synthetic construct
  source 1..24: mol_type = other DNA
  modified_base 24: mod_base = OTHER; note = Fluorescein modified nucleotide
cccctgggcatggagagcaaggcc

SEQ ID NO: 470 - DNA, 20 bp, synthetic construct
  source 1..20: mol_type = other DNA
gatgggctgtgacctgaaca

SEQ ID NO: 471 - DNA, 20 bp, synthetic construct
  source 1..20: mol_type = other DNA
ccaggtggcaaacatgttgg

SEQ ID NO: 472 - DNA, 24 bp, synthetic construct
  source 1..24: mol_type = other DNA
  modified_base 1: mod_base = OTHER; note = 5'Hexachlorofluorescein modified nucleotide
ttgtcatcaacgggaagcccatca

SEQ ID NO: 473 - DNA, 19 bp, synthetic construct
  source 1..19: mol_type = other DNA
acggcaaattcaacggcac

SEQ ID NO: 474 - DNA, 20 bp, synthetic construct
  source 1..20: mol_type = other DNA
tagtggggtctcgctcctgg

SEQ ID NO: 475 - DNA, 24 bp, synthetic construct
  source 1..24: mol_type = other DNA
  modified_base 24: mod_base = OTHER; note = Fluorescein modified nucleotide
cccctgggcatggagagcaaggcc

SEQ ID NO: 476 - DNA, 20 bp, synthetic construct
  source 1..20: mol_type = other DNA
gatgggctgtgacctgaaca

SEQ ID NO: 477 - DNA, 20 bp, synthetic construct
  source 1..20: mol_type = other DNA
ccaggtggcaaacatgttgg

SEQ ID NO: 478 - DNA, 14 bp, synthetic construct
  source 1..14: mol_type = other DNA
  modified_base 1: mod_base = OTHER; note = thymine attached to 5'Hexachlorofluorescein-tggagggtg-ZEN quencher
tccgcaacgtggcc

SEQ ID NO: 479 - DNA, 20 bp, synthetic construct
  source 1..20: mol_type = other DNA
caggtgtttgacaacggcag

SEQ ID NO: 480 - DNA, 20 bp, synthetic construct
  source 1..20: mol_type = other DNA
ccgatctgcagacacacact

SEQ ID NO: 481 - DNA, 282 bp, synthetic construct
  source 1..282: mol_type = other DNA
aggttaatttttaaaaagcagtcaaaagtccaagtggcccttggcagcatttactctctctgtttgctctggttaataatctcaggagcacaaacattccggcccgggaggcgccctttggaccttttgcaatcctggcgcactgaacccttgacccctgccctgcagcccccgcagcttgctgtttgcccactctatttgcccagccccaggggcatataaaacaggggcaaggcacagactcatagcagagcaatcaccaccaagcctggaataactgca

SEQ ID NO: 482 - DNA, 277 bp, synthetic construct
  source 1..277: mol_type = other DNA
aggttaatttttaaaaagcagtcaaaagtccaagtggcccttggcagcatttactctctctgtttgctctggttaataatctcaggagcacaaacattccccctgttcaaacatgtcctaatactctgtctctgcaagggtcatcagtagttttccatcttactcaacatcctcccagtgagtcatatgtttgctcactgaaggttactagttaacaggcatcccttaaacaggatataaaaggacttcagcaggactgctcgaaacatcccactca

SEQ ID NO: 483 - DNA, 268 bp, synthetic construct
  source 1..268: mol_type = other DNA
ccctgttcaaacatgtcctaatactctgtctctgcaagggtcatcagtagttttccatcttactcaacatcctcccagtgcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcg

SEQ ID NO: 484 - DNA, 254 bp, synthetic construct
  source 1..254: mol_type = other DNA
ccgcccccactgaacccttgacccctgccctgcagcccccgcagcttgctgtttgcccactctatttgcccagccccagccctgttcaaacatgtcctaatactctgtctctgcaagggtcatcagtagttttccatcttactcaacatcctcccagtgagtcatatgtttgctcactgaaggttactagttaacaggcatcccttaaacaggatataaaaggacttcagcaggactgctcgaaacatcccact

SEQ ID NO: 485 - DNA, 260 bp, synthetic construct
  source 1..260: mol_type = other DNA
aagctttctgaacagccaaacagagattccaaagttcaggcaccaaagttcagaccctaacagttatttacaagggtcagttaacccctgttcaaacatgtcctaatactctgtctctgcaagggtcatcagtagttttccatcttactcaacatcctcccagtgagtcatatgtttgctcactgaaggttactagttaacaggcatcccttaaacaggatataaaaggacttcagcaggactgctcgaaacatcccact

SEQ ID NO: 486 - DNA, 250 bp, synthetic construct
  source 1..250: mol_type = other DNA
aggttaatttttaaaaagcagtcaaaagtccaagtggcccttggcagcatttactctctctgtttgctctggttaataatctcaggagcacaaacattccccctgttcaaacatgtcctaatactctgtctctgcaagggtcatcagtagttttccatcttactcaacatcctcccagtggggcatataaaacaggggcaaggcacagactcatagcagagcaatcaccaccaagcctggaataactgca

SEQ ID NO: 487 - DNA, 175 bp, synthetic construct
  source 1..175: mol_type = other DNA
ccctgttcaaacatgtcctaatactctgtctctgcaagggtcatcagtagttttccatcttactcaacatcctcccagtgagtcatatgtttgctcactgaaggttactagttaacaggcatcccttaaacaggatataaaaggacttcagcaggactgctcgaaacatcccact

SEQ ID NO: 488 - DNA, 318 bp, synthetic construct
  source 1..318: mol_type = other DNA
aggttaatttttaaaaagcagtcaaaagtccaagtggcccttggcagcatttactctctctgtttgctctggttaataatctcaggagcacaaacattccccctgttcaaacatgtcctaatactctgtctctgcaagggtcatcagtagttttccatcttactcaacatcctcccagtgagtcatatgtttgctcactgaaggttactagttaacaggcatcccttaaacaggatataaaaggacttcagcaggactgctcgaaacatcccactcaggtggattcttgggcatttgcttaagcttggtaccgccacc

SEQ ID NO: 489 - DNA, 229 bp, synthetic construct
  source 1..229: mol_type = other DNA
aggttaatttttaaaaagcagtcaaaagtccaagtggcccttggcagcatttactctctctgtttgctctggttaataatctcaggagcacaaacattccggcccgggaggcgccctttggaccttttgcaatcctggcgtctagagggtatataatgggggccactagtgccagcagcagcctgaccacatctcatcctcaatcaccaccaagcctggaataactgca

SEQ ID NO: 490 - DNA, 214 bp, synthetic construct
  source 1..214: mol_type = other DNA
aggttaatttttaaaaagcagtcaaaagtccaagtggcccttggcagcatttactctctctgtttgctctggttaataatctcaggagcacaaacattccggcccgggaggcgccctttggaccttttgcaatcctggcgtctagagggtatataatgggggccagccgctggcgtttccgttgcttggatcagtctaggtgcagctgccggat

SEQ ID NO: 491 - DNA, 226 bp, synthetic construct
  source 1..226: mol_type = other DNA
ccctgttcaaacatgtcctaatactctgtctctgcaagggtcatcagtagttttccatcttactcaacatcctcccagtgcgggcgactcagatcccagccagtggacttagcccctgtttgctcctccgataactggggtgaccttggttaatattcaccagcagcctcccccgttgcccctctggatccactgcttaaatacggacgaggacagggccctgtct

SEQ ID NO: 492 - DNA, 246 bp, synthetic construct
  source 1..246: mol_type = other DNA
aggttaatttttaaaaagcagtcaaaagtccaagtggcccttggcagcatttactctctctgtttgctctggttaataatctcaggagcacaaacattcccgggcgactcagatcccagccagtggacttagcccctgtttgctcctccgataactggggtgaccttggttaatattcaccagcagcctcccccgttgcccctctggatccactgcttaaatacggacgaggacagggccctgtct

SEQ ID NO: 493 - DNA, 288 bp, synthetic construct
  source 1..288: mol_type = other DNA
aggttaatttttaaaaagcagtcaaaagtccaagtggcccttggcagcatttactctctctgtttgctctggttaataatctcaggagcacaaacattcccgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcg

SEQ ID NO: 494 - DNA, 290 bp, synthetic construct
  source 1..290: mol_type = other DNA
aggttaatttttaaaaagcagtcaaaagtccaagtggcccttggcagcatttactctctctgtttgctctggttaataatctcaggagcacaaacattcctgtaccggcccgggaggcgccctttggaccttttgcaatcctggcgggcgactcagatcccagccagtggacttagcccctgtttgctcctccgataactggggtgaccttggttaatattcaccagcagcctcccccgttgcccctctggatccactgcttaaatacggacgaggacagggccctgtct

SEQ ID NO: 495 - DNA, 247 bp, synthetic construct
  source 1..247: mol_type = other DNA
cctgaagtccaaaggtagaggaaccgagtgtttaaaaattactgtggctgtggagtcaacatgatgtaaaaaaacaaacatttggataacaccaagaagccagatatggttgaaatgttgactggttgacaaaaataatttgggttgcttaatggtgcacaaaggtaatgcgggcatataaaacaggggcaaggcacagactcatagcagagcaatcaccaccaagcctggaataactgcagccacc

SEQ ID NO: 496 - DNA, 326 bp, synthetic construct
  source 1..326: mol_type = other DNA
ccgcccccactgaacccttgacccctgccctgcagcccccgcagcttgctgtttgcccactctatttgcccagccccagcctgaagtccaaaggtagaggaaccgagtgtttaaaaattactgtggctgtggagtcaacatgatgtaaaaaaacaaacatttggataacaccaagaagccagatatggttgaaatgttgactggttgacaaaaataatttgggttgcttaatggtgcacaaaggtaatgcgggcatataaaacaggggcaaggcacagactcatagcagagcaatcaccaccaagcctggaataactgcagccacc

SEQ ID NO: 497 - DNA, 345 bp, synthetic construct
  source 1..345: mol_type = other DNA
ccgcccccactgaacccttgacccctgccctgcagcccccgcagcttgctgtttgcccactctatttgcccagccccagcctgaagtccaaaggtagaggaaccgagtgtttaaaaattactgtggctgtggagtcaacatgatgtaaaaaaacaaacatttggataacaccaagaagccagatatggttgaaatgttgactggttgacaaaaataatttgggttgcttaatggtgcacaaaggtaatgcagtcatatgtttgctcactgaaggttactagttaacaggcatcccttaaacaggatataaaaggacttcagcaggactgctcgaaacatcccact

SEQ ID NO: 498 - DNA, 316 bp, synthetic construct
  source 1..316: mol_type = other DNA
tgctctctgacaaagatacggtgggtcccactgatgaactgtgctgccacagtaaatgtagccactatgcctatctccattctgaagatgtgccgcccccactgaacccttgacccctgccctgcagcccccgcagcttgctgtttgcccactctatttgcccagccccaggggcgactcagatcccagccagtggacttagcccctgtttgctcctccgataactggggtgaccttggttaatattcaccagcagcctcccccgttgcccctctggatccactgcttaaatacggacgaggacagggccctgtct

SEQ ID NO: 499 - DNA, 247 bp, synthetic construct
  source 1..247: mol_type = other DNA
tgctctctgacaaagatacggtgggtcccactgatgaactgtgctgccacagtaaatgtagccactatgcctatctccattctgaagatgtgccgcccccactgaacccttgacccctgccctgcagcccccgcagcttgctgtttgcccactctatttgcccagccccaggggcatataaaacaggggcaaggcacagactcatagcagagcaatcaccaccaagcctggaataactgcagccacc

SEQ ID NO: 500 - DNA, 266 bp, synthetic construct
  source 1..266: mol_type = other DNA
tgctctctgacaaagatacggtgggtcccactgatgaactgtgctgccacagtaaatgtagccactatgcctatctccattctgaagatgtgccgcccccactgaacccttgacccctgccctgcagcccccgcagcttgctgtttgcccactctatttgcccagccccagagtcatatgtttgctcactgaaggttactagttaacaggcatcccttaaacaggatataaaaggacttcagcaggactgctcgaaacatcccact

SEQ ID NO: 501 - DNA, 26 bp, synthetic construct
  source 1..26: mol_type = other DNA
  modified_base 26: mod_base = OTHER; note = Fluorescein modified nucleotide
gtttgaaactgtggagatgctgccct

SEQ ID NO: 502 - DNA, 20 bp, synthetic construct
  source 1..20: mol_type = other DNA
ccacttctctgggcatgtgt

SEQ ID NO: 503 - DNA, 20 bp, synthetic construct
  source 1..20: mol_type = other DNA
cctccagatgccagccttag

SEQ ID NO: 504 - DNA, 87 bp, synthetic construct
  source 1..87: mol_type = other DNA
  misc_difference 12..21: note = a, c, t, g, unknown or other
  misc_difference 12..21: note = This region may encompass 1-10 nucleotides
  misc_difference 33..42: note = a, c, t, g, unknown or other
  misc_difference 33..42: note = This region may encompass 1-10 nucleotides
  misc_difference 55..77: note = a, c, t, g, unknown or other
  misc_difference 55..77: note = This region may encompass 8-23 nucleotides
gttcaaacatgnnnnnnnnnnctaatactctgnnnnnnnnnntgcaagggtcatnnnnnnnnnnnnnnnnnnnnnnnttactcaaca

SEQ ID NO: 505 - DNA, 8218 bp, synthetic construct
  source 1..8218: mol_type = other DNA
tctagagctagcatatggatccatcgatttagggataacagggtaattatcagcacacaattgcccattatacgcgcgtataatggactattgtgtgctgatatctgtacacttaagggctagatcttagcttacgtcactagagggtccacgtttagtttttaagatccattgatctcctaaacgctgcaagattcgcaacctggtatacttagcctaggcgctaggtcctagtgcagcgggactttttttctaaagtcgttgagaggaggagtcgtcagaccagatagctttgatgtcctgatcggaaggatcgttggcccccctgcaggcagctgttaattaactgcgcgctcgctcgctcactgaggccgcccgggcaaagcccgggcgtcgggcgacctttggtcgcccggcctcagtgagcgagcgagcgcgcagagagggagtggccaactccatcactaggggttcctgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgccaccatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaaccccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactgaaataaaagatctttattttcattagatctgtgtgttggttttttgtgtgaggaacccctagtgatggagttggccactccctctctgcgcgctcgctcgctcactgaggccgggcgaccaaaggtcgcccgacgcccgggctttgcccgggcggcctcagtgagcgagcgagcgcgcagttaattaaggcgccctaggccgacccttagactctgtactcagttctataaacgagccattggatacgagatccgtagattgataagggacacggaatatccccggacgcaatagacaccggtggacagcttggtatcctgagcacagtcgcgcgtccgaatctagctctactttagaggccccggattctgatggtcgtagaccgcagaaccgattggggggatgagatctactagttatcagcacacaattgcccattatacgcgcgtataatggactattgtgtgctgatatagggataacagggtaattctagagctagcatatggatccatcgatttgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagcgtataatggtctagagctagcatatggatccatcgattccattatacgcctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 506-513 - skipped (8 entries)

SEQ ID NO: 514 - DNA, 49 bp, synthetic construct
  source 1..49: mol_type = other DNA
aataaaagatctttattttcattagatctgtgtgttggttttttgtgtg

SEQ ID NO: 515 - DNA, 4374 bp, synthetic construct
  source 1..4374: mol_type = other DNA
atgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgcagatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagcagggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaaccccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactga

SEQ ID NO: 516 - DNA, 208 bp, synthetic construct; identical to SEQ ID NO: 100
  source 1..208: mol_type = other DNA

SEQ ID NO: 517 - DNA, 199 bp, synthetic construct; identical to SEQ ID NO: 101
  source 1..199: mol_type = other DNA

SEQ ID NO: 518 - DNA, 154 bp, synthetic construct; identical to SEQ ID NO: 102
  source 1..154: mol_type = other DNA

SEQ ID NO: 519 - DNA, 127 bp, synthetic construct; identical to SEQ ID NO: 103
  source 1..127: mol_type = other DNA

SEQ ID NO: 520 - DNA, 22 bp, synthetic construct
  source 1..22: mol_type = other DNA
  modified_base 22: mod_base = OTHER; note = Fluorescein modified nucleotide
gggggcgactcagatcccagcc

SEQ ID NO: 521 - DNA, 47 bp, synthetic construct
  source 1..47: mol_type = other DNA
ggtaccgagctcggatccactagtaacggccgccagtgtgctggaat